(19) United States
(12) Patent Application Publication
(10) Pub. No.: US 2023/0119539 A1
(43) Pub. Date: Apr. 20, 2023

(54) TRACELESS LINKERS AND PROTEIN-CONJUGATES THEREOF

(71) Applicant: Regeneron Pharmaceuticals, Inc., Tarrytown, NY (US)

(72) Inventor: Amy HAN, Hockessin, DE (US)

(21) Appl. No.: 17/612,204

(22) PCT Filed: Jan. 8, 2020
(86) PCT No.: PCT/US2020/012798

§ 371 Date: Nov. 17, 2021

Related U.S. Application Data
(63) Continuation-in-part of application No. 16/243,020, filed on Jan. 8, 2019.
Application No. 16/243,020 is a continuation-in-part of application No. PCT/US2019/012786, filed on Jan. 8, 2019.
(60) Provisional application No. 62/872,229, filed on Jul. 9, 2019.
(60) Provisional application No. 62/937,721, filed on Nov. 19, 2019.

(51) Int. Cl. A61K 47/68; C07J 71/00; A61K 47/54; A61K 31/58; A61K 31/685; A61K 31/7048; C07K 16/28; C07K 16/32

(52) U.S. Cl. CPC A61K 47/6803; A61K 47/6889; C07J 71/0031; A61K 47/542; A61K 47/545; A61K 47/549; A61K 47/6849; A61K 47/6855; A61K 31/58; A61K 31/685; A61K 31/7048; C07K 16/2866; C07K 16/2896; C07K 16/32; C07K 2317/41

(57) ABSTRACT

Provided herein are compounds, compounds including traceless linkers, protein conjugates thereof, and compositions thereof. Also provided herein are methods for the treatment of diseases, disorders, and conditions, and/or the management of the symptoms thereof, associated with inflammatory diseases and autoimmune disorders further associated with the glucocorticoid receptor, glucocorticoid binding, and/or glucocorticoid receptor signalling, including administration of the compounds or payloads via traceless linker-payloads, and protein conjugates thereof.

### CROSS-REFERENCE TO RELATED APPLICATIONS

This application claims the benefit of United States Provisional Application No. 62/872,229, filed Jul. 9, 2019; U.S. Provisional Application No. 62/937,721, filed Nov. 19, 2019; PCT Patent Application No. PCT/US2019/012786, titled Steroids and Antibody Conjugates Thereof, which was filed Jan. 8, 2019; and U.S. patent application Ser. No. 16/243,020, titled Steroids and Antibody Conjugates Thereof, which was also filed Jan. 8, 2019. The contents of each priority patent application are incorporated herein by reference in their entireties for all purposes.

### FIELD

Provided herein are novel traceless linkers, and protein conjugates thereof, and methods for treating a variety of diseases, disorders, and conditions including administering compounds or payloads via traceless linker-payloads, and protein conjugates thereof.

### BACKGROUND

Antibody-drug conjugates (ADCs) are antibodies that are covalently linked to biologically active small molecule drugs, often referred to as payloads, thus combining the targeting specificity of antibodies with the mode-of-action and potency of small molecule drugs. The therapeutic utility of ADC(s) has been validated in cancer treatment and is a major ongoing focus of study. ADCETRIS® (bentruximab vedotin) and KADCYLA® (ado-trastuzumab emtansine) are ADCs approved for the treatment of certain cancer types, and several other ADCs are currently in clinical development.

Glucocorticoids (GCs) are small molecule steroids that bind to glucocorticoid receptors (GRs) and are utilized in anti-inflammatory and immunosuppressive therapies. However, due to the ubiquitous expression of glucocorticoid receptors in many cell types, glucocorticoid treatments are compromised by toxicities to most organ systems. Thus, there is need for both novel glucocorticoids as well as novel therapies that minimize the side effects arising from glucocorticoid administration, particularly those arising from activating glucocorticoid receptors in non-target cells. The instant disclosure provides solutions to the aforementioned needs as well as other unmet needs in the field to which the instant disclosure pertains. Included in the instant disclosure are antibody-drug conjugates comprising glucocorticoid payloads.

Liver X Receptor (LXR) includes LXRα and LXRβ which are ligand-dependent transcription factors that control the expression of genes involved in cholesterol, lipid and glucose homeostasis, inflammation, and innate immunity. LXRα is highly expressed in liver, intestine, adipose tissue, and differentiated macrophages; and LXRβ is ubiquitously expressed. LXRs have various biological functions including (i) stimulating the expression of cholesterol transporters, for example, ABCA1 and ABCG1, both of which mediate cellular cholesterol efflux; and (ii) negatively regulating macrophage inflammatory gene expression via repression of NF-kB activation. LXRs have also been implicated in atherosclerosis, proliferative disorders, neurodegenerative disorders, and inflammation. The development of ADCs comprising LXR modulators would allow for target-specific modulation of LXR, thereby avoiding side-effects caused by off-target modulation of LXR. Furthermore, such ADCs would provide improved modulation of biological targets, improved bioavailability, and improved therapeutic window. Therefore, there is a continuing need for effective treatments of, for example, metabolic diseases using small molecule ADCs of LXR modulators.

Linkers covalently link the payload portion, e.g., small molecule therapeutic agent of an ADC to its antibody. A significant challenge in linker design is in finding moieities that keep the payload stably attached to the antibody during storage, formulation, administration, and plasma circulation in the patient, yet allow efficient release upon the antibody binding its target, that allow facile conjugation to the payload under synthesis conditions, and that allow release of the intended payload without alteration in structure. There is a continuing need for linkers that possess these and other attributes.

### SUMMARY

Provided herein are novel traceless linkers, and protein conjugates thereof, and methods for treating a variety of diseases, disorders, and conditions including administering compounds or payloads via traceless linker-payloads, and protein conjugates thereof. Included herein are linkers that bond with a hydroxyl group of a payload and allow release of the payload under appropriate conditions with its hydroxyl group intact.

Provided herein are compounds, compositions, and methods useful for treating, for example, inflammatory diseases and autoimmune disorders, or managing symptoms of any diseases, disorders, or conditions associated with the glucocorticoid receptor, glucocorticoid binding, and/or glucocorticoid receptor signalling; and/or dyslipidemia, a metabolic disease, inflammation, or a neurodegenerative disease, in a subject.

In one embodiment, provided are compounds having the structure of Formula I:

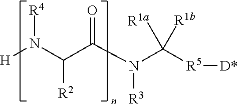

or a pharmaceutically acceptable salt thereof, wherein R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 3-, 4-, 5-, 6-, 7-, or 8-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 3-, 4-, 5-, 6-, 7-, or 8 membered heterocyclyl; R⁴ is hydrogen or alkyl; R⁵ is oxygen, NR⁶, or sulfur; R⁶ is hydrogen or alkyl; D* is acyl, or a residue of a biologically active compound comprising hydroxyl, amino, or thiol; and n is zero, one, two, three, four, five, or six.

In one embodiment, provided are compounds having the structure of Formula I:

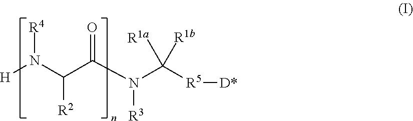

or a pharmaceutically acceptable salt thereof, wherein R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; R⁴ is hydrogen or alkyl; R⁵ is oxygen, NR⁶, or sulfur; R⁶ is hydrogen or alkyl; D* is acyl, or a residue of a biologically active compound comprising hydroxyl, amino, or thiol; and n is zero, one, two, three, four, or five.

In another embodiment, provided are linker-payload compounds having the structure of Formula II:

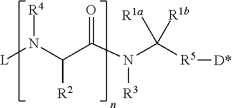

or a pharmaceutically acceptable salt thereof, wherein L is a linker comprising a moiety reactive with an antibody or an antigen binding fragment thereof; R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or hetereoalkylene is further bonded to R³ to form a 3-, 4-, 5-, 6-, 7-, or 8-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 3-, 4-, 5-, 6-, 7-, or 8-membered heterocyclyl; R⁴ is hydrogen or alkyl; R⁵ is oxygen, NR⁶, or sulfur; R⁶ is hydrogen or alkyl; D* is acyl, or a residue of a biologically active compound comprising hydroxyl, amino, or thiol; and n is zero, one, two, three, four, five, or six.

In another embodiment, provided are linker-payloads having the structure of Formula II:

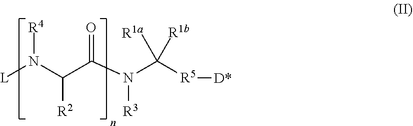

or a pharmaceutically acceptable salt thereof, wherein L is a linker comprising a moiety reactive with an antibody or an antigen binding fragment thereof; R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or hetereoalkylene is further bonded to R³ to form a 4-, 5-, or 6-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; R⁴ is hydrogen or alkyl; R⁵ is oxygen, NR⁶, or sulfur; R⁶ is hydrogen or alkyl; D* is acyl, or a residue of a biologically active compound comprising hydroxyl, amino, or thiol; and n is zero, one, two, three, four, five, or six.

In another embodiment, provided are compounds having the structure of Formula III:

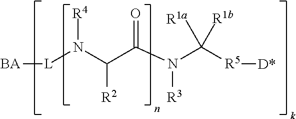

wherein L is a linker; BA is a binding agent; R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 3-, 4-, 5-, 6-, 7-, or 8-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 3-, 4-, 5-, 6-, 7-, or 8-membered heterocyclyl; R⁴ is hydrogen or alkyl; R⁵ is oxygen, NR⁶, or sulfur; R⁶ is hydrogen or alkyl; D* is a residue of a biologically active compound comprising hydroxyl, amino, or thiol; n is zero, one, two, three, four, five, or six; and k is an integer from one to thirty.

In another embodiment, provided are compounds having the structure of Formula III:

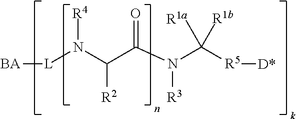

wherein L is a linker; BA is a binding agent; R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 3-, 4-, 5-, 6-, 7-, or 8-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 3-, 4-, 5-, 6-, 7-, or 8-membered heterocyclyl; R⁴ is hydrogen or alkyl; R⁵ is oxygen, NR⁶, or sulfur; R⁶ is hydrogen or alkyl; D* is a residue of an anti-inflammatory biologically active compound comprising hydroxyl, amino, or thiol; n is zero, one, two, three, four, five, or six; and k is an integer from one to thirty.

In another embodiment, provided are compounds having the structure of Formula III:

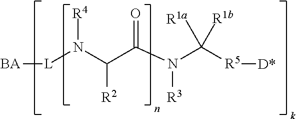

wherein L is a linker comprising PAB or PABC; BA is a binding agent; R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 3-, 4-, 5-, 6-, 7-, or 8-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 3-, 4-, 5-, 6-, 7-, or 8-membered heterocyclyl; R⁴ is hydrogen or alkyl; R⁵ is oxygen, NR⁶, or sulfur; R⁶ is hydrogen or alkyl; D* is a residue of a biologically active compound comprising hydroxyl, amino, or thiol; n is zero, one, two, three, four, five, or six; and k is an integer from one to thirty.

In another embodiment, provided are compounds having the structure of Formula III:

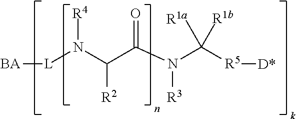

wherein L is a linker; BA is a binding agent; R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 3-, 4-, 5-, 6-, 7-, or 8-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 3-, 4-, 5-, 6-, 7-, or 8-membered heterocyclyl; R⁴ is hydrogen or alkyl; R⁵ is oxygen, NR⁶, or sulfur; R⁶ is hydrogen or alkyl; D* is a residue of a biologically active compound comprising hydroxyl, amino, or thiol; n is zero, one, two, three, four, five, or six; wherein conjugation of L to BA is selected from the group consisting of a click chemistry residue, an amide residue, and a residue comprising two cysteine residues of a single BA that are chemically bonded to L; and k is an integer from one to thirty.

In another embodiment, provided are compounds having the structure of Formula III:

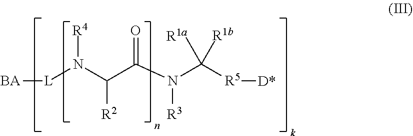

wherein L is a linker; BA is a binding agent; R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5, or 6-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; R⁴ is hydrogen or alkyl; R⁵ is oxygen, NR⁶, or sulfur; R⁶ is hydrogen or alkyl; D* is a residue of a biologically active compound comprising hydroxyl, amino, or thiol; n is zero, one, two, three, four, five, or six; and k is an integer from one to thirty.

In another embodiment, provided are methods for treating a disease, disorder, or condition associated with glucocorticoid receptor signaling in a subject comprising administering to the subject an effective amount of a compound, linker-payload, antibody-drug conjugate, pharmaceutical composition, and/or combinations thereof, as described herein.

In another embodiment, provided are methods for treating dyslipidemia, a metabolic disease, inflammation, or a neurodegenerative disease in a subject comprising administering to the subject an effective amount of a compound, linker-payload, antibody-drug conjugate, pharmaceutical composition, and/or combinations thereof, as described herein.

In another embodiment, provided are methods for treating dyslipidemia in a subject comprising administering to the subject an effective amount of a compound, linker-payload, antibody-drug conjugate, pharmaceutical composition, and/or combinations thereof, as described herein.

In another embodiment, provided are methods for treating a metabolic disease in a subject comprising administering to the subject an effective amount of a compound, linker-payload, antibody-drug conjugate, pharmaceutical composition, and/or combinations thereof, as described herein.

In another embodiment, provided are methods for treating inflammation in a subject comprising administering to the subject an effective amount of a compound, linker-payload, antibody-drug conjugate, pharmaceutical composition, and/or combinations thereof, as described herein.

In another embodiment, provided are methods for treating a neurodegenerative disease in a subject comprising administering to the subject an effective amount of a compound, linker-payload, antibody-drug conjugate, pharmaceutical composition, and/or combinations thereof, as described herein.

### BRIEF DESCRIPTIONS OF THE DRAWING

FIGS. 1-9 show synthetic chemistry schemes for payloads, prodrugs, traceless linkers, traceless linker-payloads, and protein conjugates thereof.

### DESCRIPTION OF EXEMPLARY EMBODIMENTS

### Definitions

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term provided herein, these Definitions prevail unless stated otherwise.

As used herein, “alkyl” refers to a monovalent and saturated hydrocarbon radical moiety. Alkyl is optionally substituted and can be linear, branched, or cyclic, (i.e., cycloalkyl). Alkyl includes, but is not limited to, those radicals having 1-20 carbon atoms, i.e., C₁₋₂₀ alkyl; 1-12 carbon atoms, i.e., C₁₋₁₂ alkyl; 1-8 carbon atoms, i.e., C₁₋₈ alkyl; 1-6 carbon atoms, i.e., C₁₋₆ alkyl; and 1-3 carbon atoms, i.e., C₁₋₃ alkyl. Examples of alkyl moieties include, but are not limited to, methyl, ethyl, n-propyl, i-propyl, n-butyl, s-butyl, t-butyl, i-butyl, a pentyl moiety, a hexyl moiety, cyclopropyl, cyclobutyl, cyclopentyl, and cyclohexyl. A pentyl moiety includes, but is not limited to, n-pentyl and i-pentyl. A hexyl moiety includes, but is not limited to, n-hexyl.

As used herein, “alkylene” refers to a divalent alkyl group. Unless specified otherwise, alkylene includes, but is not limited to, 1-20 carbon atoms. The alkylene group is optionally substituted as described herein for alkyl. In some embodiments, alkylene is unsubstituted. Examples of alkylene moieties include —CH₂—, —CH₂CH₂—, —CH₂CH₂CH₂—, —CH₂CH₂CH₂CH₂—, and the like.

As used herein, “heteroalkylene” refers to a divalent alkyl group wherein one or more carbon atoms is replaced with a heteroatom. Unless specified otherwise, heteroalkylene includes, but is not limited to, 1-20 total atoms (i.e., carbons and heteroatoms). The heteroalkylene group is optionally substituted as described herein for alkyl. In some embodiments, heteroalkylene is unsubstituted. In some embodiments, heteroatoms contemplated within heteroalkylene moieties include oxygen, nitrogen, sulfur (i.e., including sulfoxide, sulphite, sulfate, and sulfone), silicon, and phosphorous (i.e., including phosphite and phosphate), and/or combinations thereof. Nonlimiting exemplary embodiments of heteroalkylene moieties include —CH₂O—, —CH₂OCH₂—, —CH₂OCH₂CH₂—, —CH₂CH₂CH₂OCH₂—, and the like; —CH₂NR—, —CH₂NRCH₂—, —CH₂NRCH₂CH₂—, —CH₂CH₂CH₂NRCH₂—, and the like; and —CH₂S—, —CH₂SCH₂—, —CH₂SCH₂CH₂—, —CH₂CH₂CH₂SCH₂—, and the like.

Designation of an amino acid or amino acid residue without specifying its stereochemistry is intended to encompass the L-form of the amino acid, the D-form of the amino acid, or a racemic mixture thereof.

As used herein, “haloalkyl” refers to alkyl, as defined above, wherein the alkyl includes at least one substituent selected from a halogen, for example, fluorine (F), chlorine (Cl), bromine (Br), or iodine (I). Examples of haloalkyl include, but are not limited to, —CF₃, —CH₂CF₃, —CCl₂F, —CHF₂, and —CCl₃.

As used herein, “alkenyl” refers to a monovalent hydrocarbon radical moiety containing at least two carbon atoms and one or more non-aromatic carbon-carbon double bonds. Alkenyl is optionally substituted and can be linear, branched, or cyclic. Alkenyl includes, but is not limited to, those radicals having 2-20 carbon atoms, i.e., C₂₋₂₀ alkenyl; 2-12 carbon atoms, i.e., C₂₋₁₂ alkenyl; 2-8 carbon atoms, i.e., C₂₋₄ alkenyl; 2-6 carbon atoms, i.e., C₂₋₆ alkenyl; and 2-4 carbon atoms, i.e., C₂₋₄ alkenyl. Examples of alkenyl moieties include, but are not limited to, vinyl, propenyl, butenyl, and cyclohexenyl.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

As used herein, “alkoxy” refers to a monovalent and saturated hydrocarbon radical moiety wherein the hydrocarbon includes a single bond to an oxygen atom and wherein the radical is localized on the oxygen atom, e.g., CH₃CH₂—O. for ethoxy. Alkoxy substituents bond to the compound which they substitute through this oxygen atom of the alkoxy substituent. Alkoxy is optionally substituted and can be linear, branched, or cyclic, i.e., cycloalkoxy. Alkoxy includes, but is not limited to, those having 1-20 carbon atoms, i.e., C₁₋₂₀ alkoxy; 1-12 carbon atoms, i.e., C₁₋₁₂ alkoxy; 1-8 carbon atoms, i.e., C₁₋₈ alkoxy; 1-6 carbon atoms, i.e., C₁₋₈ alkoxy; and 1-3 carbon atoms, i.e., C₁₋₃ alkoxy. Examples of alkoxy moieties include, but are not limited to, methoxy, ethoxy, n-propoxy, i-propoxy, n-butoxy, s-butoxy, 1-butoxy, i-butoxy, a pentoxy moiety, a hexoxy moiety, cyclopropoxy, cyclobutoxy, cyclopentoxy, and cyclohexoxy.

As used herein, “haloalkoxy” refers to alkoxy, as defined above, wherein the alkoxy includes at least one substituent selected from a halogen, e.g., F, Cl, Br, or I.

As used herein, “aryl” refers to a monovalent moiety that is a radical of an aromatic compound wherein the ring atoms are carbon atoms. Aryl is optionally substituted and can be monocyclic or polycyclic, e.g., bicyclic or tricyclic. Examples of aryl moieties include, but are not limited to, those having 6 to 20 ring carbon atoms, i.e., C₆₋₂₀ aryl; 6 to 15 ring carbon atoms, i.e., C₆₋₁₅ aryl, and 6 to 10 ring carbon atoms, i.e., C₆₋₁₀ aryl. Examples of aryl moieties include, but are limited to, phenyl, naphthyl, fluorenyl, azulenyl, anthryl, phenanthryl, and pyrenyl.

As used herein, “arylalkyl” refers to a monovalent moiety that is a radical of an alkyl compound, wherein the alkyl compound is substituted with an aromatic substituent, i.e., the aromatic compound includes a single bond to an alkyl group and wherein the radical is localized on the alkyl group. An arylalkyl group bonds to the illustrated chemical structure via the alkyl group. An arylalkyl can be represented by the structure, e.g.,

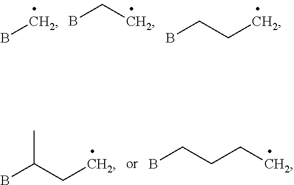

where B is an aromatic moiety, e.g., aryl or phenyl. Arylalkyl is optionally substituted, i.e., the aryl group and/or the alkyl group, can be substituted as disclosed herein. Examples of arylalkyl include, but are not limited to, benzyl.

As used herein, “alkylaryl” refers to a monovalent moiety that is a radical of an aryl compound, wherein the aryl compound is substituted with an alkyl substituent, i.e., the aryl compound includes a single bond to an alkyl group and wherein the radical is localized on the aryl group. An alkylaryl group bonds to the illustrated chemical structure via the aryl group. An alkylaryl can be represented by the structure, e.g.,

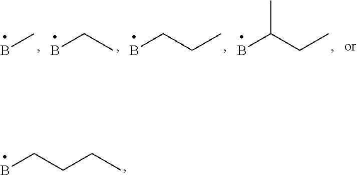

wherein B is an aromatic moiety, e.g., phenyl. Alkylaryl is optionally substituted, i.e., the aryl group and/or the alkyl group, can be substituted as disclosed herein. Examples of alkylaryl include, but are not limited to, toluyl.

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g.,

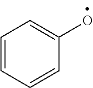

for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C₆₋₁₅ aryloxy, and 6 to 10 ring carbon atoms, i.e., C₆₋₁₀ aryloxy. Examples of aryloxy moieties include, but are not limited to phenoxy, naphthoxy, and anthroxy.

As used herein, “arylene” refers to a divalent moiety of an aromatic compound wherein the ring atoms are only carbon atoms. Arylene is optionally substituted and can be monocyclic or polycyclic, e.g., bicyclic or tricyclic. Examples of arylene moieties include, but are not limited to those having 6 to 20 ring carbon atoms, i.e., C₆₋₂₀ arylene; 6 to 15 ring carbon atoms, i.e., C₆₋₁₅ arylene, and 6 to 10 ring carbon atoms, i.e., C₆₋₁₀ arylene.

As used herein, “heteroalkyl” refers to an alkyl in which one or more carbon atoms are replaced by heteroatoms. As used herein, “heteroalkenyl” refers to an alkenyl in which one or more carbon atoms are replaced by heteroatoms. As used herein, “heteroalkynyl” refers to an alkynyl in which one or more carbon atoms are replaced by heteroatoms. Suitable heteroatoms include, but are not limited to, nitrogen, oxygen, and sulfur atoms. Heteroalkyl, heteroalkenyl, and heteroalkynyl are optionally substituted. Examples of heteroalkyl moieties include, but are not limited to, aminoalkyl, sulfonylalkyl, and sulfinylalkyl. Examples of heteroalkyl moieties also include, but are not limited to, methylamino, methylsulfonyl, and methylsulfinyl.

As used herein, “heteroaryl” refers to a monovalent moiety that is a radical of an aromatic compound wherein the ring atoms contain carbon atoms and at least one oxygen, sulfur, nitrogen, or phosphorus atom. Examples of heteroaryl moieties include, but are not limited to those having 5 to 20 ring atoms; 5 to 15 ring atoms; and 5 to 10 ring atoms. Heteroaryl is optionally substituted.

As used herein, “heteroarylene” refers to a divalent heteroaryl in which one or more ring atoms of the aromatic ring are replaced with an oxygen, sulfur, nitrogen, or phosphorus atom. Heteroarylene is optionally substituted.

As used herein, “heterocycloalkyl” or “heterocyclyl” refers to a cycloalkyl in which one or more carbon atoms are replaced by heteroatoms. Suitable heteroatoms include, but are not limited to, nitrogen, oxygen, and sulfur atoms (i.e., including sulfoxide and sulfone). Heterocycloalkyl or heterocyclyl is optionally substituted. Examples of heterocycloalkyl and heterocyclyl moieties include, but are not limited to, morpholinyl, piperidinyl, tetrahydropyranyl, pyrrolidinyl, aziridnyl, imidazolidinyl, oxazolidinyl, thiazolidinyl, dioxolanyl, dithiolanyl, oxanyl, or thianyl.

As used herein, “Lewis acid” refers to a molecule or ion that accepts an electron lone pair. The Lewis acids used in the methods described herein are those other than protons. Lewis acids include, but are not limited to, non-metal acids, metal acids, hard Lewis acids, and soft Lewis acids. Lewis acids include, but are not limited to, Lewis acids of aluminum, boron, iron, tin, titanium, magnesium, copper, antimony, phosphorus, silver, ytterbium, scandium, nickel, and zinc. Illustrative Lewis acids include, but are not limited to, AlBr₃, AlCl₃, BCl₃, boron trichloride methyl sulfide, BF₃, boron trifluoride methyl etherate, boron trifluoride methyl sulfide, boron trifluoride tetrahydrofuran, dicyclohexylboron trifluoromethanesulfonate, iron (III) bromide, iron (III) chloride, tin (IV) chloride, titanium (IV) chloride, titanium (IV) isopropoxide, Cu(OTf)₂, CuCl₂, CuBr₂, zinc chloride, alkylaluminum halides (RₙAlX₃₋ₙ, wherein R is hydrocarbyl), Zn(OTf)₂, ZnCl₂, Yb(OTf)₃, Sc(OTf)₃, MgBr₂, NiCl₂, Sn(OTf)₂, Ni(OTf)₂, and Mg(OTf)₂.

As used herein, “N-containing heterocycloalkyl,” refers to a cycloalkyl in which one or more carbon atoms are replaced by heteroatoms and wherein at least one replacing heteroatom is a nitrogen atom. Suitable heteroatoms in addition to nitrogen, include, but are not limited to, oxygen and sulfur atoms. N-containing heterocycloalkyl is optionally substituted. Examples of N-containing heterocycloalkyl moieties include, but are not limited to, morpholinyl, piperidinyl, pyrrolidinyl, imidazolidinyl, oxazolidinyl, or thiazolidinyl.

As used herein, “optionally substituted,” when used to describe a radical moiety, for example, optionally substituted alkyl, means that such moiety is optionally bonded to one or more substituents. Examples of such substituents include, but are not limited to, halo, cyano, nitro, amino, hydroxyl, optionally substituted haloalkyl, aminoalkyl, hydroxyalkyl, azido, epoxy, optionally substituted heteroaryl, optionally substituted heterocycloalkyl,

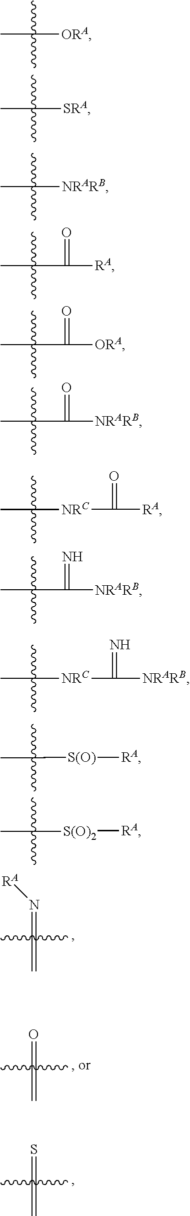

wherein R^{A}, R^{B}, and R^{C} are, independently at each occurrence, a hydrogen atom, alkyl, alkenyl, alkynyl, aryl, alkylaryl, arylalkyl, heteroalkyl, heteroaryl, or heterocycloalkyl, or R^{A} and R^{B} together with the atoms to which they are bonded, form a saturated or unsaturated carbocyclic ring, wherein the ring is optionally substituted, and wherein one or more ring atoms is optionally replaced with a heteroatom. In certain embodiments, when a radical moiety is optionally substituted with an optionally substituted heteroaryl, optionally substituted heterocycloalkyl, or optionally substituted saturated or unsaturated carbocyclic ring, the substituents on the optionally substituted heteroaryl, optionally substituted heterocycloalkyl, or optionally substituted saturated or unsaturated carbocyclic ring, if they are substituted, are not substituted with substituents which are further optionally substituted with additional substituents. In some embodiments, when a group described herein is optionally substituted, the substituent bonded to the group is unsubstituted unless otherwise specified.

As used herein, “binding agent” refers to any molecule, e.g., protein, antibody, or fragment thereof, capable of binding with specificity to a given binding partner, e.g., antigen.

As used herein, “linker” refers to a divalent, trivalent, or multivalent moiety that covalently links, or is capable of covalently linking (e.g., via a reactive group), the binding agent to one or more compounds described herein, for instance, payload compounds and enhancement agents.

As used herein, “amide synthesis conditions” refers to reaction conditions suitable to effect the formation of an amide, e.g., by the reaction of a carboxylic acid, activated carboxylic acid, or acyl halide with an amine. In some examples, amide synthesis conditions refers to reaction conditions suitable to effect the formation of an amide bond between a carboxylic acid and an amine. In some of these examples, the carboxylic acid is first converted to an activated carboxylic acid before the activated carboxylic acid reacts with an amine to form an amide. Suitable conditions to effect the formation of an amide include, but are not limited to, those utilizing reagents to effect the reaction between a carboxylic acid and an amine, including, but not limited to, dicyclohexylcarbodiimide (DCC), diisopropylcarbodiimide (DIC), (benzotriazol-1-yloxy)tris(dimethylamino)phosphonium hexafluorophosphate (BOP), (benzotriazol-1-yloxy)tripyrrolidinophosphonium hexafluorophosphate (PyBOP), (7-azabenzotriazol-1-yloxy)tripyrrolidinophosphonium hexafluorophosphate (PyAOP), bromotripyrrolidinophosphonium hexafluorophosphate (PyBrOP), O-(benzotriazol-1-yl)-N,N,N′,N′-tetramethyluronium hexafluorophosphate (HBTU), O-(benzotriazol-1-yl)-N,N,N′,N′-tetramethyluronium tetrafluoroborate (TBTU), 1-[Bis(dimethylamino)methyl ene]-1H-1,2,3-triazolo[4,5-b]pyridinium 3-oxide hexafluorophosphate (HATU), N-ethoxycarbonyl-2-ethoxy-1,2-dihydroquinoline (EEDQ), N-ethyl-N′-(3-dimethylaminopropyl)carbodiimide (EDC), 2-chloro-1,3-dimethylimidazolidinium hexafluorophosphate (CIP), 2-chloro-4,6-dimethoxy-1,3,5-triazine (CDMT), and carbonyldiimidazole (CDI). In some examples, a carboxylic acid is first converted to an activated carboxylic ester before treating the activated carboxylic ester with an amine to form an amide bond. In certain embodiments, the carboxylic acid is treated with a reagent. The reagent activates the carboxylic acid by deprotonating the carboxylic acid and then forming a product complex with the deprotonated carboxylic acid as a result of nucleophilic attack by the deprotonated carboxylic acid onto the protonated reagent. The activated carboxylic esters for certain carboxylic acids are subsequently more susceptible to nucleophilic attack by an amine than the carboxylic acid is before it is activated. This results in amide bond formation. As such, the carboxylic acid is described as activated. Exemplary reagents include DCC and DIC.

As used herein, “regioisomer,” “regioisomers,” or “mixture of regioisomers” refers to the product(s) of 1,3-cycloadditions or strain-promoted alkyne-azide cycloadditions (SPAACs)—otherwise known as click reactions—that derive from suitable azides (e.g., —N₃, or -PEG-N₃ derivitized antibodies) treated with suitable alkynes. In certain embodiments, for example, regioisomers and mixtures of regioisomers are characterized by the click reaction products shown below:

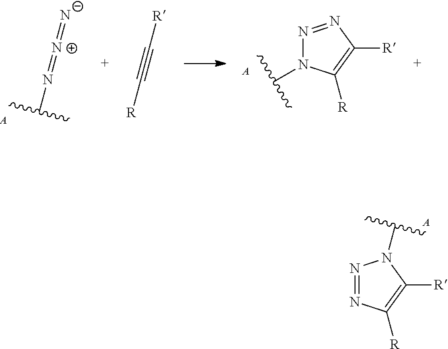

In certain embodiments, more than one suitable azide and more than one suitable alkyne can be utilized within a synthetic scheme en route to a product, where each pair of azide-alkyne can participate in one or more independent click reactions to generate a mixture of regioisomeric click reaction products. For example, a person of skill will recognize that a first suitable azide may independently react with a first suitable alkyne, and a second suitable azide may independently react with a second suitable alkyne, en route to a product, resulting in the generation of four possible click reaction regioisomers or a mixture of the four possible click reaction regioisomers.

As used herein, the term “residue” refers to the chemical moiety within a compound that remains after a chemical reaction. For example, the term “amino acid residue” or “N-alkyl amino acid residue” refers to the product of an amide coupling or peptide coupling of an amino acid or a N-alkyl amino acid to a suitable coupling partner; wherein, for example, a water molecule is expelled after the amide or peptide coupling of the amino acid or the N-alkylamino acid, resulting in the product having the amino acid residue or N-alkyl amino acid residue incorporated therein.

As used herein, “therapeutically effective amount” refers to an amount (e.g., of a compound) that is sufficient to provide a therapeutic benefit to a patient in the treatment or management of a disease or disorder, or to delay or minimize one or more symptoms associated with the disease or disorder.

As used herein, “constitutional isomers” refers to compounds that have the same molecular formula, but different chemical structures resulting from the way the atoms are arranged. Exemplary constitutional isomers include n-propyl and isopropyl; n-butyl, sec-butyl, and tert-butyl; and n-pentyl, isopentyl, and neopentyl, and the like.

Certain groups, moieties, substituents, and atoms are depicted with a wiggly line that intersects a bond or bonds to indicate the atom through which the groups, moieties, substituents, atoms are bonded. For example, a phenyl group that is substituted with a propyl group depicted as:

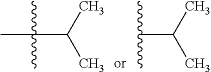

has the following structure:

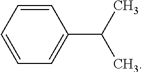

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains. For example, the group,

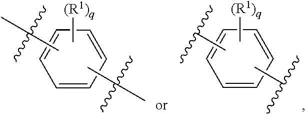

wherein subscript q is an integer from 0 to 4 and in which the positions of substituent R¹ are described generically, i.e., not directly attached to any vertex of the bond line structure, i.e., specific ring carbon atom, includes the following, non-limiting examples of groups in which the substituent R¹ is bonded to a specific ring carbon atom:

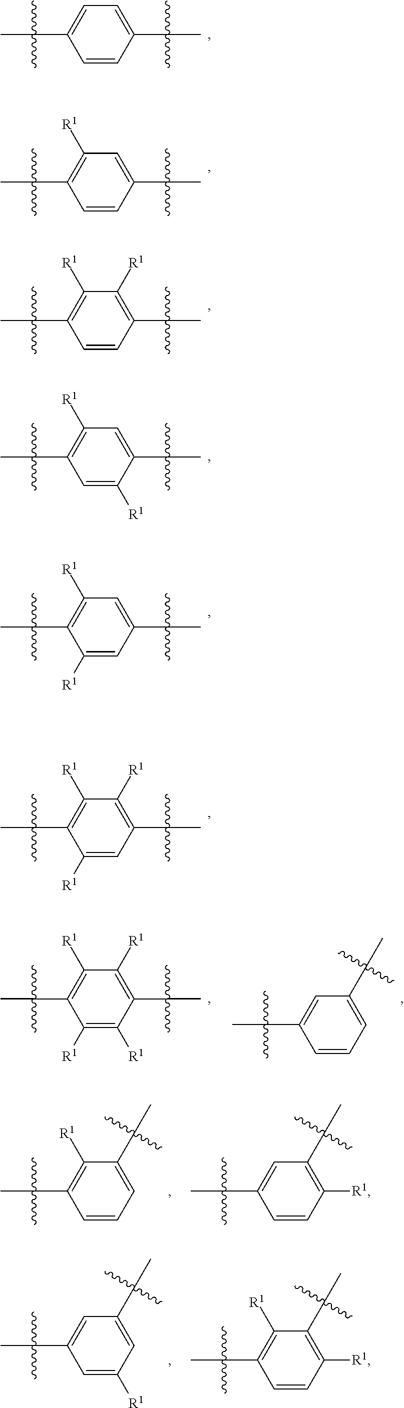

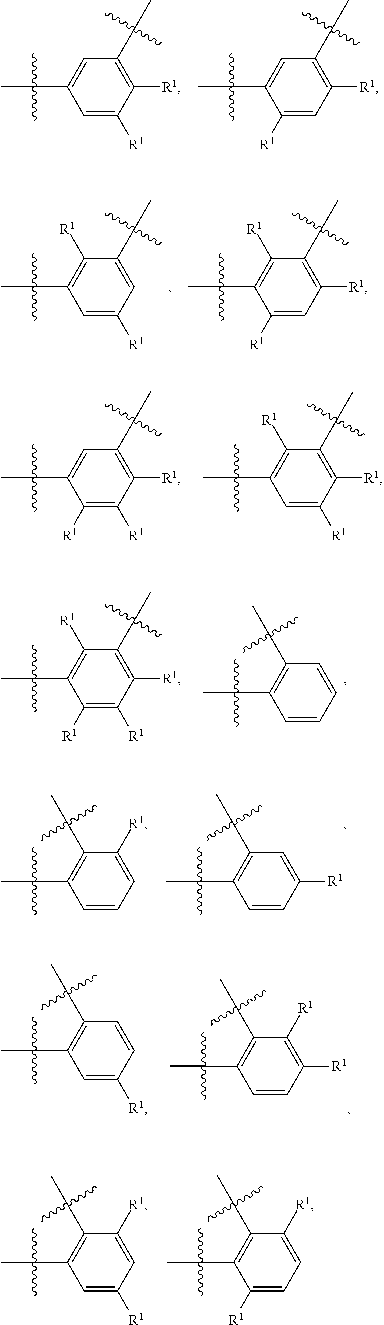

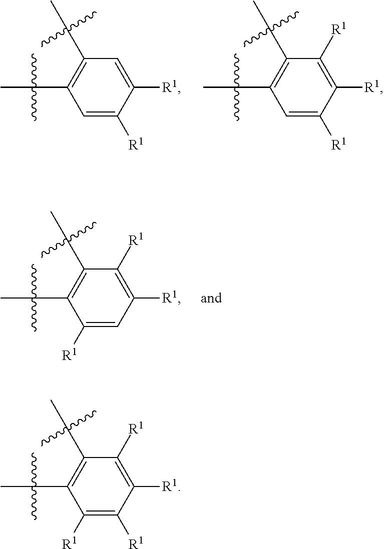

As used herein, the phrase “reactive linker,” or the abbreviation “RL” refers to a monovalent group that includes a reactive group (“RG”) and spacer group (“SP”), depicted for example as

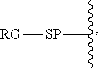

wherein RG is the reactive group and SP is the spacer group. As described herein, a reactive linker may include more than one reactive group and more than one spacer group. The spacer group is any divalent moiety that bridges the reactive group to another group, such as a payload (e.g., a biologically active compound). The reactive linkers (RLs), together with the payloads to which they are bonded, provide intermediates (“linker-payloads” or LPs) useful as synthetic precursors for the preparation of the antibody conjugates described herein. The reactive linker includes a reactive group, which is a functional group or moiety that is capable of reacting with a reactive portion of another group, for instance, an antibody, modified antibody, or antigen binding fragment thereof, or an enhancement group. The moiety resulting from the reaction of the reactive group with the antibody, modified antibody, or antigen binding fragment thereof, together with the linking group, include the “binding agent linker” (“BL”) portion of the conjugate, described herein. In certain embodiments, the “reactive group” is a functional group or moiety (e.g., maleimide or N-hydroxysuccinimide (NETS) ester) that reacts with a cysteine or lysine residue of an antibody or antigen-binding fragment thereof. In certain embodiments, the “reactive group” is a functional group or moiety that is capable of undergoing a click chemistry reaction (see, e.g., click chemistry, Huisgen Proc. Chem. Soc. 1961, Wang et al. J. Am. Chem. Soc. 2003, and Agard et al. J. Am. Chem. Soc. 2004). In some embodiments of said click chemistry reaction, the reactive group is an alkyne that is capable of undergoing a 1,3-cycloaddition reaction with an azide. Such suitable reactive groups include, but are not limited to, strained alkynes, e.g., those suitable for strain-promoted alkyne-azide cycloadditions (SPAAC), cycloalkynes, e.g., cyclooctynes, benzannulated alkynes, and alkynes capable of undergoing 1,3-cycloaddition reactions with alkynes in the absence of copper catalysts. Suitable alkynes also include, but are not limited to,

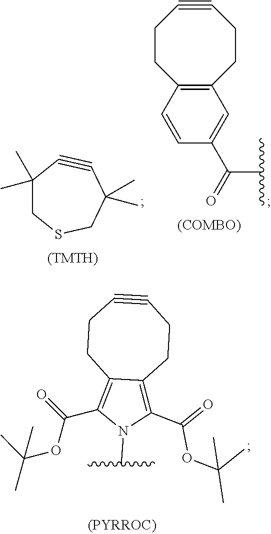

cyclooctyne

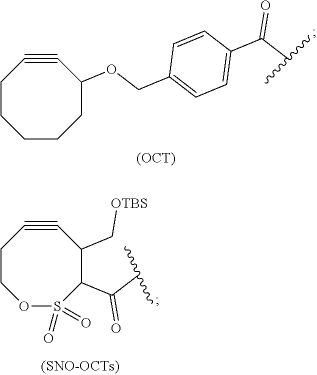 [SMILES: *C(=O)c1ccc(COC2C#CCCCCC2)cc1 ; "(OCT)" ; *C(=O)C1C(CO[Si](C)(C)C(C)(C)C)C#CCCOS1(=O)=O]

azacyclooctyne

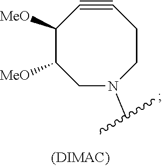 [SMILES: *N1CCC#C[C@H](OC)[C@@H](OC)C1]

diabenzoazacyclooctyne or

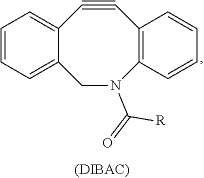

dibenzocyclooctyne or

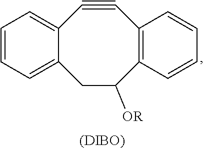

biarylazacyclooctynone of

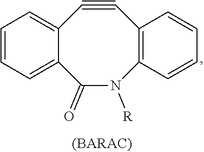

monofluorinated cyclooctyne

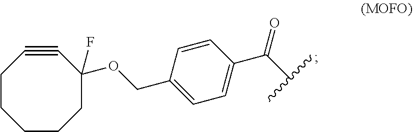 [SMILES: *C(=O)c1ccc(COC2(F)C#CCCCCC2)cc1]

difluorinated cyclooctyne or

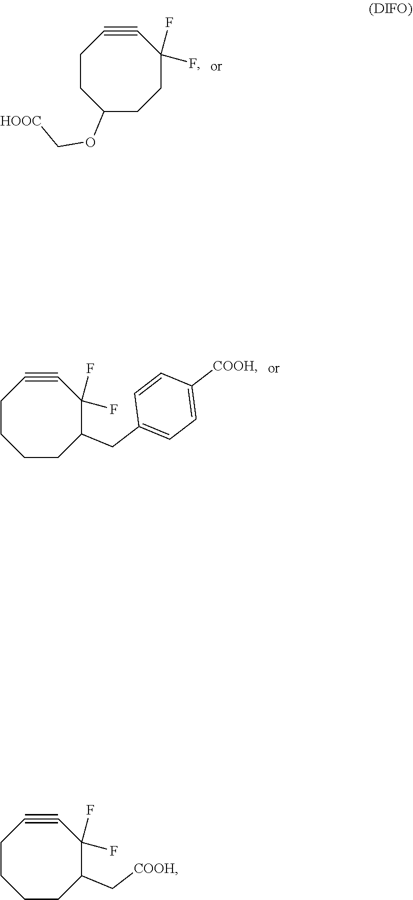 [SMILES: O=C(O)COC1CCC#CC(F)(F)CC1 ; O=C(O)c1ccc(CC2CCCCC#CC2(F)F)cc1 ; O=C(O)CC1CCCCC#CC1(F)F]

substituted, e.g., fluorinated alkynes, aza-cycloalkynes, bicycle[6.1.0]nonyne or

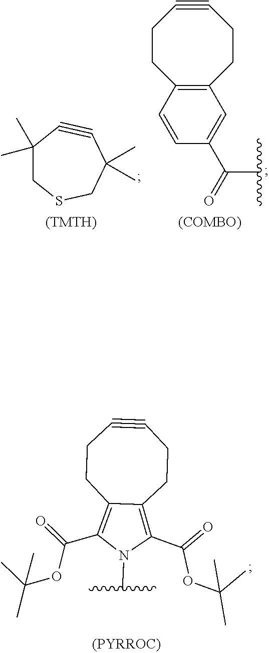

where R is alkyl, alkoxy, or acyl), and derivatives thereof. Particularly useful alkynes include

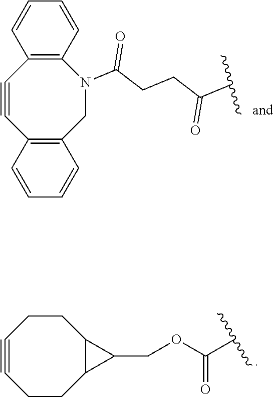 [SMILES: *C(=O)CCC(=O)N1Cc2ccccc2C#Cc2ccccc21 ; *C(=O)OCC1C2CCC#CCCC21]

Linker-payloads including such reactive groups are useful for conjugating antibodies that have been functionalized with azido groups. Such functionalized antibodies include antibodies functionalized with azido-polyethylene glycol groups. In certain embodiments, such a functionalized antibody is derived by treating an antibody having at least one glutamine residue, e.g., heavy chain Gln295, with a compound bearing an amino agroup and an azide group, in the presence of the enzyme transglutaminase.

In some examples, the reactive group is an alkyne, e.g.,

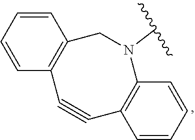 [SMILES: *N1Cc2ccccc2C#Cc2ccccc21]

which can react via click chemistry with an azide, e.g.,

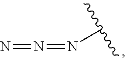

to form a click chemistry product, e.g.,

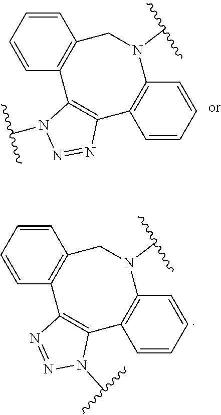 [SMILES: *N1Cc2ccccc2-c2c(nnn2*)-c2ccccc21 ; *N1Cc2ccccc2-c2nnn(*)c2-c2ccccc21]

In some examples, the group reacts with an azide on a modified antibody or antigen binding fragment thereof. In some examples, the reactive group is an alkyne, e.g.,

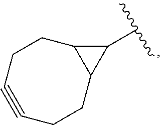 [SMILES: *C1C2CCC#CCCC12]

which can react via click chemistry with an azide, e.g.,

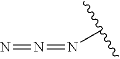

to form a click chemistry product, e.g.,

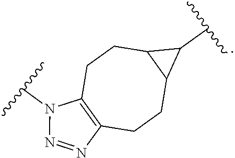 [SMILES: *C1C2CCc3nnn(*)c3CCC12]

In some examples, the reactive group is an alkyne, e.g.,

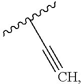 [SMILES: *C#C]

which can react via click chemistry with an azide, e.g.,

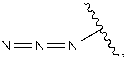

to form a click chemistry product, e.g.,

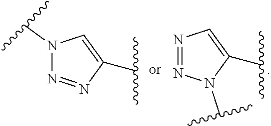

In some examples, the reactive group is a functional group, e.g.,

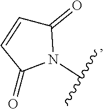 [SMILES: *N1C(=O)C=CC1=O]

which reacts with a cysteine residue on an antibody or antigen-binding fragment thereof, to form a carbon-sulfur bond thereto, e.g.,

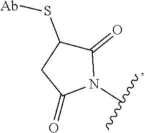

wherein Ab refers to an antibody or antigen-binding fragment thereof and S refers to the S atom on a cysteine residue through which the functional group bonds to the Ab. In some examples, the reactive group is a functional group, e.g.,

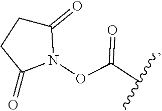 [SMILES: *C(=O)ON1C(=O)CCC1=O]

which reacts with a lysine residue on an antibody or antigen-binding fragment thereof, to form an amide bond thereto, e.g.,

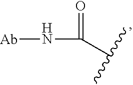

wherein Ab refers to an antibody or antigen-binding fragment thereof and NH refers to the NH atom on a lysine side chain residue through which the functional group bonds to the Ab.

As used herein, the phrase “biodegradable moiety” refers to a moiety that degrades in vivo to non-toxic, biocompatible components which can be cleared from the body by ordinary biological processes. In some embodiments, a biodegradable moiety completely or substantially degrades in vivo over the course of about 90 days or less, about 60 days or less, or about 30 days or less, where the extent of degradation is based on percent mass loss of the biodegradable moiety, and wherein complete degradation corresponds to 100% mass loss. Exemplary biodegradable moieties include, without limitation, aliphatic polyesters such as poly(ε-caprolactone) (PCL), poly(3-hydroxybutyrate) (PHB), poly(glycolic acid) (PGA), poly(lactic acid) (PLA) and its copolymers with glycolic acid (i.e., poly(D,L-lactide-coglycolide) (PLGA) (Vert M, Schwach G, Engel R and Coudane J (1998) J Control Release 53(1-3):85-92; Jain R A (2000) Biomaterials 21(23):2475-2490; Uhrich K E, Cannizzaro S M, Langer R S and Shakesheff K M (1999) Chemical Reviews 99(11): 3181-3198; and Park T G (1995) Biomaterials 16(15):1123-1130, each of which are incorporated herein by reference in their entirety).

As used herein, the phrase “binding agent linker,” or “BL” refers to any divalent, trivalent, or multi-valent group or moiety that links, connects, or bonds a binding agent (e.g., an antibody or an antigen-binding fragment thereof) with a payload compound set forth herein (e.g., tubulysins) and, optionally, with one or more side chain compounds. Generally, suitable binding agent linkers for the antibody conjugates described herein are those that are sufficiently stable to exploit the circulating half-life of the antibody conjugates and, at the same time, capable of releasing its payload after antigen-mediated internalization of the conjugate. Linkers can be cleavable or non-cleavable. Cleavable linkers are linkers that are cleaved by intracellular metabolism following internalization, e.g., cleavage via hydrolysis, reduction, or enzymatic reaction. Non-cleavable linkers are linkers that release an attached payload via lysosomal degradation of the antibody following internalization. Suitable linkers include, but are not limited to, acid-labile linkers, hydrolytically-labile linkers, enzymatically cleavable linkers, reduction labile linkers, self-immolative linkers, and non-cleavable linkers. Suitable linkers also include, but are not limited to, those that are or comprise peptides, glucuronides, succinimide-thioethers, polyethylene glycol (PEG) units, hydrazones, mal-caproyl units, dipeptide units, valine-citruline units, and para-aminobenzyloxycarbonyl (PABC), para-aminobenzyl (PAB) units. In some embodiments, the binding agent linker (BL) includes a moiety that is formed by the reaction of the reactive group (RG) of a reactive linker (RL) and reactive portion of the binding agent, e.g., antibody, modified antibody, or antigen binding fragment thereof.

In some examples, the BL includes the following moiety:

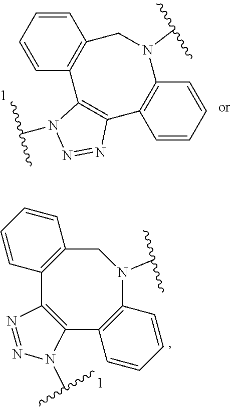 [SMILES: *N1Cc2ccccc2-c2c(nnn2*)-c2ccccc21 ; *N1Cc2ccccc2-c2nnn(*)c2-c2ccccc21]

wherein

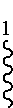

is the bond to the binding agent. In some examples, the BL includes the following moiety:

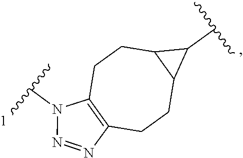 [SMILES: *C1C2CCc3nnn(*)c3CCC12]

wherein

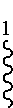

is the bond to the binding agent. In some examples, the BL includes the following moiety:

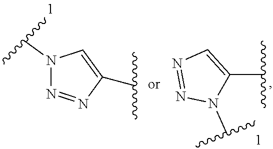 [SMILES: *c1cn(*)nn1 ; *c1cnnn1*]

wherein

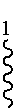

is the bond to the binding agent. In some examples, the BL includes the following moiety:

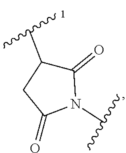 [SMILES: *C1CC(=O)N(*)C1=O]

wherein

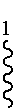

is the bond to the cysteine of the antibody or antigen-binding fragment thereof. In some examples, the BL includes the following moiety:

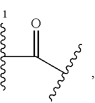 [SMILES: *C(*)=O]

wherein

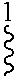

is the bond to the lysine of the antibody or antigen-binding fragment thereof.

As used herein, “amino acid side chain” refers to the additional chemical moiety on the same carbon that bears a primary or secondary amine and a carboxylic acid of an amino acid. As would be appreciated by a person of skill in the art, there are twenty-one “standard” amino acids. Exemplary “standard” amino acids include, without limitation, alanine, serine, proline, arginine, and aspartic acid. Other amino acids include, cysteine, selenocysteine, and glycine (e.g., wherein the additional chemical moiety on the same carbon that bears the primary amine and carboxylic acid of glycine is hydrogen). Exemplary amino acid side chains include, without limitation, methyl (i.e., alanine), sec-buytl (i.e., isoleucine), iso-butyl (i.e., leucine), —CH₂CH₂SCH₃ (i.e., methionine), —CH₂Ph (i.e., phenylalanine),

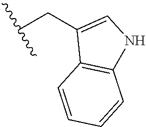 [SMILES: *Cc1c[nH]c2ccccc12]

(i.e., tryptophan),

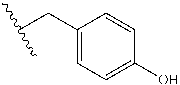 [SMILES: *Cc1ccc(O)cc1]

(i.e., tyrosine), iso-propyl (i.e., valine), hydroxymethyl (i.e., serine), —CH(OH)CH₃ (i.e., threonine), —CH₂C(O)NH₂ (i.e., asparagine), —CH₂CH₂C(O)NH₂ (i.e., glutamine), —CH₂SH (i.e., cysteine), —CH₂SeH (i.e., selenocysteine), —CH₂NH₂ (i.e., glycine), propylene or —CH₂CH₂CH₂— (i.e., proline), —CH₂CH₂CH₂NHC(═NH)NH₂ (i.e., arginine),

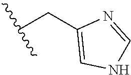 [SMILES: *Cc1c[nH]cn1]

(i.e., histidine), —CH₂CH₂CH₂CH₂NH₂ (i.e., lysine), —CH₂COOH (i.e., aspartic acid), and —CH₂CH₂COOH (i.e., glutamic acid).

As used herein, “biologically active compound” refers to a compound, prodrug, or payload that elicits a biological response when administered to a biological entity. Exemplary biological responses include, without limitation, increase or decrease in DNA or protein synthesis, up-regulation or down-regulation of signalling pathways, and increase or decrease in cell proliferation, and the like.

### Compounds, Prodrugs, or Payloads

Provided herein are compounds or payloads. Without being bound by any particular theory of operation, the compounds include anti-inflammatory biologically active compounds, steroids, steroid derivatives, and/or LXR modulators, and derivatives thereof, for example, prodrugs thereof. The terms or phrases “compounds,” “biologically active compounds,” “prodrugs,” and “payloads” are used interchangeably throughout this disclosure. In certain embodiments, the biologically active compound (D*) or residue thereof includes hydroxyl functionality (e.g., D*-OH or D*-O—R). In certain embodiments herein, for example and convenience, R⁵ represents the hydroxyl, amino, and thiol functional groups within the biologically active compounds described herein, as would be appreciated by a person of skill, or a portion thereof such as —O—, —N(R)—, or —S—. Alternatively stated, a person of skill would recognize that R⁵ may be part of the biologically active compounds described herein (e.g., D*), and may be used as a functional group for conjugation purposes. In one embodiment, the hydroxyl functionality is a primary hydroxyl moiety (e.g., D*-CH₂OH or D*-CH₂O—R; or D*-C(O)CH₂OH or D*-C(O)CH₂O—R). In another embodiment, the hydroxyl functionality is a secondary hydroxyl moiety (e.g., D*-CH(OH)R or D*-CH(O—R)R; or D*-C(O)CH(R)(OH) or D*-C(O)CH(R)(O—R)). In another embodiment, the hydroxyl functionality is a tertiary hydroxyl moiety (e.g., D*-C(R₁)(R₂)(OH) or D*-C(R₁)(R₂)(O—R); or D*-C(O)C(R₁)(R₂)(OH) or D*-C(O)C(R₁)(R₂)(O—R)). Those of skill will recognize that each functional group in the previous sentences can be part of the biologically active compound D* and simultaneously be depicted in the formula for clarity, convenience, and/or emphasis. In another embodiment, the D* including the hydroxyl functionality is an aryl hydroxyl or phenolic hydroxyl (e.g., D*-Ar-OH, D*-Ar—O—R. In one embodiment, the biologically active compound (D*) including hydroxyl functionality (D*-OH) is dexamethasone, and the residue including the hydroxyl functionality is

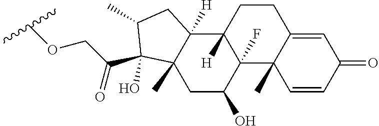

wherein [symbol] indicates attachment to a prodrug moiety (as shown in Formulae Ia and/or Ib), a linker, and/or binding agent, as described herein. In one embodiment, the biologically active compound (D*) including hydroxyl functionality is dexamethasone, and the residue including the hydroxyl functionality is

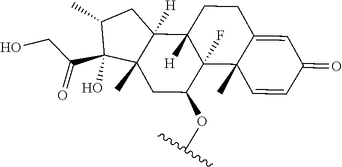

wherein [symbol] indicates attachment to a prodrug moiety (as shown in Formulae Ia and/or Ib), a linker, and/or binding agent, as described herein. In one embodiment, the biologically active compound (D*) including hydroxyl functionality (D*-OH) is budesonide, and the residue including the hydroxyl functionality is

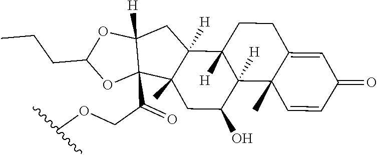

wherein [symbol] indicates attachment to a prodrug moiety (as shown in Formulae Ia and/or Ib), a linker, and/or binding agent, as described herein. In one embodiment, the biologically active compound (D*) including hydroxyl functionality is budesonide, and the residue including the hydroxyl functionality is

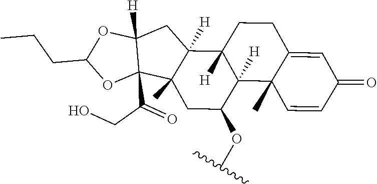

wherein [symbol] indicated attachment to a prodrug moiety (as shown in Formulae Ia and/or Ib), a linker, and/or binding agent, as described herein. In one embodiment, the biologically active compound (D*) including hydroxyl functionality (D*-OH) is 6,11-2F-budesonide, and the residue including the hydroxyl functionality is

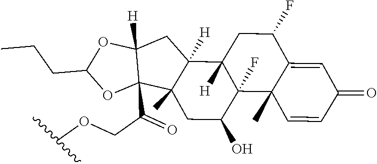

wherein [symbol] indicates attachment to a prodrug moiety (as shown in Formulae Ia and/or Ib), a linker, and/or binding agent, as described herein. In one embodiment, the biologically active compound (D*) including hydroxyl functionality is 6,11-2F-budesonide, and the residue including the hydroxyl functionality is

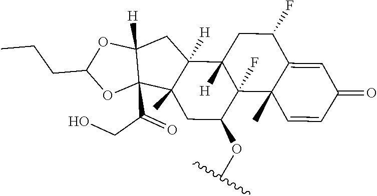

wherein [symbol] attachment to a prodrug moiety (as shown in Formulae Ia and/or Ib), a linker, and/or binding agent, as described herein. In one embodiment, the biologically active compound (D*) including hydroxyl functionality (D*-OH) is an LXR agonist, and the residue including the hydroxyl functionality is

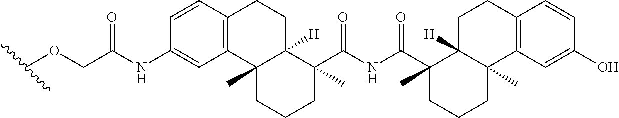 [SMILES: *OCC(=O)Nc1ccc2c(c1)[C@@]1(C)CCC[C@](C)(C(=O)NC(=O)[C@@]3(C)CCC[C@]4(C)c5cc(O)ccc5CC[C@@H]34)[C@@H]1CC2]

wherein [symbol] indicates attachment to a prodrug moiety (as shown in Formulae Ia and/or Ib), a linker, and/or binding agent, as described herein. In one embodiment, the biologically active compound (D*) including hydroxyl functionality is an LXR agonist, and the residue including the hydroxyl functionality is

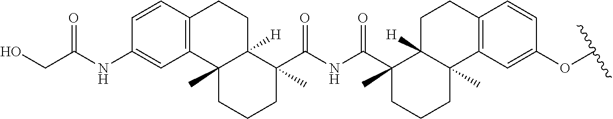 [SMILES: *Oc1ccc2c(c1)[C@@]1(C)CCC[C@](C)(C(=O)NC(=O)[C@@]3(C)CCC[C@]4(C)c5cc(NC(=O)CO)ccc5CC[C@@H]34)[C@@H]1CC2]

wherein [symbol] indicates attachment to a prodrug moiety (as shown in Formulae Ia and/or Ib), a linker, and/or binding agent, as described herein. In certain embodiments, the biologically active compound (D*) or residue thereof includes amino functionality (e.g., D*-NR₂ or D*-N(R)—R). In one embodiment, the amino functionality is a primary amino moiety (e.g., D*-CH₂NR₂ or D*-CH₂N(R)—R; or D*-C(O)CH₂NR₂ or D*-C(O)CH₂N(R)—R). In another embodiment, the amino functionality is a secondary amino moiety (e.g., D*-CH(NR₂)R or D*-CH(NR—R)R; or D*-C(O)CH(R)(NR₂) or D*-C(O)CH(R)(NR—R)). In another embodiment, the amino functionality is a tertiary amino moiety (e.g., D*-C(R₁)(R₂)(NR₂) or D*-C(R₁)(R₂)(N(R)—R); or D*-C(O)C(R₁)(R₂)(NR₂) or D*-C(O)C(R₁)(R₂)(N(R)—R)). In another embodiment, the D* including the amino functionality is an aryl amine (e.g., D*-Ar—NR₂, D*-Ar—N(R)—R. In certain embodiments, the biologically active compound (D*) or residue thereof includes thiol functionality (e.g., D*-SH or D*-S—R). In one embodiment, the thiol functionality is a primary thiol moiety (e.g., D*-CH₂SH or D*-CH₂S—R; or D*-C(O)CH₂SH or D*-C(O)CH₂S—R). In another embodiment, the thiol functionality is a secondary thiol moiety (e.g., D*-CH(SH)R or D*-CH(S—R)R; or D*-C(O)CH(R)(SH) or D*-C(O)CH(R)(S—R)). In another embodiment, the thiol functionality is a tertiary thiol moiety (e.g., D*-C(R₁)(R₂)(SH) or D*-C(R₁)(R₂)(S—R); or D*-C(O)C(R₁)(R₂)(SH) or D*-C(O)C(R₁)(R₂)(S—R)). In another embodiment, the D* including the thiol functionality is an aryl thiol or thiophenol (e.g., D*-Ar-SH, D*-Ar—S—R. In certain embodiments, D* is a tetra- or penta-cyclic steroidal scaffold, as would be appreciated by a person of skill in the art. In certain embodiments, the compounds can be delivered to cells as part of a conjugate. In certain embodiments, the compounds are capable of carrying out any activity of steroids, steroid derivatives, LXR modulators, or derivatives thereof at or in a target, for instance, a target cell. Certain compounds can have one or more additional activities.

In certain embodiments, set forth herein is a compound having the structure of Formula Ia:

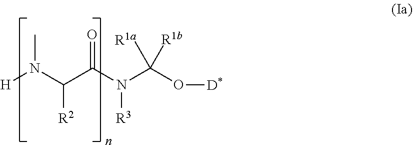

or a pharmaceutically acceptable salt thereof, wherein R^{1a} and R^{1b} are, independently, hydrogen, alkyl, alkoxy, alkenyl, alkynyl, aryl, arylalkyl, heteroaryl, or alkylene, wherein when R^{1a} is alkylene, the alkylene is further bonded to R³ to form a 4-, 5-, or 6-membered heterocyclyl; R² is hydrogen or an amino acid side chain; R³ is hydrogen, alkyl, or alkylene, wherein when R³ is alkylene, the alkylene is further bonded to R^{1a} to form the 4-, 5-, or 6-membered heterocyclyl; D* is acyl, or a residue of a biologically active compound comprising hydroxyl; and n is zero, one, two, three, four, or five.

In certain embodiments, set forth herein is a compound having a structure of Formula Iaa:

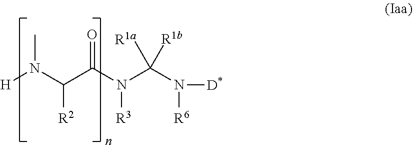

or a pharmaceutically acceptable salt thereof, wherein R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; R⁶ is hydrogen or alkyl; D* is acyl, or a residue of a biologically active compound comprising amino; and n is zero, one, two, three, four, or five.

In certain embodiments, set forth herein is a compound having a structure of Formula Iaaa:

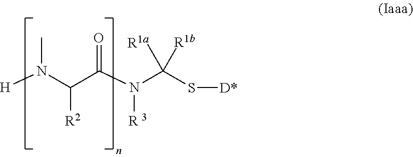

or a pharmaceutically acceptable salt thereof, wherein R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; D* is acyl, or a residue of a biologically active compound comprising thiol; and n is zero, one, two, three, four, or five.

In certain embodiments, set forth herein is a compound having the structure of Formula Ib:

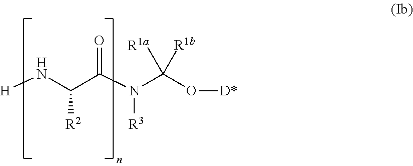

or a pharmaceutically acceptable salt thereof, wherein R^{1a} and R^{1b} are, independently, hydrogen, alkyl, alkoxy, alkenyl, alkynyl, aryl, arylalkyl, heteroaryl, or alkylene, wherein when R^{1a} is alkylene, the alkylene is further bonded to R³ to form a 4-, 5-, or 6-membered heterocyclyl; R² is hydrogen or an amino acid side chain; R³ is hydrogen, alkyl, or alkylene, wherein when R³ is alkylene, the alkylene is further bonded to R^{1a} to form the 4-, 5-, or 6-membered heterocyclyl; D* is acyl, or a residue of a biologically active compound comprising hydroxyl; and n is zero, one, two, three, four, or five.

In certain embodiments, set forth herein is a compound having the structure of Formula Ibb:

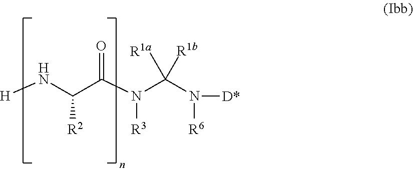

or a pharmaceutically acceptable salt thereof, wherein R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; R⁶ is hydrogen or alkyl; D* is acyl, or a residue of a biologically active compound comprising amino; and n is zero, one, two, three, four, or five.

In certain embodiments, set forth herein is a compound having the structure of Formula Ibbb:

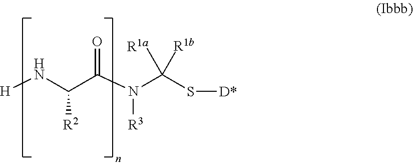

or a pharmaceutically acceptable salt thereof, wherein R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; D* is acyl, or a residue of a biologically active compound comprising thiol; and n is zero, one, two, three, four, or five.

The following embodiments of Formula I and/or Formula Ib are contemplated, where in any one or more of the foregoing embodiments, the biologically active compound includes hydroxyl; or in a residue of a biologically active compound, D*, in certain embodiments, is linked to the rest of the molecule through a residue of the hydroxyl (i.e., a bond to the oxygen). In one embodiment, R^{1a} is hydrogen and R^{1b} is hydrogen; R² is hydrogen; R³ is hydrogen; D* is hydrogen; and n is zero. In another embodiment, R^{1a} is hydrogen and R^{1b} is alkyl; R² is hydrogen; R³ is hydrogen; D* is hydrogen; and n is zero. In another embodiment, R^{1a} is hydrogen and R^{1b} is alkoxy; R² is hydrogen; R³ is hydrogen; D* is hydrogen; and n is zero. In another embodiment, R^{1a} is hydrogen and R^{1b} is alkenyl; R² is hydrogen; R³ is hydrogen; D* is hydrogen; and n is zero. In another embodiment, R^{1a} is hydrogen and R^{1b} is alkynyl; R² is hydrogen; R³ is hydrogen; D* is hydrogen; and n is zero. In another embodiment, R^{1a} is hydrogen and R^{1b} is aryl; R² is hydrogen; R³ is hydrogen; D* is hydrogen; and n is zero. In another embodiment, R^{1a} is hydrogen and R^{1b} is arylalkyl; R² is hydrogen; R³ is hydrogen; D* is hydrogen; and n is zero. In another embodiment, R^{1a} is hydrogen and R^{1b} is heteroaryl; R² is hydrogen; R³ is hydrogen; D* is hydrogen; and n is zero. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is hydrogen; and n is zero. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is hydrogen; and n is zero. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is hydrogen; and n is zero. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is hydrogen; and n is zero. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is hydrogen; and n is zero. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is hydrogen; and n is zero. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is hydrogen; and n is zero. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is hydrogen; and n is zero. In another embodiment, R^{1a} is hydrogen and R^{1b} is hydrogen; R² is an amino acid side chain; R³ is hydrogen; D* is hydrogen; and n is zero. In another embodiment, R^{1a} is hydrogen and R^{1b} is alkyl; R² is amino acid side chain; R³ is hydrogen; D* is hydrogen; and n is zero. In another embodiment, R^{1a} is hydrogen and R^{1b} is alkoxy; R² is an amino acid side chain; R³ is hydrogen; D* is hydrogen; and n is zero. In another embodiment, R^{1a} is hydrogen and R^{1b} is alkenyl; R² is an amino acid side chain; R³ is hydrogen; D* is hydrogen; and n is zero. In another embodiment, R^{1a} is hydrogen and R^{1b} is alkynyl; R² is an amino acid side chain; R³ is hydrogen; D* is hydrogen; and n is zero. In another embodiment, R^{1a} is hydrogen and R^{1b} is aryl; R² is an amino acid side chain; R³ is hydrogen; D* is hydrogen; and n is zero. In another embodiment, R^{1a} is hydrogen and R^{1b} is arylalkyl; R² is an amino acid side chain; R³ is hydrogen; D* is hydrogen; and n is zero. In another embodiment, R^{1a} is hydrogen and R^{1b} is heteroaryl; R² is an amino acid side chain; R³ is hydrogen; D* is hydrogen; and n is zero. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is hydrogen; and n is zero. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is hydrogen; and n is zero. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is hydrogen; and n is zero. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is hydrogen; and n is zero. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is hydrogen; and n is zero. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is hydrogen; and n is zero. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is hydrogen; and n is zero. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is hydrogen; and n is zero. In another embodiment, R^{1a} is hydrogen and R^{1b} is hydrogen; R² is hydrogen; R³ is alkyl; D* is hydrogen; and n is zero. In another embodiment, R^{1a} is hydrogen and R^{1b} is alkyl; R² is hydrogen; R³ is alkyl; D* is hydrogen; and n is zero. In another embodiment, R^{1a} is hydrogen and R^{1b} is alkoxy; R² is hydrogen; R³ is alkyl; D* is hydrogen; and n is zero. In another embodiment, R^{1a} is hydrogen and R^{1b} is alkenyl; R² is hydrogen; R³ is alkyl; D* is hydrogen; and n is zero. In another embodiment, R^{1a} is hydrogen and R^{1b} is alkynyl; R² is hydrogen; R³ is alkyl; D* is hydrogen; and n is zero. In another embodiment, R^{1a} is hydrogen and R^{1b} is aryl; R² is hydrogen; R³ is alkyl; D* is hydrogen; and n is zero. In another embodiment, R^{1a} is hydrogen and R^{1b} is arylalkyl; R² is hydrogen; R³ is alkyl; D* is hydrogen; and n is zero. In another embodiment, R^{1a} is hydrogen and R^{1b} is heteroaryl; R² is hydrogen; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is hydrogen; and n is zero. In another embodiment, R^{1a} is hydrogen and R^{1b} is hydrogen; R² is an amino acid side chain; R³ is alkyl; D* is hydrogen; and n is zero. In another embodiment, R^{1a} is hydrogen and R^{1b} is alkyl; R² is amino acid side chain; R³ is alkyl; D* is hydrogen; and n is zero. In any of the embodiments in this paragraph, R⁴ is hydrogen. In any of the embodiments in this paragraph, R⁴ is alkyl.

In another embodiment, R^{1a} is hydrogen and R^{1b} is alkoxy; R² is an amino acid side chain; R³ is alkyl; D* is hydrogen; and n is zero. In another embodiment, R^{1a} is hydrogen and R^{1b} is alkenyl; R² is an amino acid side chain; R³ is alkyl; D* is hydrogen; and n is zero. In another embodiment, R^{1a} is hydrogen and R^{1b} is alkynyl; R² is an amino acid side chain; R³ is alkyl; D* is hydrogen; and n is zero. In another embodiment, R^{1a} is hydrogen and R^{1b} is aryl; R² is an amino acid side chain; R³ is alkyl; D* is hydrogen; and n is zero. In another embodiment, R^{1a} is hydrogen and R^{1b} is arylalkyl; R² is an amino acid side chain; R³ is alkyl; D* is hydrogen; and n is zero. In another embodiment, R^{1a} is hydrogen and R^{1b} is heteroaryl; R² is an amino acid side chain; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is hydrogen; and n is zero. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is hydrogen; and n is zero. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is hydrogen; and n is zero. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is hydrogen; and n is zero. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4-, 5-, or 6-membered heterocyclyl; D* is hydrogen; and n is zero. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is hydrogen; and n is zero. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is hydrogen; and n is zero. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is hydrogen; and n is zero. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is hydrogen; and n is zero. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is hydrogen; and n is zero. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is hydrogen; and n is zero. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is hydrogen; and n is zero. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is hydrogen; and n is zero. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is hydrogen; and n is zero. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is hydrogen; and n is zero. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is hydrogen; and n is zero. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is hydrogen; and n is zero. In another embodiment, R^{1a} is hydrogen and R^{1b} is hydrogen; R² is hydrogen; R³ is hydrogen; D* is acyl; and n is zero. In another embodiment, R^{1a} is hydrogen and R^{1b} is alkyl; R² is hydrogen; R³ is hydrogen; D* is acyl; and n is zero. In another embodiment, R^{1a} is hydrogen and R^{1b} is alkoxy; R² is hydrogen; R³ is hydrogen; D* is acyl; and n is zero. In another embodiment, R^{1a} is hydrogen and R^{1b} is alkenyl; R² is hydrogen; R³ is hydrogen; D* is acyl; and n is zero. In another embodiment, R^{1a} is hydrogen and R^{1b} is alkynyl; R² is hydrogen; R³ is hydrogen; D* is acyl; and n is zero. In another embodiment, R^{1a} is hydrogen and R^{1b} is aryl; R² is hydrogen; R³ is hydrogen; D* is acyl; and n is zero. In another embodiment, R^{1a} is hydrogen and R^{1b} is arylalkyl; R² is hydrogen; R³ is hydrogen; D* is acyl; and n is zero. In another embodiment, R^{1a} is hydrogen and R^{1b} is heteroaryl; R² is hydrogen; R³ is hydrogen; D* is acyl; and n is zero. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is acyl; and n is zero. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is acyl; and n is zero. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is acyl; and n is zero. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is acyl; and n is zero. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is acyl; and n is zero. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is acyl; and n is zero. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is acyl; and n is zero. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is acyl; and n is zero. In another embodiment, R^{1a} is hydrogen and R^{1b} is hydrogen; R² is an amino acid side chain; R³ is hydrogen; D* is acyl; and n is zero. In another embodiment, R^{1a} is hydrogen and R^{1b} is alkyl; R² is amino acid side chain; R³ is hydrogen; D* is acyl; and n is zero. In another embodiment, R^{1a} is hydrogen and R^{1b} is alkoxy; R² is an amino acid side chain; R³ is hydrogen; D* is acyl; and n is zero. In another embodiment, R^{1a} is hydrogen and R^{1b} is alkenyl; R² is an amino acid side chain; R³ is hydrogen; D* is acyl; and n is zero. In another embodiment, R^{1a} is hydrogen and R^{1b} is alkynyl; R² is an amino acid side chain; R³ is hydrogen; D* is acyl; and n is zero. In another embodiment, R^{1a} is hydrogen and R^{1b} is aryl; R² is an amino acid side chain; R³ is hydrogen; D* is acyl; and n is zero. In another embodiment, R^{1a} is hydrogen and R^{1b} is arylalkyl; R² is an amino acid side chain; R³ is hydrogen; D* is acyl; and n is zero. In another embodiment, R^{1a} is hydrogen and R^{1b} is heteroaryl; R² is an amino acid side chain; R³ is hydrogen; D* is acyl; and n is zero. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is acyl; and n is zero. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is acyl; and n is zero. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is acyl; and n is zero. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is acyl; and n is zero. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is acyl; and n is zero. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is acyl; and n is zero. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is acyl; In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is acyl; and n is zero. In another embodiment, R^{1a} is hydrogen and R^{1b} is hydrogen; R² is hydrogen; R³ is alkyl; D* is acyl; and n is zero. In another embodiment, R^{1a} is hydrogen and R^{1b} is alkyl; R² is hydrogen; R³ is alkyl; D* is acyl; and n is zero. In another embodiment, R^{1a} is hydrogen and R^{1b} is alkoxy; R² is hydrogen; R³ is alkyl; D* is acyl; and n is zero. In another embodiment, R^{1a} is hydrogen and R^{1b} is alkenyl; R² is hydrogen; R³ is alkyl; D* is acyl; and n is zero. In another embodiment, R^{1a} is hydrogen and R^{1b} is alkynyl; R² is hydrogen; R³ is alkyl; D* is acyl; and n is zero. In another embodiment, R^{1a} is hydrogen and R^{1b} is aryl; R² is hydrogen; R³ is alkyl; D* is acyl; and n is zero. In another embodiment, R^{1a} is hydrogen and R^{1b} is arylalkyl; R² is hydrogen; R³ is alkyl; D* is acyl; and n is zero. In another embodiment, R^{1a} is hydrogen and R^{1b} is heteroaryl; R² is hydrogen; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is acyl; and n is zero. In another embodiment, R^{1a} is hydrogen and R^{1b} is hydrogen; R² is an amino acid side chain; R³ is alkyl; D* is acyl; and n is zero. In another embodiment, R^{1a} is hydrogen and R^{1b} is alkyl; R² is amino acid side chain; R³ is alkyl; D* is acyl; and n is zero. In any of the embodiments in this paragraph, R⁴ is hydrogen. In any of the embodiments in this paragraph, R⁴ is alkyl.

In another embodiment, R^{1a} is hydrogen and R^{1b} is alkoxy; R² is an amino acid side chain; R³ is alkyl; D* is acyl; and n is zero. In another embodiment, R^{1a} is hydrogen and R^{1b} is alkenyl; R² is an amino acid side chain; R³ is alkyl; D* is acyl; and n is zero. In another embodiment, R^{1a} is hydrogen and R^{1b} is alkynyl; R² is an amino acid side chain; R³ is alkyl; D* is acyl; and n is zero. In another embodiment, R^{1a} is hydrogen and R^{1b} is aryl; R² is an amino acid side chain; R³ is alkyl; D* is acyl; and n is zero. In another embodiment, R^{1a} is hydrogen and R^{1b} is arylalkyl; R² is an amino acid side chain; R³ is alkyl; D* is acyl; and n is zero. In another embodiment, R^{1a} is hydrogen and R^{1b} is heteroaryl; R² is an amino acid side chain; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is acyl; and n is zero. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is acyl; and n is zero. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is acyl; and n is zero. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is acyl; and n is zero. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4, 5-, or 6-membered heterocyclyl; D* is acyl; and n is zero. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is acyl; and n is zero. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is acyl; and n is zero. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is acyl; and n is zero. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is acyl; and n is zero. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is acyl; and n is zero. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is acyl; and n is zero. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is acyl; and n is zero. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is acyl; and n is zero. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is acyl; and n is zero. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is acyl; and n is zero. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is acyl; and n is zero. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is acyl; and n is zero. In another embodiment, R^{1a} is hydrogen and R^{1b} is hydrogen; R² is hydrogen; R³ is hydrogen; D* is a residue of a biologically active compound; and n is zero. In another embodiment, R^{1a} is hydrogen and R^{1b} is alkyl; R² is hydrogen; R³ is hydrogen; D* is a residue of a biologically active compound; and n is zero. In another embodiment, R^{1a} is hydrogen and R^{1b} is alkoxy; R² is hydrogen; R³ is hydrogen; D* is a residue of a biologically active compound; and n is zero. In another embodiment, R^{1a} is hydrogen and R^{1b} is alkenyl; R² is hydrogen; R³ is hydrogen; D* is a residue of a biologically active compound; and n is zero. In another embodiment, R^{1a} is hydrogen and R^{1b} is alkynyl; R² is hydrogen; R³ is hydrogen; D* is a residue of a biologically active compound; and n is zero. In another embodiment, R^{1a} is hydrogen and R^{1b} is aryl; R² is hydrogen; R³ is hydrogen; D* is a residue of a biologically active compound; and n is zero. In another embodiment, R^{1a} is hydrogen and R^{1b} is arylalkyl; R² is hydrogen; R³ is hydrogen; D* is a residue of a biologically active compound; and n is zero. In another embodiment, R^{1a} is hydrogen and R^{1b} is heteroaryl; R² is hydrogen; R³ is hydrogen; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is a residue of a biologically active compound; and n is zero. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is a residue of a biologically active compound; and n is zero. In another embodiment, R^{1a} is hydrogen and R^{1b} is hydrogen; R² is an amino acid side chain; R³ is hydrogen; D* is a residue of a biologically active compound; and n is zero. In another embodiment, R^{1a} is hydrogen and R^{1b} is alkyl; R² is amino acid side chain; R³ is hydrogen; D* is a residue of a biologically active compound; and n is zero. In another embodiment, R^{1a} is hydrogen and R^{1b} is alkoxy; R² is an amino acid side chain; R³ is hydrogen; D* is a residue of a biologically active compound; and n is zero. In another embodiment, R^{1a} is hydrogen and R^{1b} is alkenyl; R² is an amino acid side chain; R³ is hydrogen; D* is a residue of a biologically active compound; and n is zero. In another embodiment, R^{1a} is hydrogen and R^{1b} is alkynyl; R² is an amino acid side chain; R³ is hydrogen; D* is a residue of a biologically active compound; and n is zero. In another embodiment, R^{1a} is hydrogen and R^{1b} is aryl; R² is an amino acid side chain; R³ is hydrogen; D* is a residue of a biologically active compound; and n is zero. In another embodiment, R^{1a} is hydrogen and R^{1b} is arylalkyl; R² is an amino acid side chain; R³ is hydrogen; D* is a residue of a biologically active compound; and n is zero. In another embodiment, R^{1a} is hydrogen and R^{1b} is heteroaryl; R² is an amino acid side chain; R³ is hydrogen; D* is a residue of a biologically active compound; and n is zero. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is a residue of a biologically active compound; and n is zero. In another embodiment, R^{1a} is hydrogen and R^{1b} is hydrogen; R² is hydrogen; R³ is alkyl; D* is a residue of a biologically active compound; and n is zero. In another embodiment, R^{1a} is hydrogen and R^{1b} is alkyl; R² is hydrogen; R³ is alkyl; D* is a residue of a biologically active compound; and n is zero. In another embodiment, R^{1a} is hydrogen and R^{1b} is alkoxy; R² is hydrogen; R³ is alkyl; D* is a residue of a biologically active compound; and n is zero. In another embodiment, R^{1a} is hydrogen and R^{1b} is alkenyl; R² is hydrogen; R³ is alkyl; D* is a residue of a biologically active compound; and n is zero. In another embodiment, R^{1a} is hydrogen and R^{1b} is alkynyl; R² is hydrogen; R³ is alkyl; D* is a residue of a biologically active compound; and n is zero. In another embodiment, R^{1a} is hydrogen and R^{1b} is aryl; R² is hydrogen; R³ is alkyl; D* is a residue of a biologically active compound; and n is zero. In any of the embodiments in this paragraph, R⁴ is hydrogen. In any of the embodiments in this paragraph, R⁴ is alkyl.

In another embodiment, R^{1a} is hydrogen and R^{1b} is arylalkyl; R² is hydrogen; R³ is alkyl; D* is a residue of a biologically active compound; and n is zero. In another embodiment, R^{1a} is hydrogen and R^{1b} is heteroaryl; R² is hydrogen; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is a residue of a biologically active compound; and n is zero. In another embodiment, R^{1a} is hydrogen and R^{1b} is hydrogen; R² is an amino acid side chain; R³ is alkyl; D* is a residue of a biologically active compound; and n is zero. In another embodiment, R^{1a} is hydrogen and R^{1b} is alkyl; R² is amino acid side chain; R³ is alkyl; D* is a residue of a biologically active compound; and n is zero. In another embodiment, R^{1a} is hydrogen and R^{1b} is alkoxy; R² is an amino acid side chain; R³ is alkyl; D* is a residue of a biologically active compound; and n is zero. In another embodiment, R^{1a} is hydrogen and R^{1b} is alkenyl; R² is an amino acid side chain; R³ is alkyl; D* is a residue of a biologically active compound; and n is zero. In any of the embodiments in this paragraph, R⁴ is hydrogen. In any of the embodiments in this paragraph, R⁴ is alkyl.

In another embodiment, R^{1a} is hydrogen and R^{1b} is alkynyl; R² is an amino acid side chain; R³ is alkyl; D* is a residue of a biologically active compound; and n is zero. In another embodiment, R^{1a} is hydrogen and R^{1b} is aryl; R² is an amino acid side chain; R³ is alkyl; D* is a residue of a biologically active compound; and n is zero. In another embodiment, R^{1a} is hydrogen and R^{1b} is arylalkyl; R² is an amino acid side chain; R³ is alkyl; D* is a residue of a biologically active compound; and n is zero. In another embodiment, R^{1a} is hydrogen and R^{1b} is heteroaryl; R² is an amino acid side chain; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is a residue of a biologically active compound; and n is zero. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4-, 5-, or 6-membered heterocyclyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is a residue of a biologically active compound; and n is zero. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is a residue of a biologically active compound; and n is zero. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5, or 6-membered heterocyclyl; D* is a residue of a biologically active compound; and n is zero. In any of the embodiments in this paragraph, R⁴ is hydrogen. In any of the embodiments in this paragraph, R⁴ is alkyl.

In another embodiment, R^{1a} is hydrogen and R^{1b} is hydrogen; R² is hydrogen; R³ is hydrogen; D* is hydrogen; and n is one. In another embodiment, R^{1a} is hydrogen and R^{1b} is alkyl; R² is hydrogen; R³ is hydrogen; D* is hydrogen; and n is one. In another embodiment, R^{1a} is hydrogen and R^{1b} is alkoxy; R² is hydrogen; R³ is hydrogen; D* is hydrogen; and n is one. In another embodiment, R^{1a} is hydrogen and R^{1b} is alkenyl; R² is hydrogen; R³ is hydrogen; D* is hydrogen; and n is one. In another embodiment, R^{1a} is hydrogen and R^{1b} is alkynyl; R² is hydrogen; R³ is hydrogen; D* is hydrogen; and n is one. In another embodiment, R^{1a} is hydrogen and R^{1b} is aryl; R² is hydrogen; R³ is hydrogen; D* is hydrogen; and n is one. In another embodiment, R^{1a} is hydrogen and R^{1b} is arylalkyl; R² is hydrogen; R³ is hydrogen; D* is hydrogen; and n is one. In another embodiment, R^{1a} is hydrogen and R^{1b} is heteroaryl; R² is hydrogen; R³ is hydrogen; D* is hydrogen; and n is one. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is hydrogen; and n is one. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is hydrogen; and n is one. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is hydrogen; and n is one. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is hydrogen; and n is one. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is hydrogen; and n is one. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is hydrogen; and n is one. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is hydrogen; and n is one. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is hydrogen; and n is one. In another embodiment, R^{1a} is hydrogen and R^{1b} is hydrogen; R² is an amino acid side chain; R³ is hydrogen; D* is hydrogen; and n is one. In another embodiment, R^{1a} is hydrogen and R^{1b} is alkyl; R² is amino acid side chain; R³ is hydrogen; D* is hydrogen; and n is one. In another embodiment, R^{1a} is hydrogen and R^{1b} is alkoxy; R² is an amino acid side chain; R³ is hydrogen; D* is hydrogen; and n is one. In another embodiment, R^{1a} is hydrogen and R^{1b} is alkenyl; R² is an amino acid side chain; R³ is hydrogen; D* is hydrogen; and n is one. In another embodiment, R^{1a} is hydrogen and R^{1b} is alkynyl; R² is an amino acid side chain; R³ is hydrogen; D* is hydrogen; and n is one. In another embodiment, R^{1a} is hydrogen and R^{1b} is aryl; R² is an amino acid side chain; R³ is hydrogen; D* is hydrogen; and n is one. In another embodiment, R^{1a} is hydrogen and R^{1b} is arylalkyl; R² is an amino acid side chain; R³ is hydrogen; D* is hydrogen; and n is one. In another embodiment, R^{1a} is hydrogen and R^{1b} is heteroaryl; R² is an amino acid side chain; R³ is hydrogen; D* is hydrogen; and n is one. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is hydrogen; and n is one. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is hydrogen; and n is one. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is hydrogen; and n is one. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is hydrogen; and n is one. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is hydrogen; and n is one. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is hydrogen; and n is one. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is hydrogen; and n is one. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is hydrogen; and n is one. In another embodiment, R^{1a} is hydrogen and R^{1b} is hydrogen; R² is hydrogen; R³ is alkyl; D* is hydrogen; and n is one. In another embodiment, R^{1a} is hydrogen and R^{1b} is alkyl; R² is hydrogen; R³ is alkyl; D* is hydrogen; and n is one. In another embodiment, R^{1a} is hydrogen and R^{1b} is alkoxy; R² is hydrogen; R³ is alkyl; D* is hydrogen; and n is one. In another embodiment, R^{1a} is hydrogen and R^{1b} is alkenyl; R² is hydrogen; R³ is alkyl; D* is hydrogen; and n is one. In another embodiment, R^{1a} is hydrogen and R^{1b} is alkynyl; R² is hydrogen; R³ is alkyl; D* is hydrogen; and n is one. In another embodiment, R^{1a} is hydrogen and R^{1b} is aryl; R² is hydrogen; R³ is alkyl; D* is hydrogen; and n is one. In another embodiment, R^{1a} is hydrogen and R^{1b} is arylalkyl; R² is hydrogen; R³ is alkyl; D* is hydrogen; and n is one. In another embodiment, R^{1a} is hydrogen and R^{1b} is heteroaryl; R² is hydrogen; R³ is alkyl; D* is hydrogen; and n is one. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is hydrogen; and n is one. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is hydrogen; and n is one. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is hydrogen; and n is one. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is hydrogen; and n is one. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is hydrogen; and n is one. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is hydrogen; and n is one. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is hydrogen; and n is one. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is hydrogen; and n is one. In another embodiment, R^{1a} is hydrogen and R^{1b} is hydrogen; R² is an amino acid side chain; R³ is alkyl; D* is hydrogen; and n is one. In another embodiment, R^{1a} is hydrogen and R^{1b} is alkyl; R² is amino acid side chain; R³ is alkyl; D* is hydrogen; and n is one. In another embodiment, R^{1a} is hydrogen and R^{1b} is alkoxy; R² is an amino acid side chain; R³ is alkyl; D* is hydrogen; and n is one. In another embodiment, R^{1a} is hydrogen and R^{1b} is alkenyl; R² is an amino acid side chain; R³ is alkyl; D* is hydrogen; and n is one. In another embodiment, R^{1a} is hydrogen and R^{1b} is alkynyl; R² is an amino acid side chain; R³ is alkyl; D* is hydrogen; and n is one. In another embodiment, R^{1a} is hydrogen and R^{1b} is aryl; R² is an amino acid side chain; R³ is alkyl; D* is hydrogen; and n is one. In another embodiment, R^{1a} is hydrogen and R^{1b} is arylalkyl; R² is an amino acid side chain; R³ is alkyl; D* is hydrogen; and n is one. In another embodiment, R^{1a} is hydrogen and R^{1b} is heteroaryl; R² is an amino acid side chain; R³ is alkyl; D* is hydrogen; and n is one. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is hydrogen; and n is one. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is hydrogen; and n is one. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is hydrogen; and n is one. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is hydrogen; and n is one. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is hydrogen; and n is one. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is hydrogen; and n is one. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is hydrogen; and n is one. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is hydrogen; and n is one. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is hydrogen; and n is one. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is hydrogen; and n is one. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene, further bonded to R^{1a} to form the 4-, 5-, or 6-membered heterocyclyl; D* is hydrogen; and n is one. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4-, 5-, or 6-membered heterocyclyl; D* is hydrogen; and n is one. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is hydrogen; and n is one. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is hydrogen; and n is one. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is hydrogen; and n is one. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is hydrogen; and n is one. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is hydrogen; and n is one. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is hydrogen; and n is one. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is hydrogen; and n is one. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is hydrogen; and n is one. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is hydrogen; and n is one. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is hydrogen; and n is one. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is hydrogen; and n is one. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is hydrogen; and n is one. In another embodiment, R^{1a} is hydrogen and R^{1b} is hydrogen; R² is hydrogen; R³ is hydrogen; D* is acyl; and n is one. In another embodiment, R^{1a} is hydrogen and R^{1b} is alkyl; R² is hydrogen; R³ is hydrogen; D* is acyl; and n is one. In another embodiment, R^{1a} is hydrogen and R^{1b} is alkoxy; R² is hydrogen; R³ is hydrogen; D* is acyl; and n is one. In another embodiment, Ria is hydrogen and R^{1b} is alkenyl; R² is hydrogen; R³ is hydrogen; D* is acyl; and n is one. In another embodiment, R^{1a} is hydrogen and R^{1b} is alkynyl; R² is hydrogen; R³ is hydrogen; D* is acyl; and n is one. In another embodiment, R^{1a} is hydrogen and R^{1b} is aryl; R² is hydrogen; R³ is hydrogen; D* is acyl; and n is one. In another embodiment, R^{1a} is hydrogen and R^{1b} is arylalkyl; R² is hydrogen; R³ is hydrogen; D* is acyl; and n is one. In another embodiment, R^{1a} is hydrogen and R^{1b} is heteroaryl; R² is hydrogen; R³ is hydrogen; D* is acyl; and n is one. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is acyl; and n is one. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is acyl; and n is one. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is acyl; and n is one. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is acyl; and n is one. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is acyl; and n is one. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is acyl; and n is one. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is acyl; and n is one. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is acyl; and n is one. In another embodiment, R^{1a} is hydrogen and R^{1b} is hydrogen; R² is an amino acid side chain; R³ is hydrogen; D* is acyl; and n is one. In another embodiment, R^{1a} is hydrogen and R^{1b} is alkyl; R² is amino acid side chain; R³ is hydrogen; D* is acyl; and n is one. In another embodiment, R^{1a} is hydrogen and R^{1b} is alkoxy; R² is an amino acid side chain; R³ is hydrogen; D* is acyl; and n is one. In another embodiment, R^{1a} is hydrogen and R^{1b} is alkenyl; R² is an amino acid side chain; R³ is hydrogen; D* is acyl; and n is one. In another embodiment, R^{1a} is hydrogen and R^{1b} is alkynyl; R² is an amino acid side chain; R³ is hydrogen; D* is acyl; and n is one. In another embodiment, R^{1a} is hydrogen and R^{1b} is aryl; R² is an amino acid side chain; R³ is hydrogen; D* is acyl; and n is one. In another embodiment, R^{1a} is hydrogen and R^{1b} is arylalkyl; R² is an amino acid side chain; R³ is hydrogen; D* is acyl; and n is one. In another embodiment, R^{1a} is hydrogen and R^{1b} is heteroaryl; R² is an amino acid side chain; R³ is hydrogen; D* is acyl; and n is one. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is acyl; and n is one. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is acyl; and n is one. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is acyl; and n is one. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is acyl; and n is one. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is acyl; and n is one. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is acyl; and n is one. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is acyl; and n is one. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is acyl; and n is one. In another embodiment, R^{1a} is hydrogen and R^{1b} is hydrogen; R² is hydrogen; R³ is alkyl; D* is acyl; and n is one. In another embodiment, R^{1a} is hydrogen and R^{1b} is alkyl; R² is hydrogen; R³ is alkyl; D* is acyl; and n is one. In another embodiment, R^{1a} is hydrogen and R^{1b} is alkoxy; R² is hydrogen; R³ is alkyl; D* is acyl; and n is one. In another embodiment, R^{1a} is hydrogen and R^{1b} is alkenyl; R² is hydrogen; R³ is alkyl; D* is acyl; and n is one. In another embodiment, R^{1a} is hydrogen and R^{1b} is alkynyl; R² is hydrogen; R³ is alkyl; D* is acyl; and n is one. In another embodiment, R^{1a} is hydrogen and R^{1b} is aryl; R² is hydrogen; R³ is alkyl; D* is acyl; and n is one. In another embodiment, R^{1a} is hydrogen and R^{1b} is arylalkyl; R² is hydrogen; R³ is alkyl; D* is acyl; and n is one. In another embodiment, R^{1a} is hydrogen and R^{1b} is heteroaryl; R² is hydrogen; R³ is alkyl; D* is acyl; and n is one. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is acyl; and n is one. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is acyl; and n is one. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is acyl; and n is one. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is acyl; and n is one. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is acyl; and n is one. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is acyl; and n is one. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is acyl; and n is one. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is acyl; and n is one. In another embodiment, R^{1a} is hydrogen and R^{1b} is hydrogen; R² is an amino acid side chain; R³ is alkyl; D* is acyl; and n is one. In another embodiment, R^{1a} is hydrogen and R^{1b} is alkyl; R² is amino acid side chain; R³ is alkyl; D* is acyl; and n is one. In another embodiment, R^{1a} is hydrogen and R^{1b} is alkoxy; R² is an amino acid side chain; R³ is alkyl; D* is acyl; and n is one. In another embodiment, R^{1a} is hydrogen and R^{1b} is alkenyl; R² is an amino acid side chain; R³ is alkyl; D* is acyl; and n is one. In another embodiment, R^{1a} is hydrogen and R^{1b} is alkynyl; R² is an amino acid side chain; R³ is alkyl; D* is acyl; and n is one. In another embodiment, R^{1a} is hydrogen and R^{1b} is aryl; R² is an amino acid side chain; R³ is alkyl; D* is acyl; and n is one. In another embodiment, R^{1a} is hydrogen and R^{1b} is arylalkyl; R² is an amino acid side chain; R³ is alkyl; D* is acyl; and n is one. In another embodiment, R^{1a} is hydrogen and R^{1b} is heteroaryl; R² is an amino acid side chain; R³ is alkyl; D* is acyl; and n is one. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is acyl; and n is one. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is acyl; and n is one. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is acyl; and n is one. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is acyl; and n is one. In any of the embodiments in this paragraph, R⁴ is hydrogen. In any of the embodiments in this paragraph, R⁴ 15 alkyl.

In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is acyl; and n is one. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is acyl; and n is one. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is acyl; and n is one. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is acyl; and n is one. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is acyl; and n is one. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is acyl; and n is one. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is acyl; and n is one. In another embodiment, Ria is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4, 5-, or 6-membered heterocyclyl; D* is acyl; and n is one. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is acyl; and n is one. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is acyl; and n is one. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is acyl; and n is one. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is acyl; and n is one. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is acyl; and n is one. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is acyl; and n is one. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is acyl; and n is one. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is acyl; and n is one. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is acyl; and n is one. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is acyl; and n is one. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is acyl; and n is one. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is acyl; and n is one. In another embodiment, R^{1a} is hydrogen and R^{1b} is hydrogen; R² is hydrogen; R³ is hydrogen; D* is a residue of a biologically active compound; and n is one. In another embodiment, R^{1a} is hydrogen and R^{1b} is alkyl; R² is hydrogen; R³ is hydrogen; D* is a residue of a biologically active compound; and n is one. In any of the embodiments in this paragraph, R⁴ is hydrogen. In any of the embodiments in this paragraph, R⁴ is alkyl.

In another embodiment, R^{1a} is hydrogen and R^{1b} is alkoxy; R² is hydrogen; R³ is hydrogen; D* is a residue of a biologically active compound; and n is one. In another embodiment, R^{1a} is hydrogen and R^{1b} is alkenyl; R² is hydrogen; R³ is hydrogen; D* is a residue of a biologically active compound; and n is one. In another embodiment, R^{1a} is hydrogen and R^{1b} is alkynyl; R² is hydrogen; R³ is hydrogen; D* is a residue of a biologically active compound; and n is one. In another embodiment, R^{1a} is hydrogen and R^{1b} is aryl; R² is hydrogen; R³ is hydrogen; D* is a residue of a biologically active compound; and n is one. In another embodiment, R^{1a} is hydrogen and R^{1b} is arylalkyl; R² is hydrogen; R³ is hydrogen; D* is a residue of a biologically active compound; and n is one. In another embodiment, R^{1a} is hydrogen and R^{1b} is heteroaryl; R² is hydrogen; R³ is hydrogen; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is a residue of a biologically active compound; and n is one. In any of the embodiments in this paragraph, R⁴ is hydrogen. In any of the embodiments in this paragraph, R⁴ is alkyl.

In another embodiment, R^{1a} is hydrogen and R^{1b} is hydrogen; R² is an amino acid side chain; R³ is hydrogen; D* is a residue of a biologically active compound; and n is one. In another embodiment, R^{1a} is hydrogen and R^{1b} is alkyl; R² is amino acid side chain; R³ is hydrogen; D* is a residue of a biologically active compound; and n is one. In another embodiment, R^{1a} is hydrogen and R^{1b} is alkoxy; R² is an amino acid side chain; R³ is hydrogen; D* is a residue of a biologically active compound; and n is one. In another embodiment, R^{1a} is hydrogen and R^{1b} is alkenyl; R² is an amino acid side chain; R³ is hydrogen; D* is a residue of a biologically active compound; and n is one. In another embodiment, R^{1a} is hydrogen and R^{1b} is alkynyl; R² is an amino acid side chain; R³ is hydrogen; D* is a residue of a biologically active compound; and n is one. In another embodiment, R^{1a} is hydrogen and R^{1b} is aryl; R² is an amino acid side chain; R³ is hydrogen; D* is a residue of a biologically active compound; and n is one. In another embodiment, R^{1a} is hydrogen and R^{1b} is arylalkyl; R² is an amino acid side chain; R³ is hydrogen; D* is a residue of a biologically active compound; and n is one. In another embodiment, R^{1a} is hydrogen and R^{1b} is heteroaryl; R² is an amino acid side chain; R³ is hydrogen; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is a residue of a biologically active compound; and n is one. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is a residue of a biologically active compound; and n is one. In another embodiment, R^{1a} is hydrogen and R^{1b} is hydrogen; R² is hydrogen; R³ is alkyl; D* is a residue of a biologically active compound; and n is one. In another embodiment, R^{1a} is hydrogen and R^{1b} is alkyl; R² is hydrogen; R³ is alkyl; D* is a residue of a biologically active compound; and n is one. In another embodiment, R^{1a} is hydrogen and R^{1b} is alkoxy; R² is hydrogen; R³ is alkyl; D* is a residue of a biologically active compound; and n is one. In another embodiment, R^{1a} is hydrogen and R^{1b} is alkenyl; R² is hydrogen; R³ is alkyl; D* is a residue of a biologically active compound; and n is one. In another embodiment, R^{1a} is hydrogen and R^{1b} is alkynyl; R² is hydrogen; R³ is alkyl; D* is a residue of a biologically active compound; and n is one. In another embodiment, R^{1a} is hydrogen and R^{1b} is aryl; R² is hydrogen; R³ is alkyl; D* is a residue of a biologically active compound; and n is one. In another embodiment, R^{1a} is hydrogen and R^{1b} is arylalkyl; R² is hydrogen; R³ is alkyl; D* is a residue of a biologically active compound; and n is one. In another embodiment, R^{1a} is hydrogen and R^{1b} is heteroaryl; R² is hydrogen; R³ is alkyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is a residue of a biologically active compound; and n is one. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is a residue of a biologically active compound; and n is one. In another embodiment, R^{1a} is hydrogen and R^{1b} is hydrogen; R² is an amino acid side chain; R³ is alkyl; D* is a residue of a biologically active compound; and n is one. In another embodiment, R^{1a} is hydrogen and R^{1b} is alkyl; R² is amino acid side chain; R³ is alkyl; D* is a residue of a biologically active compound; and n is one. In another embodiment, R^{1a} is hydrogen and R^{1b} is alkoxy; R² is an amino acid side chain; R³ is alkyl; D* is a residue of a biologically active compound; and n is one. In another embodiment, R^{1a} is hydrogen and R^{1b} is alkenyl; R² is an amino acid side chain; R³ is alkyl; D* is a residue of a biologically active compound; and n is one. In another embodiment, R^{1a} is hydrogen and R^{1b} is alkynyl; R² is an amino acid side chain; R³ is alkyl; D* is a residue of a biologically active compound; and n is one. In another embodiment, R^{1a} is hydrogen and R^{1b} is aryl; R² is an amino acid side chain; R³ is alkyl; D* is a residue of a biologically active compound; and n is one. In another embodiment, R^{1a} is hydrogen and R^{1b} is arylalkyl; R² is an amino acid side chain; R³ is alkyl; D* is a residue of a biologically active compound; and n is one. In another embodiment, R^{1a} is hydrogen and R^{1b} is heteroaryl; R² is an amino acid side chain; R³ is alkyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is a residue of a biologically active compound; and n is one. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4-, 5-, or 6-membered heterocyclyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is a residue of a biologically active compound; and n is one. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is a residue of a biologically active compound; and n is one. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5, or 6-membered heterocyclyl; D* is a residue of a biologically active compound; and n is one. In any of the embodiments in this paragraph, R⁴ is hydrogen. In any of the embodiments in this paragraph, R⁴ is alkyl.

In another embodiment, R^{1a} is hydrogen and R^{1b} is hydrogen; R² is hydrogen; R³ is hydrogen; D* is hydrogen; and n is two. In another embodiment, R^{1a} is hydrogen and R^{1b} is alkyl; R² is hydrogen; R³ is hydrogen; D* is hydrogen; and n is two. In another embodiment, R^{1a} is hydrogen and R^{1b} is alkoxy; R² is hydrogen; R³ is hydrogen; D* is hydrogen; and n is two. In another embodiment, R^{1a} is hydrogen and R^{1b} is alkenyl; R² is hydrogen; R³ is hydrogen; D* is hydrogen; and n is two. In another embodiment, R^{1a} is hydrogen and R^{1b} is alkynyl; R² is hydrogen; R³ is hydrogen; D* is hydrogen; and n is two. In another embodiment, R^{1a} is hydrogen and R^{1b} is aryl; R² is hydrogen; R³ is hydrogen; D* is hydrogen; and n is two. In another embodiment, R^{1a} is hydrogen and R^{1b} is arylalkyl; R² is hydrogen; R³ is hydrogen; D* is hydrogen; and n is two. In another embodiment, R^{1a} is hydrogen and R^{1b} is heteroaryl; R² is hydrogen; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is hydrogen; and n is two. In another embodiment, R^{1a} is hydrogen and R^{1b} is hydrogen; R² is an amino acid side chain; R³ is hydrogen; D* is hydrogen; and n is two. In another embodiment, R^{1a} is hydrogen and R^{1b} is alkyl; R² is amino acid side chain; R³ is hydrogen; D* is hydrogen; In another embodiment, R^{1a} is hydrogen and R^{1b} is alkoxy; R² is an amino acid side chain; R³ is hydrogen; D* is hydrogen; and n is two. In any of the embodiments in this paragraph, R⁴ is hydrogen. In any of the embodiments in this paragraph, R⁴ is alkyl.

In another embodiment, R^{1a} is hydrogen and R^{1b} is alkenyl; R² is an amino acid side chain; R³ is hydrogen; D* is hydrogen; and n is two. In another embodiment, R^{1a} is hydrogen and R^{1b} is alkynyl; R² is an amino acid side chain; R³ is hydrogen; D* is hydrogen; and n is two. In another embodiment, R^{1a} is hydrogen and R^{1b} is aryl; R² is an amino acid side chain; R³ is hydrogen; D* is hydrogen; and n is two. In another embodiment, R^{1a} is hydrogen and R^{1b} is arylalkyl; R² is an amino acid side chain; R³ is hydrogen; D* is hydrogen; and n is two. In another embodiment, R^{1a} is hydrogen and R^{1b} is heteroaryl; R² is an amino acid side chain; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is hydrogen; and n is two. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is hydrogen; and n is two. In another embodiment, R^{1a} is hydrogen and R^{1b} is hydrogen; R² is hydrogen; R³ is alkyl; D* is hydrogen; and n is two. In another embodiment, R^{1a} is hydrogen and R^{1b} is alkyl; R² is hydrogen; R³ is alkyl; D* is hydrogen; and n is two. In another embodiment, R^{1a} is hydrogen and R^{1b} is alkoxy; R² is hydrogen; R³ is alkyl; D* is hydrogen; and n is two. In another embodiment, R^{1a} is hydrogen and R^{1b} is alkenyl; R² is hydrogen; R³ is alkyl; D* is hydrogen; and n is two. In another embodiment, R^{1a} is hydrogen and R^{1b} is alkynyl; R² is hydrogen; R³ is alkyl; D* is hydrogen; and n is two. In any of the embodiments in this paragraph, R⁴ is hydrogen. In any of the embodiments in this paragraph, R⁴ is alkyl.

In another embodiment, R^{1a} is hydrogen and R^{1b} is aryl; R² is hydrogen; R³ is alkyl; D* is hydrogen; and n is two. In another embodiment, R^{1a} is hydrogen and R^{1b} is arylalkyl; R² is hydrogen; R³ is alkyl; D* is hydrogen; and n is two. In another embodiment, R^{1a} is hydrogen and R^{1b} is heteroaryl; R² is hydrogen; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is hydrogen; and n is two. In another embodiment, R^{1a} is hydrogen and R^{1b} is hydrogen; R² is an amino acid side chain; R³ is alkyl; D* is hydrogen; and n is two. In another embodiment, R^{1a} is hydrogen and R^{1b} is alkyl; R² is amino acid side chain; R³ is alkyl; D* is hydrogen; and n is two. In another embodiment, R^{1a} is hydrogen and R^{1b} is alkoxy; R² is an amino acid side chain; R³ is alkyl; D* is hydrogen; and n is two. In another embodiment, R^{1a} is hydrogen and R^{1b} is alkenyl; R² is an amino acid side chain; R³ is alkyl; D* is hydrogen; and n is two. In another embodiment, R^{1a} is hydrogen and R^{1b} is alkynyl; R² is an amino acid side chain; R³ is alkyl; D* is hydrogen; and n is two. In another embodiment, R^{1a} is hydrogen and R^{1b} is aryl; R² is an amino acid side chain; R³ is alkyl; D* is hydrogen; and n is two. In any of the embodiments in this paragraph, R⁴ is hydrogen. In any of the embodiments in this paragraph, R⁴ is alkyl.

In another embodiment, R^{1a} is hydrogen and R^{1b} is arylalkyl; R² is an amino acid side chain; R³ is alkyl; D* is hydrogen; and n is two. In another embodiment, R^{1a} is hydrogen and R^{1b} is heteroaryl; R² is an amino acid side chain; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is hydrogen; and n is two. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is hydrogen; and n is two. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is hydrogen; and n is two. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is hydrogen; and n is two. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4-, 5-, or 6-membered heterocyclyl; D* is hydrogen; and n is two. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is hydrogen; and n is two. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is hydrogen; and n is two. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is hydrogen; and n is two. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is hydrogen; and n is two. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is hydrogen; and n is two. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is hydrogen; and n is two. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is hydrogen; and n is two. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is hydrogen; and n is two. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is hydrogen; and n is two. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is hydrogen; and n is two. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is hydrogen; and n is two. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is hydrogen; and n is two. In another embodiment, R^{1a} is hydrogen and R^{1b} is hydrogen; R² is hydrogen; R³ is hydrogen; D* is acyl; and n is two. In another embodiment, R^{1a} is hydrogen and R^{1b} is alkyl; R² is hydrogen; R³ is hydrogen; D* is acyl; and n is two. In another embodiment, R^{1a} is hydrogen and R^{1b} is alkoxy; R² is hydrogen; R³ is hydrogen; D* is acyl; and n is two. In another embodiment, R^{1a} is hydrogen and R^{1b} is alkenyl; R² is hydrogen; R³ is hydrogen; D* is acyl; and n is two. In another embodiment, R^{1a} is hydrogen and R^{1b} is alkynyl; R² is hydrogen; R³ is hydrogen; D* is acyl; and n is two. In another embodiment, R^{1a} is hydrogen and R^{1b} is aryl; R² is hydrogen; R³ is hydrogen; D* is acyl; and n is two. In another embodiment, R^{1a} is hydrogen and R^{1b} is arylalkyl; R² is hydrogen; R³ is hydrogen; D* is acyl; and n is two. In another embodiment, R^{1a} is hydrogen and R^{1b} is heteroaryl; R² is hydrogen; R³ is hydrogen; D* is acyl; and n is two. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is acyl; and n is two. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is acyl; and n is two. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is acyl; and n is two. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is acyl; and n is two. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is acyl; and n is two. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is acyl; and n is two. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is acyl; and n is two. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is acyl; and n is two. In another embodiment, R^{1a} is hydrogen and R^{1b} is hydrogen; R² is an amino acid side chain; R³ is hydrogen; D* is acyl; and n is two. In another embodiment, R^{1a} is hydrogen and R^{1b} is alkyl; R² is amino acid side chain; R³ is hydrogen; D* is acyl; and n is two. In another embodiment, R^{1a} is hydrogen and R^{1b} is alkoxy; R² is an amino acid side chain; R³ is hydrogen; D* is acyl; and n is two. In another embodiment, R^{1a} is hydrogen and R^{1b} is alkenyl; R² is an amino acid side chain; R³ is hydrogen; D* is acyl; and n is two. In another embodiment, R^{1a} is hydrogen and R^{1b} is alkynyl; R² is an amino acid side chain; R³ is hydrogen; D* is acyl; and n is two. In another embodiment, R^{1a} is hydrogen and R^{1b} is aryl; R² is an amino acid side chain; R³ is hydrogen; D* is acyl; and n is two. In another embodiment, R^{1a} is hydrogen and R^{1b} is arylalkyl; R² is an amino acid side chain; R³ is hydrogen; D* is acyl; and n is two. In another embodiment, R^{1a} is hydrogen and R^{1b} is heteroaryl; R² is an amino acid side chain; R³ is hydrogen; D* is acyl; and n is two. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is acyl; and n is two. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is acyl; and n is two. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is acyl; and n is two. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is acyl; and n is two. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is acyl; and n is two. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is acyl; and n is two. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is acyl; and n is two. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is acyl; and n is two. In any of the embodiments in this paragraph, R⁴ is hydrogen. In any of the embodiments in this paragraph, R⁴ is alkyl.

In another embodiment, R^{1a} is hydrogen and R^{1b} is hydrogen; R² is hydrogen; R³ is alkyl; D* is acyl; and n is two. In another embodiment, R^{1a} is hydrogen and R^{1b} is alkyl; R² is hydrogen; R³ is alkyl; D* is acyl; and n is two. In another embodiment, R^{1a} is hydrogen and R^{1b} is alkoxy; R² is hydrogen; R³ is alkyl; D* is acyl; and n is two. In another embodiment, R^{1a} is hydrogen and R^{1b} is alkenyl; R² is hydrogen; R³ is alkyl; D* is acyl; and n is two. In another embodiment, R^{1a} is hydrogen and R^{1b} is alkynyl; R² is hydrogen; R³ is alkyl; D* is acyl; and n is two. In another embodiment, R^{1a} is hydrogen and R^{1b} is aryl; R² is hydrogen; R³ is alkyl; D* is acyl; and n is two. In another embodiment, R^{1a} is hydrogen and R^{1b} is arylalkyl; R² is hydrogen; R³ is alkyl; D* is acyl; and n is two. In another embodiment, R^{1a} is hydrogen and R^{1b} is heteroaryl; R² is hydrogen; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is acyl; and n is two. In another embodiment, R^{1a} is hydrogen and R^{1b} is hydrogen; R² is an amino acid side chain; R³ is alkyl; D* is acyl; and n is two. In another embodiment, R^{1a} is hydrogen and R^{1b} is alkyl; R² is amino acid side chain; R³ is alkyl; D* is acyl; and n is two. In another embodiment, R^{1a} is hydrogen and R^{1b} is alkoxy; R² is an amino acid side chain; R³ is alkyl; D* is acyl; and n is two. In another embodiment, R^{1a} is hydrogen and R^{1b} is alkenyl; R² is an amino acid side chain; R³ is alkyl; D* is acyl; and n is two. In any of the embodiments in this paragraph, R⁴ is hydrogen. In any of the embodiments in this paragraph, R⁴ is alkyl.

In another embodiment, R^{1a} is hydrogen and R^{1b} is alkynyl; R² is an amino acid side chain; R³ is alkyl; D* is acyl; and n is two. In another embodiment, R^{1a} is hydrogen and R^{1b} is aryl; R² is an amino acid side chain; R³ is alkyl; D* is acyl; and n is two. In another embodiment, R^{1a} is hydrogen and R^{1b} is arylalkyl; R² is an amino acid side chain; R³ is alkyl; D* is acyl; and n is two. In another embodiment, R^{1a} is hydrogen and R^{1b} is heteroaryl; R² is an amino acid side chain; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is acyl; and n is two. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is acyl; and n is two. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is acyl; and n is two. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is acyl; and n is two. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4-, 5-, or 6-membered heterocyclyl; D* is acyl; and n is two. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is acyl; and n is two. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is acyl; and n is two. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is acyl; and n is two. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is acyl; and n is two. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is acyl; and n is two. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is acyl; and n is two. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is acyl; and n is two. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is acyl; and n is two. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is acyl; and n is two. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is acyl; and n is two. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is acyl; and n is two. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is acyl; and n is two. In another embodiment, R^{1a} is hydrogen and R^{1b} is hydrogen; R² is hydrogen; R³ is hydrogen; D* is a residue of a biologically active compound; and n is two. In another embodiment, R^{1a} is hydrogen and R^{1b} is alkyl; R² is hydrogen; R³ is hydrogen; D* is a residue of a biologically active compound; and n is two. In another embodiment, R^{1a} is hydrogen and R^{1b} is alkoxy; R² is hydrogen; R³ is hydrogen; D* is a residue of a biologically active compound; and n is two. In another embodiment, R^{1a} is hydrogen and R^{1b} is alkenyl; R² is hydrogen; R³ is hydrogen; D* is a residue of a biologically active compound; and n is two. In another embodiment, R^{1a} is hydrogen and R^{1b} is alkynyl; R² is hydrogen; R³ is hydrogen; D* is a residue of a biologically active compound; and n is two. In another embodiment, R^{1a} is hydrogen and R^{1b} is aryl; R² is hydrogen; R³ is hydrogen; D* is a residue of a biologically active compound; and n is two. In another embodiment, R^{1a} is hydrogen and R^{1b} is arylalkyl; R² is hydrogen; R³ is hydrogen; D* is a residue of a biologically active compound; and n is two. In another embodiment, R^{1a} is hydrogen and R^{1b} is heteroaryl; R² is hydrogen; R³ is hydrogen; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is a residue of a biologically active compound; and n is two. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is a residue of a biologically active compound; and n is two. In another embodiment, R^{1a} is hydrogen and R^{1b} is hydrogen; R² is an amino acid side chain; R³ is hydrogen; D* is a residue of a biologically active compound; and n is two. In another embodiment, R^{1a} is hydrogen and R^{1b} is alkyl; R² is amino acid side chain; R³ is hydrogen; D* is a residue of a biologically active compound; and n is two. In another embodiment, R^{1a} is hydrogen and R^{1b} is alkoxy; R² is an amino acid side chain; R³ is hydrogen; D* is a residue of a biologically active compound; and n is two. In another embodiment, R^{1a} is hydrogen and R^{1b} is alkenyl; R² is an amino acid side chain; R³ is hydrogen; D* is a residue of a biologically active compound; and n is two. In another embodiment, R^{1a} is hydrogen and R^{1b} is alkynyl; R² is an amino acid side chain; R³ is hydrogen; D* is a residue of a biologically active compound; and n is two. In another embodiment, R^{1a} is hydrogen and R^{1b} is aryl; R² is an amino acid side chain; R³ is hydrogen; D* is a residue of a biologically active compound; and n is two. In another embodiment, R^{1a} is hydrogen and R^{1b} is arylalkyl; R² is an amino acid side chain; R³ is hydrogen; D* is a residue of a biologically active compound; and n is two. In another embodiment, R^{1a} is hydrogen and R^{1b} is heteroaryl; R² is an amino acid side chain; R³ is hydrogen; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is a residue of a biologically active compound; and n is two. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is a residue of a biologically active compound; and n is two. In another embodiment, R^{1a} is hydrogen and R^{1b} is hydrogen; R² is hydrogen; R³ is alkyl; D* is a residue of a biologically active compound; and n is two. In another embodiment, R^{1a} is hydrogen and R^{1b} is alkyl; R² is hydrogen; R³ is alkyl; D* is a residue of a biologically active compound; and n is two. In another embodiment, R^{1a} is hydrogen and R^{1b} is alkoxy; R² is hydrogen; R³ is alkyl; D* is a residue of a biologically active compound; and n is two. In another embodiment, R^{1a} is hydrogen and R^{1b} is alkenyl; R² is hydrogen; R³ is alkyl; D* is a residue of a biologically active compound; and n is two. In another embodiment, R^{1a} is hydrogen and R^{1b} is alkynyl; R² is hydrogen; R³ is alkyl; D* is a residue of a biologically active compound; and n is two. In another embodiment, R^{1a} is hydrogen and R^{1b} is aryl; R² is hydrogen; R³ is alkyl; D* is a residue of a biologically active compound; and n is two. In another embodiment, R^{1a} is hydrogen and R^{1b} is arylalkyl; R² is hydrogen; R³ is alkyl; D* is a residue of a biologically active compound; and n is two. In another embodiment, R^{1a} is hydrogen and R^{1b} is heteroaryl; R² is hydrogen; R³ is alkyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is a residue of a biologically active compound; and n is two. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is a residue of a biologically active compound; and n is two. In another embodiment, R^{1a} is hydrogen and R^{1b} is hydrogen; R² is an amino acid side chain; R³ is alkyl; D* is a residue of a biologically active compound; and n is two. In another embodiment, R^{1a} is hydrogen and R^{1b} is alkyl; R² is amino acid side chain; R³ is alkyl; D* is a residue of a biologically active compound; and n is two. In another embodiment, R^{1a} is hydrogen and R^{1b} is alkoxy; R² is an amino acid side chain; R³ is alkyl; D* is a residue of a biologically active compound; and n is two. In another embodiment, R^{1a} is hydrogen and R^{1b} is alkenyl; R² is an amino acid side chain; R³ is alkyl; D* is a residue of a biologically active compound; and n is two. In another embodiment, R^{1a} is hydrogen and R^{1b} is alkynyl; R² is an amino acid side chain; R³ is alkyl; D* is a residue of a biologically active compound; and n is two. In another embodiment, R^{1a} is hydrogen and R^{1b} is aryl; R² is an amino acid side chain; R³ is alkyl; D* is a residue of a biologically active compound; and n is two. In another embodiment, R^{1a} is hydrogen and R^{1b} is arylalkyl; R² is an amino acid side chain; R³ is alkyl; D* is a residue of a biologically active compound; and n is two. In another embodiment, R^{1a} is hydrogen and R^{1b} is heteroaryl; R² is an amino acid side chain; R³ is alkyl; D* is a residue of a biologically active compound; and n is two. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is a residue of a biologically active compound; and n is two. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4-, 5-, or 6-membered heterocyclyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is a residue of a biologically active compound; and n is two. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is a residue of a biologically active compound; and n is two. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is a residue of a biologically active compound; and n is two. In any of the embodiments in this paragraph, R⁴ is hydrogen. In any of the embodiments in this paragraph, R⁴ is alkyl.

In another embodiment, R^{1a} is hydrogen and R^{1b} is hydrogen; R² is hydrogen; R³ is hydrogen; D* is hydrogen; and n is three. In another embodiment, R^{1a} is hydrogen and R^{1b} is alkyl; R² is hydrogen; R³ is hydrogen; D* is hydrogen; and n is three. In another embodiment, R^{1a} is hydrogen and R^{1b} is alkoxy; R² is hydrogen; R³ is hydrogen; D* is hydrogen; and n is three. In another embodiment, R^{1a} is hydrogen and R^{1b} is alkenyl; R² is hydrogen; R³ is hydrogen; D* is hydrogen; and n is three. In another embodiment, R^{1a} is hydrogen and R^{1b} is alkynyl; R² is hydrogen; R³ is hydrogen; D* is hydrogen; and n is three. In another embodiment, R^{1a} is hydrogen and R^{1b} is aryl; R² is hydrogen; R³ is hydrogen; D* is hydrogen; and n is three. In another embodiment, R^{1a} is hydrogen and R^{1b} is arylalkyl; R² is hydrogen; R³ is hydrogen; D* is hydrogen; and n is three. In another embodiment, R^{1a} is hydrogen and R^{1b} is heteroaryl; R² is hydrogen; R³ is hydrogen; D* is hydrogen; and n is three. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is hydrogen; and n is three. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is hydrogen; and n is three. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is hydrogen; and n is three. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is hydrogen; and n is three. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is hydrogen; and n is three. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is hydrogen; and n is three. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is hydrogen; and n is three. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is hydrogen; and n is three. In another embodiment, R^{1a} is hydrogen and R^{1b} is hydrogen; R² is an amino acid side chain; R³ is hydrogen; D* is hydrogen; and n is three. In another embodiment, R^{1a} is hydrogen and R^{1b} is alkyl; R² is amino acid side chain; R³ is hydrogen; D* is hydrogen; and n is three. In another embodiment, R^{1a} is hydrogen and R^{1b} is alkoxy; R² is an amino acid side chain; R³ is hydrogen; D* is hydrogen; and n is three. In another embodiment, R^{1a} is hydrogen and R^{1b} is alkenyl; R² is an amino acid side chain; R³ is hydrogen; D* is hydrogen; and n is three. In another embodiment, R^{1a} is hydrogen and R^{1b} is alkynyl; R² is an amino acid side chain; R³ is hydrogen; D* is hydrogen; and n is three. In another embodiment, R^{1a} is hydrogen and R^{1b} is aryl; R² is an amino acid side chain; R³ is hydrogen; D* is hydrogen; and n is three. In another embodiment, R^{1a} is hydrogen and R^{1b} is arylalkyl; R² is an amino acid side chain; R³ is hydrogen; D* is hydrogen; and n is three. In another embodiment, R^{1a} is hydrogen and R^{1b} is heteroaryl; R² is an amino acid side chain; R³ is hydrogen; D* is hydrogen; and n is three. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is hydrogen; and n is three. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is hydrogen; and n is three. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is hydrogen; and n is three. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is hydrogen; and n is three. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is hydrogen; and n is three. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is hydrogen; and n is three. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is hydrogen; and n is three. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is hydrogen; and n is three. In another embodiment, R^{1a} is hydrogen and R^{1b} is hydrogen; R² is hydrogen; R³ is alkyl; D* is hydrogen; and n is three. In another embodiment, R^{1a} is hydrogen and R^{1b} is alkyl; R² is hydrogen; R³ is alkyl; D* is hydrogen; and n is three. In another embodiment, R^{1a} is hydrogen and R^{1b} is alkoxy; R² is hydrogen; R³ is alkyl; D* is hydrogen; and n is three. In another embodiment, R^{1a} is hydrogen and R^{1b} is alkenyl; R² is hydrogen; R³ is alkyl; D* is hydrogen; and n is three. In another embodiment, R^{1a} is hydrogen and R^{1b} is alkynyl; R² is hydrogen; R³ is alkyl; D* is hydrogen; and n is three. In another embodiment, R^{1a} is hydrogen and R^{1b} is aryl; R² is hydrogen; R³ is alkyl; D* is hydrogen; and n is three. In another embodiment, R^{1a} is hydrogen and R^{1b} is arylalkyl; R² is hydrogen; R³ is alkyl; D* is hydrogen; and n is three. In another embodiment, R^{1a} is hydrogen and R^{1b} is heteroaryl; R² is hydrogen; R³ is alkyl; D* is hydrogen; and n is three. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is hydrogen; and n is three. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is hydrogen; and n is three. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is hydrogen; and n is three. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is hydrogen; and n is three. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is hydrogen; and n is three. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is hydrogen; and n is three. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is hydrogen; and n is three. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is hydrogen; and n is three. In another embodiment, R^{1a} is hydrogen and R^{1b} is hydrogen; R² is an amino acid side chain; R³ is alkyl; D* is hydrogen; and n is three. In another embodiment, R^{1a} is hydrogen and R^{1b} is alkyl; R² is amino acid side chain; R³ is alkyl; D* is hydrogen; and n is three. In another embodiment, R^{1a} is hydrogen and R^{1b} is alkoxy; R² is an amino acid side chain; R³ is alkyl; D* is hydrogen; and n is three. In another embodiment, R^{1a} is hydrogen and R^{1b} is alkenyl; R² is an amino acid side chain; R³ is alkyl; D* is hydrogen; and n is three. In another embodiment, R^{1a} is hydrogen and R^{1b} is alkynyl; R² is an amino acid side chain; R³ is alkyl; D* is hydrogen; and n is three. In another embodiment, R^{1a} is hydrogen and R^{1b} is aryl; R² is an amino acid side chain; R³ is alkyl; D* is hydrogen; and n is three. In another embodiment, R^{1a} is hydrogen and R^{1b} is arylalkyl; R² is an amino acid side chain; R³ is alkyl; D* is hydrogen; and n is three. In another embodiment, R^{1a} is hydrogen and R^{1b} is heteroaryl; R² is an amino acid side chain; R³ is alkyl; D* is hydrogen; and n is three. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is hydrogen; and n is three. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is hydrogen; and n is three. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is hydrogen; and n is three. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is hydrogen; and n is three. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is hydrogen; and n is three. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is hydrogen; and n is three. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is hydrogen; and n is three. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is hydrogen; and n is three. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is hydrogen; and n is three. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is hydrogen; and n is three. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is hydrogen; and n is three. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4-, 5-, or 6-membered heterocyclyl; D* is hydrogen; and n is three. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is hydrogen; and n is three. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is hydrogen; and n is three. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is hydrogen; and n is three. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is hydrogen; and n is three. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is hydrogen; and n is three. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is hydrogen; and n is three. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is hydrogen; and n is three. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is hydrogen; and n is three. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is hydrogen; and n is three. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is hydrogen; and n is three. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is hydrogen; and n is three. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is hydrogen; and n is three. In another embodiment, R^{1a} is hydrogen and R^{1b} is hydrogen; R² is hydrogen; R³ is hydrogen; D* is acyl; and n is three. In another embodiment, R^{1a} is hydrogen and R^{1b} is alkyl; R² is hydrogen; R³ is hydrogen; D* is acyl; and n is three. In another embodiment, R^{1a} is hydrogen and R^{1b} is alkoxy; R² is hydrogen; R³ is hydrogen; D* is acyl; and n is three. In another embodiment, R^{1a} is hydrogen and R^{1b} is alkenyl; R² is hydrogen; R³ is hydrogen; D* is acyl; and n is three. In another embodiment, R^{1a} is hydrogen and R^{1b} is alkynyl; R² is hydrogen; R³ is hydrogen; D* is acyl; and n is three. In another embodiment, R^{1a} is hydrogen and R^{1b} is aryl; R² is hydrogen; R³ is hydrogen; D* is acyl; and n is three. In another embodiment, R^{1a} is hydrogen and R^{1b} is arylalkyl; R² is hydrogen; R³ is hydrogen; D* is acyl; and n is three. In another embodiment, R^{1a} is hydrogen and R^{1b} is heteroaryl; R² is hydrogen; R³ is hydrogen; D* is acyl; and n is three. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is acyl; and n is three. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is acyl; and n is three. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is acyl; and n is three. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is acyl; and n is three. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is acyl; and n is three. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is acyl; and n is three. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is acyl; and n is three. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is acyl; and n is three. In another embodiment, R^{1a} is hydrogen and R^{1b} is hydrogen; R² is an amino acid side chain; R³ is hydrogen; D* is acyl; and n is three. In another embodiment, R^{1a} is hydrogen and R^{1b} is alkyl; R² is amino acid side chain; R³ is hydrogen; D* is acyl; and n is three. In another embodiment, R^{1a} is hydrogen and R^{1b} is alkoxy; R² is an amino acid side chain; R³ is hydrogen; D* is acyl; and n is three. In another embodiment, R^{1a} is hydrogen and R^{1b} is alkenyl; R² is an amino acid side chain; R³ is hydrogen; D* is acyl; and n is three. In another embodiment, R^{1a} is hydrogen and R^{1b} is alkynyl; R² is an amino acid side chain; R³ is hydrogen; D* is acyl; and n is three. In another embodiment, R^{1a} is hydrogen and R^{1b} is aryl; R² is an amino acid side chain; R³ is hydrogen; D* is acyl; and n is three. In another embodiment, R^{1a} is hydrogen and R^{1b} is arylalkyl; R² is an amino acid side chain; R³ is hydrogen; D* is acyl; and n is three. In another embodiment, R^{1a} is hydrogen and R^{1b} is heteroaryl; R² is an amino acid side chain; R³ is hydrogen; D* is acyl; and n is three. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is acyl; and n is three. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is acyl; and n is three. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is acyl; and n is three. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is acyl; and n is three. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is acyl; and n is three. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is acyl; and n is three. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is acyl; and n is three. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is acyl; and n is three. In another embodiment, R^{1a} is hydrogen and R^{1b} is hydrogen; R² is hydrogen; R³ is alkyl; D* is acyl; and n is three. In another embodiment, R^{1a} is hydrogen and R^{1b} is alkyl; R² is hydrogen; R³ is alkyl; D* is acyl; and n is three. In another embodiment, R^{1a} is hydrogen and R^{1b} is alkoxy; R² is hydrogen; R³ is alkyl; D* is acyl; and n is three. In another embodiment, R^{1a} is hydrogen and R^{1b} is alkenyl; R² is hydrogen; R³ is alkyl; D* is acyl; and n is three. In another embodiment, R^{1a} is hydrogen and R^{1b} is alkynyl; R² is hydrogen; R³ is alkyl; D* is acyl; and n is three. In another embodiment, R^{1a} is hydrogen and R^{1b} is aryl; R² is hydrogen; R³ is alkyl; D* is acyl; and n is three. In another embodiment, R^{1a} is hydrogen and R^{1b} is arylalkyl; R² is hydrogen; R³ is alkyl; D* is acyl; and n is three. In another embodiment, R^{1a} is hydrogen and R^{1b} is heteroaryl; R² is hydrogen; R³ is alkyl; D* is acyl; and n is three. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is acyl; and n is three. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is acyl; and n is three. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is acyl; and n is three. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is acyl; and n is three. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is acyl; and n is three. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is acyl; and n is three. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is acyl; and n is three. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is acyl; and n is three. In another embodiment, R^{1a} is hydrogen and R^{1b} is hydrogen; R² is an amino acid side chain; R³ is alkyl; D* is acyl; and n is three. In another embodiment, R^{1a} is hydrogen and R^{1b} is alkyl; R² is amino acid side chain; R³ is alkyl; D* is acyl; and n is three. In another embodiment, R^{1a} is hydrogen and R^{1b} is alkoxy; R² is an amino acid side chain; R³ is alkyl; D* is acyl; and n is three. In another embodiment, R^{1a} is hydrogen and R^{1b} is alkenyl; R² is an amino acid side chain; R³ is alkyl; D* is acyl; and n is three. In another embodiment, R^{1a} is hydrogen and R^{1b} is alkynyl; R² is an amino acid side chain; R³ is alkyl; D* is acyl; and n is three. In another embodiment, R^{1a} is hydrogen and R^{1b} is aryl; R² is an amino acid side chain; R³ is alkyl; D* is acyl; and n is three. In another embodiment, R^{1a} is hydrogen and R^{1b} is arylalkyl; R² is an amino acid side chain; R³ is alkyl; D* is acyl; and n is three. In another embodiment, R^{1a} is hydrogen and R^{1b} is heteroaryl; R² is an amino acid side chain; R³ is alkyl; D* is acyl; and n is three. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is acyl; and n is three. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is acyl; and n is three. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is acyl; and n is three. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is acyl; and n is three. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is acyl; and n is three. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is acyl; and n is three. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is acyl; and n is three. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is acyl; and n is three. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is acyl; and n is three. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is acyl; and n is three. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is acyl; and n is three. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4-, 5-, or 6-membered heterocyclyl; D* is acyl; and n is three. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is acyl; and n is three. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is acyl; and n is three. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is acyl; and n is three. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is acyl; and n is three. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is acyl; and n is three. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is acyl; and n is three. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is acyl; and n is three. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is acyl; and n is three. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is acyl; and n is three. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is acyl; and n is three. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is acyl; and n is three. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is acyl; and n is three. In another embodiment, R^{1a} is hydrogen and R^{1b} is hydrogen; R² is hydrogen; R³ is hydrogen; D* is a residue of a biologically active compound; and n is three. In another embodiment, R^{1a} is hydrogen and R^{1b} is alkyl; R² is hydrogen; R³ is hydrogen; D* is a residue of a biologically active compound; and n is three. In another embodiment, R^{1a} is hydrogen and R^{1b} is alkoxy; R² is hydrogen; R³ is hydrogen; D* is a residue of a biologically active compound; and n is three. In another embodiment, R^{1a} is hydrogen and R^{1b} is alkenyl; R² is hydrogen; R³ is hydrogen; D* is a residue of a biologically active compound; and n is three. In another embodiment, R^{1a} is hydrogen and R^{1b} is alkynyl; R² is hydrogen; R³ is hydrogen; D* is a residue of a biologically active compound; and n is three. In any of the embodiments in this paragraph, R⁴ is hydrogen. In any of the embodiments in this paragraph, R⁴ is alkyl.

In another embodiment, R^{1a} is hydrogen and R^{1b} is aryl; R² is hydrogen; R³ is hydrogen; D* is a residue of a biologically active compound; and n is three. In another embodiment, R^{1a} is hydrogen and R^{1b} is arylalkyl; R² is hydrogen; R³ is hydrogen; D* is a residue of a biologically active compound; and n is three. In another embodiment, R^{1a} is hydrogen and R^{1b} is heteroaryl; R² is hydrogen; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is a residue of a biologically active compound; and n is three. In another embodiment, R^{1a} is hydrogen and R^{1b} is hydrogen; R² is an amino acid side chain; R³ is hydrogen; D* is a residue of a biologically active compound; and n is three. In another embodiment, R^{1a} is hydrogen and R^{1b} is alkyl; R² is amino acid side chain; R³ is hydrogen; D* is a residue of a biologically active compound; and n is three. In another embodiment, R^{1a} is hydrogen and R^{1b} is alkoxy; R² is an amino acid side chain; R³ is hydrogen; D* is a residue of a biologically active compound; and n is three. In any of the embodiments in this paragraph, R⁴ is hydrogen. In any of the embodiments in this paragraph, R⁴ is alkyl.

In another embodiment, R^{1a} is hydrogen and R^{1b} is alkenyl; R² is an amino acid side chain; R³ is hydrogen; D* is a residue of a biologically active compound; and n is three. In another embodiment, R^{1a} is hydrogen and R^{1b} is alkynyl; R² is an amino acid side chain; R³ is hydrogen; D* is a residue of a biologically active compound; and n is three. In another embodiment, R^{1a} is hydrogen and R^{1b} is aryl; R² is an amino acid side chain; R³ is hydrogen; D* is a residue of a biologically active compound; and n is three. In another embodiment, R^{1a} is hydrogen and R^{1b} is arylalkyl; R² is an amino acid side chain; R³ is hydrogen; D* is a residue of a biologically active compound; and n is three. In another embodiment, R^{1a} is hydrogen and R^{1b} is heteroaryl; R² is an amino acid side chain; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is a residue of a biologically active compound; and n is three. In any of the embodiments in this paragraph, R⁴ is hydrogen. In any of the embodiments in this paragraph, R⁴ is alkyl.

In another embodiment, R^{1a} is hydrogen and R^{1b} is hydrogen; R² is hydrogen; R³ is alkyl; D* is a residue of a biologically active compound; and n is three. In another embodiment, R^{1a} is hydrogen and R^{1b} is alkyl; R² is hydrogen; R³ is alkyl; D* is a residue of a biologically active compound; and n is three. In another embodiment, R^{1a} is hydrogen and R^{1b} is alkoxy; R² is hydrogen; R³ is alkyl; D* is a residue of a biologically active compound; and n is three. In another embodiment, R^{1a} is hydrogen and R^{1b} is alkenyl; R² is hydrogen; R³ is alkyl; D* is a residue of a biologically active compound; and n is three. In another embodiment, R^{1a} is hydrogen and R^{1b} is alkynyl; R² is hydrogen; R³ is alkyl; D* is a residue of a biologically active compound; and n is three. In another embodiment, R^{1a} is hydrogen and R^{1b} is aryl; R² is hydrogen; R³ is alkyl; D* is a residue of a biologically active compound; and n is three. In another embodiment, R^{1a} is hydrogen and R^{1b} is arylalkyl; R² is hydrogen; R³ is alkyl; D* is a residue of a biologically active compound; and n is three. In another embodiment, R^{1a} is hydrogen and R^{1b} is heteroaryl; R² is hydrogen; R³ is alkyl; D* is a residue of a biologically active compound; and n is three. In another embodiment, Ria is alkylene, wherein the alkylene is further bonded to R³ to form a 4-, 5-, or 6-membered heterocyclyl; and R^{1b} is hydrogen; R² is hydrogen; R³ is alkyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is a residue of a biologically active compound; and n is three. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is a residue of a biologically active compound; and n is three. In another embodiment, R^{1a} is hydrogen and R^{1b} is hydrogen; R² is an amino acid side chain; R³ is alkyl; D* is a residue of a biologically active compound; and n is three. In another embodiment, R^{1a} is hydrogen and R^{1b} is alkyl; R² is amino acid side chain; R³ is alkyl; D* is a residue of a biologically active compound; and n is three. In another embodiment, R^{1a} is hydrogen and R^{1b} is alkoxy; R² is an amino acid side chain; R³ is alkyl; D* is a residue of a biologically active compound; and n is three. In another embodiment, R^{1a} is hydrogen and R^{1b} is alkenyl; R² is an amino acid side chain; R³ is alkyl; D* is a residue of a biologically active compound; and n is three. In another embodiment, R^{1a} is hydrogen and R^{1b} is alkynyl; R² is an amino acid side chain; R³ is alkyl; D* is a residue of a biologically active compound; and n is three. In another embodiment, R^{1a} is hydrogen and R^{1b} is aryl; R² is an amino acid side chain; R³ is alkyl; D* is a residue of a biologically active compound; and n is three. In another embodiment, R^{1a} is hydrogen and R^{1b} is arylalkyl; R² is an amino acid side chain; R³ is alkyl; D* is a residue of a biologically active compound; and n is three. In another embodiment, R^{1a} is hydrogen and R^{1b} is heteroaryl; R² is an amino acid side chain; R³ is alkyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is a residue of a biologically active compound; and n is three. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4-, 5-, or 6-membered heterocyclyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is a residue of a biologically active compound; and n is three. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is a residue of a biologically active compound; and n is three. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is a residue of a biologically active compound; and n is three. In any of the embodiments in this paragraph, R⁴ is hydrogen. In any of the embodiments in this paragraph, R⁴ is alkyl.

In another embodiment, R^{1a} is hydrogen and R^{1b} is hydrogen; R² is hydrogen; R³ is hydrogen; D* is hydrogen; and n is four. In another embodiment, R^{1a} is hydrogen and R^{1b} is alkyl; R² is hydrogen; R³ is hydrogen; D* is hydrogen; and n is four. In another embodiment, R^{1a} is hydrogen and R^{1b} is alkoxy; R² is hydrogen; R³ is hydrogen; D* is hydrogen; and n is four. In another embodiment, R^{1a} is hydrogen and R^{1b} is alkenyl; R² is hydrogen; R³ is hydrogen; D* is hydrogen; and n is four. In another embodiment, R^{1a} is hydrogen and R^{1b} is alkynyl; R² is hydrogen; R³ is hydrogen; D* is hydrogen; and n is four. In another embodiment, R^{1a} is hydrogen and R^{1b} is aryl; R² is hydrogen; R³ is hydrogen; D* is hydrogen; and n is four. In another embodiment, R^{1a} is hydrogen and R^{1b} is arylalkyl; R² is hydrogen; R³ is hydrogen; D* is hydrogen; and n is four. In another embodiment, R^{1a} is hydrogen and R^{1b} is heteroaryl; R² is hydrogen; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is hydrogen; and n is four. In another embodiment, R^{1a} is hydrogen and R^{1b} is hydrogen; R² is an amino acid side chain; R³ is hydrogen; D* is hydrogen; and n is four. In another embodiment, R^{1a} is hydrogen and R^{1b} is alkyl; R² is amino acid side chain; R³ is hydrogen; D* is hydrogen; and n is four. In another embodiment, R^{1a} is hydrogen and R^{1b} is alkoxy; R² is an amino acid side chain; R³ is hydrogen; D* is hydrogen; and n is four. In another embodiment, R^{1a} is hydrogen and R^{1b} is alkenyl; R² is an amino acid side chain; R³ is hydrogen; D* is hydrogen; and n is four. In another embodiment, R^{1a} is hydrogen and R^{1b} is alkynyl; R² is an amino acid side chain; R³ is hydrogen; D* is hydrogen; and n is four. In any of the embodiments in this paragraph, R⁴ is hydrogen. In any of the embodiments in this paragraph, R⁴ is alkyl.

In another embodiment, R^{1a} is hydrogen and R^{1b} is aryl; R² is an amino acid side chain; R³ is hydrogen; D* is hydrogen; In another embodiment, R^{1a} is hydrogen and R^{1b} is arylalkyl; R² is an amino acid side chain; R³ is hydrogen; D* is hydrogen; and n is four. In another embodiment, R^{1a} is hydrogen and R^{1b} is heteroaryl; R² is an amino acid side chain; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is hydrogen; and n is four. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is hydrogen; and n is four. In another embodiment, R^{1a} is hydrogen and R^{1b} is hydrogen; R² is hydrogen; R³ is alkyl; D* is hydrogen; and n is four. In another embodiment, R^{1a} is hydrogen and R^{1b} is alkyl; R² is hydrogen; R³ is alkyl; D* is hydrogen; and n is four. In another embodiment, R^{1a} is hydrogen and R^{1b} is alkoxy; R² is hydrogen; R³ is alkyl; D* is hydrogen; and n is four. In another embodiment, R^{1a} is hydrogen and R^{1b} is alkenyl; R² is hydrogen; R³ is alkyl; D* is hydrogen; and n is four. In another embodiment, R^{1a} is hydrogen and R^{1b} is alkynyl; R² is hydrogen; R³ is alkyl; D* is hydrogen; and n is four. In another embodiment, R^{1a} is hydrogen and R^{1b} is aryl; R² is hydrogen; R³ is alkyl; D* is hydrogen; and n is four. In any of the embodiments in this paragraph, R⁴ is hydrogen. In any of the embodiments in this paragraph, R⁴ is alkyl.

In another embodiment, R^{1a} is hydrogen and R^{1b} is arylalkyl; R² is hydrogen; R³ is alkyl; D* is hydrogen; and n is four. In another embodiment, R^{1a} is hydrogen and R^{1b} is heteroaryl; R² is hydrogen; R³ is alkyl; D* is hydrogen; and n is four. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is hydrogen; and n is four. In another embodiment, Ria is alkylene, wherein the alkylene is further bonded to R³ to form a 4-, 5-, or 6-membered heterocyclyl; and R^{1b} is alkyl; R² is hydrogen; R³ is alkyl; D* is hydrogen; and n is four. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is hydrogen; and n is four. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is hydrogen; and n is four. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is hydrogen; and n is four. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is hydrogen; and n is four. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is hydrogen; and n is four. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is hydrogen; and n is four. In another embodiment, R^{1a} is hydrogen and R^{1b} is hydrogen; R² is an amino acid side chain; R³ is alkyl; D* is hydrogen; and n is four. In another embodiment, R^{1a} is hydrogen and R^{1b} is alkyl; R² is amino acid side chain; R³ is alkyl; D* is hydrogen; and n is four. In another embodiment, R^{1a} is hydrogen and R^{1b} is alkoxy; R² is an amino acid side chain; R³ is alkyl; D* is hydrogen; and n is four. In another embodiment, R^{1a} is hydrogen and R^{1b} is alkenyl; R² is an amino acid side chain; R³ is alkyl; D* is hydrogen; and n is four. In another embodiment, R^{1a} is hydrogen and R^{1b} is alkynyl; R² is an amino acid side chain; R³ is alkyl; D* is hydrogen; and n is four. In another embodiment, R^{1a} is hydrogen and R^{1b} is aryl; R² is an amino acid side chain; R³ is alkyl; D* is hydrogen; and n is four. In another embodiment, R^{1a} is hydrogen and R^{1b} is arylalkyl; R² is an amino acid side chain; R³ is alkyl; D* is hydrogen; and n is four. In another embodiment, R^{1a} is hydrogen and R^{1b} is heteroaryl; R² is an amino acid side chain; R³ is alkyl; D* is hydrogen; and n is four. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is hydrogen; and n is four. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is hydrogen; and n is four. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is hydrogen; and n is four. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is hydrogen; and n is four. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is hydrogen; and n is four. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is hydrogen; and n is four. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is hydrogen; and n is four. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is hydrogen; and n is four. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is hydrogen; and n is four. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is hydrogen; and n is four. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is hydrogen; and n is four. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4-, 5-, or 6-membered heterocyclyl; D* is hydrogen; and n is four. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is hydrogen; and n is four. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is hydrogen; and n is four. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is hydrogen; and n is four. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is hydrogen; and n is four. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is hydrogen; and n is four. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is hydrogen; and n is four. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is hydrogen; and n is four. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is hydrogen; and n is four. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is hydrogen; and n is four. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is hydrogen; and n is four. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is hydrogen; and n is four. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is hydrogen; and n is four. In any of the embodiments in this paragraph, R⁴ is hydrogen. In any of the embodiments in this paragraph, R⁴ is alkyl.

In another embodiment, R^{1a} is hydrogen and R^{1b} is hydrogen; R² is hydrogen; R³ is hydrogen; D* is acyl; and n is four. In another embodiment, R^{1a} is hydrogen and R^{1b} is alkyl; R² is hydrogen; R³ is hydrogen; D* is acyl; and n is four. In another embodiment, R^{1a} is hydrogen and R^{1b} is alkoxy; R² is hydrogen; R³ is hydrogen; D* is acyl; and n is four. In another embodiment, R^{1a} is hydrogen and R^{1b} is alkenyl; R² is hydrogen; R³ is hydrogen; D* is acyl; and n is four. In another embodiment, R^{1a} is hydrogen and R^{1b} is alkynyl; R² is hydrogen; R³ is hydrogen; D* is acyl; and n is four. In another embodiment, R^{1a} is hydrogen and R^{1b} is aryl; R² is hydrogen; R³ is hydrogen; D* is acyl; and n is four. In another embodiment, R^{1a} is hydrogen and R^{1b} is arylalkyl; R² is hydrogen; R³ is hydrogen; D* is acyl; and n is four. In another embodiment, R^{1a} is hydrogen and R^{1b} is heteroaryl; R² is hydrogen; R³ is hydrogen; D* is acyl; and n is four. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is acyl; and n is four. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is acyl; and n is four. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is acyl; and n is four. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is acyl; and n is four. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is acyl; and n is four. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is acyl; and n is four. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is acyl; and n is four. In another embodiment, Ria is alkylene, wherein the alkylene is further bonded to R³ to form a 4-, 5-, or 6-membered heterocyclyl; and R^{1b} is heteroaryl; R² is hydrogen; R³ is hydrogen; D* is acyl; and n is four. In another embodiment, R^{1a} is hydrogen and R^{1b} is hydrogen; R² is an amino acid side chain; R³ is hydrogen; D* is acyl; and n is four. In another embodiment, R^{1a} is hydrogen and R^{1b} is alkyl; R² is amino acid side chain; R³ is hydrogen; D* is acyl; and n is four. In any of the embodiments in this paragraph, R⁴ is hydrogen. In any of the embodiments in this paragraph, R⁴ is alkyl.

In another embodiment, R^{1a} is hydrogen and R^{1b} is alkoxy; R² is an amino acid side chain; R³ is hydrogen; D* is acyl; and n is four. In another embodiment, R^{1a} is hydrogen and R^{1b} is alkenyl; R² is an amino acid side chain; R³ is hydrogen; D* is acyl; and n is four. In another embodiment, R^{1a} is hydrogen and R^{1b} is alkynyl; R² is an amino acid side chain; R³ is hydrogen; D* is acyl; and n is four. In another embodiment, R^{1a} is hydrogen and R^{1b} is aryl; R² is an amino acid side chain; R³ is hydrogen; D* is acyl; and n is four. In another embodiment, R^{1a} is hydrogen and R^{1b} is arylalkyl; R² is an amino acid side chain; R³ is hydrogen; D* is acyl; and n is four. In another embodiment, R^{1a} is hydrogen and R^{1b} is heteroaryl; R² is an amino acid side chain; R³ is hydrogen; D* is acyl; and n is four. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is acyl; and n is four. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is acyl; and n is four. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is acyl; and n is four. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is acyl; and n is four. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is acyl; and n is four. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is acyl; and n is four. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is acyl; and n is four. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is acyl; and n is four. In another embodiment, R^{1a} is hydrogen and R^{1b} is hydrogen; R² is hydrogen; R³ is alkyl; D* is acyl; and n is four. In another embodiment, R^{1a} is hydrogen and R^{1b} is alkyl; R² is hydrogen; R³ is alkyl; D* is acyl; and n is four. In another embodiment, R^{1a} is hydrogen and R^{1b} is alkoxy; R² is hydrogen; R³ is alkyl; D* is acyl; and n is four. In any of the embodiments in this paragraph, R⁴ is hydrogen. In any of the embodiments in this paragraph, R⁴ is alkyl.

In another embodiment, R^{1a} is hydrogen and R^{1b} is alkenyl; R² is hydrogen; R³ is alkyl; D* is acyl; and n is four. In another embodiment, R^{1a} is hydrogen and R^{1b} is alkynyl; R² is hydrogen; R³ is alkyl; D* is acyl; and n is four. In another embodiment, R^{1a} is hydrogen and R^{1b} is aryl; R² is hydrogen; R³ is alkyl; D* is acyl; and n is four. In another embodiment, R^{1a} is hydrogen and R^{1b} is arylalkyl; R² is hydrogen; R³ is alkyl; D* is acyl; and n is four. In another embodiment, R^{1a} is hydrogen and R^{1b} is heteroaryl; R² is hydrogen; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is acyl; and n is four. In another embodiment, R^{1a} is hydrogen and R^{1b} is hydrogen; R² is an amino acid side chain; R³ is alkyl; D* is acyl; and n is four. In another embodiment, R^{1a} is hydrogen and R^{1b} is alkyl; R² is amino acid side chain; R³ is alkyl; D* is acyl; and n is four. In another embodiment, R^{1a} is hydrogen and R^{1b} is alkoxy; R² is an amino acid side chain; R³ is alkyl; D* is acyl; and n is four. In another embodiment, R^{1a} is hydrogen and R^{1b} is alkenyl; R² is an amino acid side chain; R³ is alkyl; D* is acyl; and n is four. In another embodiment, R^{1a} is hydrogen and R^{1b} is alkynyl; R² is an amino acid side chain; R³ is alkyl; D* is acyl; and n is four. In another embodiment, R^{1a} is hydrogen and R^{1b} is aryl; R² is an amino acid side chain; R³ is alkyl; D* is acyl; and n is four. In another embodiment, R^{1a} is hydrogen and R^{1b} is arylalkyl; R² is an amino acid side chain; R³ is alkyl; D* is acyl; and n is four. In another embodiment, R^{1a} is hydrogen and R^{1b} is heteroaryl; R² is an amino acid side chain; R³ is alkyl; D* is acyl; and n is four. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is acyl; and n is four. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is acyl; and n is four. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is acyl; and n is four. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is acyl; and n is four. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4, 5-, or 6-membered heterocyclyl; D* is acyl; and n is four. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is acyl; and n is four. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is acyl; and n is four. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene, further bonded to R^{1a} to form the 4-, 5-, or 6-membered heterocyclyl; D* is acyl; and n is four. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is acyl; and n is four. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is acyl; and n is four. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is acyl; and n is four. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is acyl; and n is four. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is acyl; and n is four. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is acyl; and n is four. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is acyl; and n is four. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is acyl; and n is four. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is acyl; and n is four. In another embodiment, R^{1a} is hydrogen and R^{1b} is hydrogen; R² is hydrogen; R³ is hydrogen; D* is a residue of a biologically active compound; and n is four. In another embodiment, R^{1a} is hydrogen and R^{1b} is alkyl; R² is hydrogen; R³ is hydrogen; D* is a residue of a biologically active compound; and n is four. In another embodiment, R^{1a} is hydrogen and R^{1b} is alkoxy; R² is hydrogen; R³ is hydrogen; D* is a residue of a biologically active compound; and n is four. In another embodiment, R^{1a} is hydrogen and R^{1b} is alkenyl; R² is hydrogen; R³ is hydrogen; D* is a residue of a biologically active compound; and n is four. In another embodiment, R^{1a} is hydrogen and R^{1b} is alkynyl; R² is hydrogen; R³ is hydrogen; D* is a residue of a biologically active compound; and n is four. In another embodiment, R^{1a} is hydrogen and R^{1b} is aryl; R² is hydrogen; R³ is hydrogen; D* is a residue of a biologically active compound; and n is four. In another embodiment, R^{1a} is hydrogen and R^{1b} is arylalkyl; R² is hydrogen; R³ is hydrogen; D* is a residue of a biologically active compound; and n is four. In another embodiment, R^{1a} is hydrogen and R^{1b} is heteroaryl; R² is hydrogen; R³ is hydrogen; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is a residue of a biologically active compound; and n is four. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is a residue of a biologically active compound; and n is four. In another embodiment, R^{1a} is hydrogen and R^{1b} is hydrogen; R² is an amino acid side chain; R³ is hydrogen; D* is a residue of a biologically active compound; and n is four. In another embodiment, R^{1a} is hydrogen and R^{1b} is alkyl; R² is amino acid side chain; R³ is hydrogen; D* is a residue of a biologically active compound; and n is four. In another embodiment, R^{1a} is hydrogen and R^{1b} is alkoxy; R² is an amino acid side chain; R³ is hydrogen; D* is a residue of a biologically active compound; and n is four. In another embodiment, R^{1a} is hydrogen and R^{1b} is alkenyl; R² is an amino acid side chain; R³ is hydrogen; D* is a residue of a biologically active compound; and n is four. In another embodiment, R^{1a} is hydrogen and R^{1b} is alkynyl; R² is an amino acid side chain; R³ is hydrogen; D* is a residue of a biologically active compound; and n is four. In another embodiment, R^{1a} is hydrogen and R^{1b} is aryl; R² is an amino acid side chain; R³ is hydrogen; D* is a residue of a biologically active compound; and n is four. In another embodiment, R^{1a} is hydrogen and R^{1b} is arylalkyl; R² is an amino acid side chain; R³ is hydrogen; D* is a residue of a biologically active compound; and n is four. In another embodiment, R^{1a} is hydrogen and R^{1b} is heteroaryl; R² is an amino acid side chain; R³ is hydrogen; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is a residue of a biologically active compound; and n is four. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is a residue of a biologically active compound; and n is four. In another embodiment, R^{1a} is hydrogen and R^{1b} is hydrogen; R² is hydrogen; R³ is alkyl; D* is a residue of a biologically active compound; and n is four. In another embodiment, R^{1a} is hydrogen and R^{1b} is alkyl; R² is hydrogen; R³ is alkyl; D* is a residue of a biologically active compound; and n is four. In another embodiment, R^{1a} is hydrogen and R^{1b} is alkoxy; R² is hydrogen; R³ is alkyl; D* is a residue of a biologically active compound; and n is four. In another embodiment, R^{1a} is hydrogen and R^{1b} is alkenyl; R² is hydrogen; R³ is alkyl; D* is a residue of a biologically active compound; and n is four. In another embodiment, R^{1a} is hydrogen and R^{1b} is alkynyl; R² is hydrogen; R³ is alkyl; D* is a residue of a biologically active compound; and n is four. In another embodiment, R^{1a} is hydrogen and R^{1b} is aryl; R² is hydrogen; R³ is alkyl; D* is a residue of a biologically active compound; and n is four. In another embodiment, R^{1a} is hydrogen and R^{1b} is arylalkyl; R² is hydrogen; R³ is alkyl; D* is a residue of a biologically active compound; and n is four. In another embodiment, R^{1a} is hydrogen and R^{1b} is heteroaryl; R² is hydrogen; R³ is alkyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is a residue of a biologically active compound; and n is four. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is a residue of a biologically active compound; and n is four. In another embodiment, R^{1a} is hydrogen and R^{1b} is hydrogen; R² is an amino acid side chain; R³ is alkyl; D* is a residue of a biologically active compound; and n is four. In another embodiment, R^{1a} is hydrogen and R^{1b} is alkyl; R² is amino acid side chain; R³ is alkyl; D* is a residue of a biologically active compound; and n is four. In another embodiment, R^{1a} is hydrogen and R^{1b} is alkoxy; R² is an amino acid side chain; R³ is alkyl; D* is a residue of a biologically active compound; and n is four. In another embodiment, R^{1a} is hydrogen and R^{1b} is alkenyl; R² is an amino acid side chain; R³ is alkyl; D* is a residue of a biologically active compound; and n is four. In another embodiment, R^{1a} is hydrogen and R^{1b} is alkynyl; R² is an amino acid side chain; R³ is alkyl; D* is a residue of a biologically active compound; and n is four. In another embodiment, R^{1a} is hydrogen and R^{1b} is aryl; R² is an amino acid side chain; R³ is alkyl; D* is a residue of a biologically active compound; and n is four. In another embodiment, R^{1a} is hydrogen and R^{1b} is arylalkyl; R² is an amino acid side chain; R³ is alkyl; D* is a residue of a biologically active compound; and n is four. In another embodiment, R^{1a} is hydrogen and R^{1b} is heteroaryl; R² is an amino acid side chain; R³ is alkyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is a residue of a biologically active compound; and n is four. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene, further bonded to R^{1a} to form the 4-, 5-, or 6-membered heterocyclyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4-, 5-, or 6-membered heterocyclyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is a residue of a biologically active compound; and n is four. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is a residue of a biologically active compound; and n is four. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is a residue of a biologically active compound; and n is four. In any of the embodiments in this paragraph, R⁴ is hydrogen. In any of the embodiments in this paragraph, R⁴ is alkyl.

In another embodiment, R^{1a} is hydrogen and R^{1b} is hydrogen; R² is hydrogen; R³ is hydrogen; D* is hydrogen; and n is five. In another embodiment, R^{1a} is hydrogen and R^{1b} is alkyl; R² is hydrogen; R³ is hydrogen; D* is hydrogen; and n is five. In another embodiment, R^{1a} is hydrogen and R^{1b} is alkoxy; R² is hydrogen; R³ is hydrogen; D* is hydrogen; and n is five. In another embodiment, R^{1a} is hydrogen and R^{1b} is alkenyl; R² is hydrogen; R³ is hydrogen; D* is hydrogen; and n is five. In another embodiment, R^{1a} is hydrogen and R^{1b} is alkynyl; R² is hydrogen; R³ is hydrogen; D* is hydrogen; and n is five. In another embodiment, R^{1a} is hydrogen and R^{1b} is aryl; R² is hydrogen; R³ is hydrogen; D* is hydrogen; and n is five. In another embodiment, R^{1a} is hydrogen and R^{1b} is arylalkyl; R² is hydrogen; R³ is hydrogen; D* is hydrogen; and n is five. In another embodiment, R^{1a} is hydrogen and R^{1b} is heteroaryl; R² is hydrogen; R³ is hydrogen; D* is hydrogen; and n is five. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is hydrogen; and n is five. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is hydrogen; and n is five. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is hydrogen; and n is five. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is hydrogen; and n is five. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is hydrogen; and n is five. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is hydrogen; and n is five. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is hydrogen; and n is five. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is hydrogen; and n is five. In another embodiment, R^{1a} is hydrogen and R^{1b} is hydrogen; R² is an amino acid side chain; R³ is hydrogen; D* is hydrogen; and n is five. In another embodiment, R^{1a} is hydrogen and R^{1b} is alkyl; R² is amino acid side chain; R³ is hydrogen; D* is hydrogen; and n is five. In another embodiment, R^{1a} is hydrogen and R^{1b} is alkoxy; R² is an amino acid side chain; R³ is hydrogen; D* is hydrogen; and n is five. In another embodiment, R^{1a} is hydrogen and R^{1b} is alkenyl; R² is an amino acid side chain; R³ is hydrogen; D* is hydrogen; and n is five. In another embodiment, R^{1a} is hydrogen and R^{1b} is alkynyl; R² is an amino acid side chain; R³ is hydrogen; D* is hydrogen; and n is five. In another embodiment, R^{1a} is hydrogen and R^{1b} is aryl; R² is an amino acid side chain; R³ is hydrogen; D* is hydrogen; and n is five. In another embodiment, R^{1a} is hydrogen and R^{1b} is arylalkyl; R² is an amino acid side chain; R³ is hydrogen; D* is hydrogen; and n is five. In another embodiment, R^{1a} is hydrogen and R^{1b} is heteroaryl; R² is an amino acid side chain; R³ is hydrogen; D* is hydrogen; and n is five. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is hydrogen; and n is five. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is hydrogen; and n is five. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is hydrogen; and n is five. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is hydrogen; and n is five. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is hydrogen; and n is five. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is hydrogen; and n is five. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is hydrogen; and n is five. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is hydrogen; and n is five. In another embodiment, R^{1a} is hydrogen and R^{1b} is hydrogen; R² is hydrogen; R³ is alkyl; D* is hydrogen; and n is five. In another embodiment, R^{1a} is hydrogen and R^{1b} is alkyl; R² is hydrogen; R³ is alkyl; D* is hydrogen; and n is five. In another embodiment, R^{1a} is hydrogen and R^{1b} is alkoxy; R² is hydrogen; R³ is alkyl; D* is hydrogen; and n is five. In another embodiment, R^{1a} is hydrogen and R^{1b} is alkenyl; R² is hydrogen; R³ is alkyl; D* is hydrogen; and n is five. In another embodiment, R^{1a} is hydrogen and R^{1b} is alkynyl; R² is hydrogen; R³ is alkyl; D* is hydrogen; and n is five. In another embodiment, R^{1a} is hydrogen and R^{1b} is aryl; R² is hydrogen; R³ is alkyl; D* is hydrogen; and n is five. In another embodiment, Ria is hydrogen and R^{1b} is arylalkyl; R² is hydrogen; R³ is alkyl; D* is hydrogen; and n is five. In another embodiment, R^{1a} is hydrogen and R^{1b} is heteroaryl; R² is hydrogen; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is hydrogen; and n is five. In any of the embodiments in this paragraph, R⁴ is hydrogen. In any of the embodiments in this paragraph, R⁴ is alkyl.

In another embodiment, R^{1a} is hydrogen and R^{1b} is hydrogen; R² is an amino acid side chain; R³ is alkyl; D* is hydrogen; and n is five. In another embodiment, R^{1a} is hydrogen and R^{1b} is alkyl; R² is amino acid side chain; R³ is alkyl; D* is hydrogen; and n is five. In another embodiment, R^{1a} is hydrogen and R^{1b} is alkoxy; R² is an amino acid side chain; R³ is alkyl; D* is hydrogen; and n is five. In another embodiment, R^{1a} is hydrogen and R^{1b} is alkenyl; R² is an amino acid side chain; R³ is alkyl; D* is hydrogen; and n is five. In another embodiment, R^{1a} is hydrogen and R^{1b} is alkynyl; R² is an amino acid side chain; R³ is alkyl; D* is hydrogen; and n is five. In another embodiment, R^{1a} is hydrogen and R^{1b} is aryl; R² is an amino acid side chain; R³ is alkyl; D* is hydrogen; and n is five. In another embodiment, R^{1a} is hydrogen and R^{1b} is arylalkyl; R² is an amino acid side chain; R³ is alkyl; D* is hydrogen; and n is five. In another embodiment, R^{1a} is hydrogen and R^{1b} is heteroaryl; R² is an amino acid side chain; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is hydrogen; and n is five. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is hydrogen; and n is five. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is hydrogen; and n is five. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is hydrogen; and n is five. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4-, 5-, or 6-membered heterocyclyl; D* is hydrogen; and n is five. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is hydrogen; and n is five. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is hydrogen; and n is five. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is hydrogen; and n is five. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is hydrogen; and n is five. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is hydrogen; and n is five. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is hydrogen; and n is five. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is hydrogen; and n is five. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is hydrogen; and n is five. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is hydrogen; and n is five. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is hydrogen; and n is five. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is hydrogen; and n is five. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is hydrogen; and n is five. In another embodiment, R^{1a} is hydrogen and R^{1b} is hydrogen; R² is hydrogen; R³ is hydrogen; D* is acyl; and n is five. In another embodiment, R^{1a} is hydrogen and R^{1b} is alkyl; R² is hydrogen; R³ is hydrogen; D* is acyl; and n is five. In another embodiment, R^{1a} is hydrogen and R^{1b} is alkoxy; R² is hydrogen; R³ is hydrogen; D* is acyl; and n is five. In another embodiment, R^{1a} is hydrogen and R^{1b} is alkenyl; R² is hydrogen; R³ is hydrogen; D* is acyl; and n is five. In another embodiment, R^{1a} is hydrogen and R^{1b} is alkynyl; R² is hydrogen; R³ is hydrogen; D* is acyl; and n is five. In another embodiment, R^{1a} is hydrogen and R^{1b} is aryl; R² is hydrogen; R³ is hydrogen; D* is acyl; and n is five. In another embodiment, R^{1a} is hydrogen and R^{1b} is arylalkyl; R² is hydrogen; R³ is hydrogen; D* is acyl; and n is five. In another embodiment, R^{1a} is hydrogen and R^{1b} is heteroaryl; R² is hydrogen; R³ is hydrogen; D* is acyl; and n is five. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is acyl; and n is five. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is acyl; and n is five. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is acyl; and n is five. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is acyl; and n is five. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is acyl; and n is five. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is acyl; and n is five. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is acyl; and n is five. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is acyl; and n is five. In another embodiment, R^{1a} is hydrogen and R^{1b} is hydrogen; R² is an amino acid side chain; R³ is hydrogen; D* is acyl; and n is five. In another embodiment, R^{1a} is hydrogen and R^{1b} is alkyl; R² is amino acid side chain; R³ is hydrogen; D* is acyl; and n is five. In another embodiment, R^{1a} is hydrogen and R^{1b} is alkoxy; R² is an amino acid side chain; R³ is hydrogen; D* is acyl; and n is five. In another embodiment, R^{1a} is hydrogen and R^{1b} is alkenyl; R² is an amino acid side chain; R³ is hydrogen; D* is acyl; and n is five. In another embodiment, R^{1a} is hydrogen and R^{1b} is alkynyl; R² is an amino acid side chain; R³ is hydrogen; D* is acyl; and n is five. In another embodiment, R^{1a} is hydrogen and R^{1b} is aryl; R² is an amino acid side chain; R³ is hydrogen; D* is acyl; and n is five. In another embodiment, R^{1a} is hydrogen and R^{1b} is arylalkyl; R² is an amino acid side chain; R³ is hydrogen; D* is acyl; and n is five. In another embodiment, R^{1a} is hydrogen and R^{1b} is heteroaryl; R² is an amino acid side chain; R³ is hydrogen; D* is acyl; and n is five. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is acyl; and n is five. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is acyl; and n is five. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is acyl; and n is five. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is acyl; and n is five. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is acyl; and n is five. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is acyl; and n is five. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is acyl; and n is five. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is acyl; and n is five. In another embodiment, R^{1a} is hydrogen and R^{1b} is hydrogen; R² is hydrogen; R³ is alkyl; D* is acyl; and n is five. In another embodiment, R^{1a} is hydrogen and R^{1b} is alkyl; R² is hydrogen; R³ is alkyl; D* is acyl; and n is five. In another embodiment, R^{1a} is hydrogen and R^{1b} is alkoxy; R² is hydrogen; R³ is alkyl; D* is acyl; and n is five. In another embodiment, R^{1a} is hydrogen and R^{1b} is alkenyl; R² is hydrogen; R³ is alkyl; D* is acyl; and n is five. In another embodiment, R^{1a} is hydrogen and R^{1b} is alkynyl; R² is hydrogen; R³ is alkyl; D* is acyl; and n is five. In another embodiment, R^{1a} is hydrogen and R^{1b} is aryl; R² is hydrogen; R³ is alkyl; D* is acyl; and n is five. In another embodiment, R^{1a} is hydrogen and R^{1b} is arylalkyl; R² is hydrogen; R³ is alkyl; D* is acyl; and n is five. In another embodiment, R^{1a} is hydrogen and R^{1b} is heteroaryl; R² is hydrogen; R³ is alkyl; D* is acyl; and n is five. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is acyl; and n is five. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is acyl; and n is five. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is acyl; and n is five. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is acyl; and n is five. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is acyl; and n is five. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is acyl; and n is five. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is acyl; and n is five. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is acyl; and n is five. In another embodiment, R^{1a} is hydrogen and R^{1b} is hydrogen; R² is an amino acid side chain; R³ is alkyl; D* is acyl; and n is five. In another embodiment, R^{1a} is hydrogen and R^{1b} is alkyl; R² is amino acid side chain; R³ is alkyl; D* is acyl; and n is five. In another embodiment, R^{1a} is hydrogen and R^{1b} is alkoxy; R² is an amino acid side chain; R³ is alkyl; D* is acyl; and n is five. In another embodiment, R^{1a} is hydrogen and R^{1b} is alkenyl; R² is an amino acid side chain; R³ is alkyl; D* is acyl; and n is five. In another embodiment, R^{1a} is hydrogen and R^{1b} is alkynyl; R² is an amino acid side chain; R³ is alkyl; D* is acyl; and n is five. In another embodiment, R^{1a} is hydrogen and R^{1b} is aryl; R² is an amino acid side chain; R³ is alkyl; D* is acyl; and n is five. In another embodiment, R^{1a} is hydrogen and R^{1b} is arylalkyl; R² is an amino acid side chain; R³ is alkyl; D* is acyl; and n is five. In another embodiment, R^{1a} is hydrogen and R^{1b} is heteroaryl; R² is an amino acid side chain; R³ is alkyl; D* is acyl; and n is five. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is acyl; and n is five. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is acyl; and n is five. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is acyl; and n is five. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is acyl; and n is five. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is acyl; and n is five. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is acyl; and n is five. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is acyl; and n is five. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is acyl; and n is five. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is acyl; and n is five. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is acyl; and n is five. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is acyl; and n is five. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4, 5-, or 6-membered heterocyclyl; D* is acyl; and n is five. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is acyl; and n is five. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is acyl; and n is five. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is acyl; and n is five. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is acyl; and n is five. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is acyl; and n is five. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is acyl; and n is five. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is acyl; and n is five. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is acyl; and n is five. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is acyl; and n is five. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is acyl; and n is five. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is acyl; and n is five. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is acyl; and n is five. In another embodiment, R^{1a} is hydrogen and R^{1b} is hydrogen; R² is hydrogen; R³ is hydrogen; D* is a residue of a biologically active compound; and n is five. In another embodiment, R^{1a} is hydrogen and R^{1b} is alkyl; R² is hydrogen; R³ is hydrogen; D* is a residue of a biologically active compound; and n is five. In another embodiment, R^{1a} is hydrogen and R^{1b} is alkoxy; R² is hydrogen; R³ is hydrogen; D* is a residue of a biologically active compound; and n is five. In another embodiment, R^{1a} is hydrogen and R^{1b} is alkenyl; R² is hydrogen; R³ is hydrogen; D* is a residue of a biologically active compound; and n is five. In another embodiment, R^{1a} is hydrogen and R^{1b} is alkynyl; R² is hydrogen; R³ is hydrogen; D* is a residue of a biologically active compound; and n is five. In another embodiment, R^{1a} is hydrogen and R^{1b} is aryl; R² is hydrogen; R³ is hydrogen; D* is a residue of a biologically active compound; and n is five. In another embodiment, R^{1a} is hydrogen and R^{1b} is arylalkyl; R² is hydrogen; R³ is hydrogen; D* is a residue of a biologically active compound; and n is five. In any of the embodiments in this paragraph, R⁴ is hydrogen. In any of the embodiments in this paragraph, R⁴ is alkyl.

In another embodiment, R^{1a} is hydrogen and R^{1b} is heteroaryl; R² is hydrogen; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is hydrogen; R² is hydrogen; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkyl; R² is hydrogen; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkenyl; R² is hydrogen; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkynyl; R² is hydrogen; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is aryl; R² is hydrogen; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is hydrogen; D* is a residue of a biologically active compound; and n is five. In another embodiment, R^{1a} is hydrogen and R^{1b} is hydrogen; R² is an amino acid side chain; R³ is hydrogen; D* is a residue of a biologically active compound; and n is five. In another embodiment, R^{1a} is hydrogen and R^{1b} is alkyl; R² is amino acid side chain; R³ is hydrogen; D* is a residue of a biologically active compound; and n is five. In another embodiment, R^{1a} is hydrogen and R^{1b} is alkoxy; R² is an amino acid side chain; R³ is hydrogen; D* is a residue of a biologically active compound; and n is five. In another embodiment, R^{1a} is hydrogen and R^{1b} is alkenyl; R² is an amino acid side chain; R³ is hydrogen; D* is a residue of a biologically active compound; and n is five. In another embodiment, R^{1a} is hydrogen and R^{1b} is alkynyl; R² is an amino acid side chain; R³ is hydrogen; D* is a residue of a biologically active compound; and n is five. In another embodiment, R^{1a} is hydrogen and R^{1b} is aryl; R² is an amino acid side chain; R³ is hydrogen; D* is a residue of a biologically active compound; and n is five. In any of the embodiments in this paragraph, R⁴ is hydrogen. In any of the embodiments in this paragraph, R⁴ is alkyl.

In another embodiment, R^{1a} is hydrogen and R^{1b} is arylalkyl; R² is an amino acid side chain; R³ is hydrogen; D* is a residue of a biologically active compound; and n is five. In another embodiment, R^{1a} is hydrogen and R^{1b} is heteroaryl; R² is an amino acid side chain; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is hydrogen; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is hydrogen; D* is a residue of a biologically active compound; and n is five. In another embodiment, R^{1a} is hydrogen and R^{1b} is hydrogen; R² is hydrogen; R³ is alkyl; D* is a residue of a biologically active compound; and n is five. In another embodiment, R^{1a} is hydrogen and R^{1b} is alkyl; R² is hydrogen; R³ is alkyl; D* is a residue of a biologically active compound; and n is five. In another embodiment, R^{1a} is hydrogen and R^{1b} is alkoxy; R² is hydrogen; R³ is alkyl; D* is a residue of a biologically active compound; and n is five. In another embodiment, R^{1a} is hydrogen and R^{1b} is alkenyl; R² is hydrogen; R³ is alkyl; D* is a residue of a biologically active compound; and n is five. In any of the embodiments in this paragraph, R⁴ is hydrogen. In any of the embodiments in this paragraph, R⁴ is alkyl.

In another embodiment, R^{1a} is hydrogen and R^{1b} is alkynyl; R² is hydrogen; R³ is alkyl; D* is a residue of a biologically active compound; and n is five. In another embodiment, R^{1a} is hydrogen and R^{1b} is aryl; R² is hydrogen; R³ is alkyl; D* is a residue of a biologically active compound; and n is five. In another embodiment, R^{1a} is hydrogen and R^{1b} is arylalkyl; R² is hydrogen; R³ is alkyl; D* is a residue of a biologically active compound; and n is five. In another embodiment, R^{1a} is hydrogen and R^{1b} is heteroaryl; R² is hydrogen; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is aryl; R² is hydrogen; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkyl; D* is a residue of a biologically active compound; and n is five. In another embodiment, R^{1a} is hydrogen and R^{1b} is hydrogen; R² is an amino acid side chain; R³ is alkyl; D* is a residue of a biologically active compound; and n is five. In another embodiment, R^{1a} is hydrogen and R^{1b} is alkyl; R² is amino acid side chain; R³ is alkyl; D* is a residue of a biologically active compound; and n is five. In another embodiment, R^{1a} is hydrogen and R^{1b} is alkoxy; R² is an amino acid side chain; R³ is alkyl; D* is a residue of a biologically active compound; and n is five. In any of the embodiments in this paragraph, R⁴ is hydrogen. In any of the embodiments in this paragraph, R⁴ is alkyl.

In another embodiment, R^{1a} is hydrogen and R^{1b} is alkenyl; R² is an amino acid side chain; R³ is alkyl; D* is a residue of a biologically active compound; and n is five. In another embodiment, R^{1a} is hydrogen and R^{1b} is alkynyl; R² is an amino acid side chain; R³ is alkyl; D* is a residue of a biologically active compound; and n is five. In another embodiment, R^{1a} is hydrogen and R^{1b} is aryl; R² is an amino acid side chain; R³ is alkyl; D* is a residue of a biologically active compound; and n is five. In another embodiment, R^{1a} is hydrogen and R^{1b} is arylalkyl; R² is an amino acid side chain; R³ is alkyl; D* is a residue of a biologically active compound; and n is five. In another embodiment, R^{1a} is hydrogen and R^{1b} is heteroaryl; R² is an amino acid side chain; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkyl; D* is a residue of a biologically active compound; and n is five. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is hydrogen; R² is hydrogen; R³ is alkylene further bonded to R^{1a} to form the 4-, 5-, or 6-membered heterocyclyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkyl; R² is hydrogen; R³ is alkylene further bonded to R^{1a} to form the 4-, 5-, or 6-membered heterocyclyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkoxyl; R² is hydrogen; R³ is alkylene further bonded to R^{1a} to form the 4-, 5-, or 6-membered heterocyclyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkenyl; R² is hydrogen; R³ is alkylene further bonded to R^{1a} to form the 4-, 5-, or 6-membered heterocyclyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkynyl; R² is hydrogen; R³ is alkylene further bonded to R^{1a} to form the 4-, 5-, or 6-membered heterocyclyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is aryl; R² is hydrogen; R³ is alkylene further bonded to R^{1a} to form the 4-, 5-, or 6-membered heterocyclyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is arylalkyl; R² is hydrogen; R³ is alkylene further bonded to R^{1a} to form the 4-, 5-, or 6-membered heterocyclyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is heteroaryl; R² is hydrogen; R³ is alkylene further bonded to R^{1a} to form the 4-, 5-, or 6-membered heterocyclyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is hydrogen; R² is an amino acid side chain; R³ is alkylene further bonded to R^{1a} to form the 4-, 5-, or 6-membered heterocyclyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkyl; R² is an amino acid side chain; R³ is alkylene further bonded to R^{1a} to form the 4-, 5-, or 6-membered heterocyclyl; D* is a residue of a biologically active compound; and n is five. In any of the embodiments in this paragraph, R⁴ is hydrogen. In any of the embodiments in this paragraph, R⁴ is alkyl.

In another embodiment, R^{1a} is alkylene, wherein the alkylene is further bonded to R³ to form a 4-, 5-, or 6-membered heterocyclyl; and R^{1b} is alkoxyl; R² is an amino acid side chain; R³ is alkylene further bonded to R^{1a} to form the 4-, 5-, or 6-membered heterocyclyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkenyl; R² is an amino acid side chain; R³ is alkylene further bonded to R^{1a} to form the 4-, 5-, or 6-membered heterocyclyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is alkynyl; R² is an amino acid side chain; R³ is alkylene further bonded to R^{1a} to form the 4-, 5-, or 6-membered heterocyclyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is aryl; R² is an amino acid side chain; R³ is alkylene further bonded to R^{1a} to form the 4-, 5-, or 6-membered heterocyclyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is arylalkyl; R² is an amino acid side chain; R³ is alkylene further bonded to R^{1a} to form the 4-, 5-, or 6-membered heterocyclyl; D* is a residue of a biologically active compound; and n is five. In another embodiment, R^{1a} is alkylene, wherein the alkylene is further bonded to R³ to form a 4-, 5-, or 6-membered heterocyclyl; and R^{1b} is heteroaryl; R² is an amino acid side chain; R³ is alkylene further bonded to R^{1a} to form the 4-, 5-, or 6-membered heterocyclyl; D* is a residue of a biologically active compound; and n is five. In any of the embodiments in this paragraph, R⁴ is hydrogen. In any of the embodiments in this paragraph, R⁴ is alkyl.

In certain embodiments of Formula I and/or Ib, R^{1a} and R^{1b} are each hydrogen. In another embodiment of Formula I and/or Ib, n is two. In another embodiment of Formula I and/or Ib, n is two and R² is hydrogen or methyl. In another embodiment of Formula I and/or Ib, n is two, R² is hydrogen or methyl, R³ is hydrogen, and D* is a residue of a biologically active compound comprising hydroxyl. In another embodiment of Formula I and/or Ib, the compound is selected from the group consisting of:

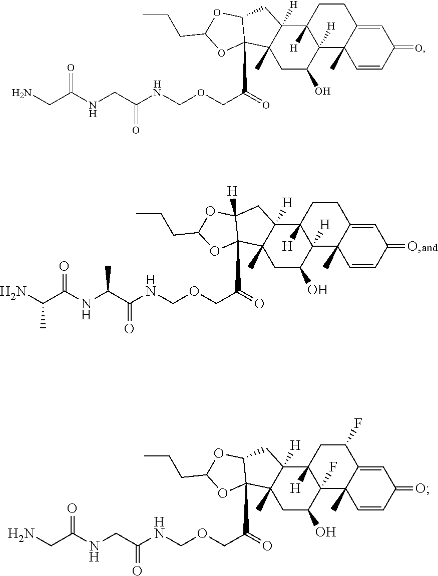

or a pharmaceutically acceptable salt thereof. In certain embodiments within this paragraph, all diastereomers are contemplated. For example, in one embodiment, the stereochemistry at the acetal is undefined or racemic. By way of further example, in one embodiment, the stereochemistry at the acetal is (R)-. By way of further example, in one embodiment, the stereochemistry at the acetal is (S)-. By way of further example, in one embodiment, the stereochemistry at the acetal is (R)- in excess of (S)-. By way of further example, in one embodiment, the stereochemistry at the acetal is (S)- in excess of (R)-.

In certain embodiments of Formula I and/or Ib, R^{1a} and R^{1b} are each hydrogen. In another embodiment of Formula I and/or Ib, n is one. In another embodiment of Formula I and/or Ib, R² is hydrogen, methyl, or —CH₂Ph. In another embodiment of Formula I and/or Ib, R³ is hydrogen. In another embodiment of Formula I and/or Ib, D* is a residue of a biologically active compound comprising hydroxyl. In another embodiment of Formula I and/or Ib, the compound is selected from the group consisting of:

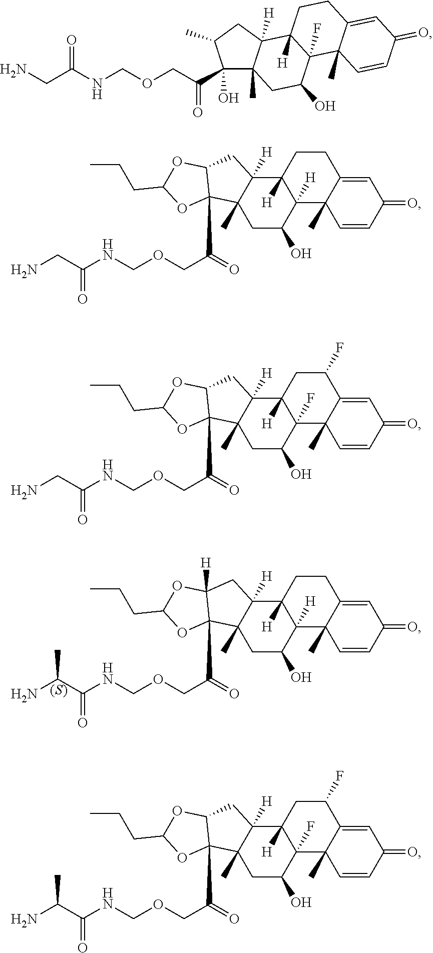

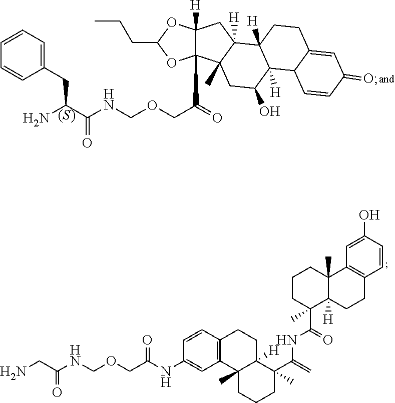

or a pharmaceutically acceptable salt thereof. In certain embodiments within this paragraph, all diastereomers are contemplated. For example, in one embodiment, the stereochemistry at the acetal is undefined or racemic. By way of further example, in one embodiment, the stereochemistry at the acetal is (R)-. By way of further example, in one embodiment, the stereochemistry at the acetal is (S)-. By way of further example, in one embodiment, the stereochemistry at the acetal is (R)- in excess of (S)-. By way of further example, in one embodiment, the stereochemistry at the acetal is (S)- in excess of (R)-.

In certain embodiments of Formula I and/or Ib, R^{1a} and R^{1b} are each hydrogen. In another embodiment of Formula I and/or Ib, n is one. In another embodiment of Formula I and/or Ib, R² is hydrogen or methyl. In another embodiment of Formula I and/or Ib, R³ is alkyl. In another embodiment of Formula I and/or Ib, D* is a residue of a biologically active compound comprising hydroxyl. In another embodiment of Formula I and/or Ib, the compound is selected from the group consisting of

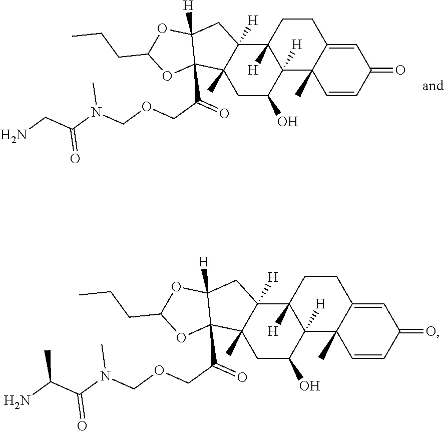

or a pharmaceutically acceptable salt thereof. In certain embodiments within this paragraph, all diastereomers are contemplated. For example, in one embodiment, the stereochemistry at the acetal is undefined or racemic. By way of further example, in one embodiment, the stereochemistry at the acetal is (R)-. By way of further example, in one embodiment, the stereochemistry at the acetal is (S)-. By way of further example, in one embodiment, the stereochemistry at the acetal is (R)- in excess of (S)-. By way of further example, in one embodiment, the stereochemistry at the acetal is (S)- in excess of (R)-.

In certain embodiments of Formula I, R^{1a} is alkyl or arylalkyl, and R^{1b} is hydrogen. In another embodiment of Formula I, n is one. In another embodiment of Formula I, R² is hydrogen. In another embodiment of Formula I, R³ is hydrogen. In another embodiment of Formula I, D* is a residue of a biologically active compound comprising hydroxyl. In another embodiment of Formula I, the compound is selected from the group consisting of

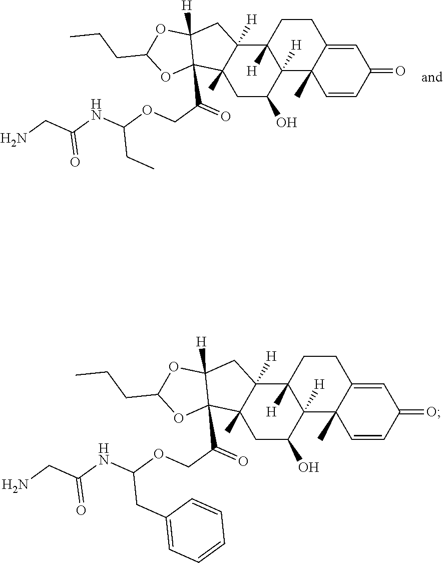

or a pharmaceutically acceptable salt thereof. In certain embodiments within this paragraph, all diastereomers are contemplated. For example, in one embodiment, the stereochemistry at the hemiaminal ether (or hemiaminal, or N-acyl-N,O-acetal, wherein each name for this functional group is used interchangeably throughout this disclosure) is undefined or racemic. By way of further example, in one embodiment, the stereochemistry at the hemiaminal ether is (R)-. By way of further example, in one embodiment, the stereochemistry at the hemiaminal ether is (S)-. By way of further example, in one embodiment, the stereochemistry at the hemiaminal ether is (R)- in excess of (S)-. By way of further example, in one embodiment, the stereochemistry at the hemiaminal ether is (S)- in excess of (R)-. For example, in one embodiment, the stereochemistry at the acetal is undefined or racemic. By way of further example, in one embodiment, the stereochemistry at the acetal is (R)-. By way of further example, in one embodiment, the stereochemistry at the acetal is (S)-. By way of further example, in one embodiment, the stereochemistry at the acetal is (R)- in excess of (S)-. By way of further example, in one embodiment, the stereochemistry at the acetal is (S)- in excess of (R)-.

In certain embodiments of Formula I, R^{1a} is alkylene, where the alkylene is further bonded to R³ to form a 4-, 5-, or 6-membered heterocyclyl; R^{1b} is hydrogen; and R³ is alkylene further bonded to R^{1a} to form the 4-, 5-, or 6-membered heterocyclyl. Useful 4-membered heterocyclyls include, without limitation, optionally substituted aziridine. Useful 5-membered heterocyclyls include, without limitation, optionally substituted pyrrolidine. In one exemplary embodiment, optionally substituted pyrrolidine is indoline or indolinyl. Useful 6-membered heterocyclyls include, without limitation, optionally substituted piperidine. In one exemplary embodiment, optionally substituted piperidine is tetrahydroquinoline or tetrahydroquinolinyl. In another embodiment of Formula I, n is one. In another embodiment of Formula I, R² is hydrogen. In another embodiment of Formula I, D* is a residue of a biologically active compound comprising hydroxyl. In another embodiment of Formula I, the compound is selected from the group consisting of

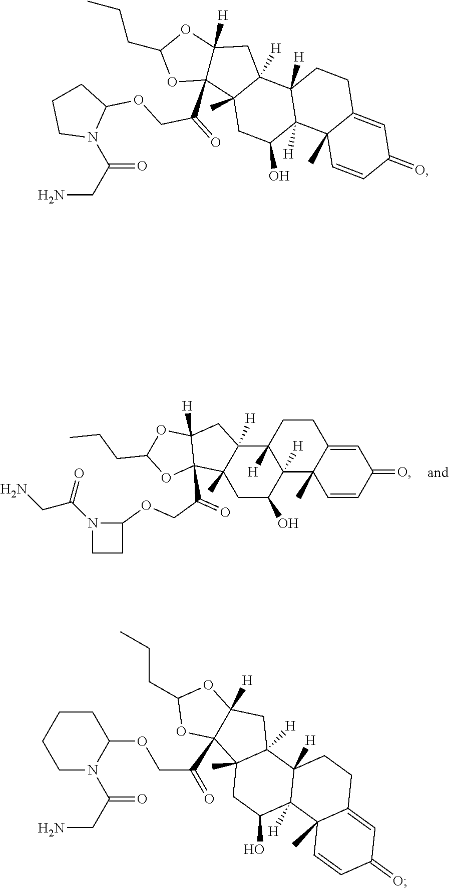

or a pharmaceutically acceptable salt thereof. In certain embodiments within this paragraph, all diastereomers are contemplated. For example, in one embodiment, the stereochemistry at the hemiaminal ether (or hemiaminal, or N-acyl-N,O-acetal, wherein each name for this functional group is used interchangeably throughout this disclosure) is undefined or racemic. By way of further example, in one embodiment, the stereochemistry at the hemiaminal ether is (R)-. By way of further example, in one embodiment, the stereochemistry at the hemiaminal ether is (S)-. By way of further example, in one embodiment, the stereochemistry at the hemiaminal ether is (R)- in excess of (S)-. By way of further example, in one embodiment, the stereochemistry at the hemiaminal ether is (S)- in excess of (R)-. For example, in one embodiment, the stereochemistry at the acetal is undefined or racemic. By way of further example, in one embodiment, the stereochemistry at the acetal is (R)-. By way of further example, in one embodiment, the stereochemistry at the acetal is (S)-. By way of further example, in one embodiment, the stereochemistry at the acetal is (R)- in excess of (S)-. By way of further example, in one embodiment, the stereochemistry at the acetal is (S)- in excess of (R)-.

In certain embodiments, other compounds, prodrugs, payloads, or biologically active compounds comprising hydroxyl are contemplated. Exemplary compounds, prodrugs, or payloads contemplated include, without limitation,

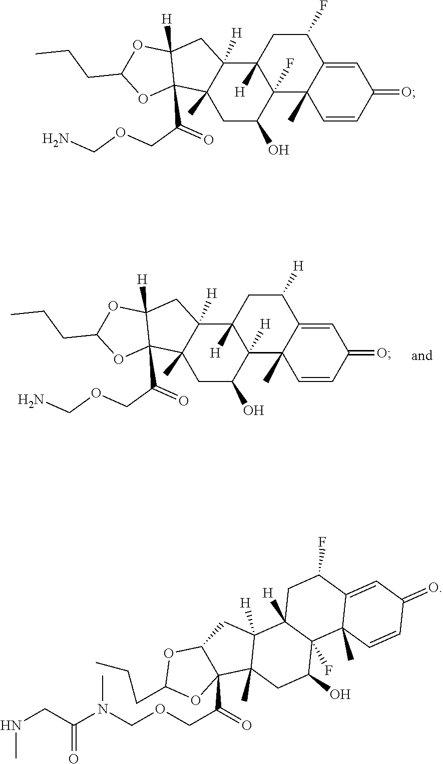

In certain embodiments within this paragraph, all diastereomers are contemplated. For example, in one embodiment, the stereochemistry at the acetal is undefined or racemic. By way of further example, in one embodiment, the stereochemistry at the acetal is (R)-. By way of further example, in one embodiment, the stereochemistry at the acetal is (S)-. By way of further example, in one embodiment, the stereochemistry at the acetal is (R)- in excess of (S)-. By way of further example, in one embodiment, the stereochemistry at the acetal is (S)- in excess of (R)-.

In certain embodiments of Formula I and/or Ib, R^{1a} and R^{1b} are each hydrogen. In another embodiment of Formula I and/or Ib, n is one. In another embodiment of Formula I and/or Ib, R² is alkylene, wherein the alkylene is further bonded to R³ to form a 6-membered heterocyclyl; and R³ is alkylene, wherein the alkylene is further bonded to R² to form the 6-membered heterocyclyl. In another embodiment of Formula I and/or Ib, D* is a residue of a biologically active compound comprising hydroxyl. In another embodiment of Formula I and/or Ib, the compound is

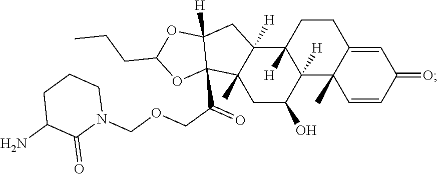

or a pharmaceutically acceptable salt thereof. In certain embodiments within this paragraph, all diastereomers are contemplated. For example, in one embodiment, the stereochemistry at the acetal is undefined or racemic. By way of further example, in one embodiment, the stereochemistry at the acetal is (R)-. By way of further example, in one embodiment, the stereochemistry at the acetal is (S)-. By way of further example, in one embodiment, the stereochemistry at the acetal is (R)- in excess of (S)-. By way of further example, in one embodiment, the stereochemistry at the acetal is (S)- in excess of (R)-.

### Binding Agents

Suitable binding agents for any of the conjugates provided in the instant disclosure include, but are not limited to, antibodies, lymphokines (e.g., IL-2 or IL-3), hormones (e.g., insulin and glucocorticoids), growth factors (e.g., EGF, transferrin, and fibronectin type III), viral receptors, interleukins, or any other cell binding or peptide binding molecules or substances. Binding agents also include, but are not limited to, ankyrin repeat proteins and interferons.

In some embodiments, the binding agent is an antibody or an antigen-binding fragment thereof. The antibody can be in any form known to those of skill in the art. The term “antibody,” as used herein, refers to any antigen-binding molecule or molecular complex comprising at least one complementarity determining region (CDR) that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, CH₁, CH₂ and CH₃. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of disclosed herein, the FRs of the antibodies (or antigen-binding portion thereof) suitable for the compounds herein may be identical to the human germline sequences, or may be naturally or artificially modified. An amino acid consensus sequence may be defined based on a side-by-side analysis of two or more CDRs. The term “antibody”, as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable, standard technique(s) such as proteolytic digestion or recombinant genetic engineering technique(s) involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated CDR such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein. An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L}, or V_{L}-V_{L} dimers. Alternatively, the antigen-binding fragment of an antibody may contain a monomeric V_{H} or V_{L} domain. In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present disclosure using routine techniques available in the art. In certain embodiments described herein, antibodies described herein are human antibodies. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example, in the CDRs and in particular CDR3. However, the term “human antibody”, as used herein, is not intended to include antibodies in which CDR sequences derived from the germline of another mammalian species, such as a mouse, have been grafted onto human framework sequences. The term “human antibody” does not include naturally occurring molecules that normally exist without modification or human intervention/manipulation, in a naturally occurring, unmodified living organism. The antibodies of the invention may, in some embodiments, be recombinant human antibodies. The term “recombinant human antibody”, as used herein, is intended to include all human antibodies that are prepared, expressed, created, or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created, or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo. Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification. The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant disclosure encompasses antibodies having one or more mutations in the hinge, CH₂, or CH₃ region which may be desirable, for example, in production, to improve the yield of the desired antibody form. The antibodies described herein may be isolated antibodies. An “isolated antibody,” as used herein, refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the instant disclosure. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals. The antibodies used herein can comprise one or more amino acid substitutions, insertions, and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure. Antibodies useful for the compounds herein also include antibodies comprising variants of any of the HCVR, LCVR, and/or CDR amino acid sequences disclosed herein having one or more conservative substitutions. The term “epitope” refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

In certain embodiments, the antibody comprises a light chain. In certain embodiments, the light chain is a kappa light chain. In certain embodiments, the light chain is a lambda light chain. In certain embodiments, the antibody comprises a heavy chain. In some embodiments, the heavy chain is an IgA. In some embodiments, the heavy chain is an IgD. In some embodiments, the heavy chain is an IgE. In some embodiments, the heavy chain is an IgG. In some embodiments, the heavy chain is an IgM. In some embodiments, the heavy chain is an IgG1. In some embodiments, the heavy chain is an IgG2. In some embodiments, the heavy chain is an IgG3. In some embodiments, the heavy chain is an IgG4. In some embodiments, the heavy chain is an IgA1. In some embodiments, the heavy chain is an IgA2.

In some embodiments, the antibody is an antibody fragment. In some embodiments, the antibody fragment is an Fv fragment. In some embodiments, the antibody fragment is a Fab fragment. In some embodiments, the antibody fragment is a F(ab′)₂ fragment. In some embodiments, the antibody fragment is a Fab′ fragment. In some embodiments, the antibody fragment is an scFv (sFv) fragment. In some embodiments, the antibody fragment is an scFv-Fc fragment.

In some embodiments, the antibody is a monoclonal antibody. In some embodiments, the antibody is a polyclonal antibody. In some embodiments, the antibody is a bispecific antibody including a first antigen-binding domain (also referred to herein as “D1”), and a second antigen-binding domain (also referred to herein as “D2”).

As used herein, the expression “antigen-binding domain” means any peptide, polypeptide, nucleic acid molecule, scaffold-type molecule, peptide display molecule, or polypeptide-containing construct that is capable of specifically binding a particular antigen of interest (e.g., PRLR or STEAP2). The term “specifically binds” or the like, as used herein, means that the antigen-binding domain forms a complex with a particular antigen characterized by a dissociation constant (K_{D}) of 1 μM or less, and does not bind other unrelated antigens under ordinary test conditions. “Unrelated antigens” are proteins, peptides, or polypeptides that have less than 95% amino acid identity to one another.

Exemplary categories of antigen-binding domains that can be used in the context of the present disclosure include antibodies, antigen-binding portions of antibodies, peptides that specifically interact with a particular antigen (e.g., peptibodies), receptor molecules that specifically interact with a particular antigen, proteins comprising a ligand-binding portion of a receptor that specifically binds a particular antigen, antigen-binding scaffolds (e.g., DARPins, HEAT repeat proteins, ARM repeat proteins, tetratricopeptide repeat proteins, and other scaffolds based on naturally occurring repeat proteins, etc., [see, e.g., Boersma and Pluckthun, 2011, Curr. Opin. Biotechnol. 22:849-857, and references cited therein]), and aptamers or portions thereof.

Methods for determining whether two molecules specifically bind one another are well known in the art and include, for example, equilibrium dialysis, surface plasmon resonance, and the like. For example, an antigen-binding domain, as used in the context of the present disclosure, includes polypeptides that bind a particular antigen (e.g., a target molecule [T] or an internalizing effector protein [E]) or a portion thereof with a K_{D} of less than about 1 μM, less than about 500 nM, less than about 250 nM, less than about 125 nM, less than about 60 nM, less than about 30 nM, less than about 10 nM, less than about 5 nM, less than about 2 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay.

In some embodiments, the antibody is a chimeric antibody. In some embodiments, the antibody is a humanized antibody. In some embodiments, the antibody is a human antibody.

In some embodiments, the antibody is an anti-PSMA, anti-PRLR, anti-MUC16, anti-HER2, or anti-EGFRvIII, or anti-STEAP2 antibody. In some embodiments, the antibody is an anti-PRLR or anti HER2 antibody. In some embodiments, the antibody, or antigen-binding fragment thereof, is anti-STEAP2. In some embodiments, the antibody, or antigen-binding fragment thereof, is anti-PRLR

The antibody can have binding specificity for any antigen deemed suitable to those of skill in the art. In certain embodiments, the antigen is a transmembrane molecule (e.g., receptor). In one embodiment, the antigen is expressed on a tumor. In some embodiments, the binding agents interact with or bind to tumor antigens, including antigens specific for a type of tumor or antigens that are shared, overexpressed, or modified on a particular type of tumor. In one embodiment, the antigen is expressed on solid tumors. Exemplary antigens include, but are not limited to, lipoproteins; alpha1-antitrypsin; a cytotoxic T-lymphocyte associated antigen (CTLA), such as CTLA-4; vascular endothelial growth factor (VEGF); receptors for hormones or growth factors; protein A or D; fibroblast growth factor receptor 2 (FGFR2), EpCAM, GD3, FLT3, PSMA, PSCA, MUC1, MUC16, STEAP, STEAP2, CEA, TENB2, EphA receptors, EphB receptors, folate receptor, FOLRI, mesothelin, cripto, alphavbeta6, integrins, VEGF, VEGFR, EGFR, transferrin receptor, IRTA1, IRTA2, IRTA3, IRTA4, IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80, CD81, CD103, CD105, CD134, CD137, CD138, CD152, or an antibody which binds to one or more tumor-associated antigens or cell-surface receptors disclosed in US Publication No. 2008/0171040 or US Publication No. 2008/0305044 each incorporated in their entirety by reference; erythropoietin; osteoinductive factors; immunotoxins; a bone morphogenetic protein (BMP); T-cell receptors; surface membrane proteins; integrins, such as CD11a, CD11b, CD11c, CD18, an ICAM, VLA-4 and VCAM; a tumor associated antigen such as AFP, ALK, B7H4, BAGE proteins, β-catenin, brc-abl, BRCA1, BORIS, CA9 (carbonic anhydrase IX), caspase-8, CD20, CD40, CD123, CDK4, CEA, CLEC12A, c-kit, cMET, CTLA4, cyclin-B1, CYP 1B1, EGFR, EGFRvIII, endoglin, Epcam, EphA2, ErbB2/Her2, ErbB3/Her3, ErbB4/Her4, ETV6-AML, Fra-1, FOLR1, GAGE proteins, GD2, GD3, GloboH, glypican-3, GM3, gp100, Her2, HLAB-raf, HLA/EBNA1, HLA/k-ras, HLA/MAGE-A3, hTERT, IGF1R, LGR5, LMP2, MAGE proteins, MART-1, mesothelin, ML-IAP, Muc1, Muc16, CA-125, MUM1, NA17, NGEP, NY-BR1, NY-BR62, NY-BR85, NY-ESO1, OX40, p15, p53, PAP, PAX3, PAX5, PCTA-1, PDGFR-α, PDGFR-β, PDGF-A, PDGF-B, PDGF-C, PDGF-D, PLAC1, PRLR, PRAIVIE, PSCA, PSGR, PSMA (FOLH1), RAGE proteins, Ras, RGS5, Rho, SART-1, SART-3, Steap-1, Steap-2, STn, survivin, TAG-72, TGF-β, TMPRSS2, Tn, TNFRSF17, TRP-1, TRP-2, tyrosinase, and uroplakin-3, and fragments of any of the above-listed polypeptides; cell-surface expressed antigens; MUC16; c-MET; molecules such as class A scavenger receptors including scavenger receptor A (SR-A), and other membrane proteins such as B7 family-related member including V-set and Ig domain-containing 4 (VSIG4), Colony stimulating factor 1 receptor (CSF1R), asialoglycoprotein receptor (ASGPR), and Amyloid beta precursor-like protein 2 (APLP-2); macrophage receptor with collagenous structure (MARCO), scavenger receptor with C-type lectin (SRCL), and scavenger receptor A-5 (SCARAS), COLEC12, class B macrophage scavenger receptors including CD36, LIMPII, SRBI, SRBII, class D scavenger receptor CD68, and lysosomal membrane glycoprotein (LAMP), class E scavenger receptor including lectin-like oxidized low density lipoprotein receptor 1 LOX-1 and Dectin-1, class F scavenger receptors including scavenger receptor expressed by endothelial cells-I (SREC-I) and SREC-II as well as multiple epidermal growth factor (EGF)-like domains (MEGF)10, class G scavenger receptor CXC chemokine ligand 16 (CXCL16), class H scavenger receptors including Fasciclin, EGF-like, lamin type EGF-like and link domain-containing scavenger receptor-1 (FEEL-1) and -2 (FEEL-2), class I scavenger receptor CD163, and class J scavenger receptor for advanced glycation end products (RAGE), other C-type lectin superfamily members including DEC205, CD206, Dectin-2, Mincle, DC-SIGN, and DNGR-1, and other membrane proteins such as B7 family-related member including V-set and Ig domain-containing 4 (VSIG4); AXL, BAFFR, BCR-list components, BDCA2, BDCA4, BTLA, BTNL2, BTNL3, BTNL8, BTNL9, C10orf54, CCR1, CCR3, CCR4, CCR5, CCR6, CCR7, CCR9, CCR10, CD168, CD177, CD209, CD209L, CD226, CD248, CD27, CD274, CD276, CD300A, CD45, CD46, CD47, CD48, CD62E, CD68, CD69, CD74, CD79a, CD79b, CD86, CD90.2, CD96, CLEC12B, CLEC7A, CLEC9A, CR1, CR3, CRTAM, CXCR1/2, CXCR4, CXCR5, DDR1, DDR2, DEC-205, DLL4, DR6, FAP, FCamR, FCMR, FcR's, Fire, GITR, HER2, HHLA2, HLA class II, HVEM, ICOSLG, IFNLR1, IL10R1, IL10R2, IL12R, IL13RA1, IL13RA2, IL15R, IL17RA, IL17RB, IL17RC, IL17RE, IL20R1, IL20R2, IL21R, IL22R1, IL22RA, IL23R, IL27R, IL29R, IL2Rg, IL31R, IL36R, IL3RA, IL4R, IL6R, IL5R, IL7R, IL9R, LAG3, LIFR, MAG/Siglec-4, MMR, MSR1, NCR3LG1, NKG2D, NKp30, NKp46, PDCD1, PROKR1, PVR, PVRIG, PVRL2, PVRL3, RELT, SIGIRR, Siglec-1, Siglec-10, Siglec-5, Siglec-6, Siglec-7, Siglec-8, Siglec-9, SIRPA, TACI, TCR-list components/assoc, PTCRA, TCRb, CD3z, TEK, TGFBR1, TGFBR2, TGFBR3, TIGIT, TLR2, TLR4, TNF-α, TROY, TSLPR, TYRO, VLDLR, and VTCN1. In some embodiments, the binding agent is adalimumab or infliximab. In some embodiments, the binding agent is alemtuzumab, muromonab, rituximab, tosituzumab, or agonistic antibodies (where immune stimulation might be part of the intended mechanism of action). In some embodiments, the antigen is PRLR or HER2. In some embodiments, the antigen is STEAP2. In some embodiments the antigen is human STEAP2. In some examples, the MAGE proteins are selected from MAGE-1, -2, -3, -4,-6, and -12. In some examples, the GAGE proteins are selected from GAGE-1 and GAGE-2.

Exemplary antigens also include, but are not limited to, BCMA, SLAMF7, GPNMB, MSR1, and UPK3A. Exemplary antigens also include, but are not limited to, MUC16, STEAP2, and HER2.

In some embodiments, the antigens include MUC16. In some embodiments, the antigens include STEAP2. In some embodiments, the antigens include PSMA. In some embodiments, the antigens include MSR1. In some embodiments, the antigens include HER2. In some embodiments, the antigen is prolactin receptor (PRLR) or prostate-specific membrane antigen (PSMA). In some embodiments, the antigen is MUC16. In some embodiments, the antigens include PSMA. In some embodiments, the antigen is HER2. In some embodiments, the antigen is STEAP2. In some embodiments, the antigen is MSR1.

In certain embodiments, the antibody comprises a glutamine residue at one or more heavy chain positions numbered 295 in the EU numbering system. In the present disclosure, this position is referred to as glutamine 295, or as Gln295, or as Q295. Those of skill will recognize that this is a conserved glutamine residue in the wild type sequence of many antibodies. In other useful embodiments, the antibody can be engineered to comprise a glutamine residue. In certain embodiments, the antibody comprises one or more N297Q mutations. Techniques for modifying an antibody sequence to include a glutamine residue are within the skill of those in the art (see, e.g., Ausubel et al. Current Protoc. Mol. Biol.).

In some embodiments, the antibody, or antigen-binding fragment thereof, conjugated to the linker-payload or payload can be an antibody that targets STEAP2. Suitable anti-STEAP antibodies or antigen-binding fragments thereof include those, for example, in International Publication No. WO 2018/058001 A1, including those comprising amino acid sequences disclosed in Table 1, on page 75 therein. In some embodiments, an anti-STEAP2 antibody is H1H7814N of WO 2018/058001 A1, comprising the CDRs of H1M7814N in the same publication. In some embodiments, an anti-STEAP2 antibody comprises a heavy chain complementarity determining region (HCDR)-1 comprising SEQ ID NO: 2; an HCDR2 comprising SEQ ID NO: 3; an HCDR3 comprising SEQ ID NO: 4; a light chain complementarity determining region (LCDR)-1 comprising SEQ ID NO: 6; an LCDR2 comprising SEQ ID NO: 7; and an LCDR3 comprising SEQ ID NO: 8. In some embodiments, an anti-STEAP2 antibody comprises a heavy chain variable region (HCVR) comprising SEQ ID NO: 1 and a light chain variable region (LCVR) comprising SEQ ID NO: 5. In any of the foregoing embodiments, the anti-STEAP2 antibody can be prepared by site-directed mutagenesis to insert a glutamine residue at a site without resulting in disabled antibody function or binding. For example, in any of the foregoing embodiments, the anti-STEAP2 antibody can comprise an Asn297Gln (N297Q) mutation. Such antibodies having an N297Q mutation can also contain one or more additional naturally occurring glutamine residues in their variable regions, which can be accessible to transglutaminase and therefore capable of conjugation to a payload or a linker-payload (Table A). In certain embodiments, the antibody or antigen-binding fragment thereof comprises three heavy chain complementarity determining regions (HCDR1, HCDR2, and HCDR3) within a heavy chain variable region (HCVR) amino acid sequence of SEQ ID NO:1; and three light chain complementarity determining regions (LCDR1, LCDR2, and LCDR3) within a light chain variable region (LCVR) amino acid sequence of SEQ ID NO:5. In certain embodiments, the antibody or antigen-binding fragment thereof comprises an HCVR amino acid sequence of SEQ ID NO:1; and an LCVR amino acid sequence of SEQ ID NO:5. International Publication No. WO 2018/058001 A1 is hereby incorporated herein by reference in its entirety.

In some embodiments, the antibody, or antigen-binding fragment thereof, conjugated to the linker-payload or payload can be an antibody that targets human prolactin receptor (PRLR). Suitable anti-PRLR antibodies or antigen-binding fragments thereof include those, for example, in International Publication No. WO 2015/026907 A1, including those comprising amino acid sequences disclosed in Table 1, on page 36 therein. In some embodiments, an anti-PRLR antibody is H1H6958N₂ of WO 2015/026907 A1, comprising the CDRs of H2M6958N₂ in the same publication The expression “PRLR” includes both monomeric and multimeric PRLR molecules, such as those described in WO 2015/026907 In some embodiments, an anti-PRLR antibody comprises a heavy chain complementarity determining region (HCDR)-1 comprising SEQ ID NO: 10; an HCDR2 comprising SEQ ID NO: 11; an HCDR3 comprising SEQ ID NO: 12; a light chain complementarity determining region (LCDR)-1 comprising SEQ ID NO: 14; an LCDR2 comprising SEQ ID NO: 15; and an LCDR3 comprising SEQ ID NO: 16. In some embodiments, an anti-PRLR antibody comprises a heavy chain variable region (HCVR) comprising SEQ ID NO: 9 and a light chain variable region (LCVR) comprising SEQ ID NO: 13. In any of the foregoing embodiments, the anti-PRLR antibody can be prepared by site-directed mutagenesis to insert a glutamine residue at a site without resulting in disabled antibody function or binding. For example, in any of the foregoing embodiments, the anti-PRLR antibody can comprise an Asn297Gln (N297Q) mutation. Such antibodies having an N297Q mutation can also contain one or more additional naturally occurring glutamine residues in their variable regions, which can be accessible to transglutaminase and therefore capable of conjugation to a payload or a linker-payload (Table A). In certain embodiments, the antibody or antigen-binding fragment thereof comprises three heavy chain complementarity determining regions (HCDR1, HCDR2, and HCDR3) within a heavy chain variable region (HCVR) amino acid sequence of SEQ ID NO:9; and three light chain complementarity determining regions (LCDR1, LCDR2, and LCDR3) within a light chain variable region (LCVR) amino acid sequence of SEQ ID NO:13. In certain embodiments, the antibody or antigen-binding fragment thereof comprises an HCVR amino acid sequence of SEQ ID NO:9; and an LCVR amino acid sequence of SEQ ID NO:13. International Publication No. WO 2015/026907 A1 is hereby incorporated herein by reference in its entirety.

TABLE A
Sequences of Exemplary Antibodies H1H7814N (anti-STEAP2)
and H1H6958N (anti-PRLR)

| SEQ | Molecule/ | | |
|---|---|---|---|
| ID NO: | Antibody | Region | Sequence |
| 1 | H1H7814N | HCVR | QVQLVESGGGVVQPGRSLRLSCVASGFTISSYGMN |
| | | | WVRQAPGKGLEWVAVISYDGGNKYSVDSVKGRFT |
| | | | ISRDNSKNTLYLQMNSLRAEDSAVYYCARGRYFDL |
| | | | WGRGTLVTVSS |
| 2 | H1H7814N | HCDR1 | GFTISSYG |
| 3 | H1H7814N | HCDR2 | ISYDGGNK |
| 4 | H1H7814N | HCDR3 | ARGRYFDL |
| 5 | H1H7814N | LCVR | DIQMTQSPSTLSASVGDRVTITCRASQSISSWLAWY |
| | | | QQKPGRAPNLLISKASSLKSGVPSRFSGSGSGTEFTL |
| | | | TVSSLQPDDFATYYCQQYYSYSYTFGQGTKLEIK |
| 6 | H1H7814N | LCDR1 | QSISSW |
| 7 | H1H7814N | LCDR2 | KAS |
| 8 | H1H7814N | LCDR3 | QQYYSYSYT |
| 9 | H1H6958N2 | HCVR | QVQLVESGGGVVQPGRSLRLSCGASGFTFRNYGMQ |
| | | | WVRQGPGKGLEWVTLISFDGNDKYYADSVKGRFTI |
| | | | SRDNSKNTLFLQMNSLRTEDTAVYYCARGGDFDY |
| | | | WGQGTLVTVSS |
| 10 | H1H6958N2 | HCDR1 | GFTFRNYG |
| 11 | H1H6958N2 | HCDR2 | ISFDGNDK |
| 12 | H1H6958N2 | HCDR3 | ARGGDFDY |
| 13 | H1H6958N2 | LCVR | DIQMTQSPSSLSASVGDRVTITCRASQDIRKDLGWY |
| | | | QQKPGKAPKRLIYAASSLHSGVPSRFSGSGSGTEFTL |
| | | | TISSLQPEDFATYYCLQHNSYPMYTFGQGTKLEIK |
| 14 | H1H6958N2 | LCDR1 | QDIRKD |
| 15 | H1H6958N2 | LCDR2 | AAS |
| 16 | H1H6958N2 | LCDR3 | LQHNSYPMYT |
| 17 | hPRLR ecto- | | MHRPRRRGTRPPPLALLAALLLAARGADAQLPPGK |
| | MMH | | PEIFKCRSPNKETFTCWWRPGTDGGLPTNYSLTYHR |
| | | | EGETLMHECPDYITGGPNSCHFGKQYTSMWRTYIM |
| | | | MVNATNQMGSSFSDELYVDVTYIVQPDPPLELAVE |
| | | | VKQPEDRKPYLWIKWSPPTLIDLKTGWFTLLYEIRL |
| | | | KPEKAAEWEIHFAGQQTEFKILSLHPGQKYLVQVR |
| | | | CKPDHGYWSAWSPATFIQIPSDFTMNDEQKLISEED |
| | | | LGGEQKLISEEDLHHHHHH |

This disclosure provides antibodies or antigen-binding fragments thereof that specifically bind STEAP2, comprising an HCVR comprising an amino acid sequence selected from any of the HCVR amino acid sequences listed in Table A, or a substantially similar sequence thereof having at least 90%, at least 95%, at least 98% or at least 99% sequence identity thereto.

This disclosure also provides antibodies or antigen-binding fragments thereof that specifically bind STEAP2, comprising an LCVR comprising an amino acid sequence selected from any of the LCVR amino acid sequences listed in Table A, or a substantially similar sequence thereof having at least 90%, at least 95%, at least 98% or at least 99% sequence identity thereto.

This disclosure also provides antibodies or antigen-binding fragments thereof that specifically bind STEAP2, comprising an HCVR and an LCVR amino acid sequence pair (HCVR/LCVR) comprising any of the HCVR amino acid sequences listed in Table A paired with any of the LCVR amino acid sequences listed in Table A. According to certain embodiments, this disclosure provides antibodies, or antigen-binding fragments thereof, comprising an HCVR/LCVR amino acid sequence pair contained within any of the exemplary anti-STEAP2 antibodies listed in Table A. In certain embodiments, the HCVR/LCVR amino acid sequence pair is selected from the group consisting of: 250/258; as described in International Publication No. WO 2018/058001 A1, the contents of which are incorporated herein by reference in its entirety.

This disclosure also provides antibodies or antigen-binding fragments thereof that specifically bind STEAP2, comprising a heavy chain CDR1 (HCDR1) comprising an amino acid sequence selected from any of the HCDR1 amino acid sequences listed in Table A or a substantially similar sequence thereof having at least 90%, at least 95%, at least 98% or at least 99% sequence identity.

This disclosure also provides antibodies or antigen-binding fragments thereof that specifically bind STEAP2, comprising a heavy chain CDR2 (HCDR2) comprising an amino acid sequence selected from any of the HCDR2 amino acid sequences listed in Table A or a substantially similar sequence thereof having at least 90%, at least 95%, at least 98% or at least 99% sequence identity.

This disclosure also provides antibodies or antigen-binding fragments thereof that specifically bind STEAP2, comprising a heavy chain CDR3 (HCDR3) comprising an amino acid sequence selected from any of the HCDR3 amino acid sequences listed in Table A or a substantially similar sequence thereof having at least 90%, at least 95%, at least 98% or at least 99% sequence identity.

This disclosure also provides antibodies or antigen-binding fragments thereof that specifically bind STEAP2, comprising a light chain CDR1 (LCDR1) comprising an amino acid sequence selected from any of the LCDR1 amino acid sequences listed in Table A or a substantially similar sequence thereof having at least 90%, at least 95%, at least 98% or at least 99% sequence identity.

This disclosure also provides antibodies or antigen-binding fragments thereof that specifically bind STEAP2, comprising a light chain CDR2 (LCDR2) comprising an amino acid sequence selected from any of the LCDR2 amino acid sequences listed in Table A or a substantially similar sequence thereof having at least 90%, at least 95%, at least 98% or at least 99% sequence identity.

This disclosure also provides antibodies or antigen-binding fragments thereof that specifically bind STEAP2, comprising a light chain CDR3 (LCDR3) comprising an amino acid sequence selected from any of the LCDR3 amino acid sequences listed in Table A or a substantially similar sequence thereof having at least 90%, at least 95%, at least 98% or at least 99% sequence identity.

This disclosure also provides antibodies or antigen-binding fragments thereof that specifically bind STEAP2, comprising an HCDR3 and an LCDR3 amino acid sequence pair (HCDR3/LCDR3) comprising any of the HCDR3 amino acid sequences listed in Table A paired with any of the LCDR3 amino acid sequences listed in Table A. According to certain embodiments, this disclosure provides antibodies, or antigen-binding fragments thereof, comprising an HCDR3/LCDR3 amino acid sequence pair contained within any of the exemplary anti-STEAP2 antibodies listed in Table A. In certain embodiments, the HCDR3/LCDR3 amino acid sequence pair is selected from the group consisting of: 256/254; as described in International Publication No. WO 2018/058001 A1, the contents of which are incorporated herein by reference in its entirety.

This disclosure also provides antibodies or antigen-binding fragments thereof that specifically bind STEAP2, comprising a set of six CDRs (i.e., HCDR1-HCDR2-HCDR3-LCDR1-LCDR2-LCDR3) contained within any of the exemplary anti-STEAP2 antibodies listed in Table A. In certain embodiments, the HCDR1-HCDR2-HCDR3-LCDR1-LCDR2-LCDR3 amino acid sequence set is selected from the group consisting of: 252-254-256-260-262-264; as described in International Publication No. WO 2018/058001 A1, the contents of which are incorporated herein by reference in its entirety.

In a related embodiment, this disclosure provides antibodies, or antigen-binding fragments thereof that specifically bind STEAP2, comprising a set of six CDRs (i.e., HCDR1-HCDR2-HCDR3-LCDR1-LCDR2-LCDR3) contained within an HCVR/LCVR amino acid sequence pair as defined by any of the exemplary anti-STEAP2 antibodies listed in Table A. For example, this disclosure includes antibodies or antigen-binding fragments thereof that specifically bind STEAP2, comprising the HCDR1-HCDR2-HCDR3-LCDR1-LCDR2-LCDR3 amino acid sequence set contained within an HCVR/LCVR amino acid sequence pair selected from the group consisting of: 250/258; as described in International Publication No. WO 2018/058001 A1, the contents of which are incorporated herein by reference in its entirety. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

This disclosure provides antibodies or antigen-binding fragments thereof that specifically bind PRLR, comprising an HCVR comprising an amino acid sequence selected from any of the HCVR amino acid sequences listed in Table A, or a substantially similar sequence thereof having at least 90%, at least 95%, at least 98% or at least 99% sequence identity thereto.

This disclosure also provides antibodies or antigen-binding fragments thereof that specifically bind PRLR, comprising an LCVR comprising an amino acid sequence selected from any of the LCVR amino acid sequences listed in Table A, or a substantially similar sequence thereof having at least 90%, at least 95%, at least 98% or at least 99% sequence identity thereto.

This disclosure also provides antibodies or antigen-binding fragments thereof that specifically bind PRLR, comprising an HCVR and an LCVR amino acid sequence pair (HCVR/LCVR) comprising any of the HCVR amino acid sequences listed in Table A paired with any of the LCVR amino acid sequences listed in Table A. According to certain embodiments, this disclosure provides antibodies, or antigen-binding fragments thereof, comprising an HCVR/LCVR amino acid sequence pair contained within any of the exemplary anti-PRLR antibodies listed in Table A. In certain embodiments, the HCVR/LCVR amino acid sequence pair is selected from the group consisting of: 18/26; 66/74; 274/282; 290/298; and 370/378; as described in International Publication No. WO 2015/026907 A1, the contents of which are incorporated herein by reference in its entirety.

This disclosure also provides antibodies or antigen-binding fragments thereof that specifically bind PRLR, comprising a heavy chain CDR1 (HCDR1) comprising an amino acid sequence selected from any of the HCDR1 amino acid sequences listed in Table A or a substantially similar sequence thereof having at least 90%, at least 95%, at least 98% or at least 99% sequence identity.

This disclosure also provides antibodies or antigen-binding fragments thereof that specifically bind PRLR, comprising a heavy chain CDR2 (HCDR2) comprising an amino acid sequence selected from any of the HCDR2 amino acid sequences listed in Table A or a substantially similar sequence thereof having at least 90%, at least 95%, at least 98% or at least 99% sequence identity.

This disclosure also provides antibodies or antigen-binding fragments thereof that specifically bind PRLR, comprising a heavy chain CDR3 (HCDR3) comprising an amino acid sequence selected from any of the HCDR3 amino acid sequences listed in Table A or a substantially similar sequence thereof having at least 90%, at least 95%, at least 98% or at least 99% sequence identity.

This disclosure also provides antibodies or antigen-binding fragments thereof that specifically bind PRLR, comprising a light chain CDR1 (LCDR1) comprising an amino acid sequence selected from any of the LCDR1 amino acid sequences listed in Table A or a substantially similar sequence thereof having at least 90%, at least 95%, at least 98% or at least 99% sequence identity.

This disclosure also provides antibodies or antigen-binding fragments thereof that specifically bind PRLR, comprising a light chain CDR2 (LCDR2) comprising an amino acid sequence selected from any of the LCDR2 amino acid sequences listed in Table A or a substantially similar sequence thereof having at least 90%, at least 95%, at least 98% or at least 99% sequence identity.

This disclosure also provides antibodies or antigen-binding fragments thereof that specifically bind PRLR, comprising a light chain CDR3 (LCDR3) comprising an amino acid sequence selected from any of the LCDR3 amino acid sequences listed in Table A or a substantially similar sequence thereof having at least 90%, at least 95%, at least 98% or at least 99% sequence identity.

This disclosure also provides antibodies or antigen-binding fragments thereof that specifically bind PRLR, comprising an HCDR3 and an LCDR3 amino acid sequence pair (HCDR3/LCDR3) comprising any of the HCDR3 amino acid sequences listed in Table A paired with any of the LCDR3 amino acid sequences listed in Table A. According to certain embodiments, this disclosure provides antibodies, or antigen-binding fragments thereof, comprising an HCDR3/LCDR3 amino acid sequence pair contained within any of the exemplary anti-PRLR antibodies listed in Table A. In certain embodiments, the HCDR3/LCDR3 amino acid sequence pair is selected from the group consisting of: 24/32; 72/80; 280/288; 296/304; and 376/384; as described in International Publication No. WO 2015/026907 A1, the contents of which are incorporated herein by reference in its entirety.

This disclosure also provides antibodies or antigen-binding fragments thereof that specifically bind PRLR, comprising a set of six CDRs (i.e., HCDR1-HCDR2-HCDR3-LCDR1-LCDR2-LCDR3) contained within any of the exemplary anti-PRLR antibodies listed in Table A. In certain embodiments, the HCDR1-HCDR2-HCDR3-LCDR1-LCDR2-LCDR3 amino acid sequence set is selected from the group consisting of: 20-22-24-28-30-32; 68-70-72-76-78-80; 276-278-280-284-286-288; 292-294-296-300-302-304; and 372-374-376-380-382-384; as described in International Publication No. WO 2015/026907 A1, the contents of which are incorporated herein by reference in its entirety.

In a related embodiment, this disclosure provides antibodies, or antigen-binding fragments thereof that specifically bind PRLR, comprising a set of six CDRs (i.e., HCDR1-HCDR2-HCDR3-LCDR1-LCDR2-LCDR3) contained within an HCVR/LCVR amino acid sequence pair as defined by any of the exemplary anti-PRLR antibodies listed in Table A. For example, this disclosure includes antibodies or antigen-binding fragments thereof that specifically bind PRLR, comprising the HCDR1-HCDR2-HCDR3-LCDR1-LCDR2-LCDR3 amino acid sequence set contained within an HCVR/LCVR amino acid sequence pair selected from the group consisting of: 18/26; 66/74; 274/282; 290/298; and 370/378; as described in International Publication No. WO 2015/026907 A1, the contents of which are incorporated herein by reference in its entirety. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In any of the compound or conjugate embodiments provided, BA is an antibody, or antigen binding fragment thereof, that binds PRLR. In any of the compound or conjugate embodiments provided, BA is an antibody or antigen-binding fragment thereof, and conjugation is through at least one Q295 residue. In any of the compound or conjugate embodiments provided, BA is an antibody or antigen-binding fragment thereof, and conjugation is through two Q295 residues. In any of the compound or conjugate embodiments provided, BA is a N297Q antibody or antigen-binding fragment thereof. In any of the compound or conjugate embodiments provided, BA is a N297Q antibody or antigen-binding fragment thereof, and conjugation is through at least one Q295 and at least one Q297 residue. In any of the compound or conjugate embodiments provided, BA is a N297Q antibody or antigen-binding fragment thereof, and conjugation is through two Q295 residues and two Q297 residues. In particular embodiments, numbering is according to the EU numbering system.

In any of the embodiments above, BA is an anti-MSR1 antibody. In certain embodiments, BA is the anti-MSR1 antibody H1H21234N. In certain embodiments, BA is the anti-MSR1 antibody H1H21234N N297Q. In certain embodiments, BA is an anti-MSR1 antibody comprising an HCVR according to SEQ ID NO:19 and an LCVR according to SEQ ID NO: 27. In certain embodiments, BA is an anti-MSR1 antibody comprising one, two, three, four, five, or six of HCDR1, HCDR2, HCDR3, LCDR1, LCDR2, and LCDR3 according to SEQ ID NOS: 21, 23, 25, 29, 31, and 33, respectively. In certain embodiments, the HCVR is encoded by SEQ ID NO:18. In certain embodiments, the LCVR is encoded by SEQ ID NO: 26. In certain embodiments, one, two, three, four, five, or six of HCDR1, HCDR2, HCDR3, LCDR1, LCDR2, and LCDR3 are encoded by the polynucleotide sequences SEQ ID NOS: 20, 22, 24, 28, 30, and 32, respectively. N297Q indicates that one or more residues 297 are mutated from asparagine (N) to glutamine (Q). In certain embodiments, each residue 297 is mutated to Q. In preferred embodiments, numbering is according to the EU numbering system. In certain embodiments of this paragraph, the drug:antibody ratio (DAR) is from 1 to 4. In certain embodiments, DAR is 1, 2, 3, or 4. In certain embodiments, DAR is 2. In certain embodiments, DAR is 4.

This disclosure provides antibodies or antigen-binding fragments thereof that specifically bind MSR1, comprising an HCVR comprising an amino acid sequence selected from any of the HCVR amino acid sequences listed in Table B, or a substantially similar sequence thereof having at least 90%, at least 95%, at least 98% or at least 99% sequence identity thereto.

This disclosure also provides antibodies or antigen-binding fragments thereof that specifically bind MSR1, comprising an LCVR comprising an amino acid sequence selected from any of the LCVR amino acid sequences listed in Table B, or a substantially similar sequence thereof having at least 90%, at least 95%, at least 98% or at least 99% sequence identity thereto.

This disclosure also provides antibodies or antigen-binding fragments thereof that specifically bind MSR1, comprising an HCVR and an LCVR amino acid sequence pair (HCVR/LCVR) comprising any of the HCVR amino acid sequences listed in Table B paired with any of the LCVR amino acid sequences listed in Table B. According to certain embodiments, this disclosure provides antibodies, or antigen-binding fragments thereof, comprising an HCVR/LCVR amino acid sequence pair contained within any of the exemplary anti-MSR1 antibodies listed in Table B. In certain embodiments, the HCVR/LCVR amino acid sequence pair is selected from the group consisting of: 2/10, 23/42, 50/58; 90/98, and 282/290; as described in International Publication No. WO 2019/217597 A1, the contents of which are incorporated herein by reference in its entirety.

This disclosure also provides antibodies or antigen-binding fragments thereof that specifically bind MSR1, comprising a heavy chain CDR1 (HCDR1) comprising an amino acid sequence selected from any of the HCDR1 amino acid sequences listed in Table B or a substantially similar sequence thereof having at least 90%, at least 95%, at least 98% or at least 99% sequence identity.

This disclosure also provides antibodies or antigen-binding fragments thereof that specifically bind MSR1, comprising a heavy chain CDR2 (HCDR2) comprising an amino acid sequence selected from any of the HCDR2 amino acid sequences listed in Table B or a substantially similar sequence thereof having at least 90%, at least 95%, at least 98% or at least 99% sequence identity.

This disclosure also provides antibodies or antigen-binding fragments thereof that specifically bind MSR1, comprising a heavy chain CDR3 (HCDR3) comprising an amino acid sequence selected from any of the HCDR3 amino acid sequences listed in Table B or a substantially similar sequence thereof having at least 90%, at least 95%, at least 98% or at least 99% sequence identity.

This disclosure also provides antibodies or antigen-binding fragments thereof that specifically bind MSR1, comprising a light chain CDR1 (LCDR1) comprising an amino acid sequence selected from any of the LCDR1 amino acid sequences listed in Table B or a substantially similar sequence thereof having at least 90%, at least 95%, at least 98% or at least 99% sequence identity.

This disclosure also provides antibodies or antigen-binding fragments thereof that specifically bind MSR1, comprising a light chain CDR2 (LCDR2) comprising an amino acid sequence selected from any of the LCDR2 amino acid sequences listed in Table B or a substantially similar sequence thereof having at least 90%, at least 95%, at least 98% or at least 99% sequence identity.

This disclosure also provides antibodies or antigen-binding fragments thereof that specifically bind MSR1, comprising a light chain CDR3 (LCDR3) comprising an amino acid sequence selected from any of the LCDR3 amino acid sequences listed in Table B or a substantially similar sequence thereof having at least 90%, at least 95%, at least 98% or at least 99% sequence identity.

This disclosure also provides antibodies or antigen-binding fragments thereof that specifically bind MSR1, comprising an HCDR3 and an LCDR3 amino acid sequence pair (HCDR3/LCDR3) comprising any of the HCDR3 amino acid sequences listed in Table B paired with any of the LCDR3 amino acid sequences listed in Table B. According to certain embodiments, this disclosure provides antibodies, or antigen-binding fragments thereof, comprising an HCDR3/LCDR3 amino acid sequence pair contained within any of the exemplary anti-MSR1 antibodies listed in Table B. In certain embodiments, the HCDR3/LCDR3 amino acid sequence pair is selected from the group consisting of: 8/16, 40/48, 56/64; 96/104, and 288/296; as described in International Publication No. WO 2019/217591 A1, the contents of which are incorporated herein by reference in its entirety.

This disclosure also provides antibodies or antigen-binding fragments thereof that specifically bind MSR1, comprising a set of six CDRs (i.e., HCDR1-HCDR2-HCDR3-LCDR1-LCDR2-LCDR3) contained within any of the exemplary anti-MSR1 antibodies listed in Table B. In certain embodiments, the HCDR1-HCDR2-HCDR3-LCDR1-LCDR2-LCDR3 amino acid sequence set is selected from the group consisting of: 4-6-8-12-14-16; 36-38-40-44-46-48; 52-54-56-60-62-64; 92-94-96-100-102-104, and 284-286-288-292-294-2%; as described in International Publication No. WO 2019/217591 A1, the contents of which are incorporated herein by reference in its entirety.

In a related embodiment, this disclosure provides antibodies, or antigen-binding fragments thereof that specifically bind MSR1, comprising a set of six CDRs (i.e., HCDR1-HCDR2-HCDR3-LCDR1-LCDR2-LCDR3) contained within an HCVR/LCVR amino acid sequence pair as defined by any of the exemplary anti-MSR1 antibodies listed in Table B. For example, this disclosure includes antibodies or antigen-binding fragments thereof that specifically bind MSR1, comprising the HCDR1-HCDR2-HCDR3-LCDR1-LCDR2-LCDR3 amino acid sequence set contained within an HCVR/LCVR amino acid sequence pair selected from the group consisting of: 2/10, 23/42, 50/58, 90/98, and 282/290; as described in International Publication No. WO 2019/217591 A1, the contents of which are incorporated herein by reference in its entirety. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

TABLE B
Sequences of Exemplary anti-MSR1 Antibodies

| SEQ | | | |
|---|---|---|---|
| ID | Molecule/ | | |
| NO: | Antibody | Region | Sequence |
| 18 | H1H21234N | HCVR | caggtgcagc tgcaggagtc gggcccagga |
| | | | ctggtgaagc cttcggagac cctgtccctc |
| | | | acctgcactg tcactggtgg ctccatcagt |
| | | | aggaactact ggagttggat ccggcagccc |
| | | | ccagggaagg gactggaatg gattggatat |
| | | | atctattaca gtgggagtat cgactacaat |
| | | | ccctccctca agagtcgagt caccatatca |
| | | | gtagacacgt ccaagaacca gttctccctg |
| | | | aagctgagtt ctatgaccgc tgcggacacg |
| | | | gccgtatact actgtgcgag agatcggtgg |
| | | | aactggaaat acggtatgga cgtctggggc |
| | | | caagggacca cggtcatcgt ctcgtca |
| 19 | H1H21234N | HCVR | Gln Val Gln Leu Gln Glu Ser Gly Pro Gly |
| | | | Leu Val Lys Pro Ser Glu Thr Leu Ser Leu |
| | | | Thr Cys Thr Val Thr Gly Gly Ser Ile Ser |
| | | | Arg Asn Tyr Trp Ser Trp Ile Arg Gln Pro |
| | | | Pro Gly Lys Gly Leu Glu Trp Ile Gly Tyr |
| | | | Ile Tyr Tyr Ser Gly Ser Ile Asp Tyr Asn |
| | | | Pro Ser Leu Lys Ser Arg Val Thr Ile Ser |
| | | | Val Asp Thr Ser Lys Asn Gln Phe Ser Leu |
| | | | Lys Leu Ser Ser Met Thr Ala Ala Asp Thr |
| | | | Ala Val Tyr Tyr Cys Ala Arg Asp Arg Trp |
| | | | Asn Trp Lys Tyr Gly Met Asp Val Trp Gly |
| | | | Gln Gly Thr Thr Val Ile Val Ser Ser |
| 20 | H1H21234N | HCDR1 | ggtggctcca tcagtaggaa ctac |
| 21 | H1H21234N | HCDR1 | Gly Gly Ser Ile Ser Arg Asn Tyr |
| 22 | H1H21234N | HCDR2 | atctattaca gtgggagtat c |
| 23 | H1H21234N | HCDR2 | Ile Tyr Tyr Ser Gly Ser Ile |
| 24 | H1H21234N | HCDR3 | gcgagagatc ggtggaactg gaaatacggt |
| | | | atggacgtc |
| 25 | H1H21234N | HCDR3 | Ala Arg Asp Arg Trp Asn Trp Lys Tyr Gly |
| | | | Met Asp Val |
| 26 | H1H21234N | LCVR | gaaattgtgt tgacgcagtc tccaggcacc |
| | | | ctgtctttgt ctccagggga aagagccacc |
| | | | ctctcctgca gggccagtca gactgttaga |
| | | | aacaactact tagcctggta ccaccagaaa |
| | | | cctggccagg ctcccaggct cctcatctat |
| | | | ggtgcatcca gcagggccac |
| | | | tggcatccca gacaggttca gtggcagtgg |
| | | | gtctgggaca gacttcactc tcaccatcag |
| | | | cagactggag cctgaagatt ttacagtgta |
| | | | ttactgtcac cagtatggta actcaccttg |
| | | | gacgttcggc caagggacca aaatggaaat |
| | | | caaacga |
| 27 | H1H21234N | LCVR | Glu Ile Val Leu Thr Gln Ser Pro Gly Thr |
| | | | Leu Ser Leu Ser Pro Gly Glu Arg Ala Thr |
| | | | Leu Ser Cys Arg Ala Ser Gln Thr Val Arg |
| | | | Asn Asn Tyr Leu Ala Trp Tyr His Gln Lys |
| | | | Pro Gly Gln Ala Pro Arg Leu Leu Ile Tyr |
| | | | Gly Ala Ser Ser Arg Ala Thr Gly Ile Pro |
| | | | Asp Arg Phe Ser Gly Ser Gly Ser Gly Thr |
| | | | Asp Phe Thr Leu Thr Ile Ser Arg Leu Glu |
| | | | Pro Glu Asp Phe Thr Val Tyr Tyr Cys His |
| | | | Gln Tyr Gly Asn Ser Pro Trp Thr Phe Gly |
| | | | Gln Gly Thr Lys Met Glu Ile Lys Arg |
| 28 | H1H21234N | LCDR1 | cagactgtta gaaacaacta c |
| 29 | H1H21234N | LCDR1 | Gln Thr Val Arg Asn Asn Tyr |
| 30 | H1H21234N | LCDR2 | ggtgcatcc |
| 31 | H1H21234N | LCDR2 | Gly Ala Ser |
| 32 | H1H21234N | LCDR3 | caccagtatg gtaactcacc ttggacg |
| 33 | H1H21234N | LCDR3 | His Gln Tyr Gly Asn Ser Pro Trp Thr |

The binding agent linkers can be bonded to the binding agent, e.g., antibody or antigen-binding molecule, through an attachment at a particular amino acid within the antibody or antigen-binding molecule. Exemplary amino acid attachments that can be used in the context of this embodiment of the disclosure include, e.g., lysine (see, e.g., U.S. Pat. No. 5,208,020; US 2010/0129314; Hollander et al., Bioconjugate Chem., 2008, 19:358-361; WO 2005/089808; U.S. Pat. No. 5,714,586; US 2013/0101546; and US 2012/0585592), cysteine (see, e.g., US 2007/0258987; WO 2013/055993; WO 2013/055990; WO 2013/053873; WO 2013/053872; WO 2011/130598; US 2013/0101546; and U.S. Pat. No. 7,750,116), selenocysteine (see, e.g., WO 2008/122039; and Hofer et al., Proc. Natl. Acad. Sci., USA, 2008, 105:12451-12456), formyl glycine (see, e.g., Carrico et al., Nat. Chem. Biol., 2007, 3:321-322; Agarwal et al., Proc. Natl. Acad Sci., USA, 2013, 110:46-51, and Rabuka et al., Nat. Protocols, 2012, 10:1052-1067), non-natural amino acids (see, e.g., WO 2013/068874, and WO 2012/166559), and acidic amino acids (see, e.g., WO 2012/05982). Linkers can also be conjugated to an antigen-binding protein via attachment to carbohydrates (see, e.g., US 2008/0305497, WO 2014/065661, and Ryan et al., Food & Agriculture Immunol., 2001, 13:127-130).

In some examples, the binding agent is an antibody or antigen binding molecule, and the antibody is bonded to the linker through a lysine residue. In some embodiments, the antibody or antigen binding molecule is bonded to the linker through a cysteine residue.

Linkers can also be conjugated to one or more glutamine residues via transglutaminase-based chemo-enzymatic conjugation (see, e.g., Dennler et al., Bioconjugate Chem. 2014, 25, 569-578). For example, in the presence of transglutaminase, one or more glutamine residues of an antibody can be coupled to a primary amine compound. Primary amine compounds include, e.g., payloads or linker-payloads, which directly provide antibody drug conjugates via transglutaminase-mediated coupling. Primary amine compounds also include linkers and spacers that are functionalized with reactive groups that can be subsequently reacted with further compounds towards the synthesis of antibody drug conjugates. Antibodies comprising glutamine residues can be isolated from natural sources or engineered to comprise one or more glutamine residues. Techniques for engineering glutamine residues into an antibody polypeptide chain (glutaminyl-modified antibodies or antigen binding molecules) are within the skill of the practitioners in the art. In certain embodiments, the antibody is aglycosylated.

In certain embodiments, the antibody or a glutaminyl-modified antibody or antigen binding molecule comprises at least one glutamine residue in at least one polypeptide chain sequence. In certain embodiments, the antibody or a glutaminyl-modified antibody or antigen binding molecule comprises two heavy chain polypeptides, each with one Gln295 or Q295 residue. In further embodiments, the antibody or a glutaminyl-modified antibody or antigen binding molecule comprises one or more glutamine residues at a site other than a heavy chain 295. Included herein are antibodies of this section bearing N297Q mutation(s) described herein.

### Primary Amine Compounds

In certain embodiments, primary amine compounds useful for the transglutaminase mediated coupling of an antibody (or antigen binding compound) comprising a glutamine can be any primary amine compound deemed useful by the practitioner of ordinary skill. Generally, the primary amine compound has the formula H₂N—R, where R can be any group compatible with the antibody and reaction conditions. In certain embodiments, R is alkyl, substituted alkyl, heteroalkyl, or substituted heteroalkyl.

In some embodiments, the primary amine compound comprises a reactive group or protected reactive group. Useful reactive groups include azides, alkynes, cycloalkynes, thiols, alcohols, ketones, aldehydes, carboxylic acids, esters, amides, hydrazides, anilines, and amines. In certain embodiments, the reactive group is selected from the group consisting of azide, alkyne, sulfhydryl, cycloalkyne, aldehyde, and carboxyl.

In certain embodiments, the primary amine compound is according to the formula H2N-LL-X, where LL is a divalent spacer and X is a reactive group or protected reactive group. In particular embodiments, LL is a divalent polyethylene glycol (PEG) group. In certain embodiments, X is selected from the group consisting of —SH, —N₃, alkyne, aldehyde, and tetrazole. In particular embodiments, X is —N₃.

In certain embodiments, the primary amine compound is according to one of the following formulae:
H₂N—(CH₂)ₙ—X;
H₂N—(CH₂CH₂O)ₙ—(CH₂)ₚ—X;
H₂N—(CH₂)ₙ—N(H)C(O)—(CH₂)ₘ—X;
H₂N—(CH₂CH₂O)ₙ—N(H)C(O)—(CH₂CH₂O)ₘ—(CH₂)ₚ—X;
H₂N—(CH₂)ₙ—C(O)N(H)—(CH₂)ₘ—X;
H₂N—(CH₂CH₂O)ₙ—C(O)N(H)—(CH₂CH₂O)ₘ—(CH₂)ₚ—X;
H₂N—(CH₂)ₙ—N(H)C(O)—(CH₂CH₂O)ₘ—(CH₂)ₚ—X;
H₂N—(CH₂CH₂O)ₙ—N(H)C(O)—(CH₂)ₘ—X;
H₂N—(CH₂)ₙ—C(O)N(H)—(CH₂CH₂O)ₘ—(CH₂)ₚ—X; and
H₂N—(CH₂CH₂O)ₙ—C(O)N(H)—(CH₂)ₘ—X;
   where n is an integer selected from 1 to 12;
   m is an integer selected from 0 to 12;
   p is an integer selected from 0 to 2;
   and X is selected from the group consisting of —SH, —N₃, —C≡CH, —C(O)H, tetrazole, and any of

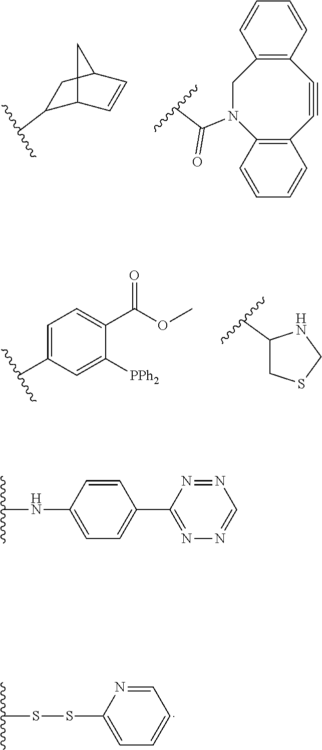

In the above, any of the alkyl or alkylene (i.e., —CH₂—) groups can optionally be substituted, for example with C₁₋₈ alkyl, methylformyl, or —SO₃H. In certain embodiments, the alkyl groups are unsubstituted.

In certain embodiments, the primary amine compound is selected from the group consisting of:

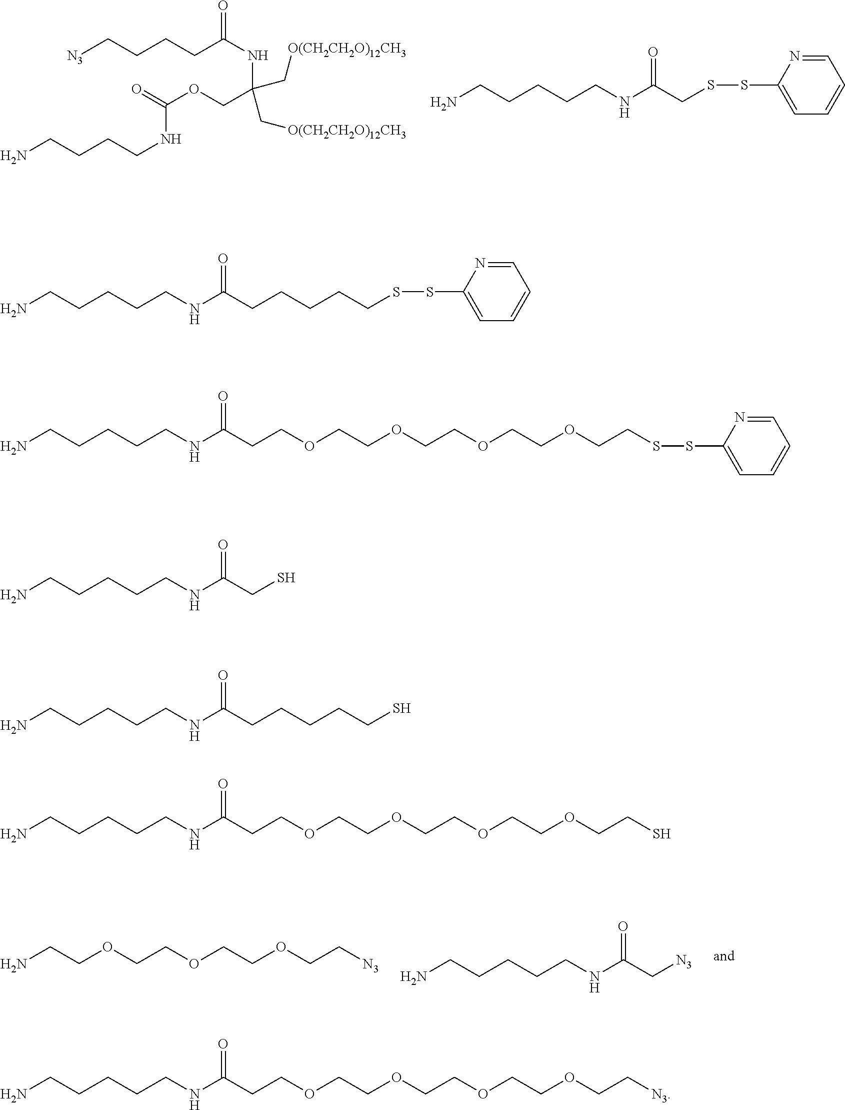

In particular embodiments, the primary amine compound is

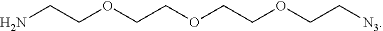 [SMILES: [N-]=[N+]=NCCOCCOCCOCCN]

Exemplary conditions for the above reactions are provided in the Examples below.

### Linkers

In certain embodiments, the linker L portion of the conjugates described herein is a moiety, for instance a divalent moiety, that covalently links a binding agent to a payload compound described herein. In other instances, the linker L is a trivalent or multivalent moiety that covalently links a binding agent to a payload compound described herein. Suitable linkers may be found, for example, in Antibody-Drug Conjugates and Immunotoxins; Phillips, G. L., Ed.; Springer Verlag: New York, 2013; Antibody Drug Conjugates; Ducry, L., Ed.; Humana Press, 2013; Antibody-Drug Conjugates; Wang, J., Shen, W.-C., and Zaro, J. L., Eds.; Springer International Publishing, 2015, the contents of each incorporated herein in their entirety by reference. In certain embodiments, the linker L portion of the linker-payloads described herein is a moiety covalently linked to a payload compound described herein, capable of divalently and covalently linking a binding agent to a payload compound described herein. In other instances, the linker L portion of the linker-payloads described herein is a moiety covalently linked to a payload compound described herein, capable of covalently linking, as a trivalent or multivalent moiety, a binding agent to a payload compound described herein. Payload compounds include compounds of Formulae I, Ib, Ibb, and Ibbb above, and their residues following bonding or incorporation with linker L are linker-payload compounds. The linker-payloads can be further bonded to binding agents such as antibodies or antigen binding fragments thereof to form antibody-drug conjugates. Those of skill in the art will recognize that certain functional groups of payload moieties are convenient for linking to linkers and/or binding agents. For example, in certain embodiments, the linker is absent and payloads are directly bonded to binding agents. In certain embodiments, prodrugs or payloads include hydroxyl, amine, or thiol functionality capable of bonding with peptide residues within binding agents.

In certain embodiments, the linkers are stable in physiological conditions. In certain embodiments, the linkers are cleavable, for instance, able to release at least the payload portion in the presence of an enzyme or at a particular pH range or value. In some embodiments, a linker comprises an enzyme-cleavable moiety. Illustrative enzyme-cleavable moieties include, but are not limited to, peptide bonds, ester linkages, hydrazones, and disulfide linkages. In some embodiments, the linker comprises a cathepsin-cleavable linker. In some embodiments, the linker comprises a moiety that is stable at certain pHs and cleavable to release the payload portion at other pHs. For instance, in certain embodiments, the linker is stable at physiological pH and capable of releasing the payload portion at a local pH in the vicinity of a target.

In some embodiments, the linker comprises a non-cleavable moiety. In some embodiments, the non-cleavable linker is derived from maleimide. In some embodiments, the non-cleavable linkers are derived from an ester. In some embodiments, the non-cleavable linker is derived from an N-hydroxysuccinimide ester. In some embodiments, the non-cleavable linker is derived from

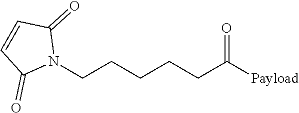

or a residue thereof. In some embodiments, the non-cleavable linker-payload residue is

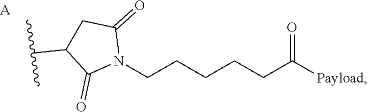

or a regioisomer thereof. In some embodiments, the non-cleavable linker is derived from

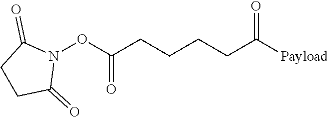

or a residue thereof. In some embodiments, the non-cleavable linker-payload residue is

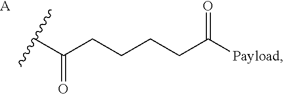

or a regioisomer thereof. In one embodiment, the linker is maleimide cyclohexane carboxylate or 4-(N-maleimidomethyl)cyclohexanecarboxylic acid (MCC), where the payload can be added to either end of the MCC linker. In another embodiment, the linker is

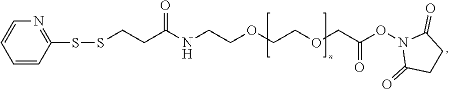

where the payload can be added to either end of this linker. In certain embodiments, the linker is a self-stabilizing maleimide. In one exemplary embodiment, the self-stabilizing maleimide linker is

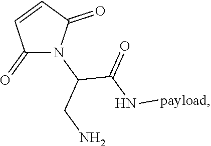

where the bond from the amide nitrogen to the payload can be a direct bond to the payload; or the bond from the amide nitrogen to the payload, as shown, contemplates the remainder of the linker. In another exemplary embodiment, the self-stabilizing linker manifests as

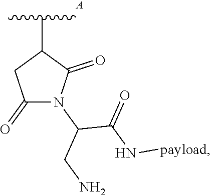

where the bond from the amide nitrogen to the payload can be a direct bond to the payload; or the bond from the amide nitrogen to the payload, as shown, contemplates the remainder of the linker. Without being bound by any particular theory, the self-stabilizing linker includes moieties that stabilize the bond from the self-stabilizing linker to a binding agent. For example, in some embodiments, when the bond from a binding agent to a self-stabilizing linker is a carbon-sulfur bond (e.g., following a Michael addition of a binding agent cysteine to the self-stabilizing maleimide linker), the self-stabilizing linker mitigates retro-Michael additions. More specifically, in the self-stabilizing maleimide (or succinimide) linkers shown, the aminomethyl functionality facilitates rapid hydrolysis of the succinimide Michael addition product to provide

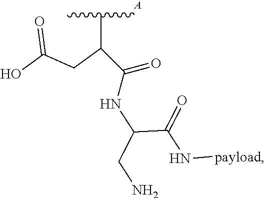

where the bond from the amide nitrogen to the payload can be a direct bond to the payload; or the bond from the amide nitrogen to the payload, as shown, contemplates the remainder of the linker; thus, decreasing susceptibility to retro-Michael additions. Moieties other than aminomethyl within self-stabilizing maleimide linkers that stabilize conjugates will be appreciated by those of skill in the art. In the structures,

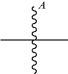

indicates a bond to a binding agent. In the structures, in some examples,

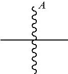

indicates a click chemistry residue which results from the reaction of, for example, a binding agent having an azide or alkyne functionality and a linker-payload having a complementary alkyne or azide functionality. In the structures, in other examples,

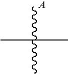

indicates a divalent sulfide which results from the reaction of, for example, one or more binding agent cysteines with one or more linkers or linker-payloads having maleimide functionality via Michael addition reactions. In the structures, in other examples,

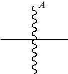

indicates an amide bond which results from the reaction of, for example, one or more binding agent lysines with one or more linkers or linker-payloads having activated or unactivated carboxyl functionality, as would be appreciated by a person of skill in the art. In one embodiment,

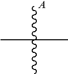

indicates an amide bond which results from the reaction of, for example, one or more binding agent lysines with one or more linkers or linker-payloads having activated carboxyl functionality, as would be appreciated by a person of skill in the art.

In some embodiments, suitable linkers include, but are not limited to, those that are chemically bonded to two cysteine residues of a single binding agent, e.g., antibody. Such linkers can serve to mimic the antibody's disulfide bonds that are disrupted as a result of the conjugation process.

In some embodiments, the linker comprises one or more amino acids. Suitable amino acids include natural, non-natural, standard, non-standard, proteinogenic, non-proteinogenic, and L- or D- α-amino acids. In some embodiments, the linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or any combination thereof (e.g., dipeptides, tripeptides, oligopeptides, polypeptides, and the like). In certain embodiments, one or more side chains of the amino acids are linked to a side chain group, described below. In some embodiments, the linker is a peptide comprising or consisting of the amino acids valine and citrulline (e.g., divalent -Val-Cit- or divalent -VCit-). In some embodiments, the linker is a peptide comprising or consisting of the amino acids alanine and alanine, or divalent -AA-. In some embodiments, the linker is a peptide comprising or consisting of the amino acids glutamic acid and alanine, or -EA-. In some embodiments, the linker is a peptide comprising or consisting of the amino acids glutamic acid and glycine, or -EG-. In some embodiments, the linker is a peptide comprising or consisting of the amino acids glycine and glycine, or -GG-. In some embodiments, the linker is a peptide comprising or consisting of the amino acids glutamine, valine, and citrulline, or -Q-V-Cit- or -QVCit-. In some embodiments, the linker is a peptide comprising or consisting of the amino acids glutamic acid, valine, and citrulline, or -E-V-Cit- or -EVCit-. In some embodiments, the linker is a peptide comprising or consisting of the amino acids -GGGGS-. In some embodiments, the linker is a peptide comprising or consisting of the amino acids -GGGGG-. In some embodiments, the linker is a peptide comprising or consisting of the amino acids -GGGGK-. In some embodiments, the linker is a peptide comprising or consisting of the amino acids -GFGG-. In some embodiments, the linker is a peptide comprising or consisting of the amino acids lysine, valine, and citrulline, or -KVCit-. In some embodiments, the linker is a peptide comprising or consisting of the amino acids -KVA-. In some embodiments, the linker is a peptide comprising or consisting of the amino acids -VA-. In any of the embodiments in this paragraph, and throughout this disclosure, the standard three-letter or one-letter amino acid designations are used, as would be appreciated by a person of skill in the art. Exemplary single-letter amino acid designations include, G for glycine, K for lysine, S for serine, V for valine, A for alanine, and F for phenylalanine.

In some embodiments, the linker comprises a self-immolative group. The self-immolative group can be any such group known to those of skill. In particular embodiments, the self-immolative group is p-aminobenzyl (PAB), or a derivative thereof. Useful derivatives include p-aminobenzyloxycarbonyl (PABC). Those of skill will recognize that a self-immolative group is capable of carrying out a chemical reaction which releases the remaining atoms of a linker from a payload.

In some embodiments, the linker is:

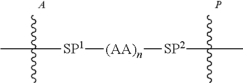

wherein:

SP¹ is a spacer;

SP² is a spacer;

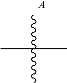

is one or more bonds to the binding agent;

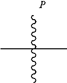

is one or more bonds to the payload;

each AA is an amino acid residue; and

n is an integer from 0 to 10.

The SP¹ spacer is a moiety that connects the (AA)ₙ moiety or residue to the binding agent (BA) or to a reactive group residue which is bonded to BA. Suitable SP¹ spacers include, but are not limited to, those comprising alkylene or polyether, or both. The ends of the spacers, for example, the portion of the spacer bonded to the BA or an AA, can be moieties derived from reactive moieties that are used for purposes of coupling the antibody or an AA to the spacer during chemical synthesis of the conjugate. In certain embodiments, n is 1, 2, 3, or 4. In particular embodiments, n is 2. In particular embodiments, n is 3. In particular embodiments, n is 4. In certain embodiments, when n is zero, then (AA)ₙ is a bond.

In some embodiments, the SP¹ spacer comprises an alkylene. In some embodiments, the SP¹ spacer comprises a C₅₋₇ alkylene. In some embodiments, the SP¹ spacer comprises a polyether. In some embodiments, the SP¹ spacer comprises a polymer of ethylene oxide such as polyethylene glycol.

In some embodiments, the SP¹ spacer is:

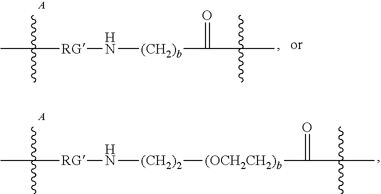

wherein:

RG′ is a reactive group residue following reaction of a reactive group RG with a binding agent;

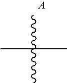

is a bond to the binding agent;

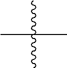

is a bond to (AA)ₙ wherein n is an integer from 0 to 10; and

b is an integer from 2 to 8.

The reactive group RG can be any reactive group known to those of skill in the art to be capable of forming one or more bonds to the binding agent. The reactive group RG is a moiety comprising a portion in its structure that is capable of reacting with the binding agent (e.g., reacting with an antibody at its cysteine or lysine residues, or at an azide moiety, for example, a PEG-N₃ functionalized antibody at one or more glutamine residues) to form a compound of Formula III. Following conjugation to the binding agent, the reactive group becomes the reactive group residue (RG′). Illustrative reactive groups include, but are not limited to, those that comprise haloacetyl, isothiocyanate, succinimide, N-hydroxysuccinimide, or maleimide portions that are capable of reacting with the binding agent.

In certain embodiments, reactive groups include, but are not limited to, alkynes. In certain embodiments, the alkynes are alkynes capable of undergoing 1,3-cycloaddition reactions with azides in the absence of copper catalysts, such as strained alkynes. Strained alkynes are suitable for strain-promoted alkyne-azide cycloadditions (SPAAC), and include cycloalkynes, for example, cyclooctynes and benzannulated alkynes. Suitable alkynes include, but are not limited to, dibenzoazacyclooctyne or

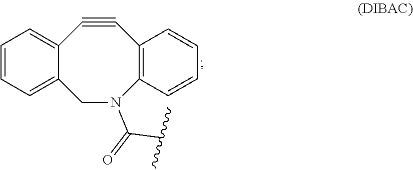 [SMILES: *C(=O)N1Cc2ccccc2C#Cc2ccccc21]

dibenzocyclooctyne or

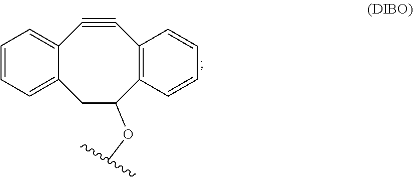 [SMILES: *OC1Cc2ccccc2C#Cc2ccccc21]

biarylazacyclooctynone or

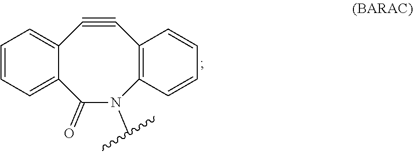 [SMILES: *N1C(=O)c2ccccc2C#Cc2ccccc21]

difluorinated cyclooctyne or

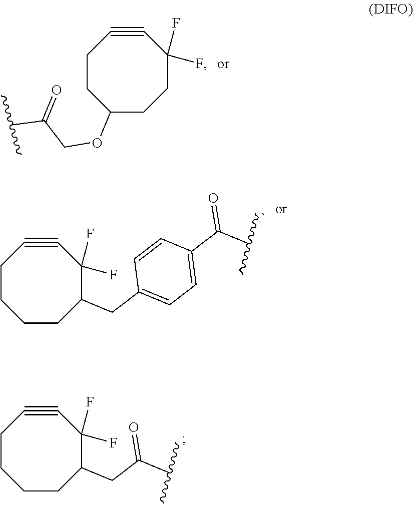 [SMILES: *C(=O)COC1CCC#CC(F)(F)CC1 ; *C(=O)c1ccc(CC2CCCCC#CC2(F)F)cc1 ; *C(=O)CC1CCCCC#CC1(F)F]

substituted, for example, fluorinated alkynes, aza-cycloalkynes, bicycle[6.1.0]nonyne or

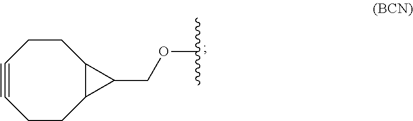 [SMILES: *OCC1C2CCC#CCCC21]

and derivatives thereof. Particularly useful alkynes include

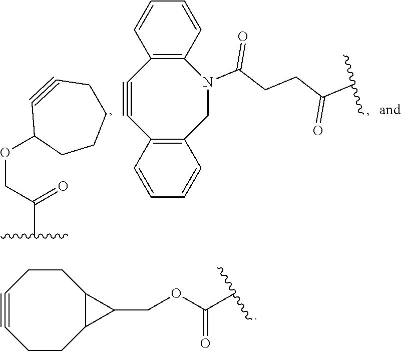

In certain embodiments, the binding agent is bonded directly to RG′. In certain embodiments, the binding agent is bonded to RG′ via a spacer, for instance SP⁴, located between

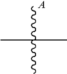

and RG′. In particular embodiments, the binding agent is bonded indirectly to RG′ via SP⁴, for example, a PEG spacer. As discussed in detail below, in certain embodiments, the binding agent is prepared by functionalizing with one or more azido groups. Each azido group is capable of reacting with RG to form RG′. In particular embodiments, the binding agent is derivatized with -PEG-N₃ linked to a glutamine residue. Exemplary —N₃ derivatized binding agents, methods for their preparation, and methods for their use in reacting with RG are provided herein. In certain embodiments, RG is an alkyne suitable for participation in 1,3-cycloadditions, and RG′ is a regioisomeric 1,2,3-triazolyl moiety formed from the reaction of RG with an azido-functionalized binding agent. By way of further example, in certain embodiments, RG′ is linked to the binding agent as shown in

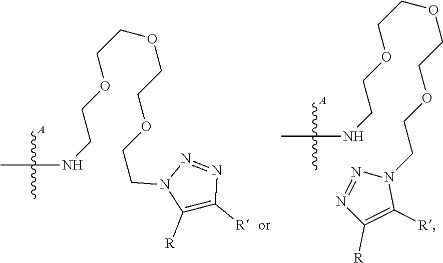

or a mixture of each regioisomer. Each R and R′ is as described or exemplified herein.

The SP² spacer, when present, is a moiety that connects the (AA)ₙ moiety to the payload. Suitable spacers include, but are not limited to, those described above as SP¹ spacers. Further suitable SP² spacers include, but are not limited to, those comprising alkylene or polyether, or both. The ends of the SP² spacers, for example, the portion of the spacer directly bonded to the payload or an AA, can be moieties derived from reactive moieties that are used for purposes of coupling the payload or AA to the SP² spacer during the chemical synthesis of the conjugate. In some examples, the ends of the SP² spacers, for example, the portion of the SP² spacer directly bonded to the payload or an AA, can be residues of reactive moieties that are used for purposes of coupling the payload or an AA to the spacer during the chemical synthesis of the conjugate.

In some embodiments, the SP² spacer, when present, is selected from the group consisting of —NH-(p-C₆H₄)—CH₂—, —NH-(p-C₆H₄)—CH₂OC(O)—, an amino acid, a dipeptide, a tripeptide, an oligopeptide, —O—, —N(H)—,

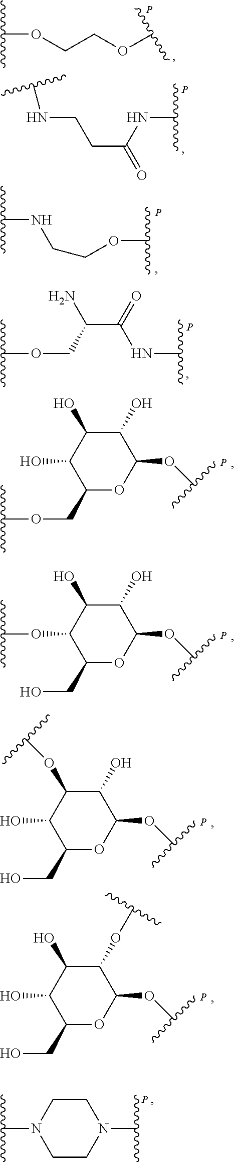

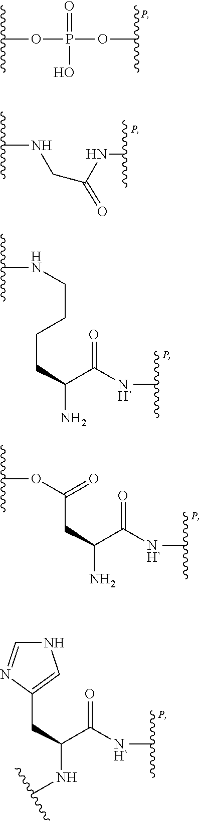

and any combinations thereof. In certain embodiments, each

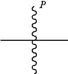

is a bond to the payload, and each

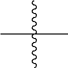

is a bond to (AA)ₙ.

In the above formulae, each (AA)ₙ is an amino acid or, optionally, a p-aminobenzyloxycarbonyl residue (PABC),

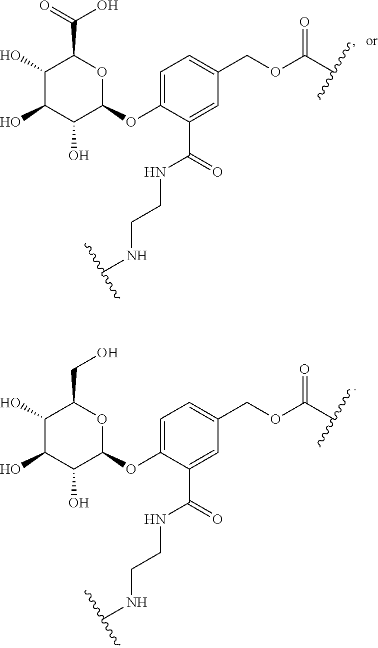 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O]

If PABC is present, in certain embodiments, then only one PABC is present. In certain embodiments, the PABC residue, if present, is bonded to a terminal AA in the (AA)ₙ group, proximal to the payload. If

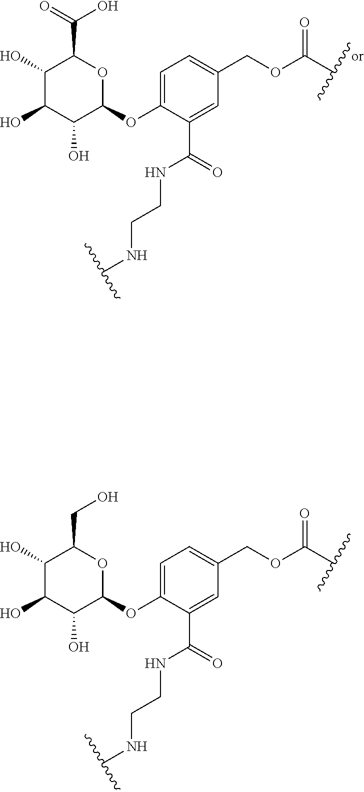 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O]

is present, then only

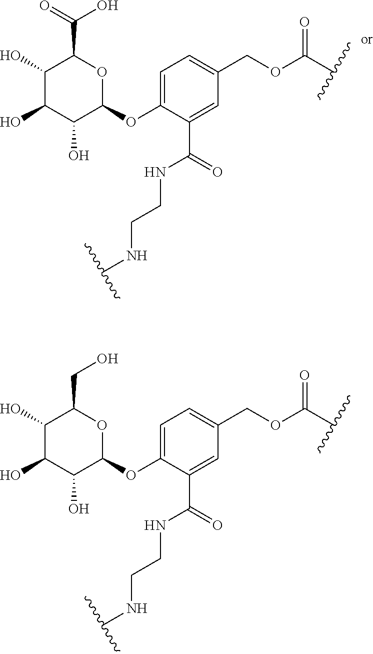 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O]

is present. In certain embodiments, the

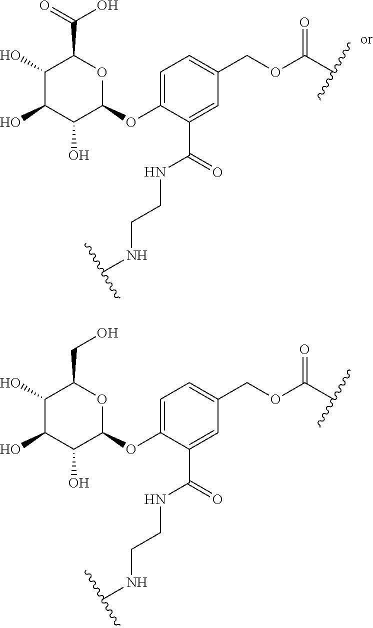 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O]

residue, if present, is bonded to the payload via the benzyloxycarbonyl moiety, and no AA is present. Suitable amino acids for each AA include natural, non-natural, standard, non-standard, proteinogenic, non-proteinogenic, and L- or D- α-amino acids. In some embodiments, the AA comprises alanine, valine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or any combinations thereof (e.g., dipeptides, tripeptides, and oligopeptides, and the like). In certain embodiments, one or more side chains of the amino acids is linked to a side chain group, described below. In some embodiments, n is two. In some embodiments, the (AA)ₙ is valine-citrulline. In some embodiments, (AA)ₙ is citrulline-valine. In some embodiments, (AA)ₙ is valine-alanine. In some embodiments, (AA)ₙ is alanine-valine. In some embodiments, (AA)ₙ is valine-glycine. In some embodiments, (AA)ₙ is glycine-valine. In some embodiments, n is three. In some embodiments, the (AA)ₙ is valine-citrulline-PABC. In some embodiments, (AA)ₙ is citrulline-valine-PABC. In some embodiments, (AA)ₙ is glutamate-valine-citrulline. In some embodiments, (AA)ₙ is glutamine-valine-citrulline. In some embodiments, (AA)ₙ is lysine-valine-alanine. In some embodiments, (AA)ₙ is lysine-valine-citrulline. In some embodiments, n is four. In some embodiments, (AA)ₙ is glutamate-valine-citrulline-PABC. In some embodiments, (AA)ₙ is glutamine-valine-citrulline-PABC. Those of skill will recognize PABC as a residue of p-aminobenzyloxycarbonyl with the following structure:

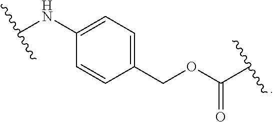 [SMILES: *Nc1ccc(COC(*)=O)cc1]

The PABC residue has been shown to facilitate cleavage of certain linkers in vitro and in vivo. Those of skill will recognize PAB as a divalent residue of p-aminobenzyl or —NH-(p-C₆H₄)—CH₂—.

In some embodiments, the linker is:

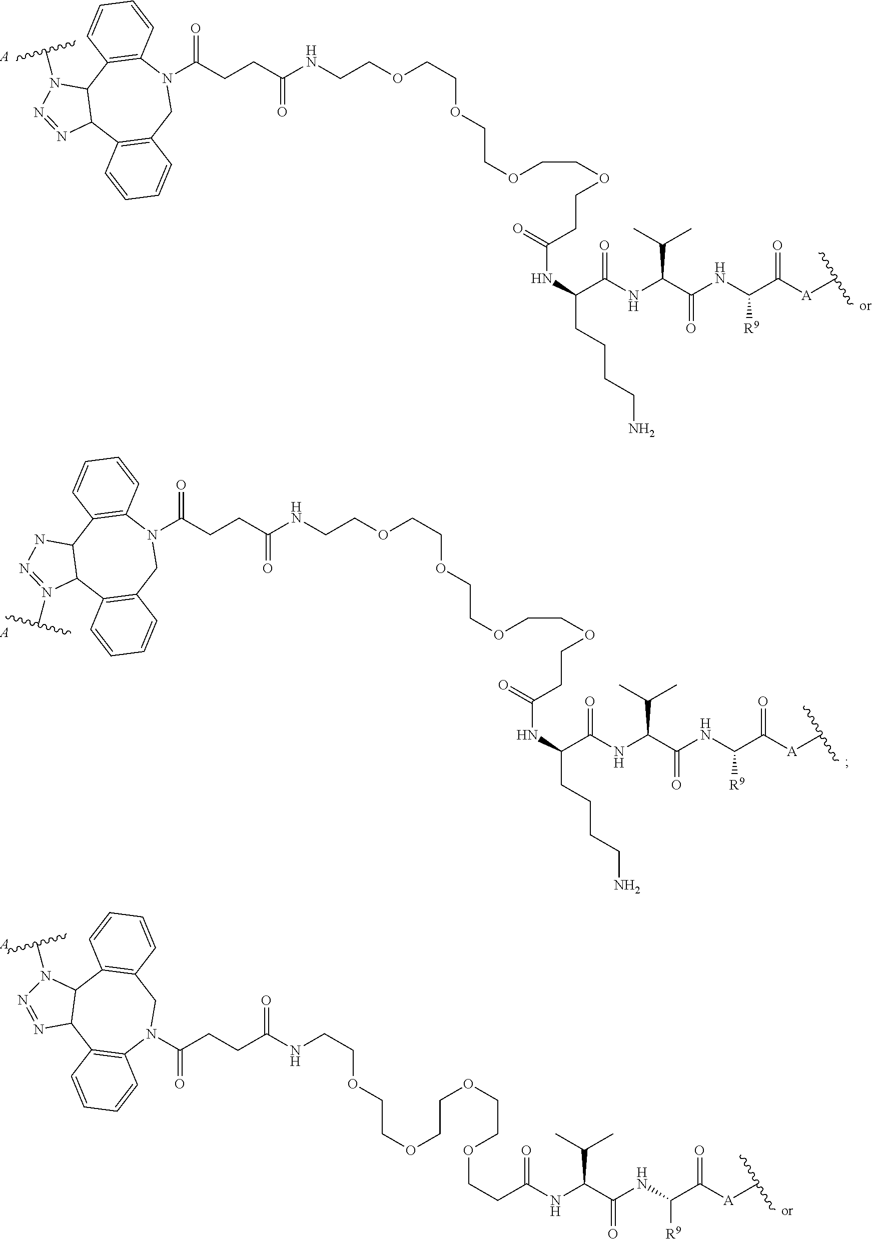

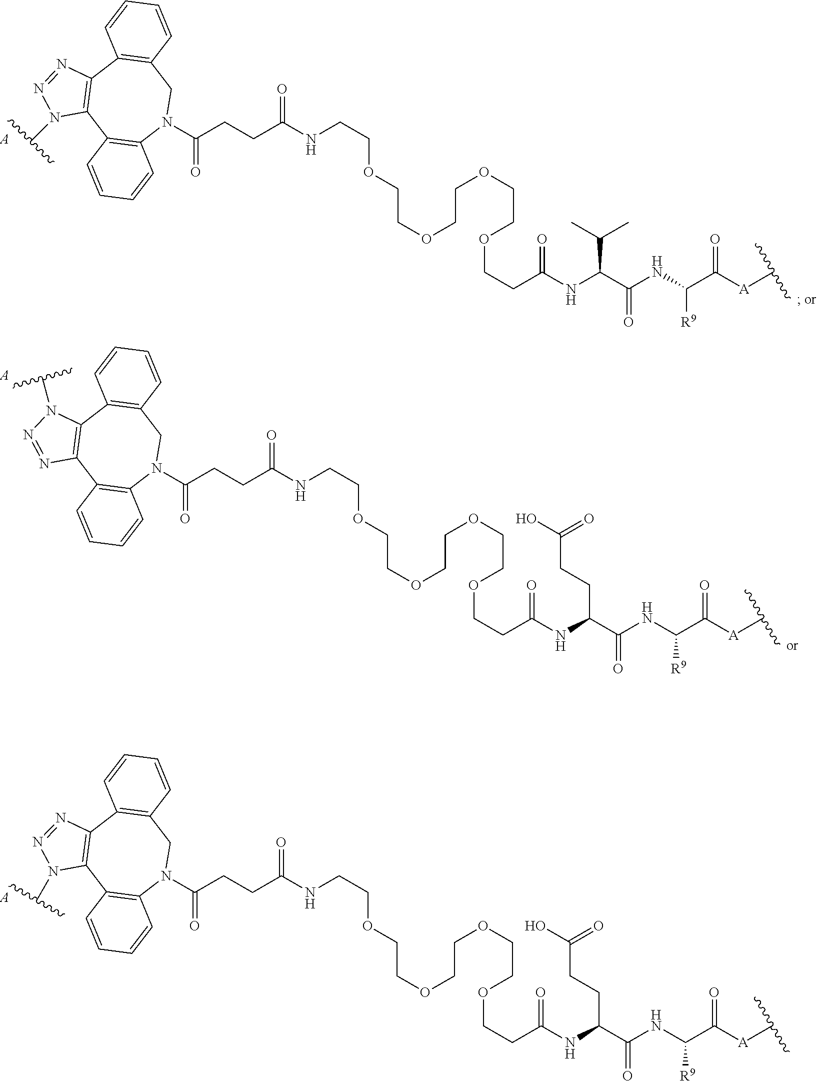

wherein:

each

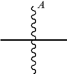

is a bond to the binding agent;

each

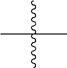

is a bond to the payload;

each R⁹ is —CH₃ or —(CH₂)₃N(H)C(O)NH₂; and

each A is —O—, —N(H)—,

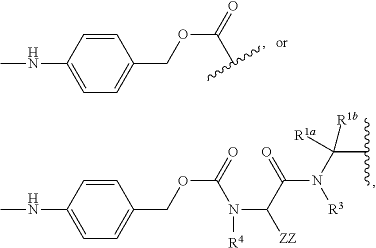

where ZZ is hydrogen, alkylene, heteroalkylene, or a side chain for an amino acid as discussed elsewhere herein. As discussed above, the bond to the binding agent can be direct, or via a spacer. In certain embodiments, the bond to the binding agent is via a PEG spacer to a glutamine residue of the binding agent.

In some embodiments, the linker is:

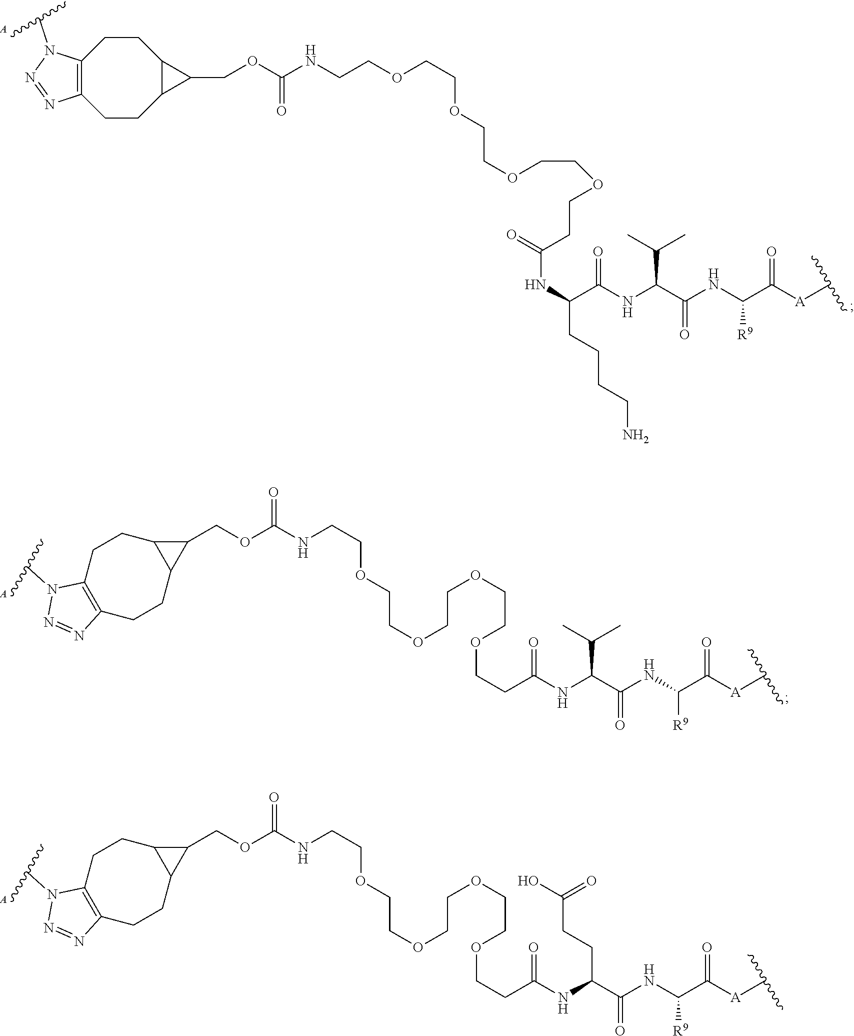

wherein:

each

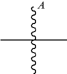

is a bond to the binding agent;

each

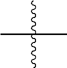

is a bond to the payload;

each R⁹ is —CH₃ or —(CH₂)₃N(H)C(O)NH₂; and

each A is —O—, —N(H)—,

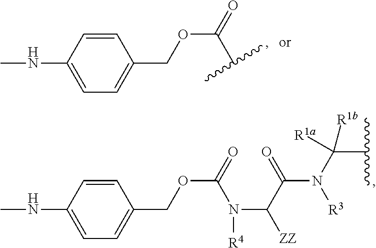

where ZZ is hydrogen, alkylene, heteroalkylene, or a side chain for an amino acid as discussed elsewhere herein. As discussed above, the bond to the binding agent can be direct, or via a spacer. In certain embodiments, the bond to the binding agent is via a PEG spacer to a glutamine residue of the binding agent.

In any of the above embodiments, the (AA)ₙ group can be modified with one or more enhancement groups. Advantageously, the enhancement group can be linked to the side chain of any amino acid in (AA)ₙ. Useful amino acids for linking enhancement groups include lysine, asparagine, aspartate, glutamine, glutamate, and citrulline. The link to the enhancement group can be a direct bond to the amino acid side chain, or the link can be indirect via a spacer and/or reactive group. Useful spacers and reactive groups include any described above. The enhancement group can be any group deemed useful by those of skill in the art. For example, the enhancement group can be any group that imparts a beneficial effect to the compound, prodrug, payload, linker-payload, or antibody conjugate including, but not limited to, biological, biochemical, synthetic, solubilizing, imaging, detecting, and reactivity effects, and the like. In certain embodiments, the enhancement group is a hydrophilic group. In certain embodiments, the enhancement group is a cyclodextrin. In certain embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose. and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the enhancement group is capable of improving solubility of the remainder of the conjugate. In certain embodiments, the alkyl, heteroalkyl, alkylenyl, or heteroalkylenyl sulfonic acid is substituted or non-substituted. In certain embodiments, the alkyl, heteroalkyl, alkylenyl, or heteroalkylenyl sulfonic acid is —(CH₂)₁₋₅SO₃H, —(CH₂)ₙ—NH—(CH₂)₁₋₅SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N((CH₂)₁₋₅C(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkylenyl sulfonic acid is —(CH₂)₁₋₅SO₃H. In another embodiment, the heteroalkyl or heteroalkylenyl sulfonic acid is —(CH₂)ₙ—NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is 1, 2, 3, 4, or 5. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5. In some embodiments, the linker is:

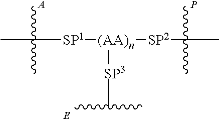

wherein:

SP¹ is a spacer;

SP² is a spacer;

SP³ is a spacer, linked to one AA of (AA)ₙ;

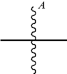

is one or more bonds to the binding agent;

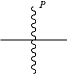

is one or more bonds to the payload;

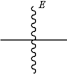

is one or more bonds to the enhancement group EG;

each AA is an amino acid; and

n is an integer from 1 to 10.

As discussed above, the bond to the binding agent can be direct, or via a spacer. In certain embodiments, the bond to the binding agent is via a PEG spacer to a glutamine residue of the binding agent.

The SP¹ spacer group is as described above. The SP² spacer group is as described above. Each (AA)ₙ group is as described above.

The SP³ spacer is a moiety that connects the (AA)ₙ moiety to the enhancement group (EG). Suitable SP³ spacers include, but are not limited to, those comprising alkylene or polyether, or both. The ends of the SP³ spacers, i.e., the portion of the SP³ spacer directly bonded to the enhancement group or an AA, can be moieties derived from reactive moieties that are used for purposes of coupling the enhancement group or an AA to the SP³ spacer during the chemical synthesis of the conjugate. In some examples, the ends of the SP³ spacers, i.e., the portion of the spacer directly bonded to the enhancement group or an AA, can be residues of reactive moieties that are used for purposes of coupling the enhancement group or an AA to the spacer during the chemical synthesis of the conjugate. In certain embodiments, SP³ is a spacer, linked to one and only one AA of (AA)ₙ. In certain embodiments, the SP³ spacer is linked to the side chain of a lysine residue of (AA)ₙ.

In some embodiments, the SP³ spacer is:

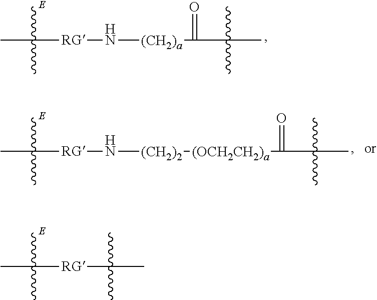

wherein:

RG′ is a reactive group residue following reaction of a reactive group RG with an enhancement agent EG;

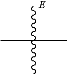

is a bond to the enhancement agent;

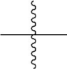

is a bond to (AA)ₙ;

a is an integer from 2 to 8; and

n is an integer from 1 to 4.

The reactive group RG can be any reactive group known to those of skill in the art to be capable of forming one or more bonds to the enhancement agent. The reactive group RG is a moiety comprising a portion in its structure that is capable of reacting with the enhancement group to form a compound of Formulae II or III. Following conjugation to the enhancement group, the reactive group becomes the reactive group residue (RG′). The reactive group RG can be any reactive group described above. Illustrative reactive groups include, but are not limited to, those that comprise haloacetyl, isothiocyanate, succinimide, N-hydroxysuccinimide, or maleimide portions that are capable of reacting with the binding agent.

In certain embodiments, reactive groups include, but are not limited to, alkynes. In certain embodiments, the alkynes are alkynes capable of undergoing 1,3-cycloaddition reactions with azides in the absence of copper catalysts such as strained alkynes. Strained alkynes are suitable for strain-promoted alkyne-azide cycloadditions (SPAAC), cycloalkynes, e.g., cyclooctynes, ane benzannulated alkynes. Suitable alkynes include, but are not limited to, dibenzoazacyclooctyne or

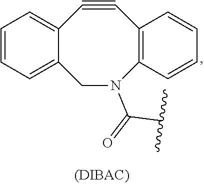 [SMILES: *C(=O)N1Cc2ccccc2C#Cc2ccccc21]

dibenzocyclooctyne or

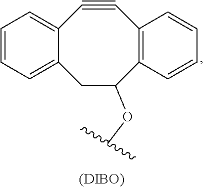 [SMILES: *OC1Cc2ccccc2C#Cc2ccccc21 ; "(DIBO)"]

biarylazacyclooctynone or

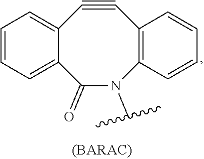 [SMILES: *N1C(=O)c2ccccc2C#Cc2ccccc21]

difluorinated cyclooctyne or

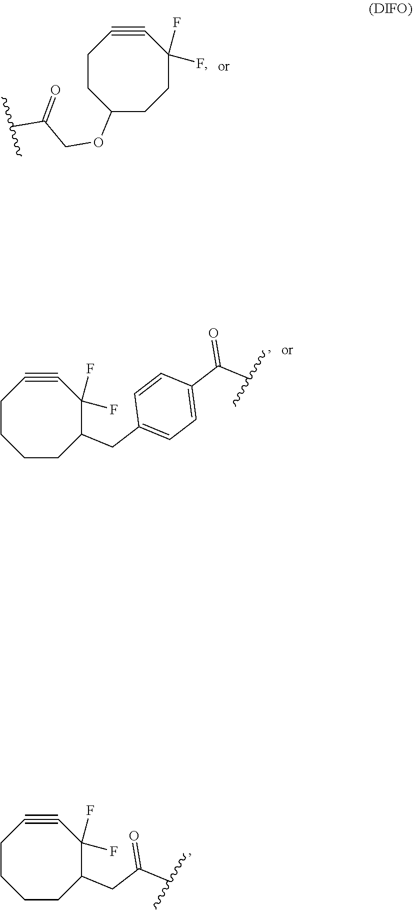 [SMILES: *C(=O)COC1CCC#CC(F)(F)CC1 ; *C(=O)c1ccc(CC2CCCCC#CC2(F)F)cc1 ; *C(=O)CC1CCCCC#CC1(F)F]

substituted, e.g., fluorinated alkynes, aza-cycloalkynes, bicycle[6.1.0]nonyne or

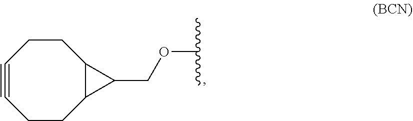 [SMILES: *OCC1C2CCC#CCCC21]

and derivatives thereof. Particularly useful alkynes include

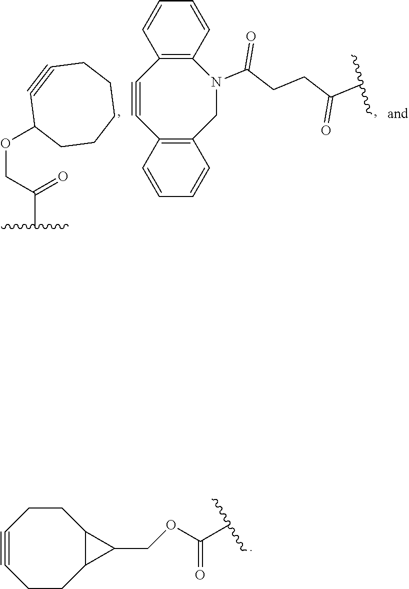

In some embodiments, the linker is:

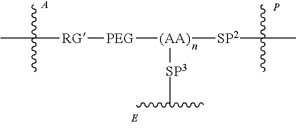

wherein:

RG′ is a reactive group residue following reaction of a reactive group RG with a binding agent;

PEG is —NH-PEG4-C(O)—;

SP² is a spacer;

SP³ is a spacer, linked to one AA residue of (AA)ₙ;

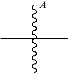

is one or more bonds to the binding agent;

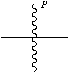

is one or more bonds to the payload;

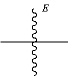

is one or more bonds to the enhancement group EG;

each AA is an amino acid residue; and

n is an integer from 1 to 10.

As discussed above, the bond to the binding agent can be direct, or via a spacer. In certain embodiments, the bond to the binding agent is via a PEG spacer to a glutamine residue of the binding agent.

In certain embodiments, the linker is:

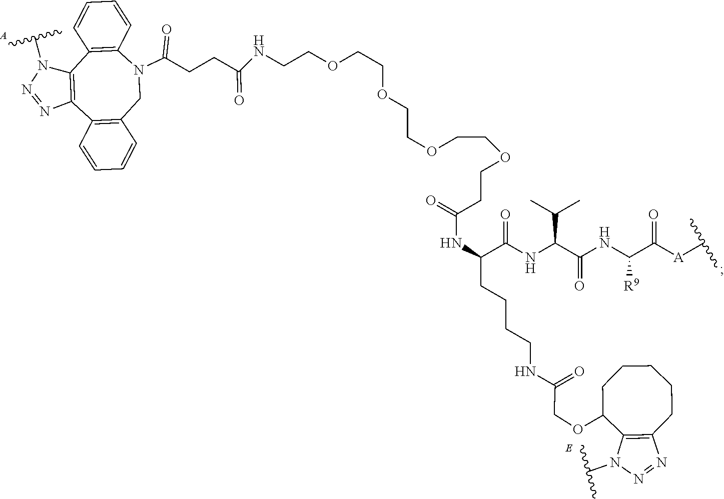

or a pharmaceutically acceptable salt, solvate, or stereoisomeric form thereof, or a regioisomer thereof, or a mixture of regioisomers thereof, wherein:

each

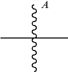

is a bond to the binding agent;

each

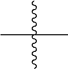

is a bond to the payload;

each

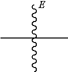

is a bond to the enhancement agent;

each R⁹ is —CH₃ or —(CH₂)₃N(H)C(O)NH₂; and

each A is —O—, —N(H)—,

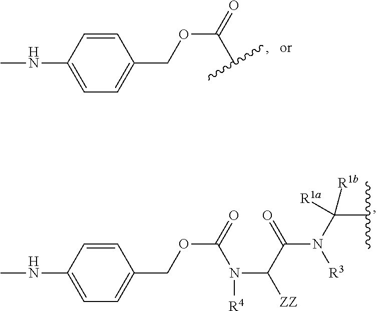

where ZZ is hydrogen, alkylene, heteroalkylene, or a side chain for an amino acid as discussed elsewhere herein. In certain embodiments, 1,3-cycloaddition or SPAAC regioisomers, or mixture of regioisomers, are derived from PEG-N₃ derivitized antibodies treated with suitable alkynes. For example, in one embodiment, the linker is:

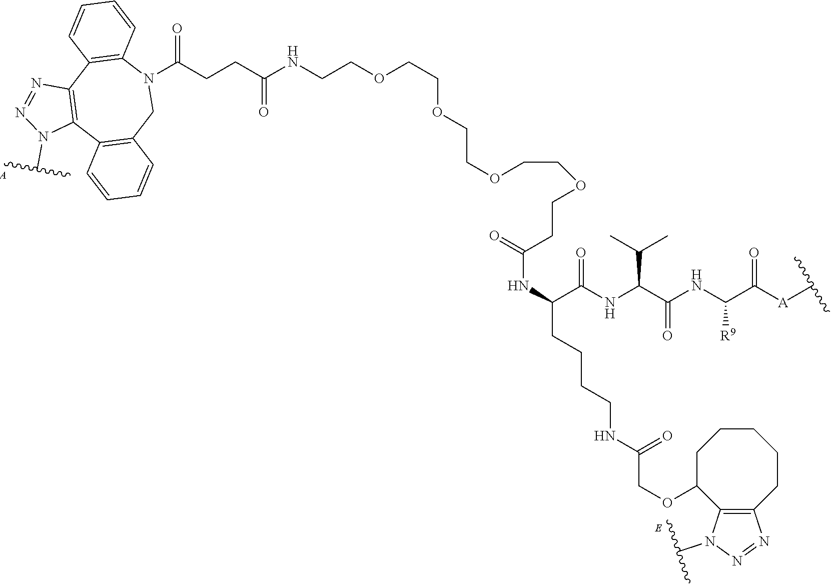

or a pharmaceutically acceptable salt, solvate, or stereoisomeric form thereof, or a regioisomer thereof, or a mixture of regioisomers thereof. By way of further example, in one embodiment, the linker is:

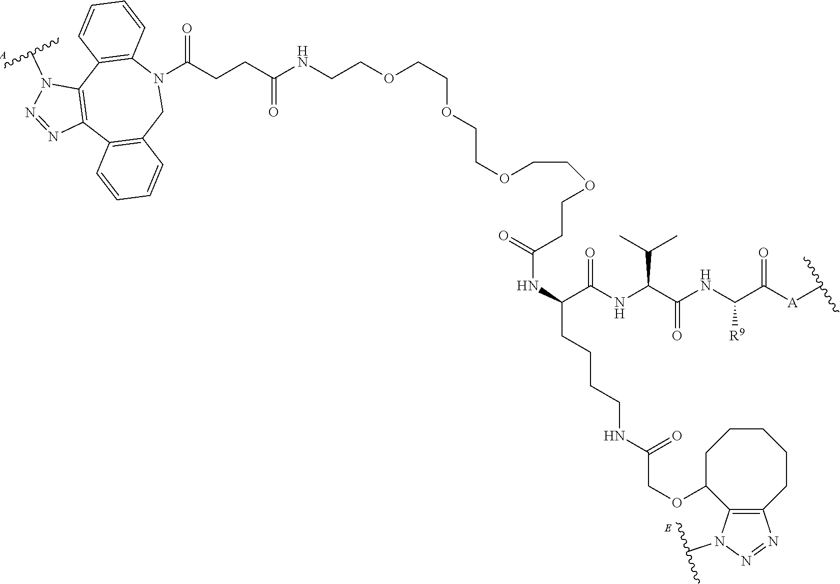

or a pharmaceutically acceptable salt, solvate, or stereoisomeric form thereof, or a regioisomer thereof, or a mixture of regioisomers thereof. By way of further example, the linker is:

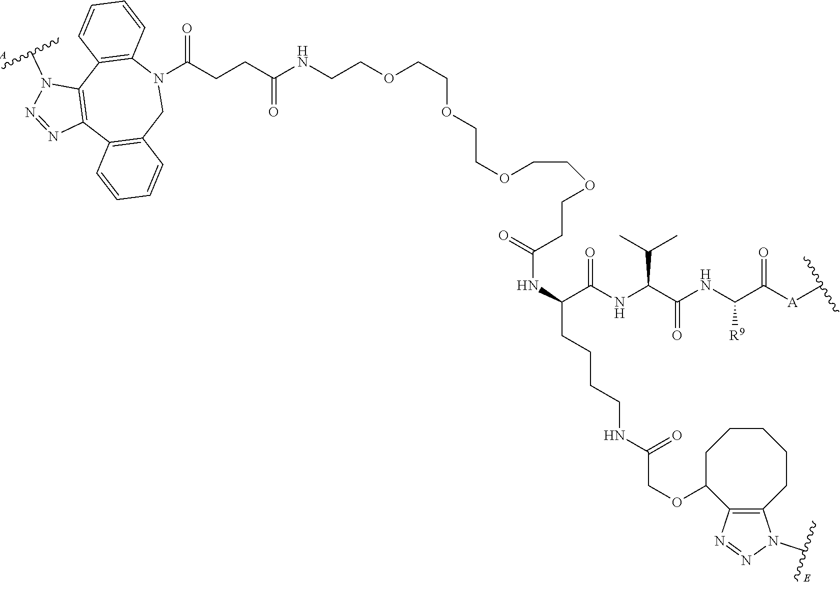

or a pharmaceutically acceptable salt, solvate, or stereoisomeric form thereof, or a regioisomer thereof, or a mixture of regioisomers thereof. By way of further example, in one embodiment, the linker is:

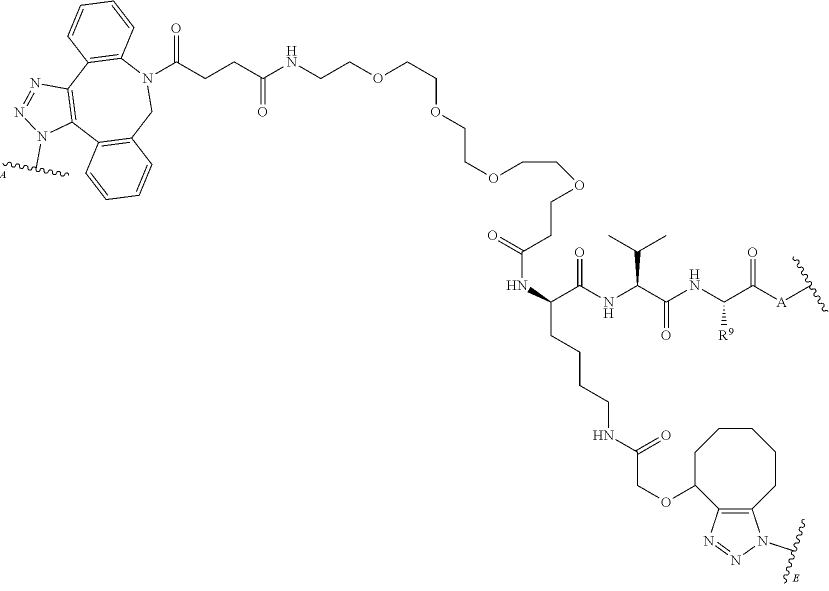

or a pharmaceutically acceptable salt, solvate, or stereoisomeric form thereof, or a regioisomer thereof, or a mixture of regioisomers thereof. As discussed above, the bond to the binding agent can be direct, or via a spacer. In certain embodiments, the bond to the binding agent is via a PEG spacer to a glutamine residue of the binding agent. In certain embodiments, the enhancement agent is a hydrophilic group. In certain embodiments, the enhancement agent is cyclodextrin. In certain embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose. and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the alkyl, heteroalkyl, alkylenyl, or heteroalkylenyl sulfonic acid is —(CH₂)₁₋₅SO₃H, —(CH₂)ₙ—NH—(CH₂)₁₋₅SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N((CH₂)₁₋₅C(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkylenyl sulfonic acid is —(CH₂)₁₋₅SO₃H. In another embodiment, the heteroalkyl or heteroalkylenyl sulfonic acid is —(CH₂)ₙ NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is 1, 2, 3, 4, or 5. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5.

In some embodiments, the linker is:

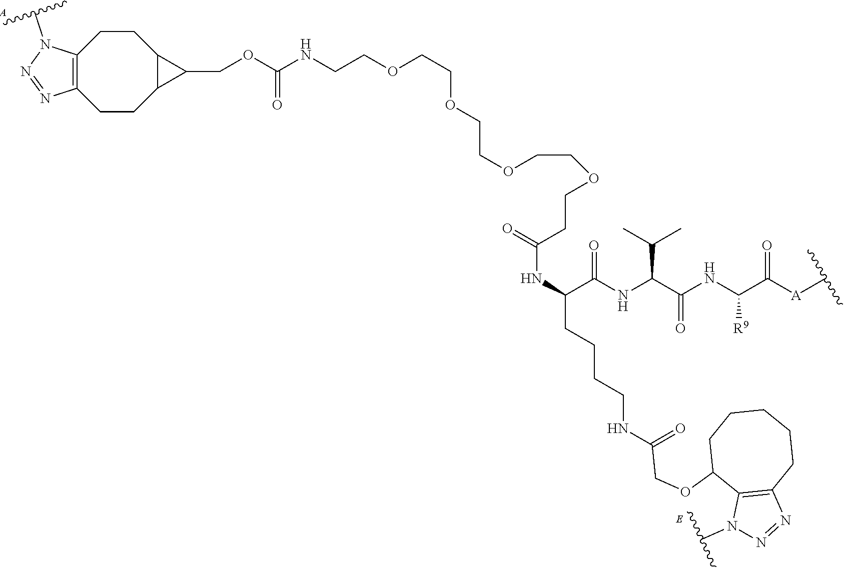

or a pharmaceutically acceptable salt, solvate, or stereoisomeric form thereof, or a regioisomer thereof, or mixture of regioisomers thereof, wherein:

each

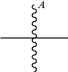

is a bond to the binding agent;

each

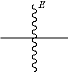

is a bond to the enhancement agent;

each

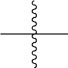

is a bond to the payload;
each R⁹ is —CH₃ or —(CH₂)₃N(H)C(O)NH₂; and

each A is —O—, —N(H)—,

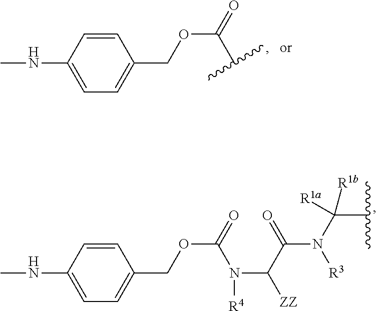

where ZZ is hydrogen, alkylene, heteroalkylene, or a side chain for an amino acid as discussed elsewhere herein. As discussed above, the bond to the binding agent can be direct, or via a spacer. In certain embodiments, the bond to the binding agent is via a PEG spacer to a glutamine residue of the binding agent. In certain embodiments, the enhancement agent is a hydrophilic group. In certain embodiments, the enhancement agent is cyclodextrin. In certain embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose. and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the alkyl, heteroalkyl, alkylenyl, or heteroalkylenyl sulfonic acid is —(CH₂)₁₋₅SO₃H, —(CH₂)ₙ—NH—(CH₂)₁₋₅SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N((CH₂)₁₋₅C(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkylenyl sulfonic acid is —(CH₂)₁₋₅SO₃H. In another embodiment, the heteroalkyl or heteroalkylenyl sulfonic acid is —(CH₂)ₙ—NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is 1, 2, 3, 4, or 5. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5.

In some embodiments, the linker is:

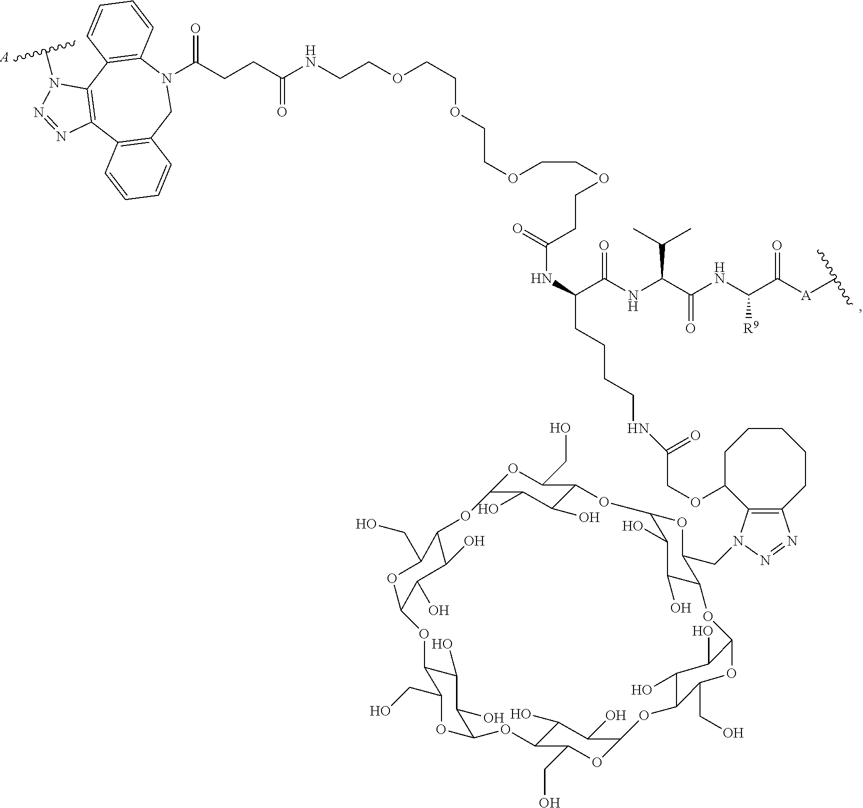

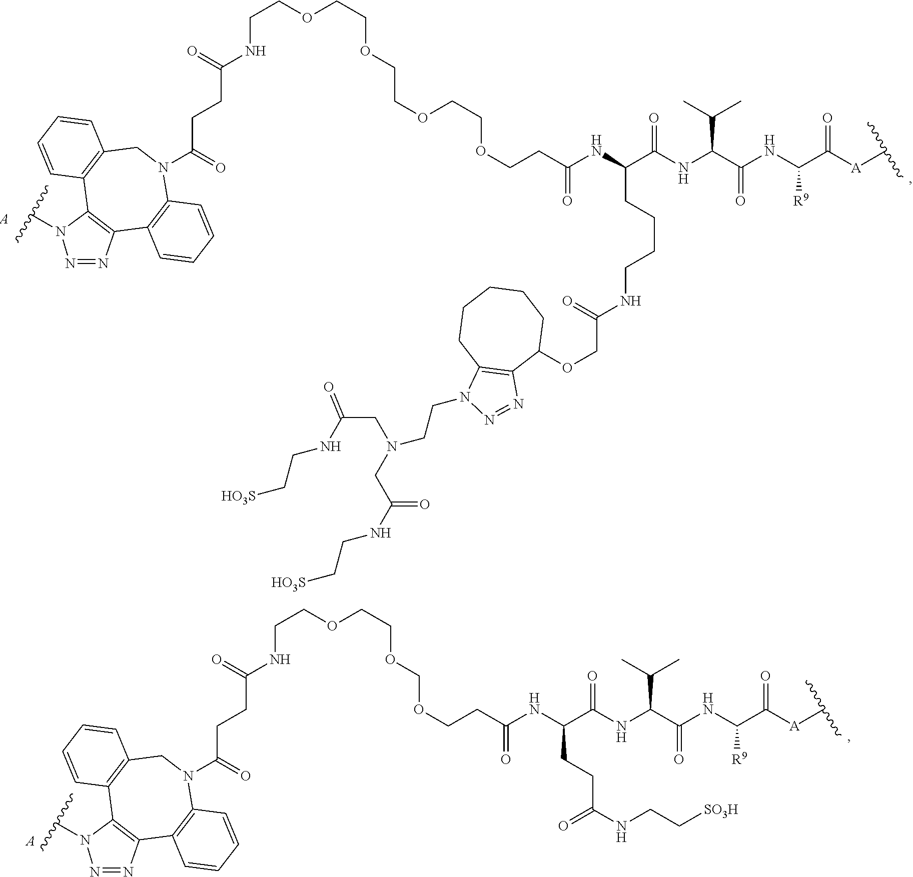

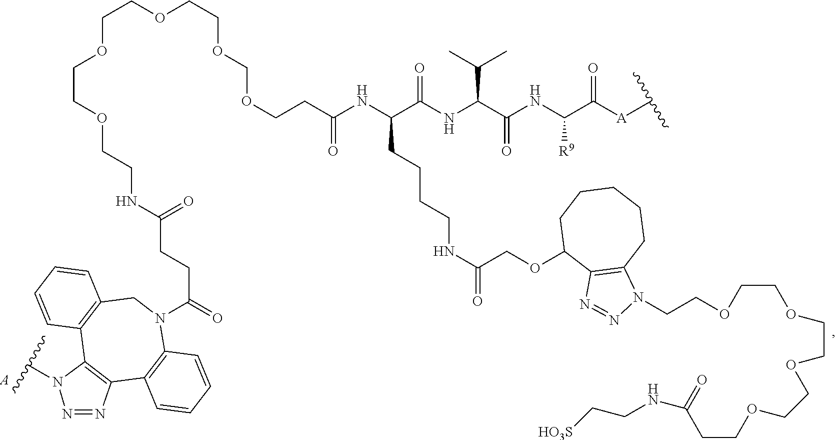

or a pharmaceutically acceptable salt, solvate, or stereoisomeric form thereof, or a regioisomer thereof, or mixture of regioisomers thereof, wherein:

each

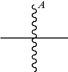

is a bond to the binding agent;

each

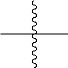

is a bond to the payload;

R⁹ is —CH₃ or —(CH₂)₃N(H)C(O)NH₂; and

A is —O—, —N(H)—,

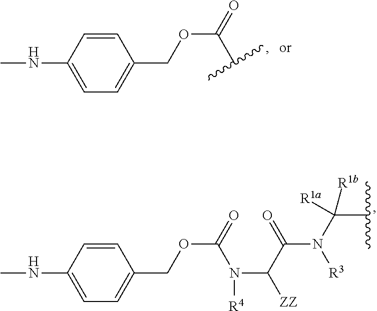

where ZZ is hydrogen, alkylene, heteroalkylene, or a side chain for an amino acid as discussed elsewhere herein. As discussed above, the bond to the binding agent can be direct, or via a spacer. In certain embodiments, the bond to the binding agent is via a PEG spacer to a glutamine residue of the binding agent.

In some embodiments, the linker is:

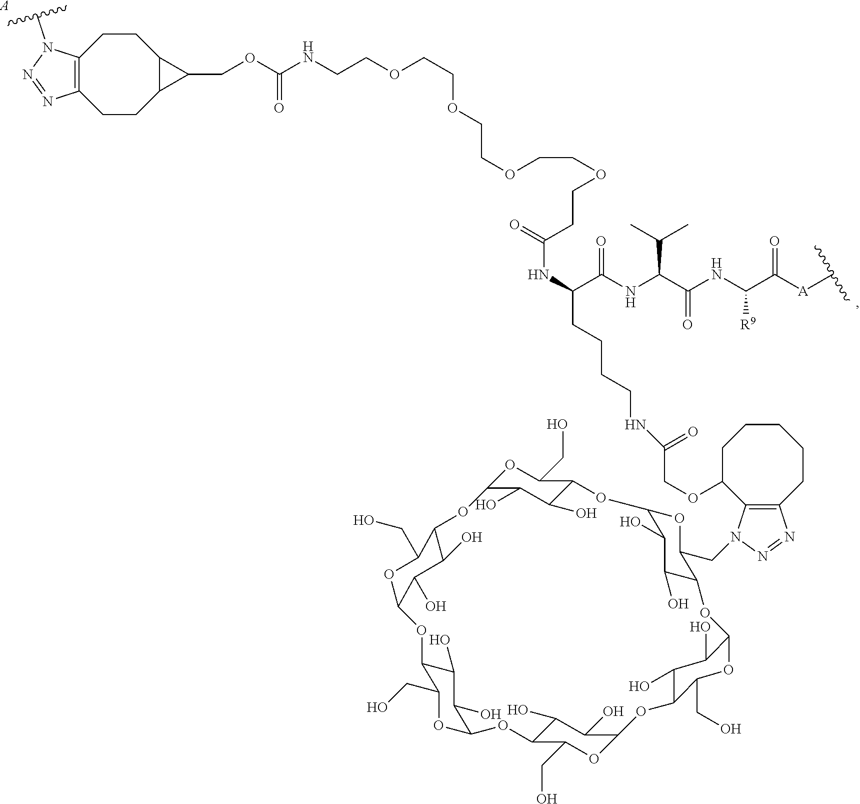

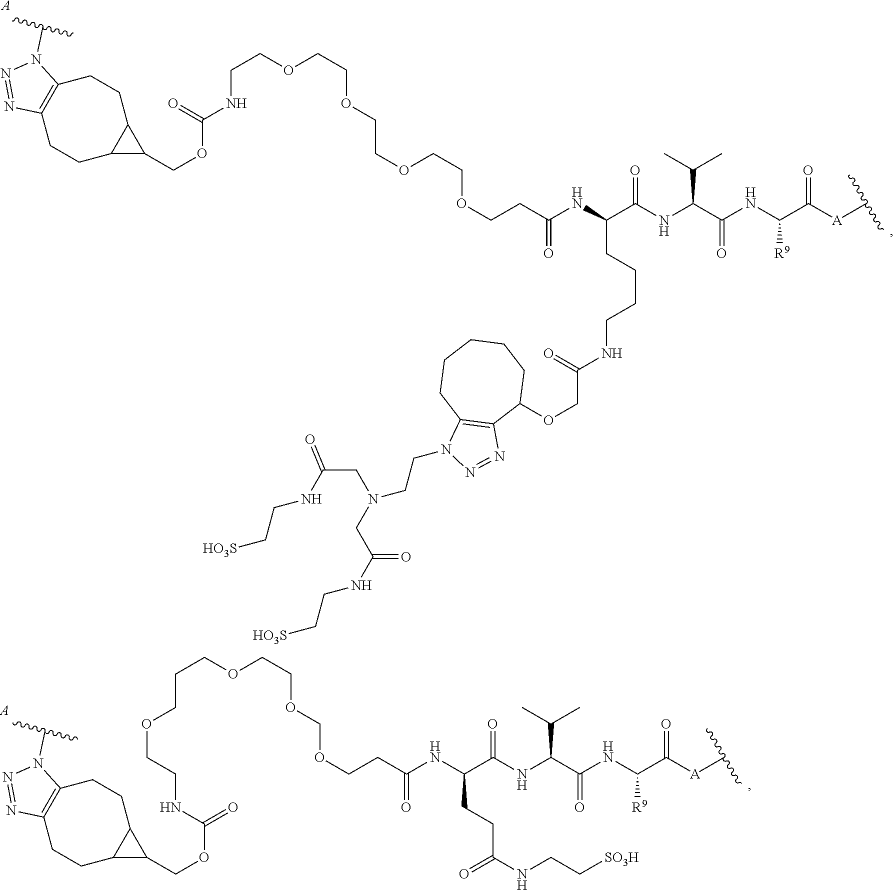

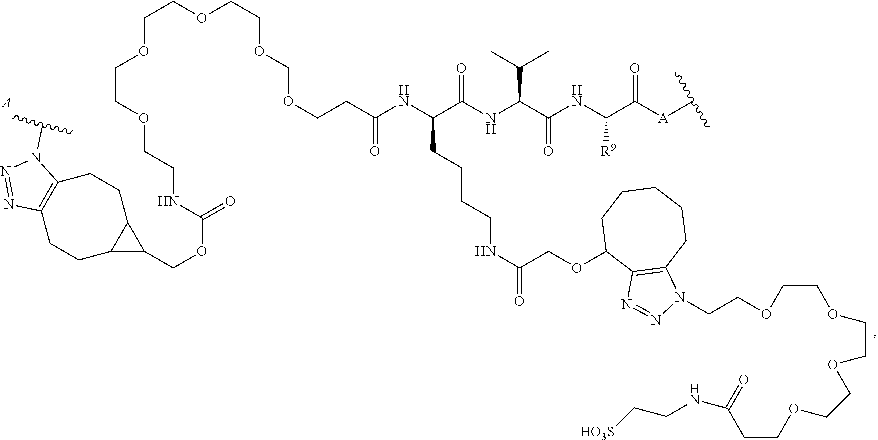

or a pharmaceutically acceptable salt, solvate, or stereoisomeric form thereof, or a regioisomer thereof, or mixture of regioisomers thereof, wherein:

each

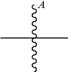

is a bond to the binding agent;

each

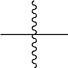

is a bond to the payload;

R⁹ is —CH₃ or —(CH₂)₃N(H)C(O)NH₂; and

A is —O—, —N(H)—,

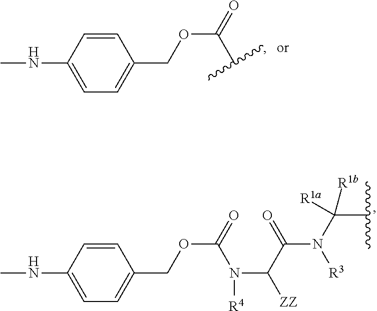

where ZZ is hydrogen, alkylene, heteroalkylene, or a side chain for an amino acid as discussed elsewhere herein. As discussed above, the bond to the binding agent can be direct, or via a spacer. In certain embodiments, the bond to the binding agent is via a PEG spacer to a glutamine residue of the binding agent.

In some embodiments, the linker is:

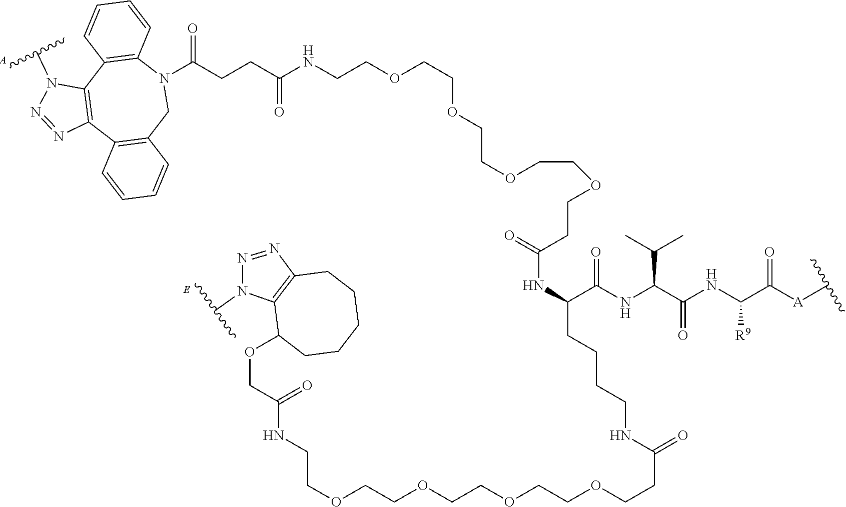

or a pharmaceutically acceptable salt, solvate, or stereoisomeric form thereof, or a regioisomer thereof, or mixture of regioisomers thereof, wherein:

each

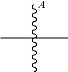

is a bond to the binding agent;

each

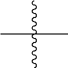

is a bond to the payload;

each

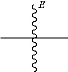

is a bond to the enhancement group;

each R⁹ is —CH₃ or —(CH₂)₃N(H)C(O)NH₂; and

each A is —O—, —N(H)—,

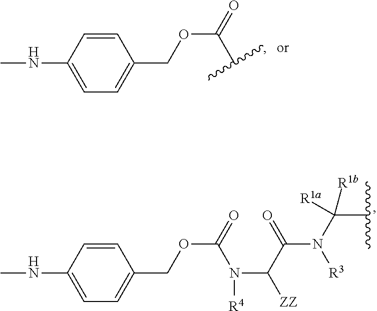

where ZZ is hydrogen, alkylene, heteroalkylene, or a side chain for an amino acid as discussed elsewhere herein. As discussed above, the bond to the binding agent can be direct, or via a spacer. In certain embodiments, the bond to the binding agent is via a PEG spacer to a glutamine residue of the binding agent. In certain embodiments, the enhancement agent is a hydrophilic group. In certain embodiments, the enhancement agent is cyclodextrin. In certain embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose. and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the alkyl, heteroalkyl, alkylenyl, or heteroalkylenyl sulfonic acid —(CH₂)₁₋₅SO₃H, —(CH₂)ₙ—NH—(CH₂)₁₋₅SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N((CH₂)₁₋₅C(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkylenyl sulfonic acid is —(CH₂)₁₋₅SO₃H. In another embodiment, the heteroalkyl or heteroalkylenyl sulfonic acid is —(CH₂)ₙ—NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is 1, 2, 3, 4, or 5. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5.

In some embodiments, the linker is:

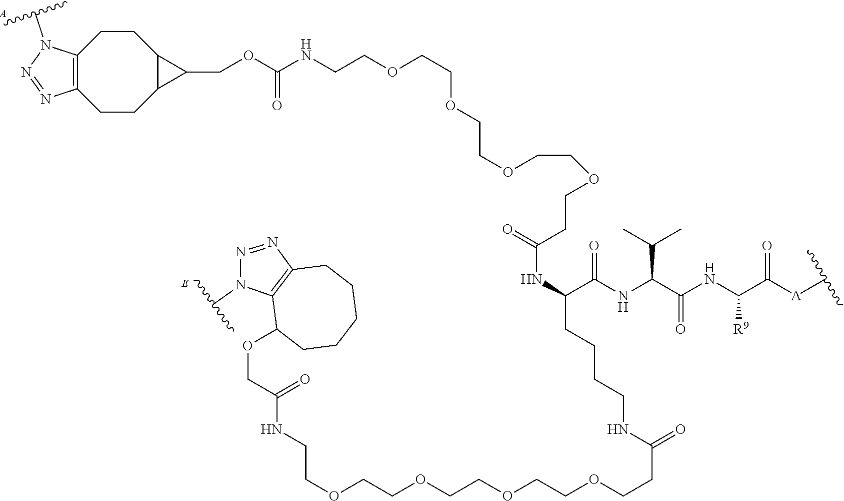

or a pharmaceutically acceptable salt, solvate, or stereoisomeric form thereof, or a regioisomer thereof, or mixture of regioisomers thereof, wherein:

each

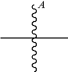

is a bond to the binding agent;

each

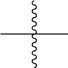

is a bond to the payload;

each R⁹ is —CH₃ or —(CH₂)₃N(H)C(O)NH₂; and

each A is —O—, —N(H)—,

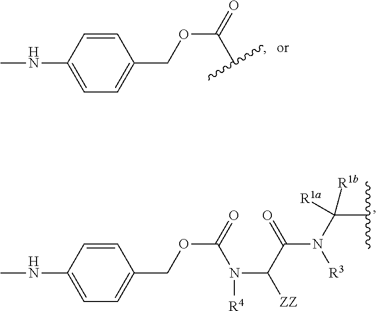

where ZZ is hydrogen, alkylene, heteroalkylene, or a side chain for an amino acid as discussed elsewhere herein. As discussed above, the bond to the binding agent can be direct, or via a spacer. In certain embodiments, the bond to the binding agent is via a PEG spacer to a glutamine residue of the binding agent. In certain embodiments, the enhancement agent is a hydrophilic group. In certain embodiments, the enhancement agent is cyclodextrin. In certain embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose. and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the alkyl, heteroalkyl, alkylenyl, or heteroalkylenyl sulfonic acid is —(CH₂)₁₋₅SO₃H, —(CH₂)ₙ—NH—(CH₂)₁₋₅SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N((CH₂)₁₋₅C(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkylenyl sulfonic acid is —(CH₂)₁₋₅SO₃H. In another embodiment, the heteroalkyl or heteroalkylenyl sulfonic acid is —(CH₂)ₙ—NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is 1, 2, 3, 4, or 5. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5.

In some embodiments, the linker is:

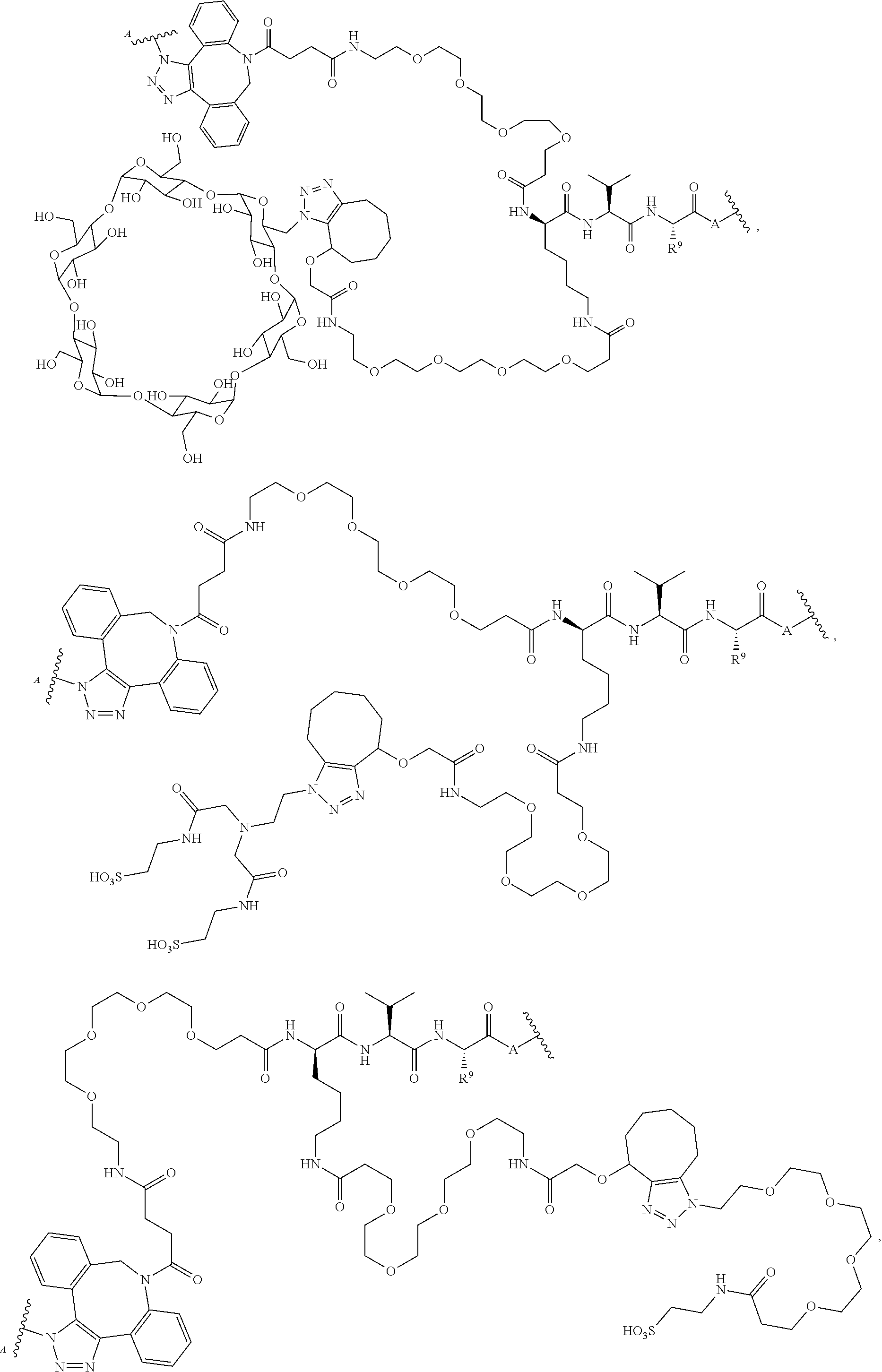

or a pharmaceutically acceptable salt, solvate, or stereoisomeric form thereof, or a regioisomer thereof, or mixture of regioisomers thereof, wherein:

each

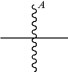

is a bond to the binding agent;

each

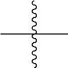

is a bond to the payload;

R⁹ is —CH₃ or —(CH₂)₃N(H)C(O)NH₂; and

A is —O—, —N(H)—,

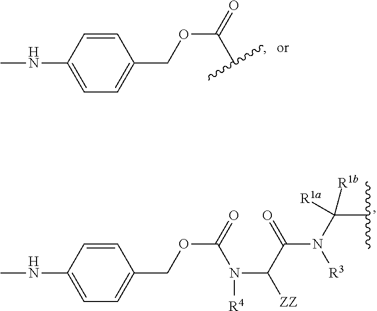

where ZZ is hydrogen, alkylene, heteroalkylene, or a side chain for an amino acid as discussed elsewhere herein. As discussed above, the bond to the binding agent can be direct, or via a spacer. In certain embodiments, the bond to the binding agent is via a PEG spacer to a glutamine residue of the binding agent.

In some embodiments, the linker is:

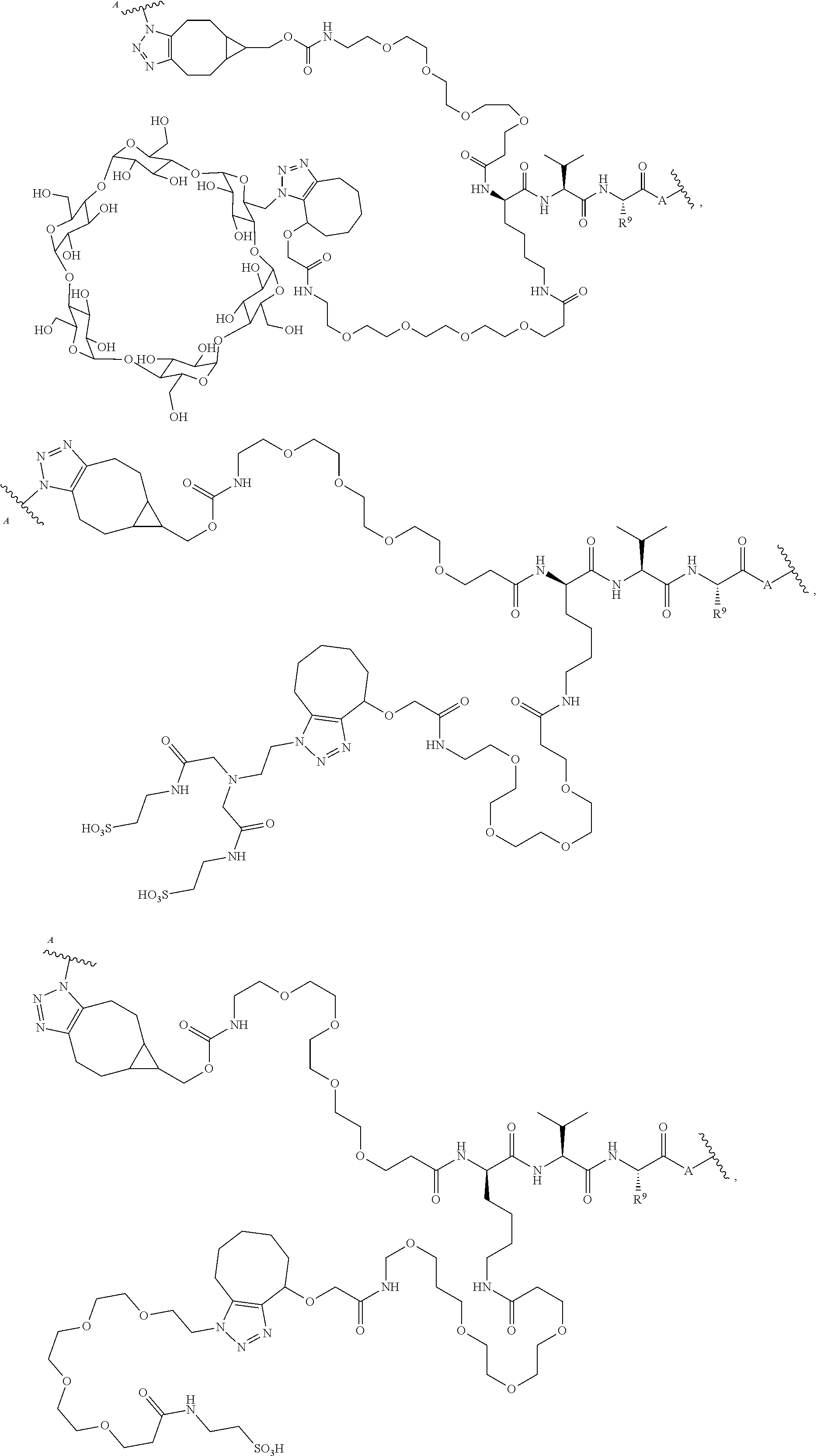

or a pharmaceutically acceptable salt, solvate, or stereoisomeric form thereof, or a regioisomer thereof, or mixture of regioisomers thereof, wherein:

each

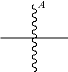

is a bond to the binding agent;

each

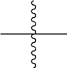

is a bond to the payload;

R⁹ is —CH₃ or —(CH₂)₃N(H)C(O)NH₂; and

A is —O—, —N(H)—,

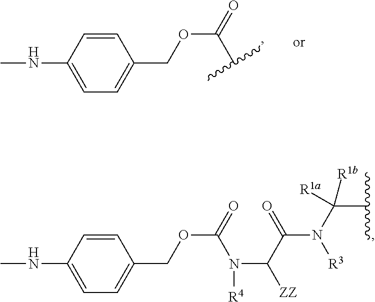

where ZZ is hydrogen, alkylene, heteroalkylene, or a side chain for an amino acid as discussed elsewhere herein. As discussed above, the bond to the binding agent can be direct, or via a spacer. In certain embodiments, the bond to the binding agent is via a PEG spacer to a glutamine residue of the binding agent.

### Linker-Payloads

In certain embodiments, linker-payloads include any specific compound, prodrug, or payload embraced by any one or more of Formulae I, Ia, Iaa, Iaaa, Ib, Ibb, and Ibbb above, bonded to a linker, wherein the linker(s) described herein include a moiety that is reactive with an antibody or antigen binding fragment thereof described herein. In particular embodiments, the linker is bonded to a primary or secondary nitrogen in any one or more of Formulae I, Ia, Iaa, Iaaa, Ib, Ibb, Ibbb, or IV. In one embodiment of Formula II, when D* is a residue of a biologically active compound comprising hydroxyl, amino, or thiol, then the biologically active compound or residue thereof is an anti-inflammatory biologically active compound or residue thereof. In another embodiment of Formula II, the anti-inflammatory biologically active compound is a steroid or a residue thereof. In another embodiment of Formula II, the anti-inflammatory biologically active compound is an LXR agonist or a residue thereof. In one embodiment, the linker-payload has a structure of Formula IIa:

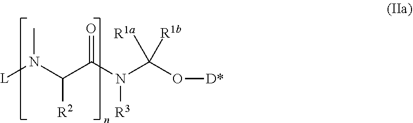

or a pharmaceutically acceptable salt thereof, wherein L is a linker comprising a moiety reactive with an antibody or an antigen binding fragment thereof; R^{1a} and R^{1b} are, independently, hydrogen, alkyl, alkoxy, alkenyl, alkynyl, aryl, arylalkyl, heteroaryl, or alkylene, wherein when R^{1a} is alkylene, the alkylene is further bonded to R³ to form a 4-, 5-, or 6-membered heterocyclyl; R² is hydrogen or an amino acid side chain; R³ is hydrogen, alkyl, or alkylene, wherein when R³ is alkylene, the alkylene is further bonded to R^{1a} to form the 4-, 5-, or 6-membered heterocyclyl; D* is acyl, or a residue of a biologically active compound comprising hydroxyl; and n is zero, one, two, three, four, five, or six. In another embodiment, the linker-payload has a structure of Formula IIaa:

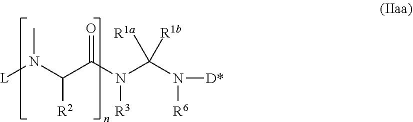

or a pharmaceutically acceptable salt thereof, wherein L is a linker comprising a moiety reactive with an antibody or an antigen binding fragment thereof; R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or hetereoalkylene is further bonded to R³ to form a 4-, 5-, or 6-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; R⁶ is hydrogen or alkyl; D* is acyl, or a residue of a biologically active compound comprising hydroxyl, amino, or thiol; and n is zero, one, two, three, four, or five. In another embodiment, the linker payload has a structure of Formula IIaaa:

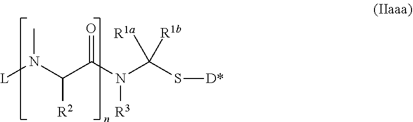

or a pharmaceutically acceptable salt thereof, wherein L is a linker comprising a moiety reactive with an antibody or an antigen binding fragment thereof; R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or hetereoalkylene is further bonded to R³ to form a 4-, 5-, or 6-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; D* is acyl, or a residue of a biologically active compound comprising hydroxyl, amino, or thiol; and n is zero, one, two, three, four, or five. In another embodiment, the linker-payload has a structure of Formula IIb:

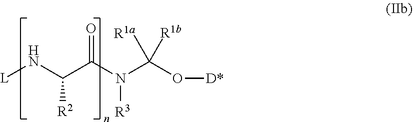

or a pharmaceutically acceptable salt thereof, wherein L is a linker comprising a moiety reactive with an antibody or an antigen binding fragment thereof; R^{1a} and R^{1b} are, independently, hydrogen, alkyl, alkoxy, alkenyl, alkynyl, aryl, arylalkyl, heteroaryl, or alkylene, wherein when R^{1a} is alkylene, the alkylene is further bonded to R³ to form a 4-, 5-, or 6-membered heterocyclyl; R² is hydrogen or an amino acid side chain; R³ is hydrogen, alkyl, or alkylene, wherein when R³ is alkylene, the alkylene is further bonded to R^{1a} to form the 4-, 5-, or 6-membered heterocyclyl; D* is acyl, or a residue of a biologically active compound comprising hydroxyl; and n is zero, one, two, three, four, five, or six. In another embodiment, the linker-payload has a structure of Formula IIbb:

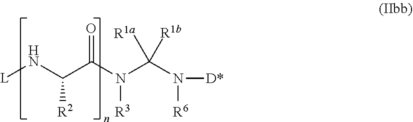

or a pharmaceutically acceptable salt thereof, wherein L is a linker comprising a moiety reactive with an antibody or an antigen binding fragment thereof; R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or hetereoalkylene is further bonded to R³ to form a 4-, 5-, or 6-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; R⁶ is hydrogen or alkyl; D* is acyl, or a residue of a biologically active compound comprising hydroxyl, amino, or thiol; and n is zero, one, two, three, four, or five. In another embodiment, the linker-payload has a structure of Formula IIbbb:

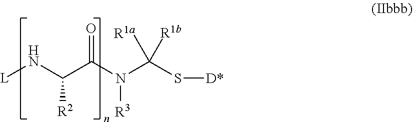

or a pharmaceutically acceptable salt thereof, wherein L is a linker comprising a moiety reactive with an antibody or an antigen binding fragment thereof; R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or hetereoalkylene is further bonded to R³ to form a 4-, 5-, or 6-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; D* is acyl, or a residue of a biologically active compound comprising hydroxyl, amino, or thiol; and n is zero, one, two, three, four, or five. In any of the foregoing IIa, IIaa, IIaaa, IIb, IIbb, and IIbbb embodiments, the linker further comprises

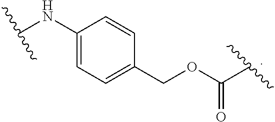 [SMILES: *Nc1ccc(COC(*)=O)cc1]

embodiment, the linker-payload has a structure of Formula IV.

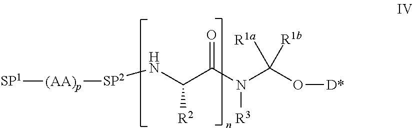

or a pharmaceutically acceptable salt thereof, wherein R^{1a}, R^{1b}, R², R³, D*, and n are as described in any of the embodiments disclosed herein, and wherein SP¹ and SP², when present, are spacer groups wherein SP¹ further comprises a moiety reactive with an antibody or an antigen binding fragment thereof; each AA is an amino acid; and p is an integer from 1 to 10. In one embodiment, the linker-payload has a structure of Formula IVa:

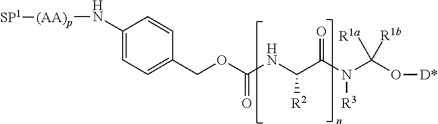

or a pharmaceutically acceptable salt thereof, wherein R^{1a}, R^{1b}, R², R³, D*, and n are as described in any of the embodiments disclosed herein, and wherein SP¹, when present, is a spacer group wherein SP¹ further comprises a moiety reactive with an antibody or an antigen binding fragment thereof; each AA is an amino acid; and p is an integer from 1 to 10. In certain embodiments of Formula IV or Formula IVa, R^{1a} and R^{1b} are hydrogen; R² is hydrogen or methyl; R³ is hydrogen; and n is two. In another embodiment of Formula IV or Formula IVa, D* is hydrogen. In another embodiment of Formula IV or Formula IVa, D* is a residue of a biologically active compound comprising hydroxyl. In another embodiment of Formula IV or Formula IVa, the linker payload is selected from the group consisting of

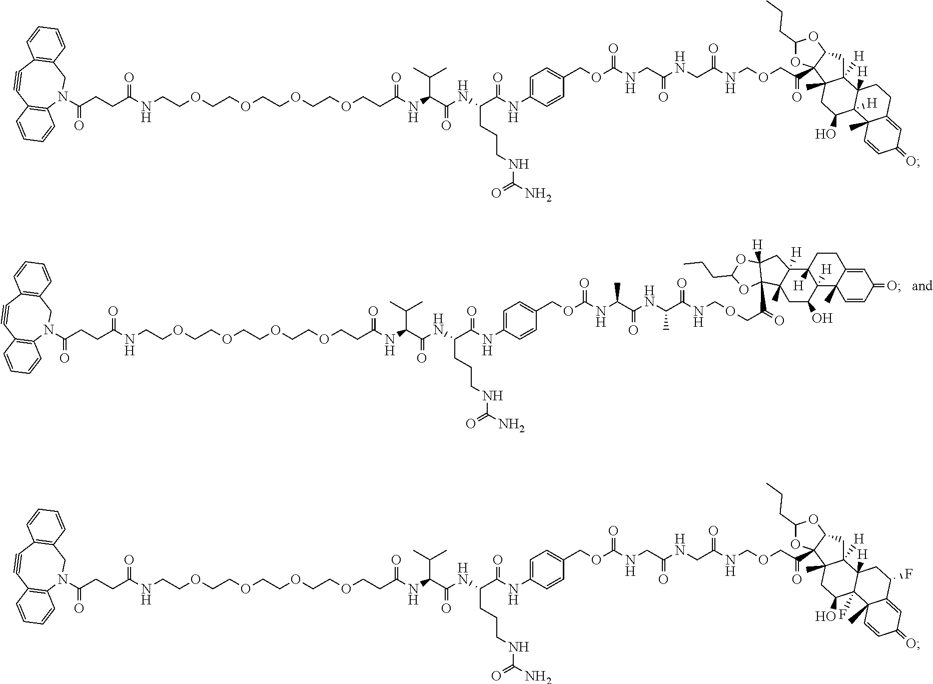

or a pharmaceutically acceptable salt thereof. In certain embodiments within this paragraph, all diastereomers are contemplated. For example, in one embodiment, the stereochemistry at the acetal is undefined or racemic. By way of further example, in one embodiment, the stereochemistry at the acetal is (R)-. By way of further example, in one embodiment, the stereochemistry at the acetal is (S)-. By way of further example, in one embodiment, the stereochemistry at the acetal is (R)- in excess of (S)-. By way of further example, in one embodiment, the stereochemistry at the acetal is (S)- in excess of (R)-. In any of the embodiments in this paragraph, p is 1, 2, 3, 4, 5, 6, 7, 8, 9, or 10. In any of the embodiments in this paragraph, n is 1, 2, 3, 4, 5, 6, 7, 8, 9, 10, 11, 12, 13, 14, or 15.

In certain embodiments of Formula IV or Formula IVa, R^{1a} and R^{1b} are hydrogen; R² is hydrogen, methyl, or —CH₂Ph; R³ is hydrogen or alkyl; and n is one. In another embodiment of Formula IV or Formula IVa, D* is hydrogen. In another embodiment of Formula IV or Formula IVa, D* is a residue of a biologically active compound comprising hydroxyl. In another embodiment of Formula IV or Formula IVa, the linker-payload is selected from the group consisting of

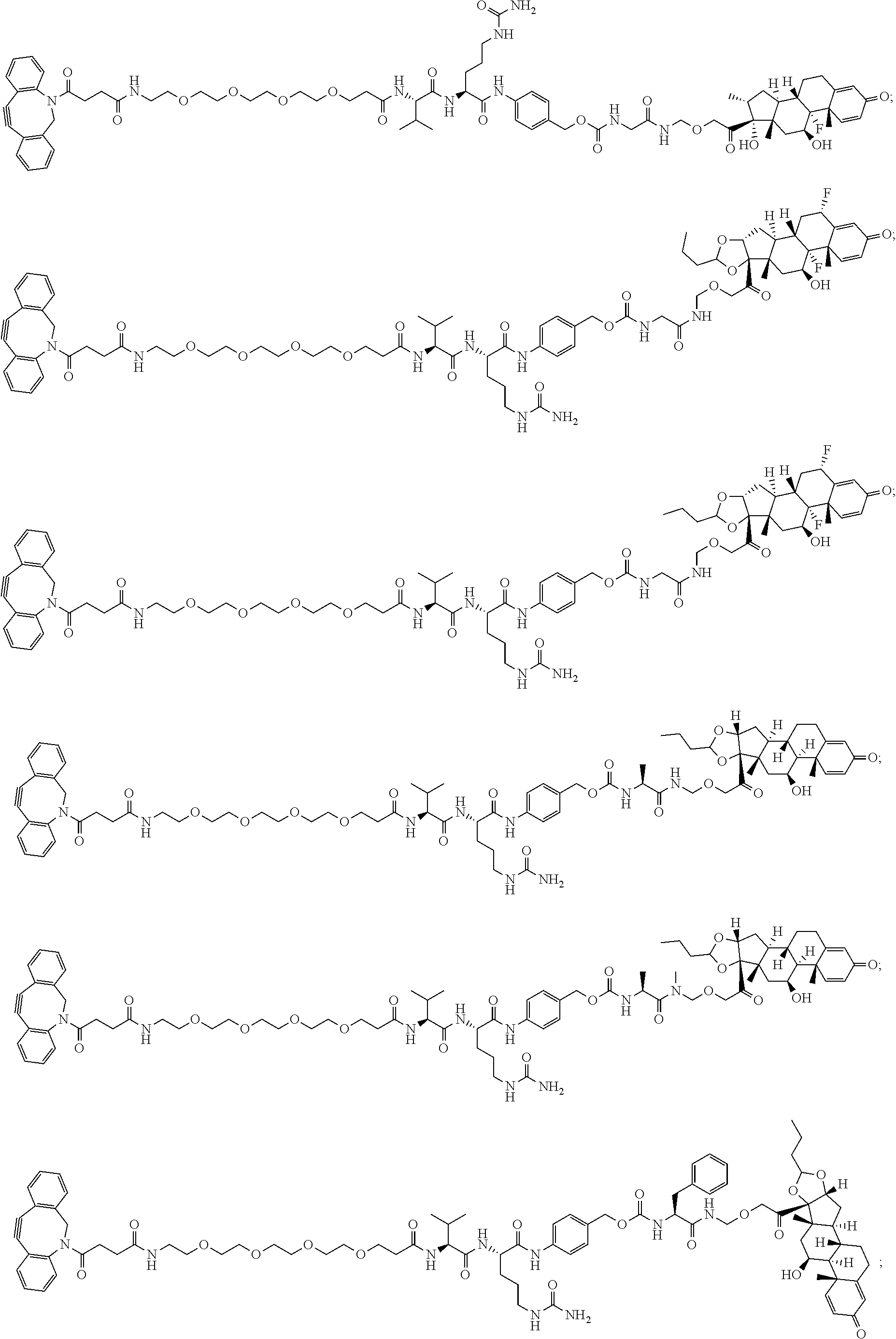

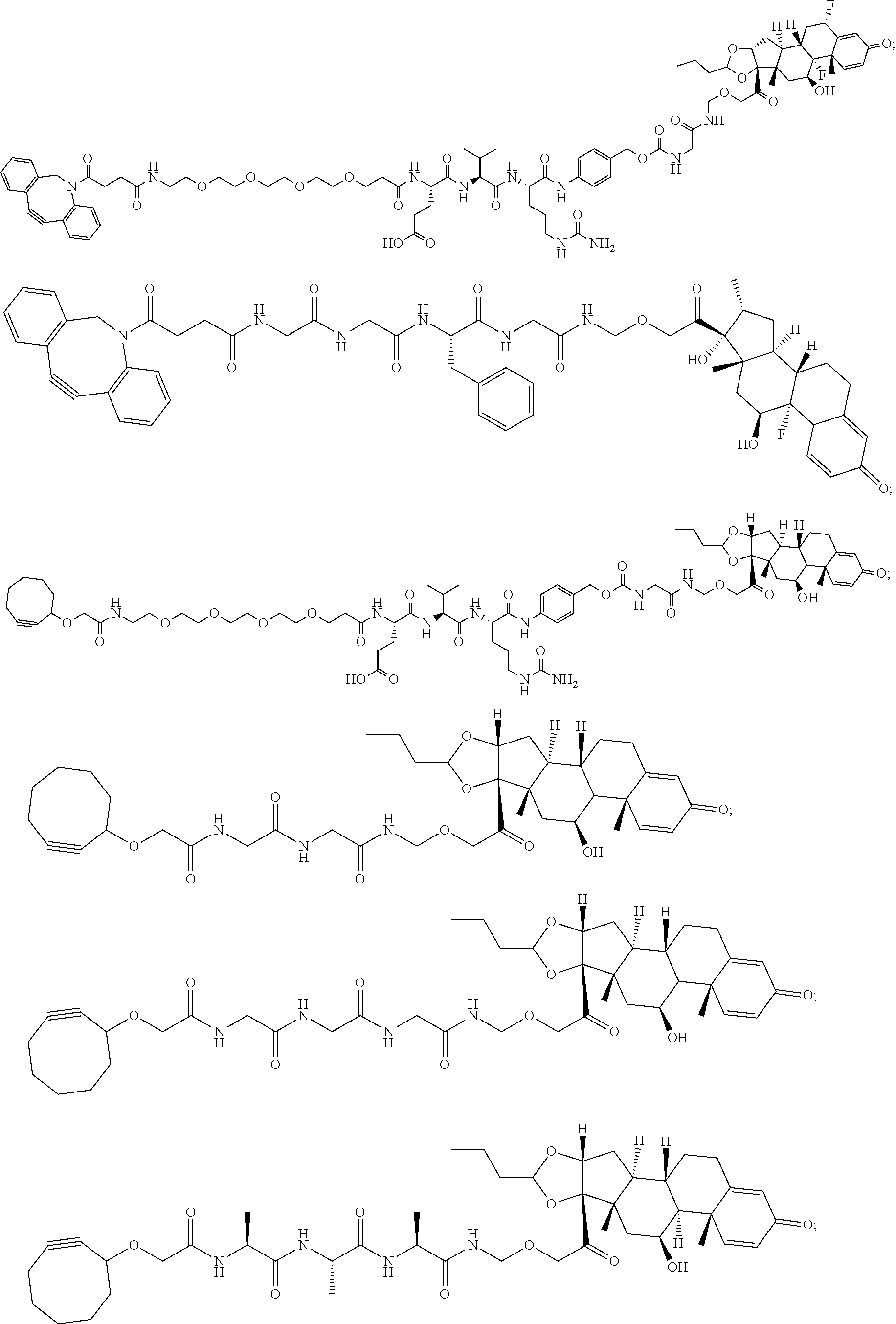

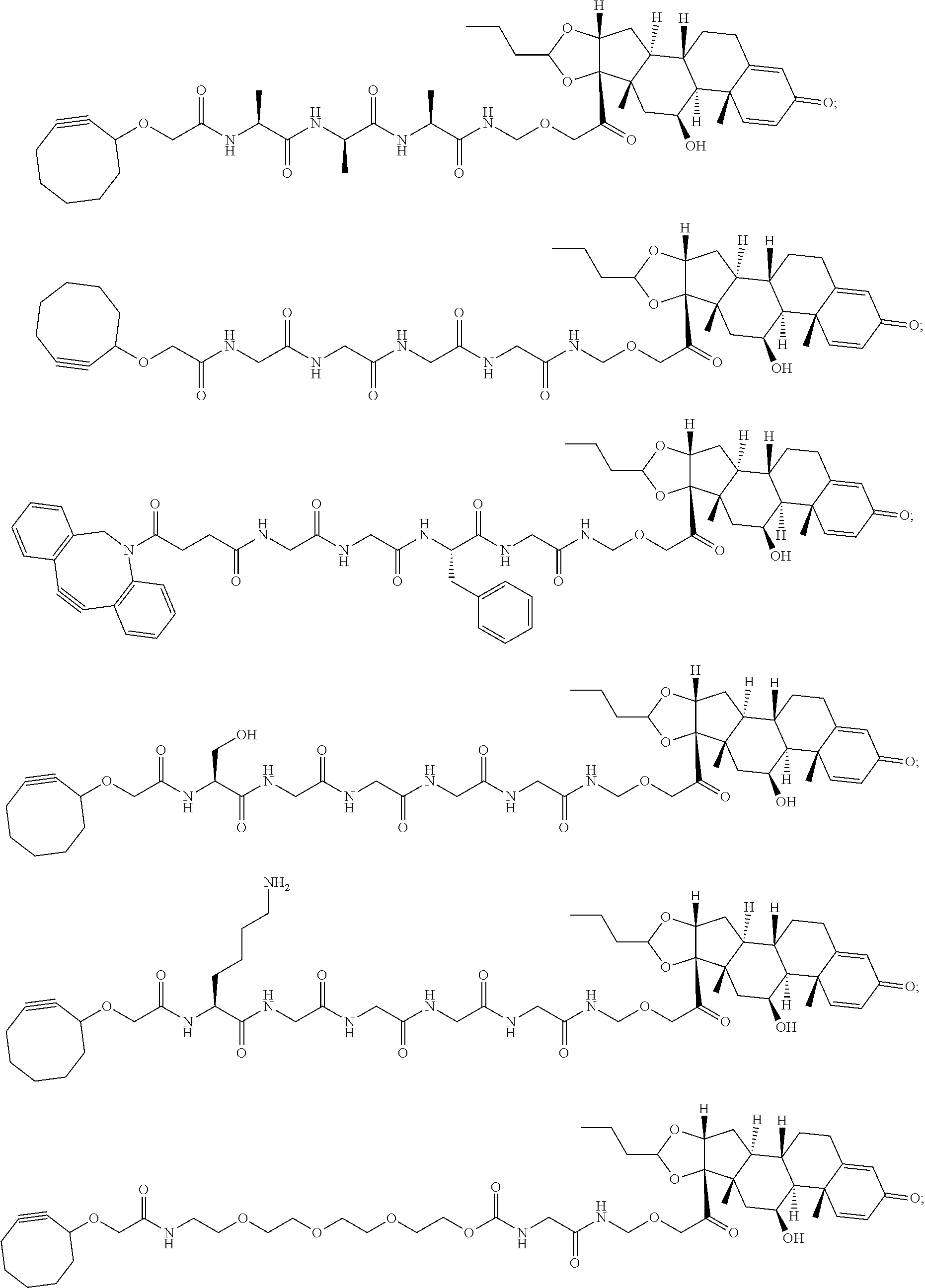

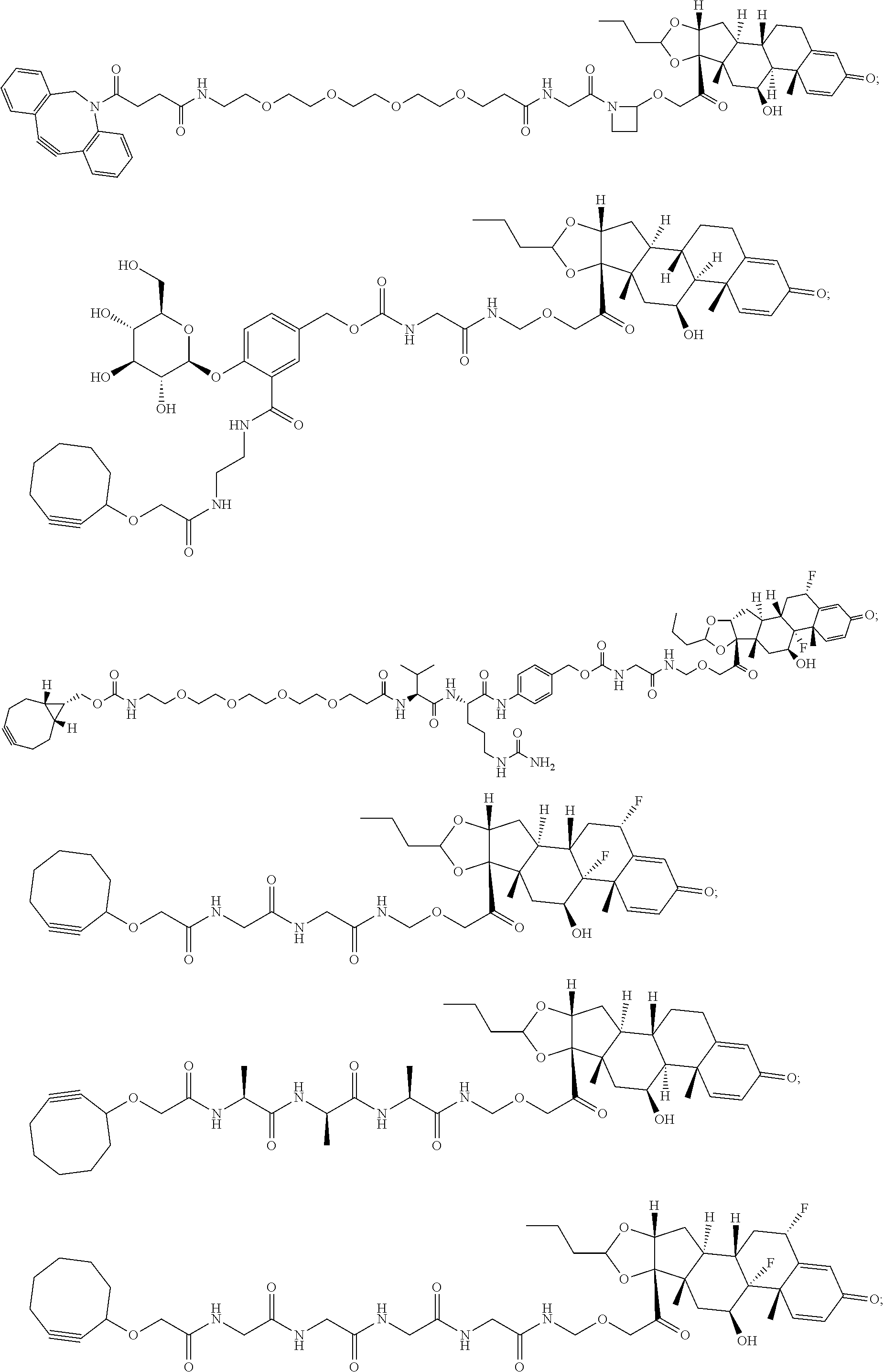

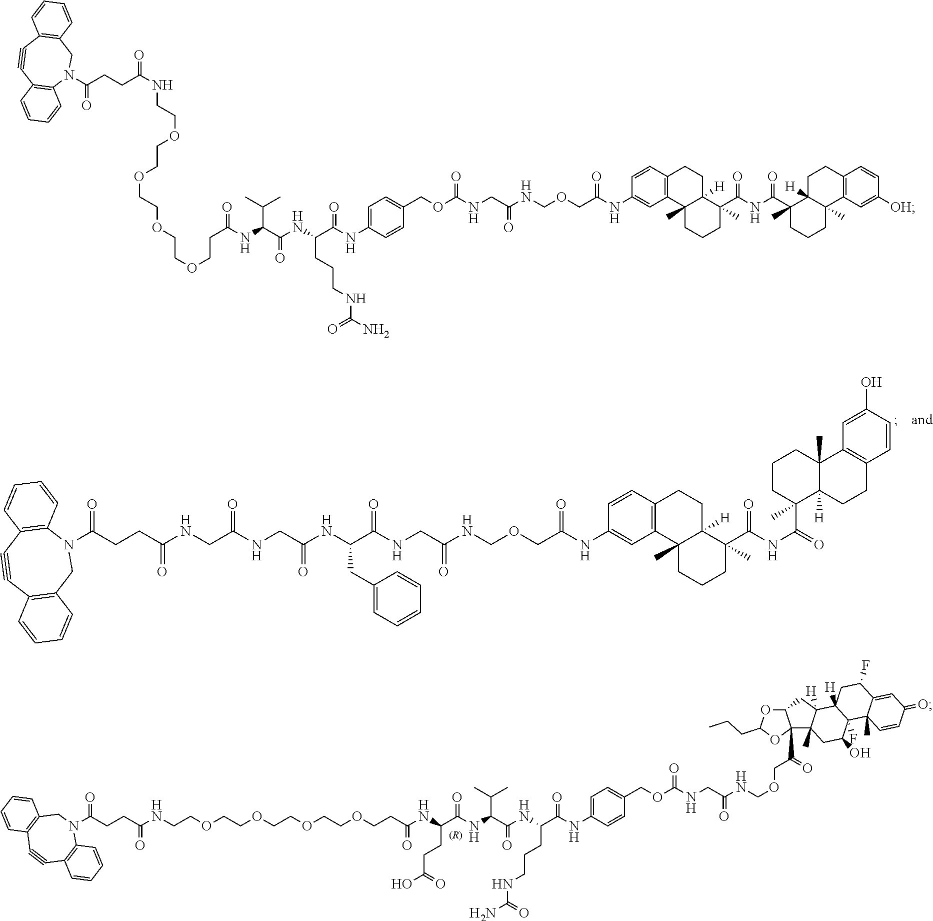

or a pharmaceutically acceptable salt thereof. In certain embodiments within this paragraph, all diastereomers are contemplated. For example, in one embodiment, the stereochemistry at the acetal is undefined or racemic. By way of further example, in one embodiment, the stereochemistry at the acetal is (R)-. By way of further example, in one embodiment, the stereochemistry at the acetal is (S)-. By way of further example, in one embodiment, the stereochemistry at the acetal is (R)- in excess of (S)-. By way of further example, in one embodiment, the stereochemistry at the acetal is (S)- in excess of (R)-.

In certain embodiments of Formula IV or Formula IVa, R^{1a} and R^{1b} are hydrogen; R² is hydrogen; R³ is alkyl; and n is one. In another embodiment of Formula IV or Formula IVa, D* is hydrogen. In another embodiment of Formula IV or Formula IVa, D* is a residue of a biologically active compound comprising hydroxyl. In another embodiment of Formula IV or Formula IVa, the linker-payload has a structure

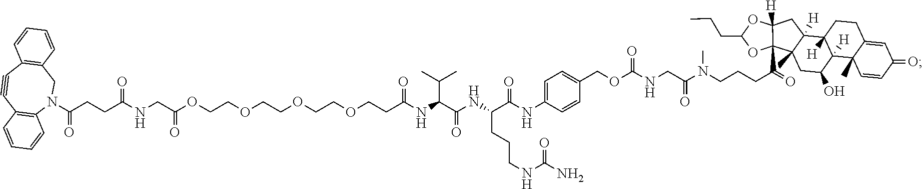

or a pharmaceutically acceptable salt thereof. In certain embodiments within this paragraph, all diastereomers are contemplated. For example, in one embodiment, the stereochemistry at the acetal is undefined or racemic. By way of further example, in one embodiment, the stereochemistry at the acetal is (R)-. By way of further example, in one embodiment, the stereochemistry at the acetal is (S)-. By way of further example, in one embodiment, the stereochemistry at the acetal is (R)- in excess of (S)-. By way of further example, in one embodiment, the stereochemistry at the acetal is (S)- in excess of (R)-.

In certain embodiments of Formula IV or Formula IVa, R^{1a} is alkyl or arylalkyl; R^{1b} is hydrogen; R² is hydrogen; R³ is hydrogen; and n is one. In another embodiment of Formula IV or Formula IVa, D* is hydrogen. In another embodiment of Formula IV or Formula IVa, D* is a residue of a biologically active compound comprising hydroxyl. In another embodiment of Formula IV or Formula IVa, the linker-payload is selected from the group consisting of

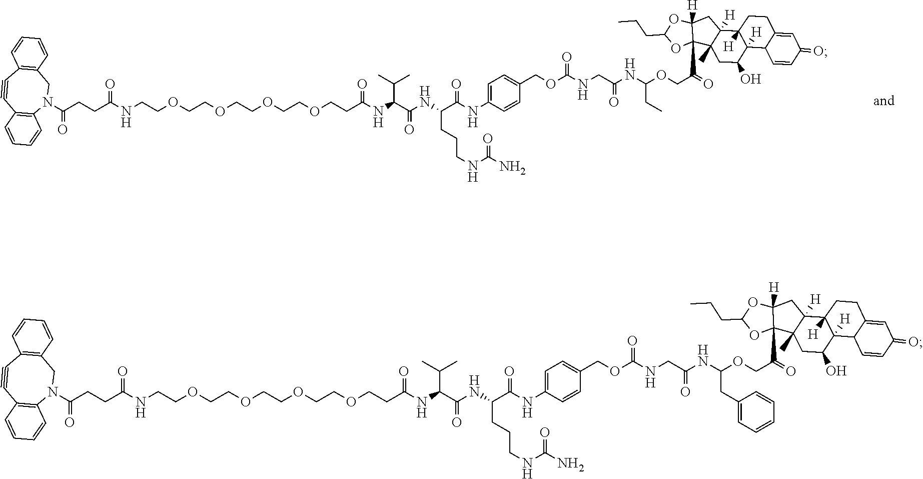

or a pharmaceutically acceptable salt thereof. In certain embodiments within this paragraph, all diastereomers are contemplated. For example, in one embodiment, the stereochemistry at the hemiaminal ether (or hemiaminal, or N-acyl-N,O-acetal, wherein each name for this functional group is used interchangeably throughout this disclosure) is undefined or racemic. By way of further example, in one embodiment, the stereochemistry at the hemiaminal ether is (R)-. By way of further example, in one embodiment, the stereochemistry at the hemiaminal ether is (S)-. By way of further example, in one embodiment, the stereochemistry at the hemiaminal ether is (R)- in excess of (S)-. By way of further example, in one embodiment, the stereochemistry at the hemiaminal ether is (S)- in excess of (R)-. For example, in one embodiment, the stereochemistry at the acetal is undefined or racemic. By way of further example, in one embodiment, the stereochemistry at the acetal is (R)-. By way of further example, in one embodiment, the stereochemistry at the acetal is (S)-. By way of further example, in one embodiment, the stereochemistry at the acetal is (R)- in excess of (S)-. By way of further example, in one embodiment, the stereochemistry at the acetal is (S)- in excess of (R)-.

In certain embodiments of Formula IV or Formula IVa, R^{1a} is alkylene, where the alkylene is further bonded to R³ to form a 4-, 5-, or 6-membered heterocyclyl; R^{1b} is hydrogen; R² is hydrogen; R³ is alkylene further bonded to R^{1a} to form the 4-, 5-, or 6-membered heterocyclyl; and n is one. In another embodiment of Formula IV or Formula IVa, D* is hydrogen. In another embodiment of Formula IV or Formula IVa, D* is a residue of a biologically active compound comprising hydroxyl. In another embodiment of Formula IV or Formula IVa, the linker-payload is selected from the group consisting of

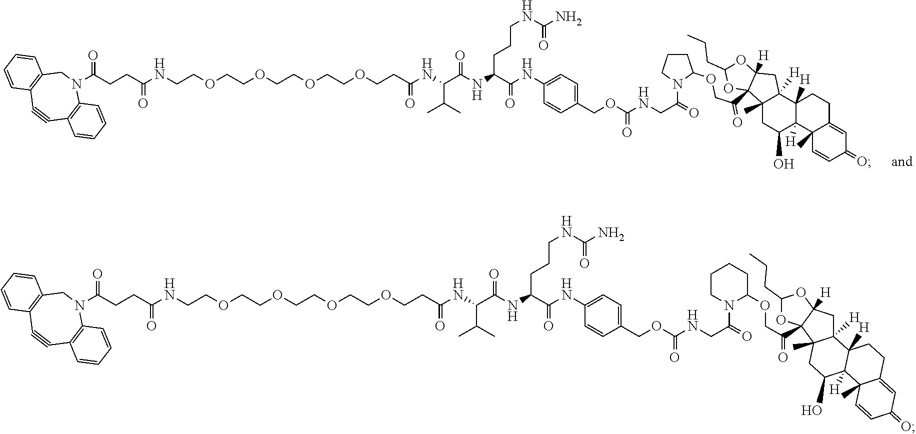 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)O1 ; "and" ; CCCC1O[C@@H]2C[C@H]3[C@@H]4CCC5=CC(=O)C=C[C@]5(C)[C@H]4[C@@H](O)C[C@]3(C)[C@]2(C(=O)COC2CCCCN2C(=O)CNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)O1]

or a pharmaceutically acceptable salt thereof. In another embodiment of Formula IV or Formula IVa, the linker-payload has a structure

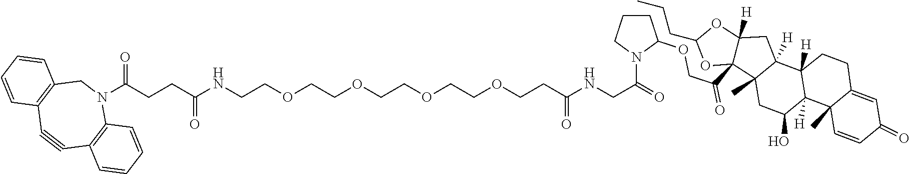 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CNC(=O)CCOCCOCCOCCOCCNC(=O)CCC(=O)N2Cc3ccccc3C#Cc3ccccc32)O1]

or a pharmaceutically acceptable salt thereof. In certain embodiments within this paragraph, all diastereomers are contemplated. For example, in one embodiment, the stereochemistry at the hemiaminal ether (or hemiaminal, or N-acyl-N,O-acetal, wherein each name for this functional group is used interchangeably throughout this disclosure) is undefined or racemic. By way of further example, in one embodiment, the stereochemistry at the hemiaminal ether is (R)-. By way of further example, in one embodiment, the stereochemistry at the hemiaminal ether is (S)-. By way of further example, in one embodiment, the stereochemistry at the hemiaminal ether is (R)- in excess of (S)-. By way of further example, in one embodiment, the stereochemistry at the hemiaminal ether is (S)- in excess of (R)-. For example, in one embodiment, the stereochemistry at the acetal is undefined or racemic. By way of further example, in one embodiment, the stereochemistry at the acetal is (R)-. By way of further example, in one embodiment, the stereochemistry at the acetal is (S)-. By way of further example, in one embodiment, the stereochemistry at the acetal is (R)- in excess of (S)-. By way of further example, in one embodiment, the stereochemistry at the acetal is (S)- in excess of (R)-.

In certain embodiments of Formula IV or Formula IVa, R^{1a} and R^{1b} are hydrogen; R² is hydrogen; R³ is hydrogen; and n is one. In another embodiment of Formula IV or Formula IVa, D* is hydrogen. In another embodiment of Formula IV or Formula IVa, D* is a residue of a biologically active compound comprising hydroxyl. In another embodiment of Formula IV or Formula IVa, the linker-payload has the following structure

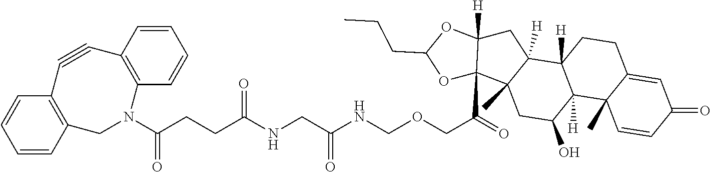 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CCC(=O)N2Cc3ccccc3C#Cc3ccccc32)O1]

or a pharmaceutically acceptable salt thereof. In certain embodiments within this paragraph, all diastereomers are contemplated.

In certain embodiments of Formula IV or Formula IVa, R^{1a} and R^{1b} are hydrogen; R² is hydrogen or —CH₂Ph; R³ is hydrogen; and n is four. In another embodiment of Formula IV or Formula IVa, D* is hydrogen. In another embodiment of Formula IV or Formula IVa, D* is a residue of a biologically active compound comprising hydroxyl. In another embodiment of Formula IV or Formula IVa, the linker-payload is selected from the group consisting of

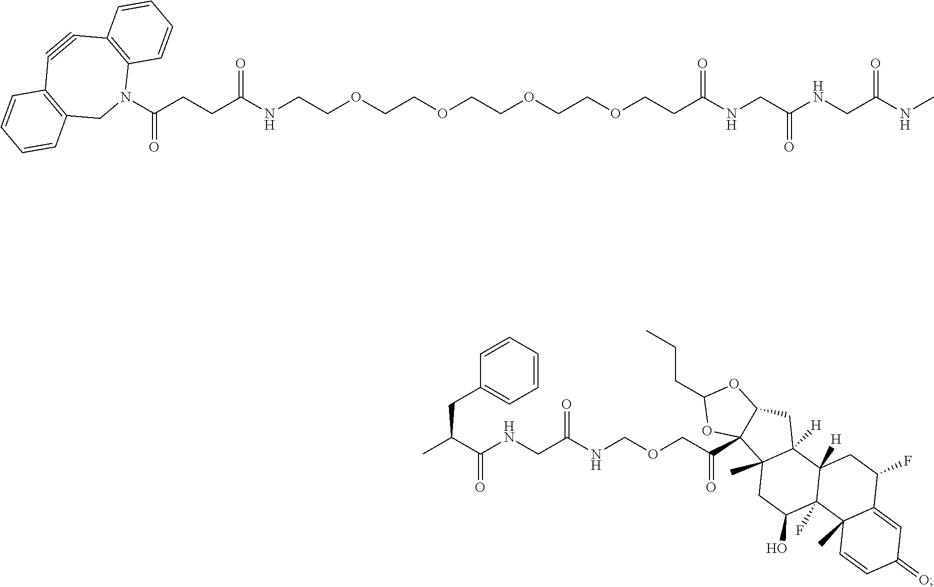

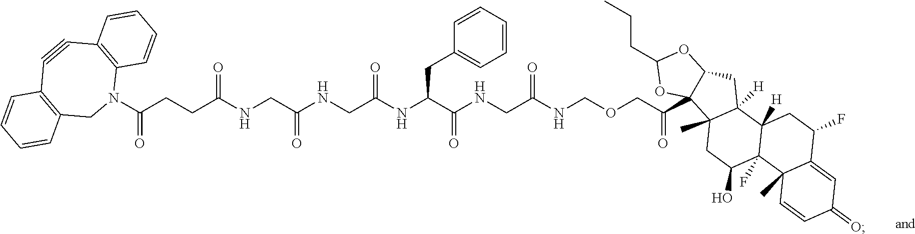

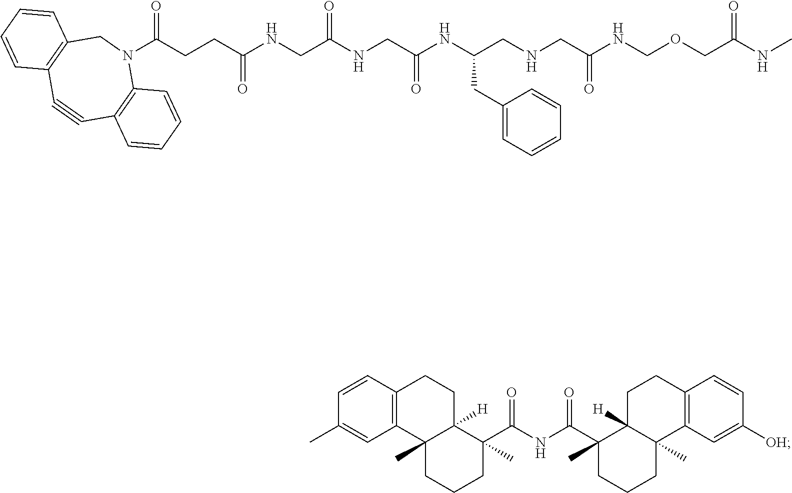

or a pharmaceutically acceptable salt thereof. In certain embodiments within this paragraph, all diastereomers are contemplated. For example, in one embodiment, the stereochemistry at the acetal is undefined or racemic. By way of further example, in one embodiment, the stereochemistry at the acetal is (R)-. By way of further example, in one embodiment, the stereochemistry at the acetal is (S)-. By way of further example, in one embodiment, the stereochemistry at the acetal is (R)- in excess of (S)-. By way of further example, in one embodiment, the stereochemistry at the acetal is (S)- in excess of (R)-.

In certain embodiments, of Formula IV or Formula IVa, R^{1a} and R^{1b} are hydrogen; R² is hydrogen or —CH₂OH; R³ is hydrogen; and n is six. In another embodiment of Formula IV or Formula IVa, D* is hydrogen. In another embodiment of Formula IV or Formula IVa, D* is a residue of a biologically active compound comprising hydroxyl. In another embodiment of Formula IV or Formula IVa, the linker-payload has the following structure

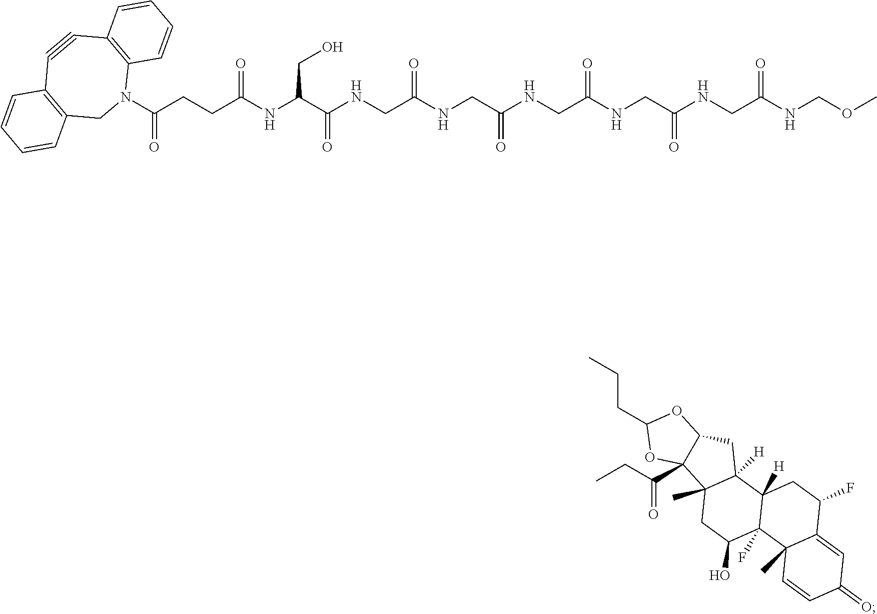

or a pharmaceutically acceptable salt thereof. In certain embodiments within this paragraph, all diastereomers are contemplated.

In certain embodiments of Formula IV or Formula IVa, R^{1a} and R^{1b} are hydrogen; R² is alkylene, wherein the alkylene is further bonded to R³ to form a 6-membered heterocyclyl; R³ is alkylene, wherein the alkylene is further bonded to R² to form the 6-membered heterocyclyl; and n is one. In another embodiment of Formula IV or Formula IVa, D* is hydrogen. In another embodiment of Formula IV or Formula IVa, D* is a residue of a biologically active compound comprising hydroxyl. In another embodiment of Formula IV or Formula IVa, the linker-payload has the following structure

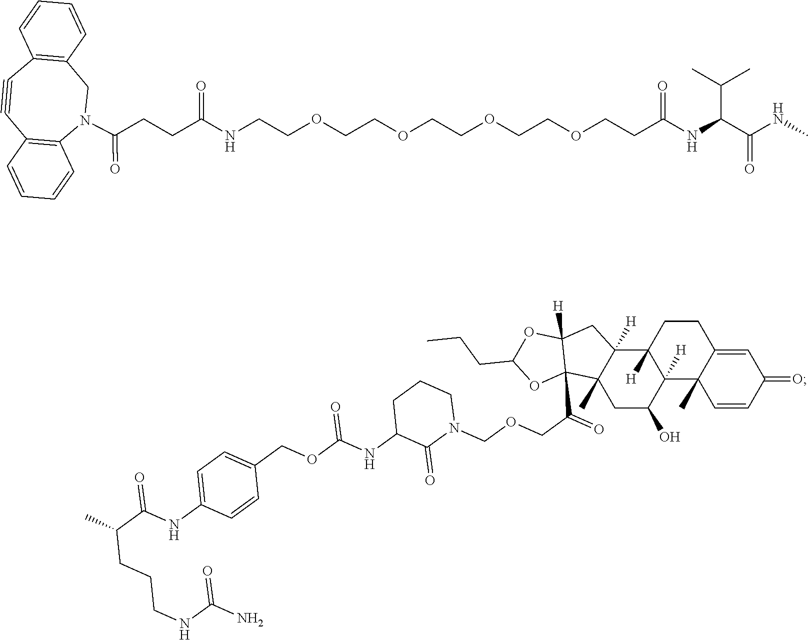

or a pharmaceutically acceptable salt thereof.

In certain embodiments, other linker-payloads are contemplated. In certain embodiments of Formula IIb, R^{1a} and R^{1b} are hydrogen; R³ is hydrogen; and n is zero. In another embodiment of Formula IIb, D* is hydrogen. In another embodiment of Formula IIb, D* is a residue of a biologically active compound comprising hydroxyl. In certain embodiments of Formula IV or Formula IVa, R^{1a} and R^{1b} are hydrogen; R² is hydrogen; R³ is alkyl; R⁴ is alkyl; and n is one. In another embodiment of Formula IV or Formula IVa, D* is hydrogen. In another embodiment of Formula IV or Formula IVa, D* is a residue of a biologically active compound comprising hydroxyl. Exemplary linker-payloads contemplated include, without limitation,

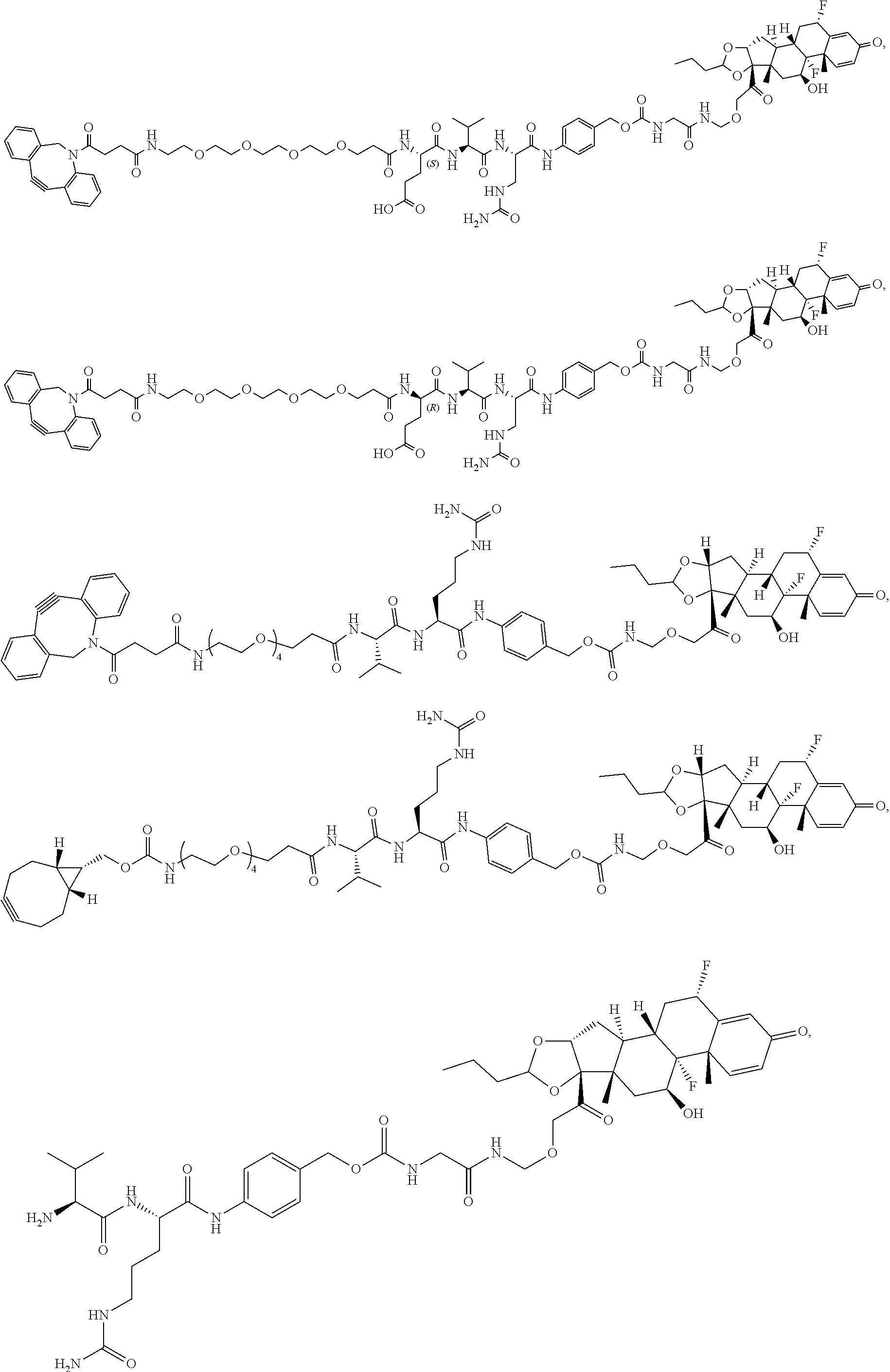

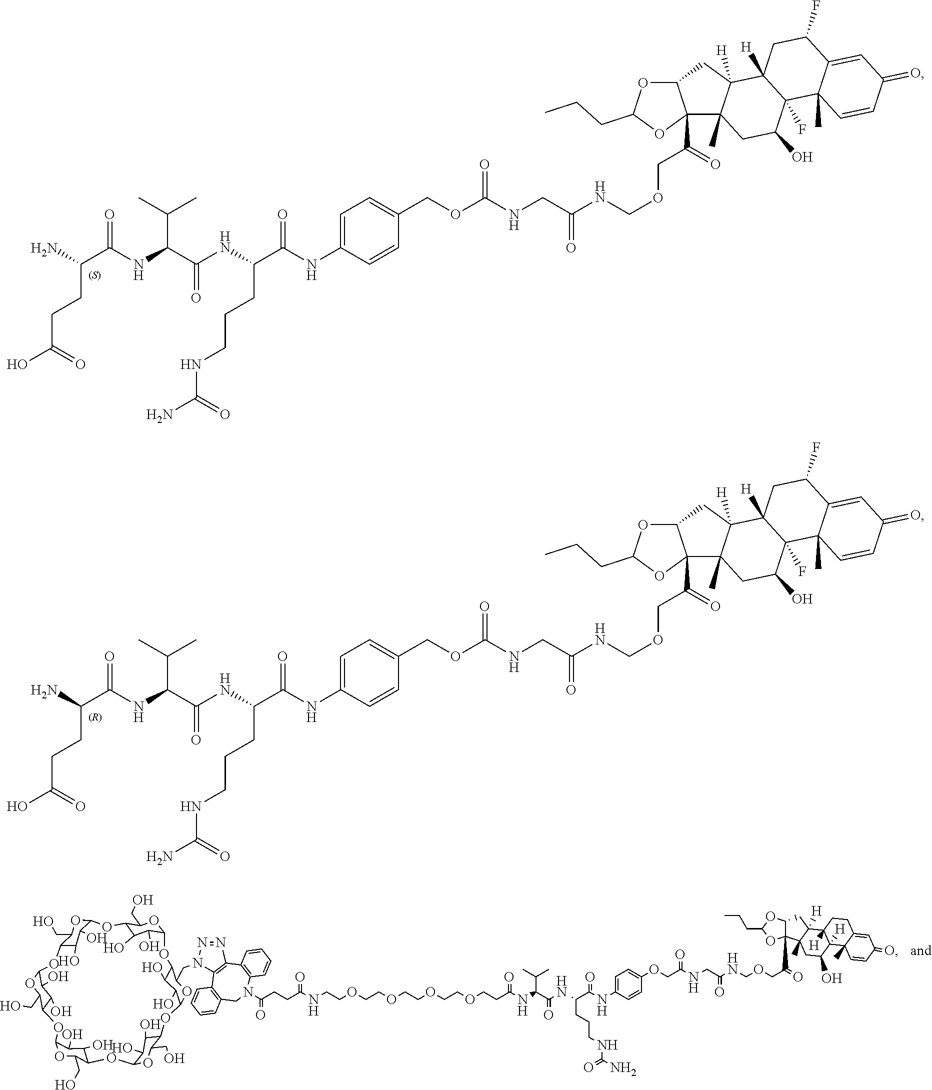

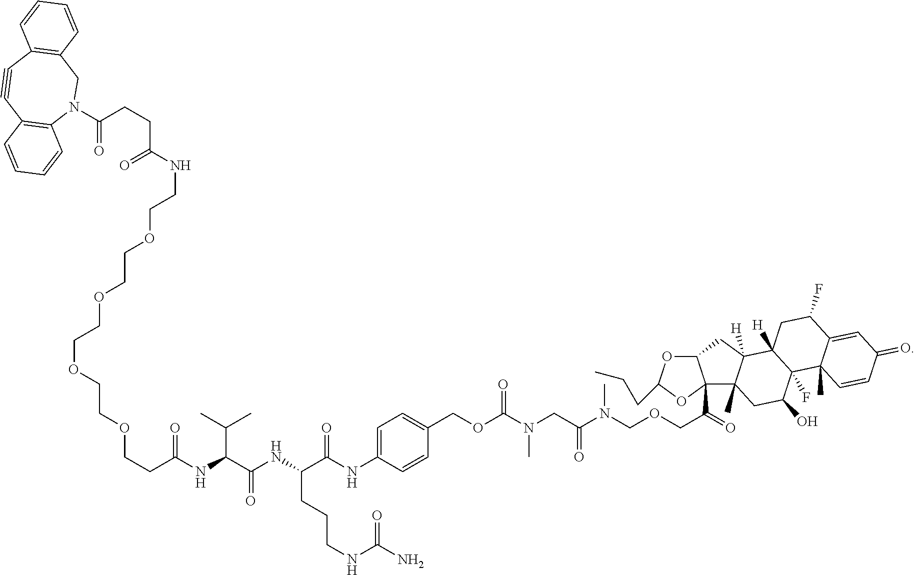 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCN(C)C(=O)CN(C)C(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)O1]

### Conjugates/Antibody-Drug Conjugates (ADCs)

Provided herein are antibodies, or an antigen binding fragment thereof, wherein said antibody is conjugated to one or more compounds of Formula I, Ia, Iaa, Iaaa, Ib, Ibb, Ibbb, II, IIa, Iaa, Iaaa, IIb, IIbb, IIbbb, and/or IV as described herein. In one embodiment of Formula III, D* is a residue of an anti-inflammatory biologically active compound comprising hydroxyl, amino, or thiol. In another embodiment of Formula III, the anti-inflammatory biologically active compound is a steroid or a residue thereof. In another embodiment of Formula III, the anti-inflammatory biologically active compound is an LXR agonist or a residue thereof.

Provided herein are conjugates of Formula IIIa:

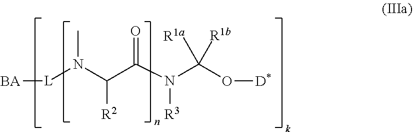

wherein L is a linker; BA is a binding agent; R^{1a} and R^{1b} are, independently, hydrogen, alkyl, alkoxy, alkenyl, alkynyl, aryl, arylalkyl, heteroaryl, or alkylene, wherein when R^{1a} is alkylene, the alkylene is further bonded to R³ to form a 4-, 5-, or 6-membered heterocyclyl; R² is hydrogen or an amino acid side chain; R³ is hydrogen, alkyl, or alkylene, wherein when R³ is alkylene, the alkylene is further bonded to R^{1a} to form the 4-, 5-, or 6-membered heterocyclyl; D* is a residue of a biologically active compound comprising hydroxyl; n is zero, one, two, three, four, five, or six; and k is an integer from one to thirty. In another embodiment, provided is a conjugate of Formula IIIaa:

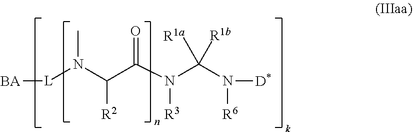

wherein L is a linker; BA is a binding agent; R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; R⁶ is hydrogen or alkyl; D* is a residue of a biologically active compound comprising hydroxyl, amino, or thiol; n is zero, one, two, three, four, or five; and k is an integer from one to thirty. In another embodiment, provided is a conjugate of Formula IIIaaa:

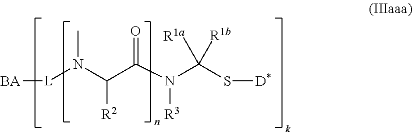

wherein L is a linker; BA is a binding agent; R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; D* is a residue of a biologically active compound comprising hydroxyl, amino, or thiol; n is zero, one, two, three, four, or five; and k is an integer from one to thirty. Also provided herein are conjugates of Formula IIIb:

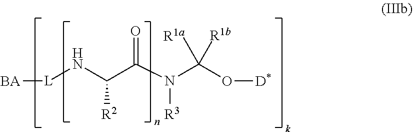

wherein L is a linker; BA is a binding agent; R^{1a} and R^{1b} are, independently, hydrogen, alkyl, alkoxy, alkenyl, alkynyl, aryl, arylalkyl, heteroaryl, or alkylene, wherein when R^{1a} is alkylene, the alkylene is further bonded to R³ to form a 4-, 5-, or 6-membered heterocyclyl; R² is hydrogen or an amino acid side chain; R³ is hydrogen, alkyl, or alkylene, wherein when R³ is alkylene, the alkylene is further bonded to R^{1a} to form the 4-, 5-, or 6-membered heterocyclyl; D* is a residue of a biologically active compound comprising hydroxyl; n is zero, one, two, three, four, five, or six; and k is an integer from one to thirty. In another embodiment, provided is a conjugate of Formula IIIbb:

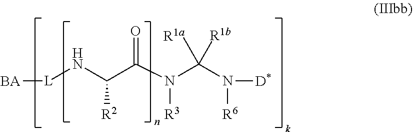

wherein L is a linker; BA is a binding agent; R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; R⁶ is hydrogen or alkyl; D* is a residue of a biologically active compound comprising hydroxyl, amino, or thiol; n is zero, one, two, three, four, or five; and k is an integer from one to thirty. In another embodiment, provided is a conjugate of Formula IIIbbb:

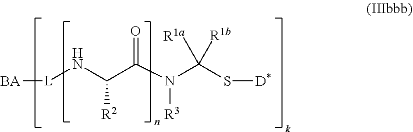

wherein L is a linker; BA is a binding agent; R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl; R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl; R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; D* is a residue of a biologically active compound comprising hydroxyl, amino, or thiol; n is zero, one, two, three, four, or five; and k is an integer from one to thirty. Also provided herein are conjugates of Formula V:

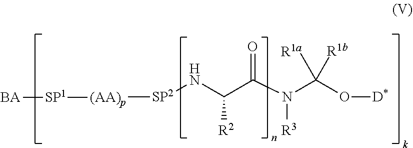

wherein BA, R^{1a}, R^{1b}, R², R³, D*, n, and k are as described in any of the embodiments disclosed herein, and wherein SP¹ and SP², when present, are spacer groups wherein SP¹ further comprises a moiety reactive with an antibody or an antigen binding fragment thereof; each AA is an amino acid; and p is an integer from 1 to 10. In certain embodiments of Formula V, the binding agent is an antibody modified with a primary amine compound according to the Formula H₂N-LL-X, wherein LL is a divalent linker selected from the group consisting of a divalent polyethylene glycol (PEG) group;
—(CH₂)ₙ—;
—(CH₂CH₂O)ₙ—(CH₂)ₚ—;
—(CH₂)ₙ—N(H)C(O)—(CH₂)ₘ—;
—(CH₂CH₂O)ₙ—N(H)C(O)—(CH₂CH₂O)ₘ—(CH₂)ₚ—;
—(CH₂)ₙ—C(O)N(H)—(CH₂)ₘ—;
—(CH₂CH₂O)ₙ—C(O)N(H)—(CH₂CH₂O)ₘ—(CH₂)ₚ—;
—(CH₂)ₙ—N(H)C(O)—(CH₂CH₂O)ₘ—(CH₂)ₚ—;
—(CH₂CH₂O)ₙ—N(H)C(O)—(CH₂)ₘ—;
—(CH₂)ₙ—C(O)N(H)—(CH₂CH₂O)ₘ—(CH₂)ₚ—; and
—(CH₂CH₂O)ₙ—C(O)N(H)—(CH₂)ₘ—,
wherein
n is an integer selected from 1 to 12;
m is an integer selected from 0 to 12;
p is an integer selected from 0 to 2; and
X is selected from the group consisting of —SH, —N₃, —C≡CH, —C(O)H, tetrazole,

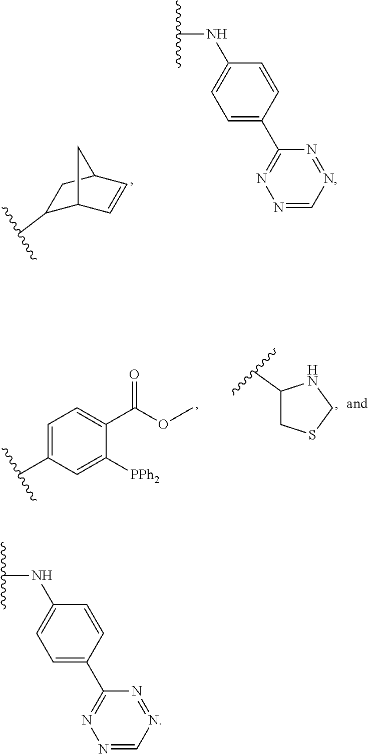

In another embodiment of Formula V, the binding agent is an antibody modified with a primary amine having the following structure

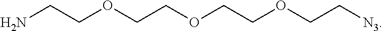 [SMILES: [N-]=[N+]=NCCOCCOCCOCCN]

In another embodiment of Formula V, the compound is selected from the group consisting of

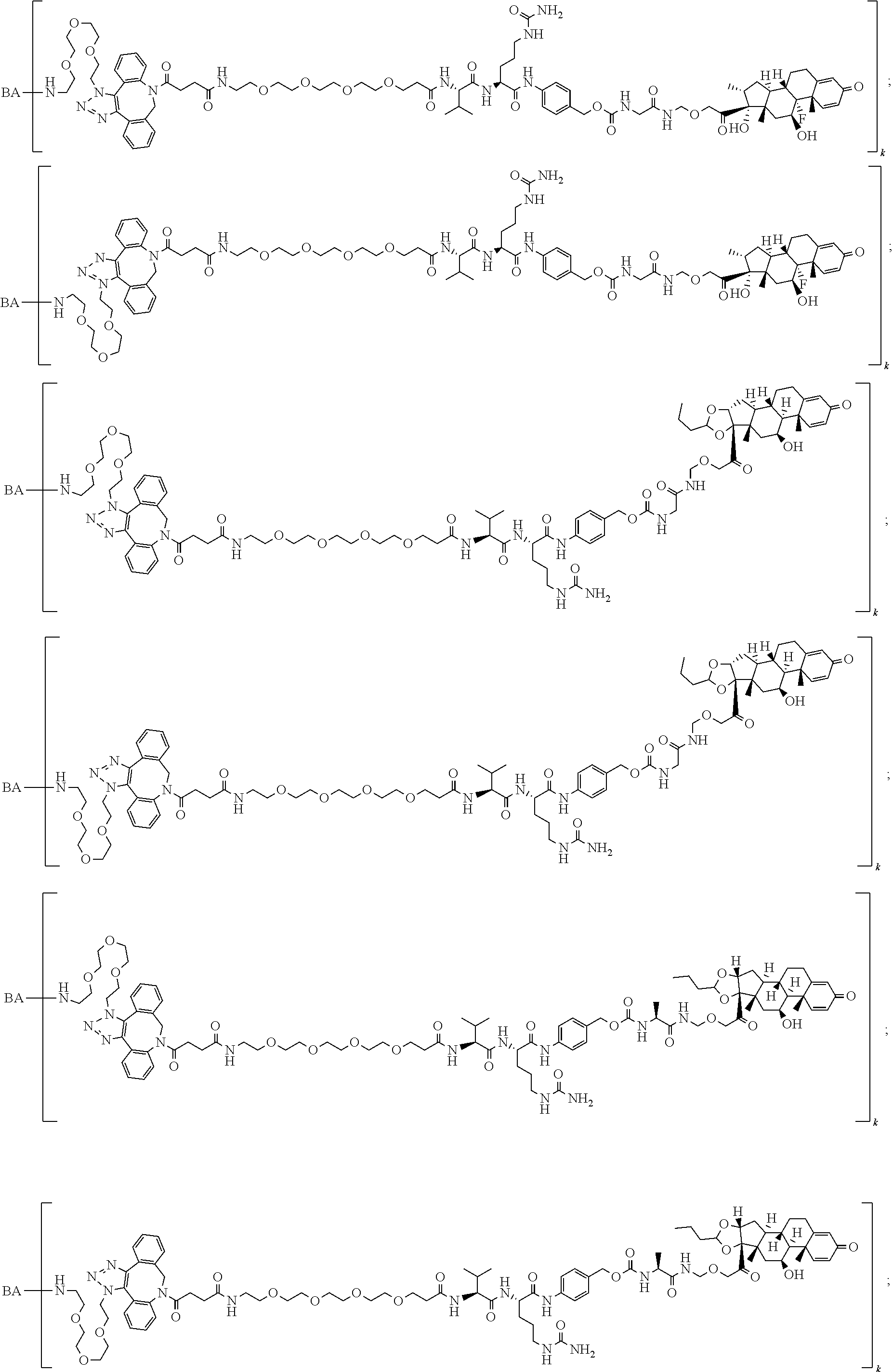

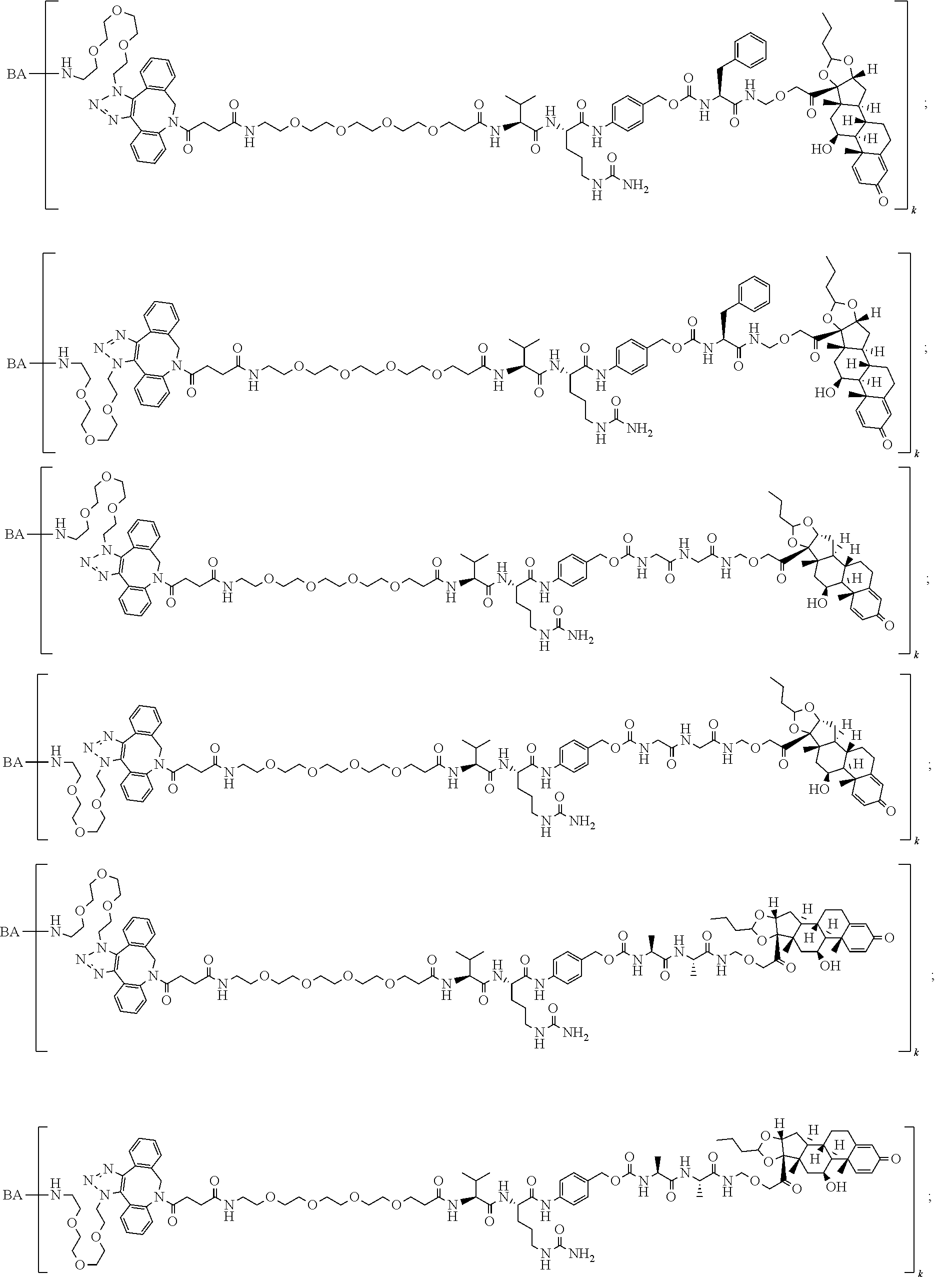

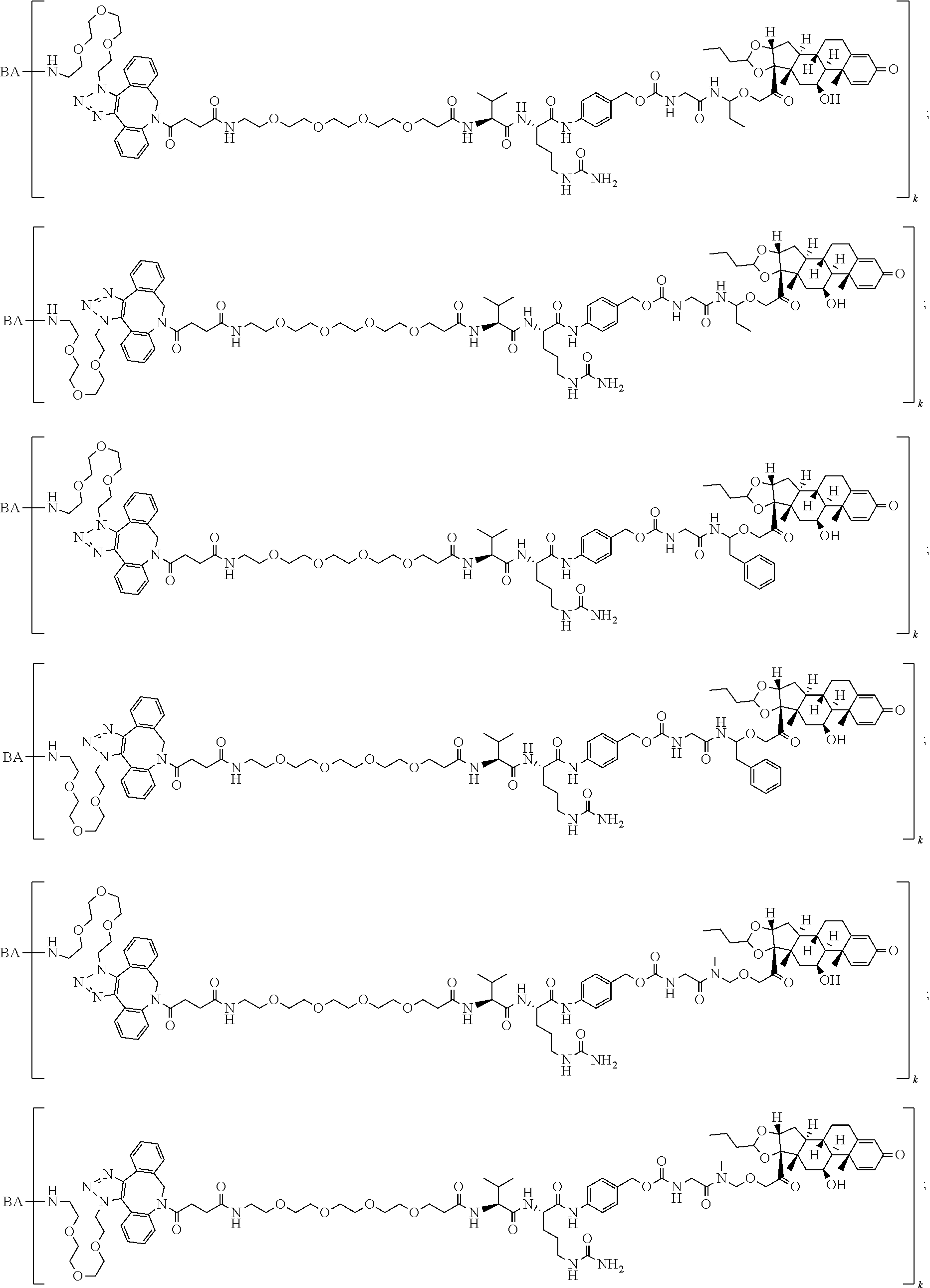

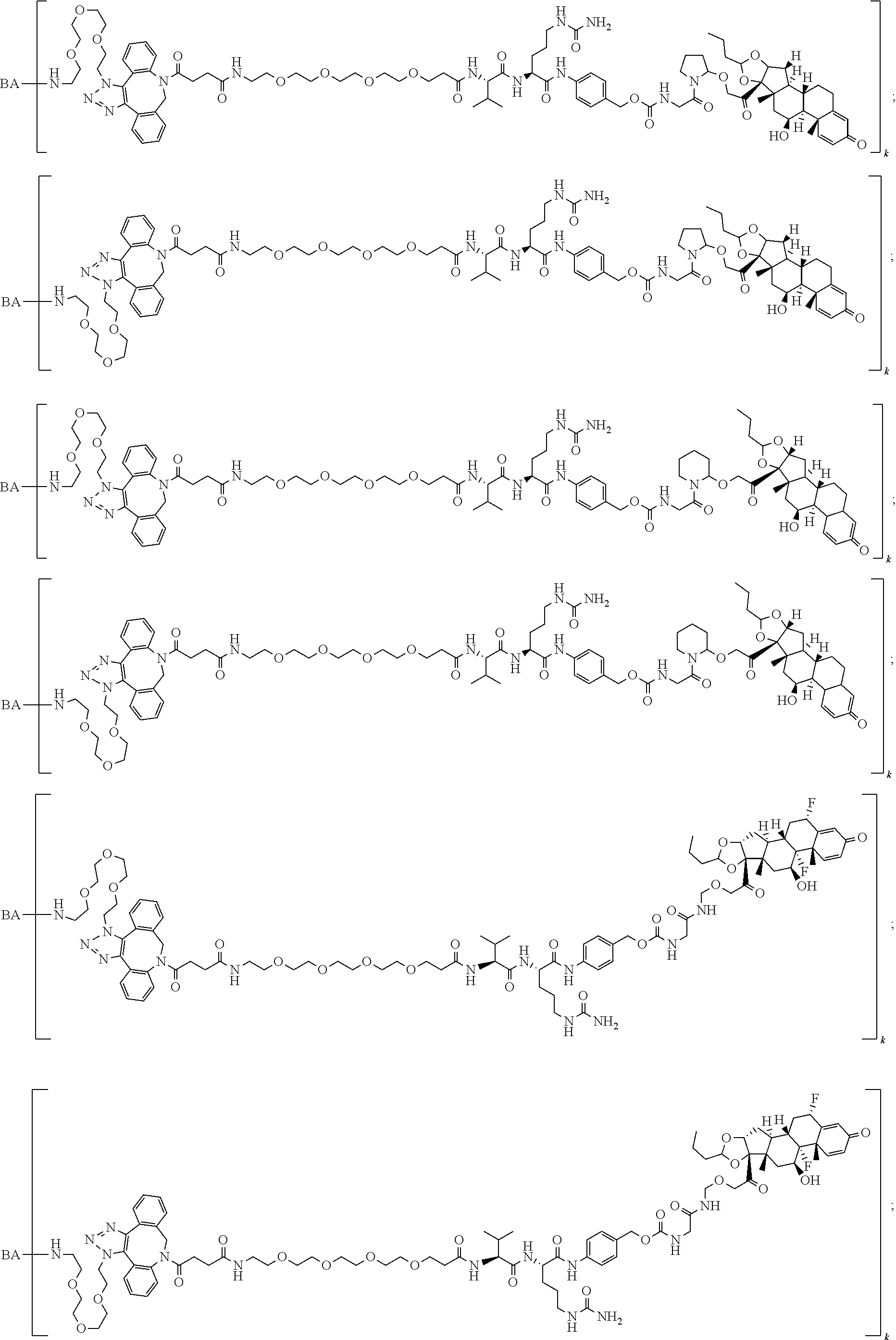

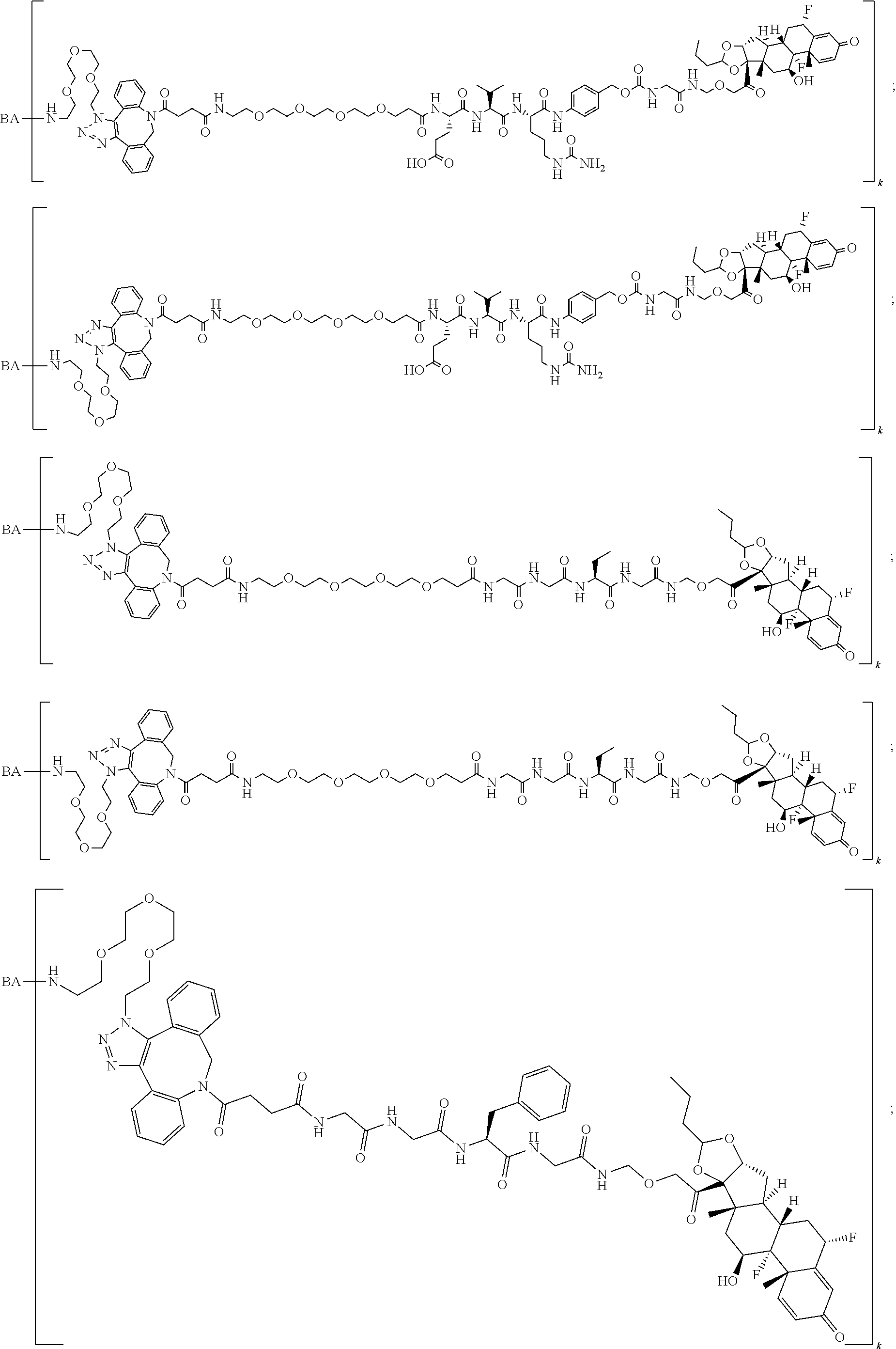

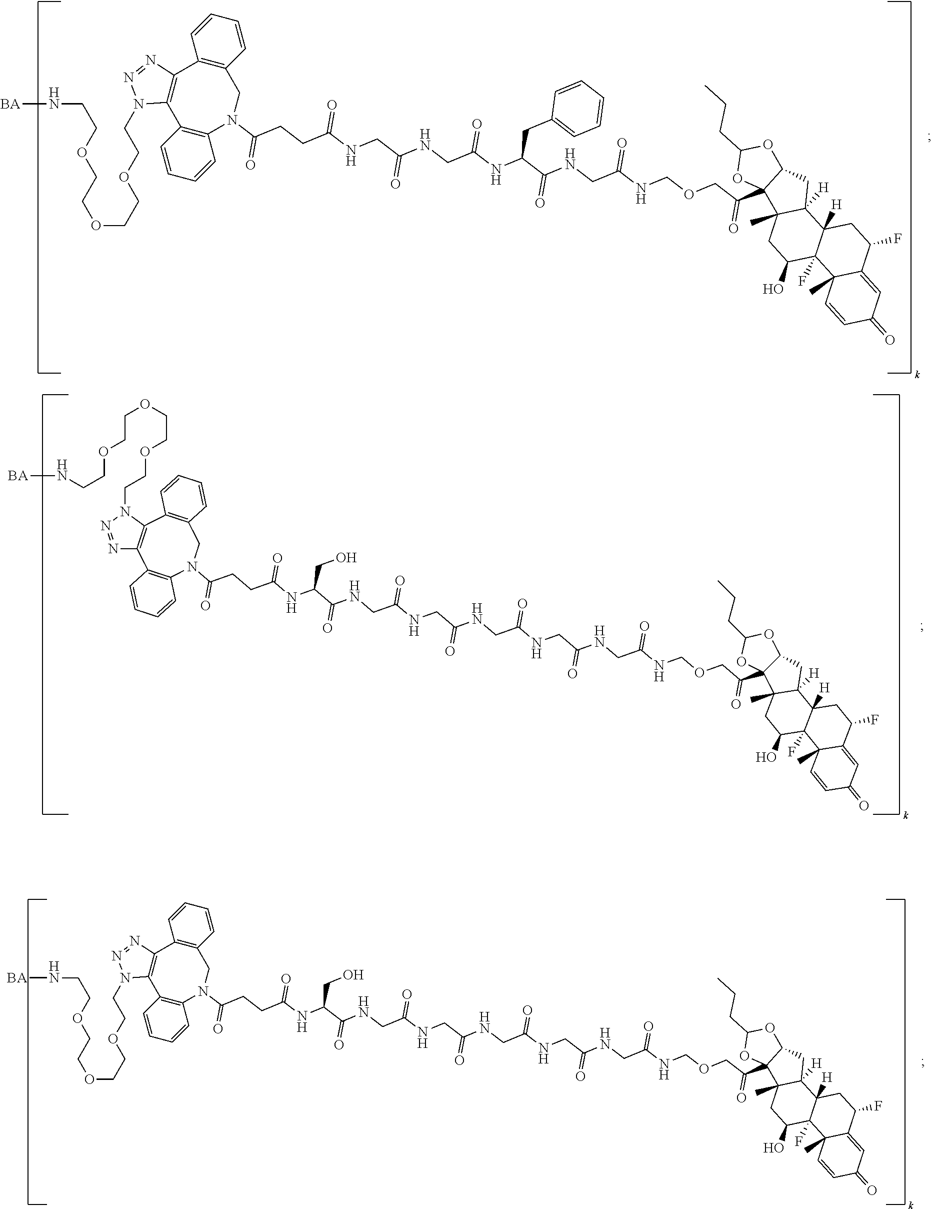

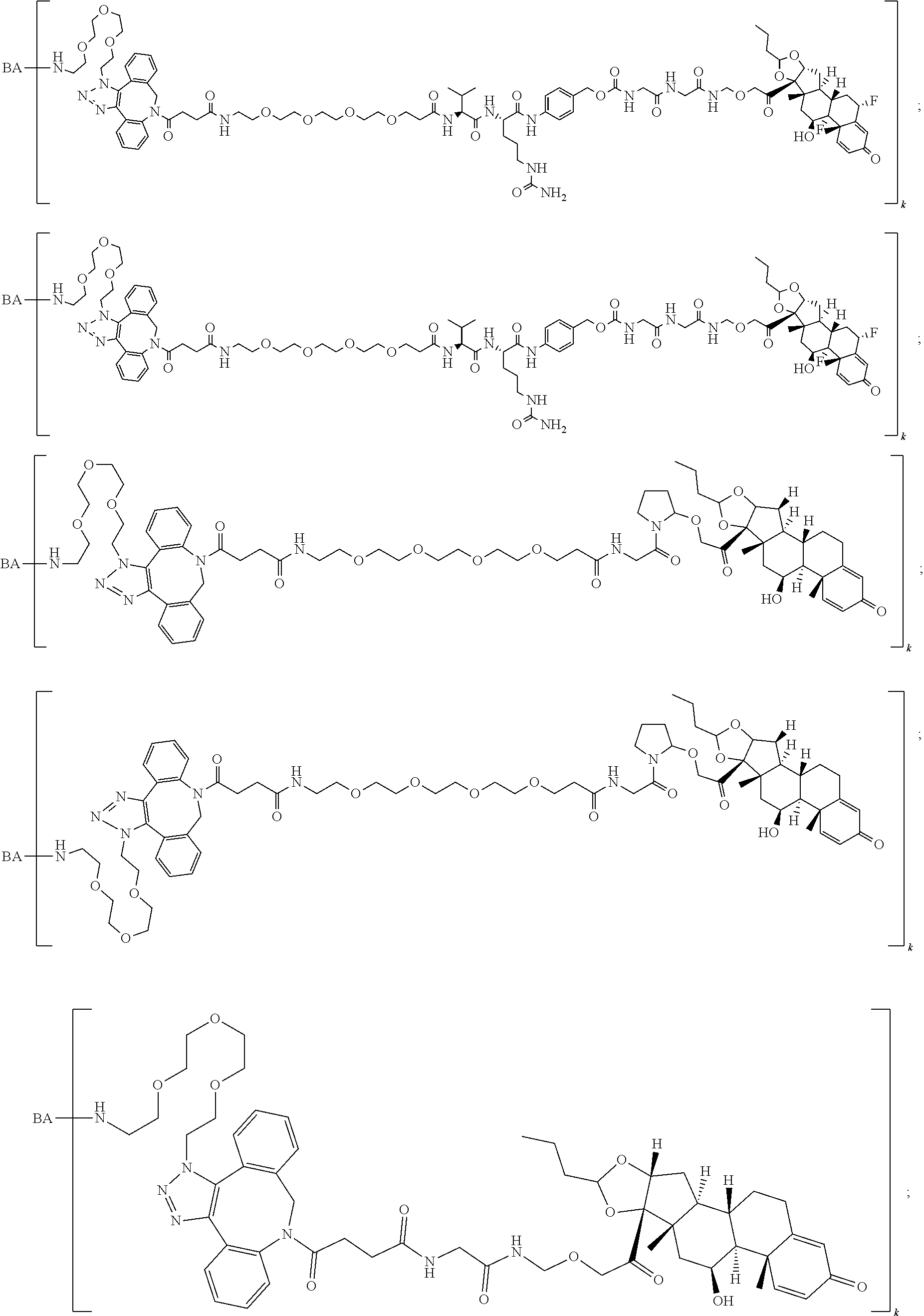

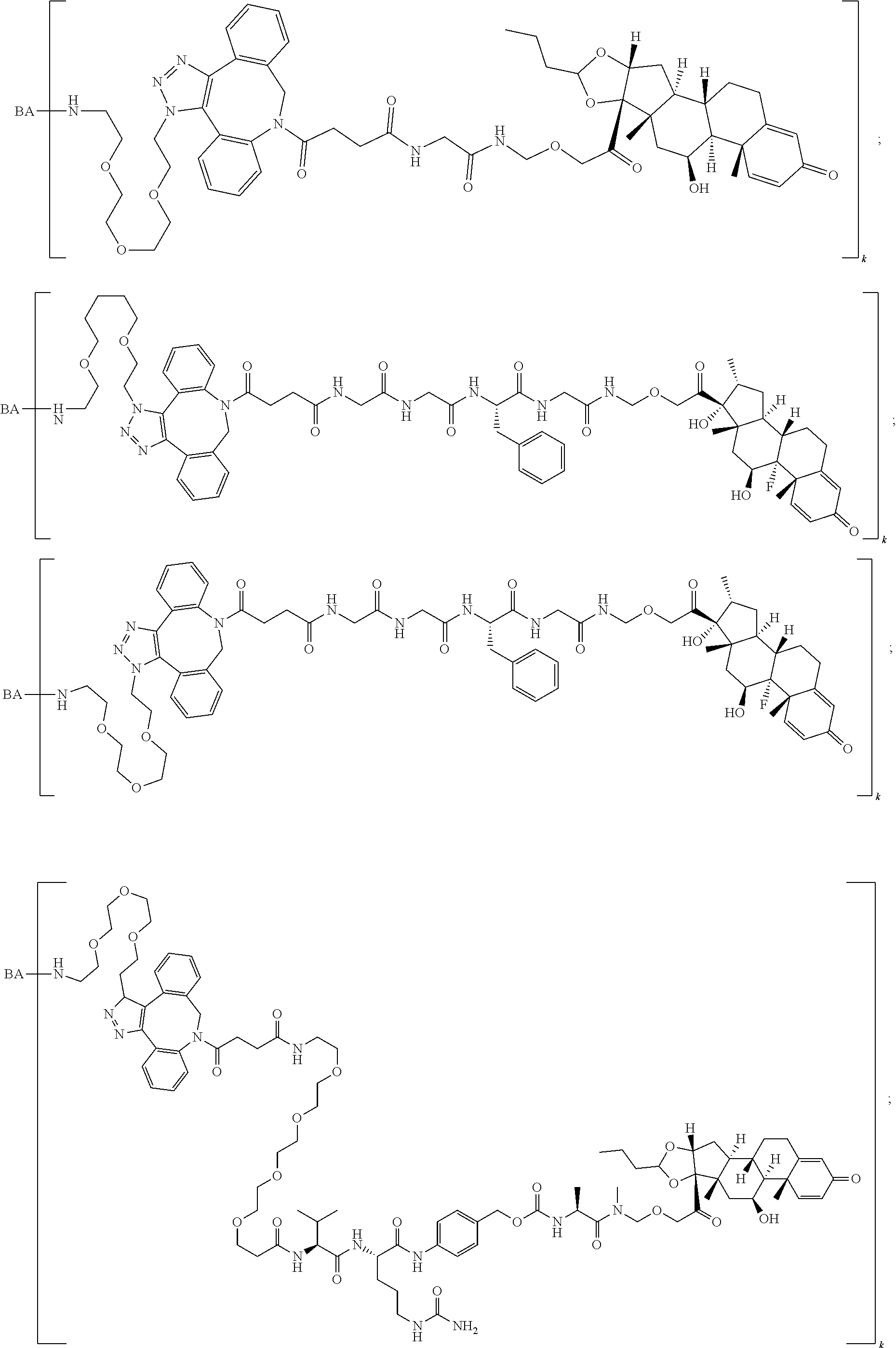

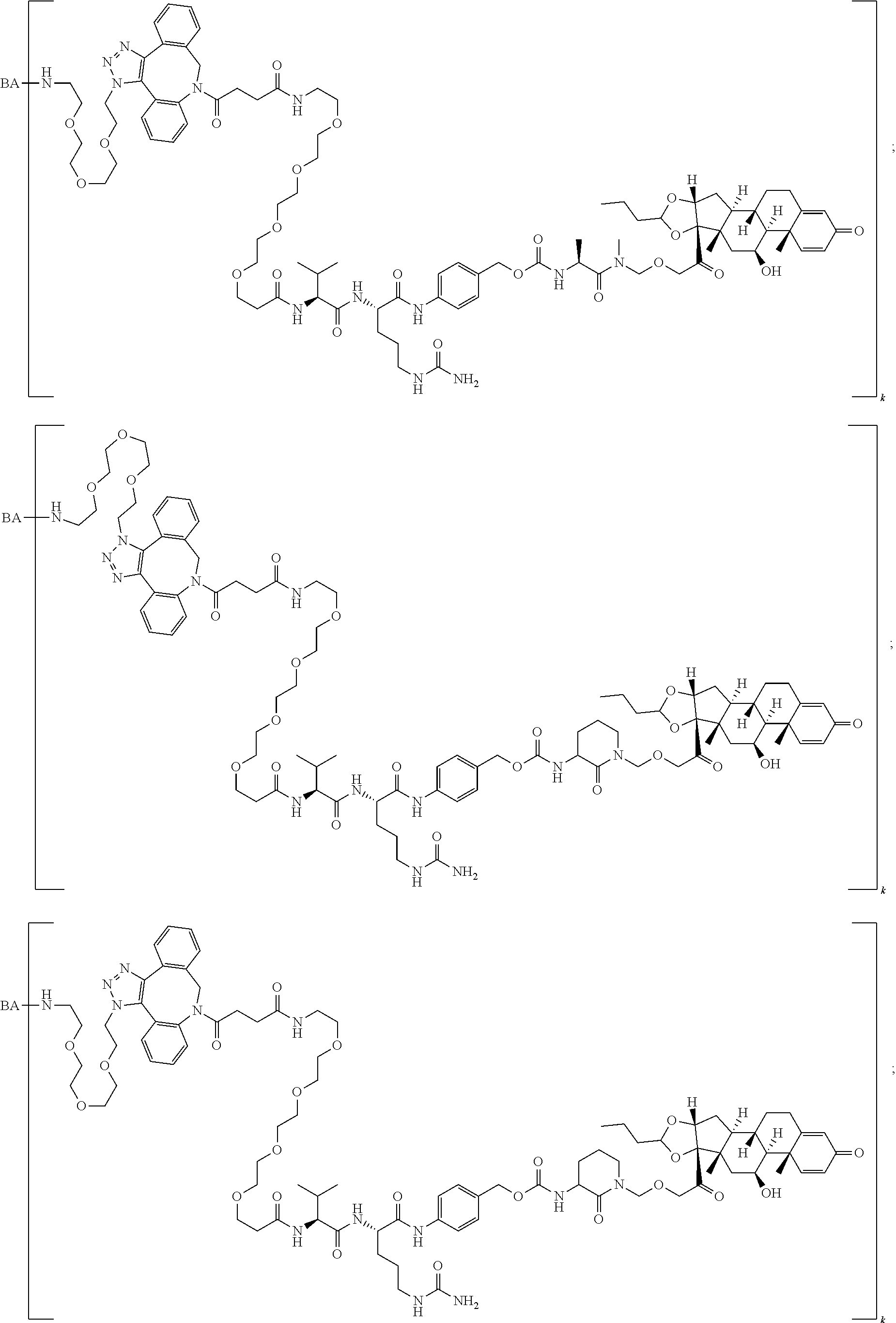

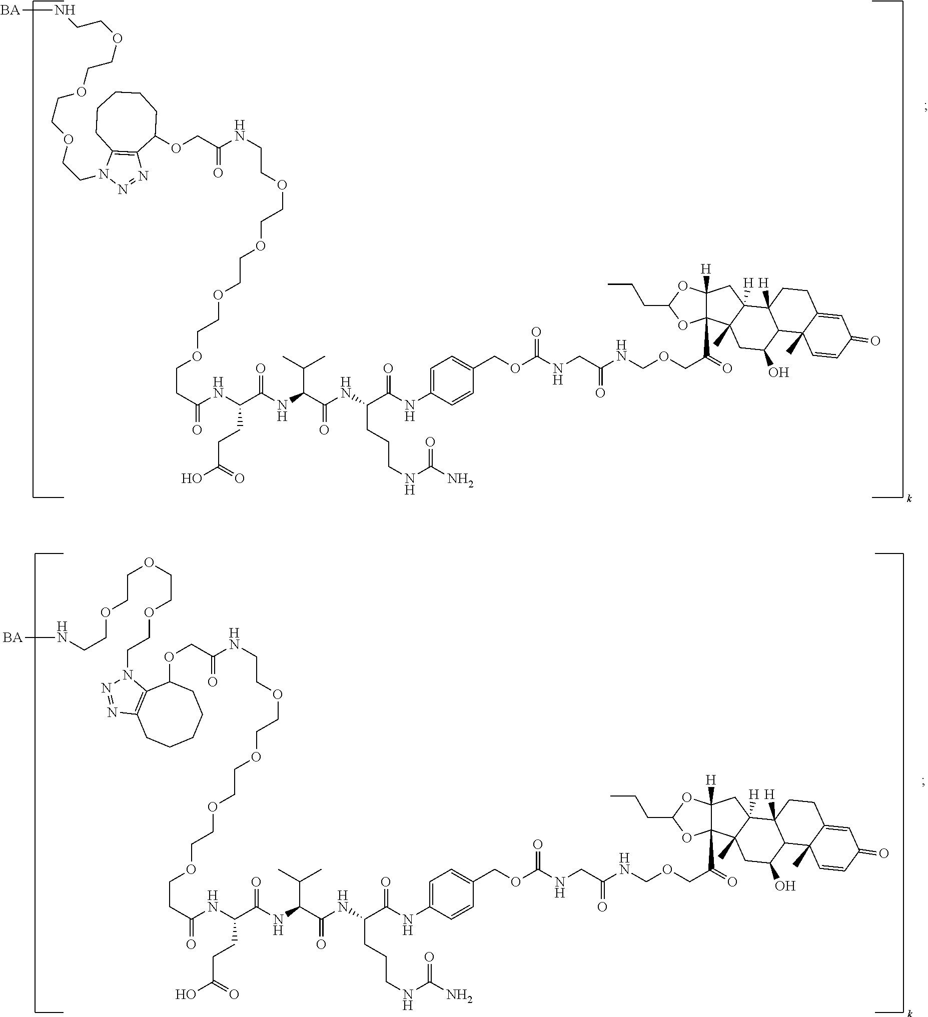

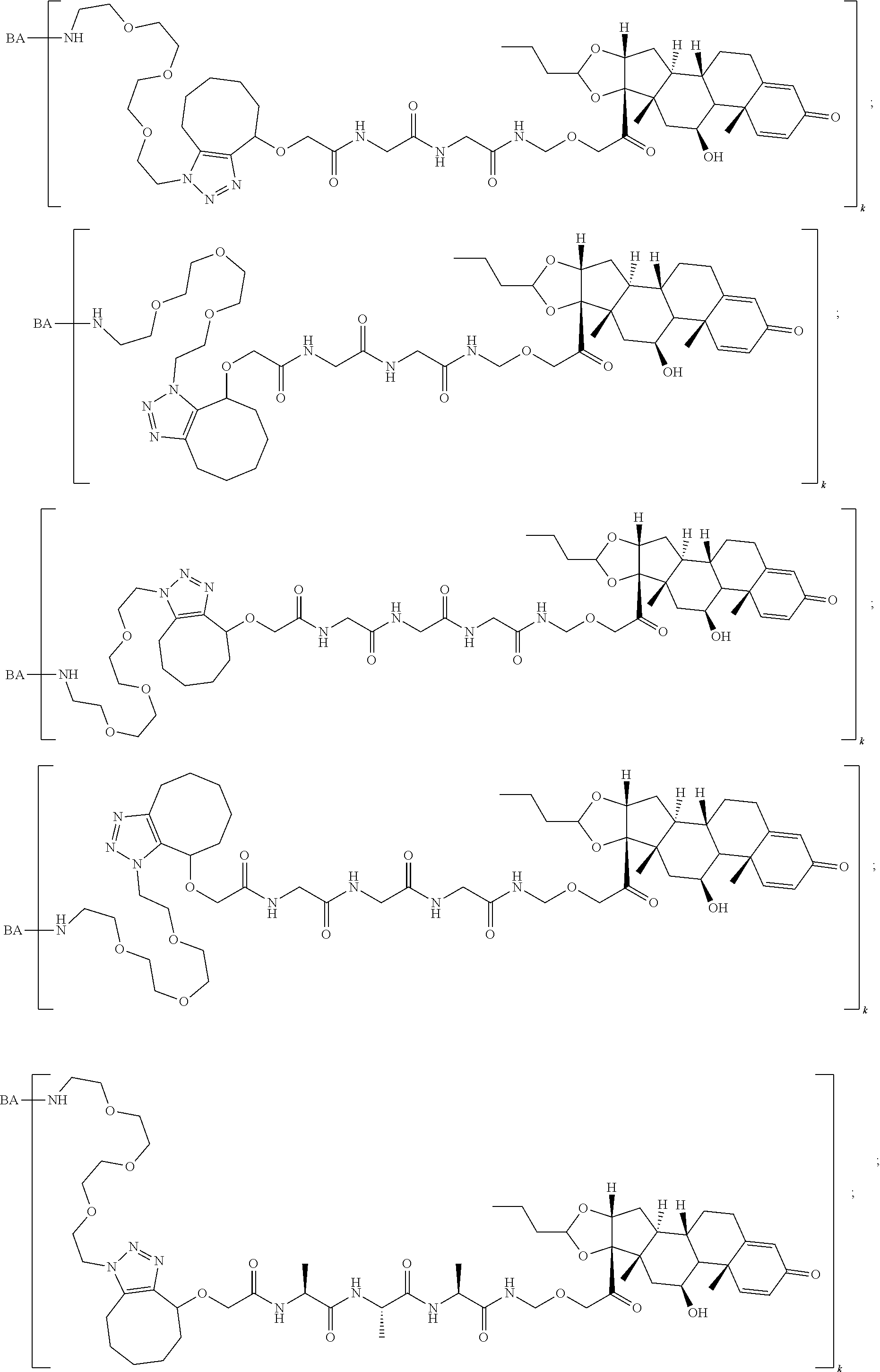

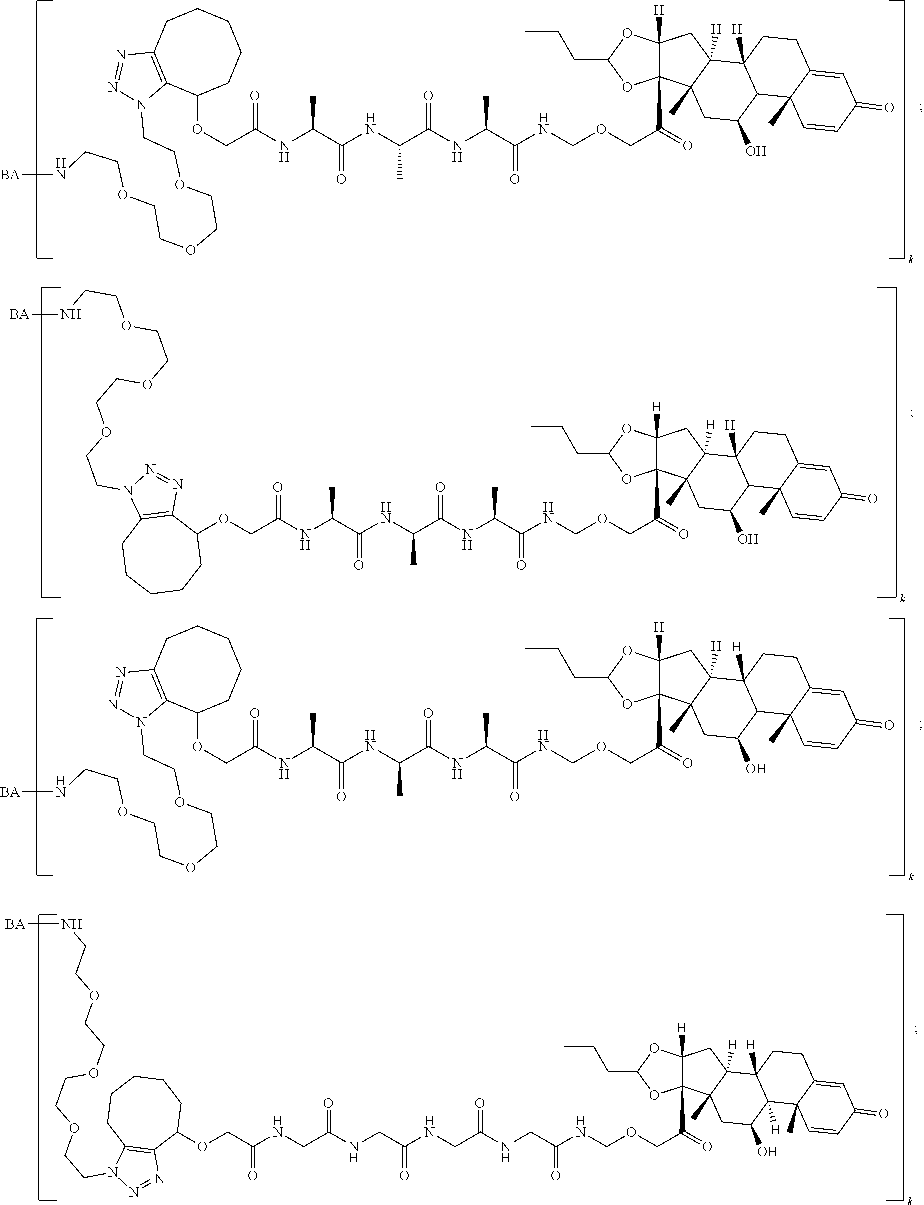

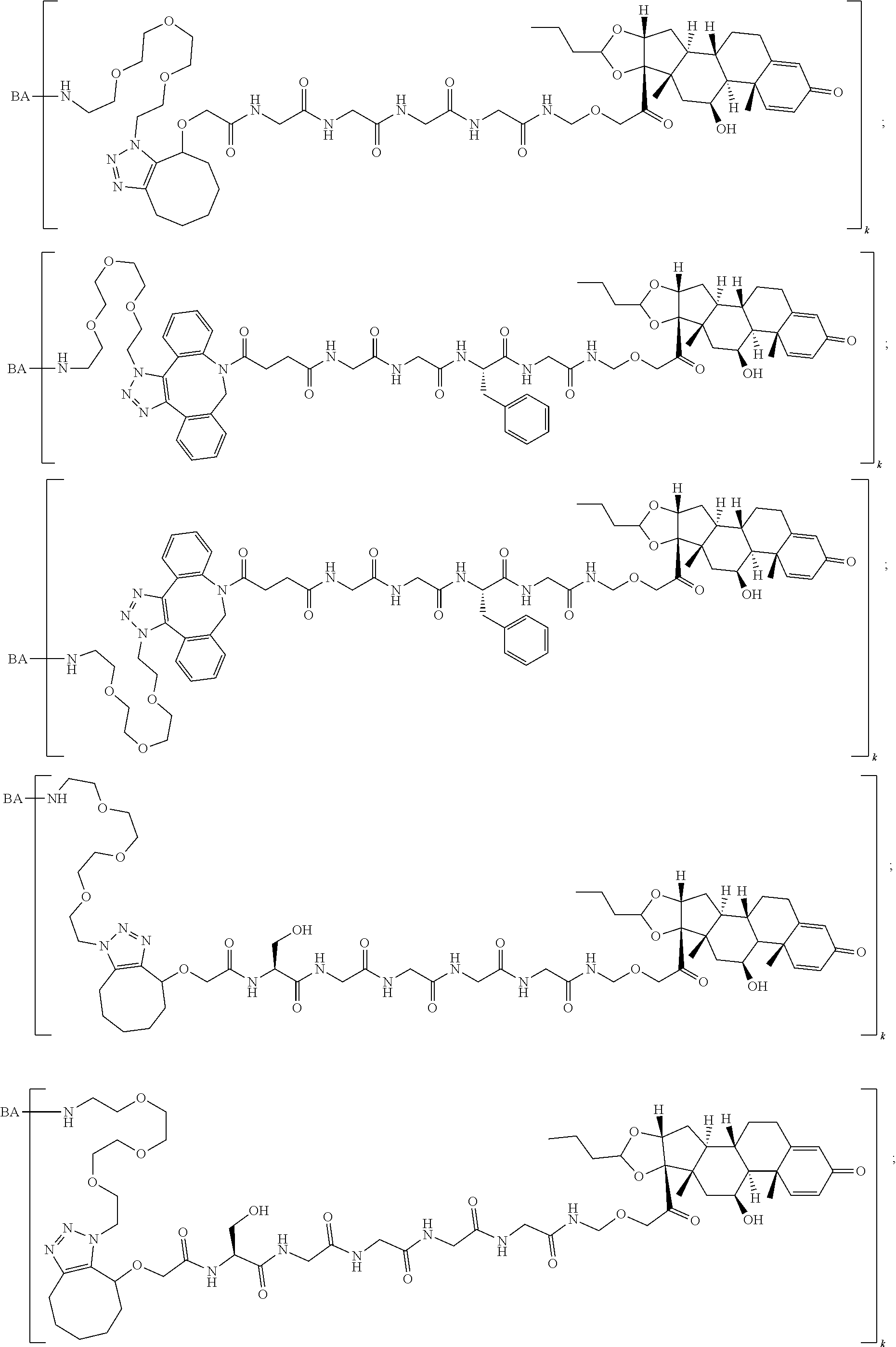

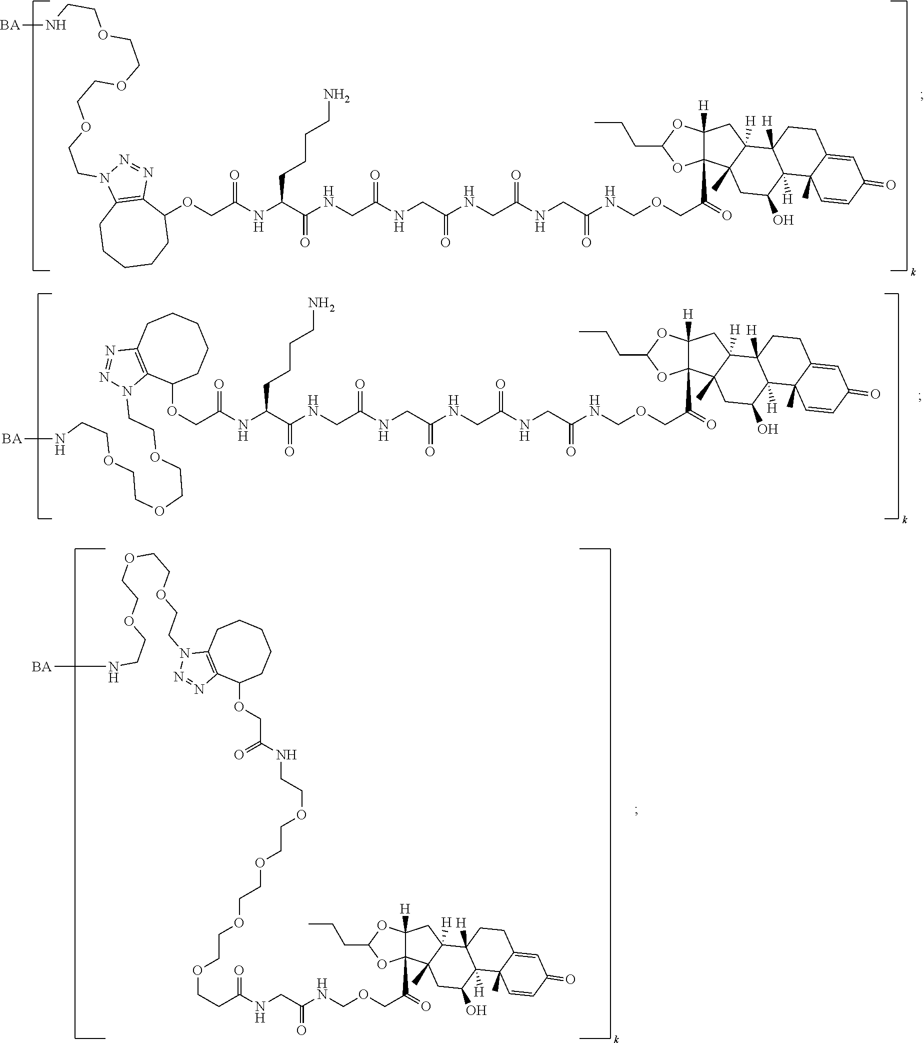

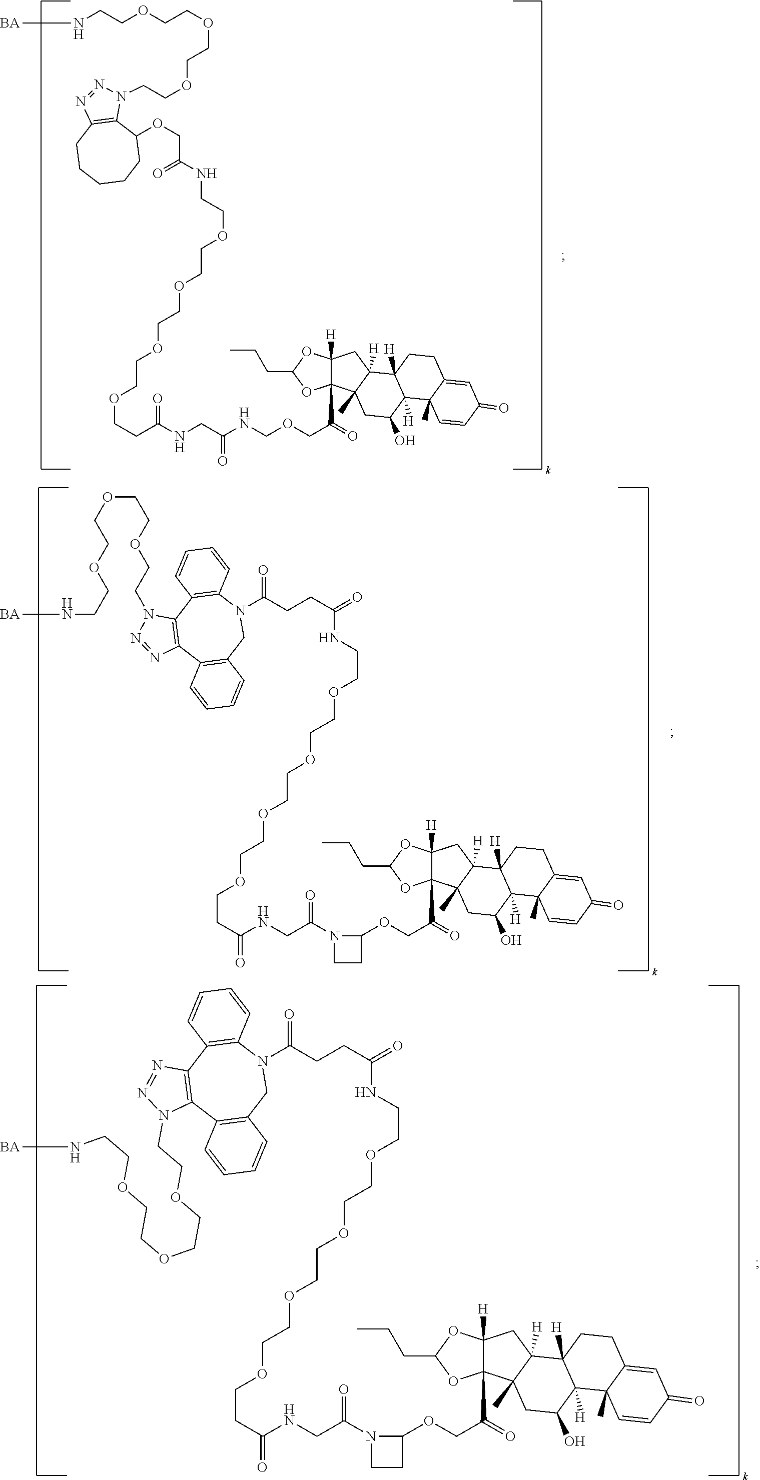

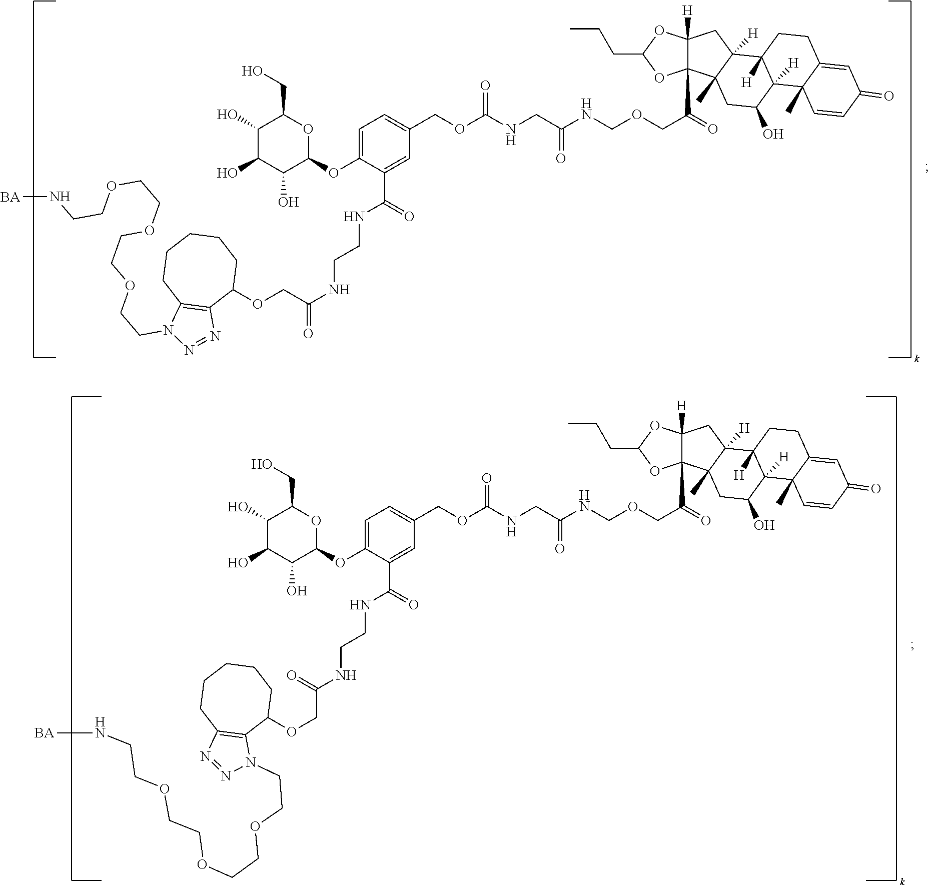

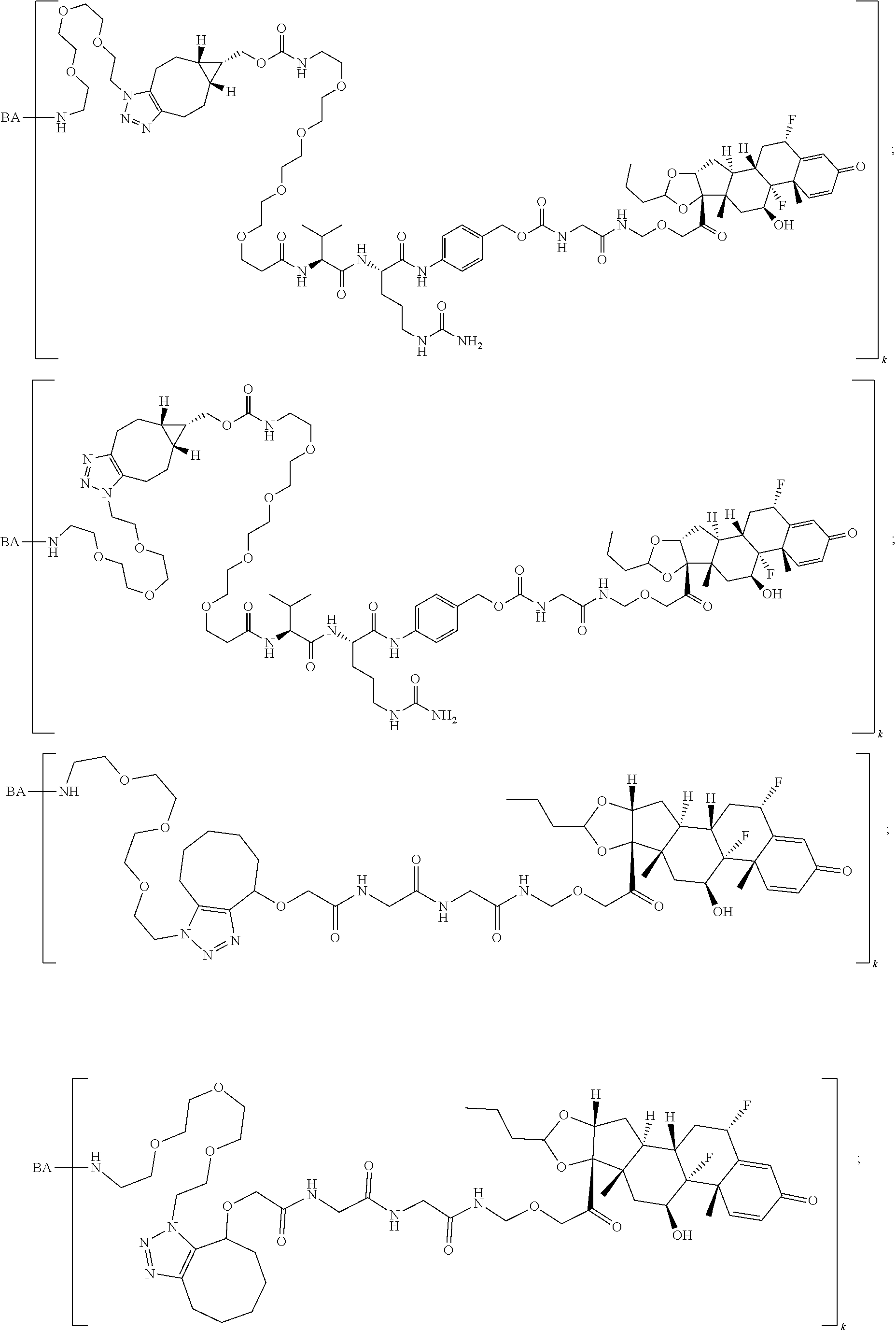

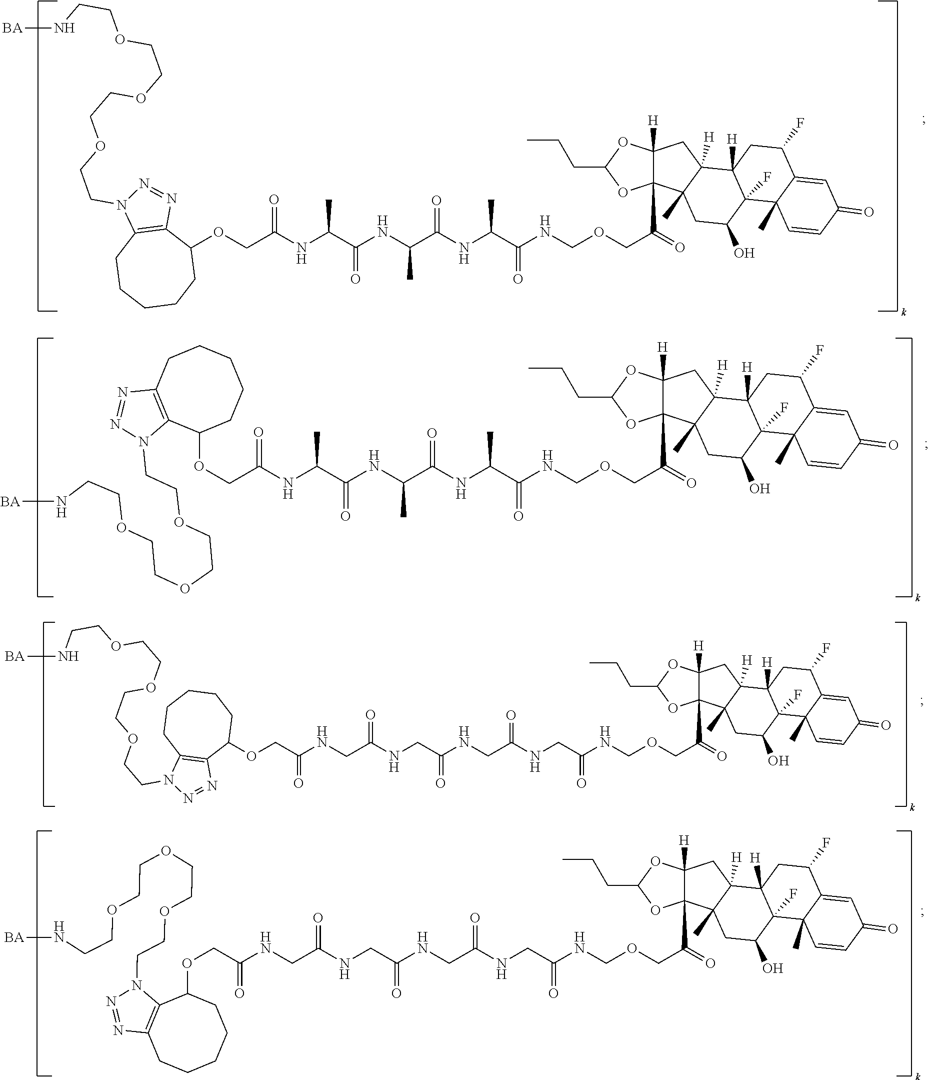

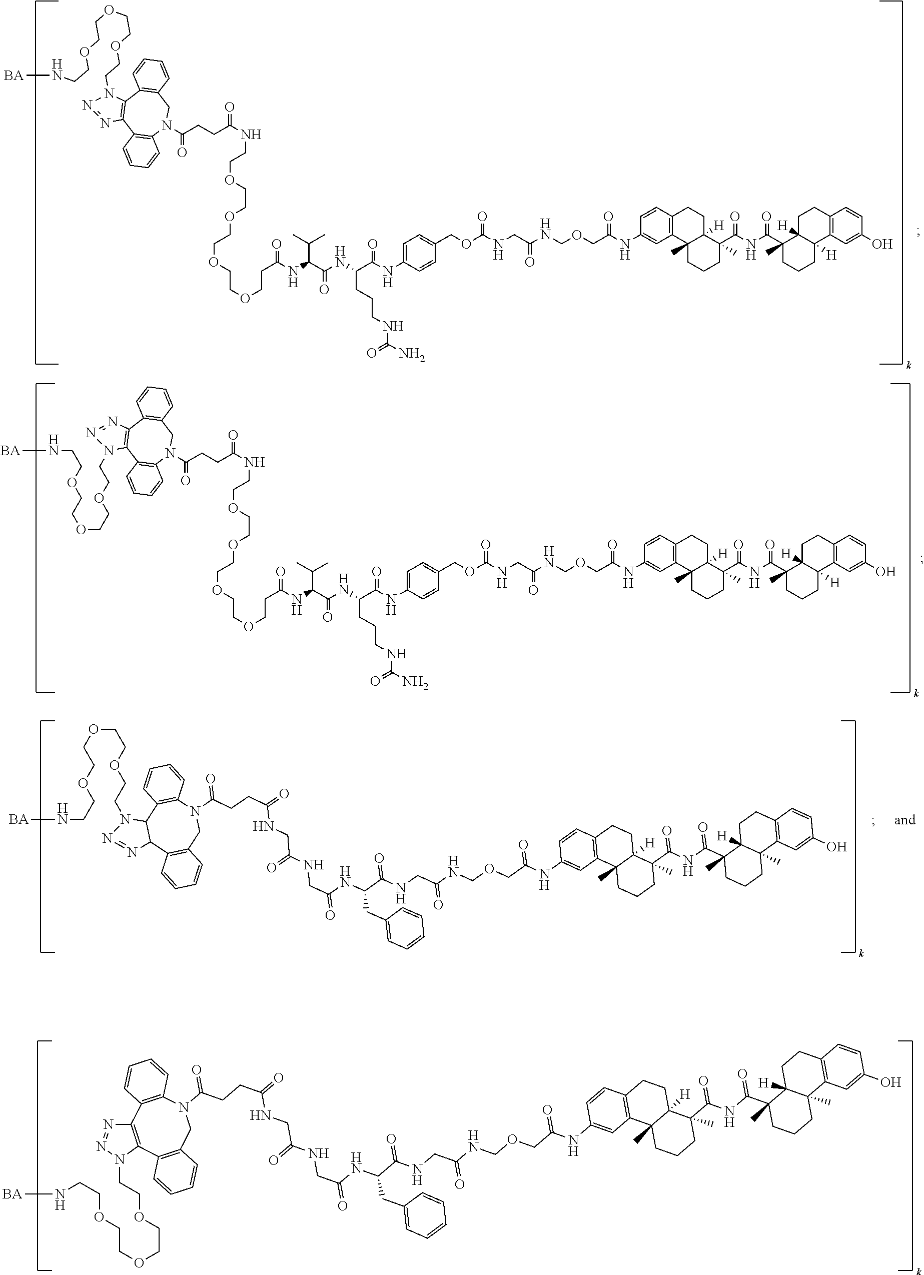

wherein k is 1, 2, 3, or 4. In certain embodiments within this paragraph, all diastereomers are contemplated. For example, in one embodiment, the stereochemistry at the hemiaminal ether (or hemiaminal, or N-acyl-N,O-acetal, wherein each name for this functional group is used interchangeably throughout this disclosure) is undefined or racemic. By way of further example, in one embodiment, the stereochemistry at the hemiaminal ether is (R)-. By way of further example, in one embodiment, the stereochemistry at the hemiaminal ether is (S)-. By way of further example, in one embodiment, the stereochemistry at the hemiaminal ether is (R)- in excess of (S)-. By way of further example, in one embodiment, the stereochemistry at the hemiaminal ether is (S)- in excess of (R)-. For example, in one embodiment, the stereochemistry at the acetal is undefined or racemic. By way of further example, in one embodiment, the stereochemistry at the acetal is (R)-. By way of further example, in one embodiment, the stereochemistry at the acetal is (S)-. By way of further example, in one embodiment, the stereochemistry at the acetal is (R)- in excess of (S)-. By way of further example, in one embodiment, the stereochemistry at the acetal is (S)- in excess of (R)-. In any of the embodiments in this paragraph, p is 1, 2, 3, 4, 5, 6, 7, 8, 9, or 10.

In certain embodiments, Formulae III, IIIa, IIIaa, IIIaaa, Tub, IIIbb, IIIbbb, and/or V is an antibody-drug conjugate including an antibody, or an antigen binding fragment thereof, where said antibody or antigen binding fragment thereof is conjugated to a compound of Formula I, Ia, Iaa, Iaaa, Ib, Ibb, Ibbb, II, IIa, IIaa, IIaaa, IIb, IIbb, and/or IIbbb. In another embodiment of Formulae III, IIIa, IIIaa, IIIaaa, IIIb, IIIbb, IIIbbb, and/or V, the antibody-drug conjugate is selected from the group consisting of

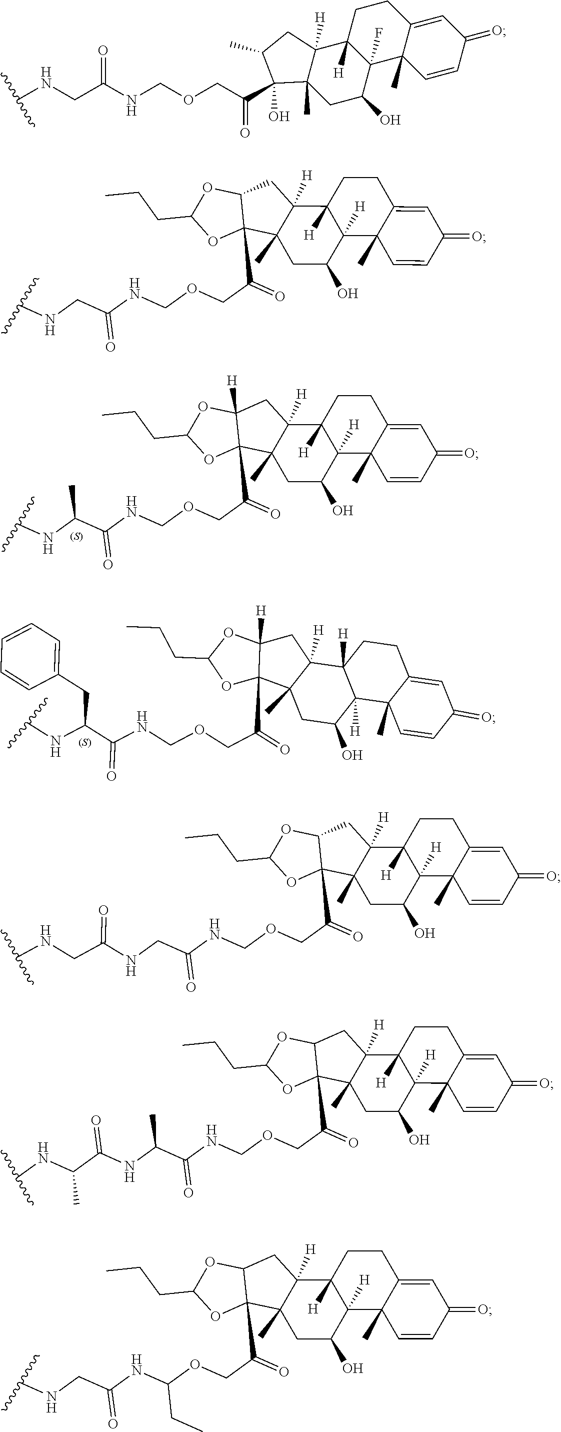

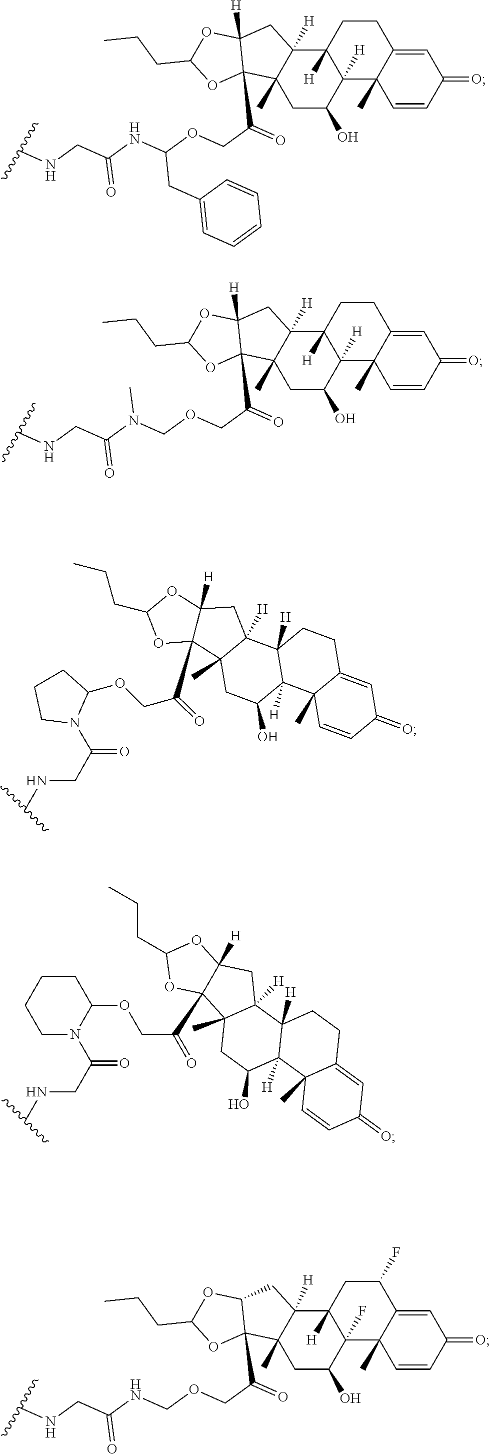

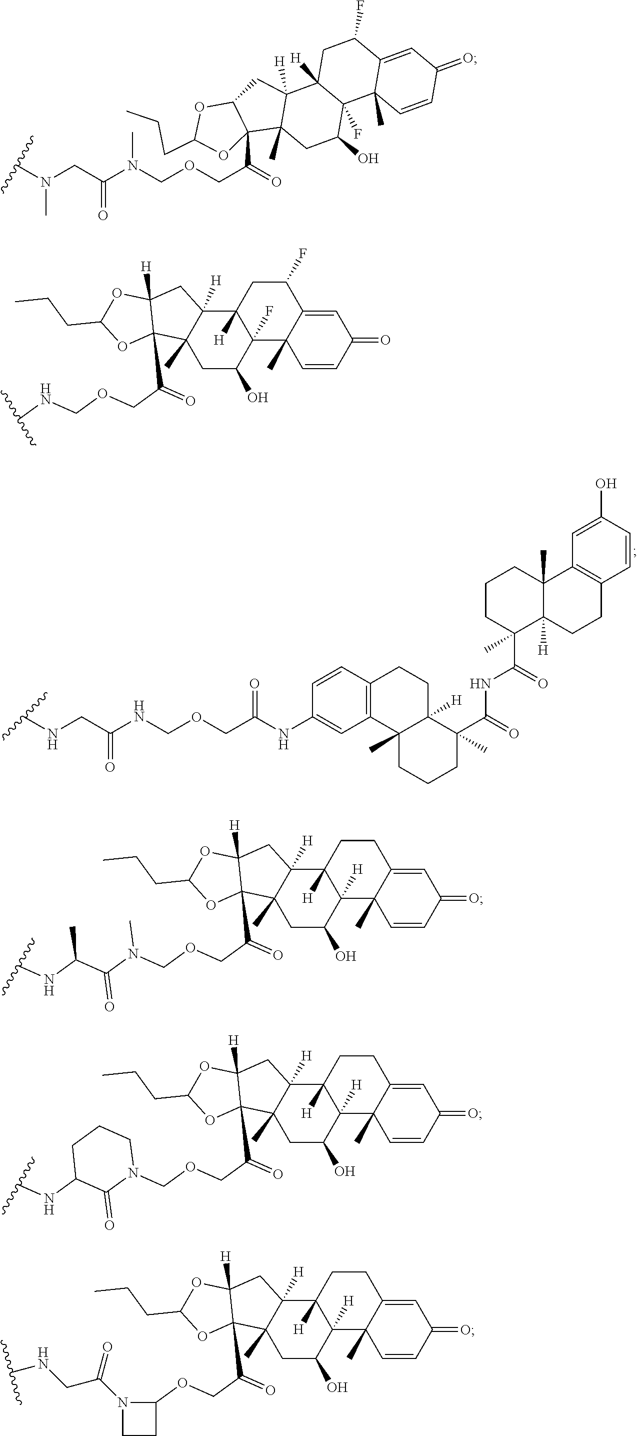

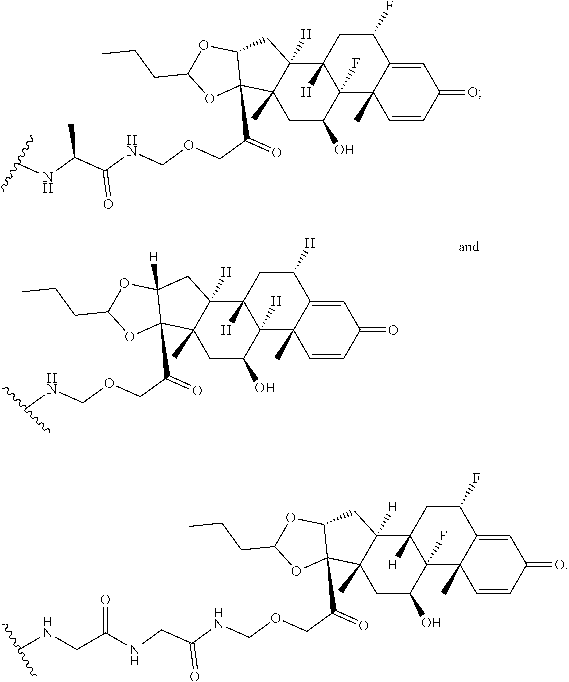

In certain embodiments within this paragraph, all diastereomers are contemplated. For example, in one embodiment, the stereochemistry at the hemiaminal ether (or hemiaminal, or N-acyl-N,O-acetal, wherein each name for this functional group is used interchangeably throughout this disclosure) is undefined or racemic. By way of further example, in one embodiment, the stereochemistry at the hemiaminal ether is (R)-. By way of further example, in one embodiment, the stereochemistry at the hemiaminal ether is (S)-. By way of further example, in one embodiment, the stereochemistry at the hemiaminal ether is (R)- in excess of (S)-. By way of further example, in one embodiment, the stereochemistry at the hemiaminal ether is (S)- in excess of (R)-. For example, in one embodiment, the stereochemistry at the acetal is undefined or racemic. By way of further example, in one embodiment, the stereochemistry at the acetal is (R)-. By way of further example, in one embodiment, the stereochemistry at the acetal is (S)-. By way of further example, in one embodiment, the stereochemistry at the acetal is (R)- in excess of (S)-. By way of further example, in one embodiment, the stereochemistry at the acetal is (S)- in excess of (R)-.

In one embodiment of Formula III, IIIa, IIIaa, IIIaaa, IIIb, IIIbb, or IIIbbb, BA is an antibody or antigen-binding fragment thereof. In another embodiment of Formula III, IIIa, IIIaa, IIIaaa, IIIb, IIIbb, or IIIbbb, BA is a transglutaminase-modified antibody or antigen-binding fragment thereof comprising at least one glutamine residue used for conjugation. In another embodiment of Formula III, IIIa, IIIaa, IIIaaa, IIIb, IIIbb, or IIIbbb, BA is a transglutaminase-modified antibody or antigen-binding fragment thereof comprising at least two glutamine residues used for conjugation. In another embodiment of Formula III, IIIa, IIIaa, IIIaaa, IIIb, IIIbb, or IIIbbb, BA is a transglutaminase-modified antibody or antigen-binding fragment thereof comprising at least four glutamine residues used for conjugation. In another embodiment of Formula III, IIIa, IIIaa, IIIaaa, IIIb, IIIbb, or IIIbbb, BA is a transglutaminase-modified antibody or antigen-binding fragment thereof wherein conjugation is at two Q295 residues; and k is 2. In another embodiment of Formula III, IIIa, IIIaa, IIIaaa, IIIb, IIIbb, or IIIbbb, BA is a transglutaminase-modified antibody or antigen-binding fragment thereof wherein conjugation is at two Q295 residues and two N297Q residues; and k is 4.

In certain embodiments, other antibody-drug conjugates are contemplated. Exemplary antibody-drug conjugates contemplated include, without limitation,

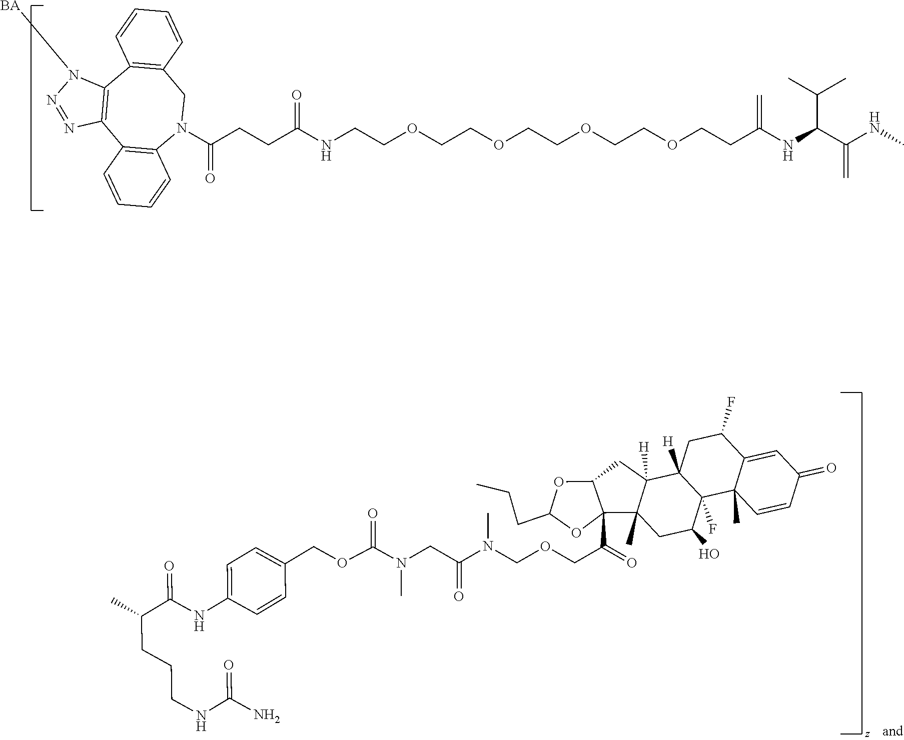

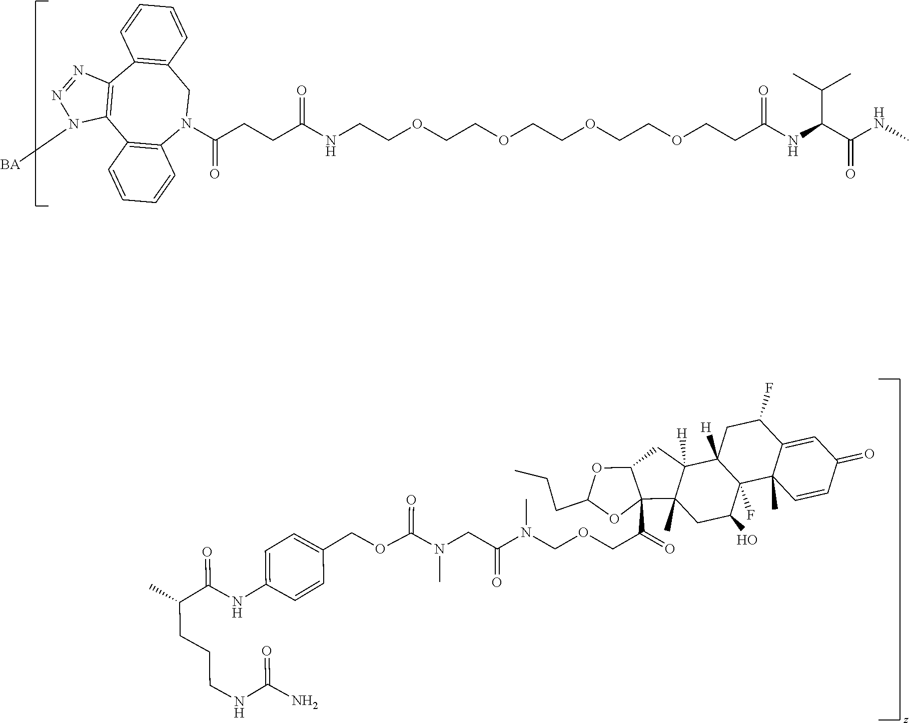

or a mixture thereof;

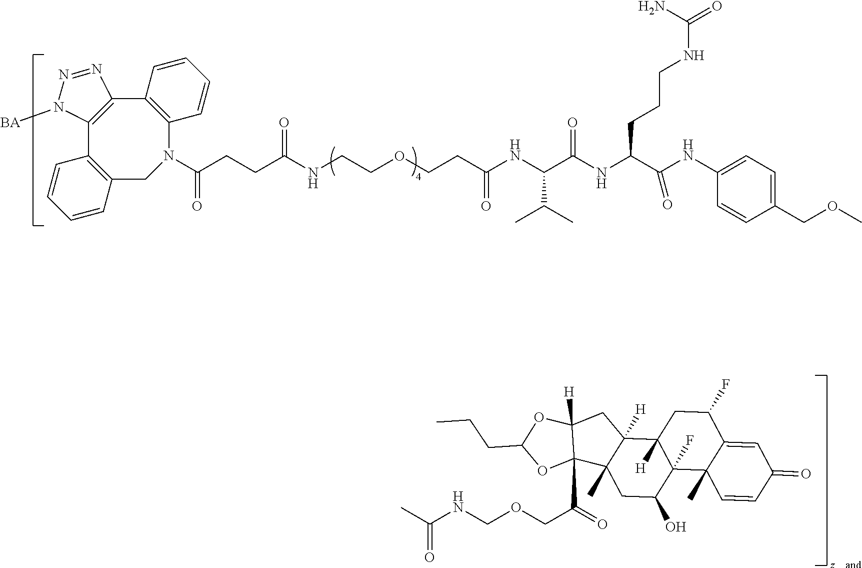

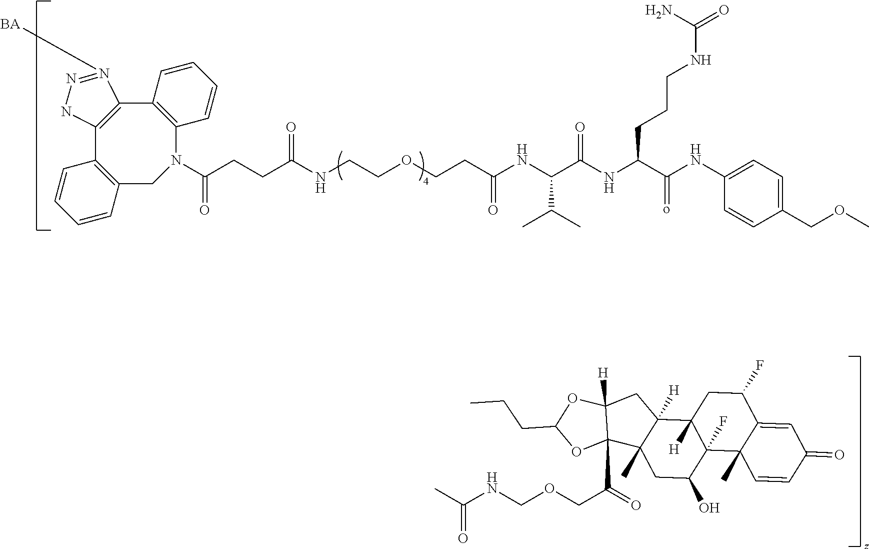

or a mixture thereof;

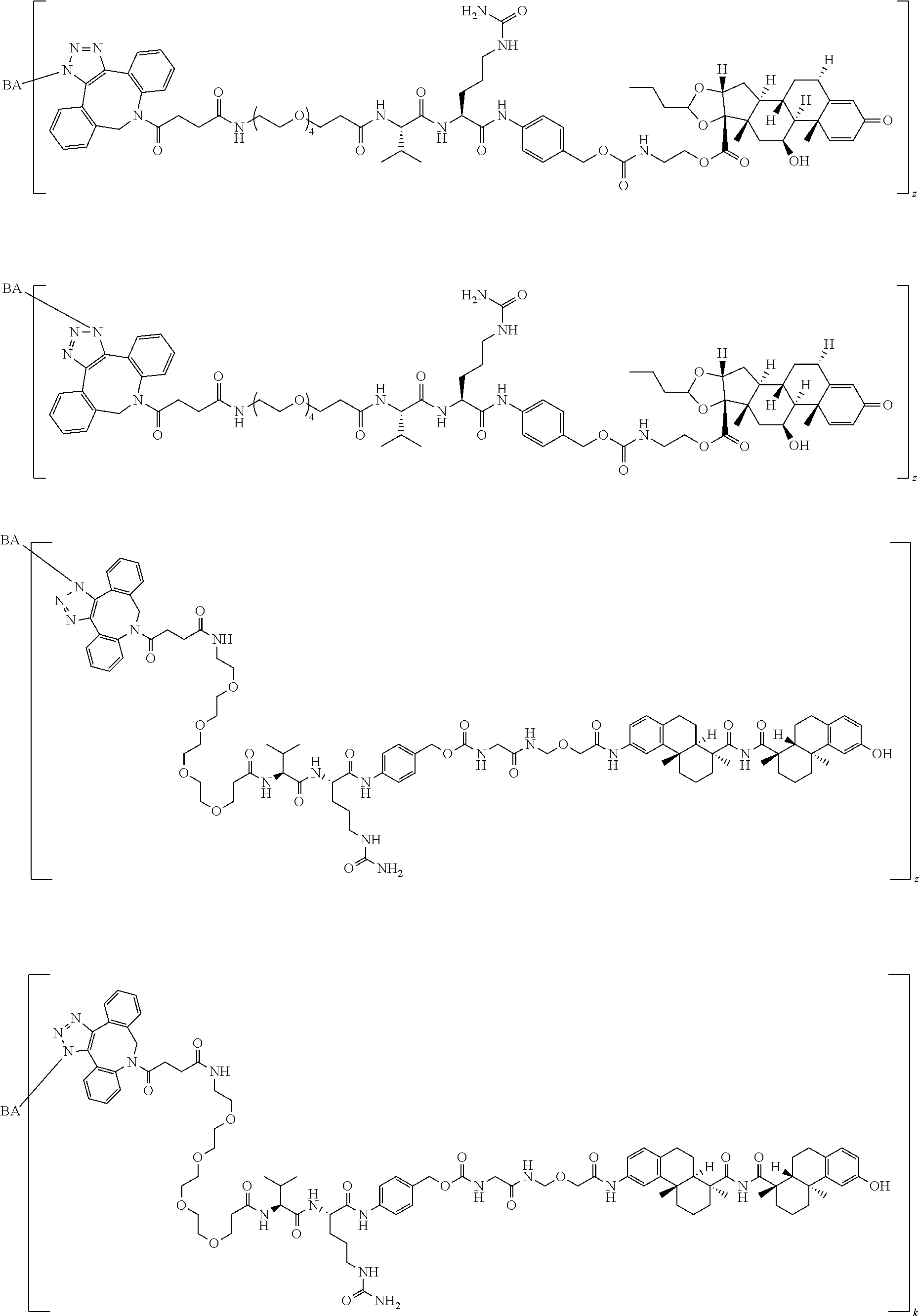

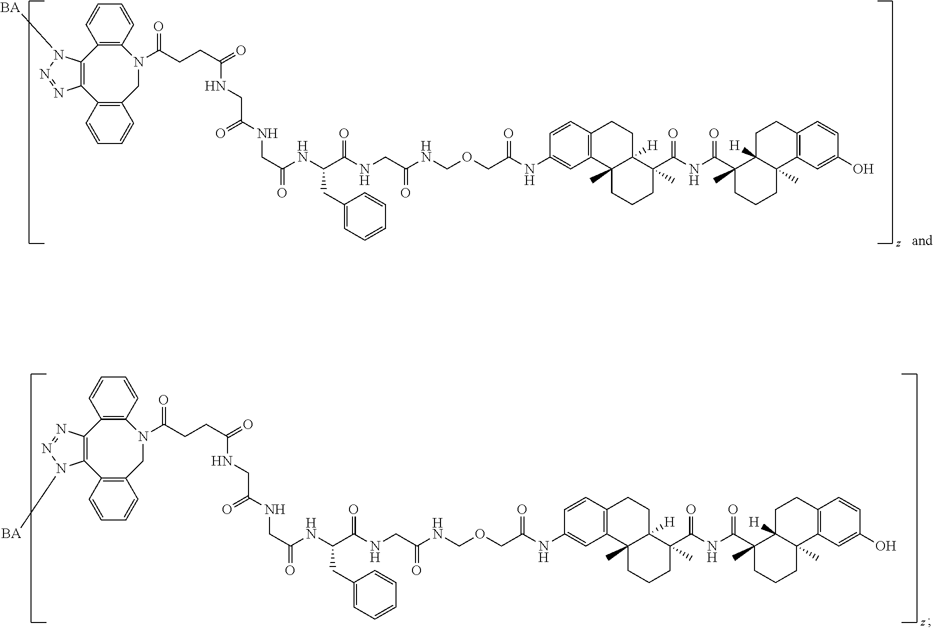

or a mixture thereof. In any of the embodiments in this paragraph, z or k is 1, 2, 3, or 4.

### Methods of Preparing Compounds, Prodrugs, or Payloads, and Linker Payloads

The compounds provided herein can be prepared, isolated, or obtained by any method apparent to those of skill in the art. Exemplary methods of preparation are described in detail in the Examples below. In certain embodiments, compounds provided herein can generally be prepared according to Schemes A-H, J, and K. In the following Exemplary Preparation Schemes, R^{1a}, R^{1b}, R², R³, R⁴, D* and n are described in the context of Formulae described herein.

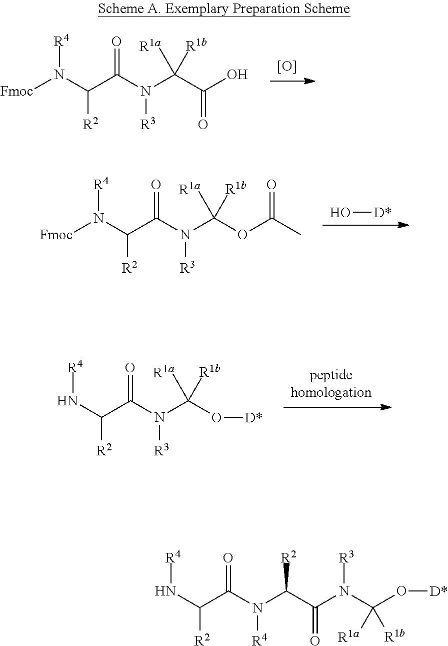

In Scheme A, amino acids were oxidatively decarboxylated, and then substituted with payloads (HO-D*). Payload derivatives where then subjected to further peptide homologations.

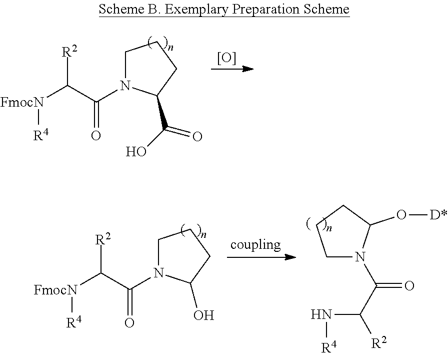

In Scheme B, amino acid derivatives were subjected to oxidative decarboxylation, followed by coupling with payloads (HO-D*).

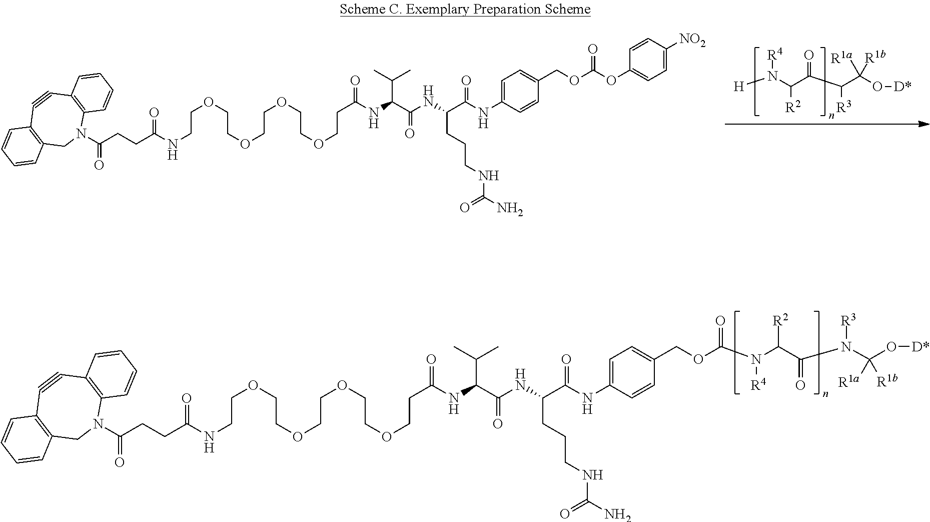

In Scheme C, linker-payloads were assembled using p-nitrophenyl carbonates.

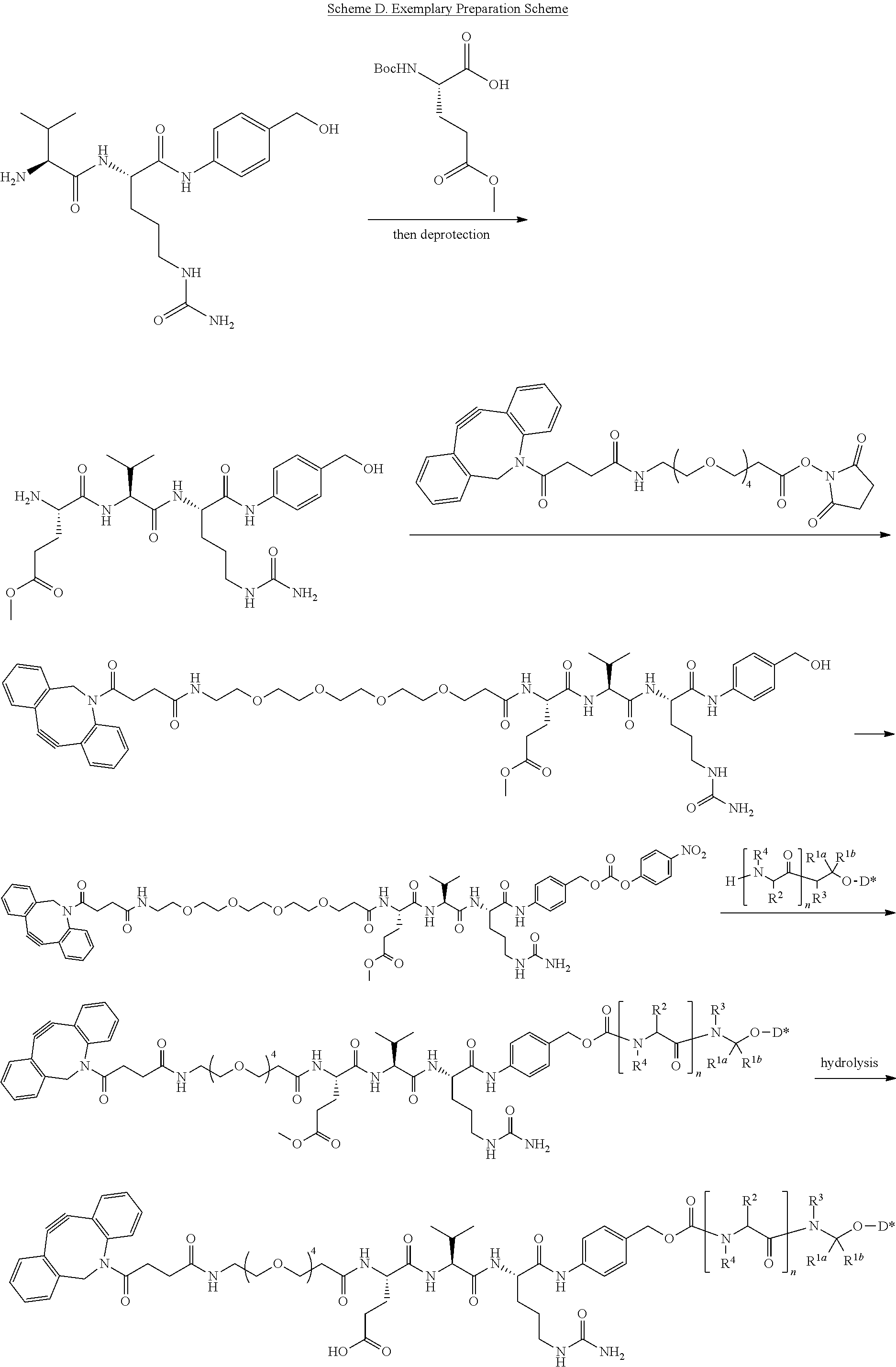

Scheme D also shows an alternative linker-payload synthesis. Dipeptide derivatives were homologated under peptide coupling conditions, followed by deprotection. Tripeptide coupling with N-hydroxysuccinimide esters provided the benzyl alcohol shown. The benzyl alcohol was converted to a p-nitrophenyl carbonate which was treated with a payload that provided the penultimate linker-payload. Final hydrolysis provided the linker-payload shown.

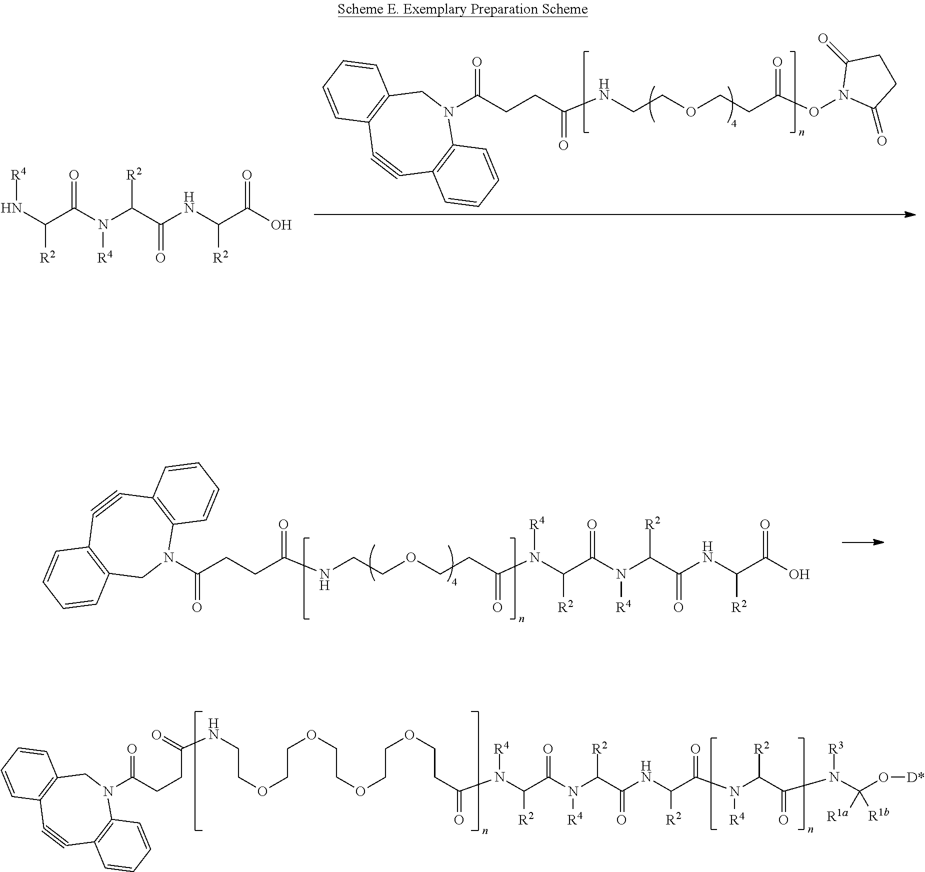

Scheme E shows yet another linker-payload synthesis. Tripeptide derivatives were coupled with N-hydroxysuccinimide esters and provided carboxylic acids like the one shown. The carboxylic acids were activated and coupled with payloads.

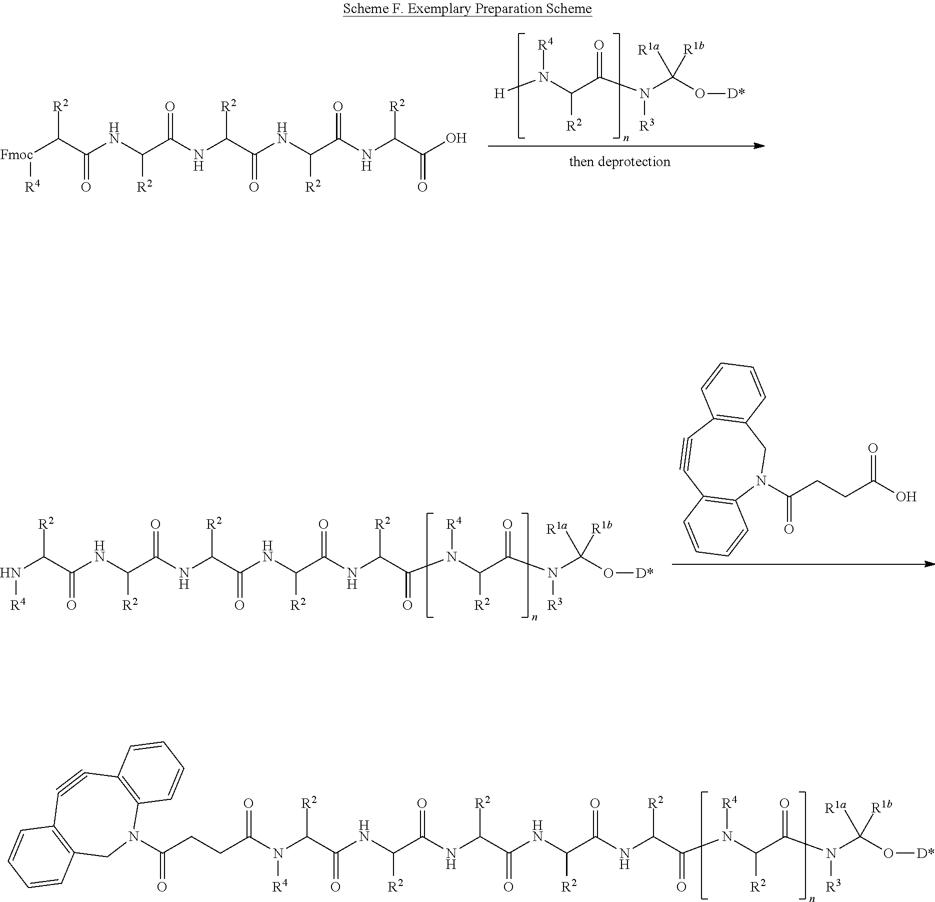

Other linker-payloads were synthesized according to Scheme F. Protected pentapeptides were coupled to payloads under peptide coupling conditions, and then deprotected. Subsequent peptide couplings with carboxylic acids provided the linker-payload shown.

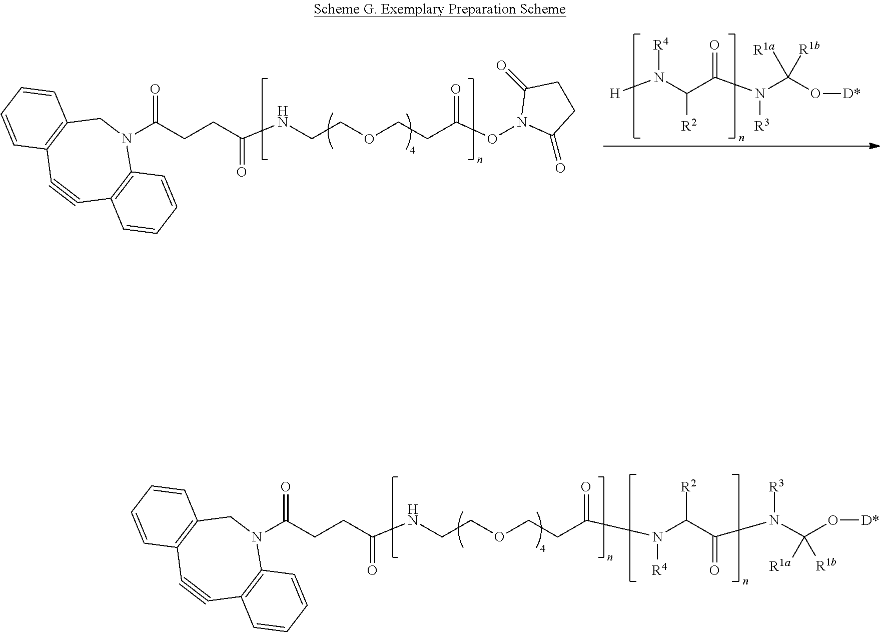

Yet other linker-payloads were synthesized according to Scheme G. Payloads were coupled to N-hydroxysuccinimide esters.

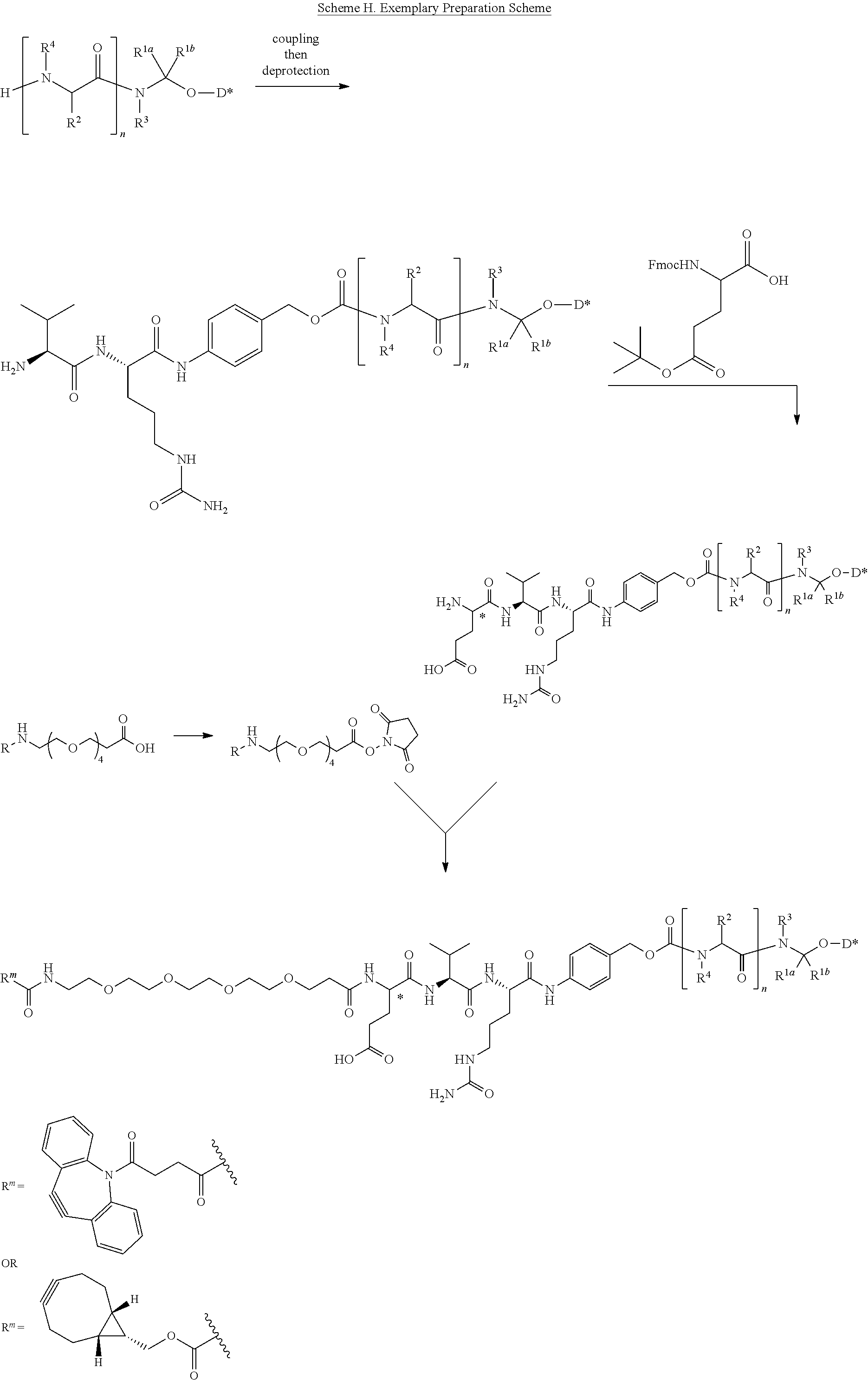

In Scheme H, the synthesis of multiple linker-payloads is shown. Payloads were coupled to protected p-nitrophenyl carbonates, followed by deprotection. Peptide homologation provided linker-payload intermediates terminating with (R)- or (S)- amino acids. These terminal amino acids were coupled to N-hydroxysuccinimide esters, derived from corresponding carboxylic acids, to provide the linker-payloads shown.

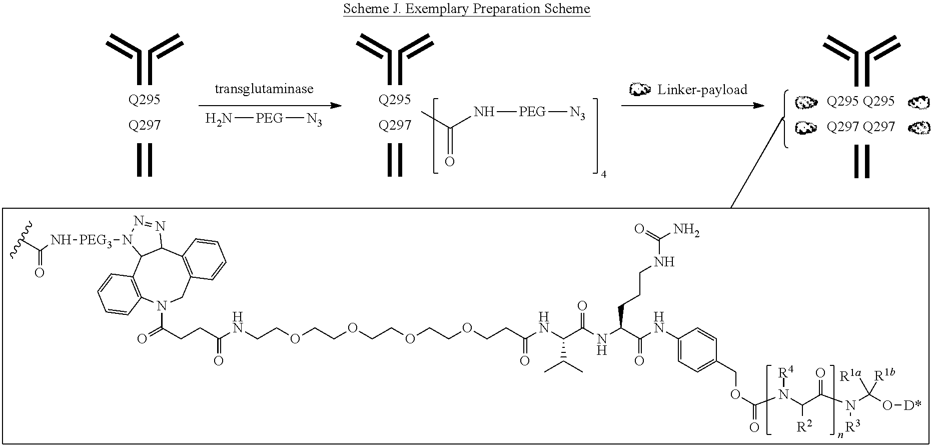

Scheme J shows general conjugation of linker-payloads to antibodies or antigen binding fragments thereof. Antibodies or antigen binding fragments thereof are modified via a transglutaminase to incorporate a terminal azide useful for participation in click chemistry with an alkyne. Accordingly, suitable linker-payloads are conjugated to the antibodies or antigen binding fragments thereof.

The conjugates described herein can be synthesized by coupling the linker-payloads described herein with a binding agent, for example, an antibody under standard conjugation conditions (see, e.g., Doronina et al. Nature Biotechnology 2003, 21, 778, which is incorporated herein by reference in its entirety). When the binding agent is an antibody, the antibody may be coupled to a linker-payload via one or more cysteine or lysine residues of the antibody. Linker-payloads can be coupled to cysteine residues, for example, by subjecting the antibody to a reducing agent, for example, dithiotheritol, to cleave the disulfide bonds of the antibody, purifying the reduced antibody, for example, by gel filtration, and subsequently treating the antibody with a linker-payload containing a suitable reactive moiety, for example, a maleimido group. Suitable solvents include, but are not limited to water, DMA, DMF, and DMSO. Linker-payloads containing a reactive group, for example, an activated ester or acid halide group, can be coupled to lysine residues of the antibody. Suitable solvents include, but are not limited to water, DMA, DMF, and DMSO. Conjugates can be purified using known protein techniques, including, for example, size exclusion chromatography, dialysis, and ultrafiltration/diafiltration.

Binding agents, for example antibodies, can also be conjugated via click chemistry reactions. In some embodiments of said click chemistry reactions, the linker-payload includes a reactive group, for example an alkyne, that is capable of undergoing a regioisomeric 1,3-cycloaddition reaction with an azide. Such suitable reactive groups are described above. The antibody includes one or more azide groups. Such antibodies include antibodies functionalized with, for example, azido-polyethylene glycol groups. In certain embodiments, such functionalized antibody is derived by treating an antibody having at least one glutamine residue, for example, heavy chain Gln295, with a primary amine compound in the presence of the enzyme transglutaminase. In certain embodiments, such functionalized antibody is derived by treating an antibody having at least one glutamine residue, for example, heavy chain Gln297, with a primary amine compound in the presence of the enzyme transglutaminase. Such antibodies include Asn297Gln (N297Q) mutants. In certain embodiments, such functionalized antibody is derived by treating an antibody having at least two glutamine residues, for example, heavy chain Gln295 and heavy chain Gln297, with a primary amine compound in the presence of the enzyme transglutaminase. Such antibodies include Asn297Gln (N297Q) mutants. In certain embodiments, the antibody has two heavy chains as described in this paragraph for a total of two or a total of four glutamine residues.

In certain embodiments, the antibody comprises two glutamine residues, one in each heavy chain. In particular embodiments, the antibody comprises a Q295 residue in each heavy chain. In further embodiments, the antibody comprises one, two, three, four, five, six, seven, eight, or more glutamine residues. These glutamine residues can be in heavy chains, light chains, or in both heavy chains and light chains. These glutamine residues can be wild-type residues, or engineered residues. The antibodies can be prepared according to standard techniques.

Those of skill will recognize that antibodies are often glycosylated at residue N297, near residue Q295 in a heavy chain sequence. Glycosylation at residue N297 can interfere with a transglutaminase at residue Q295 (Dennler et al., supra). Accordingly, in advantageous embodiments, the antibody is not glycosylated. In certain embodiments, the antibody is deglycoslated or aglycosylated. In particular embodiments, an antibody heavy chain has an N297 mutation. Alternatively stated, the antibody is mutated to no longer have an asparagine residue at position 297. In particular embodiments, an antibody heavy chain has an N297Q mutation. Such an antibody can be prepared by site-directed mutagenesis to remove or disable a glycosylation sequence or by site-directed mutagenesis to insert a glutamine residue at a site apart from any interfering glycosylation site or any other interfering structure. Such an antibody also can be isolated from natural or artificial sources.

The antibody without interfering glycosylation is then reacted or treated with a primary amine compound. In certain embodiments, an aglycosylated antibody is reacted or treated with a primary amine compound to produce a glutaminyl-modified antibody. In certain embodiments, a deglycosylated antibody is reacted or treated with a primary amine compound to produce a glutaminyl-modified antibody.

The primary amine can be any primary amine that is capable of forming a covalent bond with a glutamine residue in the presence of a transglutaminase. Useful primary amines are described herein. The transglutaminase can be any transglutaminase deemed suitable by those of skill in the art. In certain embodiments, the transglutaminase is an enzyme that catalyzes the formation of an isopeptide bond between a free amine group on the primary amine compound and the acyl group on the side chain of a glutamine residue. Transglutaminase is also known as protein-glutamine-γ-glutamyltransferase. In particular embodiments, the transglutaminase is classified as EC 2.3.2.13. The transglutaminase can be from any source deemed suitable. In certain embodiments, the transglutaminase is microbial. Useful transglutaminases have been isolated from Streptomyces mobaraense, Streptomyces cinnamoneum, Streptomyces griseo-carneum, Streptomyces lavendulae, and Bacillus subtilis. Non-microbial transglutaminases, including mammalian transglutaminases, can also be used. In certain embodiments, the transglutaminase can be produced by any technique or obtained from any source deemed suitable by the practitioner of skill. In particular embodiments, the transglutaminase is obtained from a commercial source.

In particular embodiments, the primary amine compound comprises a reactive group capable of further reaction after transglutamination. In these embodiments, the glutaminyl-modified antibody can be reacted or treated with a reactive payload compound or a reactive linker-payload compound to form an antibody-payload conjugate. In certain embodiments, the primary amine compound comprises an azide.

In certain embodiments, the glutaminyl-modified antibody is reacted or treated with a reactive linker-payload to form an antibody-payload conjugate. The reaction can proceed under conditions deemed suitable by those of skill in the art. In certain embodiments, the glutaminyl-modified antibody is contacted with the reactive linker-payload compound under conditions suitable for forming a bond between the glutaminyl-modified antibody and the linker-payload compound. Suitable reaction conditions are well known to those in the art. Exemplary reactions are provided in the Examples below. Accordingly, provided herein is a method of preparing an antibody-drug conjugate comprising contacting a binding agent, as described herein, with a linker-payload, also as described herein.

### Pharmaceutical Compositions and Methods of Treatment

Provided herein are methods of treating and preventing diseases, conditions, or disorders comprising administering a therapeutically or prophylactically effective amount or one or more of the compounds disclosed herein, for example, one or more of the compounds of a formula provided herein. Diseases, disorders, and/or conditions include, but are not limited to, those associated with the antigens listed herein.

The compounds described herein can be administered alone or together with one or more additional therapeutic agents. The one or more additional therapeutic agents can be administered just prior to, concurrent with, or shortly after the administration of the compounds described herein. The present disclosure also includes pharmaceutical compositions comprising any of the compounds described herein in combination with one or more additional therapeutic agents, and methods of treatment comprising administering such combinations to subjects in need thereof.

Suitable additional therapeutic agents include, but are not limited to: a second glucocorticoid, steroid, LXR modulator, an inflammatory therapeutic agent, an autoimmune therapeutic agent, a hormone, a biologic, or a monoclonal antibody. Suitable therapeutic agents also include, but are not limited to any pharmaceutically acceptable salts, acids, or derivatives of a compound set forth herein. The compounds described herein can also be administered and/or co-formulated in combination with antivirals, antibiotics, analgesics, corticosteroids, steroids, oxygen, antioxidants, COX inhibitors, cardioprotectants, metal chelators, IFN-gamma, and/or NSAIDs.

In some embodiments of the methods described herein, multiple doses of a compound described herein (or a pharmaceutical composition comprising a combination of a compound described herein and any of the additional therapeutic agents mentioned herein) may be administered to a subject over a defined time course. The methods according to this embodiment of the disclosure comprise sequentially administering to a subject multiple doses of a compound described herein. As used herein, “sequentially administering” means that each dose of the compound is administered to the subject at a different point in time, e.g., on different days separated by a predetermined interval (e.g., hours, days, weeks, or months). The present disclosure includes methods which comprise sequentially administering to the patient a single initial dose of a compound described herein, followed by one or more secondary doses of the compound, and optionally followed by one or more tertiary doses of the compound.

The terms “initial dose,” “secondary doses,” and “tertiary doses,” refer to the temporal sequence of administration of the compounds described herein. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses can all include the same amount the compound described herein, but generally can differ from one another in terms of frequency of administration. In certain embodiments, the amount of the compound included in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose the compound which is administered to a patient prior to the administration of the very next dose in the sequence with no intervening doses.

The methods according to this embodiment of the disclosure may comprise administering to a patient any number of secondary and/or tertiary doses of the compound. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient. The administration regimen may be carried out indefinitely over the lifetime of a particular subject, or until such treatment is no longer therapeutically needed or advantageous.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the disclosure, the frequency at which the secondary and/or tertiary doses are administered to a patient can vary over the course of the treatment regimen. The frequency of administration may also be adjusted during the course of treatment by a physician depending on the needs of the individual patient following clinical examination.

The present disclosure includes administration regimens in which 2 to 6 loading doses are administered to a patient at a first frequency (e.g., once a week, once every two weeks, once every three weeks, once a month, once every two months, etc.), followed by administration of two or more maintenance doses to the patient on a less frequent basis. For example, according to this embodiment of the disclosure, if the loading doses are administered at a frequency of once a month, then the maintenance doses may be administered to the patient once every six weeks, once every two months, once every three months, etc.

The present disclosure includes pharmaceutical compositions of the compounds and/or conjugates described herein, e.g., the compounds Formulae I, Ia, Iaa, Iaaa, Ib, Ibb, Ibbb, II, IIa, IIaa, IIaaa, IIb, IIbb, IIbbb, III, IIIa, IIIaa, IIIaaa, IIIb, IIIbb, IIIbbb, IV, and/or V, e.g., compositions comprising a compound described herein, a salt, stereoisomer, regioisomer, polymorph thereof, and a pharmaceutically acceptable carrier, diluent, and/or excipient. Examples of suitable carriers, diluents and excipients include, but are not limited to, buffers for maintenance of proper composition pH (e.g., citrate buffers, succinate buffers, acetate buffers, phosphate buffers, lactate buffers, oxalate buffers, and the like), carrier proteins (e.g., human serum albumin), saline, polyols (e.g., trehalose, sucrose, xylitol, sorbitol, and the like), surfactants (e.g., polysorbate 20, polysorbate 80, polyoxolate, and the like), antimicrobials, and antioxidants. In one embodiment, provided is a pharmaceutical composition including the compounds of any of Formulae I, Ia, Iaa, Iaaa, Ib, Ibb, Ibbb, II, IIa, IIaa, IIaaa, IIb, IIbb, IIbbb, III, IIIa, IIIaa, IIIaaa, IIIb, IIIbb, IIIbbb, IV, and/or V, and a pharmaceutically acceptable excipient, carrier, or diluent.

In some examples, set forth herein is a method of treating a disease, disorder or condition including administering to a patient having said disorder a therapeutically effective amount of a compound set forth herein, or a pharmaceutical composition thereof.

In some examples, set forth herein is a method of treating a disease, disorder or condition including administering to a patient having said disorder a therapeutically effective amount of a compound of Formulae I, Ia, Iaa, Iaaa, Ib, Ibb, Ibbb, II, IIa, IIaa, IIaaa, IIb, IIbb, IIbbb, III, IIIa, IIIaa, IIIaaa, IIIb, IIIbb, IIIbbb, IV, and/or V, or a pharmaceutical composition thereof.

In some examples, set forth herein are methods of treating a disease, disorder, or condition associated with the glucocorticoid receptor comprising administering a compound of Formulae I, Ia, Iaa, Iaaa, Ib, Ibb, Ibbb, II, IIa, IIaa, IIaaa, IIb, IIbb, IIbbb, III, IIIa, IIIaa, IIIaaa, IIIb, IIIbb, IIIbbb, IV, and/or V, to a patient having said disease, disorder, or condition, and combinations thereof.

The present disclosure includes methods of preventing certain disorders or conditions comprising administering a therapeutically effective amount of one or more of the compounds disclosed herein (i.e., prophylactic uses). Examples include, but are not limited to preventing cytokine release syndrome for CD3 bispecifics, and adoptive cellular therapies such as CAR T cells, systemic IL-2 administration, graft-versus-host disease, and post-operative nausea and vomiting. Examples also include, but are not limited to, therapeutic antibodies such as alemtuzumab, muromonab, rituximab, tosituzumab, and agonistic antibodies where immune stimulation might be part of the intended mechanism of action.

In some embodiments, the disease, disorder, or condition is an allergic state, including, but not limited to, asthma, atopic dermatitis, contact dermatitis, drug hypersensitivity reactions, anaphylactic rhinitis, perennial or seasonal allergic rhinitis, and serum sickness; dermatologic diseases and conditions including, but not limited to, skin itching, seborrheic dermatitis, neurodermatitis, bullous dermatitis herpetiformis, exfoliative erythroderma, mycosis fungoides, pemphigus, and severe erythema multiforme (Stevens-Johnson syndrome); endocrine disorders including, but not limited to, primary or secondary adrenocortical insufficiency, congenital adrenal hyperplasia, hypercalcemia associated with cancer, and nonsuppurative thyroiditis; gastrointestinal diseases; hematologic disorders including, but not limited to, acquired (autoimmune) hemolytic anemia, congenital (erythroid) hypoplastic anemia (Diamond-Blackfan anemia), idiopathic thrombocytopenic purpura in adults, pure red cell aplasia, and secondary thrombocytopenia; trichinosis; tuberculous meningitis with subarachnoid block or impending block; neoplastic diseases including, but not limited to, leukemias and lymphomas; nervous system disorders including, but not limited to, acute exacerbations of multiple sclerosis, cerebral edema associated with primary or metastatic brain tumor, craniotomy, or head injury; ophthalmic diseases including, but not limited to, sympathetic ophthalmia, temporal arteritis, uveitis, and ocular inflammatory conditions unresponsive to topical corticosteroids; renal diseases including, but not limited to, for inducing a diuresis or remission of proteinuria in idiopathic nephrotic syndrome or that due to lupus erythematosus; respiratory diseases including, but not limited to, berylliosis, fulminating or disseminated pulmonary tuberculosis when used concurrently with appropriate antituberculous chemotherapy, idiopathic eosinophilic pneumonias, symptomatic sarcoidosis; and Rheumatic disorders including, but not limited to, use as adjunctive therapy for short-term administration (to tide the patient over an acute episode or exacerbation) in acute gouty arthritis, acute rheumatic carditis, ankylosing spondylitis, psoriaticarthritis, rheumatoid arthritis, including juvenile rheumatoid arthritis, and for use in dermatomyositis, polymyositis, and systemic lupus erythematosus.

In some examples, set forth herein is a method for treating a disease, disorder, or condition selected from an autoimmune disease, an allergy, arthritis, asthma, a breathing disorder, a blood disorder, a cancer, a collagen disease, a connective tissue disorder, a dermatological disease, an eye disease, an endocrine problem, an immunological disease, an inflammatory disease, an intestinal disorder, a gastrointestinal disease, a neurological disorder, an organ transplant condition, a rheumatoid disorder, a skin disorder, a swelling condition, a wound healing condition, and combinations thereof, comprising administering a steroid payload or conjugate thereof described herein.

In some examples, the autoimmune disorder is selected from multiple sclerosis, autoimmune hepatitis, shingles, systemic lupus erythematosus (i.e., lupus), myasthenia gravis, Duchenne muscular dystrophy, and sarcoidosis. In some examples, the breathing disorder is selected from asthma, chronic respiratory disease, chronic obstructive pulmonary disease, bronchial inflammation, and acute bronchitis. In some examples, the cancer is selected from leukemia, lymphoblastic leukemia, acute lymphoblastic leukemia, chronic lymphoblastic leukemia, Hodgkin's lymphoma, Non-Hodgkin's lymphoma (NHL), and multiple myeloma. In some examples, the collagen disease is systemic lupus erythematosus. In some examples, the eye disease is keratitis. In some examples, the endocrine problem is selected from Addison's Disease, adrenal insufficiency, adrenal cortical dysfunction, adrenocortical, and congenital adrenal hyperplasia. In some examples, the inflammatory disease is selected from inflammation after cataract surgery, joint inflammation, immune inflammation, tendon inflammation, bursitis, epicondylitis, Crohn's disease, inflammatory bowels disease, lipid pneumonitis thyroiditis, urticaria (hives), pericarditis, nephrotic syndrome, and uveitis. In some examples, the intestinal disorder is selected from ulcerative colitis, Crohn's disease, and inflammatory bowel disease. In some examples, the rheumatoid disorder is selected from rheumatoid arthritis, polymyalgia rheumatic, psoriatic arthritis, ankylosing spondylitis, and systemic lupus erythematosus. In some examples, the skin disorder is selected from psoriasis, eczema, and poison ivy. In some examples, the neurological disorder is chronic inflammatory demyelinating polyradiculoneuropathy.

In some embodiments, the compounds described herein are administered to a patient to treat an acute inflammatory event including, but not limited to, shock, brain edema, and graft-vs-host disease. In some embodiments, the compounds described herein are administered to treat lympholytic effects including, but not limited to, those associated with hematological malignancies, for example, leukemias, lymphomas, and myelomas.

In some examples, set forth herein is a method for reducing inflammation in a subject in need thereof, comprising administering to a subject in need thereof a therapeutically effective amount of a steroid or conjugate thereof described herein. In some examples, set forth herein is a method for modulating the immune system in a subject in need thereof, comprising administering to a subject in need thereof a therapeutically effective amount of a steroid or conjugate thereof described herein. In some examples, set forth herein is a method for modulating cortisol levels in a subject in need thereof, comprising administering to a subject in need thereof a therapeutically effective amount of a steroid or conjugate thereof described herein. In some examples, set forth herein is a method of reducing lymphocyte migration in a subject in need thereof, comprising administering to a subject in need thereof a therapeutically effective amount of a steroid or conjugate thereof described herein. In some examples, set forth herein is a method of treating hypercalcemia due to cancer, Meniere's disease, a migraine headache, a cluster headache, a severe aphthous ulcer, laryngitis, severe tuberculosis, a Herxheimer reaction to syphilis, a decompensated heart failure, allergic rhinitis or nasal polyps, comprising administering to a subject in need thereof a steroid payload or conjugate thereof described herein. In some examples, the compounds disclosed herein can be used for treating inflammatory bowel disease, Crohn's disease, or ulcerative colitis. In some examples, the disease, disorder, or condition is a chronic inflammatory condition including, but not limited to, asthma, skin infections, and ocular infections. In some examples, compounds described herein are used for immunosuppression in patients undergoing organ transplantation.

In some embodiments, the steroid payloads and conjugates thereof described herein are administered to a patient to treat a nervous disorder associated with GR signalling including, but not limited to, psychiatric disorders such as schizophrenia, drug addiction, post-traumatic stress disorder (PTSD), and mood disorders, substance abuse, stress, and anxiety. In some embodiments, the steroid payloads and conjugates thereof described herein are administered to a patient to treat a visual system disorder including, but not limited to, ocular inflammation (e.g., conjunctivitis, keratitis, uveitis), macular edema, and macular degeneration. In some embodiments, the steroid payloads and conjugates thereof described herein are administered to a patient to treat a cardiovascular disorder. In some embodiments, the steroid payloads and conjugates thereof described herein are administered to a patient to treat a glucose and/or liver metabolism disorder. In some embodiments, the steroid payloads and conjugates thereof described herein are administered to a patient to treat a musculoskeletal system disorder. In some embodiments, the steroid payloads and conjugates thereof described herein are administered to a patient to treat a cutaneous inflammatory condition, such as eczema and psoriasis.

The protein conjugates described herein provide a means for targeted delivery of its steroid payload to particular cells or organ systems, thereby reducing or preventing side effects that result from administration of the free unconjugated steroid payload. Examples of such potential side effects to be reduced or prevented include those listed in the approved drug label for Decadron® (dexamethasome), which is incorporated herein by reference in its entirety. In some embodiments, the side effect to be reduced or prevented is selected from elevation of blood pressure; sodium retention; water/fluid retention (edema, angioedema, pulmonary edema); increased excretion of potassium; reversible hypothalamic-pituitary adrenal (HPA) axis suppression; potential corticosteroid insufficiency after withdrawal of treatment; susceptibility to infecctions; exacerbation of systemic fungal infections; worsening of severity of chickenpox in pediatric and adult patients; worsening of severity of measles in pediatric and adult patients; posterior subcapsular cataracts; glaucoma with possible damage to the optic nerves; enhancement of the establishment of secondary ocular infections due to bacteria, fungi, or viruses; increase in new episodes of optic neuritis; Kaposi's sarcoma; drug-induced secondary adrenocortical insufficiency; increased risk of a perforation when active or latent peptic ulcers, diverticulitis, fresh intestinal anastomoses, and nonspecific ulcerative colitis, are present; peritoneal irritation following gastrointestinal perforation; decreased bone formation; increased bone resorption; inhibition of osteoblast function; inhibition of bone growth in pediatric patients; development of osteoporosis at any age; acute myopathy (possibly involving ocular and respiratory muscles, and potentially resulting in quadriparesis); elevation of creatinine kinase; psychic derangements, ranging from euphoria, insomnia, mood swings, personality changes, and severe depression, to frank psychotic manifestations; aggravation of existing emotional instability or psychotic tendencies; elevated intraocular pressure; bradycardia; cardiac arrest; cardiac arrhythmias; cardiac enlargement; circulatory collapse; congestive heart failure; fat embolism; hypertension; hypertrophic cardiomyopathy in premature infants; myocardial rupture following recent myocardial infarction; syncope; tachycardia; thromboembolism; thrombophlebitis; vasculitis; acne; allergic dermatitis; dry scaly skin; ecchymoses and petechiae; erythema; impaired wound healing; increased sweating; rash; striae; suppression of reactions to skin tests; thin fragile skin; thinning scalp hair; urticarial; decreased carbohydrate and glucose tolerance; development of cushingoid state; hyperglycemia; glycosuria; hirsutism; hypertrichosis; increased requirements for insulin or oral hypoglycemic agents in diabetes (insulin resistance); manifestations of latent diabetes mellitus; menstrual irregularities; secondary adrenocortical and pituitary unresponsiveness (particularly in times of stress; as in trauma; surgery; or illness); suppression of growth in pediatric patients; congestive heart failure in susceptible patients; fluid retention; hypokalemic alkalosis; potassium loss; sodium retention; abdominal distention; elevation in serum liver enzyme levels (usually reversible upon discontinuation); hepatomegaly; increased appetite; nausea; pancreatitis; peptic ulcer with possible perforation and hemorrhage; perforation of the small and large intestine (particularly in patients with inflammatory bowel disease); ulcerative esophagitis; negative nitrogen balance due to protein catabolism; aseptic necrosis of femoral and humeral heads; loss of muscle mass; muscle weakness; osteoporosis; pathologic fracture of long bones; steroid myopathy; tendon rupture; vertebral compression fractures; convulsions; depression; emotional instability; euphoria; headache; increased intracranial pressure with papilledema (pseudotumor cerebri) usually following discontinuation of treatment; insomnia; mood swings; neuritis; neuropathy; paresthesia; personality changes; psychic disorders; vertigo; exophthalmos; glaucoma; increased intraocular pressure; posterior subcapsular cataracts; abnormal fat deposits; decreased resistance to infection; hiccups; increased or decreased motility and number of spermatozoa; malaise; moon face; and weight gain; and those side effects associated with drug-drug interactions. In some embodiments, the side effect to be reduced or prevented are those associated with drug-drug interactions. In some embodiments, the side effect to be reduced or prevented is associated with drug-drug interactions from the use of a corticosteroid with aminoglutethimide including diminishment of adrenal suppression by corticosteroids; amphotericin B injection and potassium-depleting agents, including development of hypokalemia, cardiac enlargement, and congestive heart failure; antibiotics including a significant decrease in corticosteroid clearance; anticholinesterases including producing severe weakness in patients with myasthenia gravis; oral anticoagulants including inhibition of response to warfarin; antidiabetics including increased blood glucose concentrations; antitubercular drugs including decreased serum concentrations of isoniazid; cholestyramine including increased clearance of corticosteroids; cyclosporine including increased activity of both cyclosporine and corticosteroids, and incidence of convulsions; dexamethasone suppression test (DST) interference including false-negative results in patients being treated with indomethacin; digitalis glycosides including increased risk of arrhythmias due to hypokalemia; ephedrine including enhancement of the metabolic clearance of corticosteroids, resulting in decreased blood levels and lessened physiologic activity; estrogens, including oral contraceptives, including decreased hepatic metabolism of certain corticosteroids and associated increase in their effect; hepatic enzyme inducers, inhibitors and substrates (drugs which induce cytochrome P450 3A4 (CYP 3A4) enzyme activity e.g., barbiturates, phenytoin, carbamazepine, rifampin), including enhancing of metabolism of corticosteroids; drugs which inhibit CYP 3A4 (e.g., ketoconazole, macrolide antibiotics such as erythromycin), including the potential for increased plasma concentrations of corticosteroids; drugs that are metabolized by CYP 3A4 (e.g., indinavir, erythromycin), including increase in their clearance, resulting in decreased plasma concentration; ketoconazole including decreased metabolism of certain corticosteroids by up to 60%, leading to increased risk of corticosteroid side effects, and inhibition of adrenal corticosteroid synthesis potentially causing adrenal insufficiency during corticosteroid withdrawal; nonsteroidal anti-inflammatory agents (NSAIDS), including increased risk of gastrointestinal side effects and increased clearance of salicylates; phenytoin, including increases or decreases in phenytoin level, altered seizure control; skin tests, including suppression of reactions to skin tests; thalidomide including toxic epidermal necrolysis; and vaccines including a diminished response to toxoids and live or inactivated vaccines due to inhibition of antibody response or potentiation of the replication of some organisms contained in live attenuated vaccines). Thus, provided herein are methods for treating a disease, disorder, or condition associated with the glucocorticoid receptor comprising administering a conjugate of Formulae I, Ia, Iaa, Iaaa, Ib, Ibb, Ibbb, II, IIa, IIaa, IIaaa, IIb, IIbb, IIbbb, III, IIIa, IIIaa, IIIaaa, IIIb, IIIbb, IIIbbb, IV, and/or V, to a patient having said disease, disorder, or condition, wherein the side effects associated with administration of the free steroid payload of said conjugate is reduced. Furthermore, provided herein are methods of delivering a compound of Formulae I, Ia, Iaa, Iaaa, Ib, Ibb, Ibbb, II, IIa, IIaa, IIaaa, IIb, IIbb, IIbbb, III, IIIa, IIIaa, IIIaaa, Tub, IIIbb, IIIbbb, IV, and/or V, to a cell comprising contacting said cell with a protein conjugate the compound of Formulae I, Ia, Iaa, Iaaa, Ib, Ibb, Ibbb, II, IIa, IIaa, IIaaa, IIb, IIbb, IIbbb, III, IIIa, IIIaa, IIIaaa, IIIb, IIIbb, IIIbbb, IV, and/or V, wherein the protein conjugate comprises an antibody or antigen binding fragment thereof that binds a surface antigen of said cell.

In some examples, set forth herein is a method of treating a disease, disorder or condition selected from the group consisting of an immunological disease, autoimmune disease, inflammation, asthma, or an inflammatory bowel disorder, Crohn's disease, ulcerative colitis.

In some examples, set forth herein is a method of treating a disease, disorder or condition by targeting an antigen, e.g., cell-surface expressing antigen, to which steroid delivery can achieve a therapeutic benefit comprising administering the conjugates described herein. In some embodiments, the antigen is AXL, BAFFR, BCMA, BCR-list components, BDCA2, BDCA4, BTLA, BTNL2, BTNL3, BTNL8, BTNL9, C10orf54, CCR1, CCR3, CCR4, CCR5, CCR6, CCR7, CCR9, CCR10, CD11c, CD137, CD138, CD14, CD168, CD177, CD19, CD20, CD209, CD209L, CD22, CD226, CD248, CD25, CD27, CD274, CD276, CD28, CD30, CD300A, CD33, CD37, CD38, CD4, CD40, CD44, CD45, CD47, CD46, CD48, CD5, CD52, CD55, CD56, CD59, CD62E, CD68, CD69, CD70, CD74, CD79a, CD79b, CD8, CD80, CD86, CD90.2, CD96, CLEC12A, CLEC12B, CLEC7A, CLEC9A, CR1, CR3, CRTAM, CSF1R, CTLA4, CXCR½, CXCR4, CXCR5, DDR1, DDR2, DEC-205, DLL4, DR6, FAP, FCamR, FCMR, FcR's, Fire, GITR, HHLA2, HLA class II, HVEM, ICOSLG, IFNLR1, IL10R1, IL10R2, IL12R, IL13RA1, IL13RA2, IL15R, IL17RA, IL17RB, IL17RC, IL17RE, IL20R1, IL20R2, IL21R, IL22R1, IL22RA, IL23R, IL27R, IL29R, IL2Rg, IL31R, IL36R, IL3RA, IL4R, IL6R, IL5R, IL7R, IL9R, Integrins, LAG3, LIFR, MAG/Siglec-4, MMR, MSR1, NCR3LG1, NKG2D, NKp30, NKp46, PDCD1, PROKR1, PVR, PVRIG, PVRL2, PVRL3, RELT, SIGIRR, Siglec-1, Siglec-10, Siglec-5, Siglec-6, Siglec-7, Siglec-8, Siglec-9, SIRPA, SLAMF7, TACI, TCR-list components/assoc, PTCRA, TCRb, CD3z, CD3, TEK, TGFBR1, TGFBR2, TGFBR3, TIGIT, TLR2, TLR4, TROY, TSLPR, TYRO, VLDLR, VSIG4, or VTCN1. In some embodiments, the antigen is IL2R-γ.

In some examples, set forth herein is a method for treating a disease, disorder, or condition selected from an immunological disease, an autoimmune disease, an inflammatory disease, a dermatological disease, or a gastrointestinal disease.

In some examples, the disease is Crohn's disease, ulcerative colitis, Cushing's syndrome, adrenal insufficiency, or congenital adrenal hyperplasia.

In some examples, the disease is inflammation, asthma, or an inflammatory bowel disorder.

In some examples, the disease is an autoimmune diseases selected from multiple sclerosis, rheumatoid arthritis, inflammatory bowel disease, ulcerative colitis, psoriasis, or eczema

In some examples, set forth herein is a method for reducing or ameliorating the side effects of chemotherapy, wherein the method includes administering to a patient having said disorder a therapeutically effective amount of a compound or a composition described herein.

In some examples, set forth herein is a method for reducing or ameliorating the side effects of immunosuppressive therapy, wherein the method includes administering to a patient having said disorder a therapeutically effective amount of a compound or a composition described herein.

In some examples, set forth herein is a method for treating cancer, wherein the method includes administering to a patient having said disorder a therapeutically effective amount of a compound or a composition described herein. In some examples, the cancer is selected from acute lymphoblastic leukemia, chronic lymphoblastic leukemia, Hodgkin's lymphoma, Non-Hodgkin's lymphoma (NHL), or multiple myeloma, as well as others.

In some examples, set forth herein are methods for treating or preventing any disease, disorder, or condition responsive to modulation of LXR signalling. In some examples, the disease or disorder is associated with LXR function, LXR polymorphisms, LXR agonist activity, or LXR antagonist activity. In some examples, set forth herein is a method of treating or preventing a disease, disorder, or condition selected from the group consisting of a proliferative disorder, a neurodegenerative disorder, an immunological disorder, an autoimmune disease, an inflammatory disorder, a dermatological disease, a metabolic disease, cardiovascular disease, and a gastrointestinal disease.

The proliferative disorder can be any proliferative disorder known to those of skill. In certain embodiments, proliferative disorders include, without limitation, oncology disorders, where the oncology disorder can be any cancer disorder known to those of skill. In certain embodiments, provided herein are methods of treating or preventing a melanoma. In certain embodiments, provided herein are methods of treating or preventing metastatic melanoma. In certain embodiments, provided herein are methods of treating or preventing lung cancer. In certain embodiments, provided herein are methods of treating or preventing EGFR-tyrosine kinase inhibitor resistant lung cancer. In certain embodiments, provided herein are methods of treating or preventing oral cancer. In certain embodiments, provided herein are methods of treating or preventing oral squamous cell carcinoma. In certain embodiments, provided herein are methods of treating or preventing prostate cancer. In certain embodiments, provided herein are methods of treating or preventing Hodgkin's lymphoma. In certain embodiments, provided herein are methods of treating or preventing breast cancer.

The neurodegenerative disorder can be any neurodegenerative disorder known to those of skill. In certain embodiments, provided herein are methods of treating or preventing Alzheimer's disease. In certain embodiments, provided herein are methods of treating or preventing Parkinson's disease. In certain embodiments, provided herein are methods of treating or preventing Huntington's disease. In certain embodiments, provided herein are methods of treating or preventing amyotrophic lateral sclerosis. In certain embodiments, provided herein are methods of treating or preventing myelin gene expression. In certain embodiments, provided herein are methods of treating or preventing myelination and remyelination conditions, diseases, or disorders.

The immunological disorder can be any immunological disorder known to those of skill. In certain embodiments, provided herein are methods of treating or preventing inflammatory bowel disease. In certain embodiments, provided herein are methods of treating or preventing ulcerative colitis. In certain embodiments, provided herein are methods of treating or preventing Crohn's disease.

The inflammatory disorder can be any inflammatory disorder known to those of skill. In certain embodiments, provided herein are methods of treating or preventing arthritis. In certain embodiments, provided herein are methods of treating or preventing rheumatoid arthritis.

The metabolic disease can be any metabolic disease known to those of skill. In certain embodiments, the metabolic disease is dyslipidemia. Dyslipidemia can be any dyslipidemia known to those of skill. In certain embodiments, dyslipidemia is selected from the group consisting of hyperlipidemia, hypercholesterolemia, hypertriglyceridemia, hyperlipoproteinemia, HDL deficiency, ApoA-I deficiency, and cardiovascular disease such as coronary artery disease (including, for example, treatment and prevention of angina, myocardial infarction, and sudden cardiac death); atherosclerosis (including, for example, treatment and prevention of atherosclerosis); and restenosis (including, for example, preventing or treating atherosclerotic plaques which develop as a consequence of medical procedures such as balloon angioplasty). In certain embodiments, provided herein are methods of treating or preventing diabetes.

The cardiovascular disease can be any cardiovascular disease known to those of skill. In certain embodiments, provided herein are methods of treating or preventing atherosclerosis. In certain embodiments, provided herein are methods of treating or preventing atherosclerosis derived from abnormal macrophage processing. In certain embodiments, provided herein are methods of treating or preventing atherosclerosis derived from the formation of oxidized low-density lipoproteins (oxLDLs), where marcrophages fail to process oxLDLs. In certain embodiments, provided herein are methods of treating or preventing ischemic heart disease. In certain embodiments, provided herein are methods of treating or preventing stroke. In certain embodiments, provided herein are methods of treating or preventing hypertensive heart disease. In certain embodiments, provided herein are methods of treating or preventing aortic aneurysm. In certain embodiments, provided herein are methods of treating or preventing endocarditis. In certain embodiments, provided herein are methods of treating or preventing peripheral artery disease. In certain embodiments, provided herein are methods of treating or preventing combinations of any of the diseases provided in this paragraph.

In some examples, set forth herein is a method for modulating the function of a nuclear receptor. By way of non-limiting example, the function may be selected from expression/secretion of inflammatory mediators (e.g. cytokines, chemokines), cholesterol regulation, cholesterol intake, cholesterol efflux, cholesterol oxidation, migration, chemotaxis, apoptosis and necrosis, an inflammatory activity, lipid regulation, apoptosis, migration, chemotaxis, gene transcription, and protein expression.

### EXAMPLES

Provided herein are novel steroid and LXR modulator compounds, prodrugs, or payloads, protein conjugates thereof, and methods for treating diseases, disorders, and conditions including administering the steroid and LXR modulator compounds, prodrugs, or payloads and conjugates.

TABLE 1
List of Hydroxyl-Glucocorticoid Receptor Agonists

| | Drug/Payload | | |
|---|---|---|---|
| Drug#/Payload | Name | Structure | cLogP |
| I | Dexamethasone | 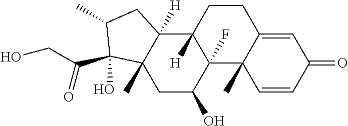 | 1.68 |
| II | Budesonide | 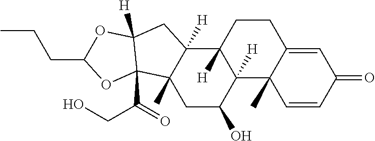 | 2.73 |
| III | 6,11-2F- Budesonide | 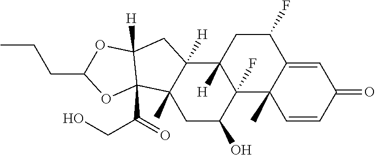 | 2.44 |
| IV | LXR Agonist | 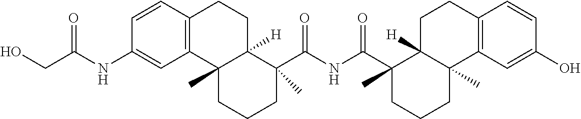 [SMILES: C[C@]1(C(=O)NC(=O)[C@@]2(C)CCC[C@]3(C)c4cc(NC(=O)CO)ccc4CC[C@@H]23)CCC[C@]2(C)c3cc(O)ccc3CC[C@@H]12] | 6.53 |

TABLE 2a
List of N—C—O-Steroid Prodrugs

| Cpd | | | | | | |
|---|---|---|---|---|---|---|
| No. | n | R² | R³ | R¹ | D*—OH | Structure |
| PI | 1 | H | H | H | I (Dex) | 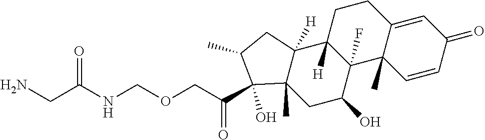 |
| PII-1 | 1 | H | H | H | II (Bud) | 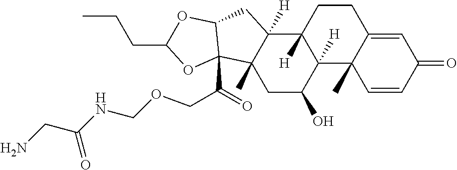 |
| PII-2 | 1 | Me | H | H | II (Bud) | 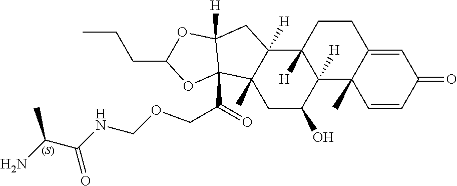 |
| PII-3 | 1 | Bn | H | H | II (Bud) | 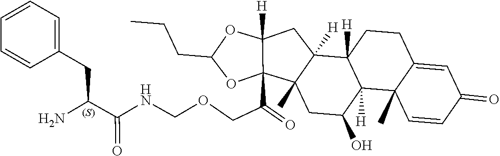 |
| PII-4 | 2 | H | H | H | II (Bud) | 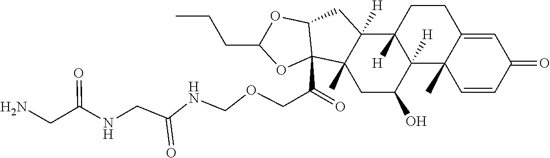 |
| PII-5 | 2 | Me | H | H | II (Bud) | 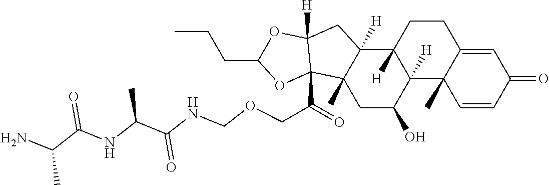 |
| PII-6 | 1 | H | H | Et | II (Bud) | 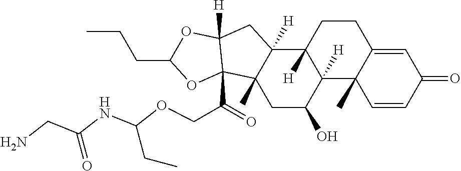 |
| PII-7 | 1 | H | H | Bn | II (Bud) | 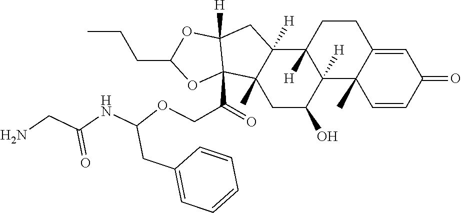 |
| PII-8 | 1 | H | Me | H | II (Bud) | 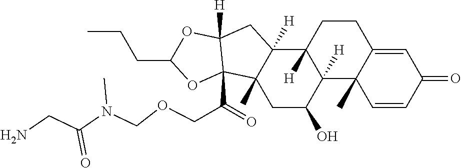 |
| PII-9 | 1 | H | 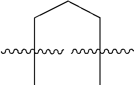 [SMILES: *CCC*] | | II (Bud) | 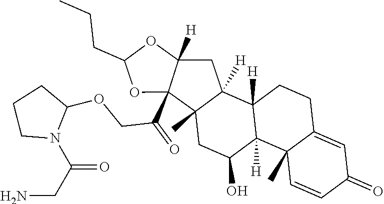 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CN)O1] |
| PII-10 | 1 | H | 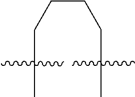 [SMILES: *CCCC*] | | II (Bud) | 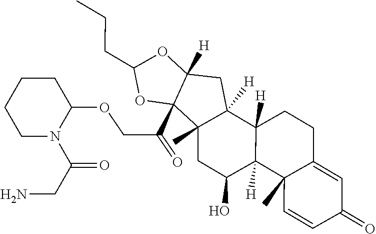 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCCN2C(=O)CN)O1] |
| PIII-1 | 1 | H | H | H | III | 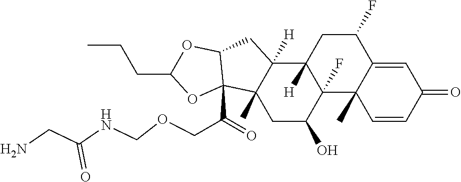 |
| PIII-4 | 2 | H | H | H | III | 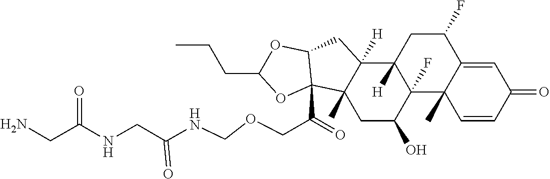 |
| PII-11 | 1 | Me | Me | H | II (Bud) | 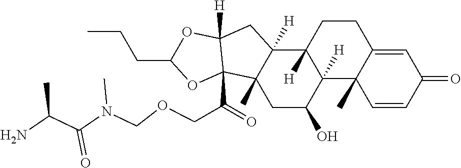 |
| PII-12 | 1 | 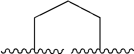 [SMILES: *CCC*] | | H | II (Bud) | 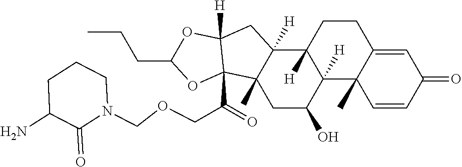 |
| PII-13 | 1 | H | 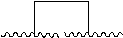 [SMILES: *CC*] | | II (Bud) | 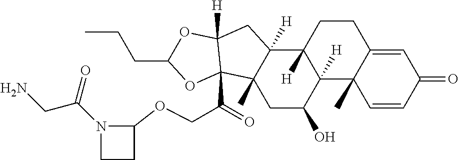 |
| PIII-5 | 1 | Me | H | H | III | 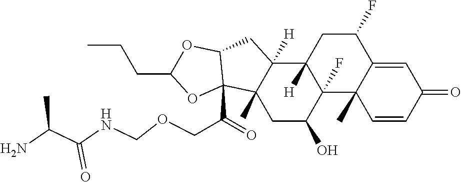 |
| PIV | 1 | H | H | H | IV | 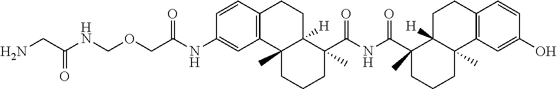 |

TABLE 2b
Properties of N—C—O-Steroid Prodrugs

| | | | | | | | | | HPLC | |
|---|---|---|---|---|---|---|---|---|---|---|
| Cpd No. | n | R² | R³ | R¹ | D*—OH | cLogP | MF | MW | Purity (%) | Rt (min) |
| PI | 1 | H | H | H | I (Dex) | 0.53 | C₂₅H₃₅FN₂O₆ | 478.6 | 99 | 5.42 (A) |
| PII-1 | 1 | H | H | H | II (Bud) | 1.58 | C₂₈H₄₀N₂O₇ | 516.6 | 96 | 7.55 (B) |
| PII-2 | 1 | Me | H | H | II (Bud) | 2.15 | C₂₉H₄₂N₂O₇ | 530.7 | 96 | 7.40 (B) |
| PII-3 | 1 | Bn | H | H | II (Bud) | 3.81 | C₃₅H₄₆N₂O₇ | 606.8 | 99 | 8.39 (B) |
| PII-4 | 2 | H | H | H | II (Bud) | 0.48 | C3₀H₄₃N₃O₈ | 573.7 | 98 | 6.90 (B) |
| PII-5 | 2 | Me | H | H | II (Bud) | 1.62 | C₃₂H₄₇N₃O₈ | 601.7 | >99 | 7.25 (B) |
| PII-6 | 1 | H | H | Et | II (Bud) | 2.45 | C₃₀H₄₄N₂O₇ | 544.7 | 95 | 7.96, |
| | | | | | | | | | | 8.15 (B) |
| PII-7 | 1 | H | H | Bn | II (Bud) | 3.59 | C₃₅H₄₆N₂O₇ | 606.7 | 99 | 8.49, |
| | | | | | | | | | | 8.66 (B) |
| PII-8 | 1 | H | Me | H | II (Bud) | 1.81 | C₂₉H₄₂N₂O₇ | 530.7 | >99 | 7.42 (B) |
| PII-9 | 1 | H | 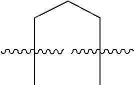 [SMILES: *CCC*] | | II (Bud) | 2.20 | C₃₁H₄₄N₂O₇ | 556.7 | >99 | 7.87 (B) |
| PII-10 | 1 | H | 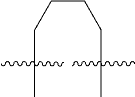 [SMILES: *CCCC*] | | II (Bud) | 2.65 | C₃₂H₄₆N₂O₇ | 570.7 | | |
| PIII-1 | 1 | H | H | H | III | 1.29 | C₂₈H₃₈F₂N₂O₇ | 552.6 | 99 | 6.97 (B) |
| PIII-4 | 2 | H | H | H | III | 0.18 | C₃₀H₄₁F₂N₃O₈ | 609.7 | 99 | 6.95 (B) |
| PII-11 | 1 | Me | Me | H | II (Bud) | 2.38 | C₃₀H₄₄N₂O₇ | 544.7 | >99 | 7.62 (B) |
| PII-12 | 1 | 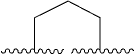 [SMILES: *CCC*] | | H | II (Bud) | 2.43 | C₃₁H₄₄N₂O₇ | 556.7 | >99 | 8.05, 8.12 (B) |
| PII-13 | 1 | H | 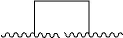 [SMILES: *CC*] | | II (Bud) | 2.65 | C₃₂H₄₆N₂O₇ | 570.7 | 99 | 9.93 (B) |
| PIII-5 | 1 | Me | H | H | III | 1.86 | C₂₉H₄₀F₂N₂O₇ | 566.6 | | |
| PIV | 1 | H | H | H | IV | 5.25 | C₃₉H₅₂N₄O₆ | 672.9 | 99 | 7.83 (B) |

TABLE 3a
List of Linker-N—C—O-Steroid Prodrugs

| Cpd# | P | Name | Structures |
|---|---|---|---|
| L1- PI | PI | DIBAC- suc- PEG4- vcPAB- PI | 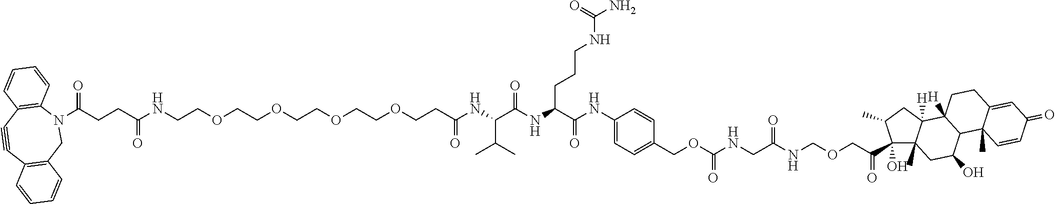 [SMILES: CC(C)[C@H](NC(=O)CCOCCOCCOCCOCCNC(=O)CCC(=O)N1Cc2ccccc2C#Cc2ccccc21)C(=O)N[C@@H](CCCNC(N)=O)C(=O)Nc1ccc(COC(=O)NCC(=O)NCOCC(=O)[C@@]2(O)[C@H](C)C[C@H]3[C@@H]4CCC5=CC(=O)C=C[C@]5(C)C4[C@@H](O)C[C@@]32C)cc1] |
| L1- PII- 1 | PII- 1 | DIBAC- suc- PEG4- vcPAB- PII-1 | 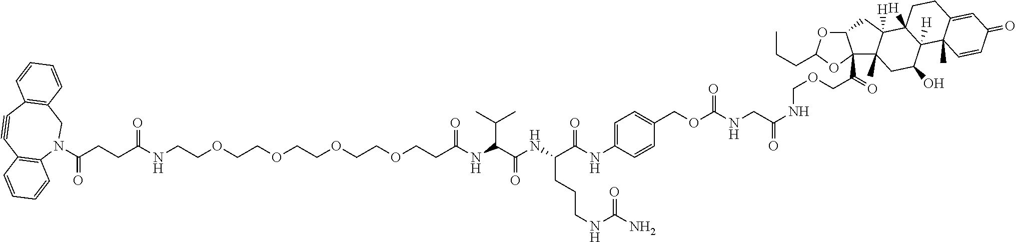 |
| L1- PII- 2 | PII- 2 | DIBAC- suc- PEG4- vcPAB- PII-2 | 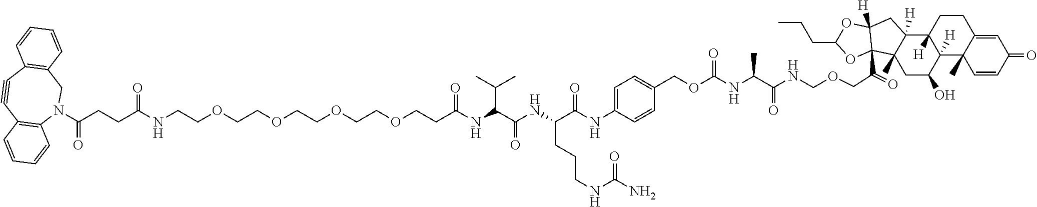 |
| L1- PII- 3 | PII- 3 | DIBAC- PEG₄- vcPAB- F- NHCH₂- Bud | 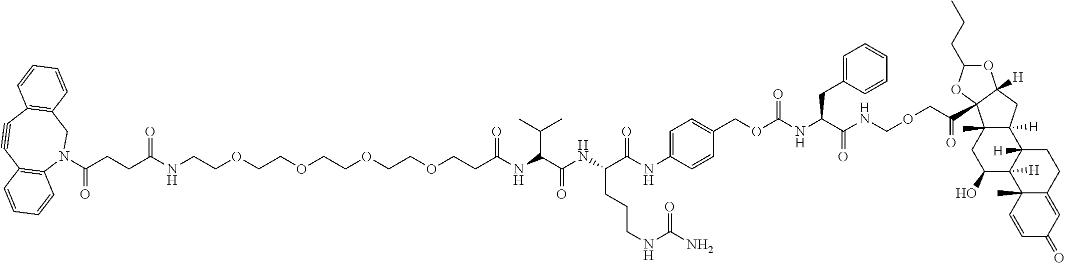 |
| Ll- PII- 4 | PII- 4 | DIBAC- suc- PEG4- vcPAB- PII-4 | 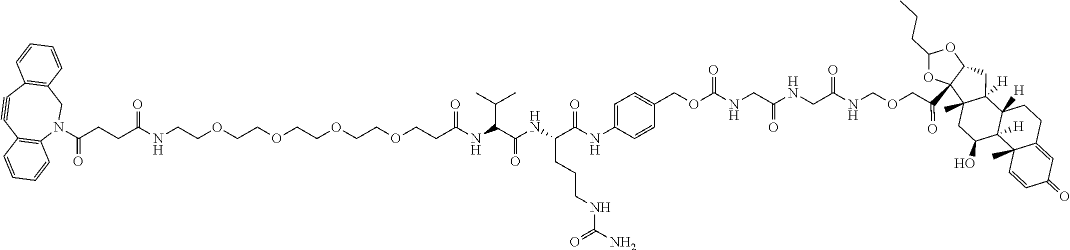 |
| L1- PII- 5 | PII- 5 | DIBAC- suc- PEG4- vcPAB- PII-5 | 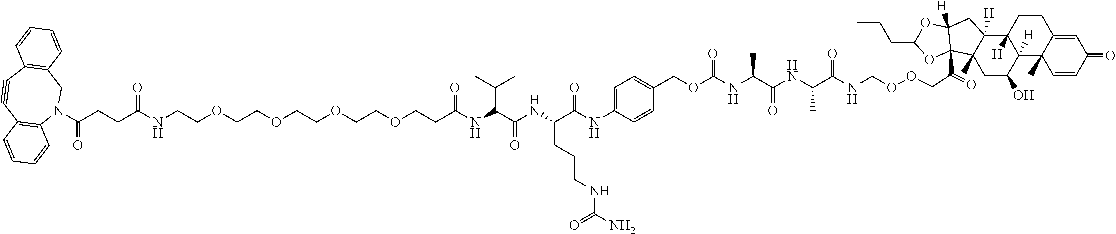 |
| L1- PII- 6 | PII- 6 | DIBAC- suc- PEG4- vcPAB- PII-6 | 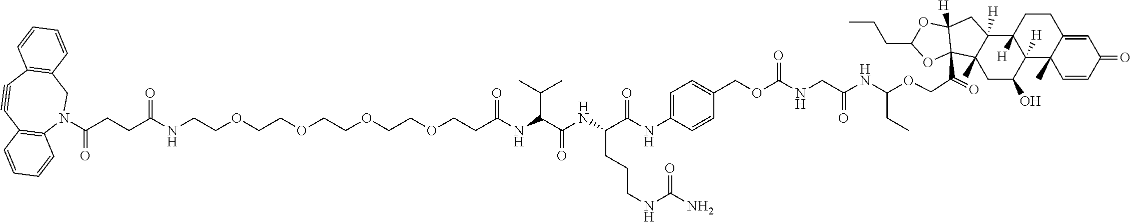 |
| L1- PII- 7 | PII- 7 | DIBAC- suc- PEG4- vcPAB- PII-7 | 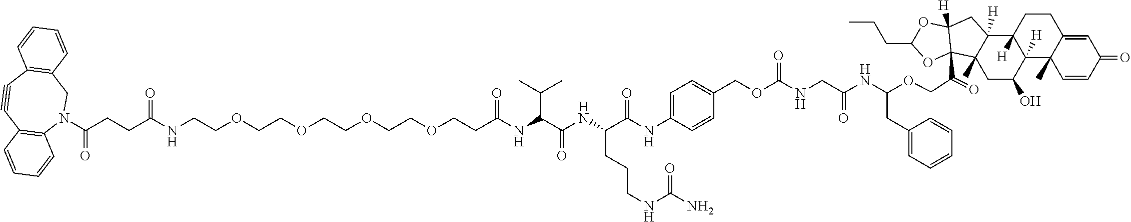 |
| L1- PII- 8 | PII- 8 | DIBAC- suc- PEG4- vcPAB- PII-8 | 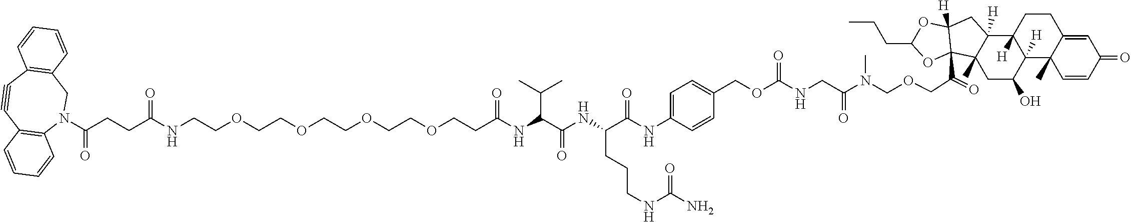 |
| L1- PII- 9 | PII- 9 | DIBAC- suc- PEG4- vcPAB- PII-9 | 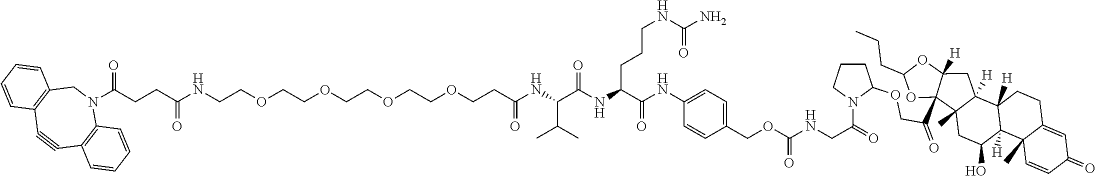 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)O1] |
| Ll- PII- 10 | PII- 10 | DIBAC- suc- PEG4- vcPAB- PII-10 | 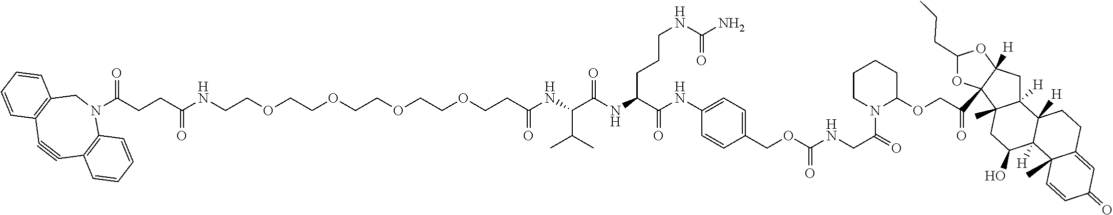 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCCN2C(=O)CNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)O1] |
| Ll- PIII- 1 | PIII- 1 | DIBAC- suc- PEG4- vcPAB- PIII-1 | 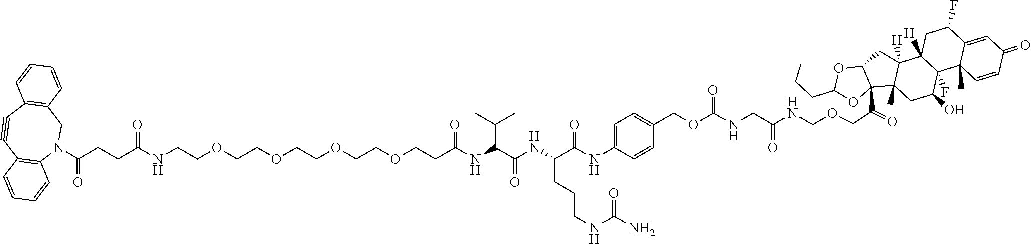 |
| L2- PIII- 1 | PIII- 1 | DIBAC- suc- PEG4- Glu- vcPAB- PIII-1 | 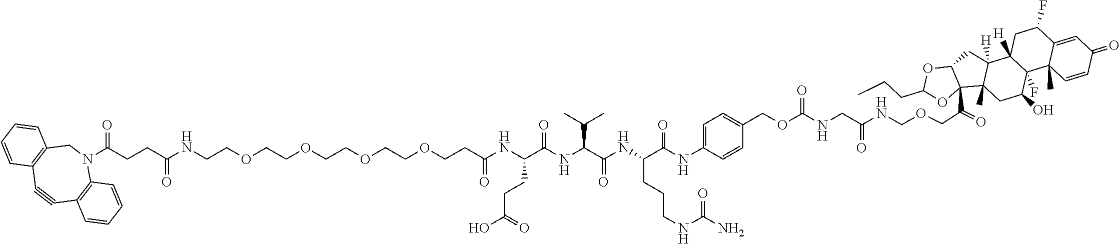 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)OCc2ccc(NC(=O)[C@H](CCCNC(N)=O)NC(=O)[C@@H](NC(=O)[C@H](CCC(=O)O)NC(=O)CCOCCOCCOCCOCCNC(=O)CCC(=O)N3Cc4ccccc4C#Cc4ccccc43)C(C)C)cc2)O1] |
| L3- PIII- 1 | PIII- 1 | DIBAC- suc- PEG4- GGF- PIII-1 | 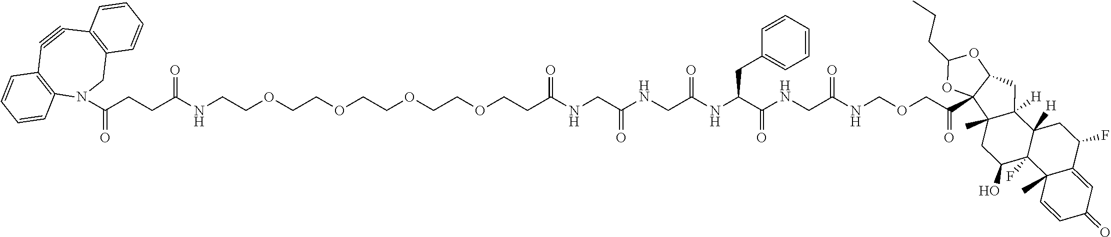 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)[C@H](Cc2ccccc2)NC(=O)CNC(=O)CNC(=O)CCOCCOCCOCCOCCNC(=O)CCC(=O)N2Cc3ccccc3C#Cc3ccccc32)O1] |
| L4- PIII- 1 | PIII- 1 | DIBAC- suc- GGF- PIII-1 | 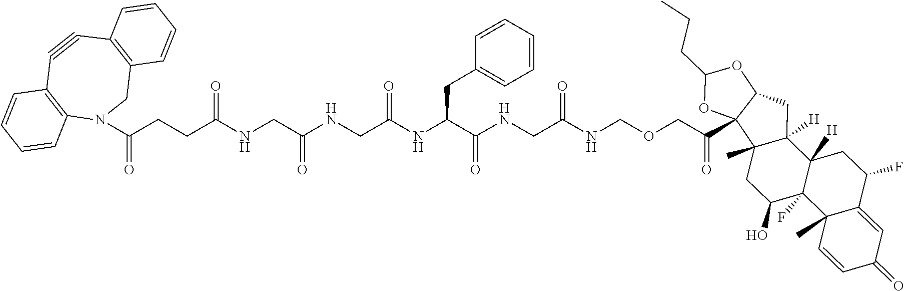 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)[C@H](Cc2ccccc2)NC(=O)CNC(=O)CNC(=O)CCC(=O)N2Cc3ccccc3C#Cc3ccccc32)O1] |
| L5- PIII- 1 | PIII- 1 | DIBAC- suc- SGGG- PIII-1 | 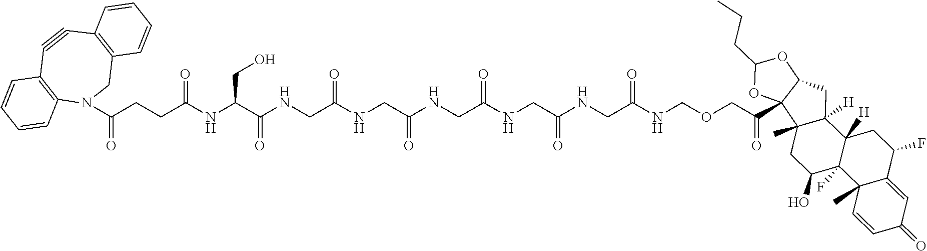 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)CNC(=O)CNC(=O)CNC(=O)CNC(=O)[C@H](CO)NC(=O)CCC(=O)N2Cc3ccccc3C#Cc3ccccc32)O1] |
| Ll- PIII- 4 | PIII- 4 | DIBAC- suc- PEG4- vcPAB- PIII-4 | 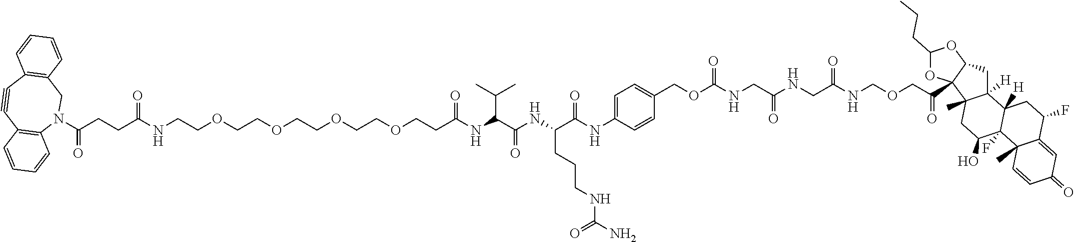 |
| L6- PII- 9 | PII- 9 | DIBAC- suc- PEG4- PII-9 | 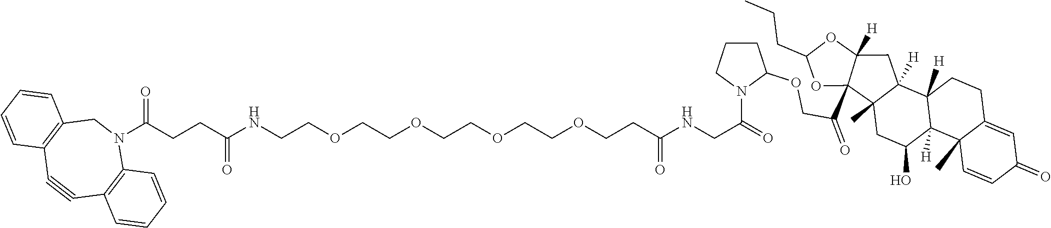 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CNC(=O)CCOCCOCCOCCOCCNC(=O)CCC(=O)N2Cc3ccccc3C#Cc3ccccc32)O1] |
| L7- PII- 1 | PII- 1 | DIBAC- suc- PII-1 | 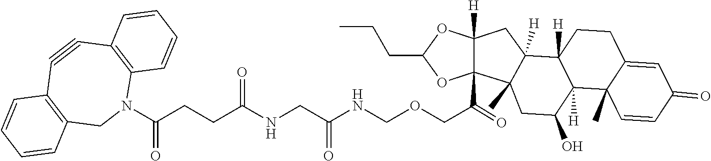 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CCC(=O)N2Cc3ccccc3C#Cc3ccccc32)O1] |
| L4- PI | PI | DIBAC- GGFG- NHCH₂- Dex | 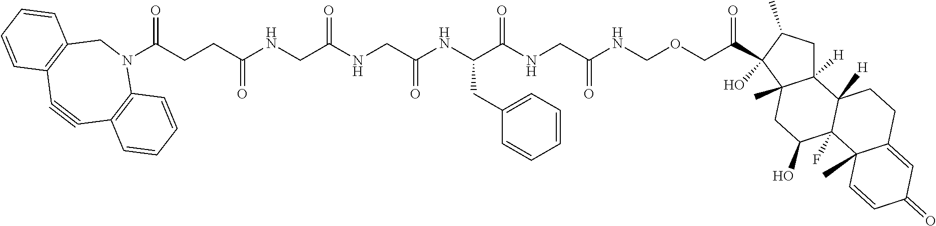 [SMILES: C[C@@H]1C[C@H]2[C@@H]3CCC4=CC(=O)C=C[C@]4(C)[C@@]3(F)[C@@H](O)C[C@]2(C)[C@@]1(O)C(=O)COCNC(=O)CNC(=O)[C@H](Cc1ccccc1)NC(=O)CNC(=O)CNC(=O)CCC(=O)N1Cc2ccccc2C#Cc2ccccc21] |
| L1- PII- 11 | PII- 11 | DIBAC- PEG₄- vcPAB- A- NMeCH₂- Bud | 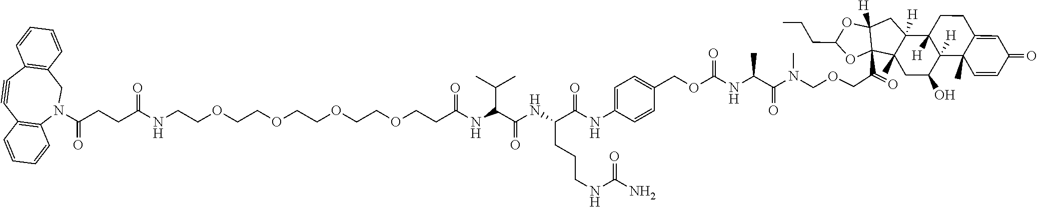 |
| L1- PII- 12 | PII- 12 | DIBAC- PEG₄- vcPAB- amino- piper- idi- none- CH₂- Bud | 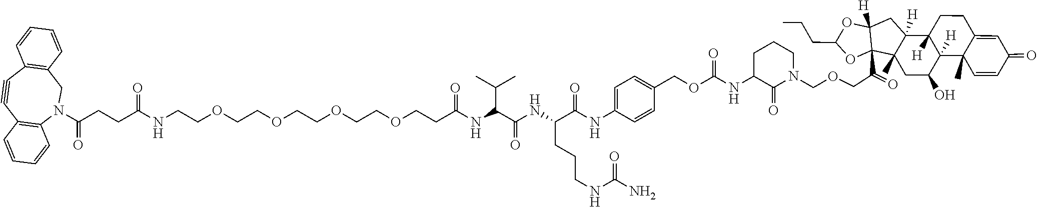 |
| L8- PII- 1 | PII- 1 | COT- PEG₄- EvcPAB- G- NHCH₂- Bud | 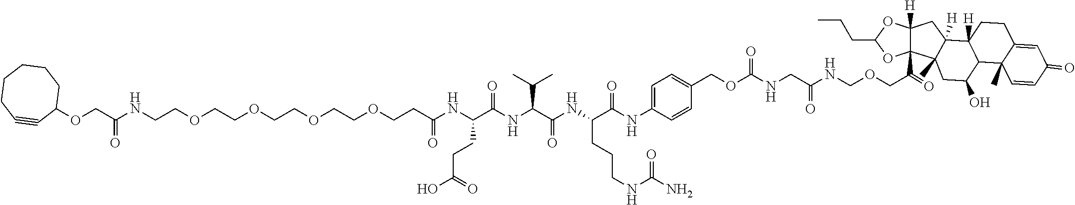 [SMILES: CCCC1O[C@@H]2C[C@H]3[C@@H]4CCC5=CC(=O)C=C[C@]5(C)C4[C@@H](O)C[C@]3(C)[C@]2(C(=O)COCNC(=O)CNC(=O)OCc2ccc(NC(=O)[C@H](CCCNC(N)=O)NC(=O)[C@@H](NC(=O)[C@H](CCC(=O)O)NC(=O)CCOCCOCCOCCOCCNC(=O)COC3C#CCCCCC3)C(C)C)cc2)O1] |
| L9- PII- 1 | PII- 1 | COT- GG- NHCH₂- Bud | 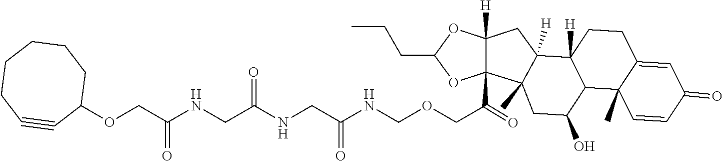 [SMILES: CCCC1O[C@@H]2C[C@H]3[C@@H]4CCC5=CC(=O)C=C[C@]5(C)C4[C@@H](O)C[C@]3(C)[C@]2(C(=O)COCNC(=O)CNC(=O)CNC(=O)COC2C#CCCCCC2)O1] |
| L10- PII- 1 | PII- 1 | COT- GGG- NHCH₂- Bud | 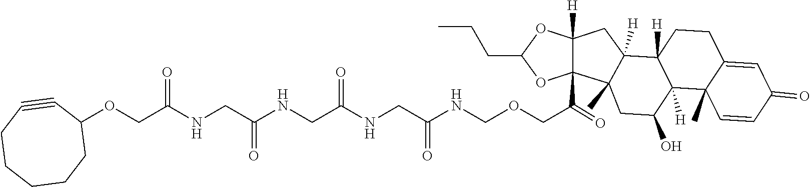 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CNC(=O)CNC(=O)COC2C#CCCCCC2)O1] |
| L11- PII- 2 | PII- 2 | COT- AAA- NHCH₂- Bud | 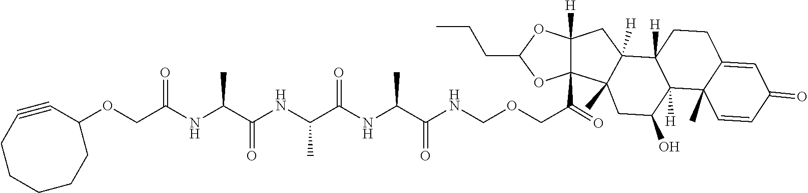 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)[C@H](C)NC(=O)[C@H](C)NC(=O)[C@H](C)NC(=O)COC2C#CCCCCC2)O1] |
| L12- PII- 2 | PII- 2 | COT- A^{d}AA- NHCH₂- Bud | 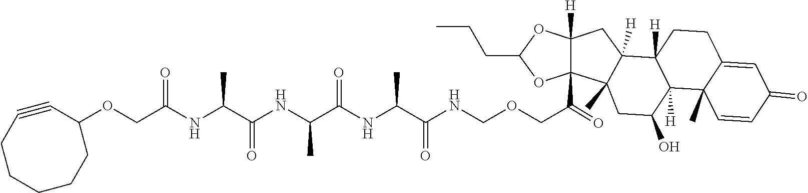 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)[C@H](C)NC(=O)[C@@H](C)NC(=O)[C@H](C)NC(=O)COC2C#CCCCCC2)O1] |
| L13- PII- 1 | PII- 1 | COT- GGGG- NHCH₂- Bud | 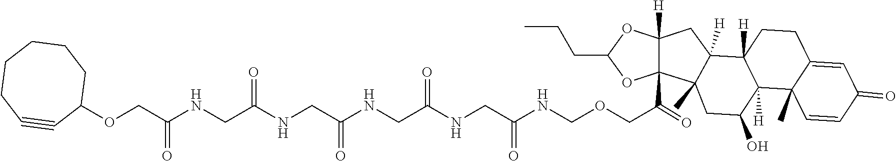 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CNC(=O)CNC(=O)CNC(=O)COC2C#CCCCCC2)O1] |
| L14- PII- 1 | PII- 1 | DIBAC- GGFG- NHCH₂- Bud | 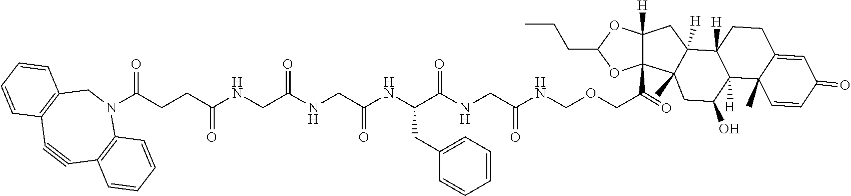 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)[C@H](Cc2ccccc2)NC(=O)CNC(=O)CNC(=O)CCC(=O)N2Cc3ccccc3C#Cc3ccccc32)O1] |
| L15- PII- 1 | PII- 1 | COT- SGGGG- NHCH₂- Bud | 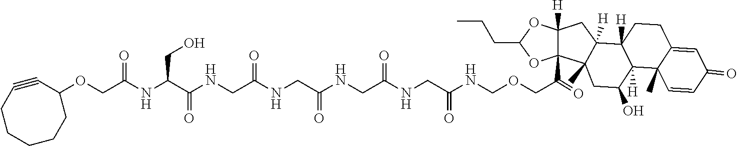 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CNC(=O)CNC(=O)CNC(=O)[C@H](CO)NC(=O)COC2C#CCCCCC2)O1] |
| L16- PII- 1 | PII- 1 | COT- KGGGG- NHCH₂- Bud | 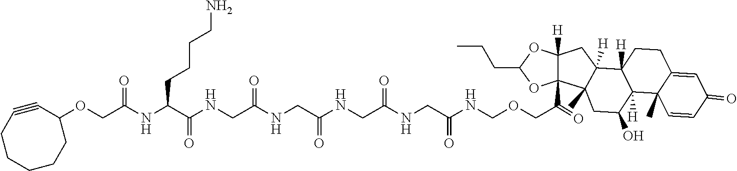 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CNC(=O)CNC(=O)CNC(=O)[C@H](CCCCN)NC(=O)COC2C#CCCCCC2)O1] |
| L17- PII- 1 | PII- 1 | COT- PEG₄- G- NHCH₂- Bud | 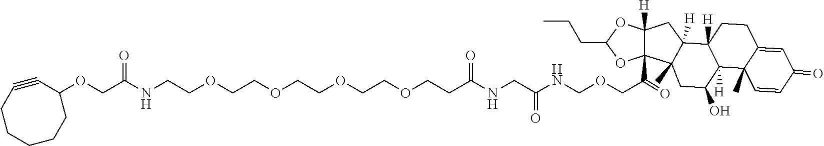 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CCOCCOCCOCCOCCNC(=O)COC2C#CCCCCC2)O1] |
| L6- PII- 13 | PII- 13 | DIBAC- PEG₄- G- Azeti- dine- Bud | 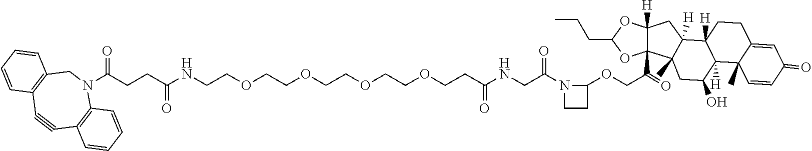 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCN2C(=O)CNC(=O)CCOCCOCCOCCOCCNC(=O)CCC(=O)N2Cc3ccccc3C#Cc3ccccc32)O1] |
| L18- PII- 1 | PII- 1 | COT- EDA- (GLC)PAB- G-NHCH₂- Bud | 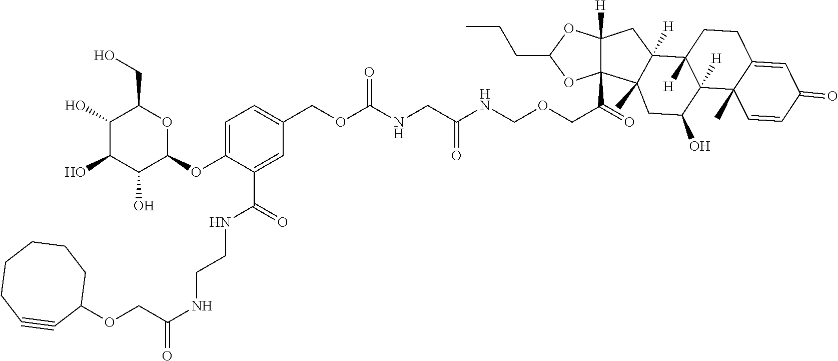 |
| L19- PIII- 1 | PIII- 1 | BCN- PEG₄- vcPAB- G- NHCH₂ - III | 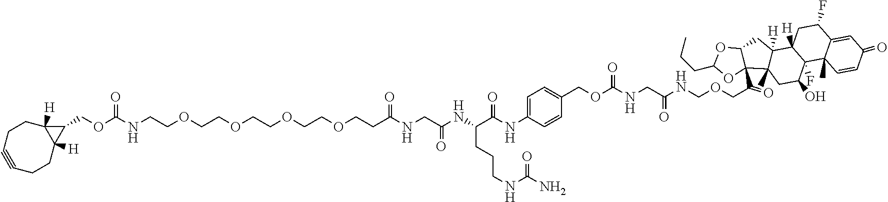 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)OCc2ccc(NC(=O)[C@H](CCCNC(N)=O)NC(=O)CNC(=O)CCOCCOCCOCCOCCNC(=O)OC[C@@H]3[C@@H]4CCC#CCC[C@@H]43)cc2)O1] |
| L9- PIII- 1 | PIII- 1 | COT- GG- NHCH₂- III | 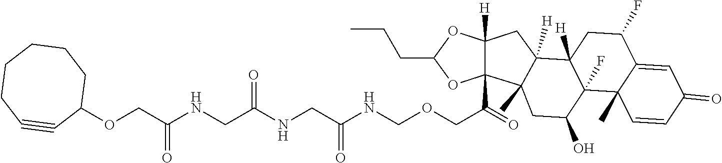 |
| L12- PIII- 5 | PIII- 5 | COT- A^{d}AA- NHCH₂- III | 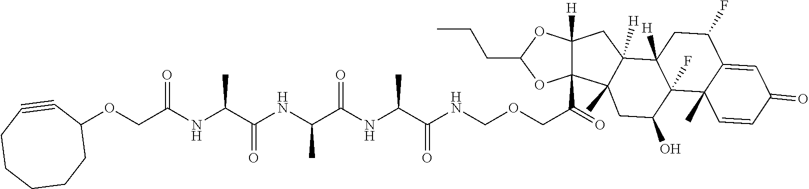 |
| L13- PIII- 1 | PIII- 1 | COT- GGGG- NHCH₂- III | 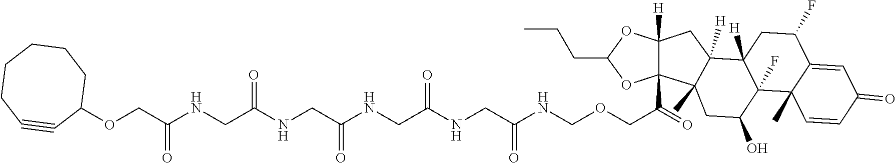 |
| L1- PIV | PIV | DIBAC- PEG₄- vcPAB- G- NHCH₂- IV | 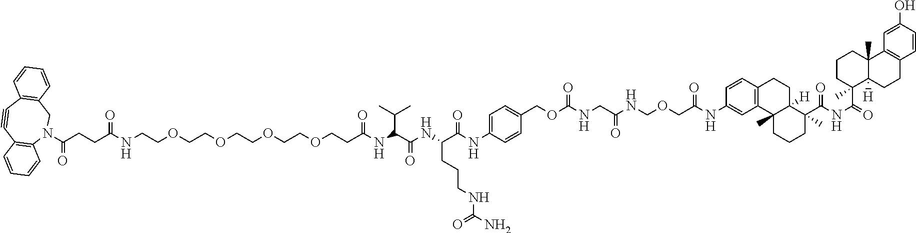 |
| L14- PIV | PIV | DIBAC- GGFG- NHCH₂- IV | 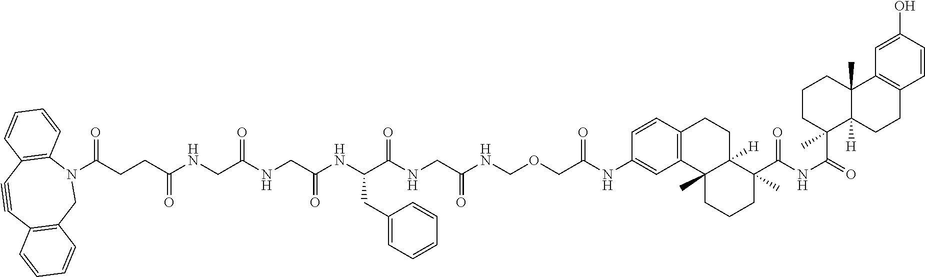 [SMILES: C[C@]1(C(=O)NC(=O)[C@@]2(C)CCC[C@]3(C)c4cc(NC(=O)COCNC(=O)CNC(=O)[C@H](Cc5ccccc5)NC(=O)CNC(=O)CNC(=O)CCC(=O)N5Cc6ccccc6C#Cc6ccccc65)ccc4CC[C@@H]23)CCC[C@]2(C)c3cc(O)ccc3CC[C@@H]12] |

TABLE 3b
Properties of Linker-N—C—O-Glucocorticoids

| | | | | | | HPLC | |
|---|---|---|---|---|---|---|---|
| | | | | | | Purity | Rt |
| Cpd# | P | Name | cLogP | MF | MW | (%) | (min)¹ |
| L1-PI | PI | DIBAC-suc-PEG4-vcPAB-PI | 2.60 | C₇₄H₉₆FN₉O₁₈ | 1418.6 | 97 | 7.71 |
| L1-PII-1 | PII-1 | DIBAC-suc-PEG4-vcPAB-PII-1 | 3.65 | C₇₇H₁₀₁N₉O₁₉ | 1456.7 | 96 | 8.07 |
| L1-PII-2 | PII-2 | DIBAC-suc-PEG4-vcPAB-PII-2 | 4.22 | C₇₈H₁₀₃N₉O₁₉ | 1470.7 | 95 | 8.27 |
| L1-PII-3 | PII-3 | DIBAC-suc-PEG4-vcPAB-PII-3 | 5.87 | C₈₄H₁₀₇N₉O₁₉ | 1546.8 | 96 | 8.70 |
| L1-PII-4 | PII-4 | DIBAC-suc-PEG4-vcPAB-PII-4 | 2.54 | C₇₉H₁₀₄N₁₀O₂₀ | 1513.8 | 98 | 7.94 |
| L1-PII-5 | PII-5 | DIBAC-suc-PEG4-vcPAB-PII-5 | 3.68 | C₈₁H₁₀₈N₁₀O₂₀ | 1541.8 | >99 | 8.15 |
| L1-PII-6 | PII-6 | DIBAC-suc-PEG4-vcPAB-PII-6 | 4.51 | C₇₉H₁₀₅N₉O₁₉ | 1484.8 | >99 | 8.61, 8.71 |
| L1-PII-7 | PII-7 | DIBAC-suc-PEG4-vcPAB-PII-7 | 5.65 | C₈₄H₁₀₇N₉O₁₉ | 1546.8 | 97 | 8.89, 8.96 |
| L1-PII-8 | PII-8 | DIBAC-suc-PEG4-vcPAB-PII-8 | 3.87 | C₇₈H₁₀₃N₉O₁₉ | 1470.7 | >99 | 8.41 |
| L1-PII-9 | PII-9 | DIBAC-suc-PEG4-vcPAB-PII-9 | 4.26 | C₈₀H₁₀₅N₉O₁₉ | 1496.8 | >99 | 8.78 |
| L1-PII-10 | PII-10 | DIBAC-suc-PEG4-vcPAB-PII-10 | 4.71 | C₈₁H₁₀₇N₉O₁₉ | 1510.8 | | |
| L1-PIII-1 | PIII-1 | DIBAC-suc-PEG4-vcPAB-PIII-1 | 3.35 | C₇₇H₉₉F₂N₉O₁₉ | 1492.7 | >99 | 7.99 |
| L2-PIII-1 | PIII-1 | DIBAC-suc-PEG4-Glu-vcPAB-PIII-1 | 1.98 | C₈₂H₁₀₆F₂N₁₀O₂₂ | 1621.8 | 97 | 7.23 |
| L3-PIII-1 | PIII-1 | DIBAC-suc-PEG4-GGF-PIII-1 | 1.96 | C₇₁H₈₇F₂N₇O₁₇ | 1348.5 | 97 | 8.57 |
| L4-PIII-1 | PIII-1 | DIBAC-suc-GGF-PIII-1 | 3.01 | C₆₀H₆₆F₂N₆O₁₂ | 1101.2 | 96 | 9.08 |
| L5-PIII-1 | PIII-1 | DIBAC-suc-SGGG-PIII-1 | −1.90 | C₅₈H₆₈F₂N₈O₁₅ | 1155.2 | >99 | 7.78 |
| L1-PIII-4 | PIII-4 | DIBAC-suc-PEG4-vcPAB-PIII-4 | 2.25 | C₇₉H₁₀₂F₂N₁₀O₂₀ | 1549.7 | 98 | 7.94 |
| L6-PII-9 | PII-9 | DIBAC-suc-PEG4-PII-9 | 3.96 | C₆₁H₇₈N₄O₁₄ | 1091.3 | 99 | 9.04 |
| L7-PII-1 | PII-1 | DIBAC-suc-PII-1 | 4.40 | C₄₇H₅₃N₃O₉ | 804.0 | 98 | 9.30, 9.49 |
| L4-PI | PI | DIBAC-GGFG-NHCH₂-Dex | 2.26 | C₅₇H₆₃FN₆O₁₁ | 1027.2 | >99 | 8.24 |
| L1-PII-11 | PII-11 | DIBAC-PEG₄-vcPAB- | 4.44 | C₇₉H₁₀₅N₉O₁₉ | 1484.8 | 98 | 8.23 |
| | | A-NMeCH₂-Bud | | | | | |
| L1-PII-12 | PII-12 | DIBAC-PEG₄-vcPAB- | 4.49 | C₈₀H₁₀₅N₉O₁₉ | 1496.8 | | 8.57 |
| | | amino-piperidinone-CH₂-Bud | | | | | |
| L8-PII-1 | PII-1 | COT-PEG₄-EvcPAB-G-NHCH₂-Bud | 2.02 | C₇₃H₁₀₇N₉O₂₂ | 1462.7 | 99 | 6.73 |
| L9-PII-1 | PII-1 | COT-GG-NHCH₂-Bud | 2.55 | C₄₀H₅₅N₃O₁₀ | 737.9 | 99 | 8.45 |
| L10-PII-1 | PII-1 | COT-GGG-NHCH₂-Bud | 1.45 | C₄₂H₅₈N₄O₁₁ | 794.9 | >99 | 7.96 |
| L11-PII-2 | PII-2 | COT-AAA-NHCH₂-Bud | 3.15 | C₄₅H₆₄N₄O₁₁ | 837.0 | >99 | 8.36, 8.40 |
| L12-PII-2 | PII-2 | COT-A^{d}AA-NHCH₂-Bud | 3.15 | C₄₅H₆₄N₄O₁₁ | 837.0 | >99 | 8.21, 8.24 |
| L13-PII-1 | PII-1 | COT-GGGG-NHCH₂-Bud | 0.34 | C₄₄H₆₁N₅O₁₂ | 852.0 | 99 | 7.86 |
| L14-PII-1 | PII-1 | DIBAC-GGFG-NHCH₂-Bud | 3.31 | C₆₀H₆₈N₆O₁₂ | 1065.2 | >99 | 8.97 |
| L15-PII-1 | PII-1 | COT-SGGGG-NHCH₂-Bud | −1.24 | C₄₇H₆₆N₆O₁₄ | 939.1 | 98 | 7.56 |
| L16-PII-1 | PII-1 | COT-KGGGG-NHCH₂-Bud | −0.53 | C₅₀H₇₃N₇O₁₃ | 980.2 | 99 | 6.57 |
| L17-PII-1 | PII-1 | COT-PEG₄-G-NHCH₂-Bud | 2.60 | C₄₉H₇₃N₃O₁₄ | 928.1 | >99 | 8.73 |
| L6-PII-13 | PII-13 | DIBAC-PEG₄-G- Azetidine-Bud | 3.88 | C₆₀H₇₆N₄O₁₄ | 1077.3 | >99 | 10.01 |
| L18-PII-1 | PII-1 | COT-EDA-(GLC)PAB-G-NHCH₂-Bud | 1.53 | C₅₅H₇₄N₄O₁₈ | 1079.2 | | 8.39 |
| L19-PIII-1 | PIII-1 | BCN-PEG₄-vcPAB-G-NHCH₂-III | 3.23 | C₆₉H₉₈F₂N₈O₁₉ | 1381.6 | >99 | 8.04 |
| L9-PIII-1 | PIII-1 | COT-GG-NHCH₂-III | 2.26 | C₄₀H₅₃F₂N₃O₁₀ | 773.9 | >99 | 9.52 |
| L12-PIII-5 | PIII-5 | COT-A^{d}AA-NHCH₂-III | 2.86 | C₄₅H₆₂F₂N₄O₁₁ | 873.0 | 97 | 8.31 |
| L13-PIII-1 | PIII-1 | COT-GGGG-NHCH₂-III | 0.05 | C₄₄H₅₉F₂N₅O₁₂ | 888.0 | >99 | 7.90 |
| L1-PIV | PIV | DIBAC-PEG₄-vcPAB-G-NHCH₂-IV | 4.33 | C₇₁H₉₇N₉O₁₅ | 1316.6 | 97 | 8.38 |
| L14-PIV | PIV | DIBAC-GGFG-NHCH₂-IV | 7.10 | C₇₁H₈₀N₈O₁₁ | 1221.4 | >99 | 8.88 |

¹All the linker-payloads were tested in HPLC using method B; for the compounds containing two chiral centers, two representative peaks were observed by LCMS.

TABLE 3c
Cleavage of Linker-N—C—O-Glucocorticoids

| Quenched | | | | | |
|---|---|---|---|---|---|
| linker- | | | HPLC Rt | | Solubility |
| payloads | Azides | Linker-payloads | (min) | m/z | (mg/mL) |
| Q1L1-PI | taurine-PEG4- | DIBAC-suc-PEG4-vcPAB- | 1.74(B) | 712.8 | >1 |
| | azide | PI | [LCMS] | [(M − Bud)/2 + H] | |
| Q2L1-PII-1 | CD-N₃ | DIBAC-suc-PEG4-vcPAB- | 6.76 (B) | 819.0 | >1 |
| | | PII-1 | | (M/3 + H) | |
| Q1L1-PII-2 | taurine-PEG4- | DIBAC-suc-PEG4-vcPAB- | 6.83 (B) | 623.8 | >1 |
| | azide | PII-2 | | (M/3 + H), | |
| | | | | 720.0 | |
| | | | | [(M − Bud)/2 + H] | |
| Q1L1-PII-3 | taurine-PEG4- | DIBAC-suc-PEG4-vcPAB- | 7.21 (B) | 757.8 | >1 |
| | azide | PII-3 | | [(M − Bud)/2 + H] | |
| Q1L1-PII-4 | taurine-PEG4- | DIBAC-suc-PEG4-vcPAB- | 6.65 (B) | 741.5 | >1 |
| | azide | PII-4 | | [(M − Bud)/2 + H] | |
| Q1L1-PII-5 | taurine-PEG4- | DIBAC-suc-PEG4-vcPAB- | 6.82 (B) | 755.5 | >1 |
| | azide | PII-5 | | [(M − Bud)/2 + H] | |
| Q1L1-PII-6 | taurine-PEG4- | DIBAC-suc-PEG4-vcPAB- | 6.99, | 726.7 | >1 |
| | azide | PII-6 | 7.05 (B) | [(M − Bud)/2 + H] | |
| Q1L1-PII-7 | taurine-PEG4- | DIBAC-suc-PEG4-vcPAB- | 8.21, | 648.0 | >1 |
| | azide | PII-7 | 8.27 (B) | (M/3 + H), | |
| | | | | 758.0 | |
| | | | | [(M − Bud)/2 + H] | |
| Q1L1-PII-8 | taurine-PEG4- | DIBAC-suc-PEG4-vcPAB- | 6.92 (B) | 935.0 | >1 |
| | azide | PII-8 | | (M/2 + H), | |
| | | | | 720.0 | |
| | | | | [(M − Bud)/2 + H] | |
| Q1L1-PII-9 | taurine-PEG4- | DIBAC-suc-PEG4-vcPAB- | 7.03 (B) | 732.8 | >1 |
| | azide | PII-9 | | [(M − Bud)/2 + H] | |
| Q1L1-PIII-4 | taurine-PEG4- | DIBAC-suc-PEG4-vcPAB- | 6.68 (B) | 741.4 | >1 |
| | azide | PIII-4 | | [(M − Bud)/2 + H] | |
| Q2L3-PIII-1 | CD-N₃ | DIBAC-suc-PEG4-GGF- | 6.88 (B) | 940.5 | >1 |
| | | PIII-1 | | [(M − Bud)/2 + H] | |
| Q1L5-PIII-1 | taurine-PEG4- | DIBAC-suc-SGGG-PIII-1 | 6.24 (B) | 1087.3 | >1 |
| | azide | | | (M − Bud) | |
| Q2L6-PII-9 | CD-N₃ | DIBAC-suc-PEG4-PII-9 | 6.94 (B) | 1067.6 | >1 |
| | | | | (M/2 + Na), | |
| Q2L7-PII-1 | CD-N₃ | DIBAC-suc-PII-1 | 6.74 (B) | 901.7 | >1 |
| | | | | (M/2 + H) | |

TABLE 3d
Structures of Quenched Linker-N—C—O-Glucocorticoids

| QLP# | Structures |
|---|---|
| Q1L1- PI | 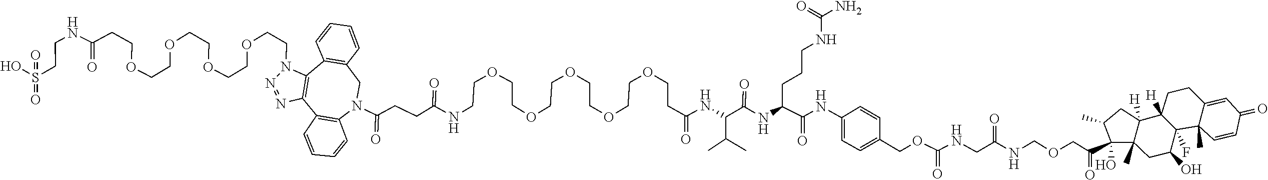 |
| Q2L1- PII-1 | 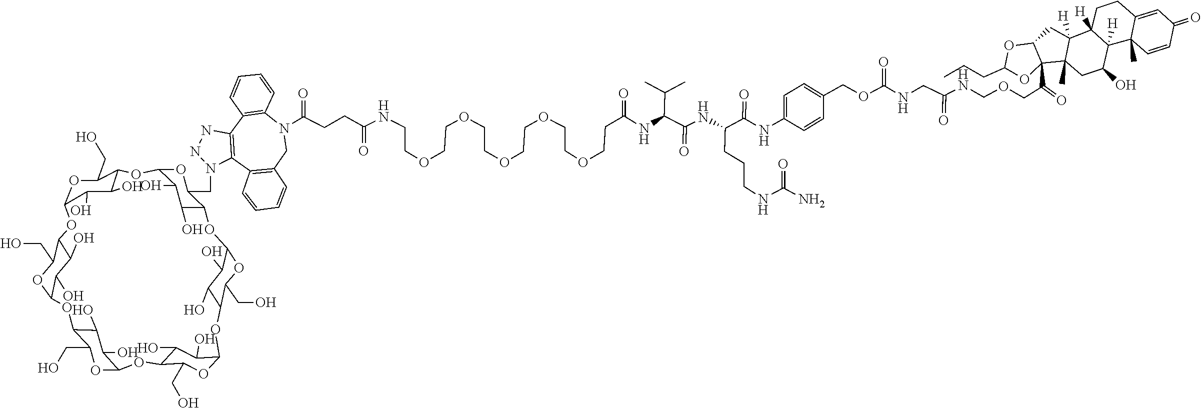 |
| Q1L1- PII-2 | 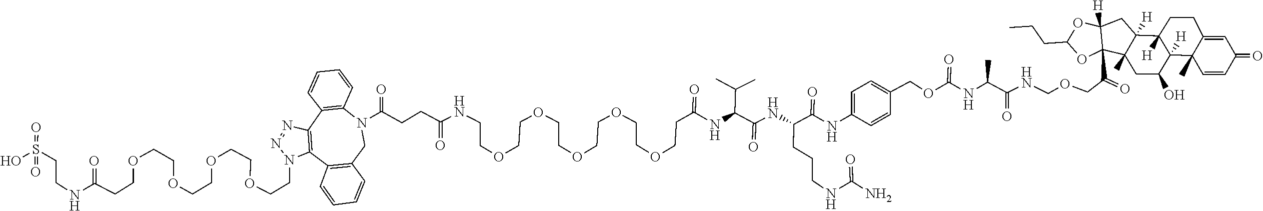 |
| Q1L1- PII-3 | 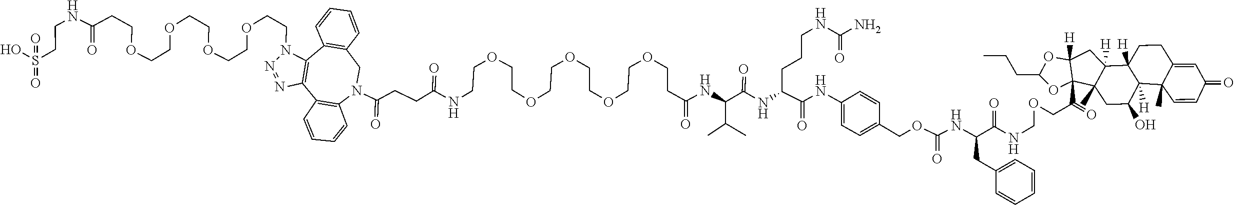 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)[C@@H](Cc2ccccc2)NC(=O)OCc2ccc(NC(=O)[C@@H](CCCNC(N)=O)NC(=O)[C@H](NC(=O)CCOCCOCCOCCOCCOCCNC(=O)CCC(=O)N3Cc4ccccc4-c4c(nnn4CCOCCOCCOCCOCCC(=O)NCCS(=O)(=O)O)-c4ccccc43)C(C)C)cc2)O1] |
| Q1L1- PII-4 | 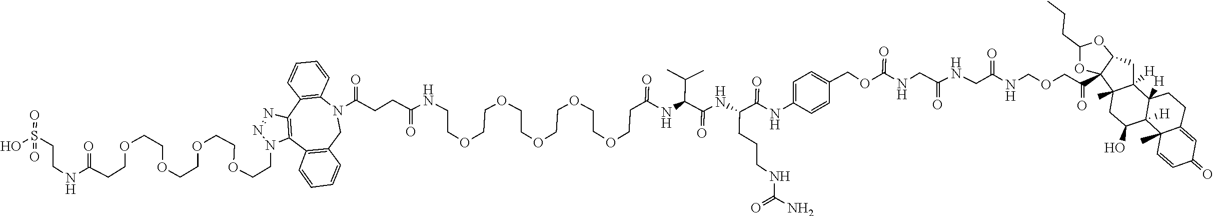 |
| Q1L1- PII-5 | 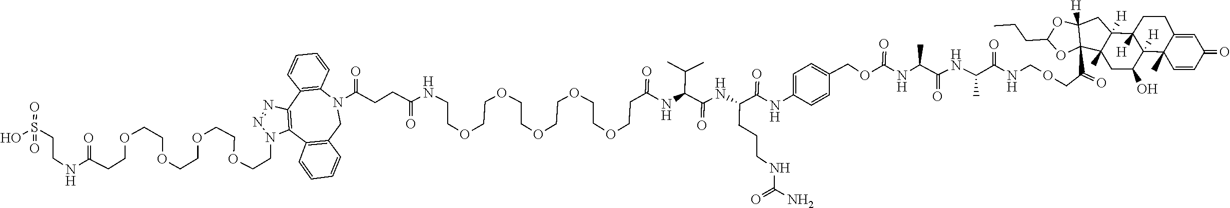 |
| Q1L1- PII-6 | 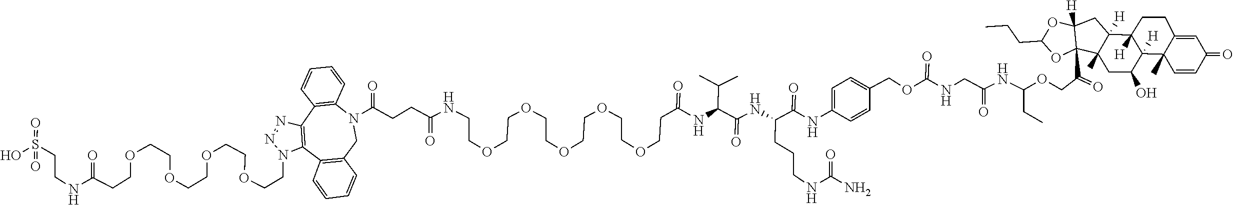 |
| Q1L1- PII-7 | 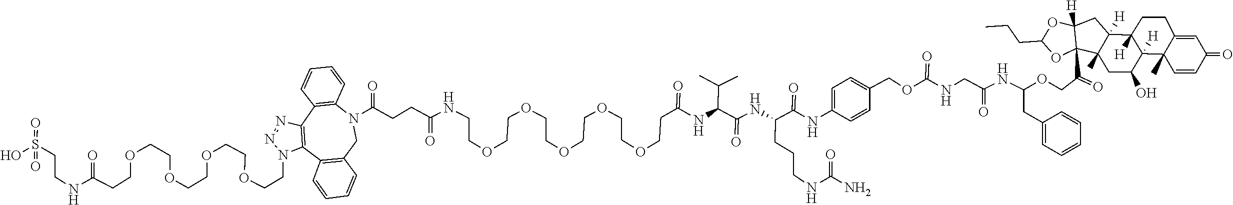 |
| Q1L1- PII-8 | 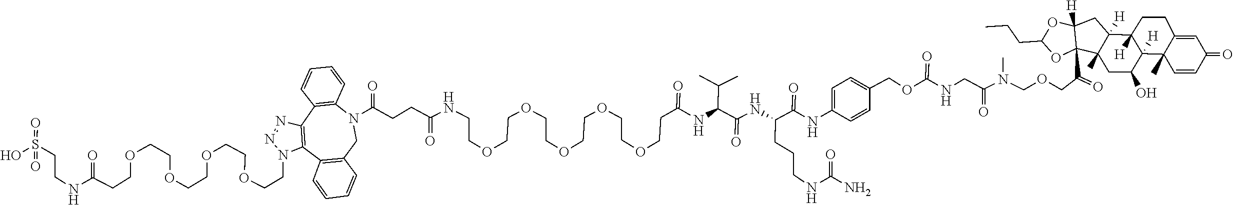 |
| Q1L1- PII-9 | 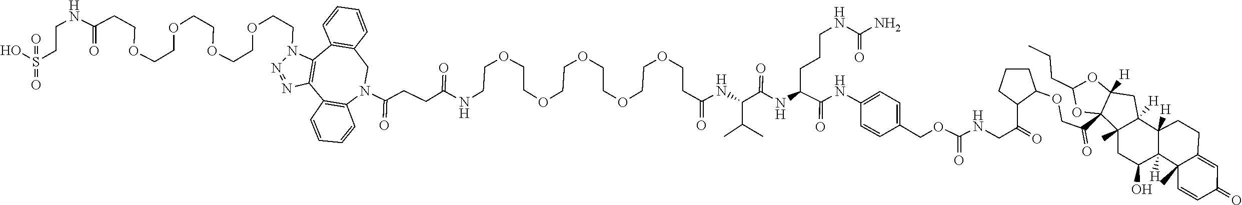 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCC2C(=O)CNC(=O)OCc2ccc(NC(=O)[C@H](CCCNC(N)=O)NC(=O)[C@@H](NC(=O)CCOCCOCCOCCOCCOCCNC(=O)CCC(=O)N3Cc4ccccc4-c4c(nnn4CCOCCOCCOCCOCCC(=O)NCCS(=O)(=O)O)-c4ccccc43)C(C)C)cc2)O1] |
| Q2L3- PIII-1 | 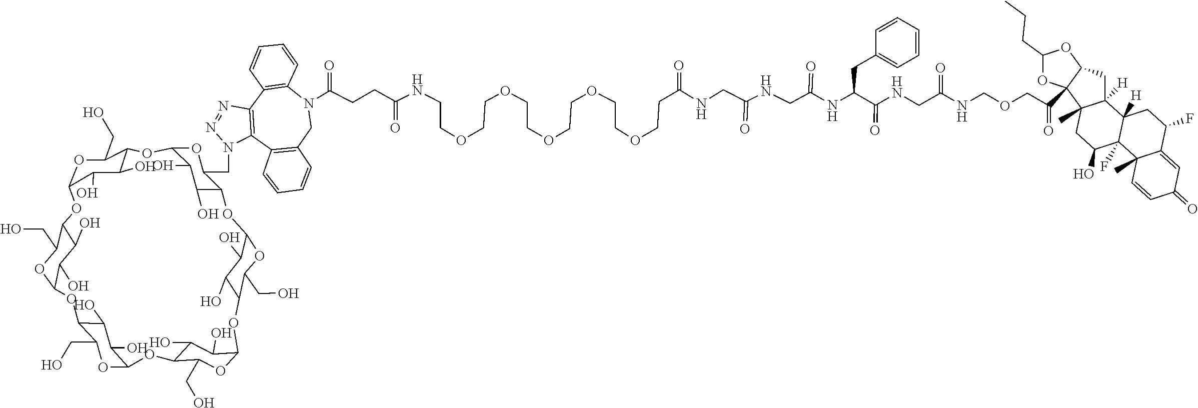 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)[C@H](Cc2ccccc2)NC(=O)CNC(=O)CNC(=O)CCOCCOCCOCCOCCOCCNC(=O)CCC(=O)N2Cc3ccccc3-c3c(nnn3CC3OC4OC5C(CO)OC(OC6C(CO)OC(OC7C(CO)OC(OC8C(CO)OC(OC9C(CO)OC(OC3C(O)C4O)C(O)C9O)C(O)C8O)C(O)C7O)C(O)C6O)C(O)C5O)-c3ccccc32)O1] |
| Q1L5- PIII-1 | 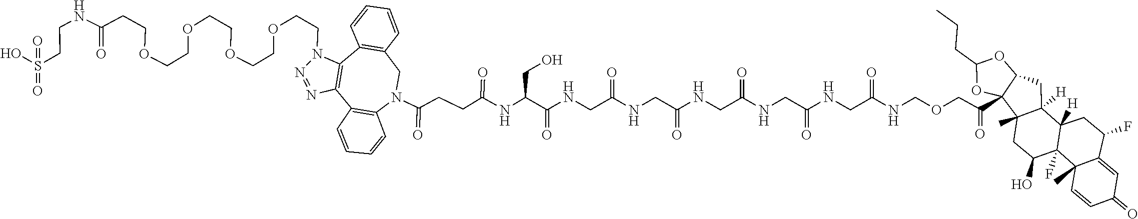 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)CNC(=O)CNC(=O)CNC(=O)CNC(=O)[C@H](CO)NC(=O)CCC(=O)N2Cc3ccccc3-c3c(nnn3CCOCCOCCOCCOCCC(=O)NCCS(=O)(=O)O)-c3ccccc32)O1] |
| Q1L1- PIII-4 | 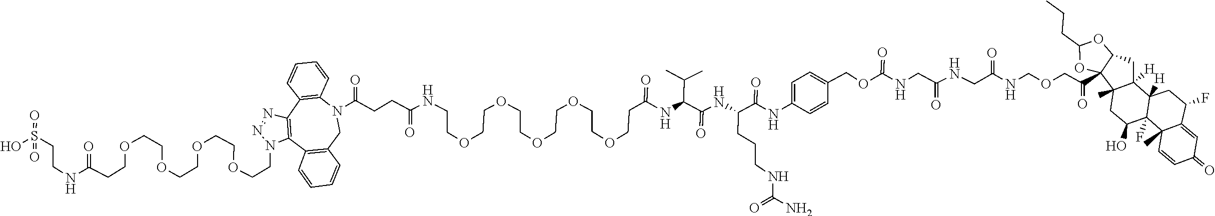 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)CNC(=O)OCc2ccc(NC(=O)[C@H](CCCNC(N)=O)NC(=O)[C@@H](NC(=O)CCOCCOCCOCCOCCOCCNC(=O)CCC(=O)N3Cc4ccccc4-c4c(nnn4CCOCCOCCOCCOCCC(=O)NCCS(=O)(=O)O)-c4ccccc43)C(C)C)cc2)O1] |
| Q2L6- PII-9 | 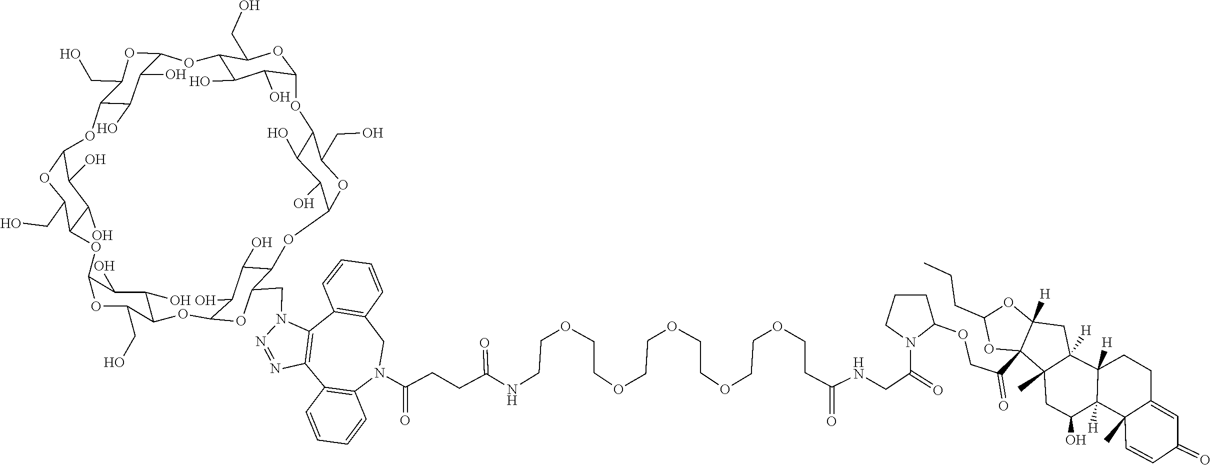 |
| Q2L7- PII-1 | 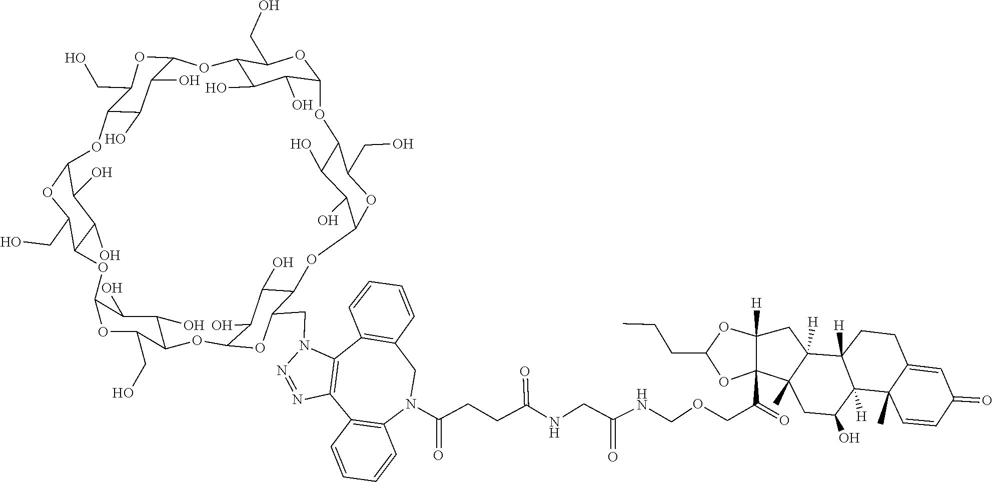 |

| | |
|---|---|
| ADC | Antibody-drug conjugate |
| Aglycosylated antibody | Antibody that does not have any glycan |
| aq. | Aqueous |
| Boc | N-tert-butoxycarbonyl |
| BupH | Thermo Scientific Prod# 28372, containing 100 mM sodium |
| | phosphate and 150 mM sodium chloride, potassium free, pH was |
| | adjusted from 7.2 to 7.6-7.8 MQ, unless otherwise noted. |
| COT | Cyclooctynol |
| CD | cyclodextrin |
| Da | Dalton |
| DAR | Drug to antibody ratio |
| DCM | Dichloromethane |
| DIBAC | Dibenz[b,f]azocine, 11,12-didehydro-5,6-dihydro- |
| DIBAC-Suc | Dibenz[b,f]azocine-5(6H)-butanoic acid, 11,12-didehydro |
| DIBACT | 3H-Benzo[c]-1,2,3-triazolo[4,5-e][1]benzazocine, 8,9-dihydro- |
| DIPEA | Diisopropylethylamine |
| DMF | N,N-dimethylformamide |
| DMSO | Dimethylsulfoxide |
| EC | Enzyme commission |
| ELSD | Evaporating light scattering detector |
| Equiv. | Equivalent |
| ESI | Electrospray ionization |
| g | Gram |
| HATU | 2-(7-Aza-1H-benzotriazole-1-yl)-1,1,3,3-tetramethyluronium |
| | hexafluorophosphate |
| HC | Heavy chain of immunoglobulin |
| HEK | Human embryonic kidney (cells) |
| HPLC | High performance liquid chromatography |
| h, hr, or hrs | Hours |
| LC | Light chain of immunoglobulin |
| LCh | Liquid chromatography |
| MALDI | Matrix-assisted laser desorption/ionization |
| mg | milligrams |
| min | minutes |
| mL | milliliters |
| mmh | myc-myc-hexahistidine tag |
| μL | microliters |
| mM | millimolar |
| μM | micromolar |
| MS | Mass spectrometry |
| MSD | Mass-selective detector |
| MW | Molecular weight |
| NHS | N-hydroxy succinimide |
| nM | nanomolar |
| NMR | Nuclear magnetic resonance |
| PAB | Para-aminobenzyloxy(carbonyl) |
| PBS | 10 mM sodium phosphate buffer and 150 mM sodium chloride |
| PBSg | 10 mM phosphate, 150 mM sodium chloride, 5% glycerol |
| PEG | Polyethyleneglycol |
| ppm | Parts per million (chemical shift) |
| PPTS | pyridinium p-toluenesulfonate |
| RP | Reversed phase |
| RT or rt | Room temperature |
| Sat. | Saturated |
| SDS-PAGE | Sodium dodecylsulfate polyacrylamide gel electrophoresis |
| SEC | Size exclusion chromatography |
| Suc | Succinic acid |
| TCEP | Tris(2-carboxyethyl)phosphine hydrochloride |
| TEA | Triethylamine |
| TFA | Trifluoroacetic acid |
| TG | Transglutaminase |
| THF | Tetrahydrofuran |
| TOF | Time-of-flight |
| UPLC | Ultra Performance Liquid Chromatography |
| UV | Ultraviolet |
| VC | Valine-citrulline |

### Synthesis of Drug/Payload D*-OH

Dexamethasone I and Budesonide II are commercially available steroidal drugs (D*-OH). 6,11-2F-budesonide III is reported in WO 2018/213077 A1. LXR Agonist IV was synthesized from LXR Agonist 7 (LXR Agonist 7 is reported in WO 2018/213077 A1 and WO 2018/213082 A1), as shown below.

### (1S,4aS,10aR)-N-[(1S,4aS,10aR)-6-(2-Hydroxyacetamido)-1,4a-dimethyl-1,2,3,4,4a,9,10,10a-octahydrophenanthrene-1-carbonyl]-6-hydroxy-1,4a-dimethyl-1,2,3,4,4a,9,10,10a-octahydrophenanthrene-1-carboxamide (IV)

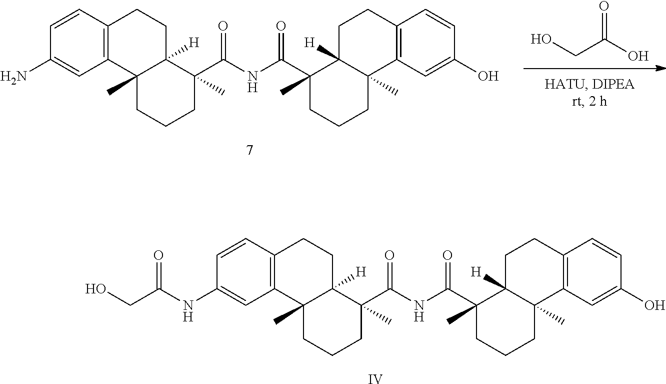

To a solution of 7 (10 mg, 19 μmol, see WO 2018213082 A1) in DMF (2 mL) were added HATU (14 mg, 38 μmol) and DIPEA (9.8 mg, 76 μmol) at RT. The mixture was stirred at RT for 15 minutes before the addition of Glycolic acid (1.7 mg, 23 μmol). The reaction mixture was stirred at RT for 2 hours, which was monitored by LCMS. The resulting mixture was directly purified by prep-HPLC to give compound IV (5.7 mg, 51% yield) as a white solid. ESI m/z: 587 (M+1)⁺. ¹H NMR (400 MHz, DMSO_{d6}) δ 9.46 (s, 1H), 9.00 (s, 1H), 8.11 (s, 1H), 7.59 (d, J=1.8 Hz, 1H), 7.49 (dd, J=8.3, 1.8 Hz, 1H), 6.96 (d, J=8.3 Hz, 1H), 6.82 (d, J=8.3 Hz, 1H), 6.63 (d, J=2.3 Hz, 1H), 6.50 (dd, J=8.3, 2.3 Hz, 1H), 5.64 (t, J=5.9 Hz, 1H), 3.94 (d, J=5.9 Hz, 2H), 2.93-2.66 (m, 4H), 2.37-2.06 (m, 6H), 2.03-1.75 (m, 4H), 1.68-1.50 (m, 4H), 1.28 (s, 3H), 1.27 (s, 3H), 1.36-1.20 (m, 2H), 1.20-1.08 (m, 2H), 1.00 (s, 3H), 0.99 (s, 3H) ppm.

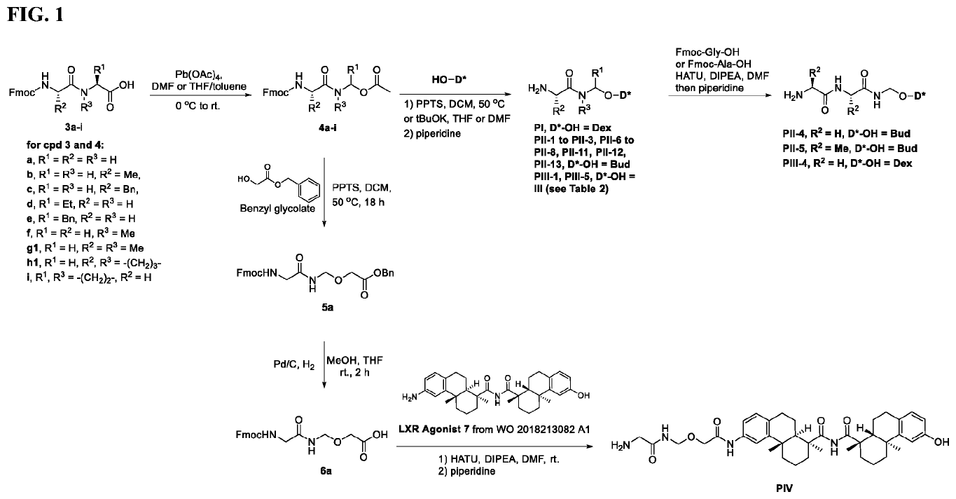

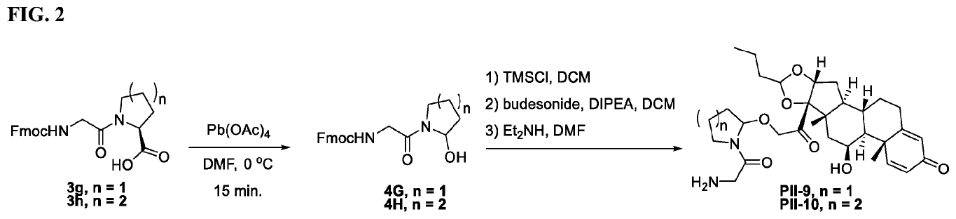

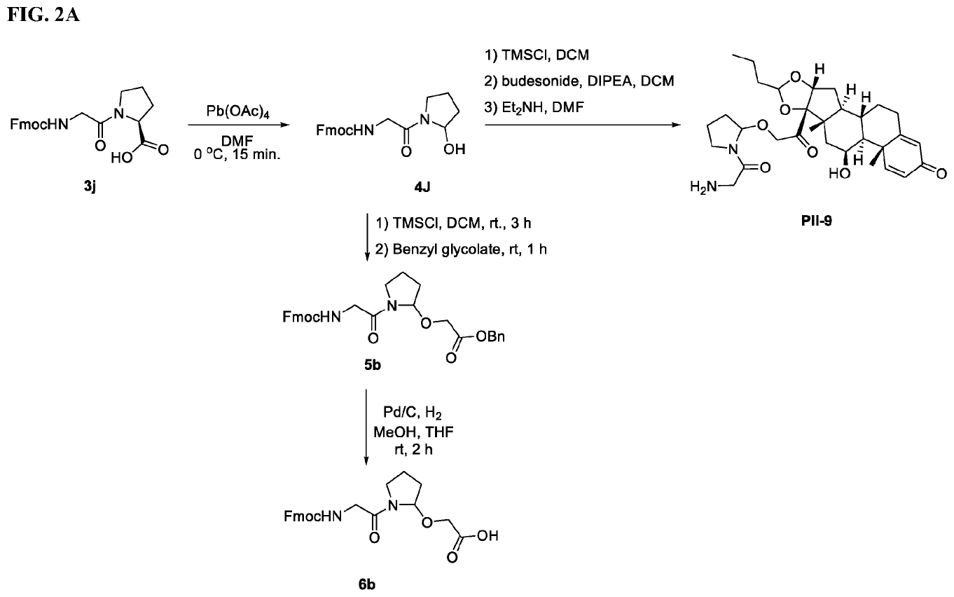

Most payloads were synthesized according to FIG. 1, except payloads PII-9 FIG. 2 and FIG. 2A; and PII-10 FIG. 2. Intermediates 5a and 6a, were synthesized as described in WO 2015/155998 A1.

### FIG. 1 Starting Dipeptides 3a-h

### 3a: Fmoc-Gly-Gly-OH, CAS: 35665-38-4

### 3b: Fmoc-Ala-Gly-OH, CAS: 116747-54-7

### 3e: Fmoc-Phe-Gly-OH, CAS: 169624-67-3

### 3d: Fmoc-Gly-Abu-OH, CAS: 2171191-91-4

### 3e: Fmoc-Gly-Phe-OH, CAS: 117370-45-3

### 3f: Fmoc-Gly-Sar-OH, CAS: 1499188-24-7

### 3g and 3j: Fmoc-Gly-Pro-OH, CAS: 212651-48-4

### 3g1: Fmoc-Ala-Sar-OH, CAS: 2171221-36-4

3h1: Fmoc-(R,S)-3-amino-1-carboxymethyl-valerolactame, CAS: 209163-25-7
3i: Fmoc-Gly-2-azetidinecarboxylic acid, 2171729-15-8

### 1-(2-{[(9H-Fluoren-9-ylmethoxy)carbonyl]amino}acetyl)piperidine-2-carboxylic acid (3h, Fmoc-Gly-Pip-OH)

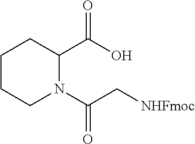

To a mixture of Pipecolinic acid (0.59 g, 4.6 mmol) and Fmoc-Gly-OSu (1.80 g, 4.6 mmol) in DMF (10 mL) was added DIPEA (1.8 g, 14 mmol), and the mixture was stirred at RT for 2 hours, which was monitored by LCMS. The resulting mixture was directly purified by prep-HPLC to give compound 3h (1.0 g, 53% yield) as a white solid. ESI m/z: 409 (M+1)⁺. ¹H NMR (400 MHz, DMSO_{d6}) δ 7.89 (d, J=7.6 Hz, 2H), 7.73 (d, J=7.2 Hz, 2H), 7.42 (t, J=7.2 Hz, 2H), 7.35-7.29 (m, 3H), 4.32-4.21 (m, 4H), 4.03-3.95 (m, 1H), 3.67-3.61 (m, 1H), 2.78-2.73 (m, 2H), 2.23-2.14 (m, 1H), 1.61-1.59 (m, 2H), 1.50-1.25 (m, 4H) ppm.

### Synthesis of Intermediates 4

### General Procedure A for Intermediates 4

To a mixture of peptide 3 (1.0 equiv.) in THE (30 mL per gram of 3) and toluene (10 mL per gram of 3) were added pyridine (1.2 equiv.) and lead tetraacetate (1.2 equiv.). The resulting mixture was stirred for 5 hours at 80° C., and monitored by LCMS. After cooling to RT, the mixture was filtered through Celite, and the filtrate was concentrated in vacuo or directly diluted with ethyl acetate. The organic solution was washed with water and brine, dried over anhydrous sodium sulfate, and concentrated in vacuo. The residue was purified by silica gel column chromatography (0-10% ethyl acetate in petroleum ether) to give compound 4 (67-67% yield) as a white solid.

### General Procedure B for Intermediates 4

To a mixture of peptide 3 (1.0 equiv.) in dry DMF (2 mL per gram of 3) was added lead tetraacetate (1.2 equiv.). The resulting mixture was stirred at RT for 5-30 minutes, and monitored by LCMS. The resulting mixture was filtered through Celite, and the filtrate was diluted with ethyl acetate, washed with water and brine, dried over anhydrous sodium sulfate, and concentrated in vacuo. The residue was purified by silica gel column chromatography (0-10% ethyl acetate in petroleum ether) to give compound 4 (48-78% yield) as a white solid.

TABLE 3e
Results of Intermediates 4 With Different R¹ Substituents

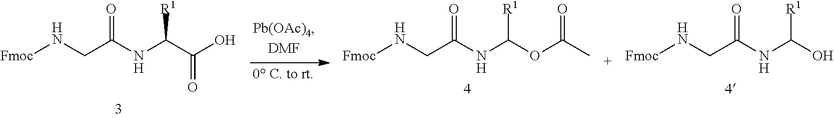

| | | | | Content in LCMS (%) | | |
|---|---|---|---|---|---|---|
| | | Separated | Reaction | Starting | | Desired |
| Entry | R¹ | yield (%) | time (h) | material 3 | Side-product 4′ | product 4 |
| 1 | H | 40 | 1 | 70 | 10 | 20 |
| | | | 5 | 30 | 20 | 50 |
| 2 | Me | 0 | 1 | 90 | 10 | Trace |
| | | | 5 | 0 | >95 | Trace |
| 3 | Et | Crude* | 1 | 10 | 40 | 50 |
| | | | 5 | 0 | 85 | Trace |
| 4 | Bn | 48 | 1 | 80 | 0 | 20 |
| | | | 5 | 10 | 10 | 80 |
| 5 | Ph | 0 | 1 | 0 | 75 | 0 |

* The acetate was easily hydrolyzed during the purification.

TABLE 4
Summary of the Synthesis of Intermediates 4

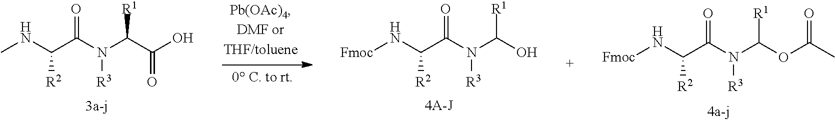

| | | | | | | Contents of reaction mixture in LCMS (%) | | |
|---|---|---|---|---|---|---|---|---|
| | | | | Separated | Reaction | Starting | Alcohol | Acetate |
| Entry | R² | R³ | R¹ | yield (%) | time (h) | material 3 | 4A-J | 4a-j |
| a/A | H | H | H | 40 | 1 | 70 | 10 | 20 |
| | | | | | 5 | 30 | 20 | 50 |
| | | | | 67 | 5 | 0 | 20 | 70 |
| b/B | Me | H | H | 78 | 1 | 0 | 0 | 74 |
| c/C | Bn | H | H | 30 | 0.5 | 0 | 0 | 80 |
| d/D | H | H | Et | Crude¹ | 1 | 10 | 40 | 50 |
| | | | | | 5 | 0 | 85 | Trace |
| e/E | H | H | Bn | 48 | 1 | 80 | 0 | 20 |
| | | | | | 5 | 10 | 10 | 80 |
| f/F | H | Me | H | 19 | 1 | 0 | 20 | 70 |
| | | | | (crude)¹ | | | | |
| g/G | Me | Me | H | 53 | 3 | 0 | 0 | 66 |

| | | | | | | | |
|---|---|---|---|---|---|---|---|
| h/H | 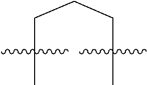 [SMILES: *CCC*] | H | 15 | 2 | 5 | 25 | 47 |

| | | | | | | | |
|---|---|---|---|---|---|---|---|
| i/I | H | 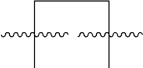 [SMILES: *CC*] | 54 | 1 | 0 | 0 | 69 |
| j/J | H | 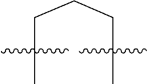 [SMILES: *CCC*] | 53² | 4 | 0 | 74 | 0 |

1. The acetate was easily hydrolyzed and was contaminated with alcohol.
2. The yield of alcohol 4J.

### (2-(((9H-Fluoren-9-yl)methoxy)carbonylamino)acetamido)methyl acetate (4a) (CAS: 1599440-06-8)

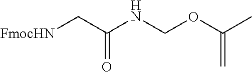

Following General Procedure A for intermediate 4, or the synthesis reported in Tetrahedron 74 (2018) 1951-1956, compound 4a (3.0 g, 67% yield) was obtained as a white solid. ESI m/z: 391 (M+²³)⁺.

### [(2S)-2-{[(9H-Fluoren-9-ylmethoxy)carbonyl]amino}propanamido]methyl acetate (4b)

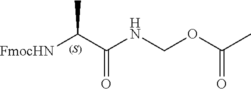

Following General Procedure B for intermediate 4, compound 4b (30 mg, 78% yield) was obtained as a white solid. ESI m/z: 405 (M+23)⁺.

### [(2S)-2-{[(9H-Fluoren-9-ylmethoxy)carbonyl]amino}-3-phenylpropanamido]methyl acetate (4c)

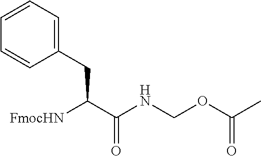

Following General Procedure B for intermediate 4, compound 4c (0.31 g, 30% yield) was obtained as a white solid. ESI m/z: 481 (M+23)⁺. ¹H NMR (400 MHz, DMSO_{d6}) δ 9.14 (t, J=6.8 Hz, 1H), 7.88 (d, J=7.6 Hz, 2H), 7.73-7.70 (m, 1H), 7.64 (t, J=8.8 Hz, 2H), 7.43-7.39 (m, 2H), 7.34-7.24 (m, 6H), 7.22-7.17 (m, 1H), 5.12 (dd, J=7.2, 2.8 Hz, 2H), 4.31-4.26 (m, 1H), 4.19-4.11 (m, 3H), 3.01-2.94 (m, 1H), 2.84-2.78 (m, 1H), 2.00 (s, 3H) ppm.

### 1-(2-{[(9H-Fluoren-9-ylmethoxy)carbonyl]amino}acetamido)propyl acetate (4d)

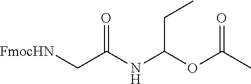

Following General Procedure B for intermediate 4, crude compound 4d (0.35 g) was obtained, and was used in the next step without purification. ESI m/z: 419 (M+23)⁺.

### 1-(2-{[(9H-Fluoren-9-ylmethoxy)carbonyl]amino}acetamido)-2-phenylethyl acetate (4e)

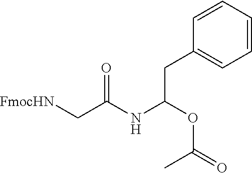

Following General Procedure B for intermediate 4, compound 4e (0.30 g, 48% yield) was obtained as a white solid. ESI m/z: 481 (M+23)⁺.

### (2-{[(9H-Fluoren-9-ylmethoxy)carbonyl]amino}-N-methylacetamido)methyl acetate (4f)

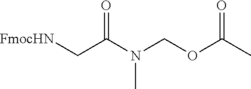

Following General Procedure B for intermediate 4, crude compound 4f (0.40 g, 19% yield) was obtained as a white solid, which was contaminated with the hydrolysis product alcohol after purification. The mixture was used in the next step without further purification. ESI m/z: 405 (M+23)⁺.

### [(2S)-2-{[(9H-Fluoren-9-ylmethoxy)carbonyl]amino}-N-methylpropanamido]methyl acetate (4g)

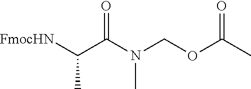

Following the General Procedure B for intermediate 4, crude compound 4g (0.44 g, 53% yield) was obtained as a white solid after purification by prep-HPLC. ESI m/z: 419 (M+23)⁺.

### (3-{[(9H-Fluoren-9-ylmethoxy)carbonyl]amino}-2-oxopiperidin-1-yl)methyl acetate (4h)

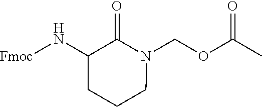

Following the General Procedure B for intermediate 4, crude compound 4h (0.10 g, 15% yield) was obtained as a white solid. ESI m/z: 431 (M+23)⁺.

### 1-(2-{[(9H-Fluoren-9-ylmethoxy)carbonyl]amino}acetyl)azetidin-2-yl acetate (4i)

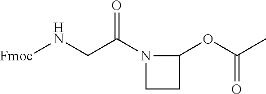

Following the General Procedure B for intermediate 4, crude compound 4i (0.18 g, 47% yield) was obtained as a white solid. ESI m/z: 395 (M+1)⁺.

### 9H-Fluoren-9-ylmethyl N-[2-(2-hydroxypyrrolidin-1-yl)-2-oxoethyl]carbamate (4J)

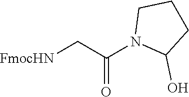

Following the general procedure B for intermediate 4, crude compound 4J (50 mg, 53% yield) was obtained as a white solid. No acetate intermediate was obtained. ESI m/z: 389 (M+23)⁺. ¹H NMR (400 MHz, DMSO_{d6}) δ 7.90 (d, J=7.4 Hz, 2H), 7.73 (d, J=7.5 Hz, 2H), 7.47-7.37 (m, 3H), 7.33 (t, J=7.3 Hz, 2H), 5.86 (br s, 1H), 5.48 (d, J=4.0 Hz, 0.25H), 5.39 (d, J=4.0 Hz, 0.75H), 4.33-4.18 (m, 3H), 3.96 (d, J=6.0 Hz, 1.5H), 3.75 (d, J=6.0 Hz, 0.5H), 3.59-3.33 (m, 1H), 3.22-3.11 (m, 1H), 2.00-1.59 (m, 4H) ppm.

### Synthesis of Intermediate 5a and Payloads PI, PII-1, PII-2, PII-3, PII-6, PII-7, PII-8, PII-11, PII-12, PII-13, and PIII-1 (FIG. 1)

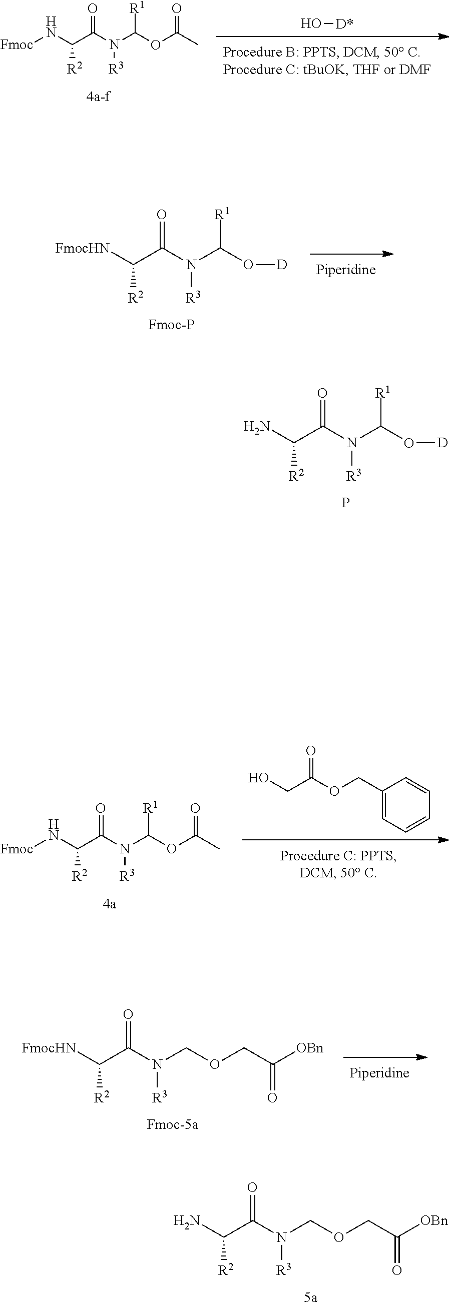

### General Procedure A for Payloads

A mixture of compound 4 (1 equiv.), corresponding alcohol (HO-D* or benzyl glycolate) (1 equiv.) and PPTS (0.1 equiv.) in DCM (40 mL per gram of alcohol) was added into a 10 mL-sealed tube. The mixture was sealed and stirred at 50° C. overnight, and monitored by LCMS. The resulting mixture was concentrated in vacuo and the residue was directly purified by prep-HPLC to give Fmoc-P or intermediate 5a. Fmoc-P was dissolved in DMF (40 mM). To the solution was added piperidine (4 equiv.), and the mixture was stirred at RT for an hour until Fmoc was totally removed, as monitored by LCMS. The reaction mixture was directly purified by prep-HPLC to give payload P (4.1-28% yield) as a white solid.

### General Procedure B for Payloads

To a solution of compound 4 (1.0 equiv.) and corresponding alcohol (HO-D*) (1.0 equiv.) in THF (0.25 M) was added potassium tert-butoxide (2.0 equiv.) at 0° C. The mixture was stirred at RT for 3 hours until the concentration of Fmoc-P remained static, as monitored by LCMS (neither compound 4 nor alcohol was completely consumed). The reaction mixture was diluted with ethyl acetate and carefully quenched with water at 0° C. The aq. layer was extracted with ethyl acetate and chloroform. The organic layers were combined, dried over anhydrous sodium sulfate, and concentrated in vacuo. The residue was purified by silica gel column chromatography (25-70% ethyl acetate in petroleum ether) to give Fmoc-P (containing payload P), which was dissolved in DMF (40 mM). To the solution was added piperidine (4 equiv.), and the mixture was stirred at RT for 2 hours until Fmoc was totally removed, as monitored by LCMS. The reaction mixture was directly purified by prep-HPLC to give payload P (6.7-28% yield) as a white solid.

TABLE 5
Conditions For Generic Synthesis of Payloads

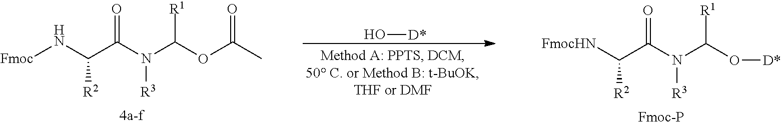

| Entry | Product Fmoc-P | R² | R³ | R¹ | Cpd 4 | HO—D* | Method | Content in LCMS (%) |
|---|---|---|---|---|---|---|---|---|
| 1 | Fmoc-PI | H | H | H | 4a | I (Dex) | A | 27 |
| 2 | Fmoc-PII-1 | H | H | H | 4a | II (Bud) | A | 40 |
| | | | | | | | B | 23 |
| 3 | Fmoc-PII-2 | Me | H | H | 4b | II (Bud) | A | No reaction |
| | | | | | | | B | 11 |
| 4 | Fmoc-PII-3 | Bn | H | H | 4c | II (Bud) | B | 42 |
| 5 | Fmoc-PII-6 | H | H | Et | 4d (crude) | II (Bud) | A | 25 |
| 6 | Fmoc-PII-7 | H | H | Bn | 4e | II (Bud) | A | No reaction |
| | | | | | | | B | 30 |
| 7 | Fmoc-PII-8 | H | Me | H | 4f | II (Bud) | A | 15 |
| 8 | Fmoc-PIII-1 | H | H | H | 4a | III | A | 42 |
| | | | | | | | B | 21 |

### 2-Amino-N-({2-[(1R,2S,10S,11S,13R,14R,15S,17S)-1-fluoro-14,17-dihydroxy-2,13,15-trimethyl-5-oxotetracyclo[8.7.0.0^{2,7}.0^{11,15}]heptadeca-3,6-dien-14-yl]-2-oxoethoxy}methyl)acetamide, TFA salt (PI)

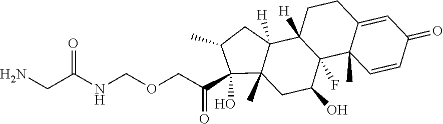 [SMILES: C[C@@H]1C[C@H]2[C@@H]3CCC4=CC(=O)C=C[C@]4(C)[C@@]3(F)[C@@H](O)C[C@]2(C)[C@@]1(O)C(=O)COCNC(=O)CN]

Following General Procedure A for payloads, starting from compound 4a and dexamethasone, payload PI (0.26 g, 18% yield) as a white solid was obtained (TFA salt). ESI m/z: 501 (M+23)⁺. ¹H NMR (400 MHz, DMSO_{d6}) δ 9.14 (t, J=6.5 Hz, 1H), 8.02 (s, 3H), 7.30 (d, J=10.2 Hz, 1H), 6.23 (dd, J=10.2, 2.0 Hz, 1H), 6.02 (s, 1H), 5.29 (d, J=2.0 Hz, 1H), 5.00 (s, 1H), 4.68-4.52 (m, 3H), 4.17 (d, J=18.4 Hz, 1H), 4.17-4.11 (m, 1H), 3.60 (s, 2H), 2.99-2.83 (m, 1H), 2.70-2.56 (m, 1H), 2.44-2.27 (m, 2H), 2.17-2.03 (m, 2H), 1.83-1.71 (m, 1H), 1.62 (q, J=11.1 Hz, 1H), 1.49 (s, 3H), 1.47-1.29 (m, 2H), 1.15-0.99 (m, 1H), 0.87 (s, 3H), 0.79 (d, J=7.2 Hz, 3H) ppm.

### 2-Amino-N-({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)acetamide (PII-1)

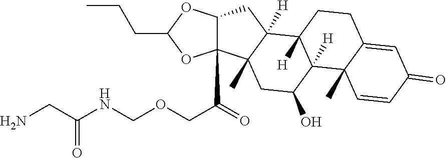

Following General Procedure B for payloads, starting from compound 4a and budesonide, payload PII-1 (50 mg, 28% yield) as a white solid was obtained. ESI m/z: 517 (M+1)⁺. ¹H NMR (400 MHz, CDCl₃) δ 8.08-8.04 (m, 1H), 7.30 (d, J=10.4 Hz, 1H), 6.25 (d, J=10.4 Hz, 1H), 6.02 (s, 1H), 5.20-5.18 (m, 1H), 4.95-4.60 (m, 3H), 4.60-4.25 (m, 3H), 3.42 (s, 2H), 3.25 (br s, 2H), 2.57-2.54 (m, 1H), 2.35-2.32 (m, 1H), 2.22-2.00 (m, 2H), 2.00-1.50 (m, 7H), 1.46 (s, 3H), 1.46-1.25 (m, 2H), 1.15-1.00 (m, 2H), 1.00-0.80 (m, 6H) ppm.

### (2S)-2-Amino-N-({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)propanamide TFA salt (PII-2)

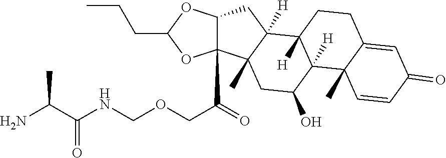

Following General Procedure B for payloads, starting from compound 4b and budesonide, payload PII-2 (6.0 mg, 6.7% yield) as a white solid was obtained (TFA salt). ESI m/z: 531 (M+1)⁺. ¹H NMR (400 MHz, DMSO_{d6}) δ 9.12-8.92 (m, 1H), 7.33 (d, J=10.1 Hz, 1H), 6.59 (br s, 2H), 6.17 (dd, J=10.1 and 1.6 Hz, 1H)), 5.92 (s, 1H), 5.17 (t, J=4.7 Hz, 0.3H), 5.03 (d, J=7.2 Hz, 0.3H), 4.78 (d, J=3.2 Hz, 1H), 4.72 (d, J=4.2 Hz, 0.7H), 4.70-4.54 (m, 3H), 4.49 (d, J=18.6 Hz, 0.7H), 4.30 (s, 1H), 4.23 (d, J=18.8 Hz, 0.7H), 4.17 (d, J=18.5 Hz, 0.3H), 3.68-3.57 (m, 1H), 3.01-3.00 (m, 1H), 2.36-2.25 (m 1H), 2.15-1.92 (m, 2H), 1.83-1.68 (m, 2H), 1.68-1.41 (m, 6H), 1.38 (s, 3H), 1.37-1.30 (m, 1H), 1.27 (d, J=7.0 Hz, 3H), 1.15-0.91 (m, 2H), 0.86 (t, J=7.4 Hz, 3H), 0.81 (s, 3H) ppm.

### (2S)-2-Amino-N-({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)-3-phenylpropanamide TFA salt (PII-3)

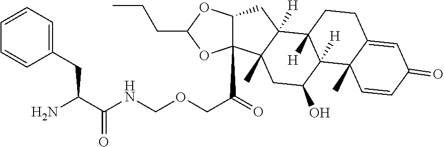

Following General Procedure B for payloads, starting from compound 4c and budesonide, payload PII-3 (84 mg, 21% yield) as a white solid was obtained (TFA salt). ESI m/z: 607 (M+1)⁺. ¹H NMR (400 MHz, DMSO_{d6}) δ 8.73 (t, J=6.2 Hz, 1H), 7.40-7.13 (m, 6H), 6.17 (d, J=10.1 Hz, 1H), 5.92 (s, 1H), 5.17 (t, J=4.7 Hz, 0.4H), 5.03 (d, J=7.1 Hz, 0.4H), 4.77 (s, 1H), 4.73 (d, J=4.1 Hz, 0.6H), 4.68-4.53 (m, 2.8H), 4.49 (d, J=2.4 Hz, 0.4H), 4.44 (s, 0.4H), 4.31 (br s, 1H), 4.18 (d, J=8.4 Hz, 0.6H), 4.13 (d, J=8.1 Hz, 0.4H), 3.52-3.36 (m, 1H), 3.02-2.85 (m, 1H), 2.70-2.55 (m, 1H), 2.36-2.22 (m, 1H), 2.16-1.92 (m, 3H), 1.88-1.64 (m, 4H), 1.63-1.48 (m, 4H), 1.48-1.40 (m, 1H), 1.381 (s, 1.8H), 1.376 (s, 1.2H), 1.36-1.23 (m, 2H), 1.18-0.91 (m, 2H), 0.90-0.75 (m, 6H) ppm.

### 2-Amino-N-{[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}acetamide (PII-4)

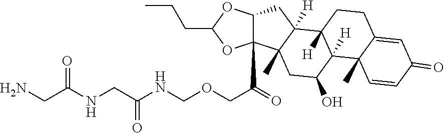

To a solution of compound PII-1 (52 mg, 0.10 mmol) in DMF (2.0 mL) were added DIPEA (40 mg, 0.31 mmol), Fmoc-Glycine (30 mg, 0.10 mmol) and HATU (50 mg, 0.13 mmol), respectively, at RT. The reaction mixture was stirred at RT for 2 hours, and monitored by LCMS. To the solution was then added piperidine (43 mg, 0.50 mmol), and the mixture was stirred at RT for an hour until Fmoc was removed, which was monitored by LCMS. The mixture was directly purified by reversed phase flash chromatography (0-100% acetonitrile in aq. ammonium bicarbonate (0.08%) to give compound PII-4 (50 mg, 91% yield) as a white solid. ESI m/z: 574 (M+1)⁺. ¹H NMR (500 MHz, DMSO_{d6}) δ 8.71 (t, J=6.7 Hz, 0.5H), 8.70 (t, J=6.7 Hz, 0.5H), 8.20 (br s, 1H), 7.314 (d, J=10 Hz, 0.5H), 7.307 (d, J=10 Hz, 0.5H), 6.17 (dd, J=10 and 3.2 Hz, 0.5H), 6.16 (dd, J=10 and 1.7 Hz, 0.5H), 5.92 (s, 1H), 5.17 (t, J=4.8 Hz, 0.5H), 5.03 (d, J=7.3 Hz, 0.5H), 4.81 (s, 1H), 4.72 (d, J=4.4 Hz, 0.5H), 4.62-4.56 (m, 2.5H), 4.51 (d, J=18.9 Hz, 0.5H), 4.47 (d, J=18.6 Hz, 0.5H), 4.30 (s, 1H), 4.20 (d, J=18.9 Hz, 0.5H), 4.16 (d, J=18.6 Hz, 0.5H), 3.74 (s, 2H), 3.15 (s, 2H), 2.58-2.52 (m, 1H), 2.32-2.25 (m, 1H), 2.11-1.95 (m, 2.5H), 1.77-1.70 (m, 2.5H), 1.60-1.39 (m, 5H), 1.383 (s, 0.5H), 1.377 (s, 0.5H), 1.36-1.23 (m, 4H), 1.11-0.92 (m, 2H), 0.87-0.80 (m, 7H) ppm.

### (2S)-2-[(2S)-2-Aminopropanamido]-N-({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)propanamide, TFA salt (PII-5)

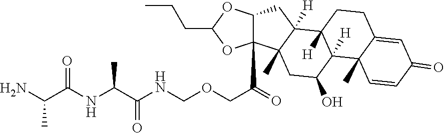

Following a similar procedure used for PII-4, payload PII-5 (10 mg, 44% yield, TFA salt) was obtained from PII-2 and Fmoc-Alanine. ESI m/z: 602.2 (M+1)⁺. ¹H NMR (400 MHz, DMSO_{d6}) δ 8.85 (t, J=6.2 Hz, 0.6H), 8.84 (t, J=5.8 Hz, 0.4H), 8.46 (s, 1H), 7.321 (d, J=10.1 Hz, 0.6H), 7.320 (br s, 3H), 7.313 (d, J=10.1 Hz, 0.4H), 6.170 (dd, J=10.1 and 1.6 Hz, 0.6H), 6.166 (dd, J=10.1 and 1.6 Hz, 0.4H), 5.92 (s, 1H), 5.17 (t, J=4.8 Hz, 0.4H), 5.03 (d, J=7.2 Hz, 0.4H), 4.764 (s, 0.4H), 4.756 (s, 0.6H), 4.73-4.62 (m, 1.6H), 4.60 (t, J=4.4 Hz, 0.6H), 4.57-4.41 (m, 2H), 4.29 (br s, 2H), 4.20 (d, J=18.9 Hz, 0.6H), 4.15 (d, J=18.5 Hz, 0.4H), 3.77-3.67 (m, 1H), 3.08-2.94 (m, 2H), 2.35-2.22 (m, 1H), 2.14-1.91 (m, 2H), 1.83-1.47 (m, 7H), 1.382 (s, 1.8H), 1.377 (s, 1.2H), 1.46-1.31 (m, 1H), 1.31-1.22 (m, 6H), 1.15-0.90 (m, 2H), 0.90-0.76 (m, 6H) ppm.

### 2-Amino-N-(1-{2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}propyl)acetamide (PII-6)

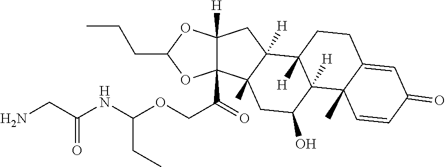

Following General Procedure A for payloads, starting from compound 4d and budesonide, payload PII-6 (40 mg, 4.1% yield) as a white solid was obtained. ESI: 567.2 (M+23)⁺. ¹H NMR (400 MHz, DMSO_{d6}) δ 8.20-8.10 (m, 1H), 7.34-7.29 (m, 1H), 6.16 (dd, J=10 and 1.6 Hz, 1H), 5.92 (s, 1H), 5.15 (t, J=4.8 Hz, 0.4H), 5.10-4.95 (m, 1.8H), 4.83-4.40 (m, 2.8H), 4.29 (br s, 1H), 4.28-4.05 (m, 1H), 3.13 (d, J=7.9 Hz, 2H), 2.60-2.50 (m, 1H), 2.36-2.22 (m, 1H), 2.14-1.87 (m, 3H), 1.81-1.62 (m, 4H), 1.62-1.44 (m, 5H), 1.38 (s, 3H), 1.38-1.18 (m, 3H), 1.13-0.90 (m, 2H), 0.90-0.70 (m, 9H) ppm.

### 2-Amino-N-(1-{2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}-2-phenylethyl)acetamide (PII-7)

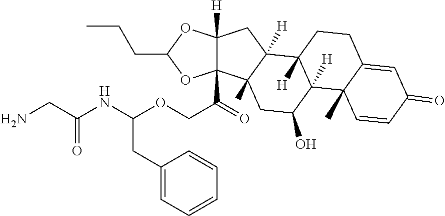

Following General Procedure B for payloads, starting from compound 4e and budesonide, payload PII-7 (80 mg, 29% yield) as a white solid was obtained. ESI m/z: 629 (M+23)⁺. ¹H NMR (400 MHz, DMSO_{d6}) δ 8.45-8.26 (m, 1H), 7.35-7.23 (m, 5H), 7.22-7.16 (m, 1H), 6.16 (dd, J=10.2, 1.9 Hz, 1H), 5.91 (s, 1H), 5.37-5.23 (m, 1H), 5.17-5.12 (m, 0.5H), 5.08 (s, 0.5H), 5.02 (d, J=7.6 Hz, 0.5H), 4.78 (dd, J=9.6, 3.6 Hz, 0.5H), 4.74-4.68 (m, 0.5H), 4.60-4.46 (m, 1.5H), 4.31-4.11 (m, 2H), 3.13-3.01 (m, 3H), 2.93-2.84 (m, 1H), 2.33-2.23 (m, 1H), 2.12-1.92 (m, 2H), 1.79-1.39 (m, 7H), 1.38 (s, 1.5H), 1.37 (s, 1.5H), 1.35-1.22 (m, 2H), 1.14-0.89 (m, 3H), 0.88-0.75 (m, 6H) ppm.

### 2-Amino-N-({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)-N-methylacetamide (PII-8)

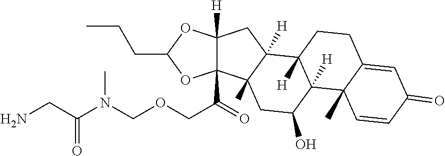

Following General Procedure A for payloads, starting from compound 4f and budesonide, payload PII-8 (64 mg, 12% yield) as a white solid was obtained. ESI m/z: 531 (M+1)⁺. ¹H NMR (500 MHz, DMSO_{d6}) δ 7.35-7.23 (m, 1H), 6.20-6.13 (d, J=10 Hz, 1H), 5.92 (s, 1H), 5.20-4.44 (m, 6H), 4.33-4.08 (m, 2H), 3.441 (s, 0.5H), 3.436 (s, 0.5H), 3.37 (s, 1H), 2.91 (s, 1.5H), 2.895 (s, 0.75H), 2.891 (s, 0.75H), 2.57-2.52 (m, 1H), 2.33-2.24 (m, 1H), 2.13-1.65 (m, 6H), 1.61-1.46 (m, 4H), 1.45-1.41 (m, 1H), 1.383 (s, 1.5H), 1.377 (s, 1.5H), 1.35-1.21 (m, 2H), 1.14-0.91 (m, 2H), 0.89-0.78 (m, 6H) ppm.
Variable-temperature NMR (T=60° C.) indicated the presence of rotamers.

### (2S)-2-Amino-N-({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)-N-methylpropanamide (PII-11)

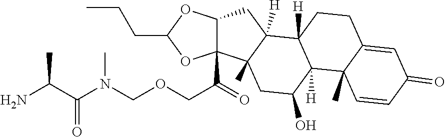 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCN(C)C(=O)[C@H](C)N)O1]

Following the General Procedure A for payloads, starting from compound 4g and budesonide, payload PII-11 (0.13 g, 23% yield) as a white solid was obtained. ESI m/z: 545 (M+1)⁺. ¹H NMR (500 MHz, DMSO_{d6}) δ 7.31 (d, J=10.4 Hz, 1H), 6.20-6.13 (m, 1H), 5.92 (s, 1H), 5.20-5.13 (m, 0.5H), 5.08-5.01 (m, 0.5H), 4.92-4.39 (m, 5H), 4.34-4.07 (m, 2H), 3.88-3.80 (m, 0.5H), 3.76-3.69 (m, 0.5H), 3.02 (s, 1.5H), 2.88 (s, 1.5H), 2.57-2.51 (m, 1H), 2.33-2.25 (m, 1H), 2.12-1.93 (m, 2H), 1.83-1.67 (m, 4H), 1.63-1.23 (m, 10H), 1.12-1.06 (m, 3H), 1.02-0.90 (m, 2H), 0.89-0.78 (m, 6H) ppm.

### 3-Amino-1-({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)piperidin-2-one (PII-12)

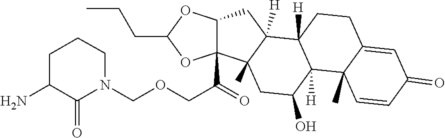 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCN2CCCC(N)C2=O)O1]

Following the General Procedure A for payloads, starting from compound 4h and budesonide, payload PII-12 (19 mg, 15% yield) as a white solid was obtained. ESI m/z: 557 (M+1)⁺. ¹H NMR (400 MHz, DMSO_{d6}) δ 7.33-7.30 (m, 1H), 6.17 (d, J=10 Hz, 1H), 5.92 (s, 1H), 5.20-4.97 (m, 2H), 4.92-4.48 (m, 5H), 4.32-4.12 (m, 2H), 3.27-3.17 (m, 2H), 2.33-2.24 (m, 1H), 2.12-1.92 (m, 6H), 1.82-1.69 (m, 4H), 1.61-1.24 (m, 12H), 0.99-0.92 (m, 1H), 0.87-0.81 (m, 6H) ppm.

### (1S,2S,4R,8S,9S,11S,12S,13R)-8-(2-{[1-(2-Aminoacetyl)pyrrolidin-2-yl]oxy}acetyl)-11-hydroxy-9,13-dimethyl-6-propyl-5,7-dioxapentacyclo[10.8.0.0^{2,9}.0^{4,8}.0^{13,18}]icosa-14,17-dien-16-one (PII-9) (FIG. 2A)

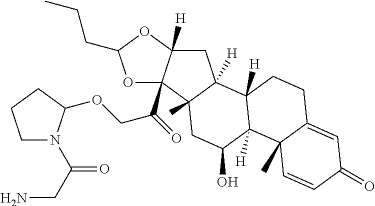 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CN)O1]

To a solution of compound 4J (0.40 g, 1.1 mmol) in DCM (15 mL) was added chlorotrimethylsilane (TMSCl) (0.35 g, 3.3 mmol). The mixture was stirred at RT for 3 hours, which was monitored by LCMS. The volatiles were removed and the residue was redissolved in DCM (6 mL). To the solution were added budesonide (0.94 g, 2.2 mmol) and DIPEA (0.28 g, 2.2 mmol). The mixture was stirred at RT for an hour, which was monitored by LCMS. The resulting mixture was concentrated in vacuo and the residue was purified by reversed phase chromatography (0-100% acetonitrile in aq. ammonium bicarbonate (0.05%)) to give Fmoc-P-GCII-9 (ESI m/z: 801 (M+23)⁺), which was dissolved into DMF (3 mL). To the solution was added piperidine (0.12 g, 1.4 mmol) and the mixture was stirred at RT for an hour until Fmoc was totally removed according to LCMS. The reaction solution was directly purified by prep-HPLC to give payload PII-9 (0.10 g, 17% yield) as a white solid. ESI m/z: 579 (M+23)⁺. ¹H NMR (400 MHz, DMSO_{d6}) δ 7.34-7.29 (m, 1H), 6.16 (dd, J=10.4 and 1.6 Hz, 1H), 5.92 (s, 1H), 5.52-4.99 (m, 2.3H), 4.81-4.47 (m, 3H), 4.35-4.15 (m, 1.7H), 3.47-3.36 (m, 2H), 3.27-3.21 (m, 2H), 2.30-2.28 (m, 1H), 2.09-1.88 (m, 4.7H), 1.84-1.66 (m, 5.7H), 1.61-1.42 (m, 4.6H), 1.38 (s, 3H), 1.38-1.25 (m, 3H), 1.00-0.91 (m, 2H), 0.88-0.77 (m, 6H) ppm.

### (1S,2S,4R,8S,9S,11S,12S,13R)-8-(2-{[1-(2-Aminoacetyl)azetidin-2-yl]oxy}acetyl)-11-hydroxy-9,13-dimethyl-6-propyl-5,7-dioxapentacyclo[10.8.0.0^{2,9}.0^{4,8}.0^{13,18}]icosa-14,17-dien-16-one (PII-13)

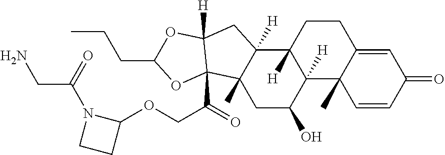

Following the General Procedure A for payloads, starting from compound 4i and budesonide, payload PII-13 (30 mg, 7% yield) as a white solid was obtained. ESI m/z: 543 (M+1)⁺. ¹H NMR (400 MHz, DMSO_{d6}) δ 8.35 (br s, 1H), 8.05 (br s, 1H), 7.40-7.35 (m, 1H), 6.20-6.15 (m, 1H), 5.95 (s, 1H), 5.20-5.15 (m, 0.55H), 5.05-5.00 (m, 0.55H), 4.80-4.60 (m, 3H), 4.40-4.20 (m, 2.45H), 3.80-3.20 (m, 6.45H), 3.20 (s, 2H), 2.25-2.20 (m, 1H), 2.05-1.95 (m, 2H), 1.80-1.30 (m, 13H), 1.20-1.00 (m, 2H), 0.90-0.85 (m, 6H) ppm.

### (2S)-2-Amino-N-({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)propanamide (PIII-5)

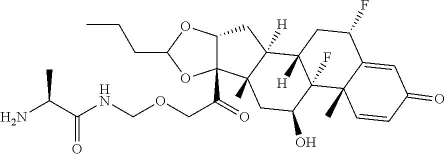

Following the general procedure A for payloads, starting from compound 4b and drug III, payload PIII-5 (90 mg, 61% yield) as a white solid was obtained. ESI m/z: 567 (M+1)⁺.

### 2-Amino-N-({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)acetamide (PIII-1)

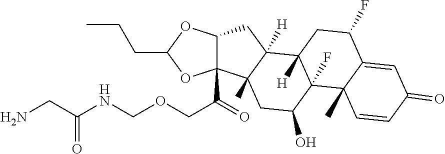

Following General Procedure A for payloads, starting from compound 4a and drug/payload III, payload PIII-1 (0.26 g, 23% yield) as a white solid was obtained. ESI m/z: 553 (M+1)⁺. ¹H NMR (400 MHz, DMSO_{d6}) δ 8.75 (t, J=5.9 Hz, 1H), 7.27 (d, J=10.2 Hz, 1H), 6.30 (dd, J=10.2, 1.7 Hz, 1H), 6.11 (s, 1H), 5.73-5.49 (m, 2H), 5.19 (t, J=4.9 Hz, 0.25H), 5.08 (d, J=7.1 Hz, 0.25H), 4.75 (s, 0.75H), 4.69-4.63 (m, 3.75H), 4.26 (d, J=18.9 Hz, 1H), 4.23-4.13 (m, 1H), 3.17 (s, 2H), 3.00 (br s, 2H), 2.69-2.54 (m, 1H), 2.30-2.18 (m, 1H), 2.08-1.92 (m, 2H), 1.83-1.21 (m, 8H), 1.48 (s, 3H), 0.86 (t, J=7.4 Hz, 3H), 0.81 (s, 3H) ppm.

### 2-Amino-N-{[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}acetamide (PIII-4)

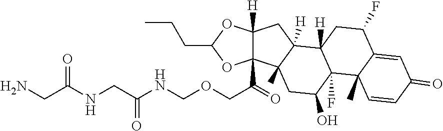 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)CN)O1]

To a solution of compound PIII-1 (55 mg, 0.10 mmol) in DMF (2.0 mL) were added DIPEA (40 mg, 0.31 mmol), Fmoc-Glycine (30 mg, 0.10 mmol) and HATU (50 mg, 0.13 mmol), respectively, at RT. The reaction mixture was stirred at RT for 2 hours, and monitored by LCMS. To the solution was then added piperidine (43 mg, 0.50 mmol), and the mixture was stirred at RT for an hour until Fmoc was removed, which was monitored by LCMS. The mixture was directly purified by reversed phase flash chromatography (0-100% acetonitrile in aq. ammonium bicarbonate (0.08%)) to give compound PIII-4 (50 mg, 82% yield) as a white solid. ESI m/z: 610 (M+1)⁺. ¹H NMR (400 MHz, DMSO_{d6}) δ 8.71 (t, J=6.8 Hz, 1H), 8.21 (br s, 1H), 7.27 (d, J=10.2 Hz, 1H), 6.30 (dd, J=10.2, 1.9 Hz, 1H), 6.11 (s, 1H), 5.72-5.54 (m, 2H), 4.78-4.74 (m, 1H), 4.64 (t, J=4.1 Hz, 1H), 4.61 (d, J=6.8 Hz, 2H), 4.49 (d, J=18.9 Hz, 1H), 4.23 (d, J=18.9 Hz, 1H), 4.21-4.16 (m, 1H), 3.74 (s, 2H), 3.16 (s, 2H), 2.71-2.56 (m, 1H), 2.30-2.20 (m, 1H), 2.04-1.96 (m, 2H), 1.70 (d, J=13.7 Hz, 1H), 1.61-1.52 (m, 4H), 1.49 (s, 3H), 1.44-1.27 (m, 4H), 0.86 (t, J=7.4 Hz, 3H), 0.81 (s, 3H) ppm.

### (1S,4aS,10aR)-N-[(1S,4aS,10aR)-6-{2-[(2-aminoacetamido)methoxy]acetamido}-1,4a-dimethyl-1,2,3,4,4a,9,10,10a-octahydrophenanthrene-1-carbonyl]-6-hydroxy-1,4a-dimethyl-1,2,3,4,4a,9,10,10a-octahydrophenanthrene-1-carboxamide (PIV)

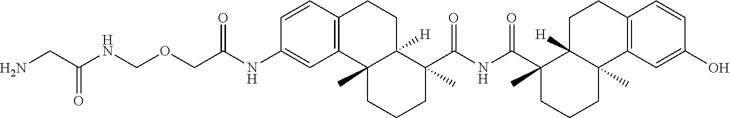 [SMILES: C[C@]1(C(=O)NC(=O)[C@@]2(C)CCC[C@]3(C)c4cc(NC(=O)COCNC(=O)CN)ccc4CC[C@@H]23)CCC[C@]2(C)c3cc(O)ccc3CC[C@@H]12]

To a solution of LXR Agonist 7 (63 mg, 0.12 mmol, see WO 2018/213082 A1) in DMF (5 mL) were added HATU (79 mg, 0.21 mmol) and DIPEA (54 mg, 0.44 mmol) at RT, and the mixture was stirred at RT for 15 minutes before the addition of intermediate 6a (42 mg, 0.11 mmol). The reaction mixture was stirred at RT 4 hours, which was monitored by LCMS. To the mixture was added piperidine (0.2 mL), and the reaction mixture was stirred at RT for an hour until Fmoc was totally removed, which was monitored by LCMS. The mixture was directly purified by prep-HPLC to give payload PIV (34 mg, 47% yield) as a white solid. ESI m/z: 673 (M+1)⁺. ¹H NMR (400 MHz, DMSO_{d6}) δ 9.54 (s, 1H), 8.99 (s, 1H), 8.85 (br s, 1H), 8.11 (s, 1H), 7.59 (d, J=1.5 Hz, 1H), 7.45 (dd, J=8.4, 1.5 Hz, 1H), 6.97 (d, J=8.4 Hz, 1H), 6.82 (d, J=8.4 Hz, 1H), 6.63 (d, J=2.2 Hz, 1H), 6.50 (dd, J=8.4, 2.2 Hz, 1H), 4.66 (d, J=1.6 Hz, 2H), 4.00 (s, 2H), 3.14 (s, 2H), 2.94-2.64 (m, 4H), 2.35-2.32 (m, 1H), 2.32-2.22 (m, 2H), 2.22-2.09 (m, 4H), 1.95-1.78 (m, 4H), 1.68-1.52 (m, 4H), 1.36-1.28 (m, 2H), 1.28 (s, 3H), 1.27 (s, 3H), 1.20-1.09 (m, 2H), 1.01 (s, 3H), 0.99-3H) ppm.

### Synthesis of Payloads PII-9 and PII-10 FIG. 2

Payloads PII-9 and PII-10 were synthesized according to FIG. 2 and the following procedures.

### 9H-Fluoren-9-ylmethyl N-[2-(2-hydroxypyrrolidin-1-yl)-2-oxoethyl]carbamate (4G)

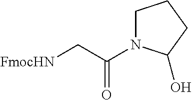

Following General Procedure B for intermediate 4, crude compound 4G (50 mg, 53% yield) was obtained as a white solid. No acetate intermediate was obtained. ESI m/z: 389 (M+23)⁺. ¹H NMR (400 MHz, DMSO_{d6}) δ 7.90 (d, J=7.4 Hz, 2H), 7.73 (d, J=7.5 Hz, 2H), 7.47-7.37 (m, 3H), 7.33 (t, J=7.3 Hz, 2H), 5.86 (br s, 1H), 5.48 (d, J=4.0 Hz, 0.25H), 5.39 (d, J=4.0 Hz, 0.75H), 4.33-4.18 (m, 3H), 3.96 (d, J=6.0 Hz, 1.5H), 3.75 (d, J=6.0 Hz, 0.5H), 3.59-3.33 (m, 1H), 3.22-3.11 (m, 1H), 2.00-1.59 (m, 4H) ppm.

### 9H-Fluoren-9-ylmethyl N-[2-(2-hydroxypiperidin-1-yl)-2-oxoethyl]carbamate (4H)

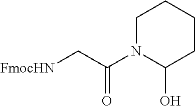

Following General Procedure B for intermediate 4, compound 411 (40 mg, 31% yield) was obtained after prep-HPLC as a white solid. No acetate intermediate was obtained. ESI m/z: 403 (M+²³)⁺.

### (1S,2S,4R,8S,9S,11S,12S,13R)-8-(2-{[1-(2-Aminoacetyl)pyrrolidin-2-yl]oxy}acetyl)-11-hydroxy-9,13-dimethyl-6-propyl-5,7-dioxapentacyclo[10.8.0.0^{2,9}.0^{4,8}.0^{13,18}]icosa-14,17-dien-16-one (PII-9)

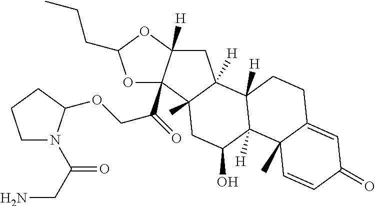 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CN)O1]

To a solution of compound 4G (0.40 g, 1.1 mmol) in DCM (15 mL) was added chlorotrimethylsilane (TMS-Cl) (0.35 g, 3.3 mmol). The mixture was stirred at RT for 3 hours until the alcohol was consumed, as monitored by LCMS. The volatiles were removed and the residue was dissolved again in DCM (6 mL). To the solution were added budesonide (0.94 g, 2.2 mmol) and DIPEA (0.28 g, 2.2 mmol). The mixture was stirred at RT for an hour, and monitored by LCMS. The resulting mixture was concentrated in vacuo and the residue was purified by reversed phase chromatography (0-100% acetonitrile in aq. ammonium bicarbonate (0.05%)) to give Fmoc-P-GCII-9 (ESI m/z: 801 (M+23)⁺), which was dissolved in DMF (3 mL). To the solution was added piperidine (0.12 g, 1.4 mmol) and the mixture was stirred at RT for an hour until Fmoc was totally removed according to LCMS. The reaction solution was directly purified by prep-HPLC to give payload PII-9 (0.10 g, 17% yield) as a white solid. ESI m/z: 579 (M+23)⁺. ¹H NMR (400 MHz, DMSO_{d6}) δ 7.34-7.29 (m, 1H), 6.16 (dd, J=10.4 and 1.6 Hz, 1H), 5.92 (s, 1H), 5.52-4.99 (m, 2.3H), 4.81-4.47 (m, 3H), 4.35-4.15 (m, 1.7H), 3.47-3.36 (m, 2H), 3.27-3.21 (m, 2H), 2.30-2.28 (m, 1H), 2.09-1.88 (m, 4.7H), 1.84-1.66 (m, 5.7H), 1.61-1.42 (m, 4.6H), 1.38 (s, 3H), 1.38-1.25 (m, 3H), 1.00-0.91 (m, 2H), 0.88-0.77 (m, 6H) ppm.

### (1S,2S,4R,8S,9S,11S,12S,13R)-8-(2-{[1-(2-Aminoacetyl)piperidin-2-yl]oxy}acetyl)-11-hydroxy-9,13-dimethyl-6-propyl-5,7-dioxapentacyclo[10.8.0.0^{2,9}.0^{4,8}.0^{13,18}]icosa-14,17-dien-16-one (PII-10)

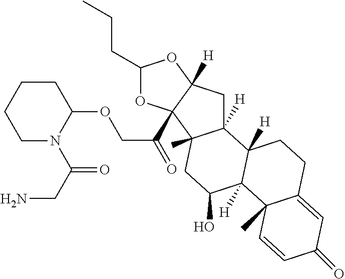 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCCN2C(=O)CN)O1]

Following a similar procedure used for PII-9, payload PII-10 (0.10 g, 20% yield) was obtained as a white solid. ESI m/z: 593 (M+23)⁺.

### Benzyl 2-{[1-(2-{[(9H-fluoren-9-ylmethoxy)carbonyl]amino}acetyl)pyrrolidin-2-yl]oxy}acetate (5b)

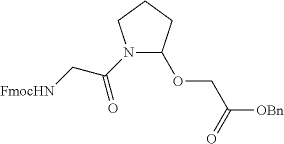

To a solution of compound 4J (0.30 g, 0.82 mmol) in DCM (25 mL) was added chlorotrimethylsilane (TMSCl) (0.27 g, 2.5 mmol). The reaction mixture was stirred at room temperature for 3 hours, which was monitored by LCMS. The resulting mixture was concentrated in vacuo and the residue was diluted with DCM (25 mL). To the solution were added benzyl glycolate (0.27 g, 1.6 mmol) and DIPEA (0.21 g, 1.6 mmol), and the reaction mixture was stirred at room temperature for an hour, which was monitored by LCMS. The resulting mixture was concentrated in vacuo and the residue was purified by reversed phase flash chromatography (0-100% acetonitrile in aq. ammonium bicarbonate (0.05%)) to give compound 5b (0.11 g, 25% yield, purity >99%; and 50 mg at purity 75%) as a white solid. ESI m/z: 537.3 (M+Na)⁺. ¹H NMR (400 MHz, DMSO_{d6}) δ 7.91-7.89 (m, 2H), 7.74-7.69 (m, 2H), 7.63-7.48 (m, 1H), 7.42-7.25 (m, 9H), 5.51-5.09 (m, 2H), 4.35-4.21 (m, 5H), 4.00-3.77 (m, 2H), 3.52-3.38 (m, 2H), 3.30-3.18 (m, 1H), 2.19-1.64 (m, 4H) ppm.

### 2-{[1-(2-{[(9H-Fluoren-9-ylmethoxy)carbonyl]amino}acetyl)pyrrolidin-2-yl]oxy}acetic acid (6b)

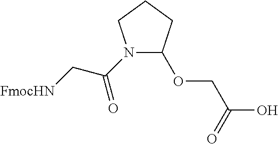

To a solution of compound 5b (89 mg, 0.17 mmol) in methanol (3 mL) and THE (7 mL) was added wet palladium on carbon (10% Pd, 20 mg) under nitrogen. The mixture was degassed, purged with hydrogen, and stirred under a hydrogen balloon at room temperature for 2 hours, which was monitored by LCMS. The reaction mixture was filtered through Celite and the filtrate was concentrated in vacuo. The residue was purified by reversed phase flash chromatography (0-100% acetonitrile in aq. ammonium bicarbonate (0.05%)) to give compound 6b (36 mg, 49% yield) as a white solid. ESI m/z: 447.1 (M+Na)⁺.

### Synthesis of Linker-Payloads

### Synthesis of Linker-Payloads L1-P-#

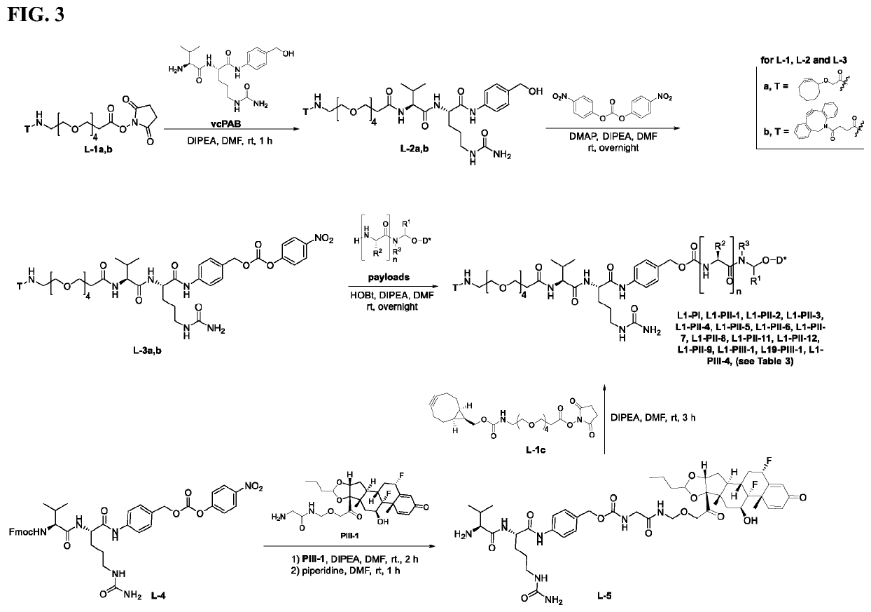

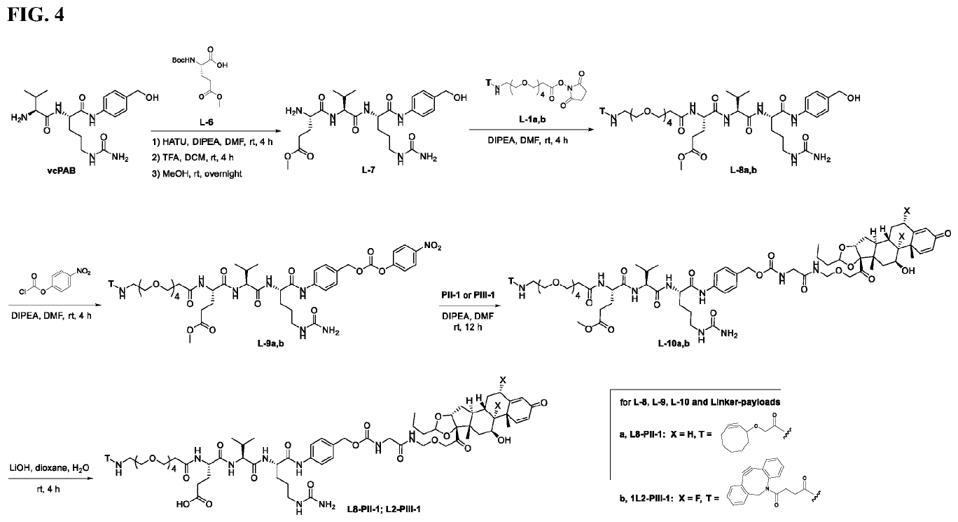

Linker-payloads L1-PI, L1-PII-1, L1-PII-2, L1-PII-3, L1-PII-4, L1-PII-5, L1-PII-6, L1-PII-7, L1-PII-8, L1-PII-11, L1-PII-12, L1-PII-9, L1-PIII-1, L19-PIII-1, and L1-PIII-4, were synthesized according to FIG. 3 and the following procedures; linker-payloads L8-PII-1 and L2-PIII-1 were synthesized according to FIG. 4 and the following procedures. The intermediate L-3b was synthesized according to WO 2018/089373. The linkers vcPAB (CAS: 1497404-26-8), L-1a (CAS: 1427004-19-0), and L-1b (CAS: 2101206-50-0) L-1c (CAS: 1702356-19-1) were commercially available.

### Synthesis of Intermediate L-3a (FIG. 3)

### N-[(1S)-1-{[(1S)-4-(Carbamoylamino)-1-{[4-(hydroxymethyl)phenyl]carbamoyl}butyl]carbamoyl}-2-methylpropyl]-1-[2-(cyclooct-2-yn-1-yloxy)acetamido]-3,6,9,12-tetraoxapentadecan-15-amide (L-2a)

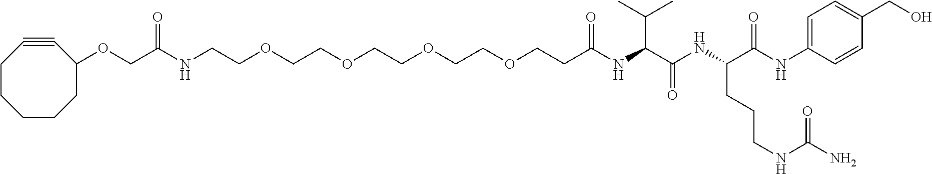 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(CO)cc1]

To a solution of compound L-la (0.17 g, 0.33 mmol) in DMF (10 mL) were added DIPEA (0.13 g, 1.0 mmol) and vcPAB (0.13 g, 0.34 mmol) successively, and the reaction mixture was stirred at room temperature for an hour, which was monitored by LCMS. The resulting mixture was directly purified by reversed phase flash chromatography (0-80% acetonitrile in water) to give compound L-2a (0.18 g, 70% yield) as colorless oil. ESI m/z: 791.3 (M+H)⁺. ¹H NMR (400 MHz, DMSO_{d6}) δ 9.91 (s, 1H), 8.11 (d, J=8.4 Hz, 1H), 7.89 (d, J=8.8 Hz, 1H), 7.61 (t, J=5.6 Hz, 1H), 7.55 (d, J=8.4 Hz, 2H), 7.23 (d, J=8.4 Hz, 2H), 5.98 (t, J=5.6 Hz, 1H), 5.42 (s, 2H), 5.10 (br s, 1H), 4.43 (s, 2H), 4.39-4.37 (m, 1H), 4.30-4.21 (m, 2H), 3.87 (d, J=14.8 Hz, 1H), 3.75 (d, J=14.8 Hz, 1H), 3.62-3.58 (m, 2H), 3.50-3.46 (m, 12H), 3.43 (t, J=6.0 Hz, 2H), 3.27-3.22 (m, 2H), 3.06-2.92 (m, 2H), 2.41-2.32 (m, 2H), 2.26-2.05 (m, 3H), 1.99-1.66 (m, 6H), 1.62-1.55 (m, 3H), 1.44-1.35 (m, 3H), 0.89 (d, J=6.8 Hz, 3H), 0.83 (d, J=6.8 Hz, 3H) ppm.

### {4-[(2S)-5-(Carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl 4-nitrophenyl carbonate (L-3a)

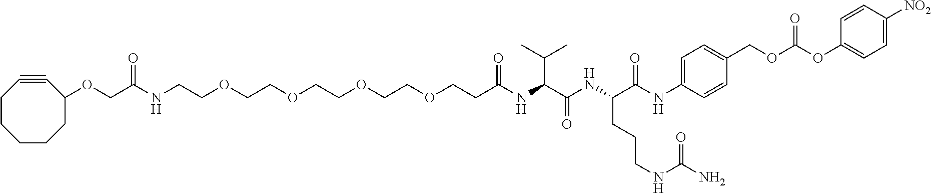 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(COC(=O)Oc2ccc([N+](=O)[O-])cc2)cc1]

A suspension of compound L-2a (80 mg, 0.10 mmol), DMAP (12 mg, 0.10 mmol), and DIPEA (26 mg, 0.20 mmol) in dry DMF (5 mL) was stirred at room temperature for 10 minutes before the addition of bis(4-nitrophenyl) carbonate (61 mg, 0.20 mmol). The reaction mixture was stirred at room temperature for 2 hours, which was monitored by LCMS. The resulting mixture was directly purified by reversed phase flash chromatography (0-80% acetonitrile in water) to give compound L-3a (53 mg, 55% yield) as a white solid. ESI m/z: 956.3 (M+H)⁺.

### General Procedure for vcPAB Linker-Payloads L1-PI, L1-PII-1, L1-PII-2, L1-PII-3, L1-PII-4, L1-PII-5, L1-PII-6, L1-PII-7, L1-PII-8, L1-PII-11, L1-PII-12, L1-PII-9, L1-PIII-1, L19-PIII-1, and L1-PIII-4 (FIG. 3)

To a solution of payload (1.0 equiv.) in DMF (5-10 mM) were added compound L-3a,b (1.0 equiv.), HOBt (0-0.05 equiv.) and DIPEA (2.0 equiv.), and the mixture was stirred at RT overnight, which was monitored by LCMS. The resulting mixture was directly purified by prep-HPLC to give vcPAB linker-payloads L1-PI, L1-PII-1, L1-PII-2, L1-PII-3, L1-PII-4, L1-PII-5, L1-PII-6, L1-PII-7, L1-PII-8, L1-PII-11, L1-PII-12, L1-PII-9, L1-PIII-1, L 19-PIII-1, and L1-PIII-4 (11-68% yield; see Table 3) as white solids.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-{[({2-[(1R,2S,10S,11S,13R,14R,15S,17S)-1-fluoro-14,17-dihydroxy-2,13,15-trimethyl-5-oxotetracyclo[8.7.0.0^{2,7}.0^{11,15}]heptadeca-3,6-dien-14-yl]-2-oxoethoxy}methyl)carbamoyl]methyl}carbamate (L1-PI)

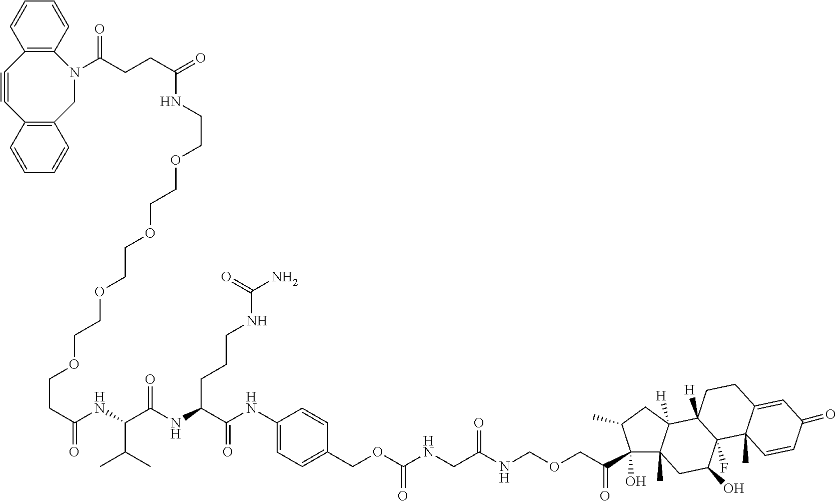 [SMILES: CC(C)[C@H](NC(=O)CCOCCOCCOCCOCCNC(=O)CCC(=O)N1Cc2ccccc2C#Cc2ccccc21)C(=O)N[C@@H](CCCNC(N)=O)C(=O)Nc1ccc(COC(=O)NCC(=O)NCOCC(=O)[C@@]2(O)[C@H](C)C[C@H]3[C@@H]4CCC5=CC(=O)C=C[C@]5(C)[C@@]4(F)[C@@H](O)C[C@@]32C)cc1]

Following the general procedure for vc PAB linker-payloads, starting from Payload PI and compound L-3b, linker-payload L1-PI (8.2 mg, 17% yield) was obtained as a white solid. ESI m/z: 710 (M/2+1)⁺. ¹H NMR (400 MHz, DMSO_{d6}) δ 10.01 (s, 1H), 8.72 (t, J=6.7 Hz, 1H), 8.14 (d, J=7.1 Hz, 1H), 7.89 (d, J=8.0 Hz, 1H), 7.78 (t, J=5.6 Hz, 1H), 7.68 (d, J=8.0 Hz, 1H), 7.65-7.54 (m, 3H), 7.54-7.42 (m, 4H), 7.42-7.21 (m, 6H), 6.22 (d, J=10.0 Hz, 1H), 6.05-5.96 (m, 2H), 5.43 (s, 2H), 5.26 (s, 1H), 5.06-4.90 (m, 4H), 4.63-4.48 (m, 3H), 4.44-4.31 (m, 1H), 4.27-4.08 (m, 3H), 3.66-3.54 (m, 4H), 3.52-3.35 (m, 12H), 3.33-3.24 (m, 2H), 3.14-2.84 (m, 4H), 2.70-2.53 (m, 2H), 2.48-1.88 (m, 10H), 1.82-1.53 (m, 4H), 1.48 (s, 3H), 1.47-1.27 (m, 4H), 1.23 (s, 2H), 1.10-1.00 (m, 1H), 0.87 (s, 3H), 0.86 (d, J=6.5 Hz, 3H), 0.82 (d, J=6.7 Hz, 3H), 0.78 (d, J=7.2 Hz, 3H) ppm.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-{[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamate (L1-PII-1)

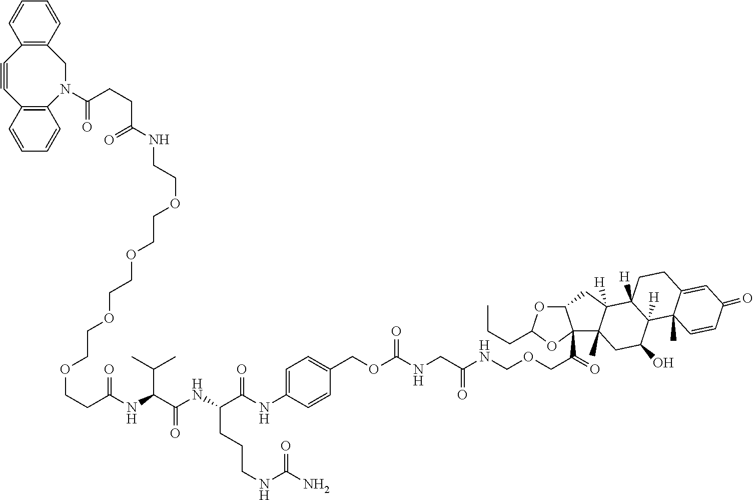

Following the general procedure for vcPAB linker-payloads, starting from Payload PII-1 and compound L-3b, linker-payload L1-PII-1 (5.0 mg, 11% yield) was obtained as a white solid. ESI m/z: 729 (M/2+1)⁺. ¹H NMR (400 MHz, DMSO_{d6}) δ 10.00 (s, 1H), 8.70-8.60 (m, 1H), 8.20-8.10 (m, 1H), 7.90-7.50 (m, 5H), 7.50-7.20 (m, 10H), 6.15-6.10 (m, 1H), 6.05-6.00 (m, 1H), 5.95 (s, 1H), 5.50 (s, 2H), 5.05-5.00 (m, 3H), 4.80-4.30 (m, 8H), 3.60-3.55 (m, 4H), 3.50-3.45 (m, 14H), 3.40-3.30 (m, 4H), 3.10-2.80 (m, 4H), 2.60-2.55 (m, 1H), 2.40-2.20 (m, 5H), 2.00-1.90 (m, 4H), 1.80-1.60 (m, 4H), 1.60-1.50 (m, 5H), 1.40-1.30 (m, 8H), 1.00-0.80 (m, 14H) ppm.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-[({[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)methyl]carbamate (L1-PII-2)

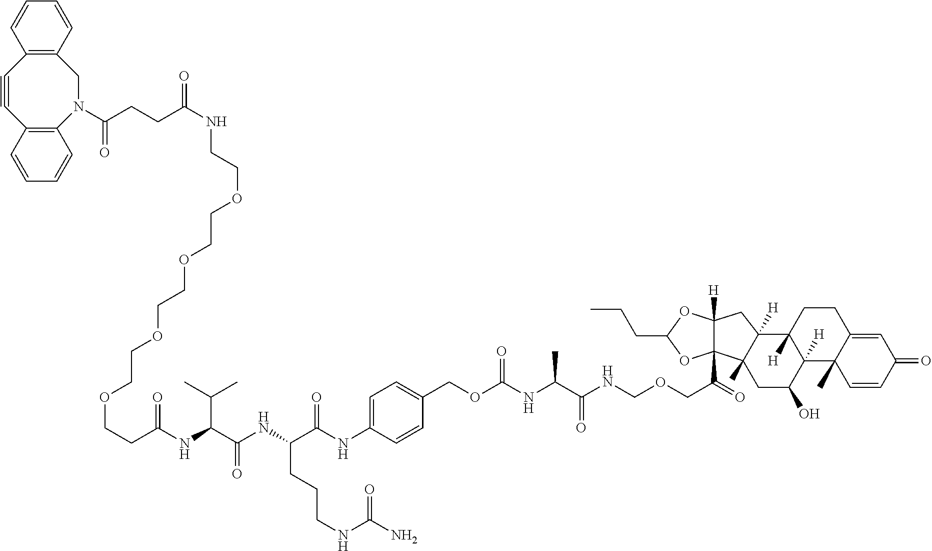

Following the general procedure for vcPAB linker-payloads, starting from Payload PII-2 and compound L-3b, linker-payload L1-PII-2 (1.0 mg, 18% yield) was obtained as a white solid. ESI m/z: 1041 (M-Bud)⁺, 736 (M/2+1)⁺, 521 [(M-Bud)/2]⁺. ¹H NMR (400 MHz, DMSO_{d6}) δ 9.95 (s, 1H), 8.60-8.55 (m, 1H), 8.00 (d, J=10.0 Hz, 1H), 7.75-7.70 (d, J=6.4 Hz, 1H), 7.65-7.20 (m, 15H), 6.05-6.00 (m, 1H), 5.90-5.85 (m, 1H), 5.80 (s, 1H), 5.30 (s, 2H), 5.10-4.60 (m, 4H), 4.55-4.10 (m, 11H), 3.90-3.85 (m, 2H), 3.50-3.45 (m, 4H), 3.40-3.30 (m, 14H), 3.20-3.15 (m, 2H), 3.00-2.80 (m, 3H), 2.50-2.45 (m, 2H), 2.30-2.10 (m, 3H), 2.00-1.80 (m, 4H), 1.75-1.50 (m, 4H), 1.45-1.25 (m, 5H), 1.20-1.05 (m, 6H), 1.05-1.00 (m, 3H), 0.85-0.75 (m, 12H) ppm.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-{[(1-{2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}propyl)carbamoyl]methyl}carbamate (L1-PII-3)

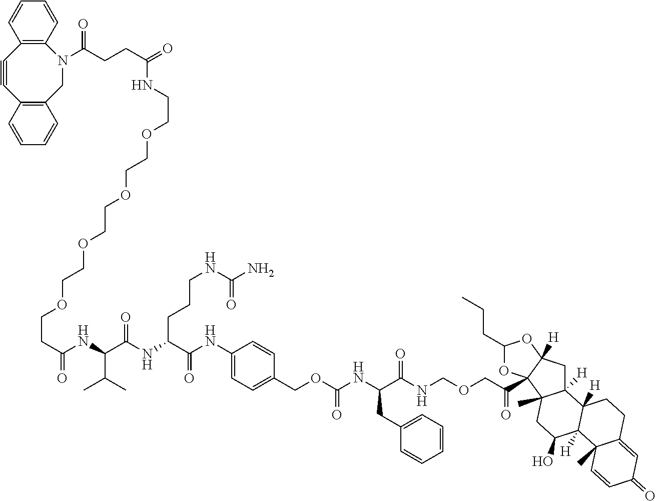 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)[C@@H](Cc2ccccc2)NC(=O)OCc2ccc(NC(=O)[C@@H](CCCNC(N)=O)NC(=O)[C@H](NC(=O)CCOCCOCCOCCOCCNC(=O)CCC(=O)N3Cc4ccccc4C#Cc4ccccc43)C(C)C)cc2)O1]

Following the general procedure for vcPAB linker-payloads, starting from Payload PII-3 and compound L-3b, linker-payload L1-PII-3 (0.10 g, 47% yield) was obtained as a white solid. ESI m/z: 774 (M/2+1)⁺, 1116.5 (M-Bud)⁺, 558.9 [(M-Bud)/2]⁺. ¹H NMR (400 MHz, DMSO_{d6}) δ 9.96 (s, 1H), 8.93-8.91 (m, 1H), 8.11 (d, J=7.2 Hz, 1H), 7.87 (d, J=8.4 Hz, 1H), 7.75 (t, J=4.8 Hz, 1H), 7.69-7.45 (m, 9H), 7.39-7.26 (m, 9H), 7.24-7.16 (m, 3H), 6.16 (d, J=10.0 Hz, 1H), 5.97-5.92 (m, 2H), 5.41 (s, 2H), 5.17-5.01 (m, 2H), 4.91-4.82 (m, 2H), 4.73-4.54 (m, 5H), 4.49-4.13 (m, 6H), 3.62-3.57 (m, 3H), 3.47-3.45 (m, 13H), 3.11-2.92 (m, 5H), 2.78-2.72 (m, 1H), 2.40-2.20 (m, 4H), 2.07-1.94 (m, 3H), 1.80-1.67 (m, 4H), 1.60-1.51 (m, 4H), 1.43-1.24 (m, 9H), 0.99-0.82 (m, 15H) ppm.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-[({[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)methyl]carbamate (L1-PII-4)

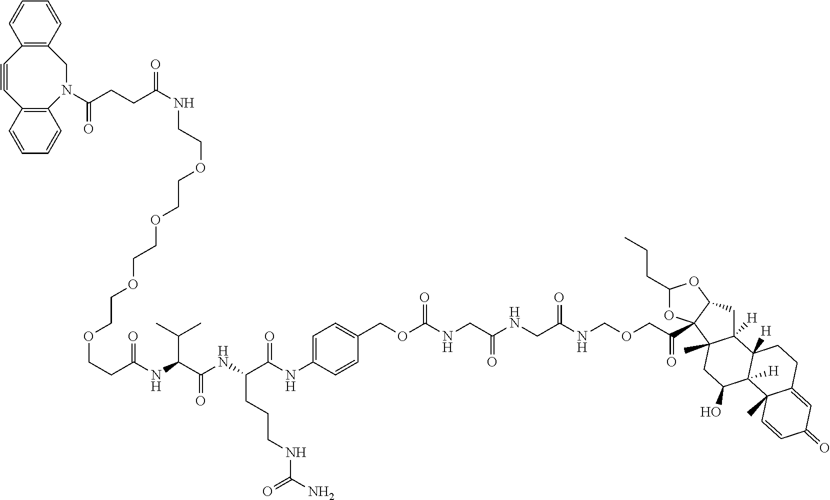

Following the general procedure for vcPAB linker-payloads, starting from Payload PII-4 and compound L-3b, linker-payload L1-PII-4 (45 mg, 68% yield) was obtained as a white solid. ESI m/z: 757.5 (M/2+1)⁺. ¹H NMR (400 MHz, DMSO_{d6}) δ 9.99 (s, 1H), 8.69-8.65 (m, 1H), 8.23-8.20 (m, 1H), 8.13 (d, J=7.0 Hz, 1H), 7.87 (d, J=8.5 Hz, 1H), 7.76 (t, J=6.0 Hz, 1H), 7.68-7.67 (m, 1H), 7.62-7.58 (m, 3H), 7.51-7.42 (m, 4H), 7.39-7.28 (m, 6H), 6.17-6.15 (m, 1H), 5.97 (t, J=6.0 Hz, 1H), 5.91 (s, 1H), 5.41 (s, 2H), 5.16 (t, J=5.5 Hz, 0.5H), 5.04-5.01 (m, 1.5H), 4.96 (s, 2H), 4.73-4.71 (m, 1.5H), 4.61-4.58 (m, 2.5H), 4.49 (t, J=19.0 Hz, 1H), 4.40-4.35 (m, 1H), 4.30-4.28 (m, 1H), 4.24-4.14 (m, 2H), 3.72 (d, J=5.5 Hz, 2H), 3.67 (d, J=6.0 Hz, 2H), 3.62-3.56 (m, 3H), 3.47-3.45 (m, 10H), 3.44-3.43 (m, 2H), 3.31-3.28 (m, 3H), 3.10-3.05 (m, 2H), 3.04-3.00 (m, 1H), 2.97-2.90 (m, 1H), 2.60-2.54 (m, 1H), 2.47-2.44 (m, 1H), 2.39-2.34 (m, 1H), 2.30-2.20 (m, 2H), 2.08-1.94 (m, 4H), 1.78-1.66 (m, 4H), 1.59-1.49 (m, 5H), 1.45-1.39 (m, 2H), 1.37 (d, J=3.0 Hz, 3H), 1.35-1.30 (m, 2H), 1.28-1.21 (m, 2H), 0.99-0.91 (m, 1H), 0.86-0.80 (m, 12H) ppm.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-[({[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)methyl]carbamate (L1-PII-5)

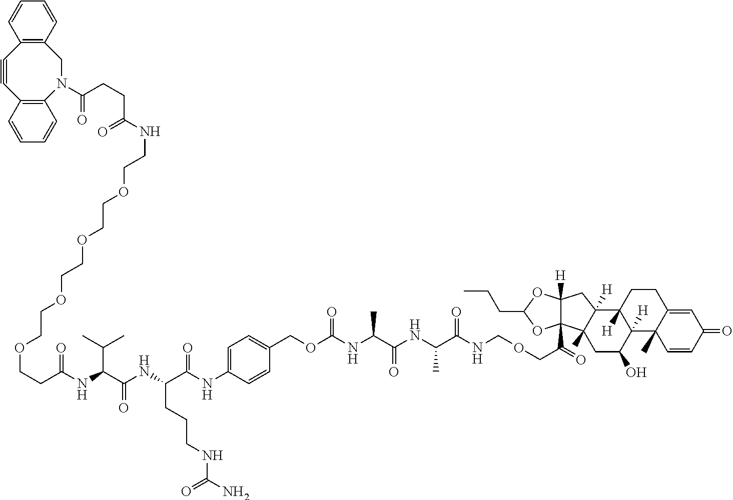

Following the general procedure for vcPAB linker-payloads, starting from Payload PII-5 and compound L-3b, linker-payload L1-PII-5 (4 mg, 25% yield) was obtained as a white solid. ESI m/z: 771.5 (M/2+1)⁺, 556.3 [(M-Bud)]⁺. ¹H NMR (400 MHz, DMSO_{d6}) δ 9.99 (s, 1H), 8.79-8.63 (m, 1H), 8.13 (d, J=7.4 Hz, 1H), 8.04 (d, J=7.2 Hz, 1H), 7.88 (d, J=8.6 Hz, 1H), 7.77 (t, J=5.6 Hz, 1H), 7.71-7.54 (m, 4H), 7.54-7.42 (m, 3H), 7.42-7.21 (m, 7H), 6.16 (d, J=10.1 Hz, 1H), 5.99 (t, J=5.5 Hz, 1H), 5.92 (s, 1H), 5.42 (s, 2H), 5.24-4.87 (m, 4H), 4.80-4.00 (m, 11H), 3.66-3.54 (m, 3H), 3.53-3.41 (m, 12H), 3.32-3.27 (m, 2H), 3.13-2.87 (m, 4H), 2.68-2.45 (m, 2H), 2.42-2.17 (m, 4H), 2.08 (s, 1H), 2.05-1.90 (m, 4H), 1.83-1.64 (m, 4H), 1.64-1.39 (m, 7H), 1.377 (s, 1.8H), 1.371 (s, 1.2H), 1.36-1.26 (m, 2H), 1.23 (d, J=7.0 Hz, 3H), 1.19 (d, J=7.0 Hz, 3H), 1.12-0.89 (m, 2H), 1.12-0.91 (m, 12H) ppm.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-{[(1-{2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}propyl)carbamoyl]methyl}carbamate (L1-PII-6)

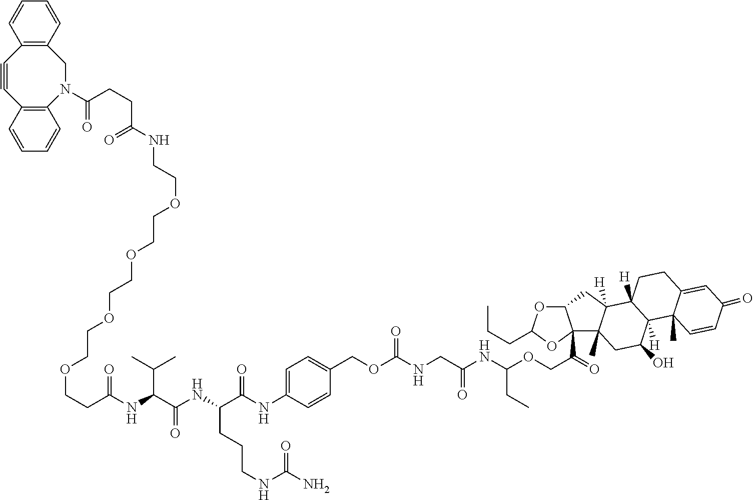 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC(CC)NC(=O)CNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)O1]

Following the general procedure for vcPAB linker-payloads, starting from Payload PII-6 and compound L-3b, linker-payload L1-PII-6 (20 mg, 50% yield) was obtained as a white solid. ESI m/z: 1055 (M-Bud)⁺, 528 [(M-Bud)/2]⁺, ¹H NMR (500 MHz, DMSO_{d6}) δ 9.98 (s, 1H), 8.31-8.19 (m, 1H), 8.11 (d, J=7.4 Hz, 1H), 7.86 (d, J=8.6 Hz, 1H), 7.75 (t, J=5.6 Hz, 1H), 7.68 (d, J=7.2 Hz, 1H), 7.63-7.55 (m, 3H), 7.52-7.43 (m, 3H), 7.43-7.32 (m, 3H), 7.32-7.21 (m, 4H), 6.15 (d, J=10.0 Hz, 1H), 5.97 (t, J=5.6 Hz, 1H), 5.91 (s, 1H), 5.40 (s, 2H), 5.20-4.88 (m, 5H), 4.75-4.08 (m, 7H), 3.70-3.53 (m, 5H), 3.51-3.40 (m, 12H), 3.30-3.27 (m, 2H), 3.13-2.89 (m, 4H), 2.65-2.53 (m, 1H), 2.47-2.43 (m, 1H), 2.41-2.18 (m, 4H), 2.04-1.97 (m, 4H), 1.80-1.64 (m, 5H), 1.63-1.47 (m, 6H), 1.47-1.21 (m, 8H), 1.11-0.90 (m, 2H), 0.89-0.76 (m, 15H) ppm.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-{[(1-{2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}-2-phenylethyl)carbamoyl]methyl}carbamate (L1-PII-7)

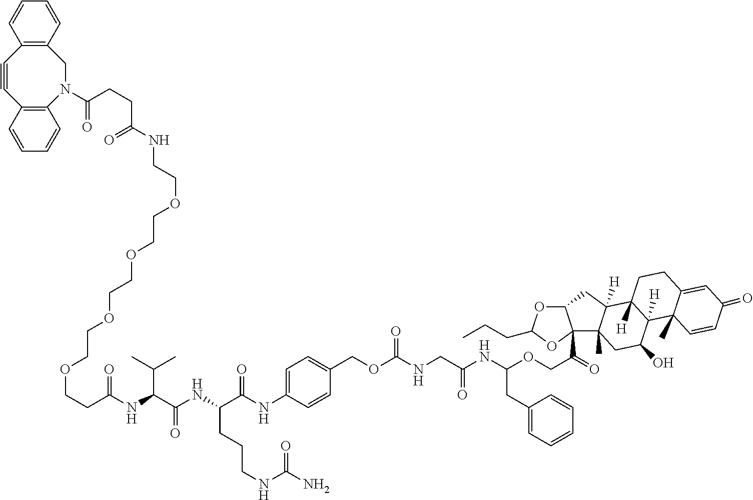

Following the general procedure for vcPAB linker-payloads, starting from Payload PII-7 and compound L-3b, linker-payload L1-PII-7 (25 mg, 33% yield) was obtained as a white solid. ESI m/z: 1116.5 (M-Bud)⁺. ¹H NMR (500 MHz, DMSO_{d6}) δ 9.98 (s, 1H), 8.46 (d, J=9.7 Hz, 1H), 8.12 (d, J=8.3 Hz, 1H), 7.87 (d, J=8.9 Hz, 1H), 7.75 (t, J=5.6 Hz, 1H), 7.68 (d, J=7.8 Hz, 1H), 7.64-7.55 (m, 3H), 7.53-7.42 (m, 3H), 7.41-7.31 (m, 3H), 7.31-7.14 (m, 9H), 6.14 (d, J=9.6 Hz, 1H), 5.97 (br s, 1H), 5.91 (s, 1H), 5.41 (s, 2H), 5.31-5.21 (m, 1H), 5.18-5.11 (m, 0.5H), 5.05-5.00 (m, 1.5H), 4.99-4.89 (m, 2H), 4.76-4.63 (m, 1H), 4.59-4.47 (m, 2H), 4.38 (m, 1H), 4.31-4.13 (m, 3H), 3.67-3.54 (m, 3H), 3.51-3.40 (m, 12H), 3.33-3.25 (m, 2H), 3.13-2.83 (m, 6H), 2.65-2.54 (m, 1H), 2.41-2.18 (m, 5H), 2.10-1.91 (m, 4H), 1.80-1.64 (m, 4H), 1.63-1.20 (m, 15H), 1.11-0.89 (m, 2H), 0.88-0.76 (m, 12H) ppm.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-{[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)(methyl)carbamoyl]methyl}carbamate (L1-PII-8)

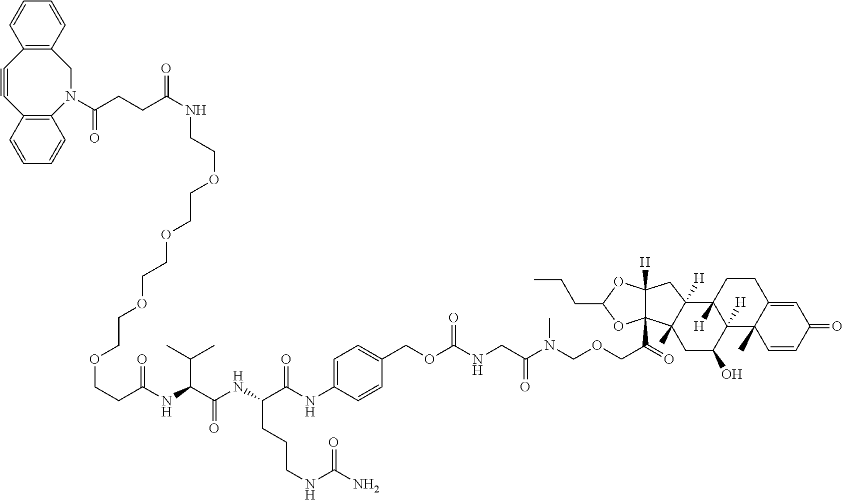

Following the general procedure for vcPAB linker-payloads, starting from Payload PII-8 and compound L-3b, linker-payload L1-PII-8 (60 mg, 44% yield) was obtained as a white solid. ESI m/z: 736 (M/2+1)⁺. ¹H NMR (500 MHz, DMSO_{d6}) δ 10.00 (s, 1H), 8.14 (d, J=7.5 Hz, 1H), 7.89 (d, J=8.5 Hz, 1H), 7.76 (d, J=5.9 Hz, 1H), 7.68 (d, J=7.1 Hz, 1H), 7.64-7.54 (m, 3H), 7.52-7.42 (m, 3H), 7.40-7.18 (m, 5H), 6.19-6.12 (m, 1H), 6.00 (t, J=5.1 Hz, 1H), 5.92 (s, 1H), 5.41 (s, 2H), 5.20-5.13 (m, 0.5H), 5.08-4.99 (m, 1.5H), 4.96 (s, 2H), 4.85-4.08 (m, 10H), 4.02-3.95 (m, 1H), 3.92-3.85 (m, 1H), 3.64-3.54 (m, 3H), 3.51-3.40 (m, 12H), 3.33-3.27 (m, 2H), 2.91-2.86 (m, 5H), 2.90 (s, 2H), 2.65-2.53 (m, 1H), 2.47-2.42 (m, 1H), 2.41-2.32 (m, 1H), 2.32-2.19 (m, 2H), 2.13-1.91 (m, 4H), 1.81-1.65 (m, 5H), 1.63-1.47 (m, 5H), 1.46-1.40 (m, 2H), 1.38 (s, 3H), 1.36-1.21 (m, 4H), 1.15-0.90 (m, 2H), 0.89-0.77 (m, 12H) ppm.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-[(1S)-1-[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)(methyl)carbamoyl]ethyl]carbamate (L1-PII-11)

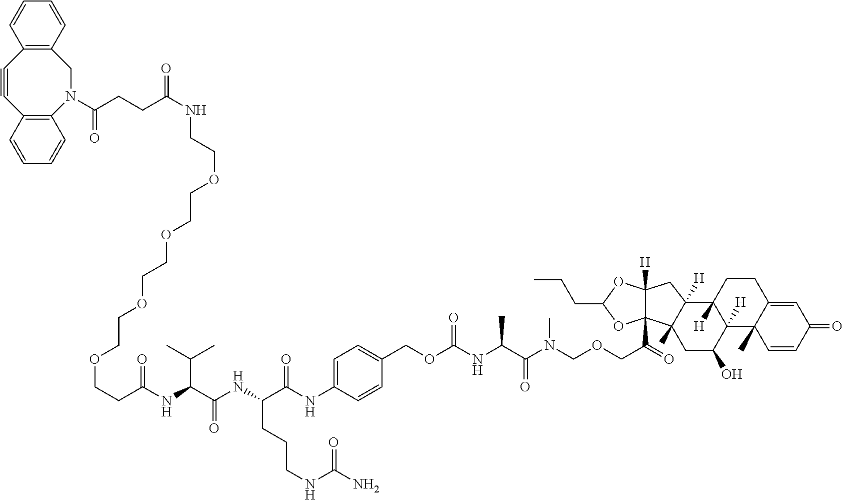

Following the general procedure for vcPAB-linker-payloads, starting from Payload PII-11 and compound L-3b, linker-payload L1-PII-11 (50 mg, 61% yield) was obtained as a white solid. ESI m/z: 743 (M/2+1)⁺. ¹H NMR (400 MHz, DMSO_{d6}) δ 9.98 (s, 1H), 8.12 (d, J=7.6 Hz, 1H), 7.87 (d, J=8.4 Hz, 1H), 7.76 (t, J=5.6 Hz, 1H), 7.80-7.74 (m, 1H), 7.71-7.66 (m, 1H), 7.64-7.54 (m, 3H), 7.53-7.43 (m, 3H), 7.40-7.24 (m, 6H), 6.15 (d, J=13.2 Hz, 1H), 5.97 (t, J=5.6 Hz, 1H), 5.92 (s, 1H), 5.41 (s, 2H), 5.20-4.83 (m, 5H), 4.78-4.68 (m, 2H), 4.67-4.50 (m, 2H), 4.49-4.34 (m, 2H), 4.33-4.12 (m, 3H), 3.67-3.55 (m, 3H), 3.52-3.41 (m, 12H), 3.31-3.27 (m, 2H), 3.12-2.84 (m, 7H), 2.62-2.53 (m, 2H), 2.47-2.43 (m, 1H), 2.41-2.34 (m, 1H), 2.33-2.19 (m, 2H), 2.11-1.91 (m, 4H), 1.82-1.67 (m, 4H), 1.63-1.49 (m, 4H), 1.47-1.40 (m, 2H), 1.39-1.35 (m, 3H), 1.25-1.15 (m, 4H), 1.01-0.89 (m, 2H), 0.89-0.79 (m, 12H) ppm.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-[1-({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)-2-oxopiperidin-3-yl]carbamate (L1-PII-12)

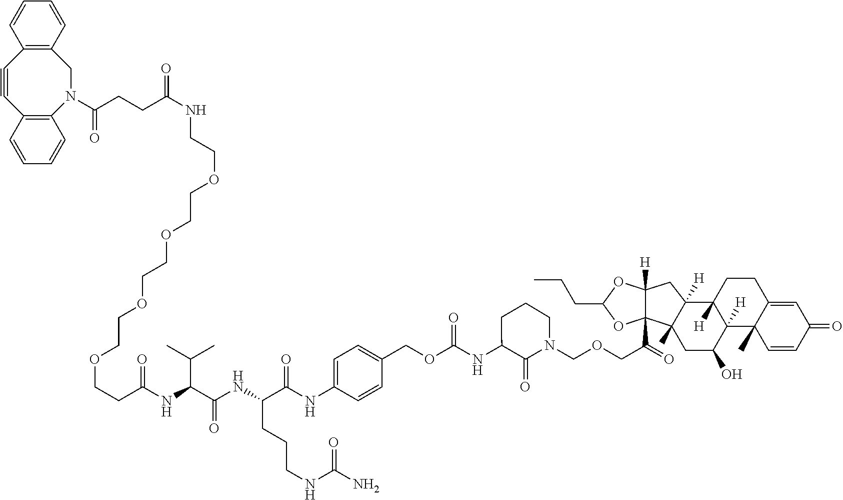

Following the general procedure for vcPAB-linker-payloads, starting from Payload PII-12 and compound L-3b, linker-payload L1-PII-12 (5.0 mg, 19% yield) was obtained as a white solid. ESI m/z: 749 (M/2+1)⁺. ¹H NMR (400 MHz, DMSO_{d6}) δ 9.99 (s, 1H), 8.11 (d, J=7.0 Hz, 1H), 7.87 (d, J=8.5 Hz, 1H), 7.79-7.74 (m, 1H), 7.69-7.67 (m, 1H), 7.63-7.58 (m, 3H), 7.51-7.25 (m, 9H), 6.17-6.14 (m, 1H), 6.00-5.97 (m, 1H), 5.92 (s, 1H), 5.41 (s, 2H), 5.33-5.15 (m, 1H), 5.04-5.01 (m, 1H), 4.95-4.47 (m, 7H), 4.40-4.18 (m, 4H), 4.00-3.93 (m, 1H), 3.62-3.56 (m, 3H), 3.47-3.42 (m, 11H), 3.32-3.26 (m, 5H), 3.12-2.93 (m, 4H), 2.62-2.55 (m, 1H), 2.50-2.20 (m, 4H), 2.02-1.68 (m, 13H), 1.60-1.28 (m, 14H), 0.99-0.90 (m, 2H), 0.86-0.81 (m, 12H) ppm.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-[2-(2-{2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}pyrrolidin-1-yl)-2-oxoethyl]carbamate (L1-PII-9)

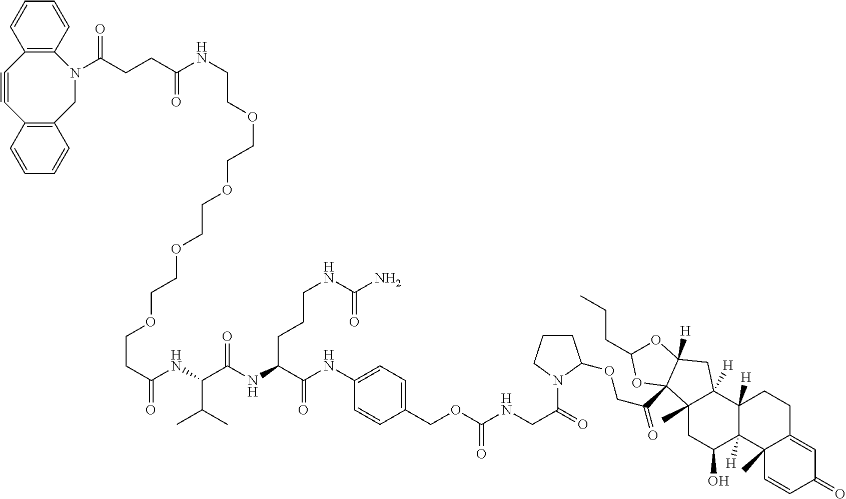 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)O1]

Following the general procedure for vcPAB linker-payloads, starting from Payload PII-9 and compound L-3b, linker-payload L1-PII-9 (42 mg, 44% yield) was obtained as a white solid. ESI m/z: 1066 (M-Bud)⁺, 534 [(M-Bud)/2]⁺. ¹H NMR (500 MHz, DMSO_{d6}) δ 10.04 (s, 1H), 8.20 (d, J=7.8 Hz, 1H), 7.93 (d, J=7.6 Hz, 1H), 7.76 (t, J=5.7 Hz, 1H), 7.68 (d, J=7.5 Hz, 1H), 7.64-7.56 (m, 3H), 7.53-7.43 (m, 3H), 7.40-7.23 (m, 7H), 6.18-6.16 (m, 1H), 6.03-6.02 (m, 1H), 5.92 (s, 1H), 5.42 (s, 2H), 5.32 (s, 1H), 5.04-4.94 (m, 4H), 4.74-4.50 (m, 4H), 4.38-4.37 (m, 1H), 4.29-4.21 (m, 3H), 3.79-3.78 (m, 1H), 3.62-3.55 (m, 3H), 3.47-3.45 (m, 12H), 3.31-3.19 (m, 2H), 3.10-2.93 (m, 4H), 2.64-2.54 (m, 1H), 2.48-2.45 (m, 1H), 2.40-2.20 (m, 2H), 2.11-1.88 (m, 7H), 1.85-1.64 (m, 7H), 1.60-1.40 (m, 8H), 1.38 (s, 3H), 1.36-1.19 (m, 4H), 1.00-0.91 (m, 2H), 0.89-0.75 (m, 12H) ppm.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-[2-(2-{2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}piperidin-1-yl)-2-oxoethyl]carbamate (L1-PII-10)

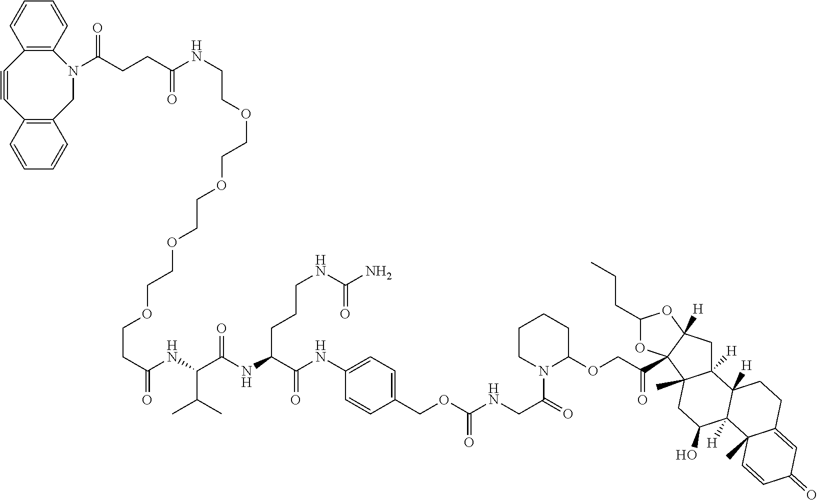 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCCN2C(=O)CNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)O1]

Following the general procedure for vcPAB linker-payloads, starting from Payload PII-10 and compound L-3b, linker-payload L1-PII-10 (50 mg, 50% yield) was obtained as a white solid. ESI m/z: 1080 (M-Bud)⁺, 541 [(M-Bud)/2]⁺.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-{[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamate (L1-PIII-1)

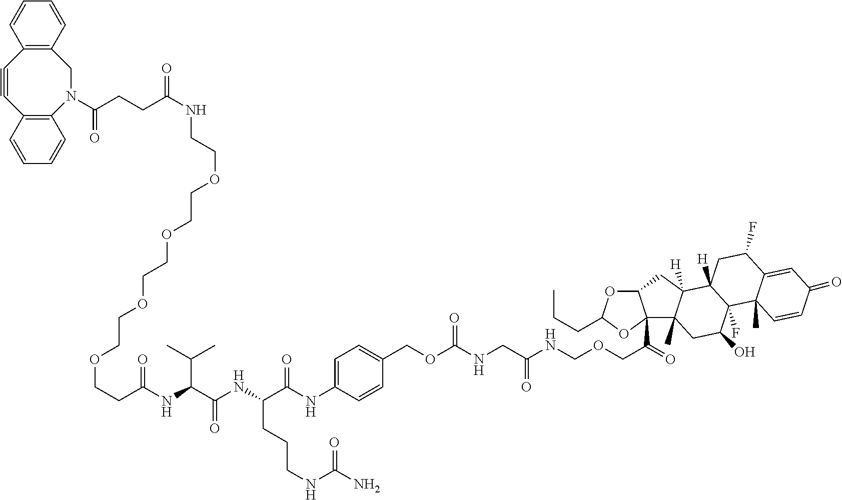 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)O1]

Following the general procedure for vcPAB linker-payloads, starting from Payload PIII-1 and compound L-3b, linker-payload L1-PIII-1 (23 mg, 58% yield) was obtained as a white solid. ESI m/z: 747 (M/2+1)⁺. ¹H NMR (400 MHz, DMSO_{d6}) δ 10.08 (s, 1H), 8.83-8.71 (m, 1H), 8.15 (d, J=6.6 Hz, 1H), 7.89 (d, J=8.7 Hz, 1H), 7.78 (t, J=5.4 Hz, 1H), 7.68 (d, J=7.7 Hz, 1H), 7.65-7.55 (m, 3H), 7.52-7.44 (m, 4H), 7.41-7.22 (m, 6H), 6.29 (d, J=10.2 Hz, 1H), 6.11 (s, 1H), 5.99 (t, J=5.0 Hz, 1H), 5.73-5.36 (m, 4H), 5.22-4.93 (m, 3H), 4.78-4.45 (m, 4H), 4.42-4.33 (m, 1H), 4.28-4.14 (m, 3H), 3.67-3.54 (m, 5H), 3.52-3.41 (m, 12H), 3.29 (t, J=5.9 Hz, 2H), 3.18-2.89 (m, 4H), 2.72-2.42 (m, 3H), 2.42-2.18 (m, 4H), 2.10-1.89 (m, 4H), 1.82-1.65 (m, 3H), 1.63-1.51 (m, 4H), 1.48 (s, 3H), 1.47-1.19 (m, 6H), 0.90-0.75 (m, 12H) ppm.

### {4-[(2S)-2-[(2S)-2-Amino-3-methylbutanamido]-5-(carbamoylamino)pentanamido]phenyl}methyl N-{[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamate

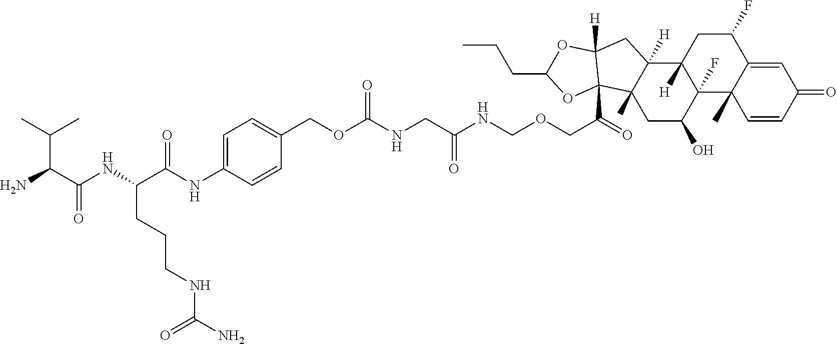

To a solution of Fmoc-vcPAB-PNP (L-4) (60 mg, 78 μmol) in DMF (2 mL) were added payload PIII-1 (44 mg, 80 μmol) and DIPEA (31 mg, 0.24 mmol), and the mixture was stirred at room temperature for 2 hours, which was monitored by LCMS. To the resulting mixture was added piperidine (34 mg, 0.40 mmol), and the mixture was stirred at room temperature for an hour until Fmoc was totally removed, which was monitored by LCMS. The reaction mixture was directly purified by reversed phase flash chromatography (0-100% acetonitrile in aq. ammonium bicarbonate (0.05%)) to give compound L-5 (45 mg, 60% yield) as a yellow solid. ESI m/z: 958 (M+1)⁺.

### (1R,8S,9R)-Bicyclo[6.1.0]non-4-yn-9-ylmethyl N-(14-{[(1S)-1-{[(1S)-4-(carbamoylamino)-1-[(4-{[({[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)oxy]methyl}phenyl)carbamoyl]butyl]carbamoyl}-2-methylpropyl]carbamoyl}-3,6,9,12-tetraoxatetradecan-1-yl)carbamate (L19-PIII-1)

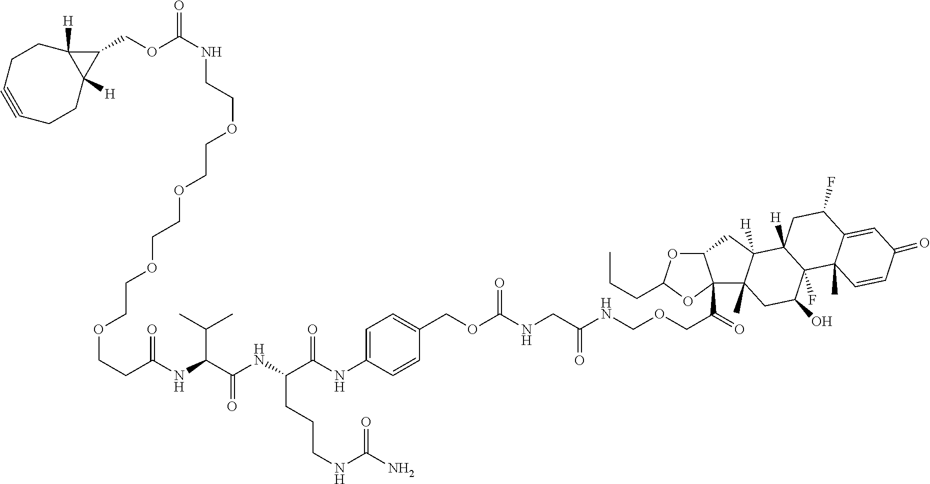 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)OCc2ccc(NC(=O)[C@H](CCCNC(N)=O)NC(=O)[C@@H](NC(=O)CCOCCOCCOCCOCCNC(=O)OC[C@@H]3[C@@H]4CCC#CCC[C@@H]43)C(C)C)cc2)O1]

To a solution of compound L-5 (30 mg, 31 μmol) in DMF (2 mL) were added compound L-1c (34 mg, 63 μmol) and DIPEA (12 mg, 93 μmol), and the reaction mixture was stirred at room temperature for 2 hours, which was monitored by LCMS. The resulting mixture was directly purified by reversed phase flash chromatography (0-100% acetonitrile in aq. ammonium bicarbonate (0.05%)) to give linker-payload L19-PIII-1 (8.0 mg, 14% yield) as a white solid. ESI m/z: 691 (M/2+1)⁺. ¹H NMR (400 MHz, DMSO_{d6}) δ 10.00 (s, 1H), 8.75 (t, J=6.8 Hz, 1H), 8.14 (d, J=7.6 Hz, 1H), 7.89 (d, J=8.4 Hz, 1H), 7.60 (d, J=8.4 Hz, 2H), 7.47 (t, J=6.0 Hz, 1H), 7.31-7.25 (m, 3H), 7.12-7.09 (m, 1H), 6.29 (dd, J=10.4 and 1.6 Hz, 1H), 6.11 (s, 1H), 5.99-5.97 (m, 1H), 5.72-5.56 (m, 1H), 5.48-5.46 (m, 1H), 5.42 (s, 2H), 4.96 (s, 2H), 4.76-4.75 (m, 1H), 4.67-4.49 (m, 4H), 4.41-4.36 (m, 1H), 4.27-4.19 (m, 3H), 4.04-4.02 (m, 2H), 3.67-3.56 (m, 4H), 3.50-3.45 (m, 12H), 3.41-3.38 (m, 2H), 3.14-3.09 (m, 2H), 3.05-2.92 (m, 2H), 2.43-2.33 (m, 1H), 2.27-2.13 (m, 7H), 2.07-1.94 (m, 3H), 1.72-1.69 (m, 2H), 1.59-1.22 (m, 17H), 0.87-0.82 (m, 15H) ppm.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-[({[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)methyl]carbamate (L1-PIII-4)

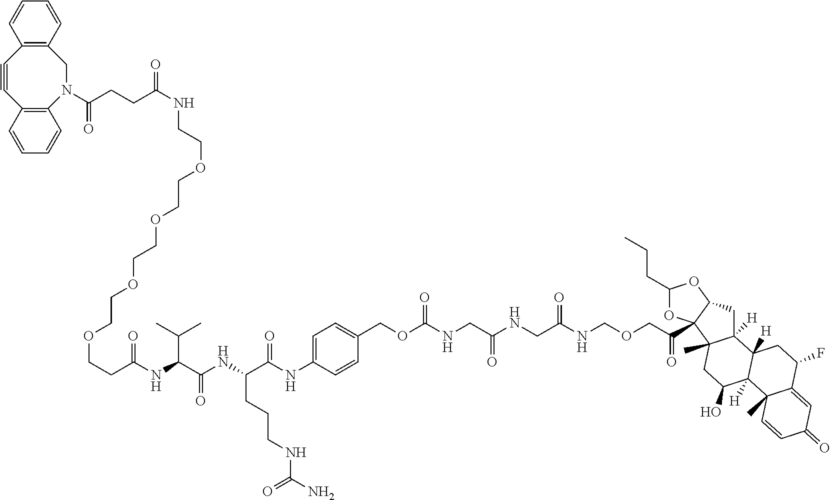 [SMILES: CCCC1O[C@@H]2C[C@H]3[C@@H]4C[C@H](F)C5=CC(=O)C=C[C@]5(C)[C@H]4[C@@H](O)C[C@]3(C)[C@]2(C(=O)COCNC(=O)CNC(=O)CNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)O1]

Following the general procedure for vcPAB linker-payloads, starting from Payload PIII-4 and compound L-3b, linker-payload L1-PIII-4 (30 mg, 43% yield) was obtained as a white solid. ESI m/z: 775 (M/2+1)⁺. ¹H NMR (400 MHz, DMSO_{d6}) δ 10.00 (s, 1H), 8.71-8.64 (m, 1H), 8.26-8.20 (m, 1H), 8.13 (d, J=7.0 Hz, 1H), 7.88 (d, J=8.8 Hz, 1H), 7.79-7.73 (m, 1H), 7.68 (d, J=7.5 Hz, 1H), 7.64-7.57 (m, 3H), 7.53-7.43 (m, 4H), 7.39-7.33 (m, 2H), 7.32-7.25 (m, 4H), 6.29 (d, J=8.5 Hz, 1H), 6.11 (s, 1H), 6.01-5.95 (m, 1H), 5.72-5.66 (m, 0.5H), 5.60-5.53 (m, 0.5H), 5.48 (s, 1H), 5.42 (s, 2H), 5.03 (d, J=14.1 Hz, 1H), 4.96 (s, 2H), 4.75 (s, 1H), 4.67-4.57 (m, 3H), 4.50 (d, J=18.7 Hz, 1H), 4.41-4.34 (m, 1H), 4.28-4.17 (m, 3H), 3.72 (d, J=5.3 Hz, 2H), 3.67 (d, J=5.8 Hz, 2H), 3.64-3.56 (m, 3H), 3.52-3.41 (m, 13H), 3.31-3.28 (m, 2H), 3.12-2.93 (m, 4H), 2.63-2.57 (m, 1H), 2.41-2.36 (m, 1H), 2.28-2.20 (m, 2H), 2.06-1.93 (m, 4H), 1.80-1.65 (m, 3H), 1.62-1.51 (m, 4H), 1.48 (s, 3H), 1.47-1.20 (m, 7H), 0.95-0.75 (m, 12H) ppm.

### {4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-({[({[(4bS,8S,8aR)-8-{[(1S,4aS,10aR)-6-hydroxy-1,4a-dimethyl-1,2,3,4,4a,9,10,10a-octahydrophenanthrene-1-carbonyl]carbamoyl}-4b,8-dimethyl-4b,5,6,7,8,8a,9,10-octahydrophenanthren-3-yl]carbamoyl}methoxy)methyl]carbamoyl}methyl)carbamate (L1-PIV)

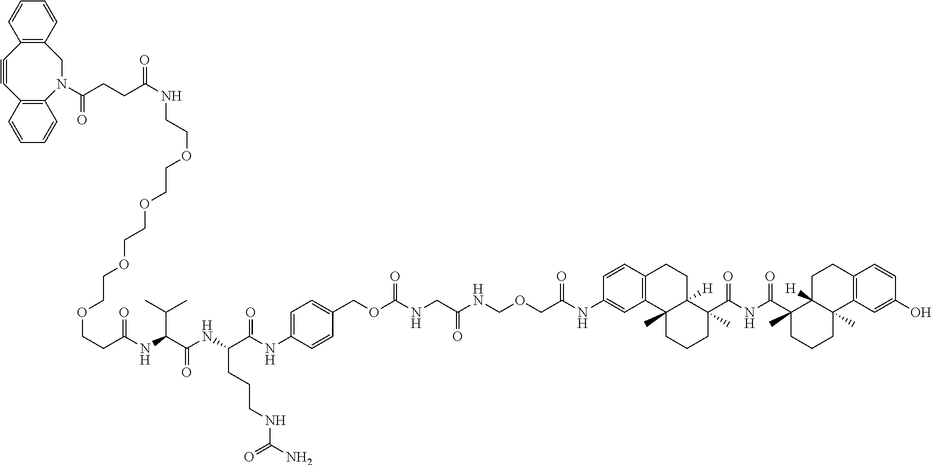 [SMILES: CC(C)[C@H](NC(=O)CCOCCOCCOCCOCCNC(=O)CCC(=O)N1Cc2ccccc2C#Cc2ccccc21)C(=O)N[C@@H](CCCNC(N)=O)C(=O)Nc1ccc(COC(=O)NCC(=O)NCOCC(=O)Nc2ccc3c(c2)[C@@]2(C)CCC[C@](C)(C(=O)NC(=O)[C@@]4(C)CCC[C@]5(C)c6cc(O)ccc6CC[C@@H]45)[C@@H]2CC3)cc1]

Following the general procedure for vcPAB-linker-payloads, starting from Payload PIV and compound L-3b, linker-payload L1-PIV (20 mg, 53% yield) was obtained as a white solid. ESI m/z: 807 (M/2+1)⁺. ¹H NMR (400 MHz, DMSO_{d6}) δ 10.00 (s, 1H), 9.53 (s, 1H), 8.99 (s, 1H), 8.92-8.83 (m, 1H), 8.19-8.07 (m, 2H), 7.88 (d, J=8.1 Hz, 1H), 7.77 (t, J=5.6 Hz, 1H), 7.68 (d, J=6.0 Hz, 1H), 7.64-7.56 (m, 4H), 7.53-7.25 (m, 9H), 6.96 (d, J=8.5 Hz, 1H), 6.81 (d, J=8.3 Hz, 1H), 6.63 (s, 1H), 6.50 (d, J=7.9 Hz, 1H), 6.02-5.94 (m, 1H), 5.42 (s, 2H), 5.02 (d, J=14 Hz, 1H), 4.95 (s, 2H), 4.65 (d, J=6.5 Hz, 2H), 4.43-4.32 (m, 1H), 4.26-4.19 (m, 1H), 4.00 (s, 2H), 3.70-3.54 (m, 5H), 3.50-3.41 (m, 12H), 3.31-3.26 (m, 1H), 3.13-2.54 (m, 8H), 2.41-2.08 (m, 11H), 2.06-1.52 (m, 14H), 1.47-1.08 (m, 12H), 1.00 (s, 3H), 0.98 (s, 3H), 0.86 (d, J=6.8 Hz, 3H), 0.82 (d, J=6.8 Hz, 3H) ppm.

### Synthesis of Linker-Payload L2-PIII-1

Linker-payload L8-PII-1 and L2-PIII-1 were synthesized according to FIG. 4 and the following procedures.

### Methyl (4S)-4-amino-4-{[(1S)-1-{[(1S)-4-(carbamoylamino)-1-{[4-(hydroxymethyl)phenyl]carbamoyl}butyl]carbamoyl}-2-methylpropyl]carbamoyl}butanoate (L-7)

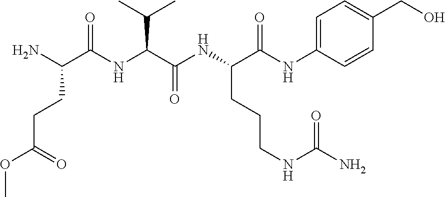 [SMILES: COC(=O)CC[C@H](N)C(=O)N[C@H](C(=O)N[C@@H](CCCNC(N)=O)C(=O)Nc1ccc(CO)cc1)C(C)C]

To a solution of compound L-6 (26 mg, 0.10 mmol) in DMF (1 mL) were added HATU (38 mg, 0.10 mmol) and DIPEA (26 mg, 0.20 mmol). The reaction mixture was stirred at RT for 10 min before the addition of vcPAB (38 mg, 0.10 mmol). The mixture was stirred at RT for 16 hours, and monitored by LCMS. The resulting mixture was subjected to prep-HPLC to give impure Boc-L-7 (ESI m/z: 623 (M+1)⁺), which was dissolved in DCM (3 mL). To the solution was added TFA (0.3 mL), and the reaction mixture was stirred at RT for 4 hours until Boc was totally removed according to LCMS. The mixture was concentrated in vacuo and the residue was dissolved in methanol (2 mL). After stirring at RT overnight, the mixture was concentrated in vacuo. The residue was purified by prep-HPLC to give compound L-7 (23 mg, 43% yield) as colorless oil. ESI m/z: 523 (M+1)⁺.

### Methyl (4S)-4-{[(1S)-1-{[(1S)-4-(carbamoylamino)-1-{[4-(hydroxymethyl)phenyl]carbamoyl}butyl]carbamoyl}-2-methylpropyl]carbamoyl}-4-{1-[2-(cyclooct-2-yn-1-yloxy)acetamido]-3,6,9,12-tetraoxapentadecan-15-amido}butanoate (L-8a)

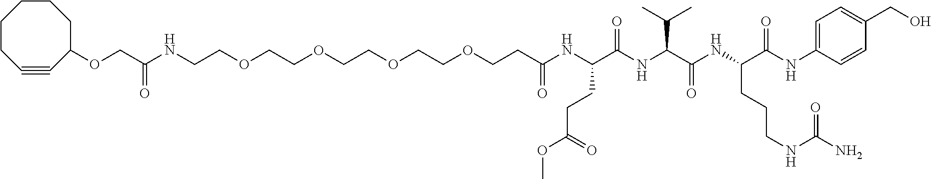 [SMILES: COC(=O)CC[C@H](NC(=O)CCOCCOCCOCCOCCNC(=O)COC1C#CCCCCC1)C(=O)N[C@H](C(=O)N[C@@H](CCCNC(N)=O)C(=O)Nc1ccc(CO)cc1)C(C)C]

To a solution of compound L-7 (0.10 g, 0.19 mmol) in DMF (5 mL) were added compound L-la (0.10 mg, 0.19 mmol) and DIPEA (49 mg, 0.38 mmol), and the reaction mixture was stirred at room temperature for 4 hours, which was monitored by LCMS. The resulting mixture was directly purified by prep-HPLC to give compound L-8a (0.11 g, 63% yield) as a white solid. ESI m/z: 934 (M+1)⁺.

### Methyl (4S)-4-{[(1S)-1-{[(1S)-4-(carbamoylamino)-1-[(4-{[({[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)oxy]methyl}phenyl)carbamoyl]butyl]carb amoyl}-2-methylpropyl]carbamoyl}-4-{1-[2-(cyclooct-2-yn-1-yloxy)acetamido]-3,6,9,12-tetraoxapentadecan-15-amido}butanoate (L-10a)

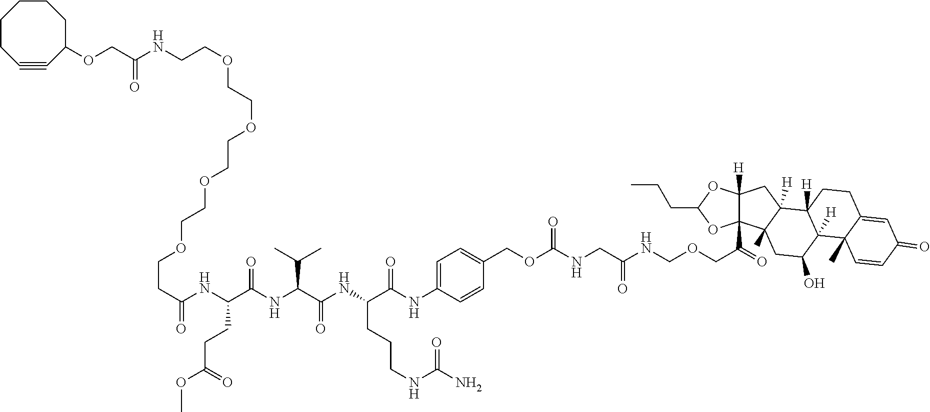

To a solution of compound L-8a (0.10 g, 0.11 mmol) in DMF (3 mL) were added 4-nitrophenyl chloroformate (24 mg, 0.12 mmol) and DIPEA (70 mg, 54 mmol). The reaction mixture was stirred at room temperature for 4 hours, which was monitored by LCMS. The solution containing compound L-9a was used for the next step directly. ESI m/z: 550.5 (M/2+1)⁺. To this solution (0.50 mL) were added PII-1 (9.5 mg, 18 μmol) and DIPEA (4.7 mg, 36 μmol). The reaction mixture was stirred at room temperature for 4 hours, which was monitored by LCMS. The mixture was purified by prep-HPLC to give compound L-10a (14 mg, 76% yield) as a white solid. ESI m/z: 1499 (M+23)⁺.

### (4S)-4-{[(1S)-1-{[(1S)-4-(Carbamoylamino)-1-[(4-{[({[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)oxy]methyl}phenyl)carbamoyl]butyl]carb amoyl}-2-methylpropyl]carbamoyl}-4-{1-[2-(cyclooct-2-yn-1-yloxy)acetamido]-3,6,9,12-tetraoxapentadecan-15-amido}butanoic acid (L8-PII-1)

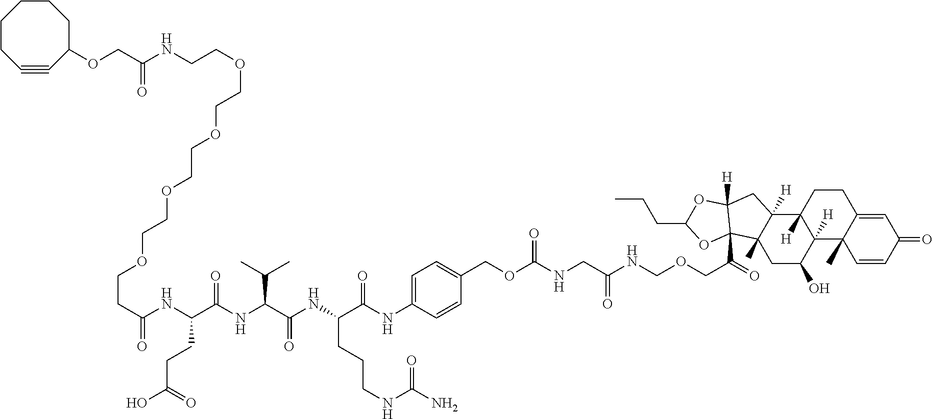

To a solution of compound L-10a (30 mg, 20 μmol) in dioxane (0.6 mL) and water (0.2 mL) was added lithium hydroxide (3.0 mg, 0.12 mmol), and the reaction mixture was stirred at room temperature for 18 hours, which was monitored by LCMS. The residue was purified by prep-HPLC to give L8-PII-1 (24 mg, 75% yield) as a white solid. ESI m/z: 1463 (M+1)⁺. ¹H NMR (400 MHz, DMSO_{d6}) δ 10.03 (s, 1H), 8.74 (d, J=4.8 Hz, 1H), 8.22 (s, 1H), 8.09 (d, J=8.0 Hz, 1H), 7.75 (d, J=8.5 Hz, 1H), 7.59 (d, J=8.3 Hz, 3H), 7.46 (t, J=6.1 Hz, 1H), 7.30 (dd, J=11.4, 6.0 Hz, 3H), 6.16 (d, J=10.1 Hz, 1H), 5.92 (s, 1H), 5.46-5.31 (m, 2H), 5.17-4.96 (m, 3H), 4.71-4.58 (m, 4H), 4.53-4.44 (m, 1H), 4.40-4.25 (m, 4H), 4.20 (m, 2H), 3.87 (d, J=14.8 Hz, 1H), 3.75 (d, J=14.7 Hz, 1H), 3.64-3.56 (m, 4H), 3.42 (t, J=6.0 Hz, 3H), 3.25 (m, 3H), 3.14-2.84 (m, 3H), 2.44-1.80 (m, 18H), 1.80-1.67 (m, 6H), 1.63-1.40 (m, 10H), 1.40-1.32 (m, 6H), 1.28-1.22 (m, 6H), 0.96 (m, 2H), 0.90-0.75 (m, 14H) ppm.

### Methyl (4S)-4-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-4-{[(1S)-1-{[(1S)-4-(carbamoylamino)-1-{[4-(hydroxymethyl)phenyl]carbamoyl}butyl]carbamoyl}-2-methylpropyl]carbamoyl}butanoate (L-8b)

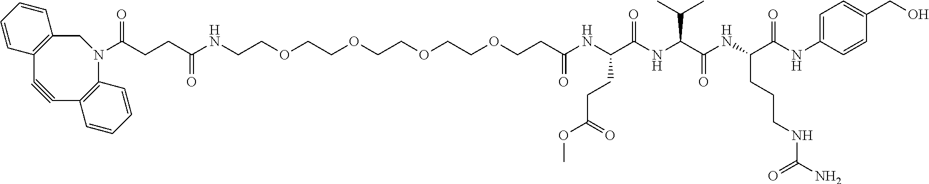 [SMILES: COC(=O)CC[C@H](NC(=O)CCOCCOCCOCCOCCNC(=O)CCC(=O)N1Cc2ccccc2C#Cc2ccccc21)C(=O)N[C@H](C(=O)N[C@@H](CCCNC(N)=O)C(=O)Nc1ccc(CO)cc1)C(C)C]

Following a similar procedure as L-8a, except substituting L-1b for L-1a, compound L-8b (70 mg, 47% yield) was obtained as a white solid. ESI m/z: 529 (M/2+1)⁺.

### Methyl (4S)-4-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-4-{[(1S)-1-{[(1S)-4-(carbamoylamino)-1-[(4-{[({[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)oxy]methyl}phenyl)carbamoyl]butyl]carbamoyl}-2-methylpropyl]carbamoyl}butanoate (L-10b)

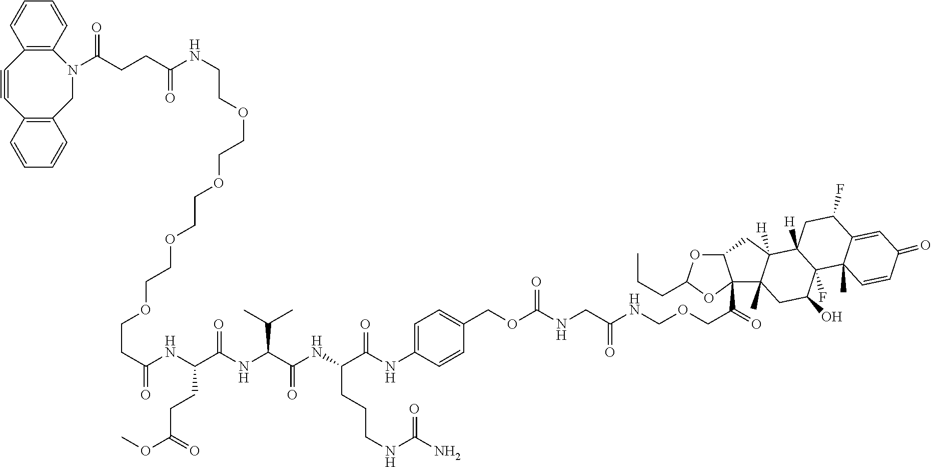 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)OCc2ccc(NC(=O)[C@H](CCCNC(N)=O)NC(=O)[C@@H](NC(=O)[C@H](CCC(=O)OC)NC(=O)CCOCCOCCOCCOCCNC(=O)CCC(=O)N3Cc4ccccc4C#Cc4ccccc43)C(C)C)cc2)O1]

To a solution of compound L-8b (7.0 mg, 6.6 μmol) in DMF (0.5 mL) were added 4-nitrophenyl chloroformate (1.3 mg, 6.7 μmol) and DIPEA (1.7 mg, 13 μmol). The reaction mixture was stirred at RT for 4 hours, which was monitored by LCMS. The solution containing compound L-9b was used in the next step without purification. ESI m/z: 612 (M/2+1)⁺. To this solution (2.4 μmol (calc.)) were added PIII-1 (1.4 mg, 2.4 μmol) and DIPEA (0.6 mg, 4.8 μmol). The reaction mixture was stirred at RT for 4 hours, which was monitored by LCMS. The mixture was purified by prep-HPLC to give compound L-10b (3 mg, 76% yield) as a white solid. ESI m/z: 819 (M/2+1)⁺.

### (4S)-4-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-4-{[(1S)-1-{[(1S)-4-(carbamoylamino)-1-[(4-{[({[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)oxy]methyl}phenyl)carbamoyl]butyl]carbamoyl}-2-methylpropyl]carbamoyl}butanoic acid (L2-PIII-1)

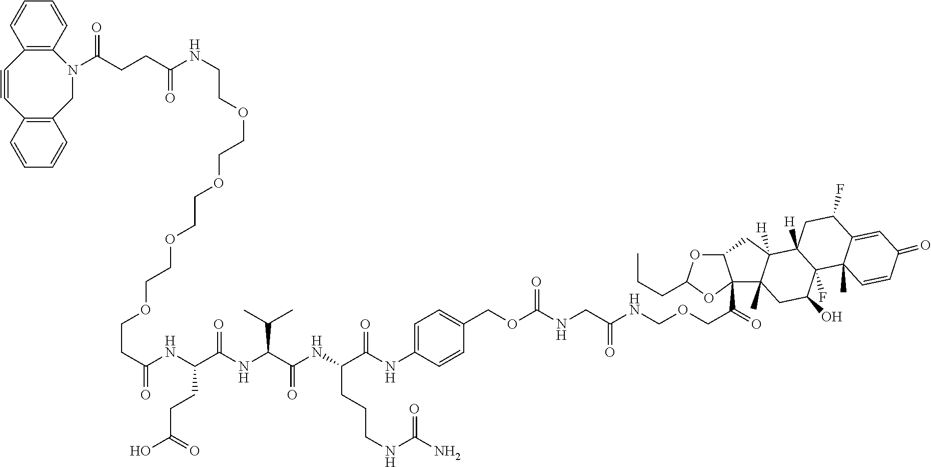 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)OCc2ccc(NC(=O)[C@H](CCCNC(N)=O)NC(=O)[C@@H](NC(=O)[C@H](CCC(=O)O)NC(=O)CCOCCOCCOCCOCCNC(=O)CCC(=O)N3Cc4ccccc4C#Cc4ccccc43)C(C)C)cc2)O1]

To a solution of compound L-10b (9 mg, 5.5 μmol) in dioxane (0.6 mL) and water (0.2 mL) was added lithium hydroxide (0.42 mg, 16.7 μmol). The reaction mixture was stirred at RT for 4 hours, and monitored by LCMS. The volatiles were removed in vacuo and the residue was purified by prep-HPLC to give linker-payload L2-PIII-1 (3 mg, 32% yield) as a white solid. ESI m/z: 812 (M/2+1)⁺. ¹H NMR (400 MHz, DMSO_{d6}) δ 10.20 (s, 1H), 8.70-8.60 (m, 1H), 8.55-8.50 (m, 1H), 8.20-8.10 (m, 1H), 7.90-7.20 (m, 13H), 6.15-6.10 (m, 1H), 6.05 (s, 1H), 5.80-5.50 (m, 4H), 5.10-4.95 (m, 3H), 4.80 (s, 1H), 4.75-4.50 (m, 4H), 4.40-4.10 (m, 5H), 3.60-3.55 (m, 26H), 3.50-3.25 (m, 6H), 3.10-2.80 (m, 4H), 2.60-2.50 (m, 3H), 2.40-2.20 (m, 5H), 2.05-1.95 (m, 3H), 1.90-1.20 (m, 15H), 0.90-0.80 (m, 8H) ppm.

### Synthesis of Linker-Payloads L4-PI, L9-PII-1, L10-PII-1, L11-PII-2, L12-PII-2, L13-PII-1, L14-PII-1, L3-PIII-1, L9-PIII-1, L12-PIII-5, L13-PIII-1, L4-PIII-1, and L14-PIV

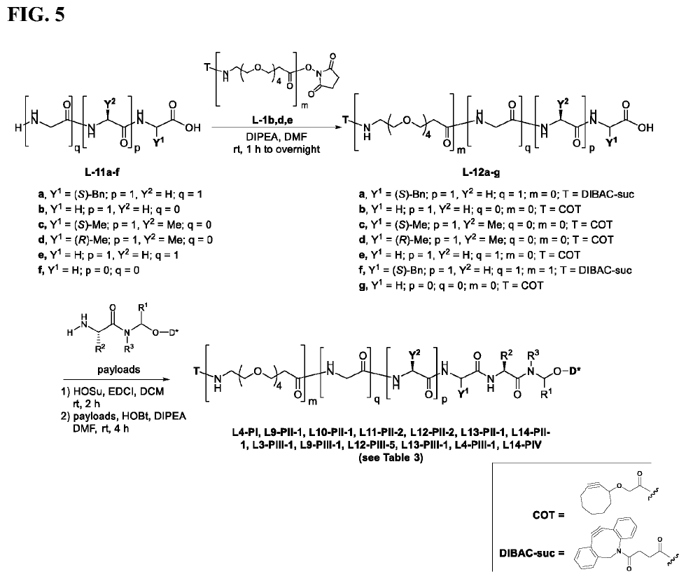

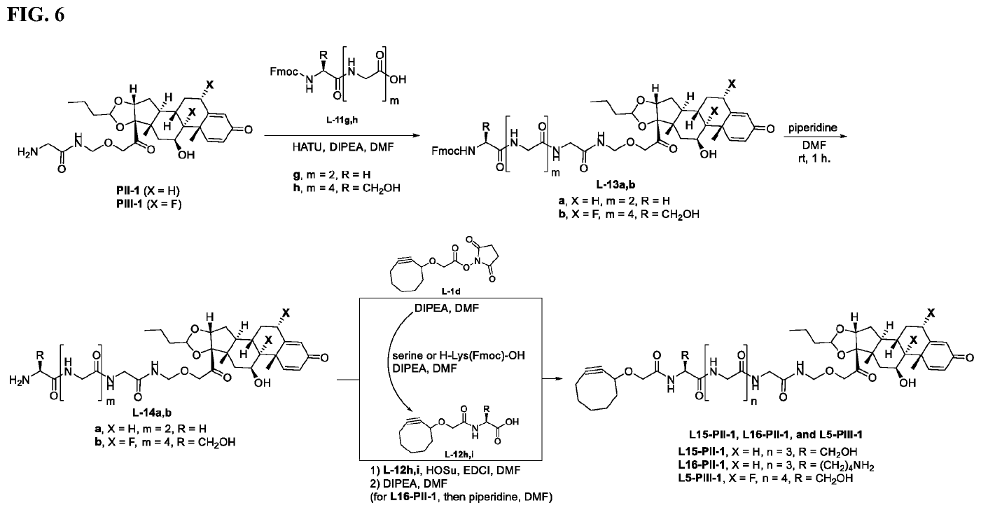

Linker-payloads L4-PI, L9-PII-1, L10-PII-1, L11-PII-2, L12-PII-2, L13-PII-1, L14-PII-1, L3-PIII-1, L9-PIII-1, L12-PIII-5, L13-PIII-1, L4-PIII-1, and L14-PIV were synthesized according to FIG. 5 and the following procedures.

### Synthesis of Intermediates L-12a-g

### (2S)-2-{2-[2-(4-{2-Azatricyclo[10.4.0.0^{4,9}]hexadeca-1(12),4(9),5,7,13,15-hexaen-10-yn-2-yl}-4-oxobutanamido)acetamido]acetamido}-3-phenylpropanoic acid (L-12a)

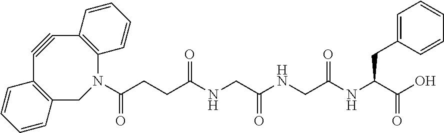 [SMILES: O=C(CCC(=O)N1Cc2ccccc2C#Cc2ccccc21)NCC(=O)NCC(=O)N[C@@H](Cc1ccccc1)C(=O)O]

To a solution of compound L-1e (0.28 g, 0.69 mmol) and peptide L-11a (Gly-Gly-Phe-OH, 0.19 g, 0.69 mmol) in DMF (10 mL) was added DIPEA (0.37 mL, 2.1 mmol), and the reaction mixture was stirred at room temperature for an hour, which was monitored by LCMS. The resulting mixture was directly purified by reversed phase flash chromatography (0-100% acetonitrile in aq. ammonium bicarbonate (10 mM)) to give compound L-12a (0.31 g, 78% yield) as a white solid. ESI m/z: 567.0 (M+H)⁺.

### 2-{2-[2-(Cyclooct-2-yn-1-yloxy)acetamido]acetamido}acetic acid (L-12b)

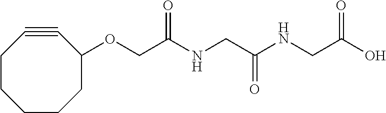 [SMILES: O=C(O)CNC(=O)CNC(=O)COC1C#CCCCCC1]

Following a similar procedure as L-12a, except substituting Gly-Gly-OH (L-11b) for Gly-Gly-Phe-OH and substituting L-1d for L-1e, intermediate L-12b (36 mg, 60% yield) was obtained as a white solid. ESI m/z: 297.2 (M+1)⁺.

### (2S)-2-[(2S)-2-[2-(Cyclooct-2-yn-1-yloxy)acetamido]propanamido]propanoic acid (L-12c)

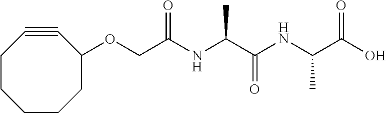 [SMILES: C[C@H](NC(=O)[C@H](C)NC(=O)COC1C#CCCCCC1)C(=O)O]

Following a similar procedure as L-12a, except substituting Ala-Ala-OH (L-11c) for Gly-Gly-Phe-OH and substituting L-1d for L-1e, intermediate L-12c (35 mg, 91% yield) was obtained as a white solid. ESI m/z: 325.3 (M+1)⁺.

### (2R)-2-[(2S)-2-[2-(Cyclooct-2-yn-1-yloxy)acetamido]propanamido]propanoic acid (L-12d)

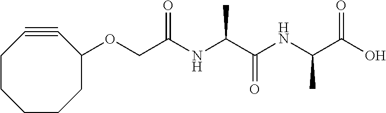 [SMILES: C[C@H](NC(=O)COC1C#CCCCCC1)C(=O)N[C@H](C)C(=O)O]

Following a similar procedure as L-12a, except substituting Ala-(D)-Ala-OH (L-11d) for Gly-Gly-Phe-OH and substituting L-1d for L-1e, intermediate L-12d (38 mg, 64% yield) was obtained as a white solid. ESI m/z: 325.2 (M+1)⁺.

### 2-(2-{2-[2-(Cyclooct-2-yn-1-yloxy)acetamido]acetamido}acetamido)acetic acid (L-12e)

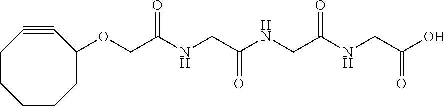 [SMILES: O=C(O)CNC(=O)CNC(=O)CNC(=O)COC1C#CCCCCC1]

To a suspension of peptide L-11e (Gly-Gly-Gly-OH, 0.34 g, 1.8 mmol) in DMF (13 mL) were added a solution of L-1d (0.50 g, 1.8 mmol) in THE (6 mL) and DIPEA (0.69 g, 5.4 mmol), and the turbid mixture was stirred at room temperature for 20 hours. The mixture was filtered, the clear filtrate solution was concentrated in vacuo, and the residue was purified by reversed phase flash chromatography (0-20% acetonitrile in water) to give compound L-12e (0.13 g, 21% yield) as a white solid. ESI m/z: 354.2 (M+H)⁺. ¹H NMR (400 MHz, DMSO_{d6}) δ 12.6 (s, 1H), 8.20 (t, J=5.6 Hz, 1H), 8.15 (t, J=6.0 Hz, 1H), 7.82 (t, J=5.6 Hz, 1H), 4.35-4.31 (m, 1H), 3.94 (d, J=14.8 Hz, 1H), 3.83-3.73 (m, 7H), 2.29-2.06 (m, 3H), 1.99-1.93 (m, 1H), 1.91-1.71 (m, 3H), 1.63-1.56 (m, 2H), 1.46-1.37 (m, 1H) ppm.

### (2S)-2-(2-{2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]acetamido}acetamido)-3-phenylpropanoic acid (L-12f)

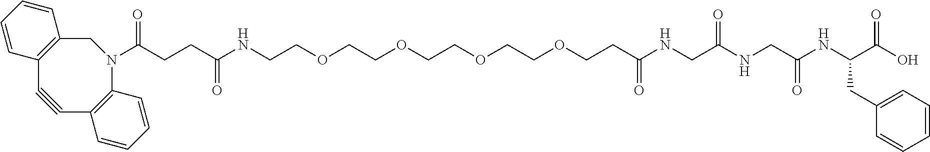 [SMILES: O=C(CCC(=O)N1Cc2ccccc2C#Cc2ccccc21)NCCOCCOCCOCCOCCC(=O)NCC(=O)NCC(=O)N[C@@H](Cc1ccccc1)C(=O)O]

Following the similar procedure as L-12a, except substituting compound L-1b for L-1e, intermediate L-12f (15 mg, 51% yield) was obtained as a white solid. ESI m/z: 408.2 (M/2+1)⁺.

### 2-[2-(Cyclooct-2-yn-1-yloxy)acetamido]acetic acid (L-12g)

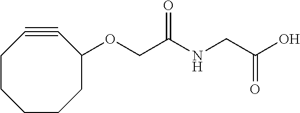 [SMILES: O=C(O)CNC(=O)COC1C#CCCCCC1]

Following a similar procedure as L-12a, except substituting glycine for Gly-Gly-Phe-OH and substituting L-1d for L-1e, intermediate L-12g (0.10 g, 61% yield) was obtained as colorless oil. ESI m/z: 240.2 (M+1)⁺.

### (2S)-2-[2-(Cyclooct-2-yn-1-yloxy)acetamido]-3-hydroxypropanoic acid (L-12h)

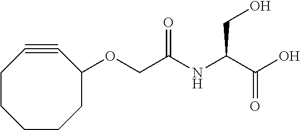 [SMILES: O=C(COC1C#CCCCCC1)N[C@@H](CO)C(=O)O]

Following a similar procedure as L-12a, except substituting serine for Gly-Gly-Phe-OH and substituting L-1d for L-1e, intermediate L-12h (90 mg, 93% yield) was obtained as yellow oil.

ESI m/z: 270.3 (M+1)⁺.

### (2S)-2-[2-(Cyclooct-2-yn-1-yloxy)acetamido]-6-{[(9H-fluoren-9-ylmethoxy)carbonyl]amino}hexanoic acid (L-12i)

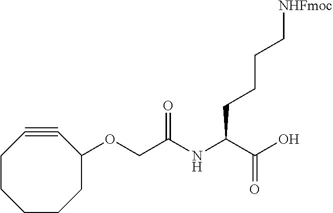

Following a similar procedure as L-12a, except substituting H-Lys(Fmoc)-OH for Gly-Gly-Phe-OH and substituting L-1d for L-1e, intermediate L-12i (30 mg, 78% yield) was obtained as a white solid. ESI m/z: 533.1 (M+1)⁺.

### General Procedure for Peptide Linker-Payloads (FIG. 5)

To a mixture of intermediate L-12 (1.0 equiv.) in DMF or DCM (25 mM) were added HATU, EDCI, or HOSu (1.5 equiv.) and DIPEA (3.0 equiv.), and the reaction mixture was stirred for 2 h at room temperature before the addition of the corresponding payload (1.0 equiv.), HOBt, and DIPEA in DMF. The reaction mixture was stirred at RT for 2-20 hours, and was monitored by LCMS. The reaction mixture was directly purified by prep-HPLC to give peptide linker-payload (22-56% yield) as a white solid.

### 4-{2-Azatricyclo[10.4.0.0^{4,9}]hexadeca-1(12),4(9),5,7,13,15-hexaen-10-yn-2-yl}-N-{[({[(1S)-1-({[({2-[(1R,2S,10S,11S,13R,14R,15S,17S)-1-fluoro-14,17-dihydroxy-2,13,15-trimethyl-5-oxotetracyclo[8.7.0.0^{2,7}.0^{11,15}]heptadeca-3,6-dien-14-yl]-2-oxoethoxy}methyl)carbamoyl]methyl}carbamoyl)-2-phenylethyl]carbamoyl}methyl)carbamoyl]methyl}-4-oxobutanamide (L4-PI)

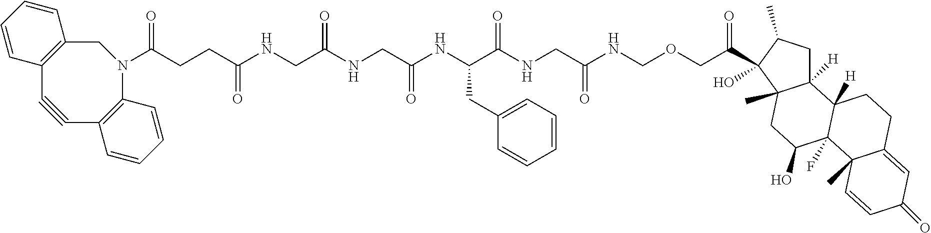 [SMILES: C[C@@H]1C[C@H]2[C@@H]3CCC4=CC(=O)C=C[C@]4(C)[C@@]3(F)[C@@H](O)C[C@]2(C)[C@@]1(O)C(=O)COCNC(=O)CNC(=O)[C@H](Cc1ccccc1)NC(=O)CNC(=O)CNC(=O)CCC(=O)N1Cc2ccccc2C#Cc2ccccc21]

Following the General Procedure for peptide linker-payloads, from L-12a and PI, linker-payload L4-PI (13 mg, 30% yield) was obtained as a white solid. ESI m/z: 606.0 (M-M_{NHCH2DEX})⁺, 635.1 (M-M_{Dex}+H)⁺. ¹H NMR (400 MHz, DMSO_{d6}) δ 8.60-8.55 (m, 1H), 8.35-8.27 (m, 1H), 8.20-7.95 (m, 3H), 7.71-7.63 (m, 1H), 7.59 (dd, J=6.7, 1.8 Hz, 1H), 7.52-7.47 (m, 1H), 7.47-7.43 (m, 1H), 7.39-7.31 (m, 2H), 7.30-7.28 (m, 1H), 7.26-7.22 (m, 3H), 7.19-7.15 (m, 1H), 6.24-6.16 (m, 1H), 6.01 (s, 1H), 5.28-5.26 (m, 1H), 5.01 (d, J=13.2 Hz, 2H), 4.60-4.53 (m, 2H), 4.52-4.44 (m, 1H), 4.21-4.10 (m, 2H), 3.78-3.66 (m, 3H), 3.64-3.57 (m, 3H), 3.10-3.00 (m, 2H), 2.96-2.87 (m, 2H), 2.83-2.75 (m, 2H), 2.69-2.58 (m, 3H), 2.37-2.23 (m, 3H), 2.17-2.03 (m, 3H), 1.84-1.73 (m, 2H), 1.66-1.55 (m, 1H), 1.53-1.45 (m, 3H), 1.38-1.30 (m, 1H), 1.27-1.22 (m, 1H), 1.16-1.01 (m, 2H), 0.87 (s, 3H), 0.78 (d, J=7.2 Hz, 3H) ppm. ¹⁹F NMR (376 MHz, DMSO_{d6}) 8-164.47 ppm.

### 2-(Cyclooct-2-yn-1-yloxy)-N-[({[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)methyl]acetamide (L9-PII-1)

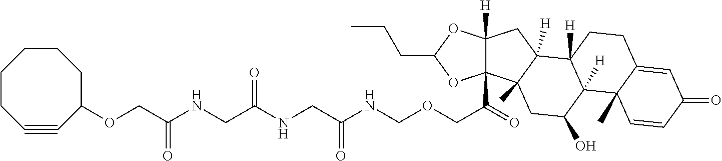

Following the General Procedure for peptide linker-payloads, from L-12g and PII-1, linker-payload L9-PII-1 (20 mg, 56% yield) was obtained as a white solid after purification by prep-HPLC (5-95% acetonitrile in aq. formic acid (0.01%)). ESI m/z: 308.1 (M-M_{bud}+H)⁺, 738.3 (M+H)⁺. 1H NMR (500 MHz, DMSO_{d6}) δ 8.68-8.64 (m, 1H), 8.29-8.26 (m, 1H), 7.85 (s, 1H), 7.34-7.31 (m, 1H), 6.16 (d, J=10.0 Hz, 1H), 5.92 (s, 1H), 5.18-5.02 (m, 1H), 4.72-4.45 (m, 5H), 4.33-4.14 (m, 3H), 3.96-3.72 (m, 6H), 2.31-1.71 (m, 13H), 1.62-1.49 (m, 6H), 1.43-1.25 (m, 7H), 1.01-0.92 (m, 2H), 0.87-0.81 (m, 6H) ppm.

### 2-(Cyclooct-2-yn-1-yloxy)-N-({[({[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)methyl]carbamoyl}methyl)acetamide (L10-PII-1)

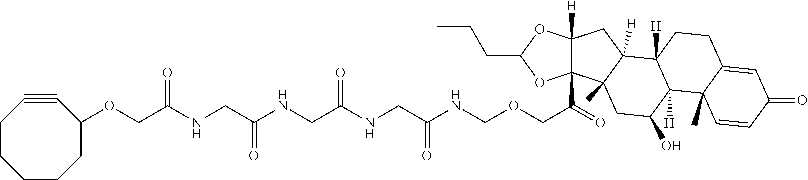 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CNC(=O)CNC(=O)COC2C#CCCCCC2)O1]

Following the General Procedure for peptide linker-payloads, from L-12b and PII-1, linker-payload L10-PII-1 (20 mg, 54% yield) was obtained as a white solid after purification by prep-HPLC (5-95% acetonitrile in aq. formic acid (0.01%)). ESI m/z: 365.1 (M-M_{bud}+H)⁺, 795.3 (M+H)⁺. 1H NMR (500 MHz, DMSO_{d6}) δ 8.69-8.63 (m, 1H), 8.24-8.19 (m, 2H), 7.86-7.83 (m, 1H), 7.33-7.31 (m, 1H), 6.17 (d, J=10.5 Hz, 1H), 5.92 (s, 1H), 5.18-5.03 (m, 1H), 4.75-4.46 (m, 5H), 4.35-4.15 (m, 3H), 3.95-3.71 (m, 8H), 2.31-1.70 (m, 13H), 1.65-1.53 (m, 6H), 1.48-1.24 (m, 7H), 1.04-0.81 (m, 8H) ppm.

### (2S)-2-[(2S)-2-[(2S)-2-[2-(Cyclooct-2-yn-1-yloxy)acetamido]propanamido]propanamido]-N-({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)propanamide (L11-PII-2)

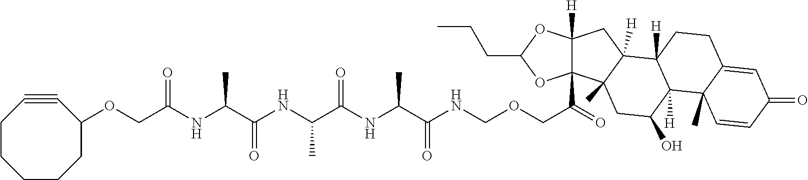 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)[C@H](C)NC(=O)[C@H](C)NC(=O)[C@H](C)NC(=O)COC2C#CCCCCC2)O1]

Following the General Procedure for peptide linker-payloads, from L-12c and PII-2, linker-payload L11-PII-2 (3.0 mg, 22% yield) was obtained as a white solid after purification by prep-HPLC (5-95% acetonitrile in aq. ammonium bicarbonate (10 mM)). ESI m/z: 407.3 (M-M_{bud}+H)⁺, 859.5 (M+Na)⁺. ¹H NMR (400 MHz, methanol_{d4}) δ 7.47 (dd, J=10.4, 3.6 Hz, 1H), 6.29-6.26 (m, 1H), 6.03 (s, 1H), 5.36 (t, J=4.4 Hz, 1H), 5.22-5.14 (m, 1H), 4.80-4.73 (m, 2H), 4.65-4.48 (m, 2H), 4.46-4.25 (m, 6H), 4.10-3.90 (m, 2H), 2.71-2.63 (m, 1H), 2.42-2.37 (m, 1H), 2.30-2.03 (m, 7H), 1.98-1.80 (m, 5H), 1.73-1.60 (m, 6H), 1.51 (s, 3H), 1.44-1.38 (m, 10H), 1.24-1.23 (m, 2H), 0.97-0.90 (m, 7H) ppm.

### (2S)-2-[(2R)-2-[(2S)-2-[2-(Cyclooct-2-yn-1-yloxy)acetamido]propanamido]propanamido]-N-({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)propanamide (L12-PII-2)

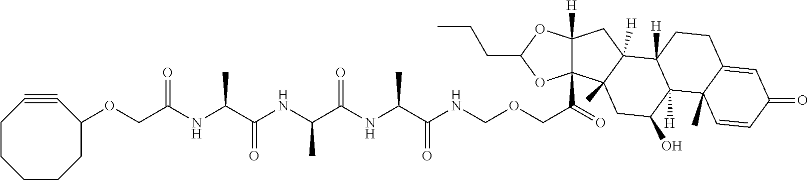 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)[C@H](C)NC(=O)[C@@H](C)NC(=O)[C@H](C)NC(=O)COC2C#CCCCCC2)O1]

Following the General Procedure for peptide linker-payloads, from L-12d and PII-2, linker-payload L12-PII-2 (3.5 mg, 22% yield) was obtained as a white solid after purification by prep-HPLC (5-95% acetonitrile in aq. ammonium bicarbonate (10 mM)). ESI m/z: 407.3 (M-M_{bud}+H)⁺, 859.5 (M+Na)⁺. ¹H NMR (400 MHz, methanol_{d4}) δ 7.47 (dd, J=10.4, 3.6 Hz, 1H), 6.30-6.25 (m, 1H), 6.03 (s, 1H), 5.36 (t, J=4.4 Hz, 1H), 5.23-5.12 (m, 1H), 4.84-4.70 (m, 2H), 4.66-4.48 (m, 2H), 4.45-4.24 (m, 6H), 4.06-3.89 (m, 2H), 2.72-2.61 (m, 1H), 2.43-2.35 (m, 1H), 2.30-2.01 (m, 7H), 1.98-1.80 (m, 5H), 1.75-1.58 (m, 6H), 1.51 (s, 3H), 1.44-1.38 (m, 10H), 1.36-1.27 (m, 2H), 0.98-0.88 (m, 7H) ppm.

### 2-(Cyclooct-2-yn-1-yloxy)-N-{[({[({[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)methyl]carbamoyl}methyl)carbamoyl]methyl}acetamide (L13-PII-1)

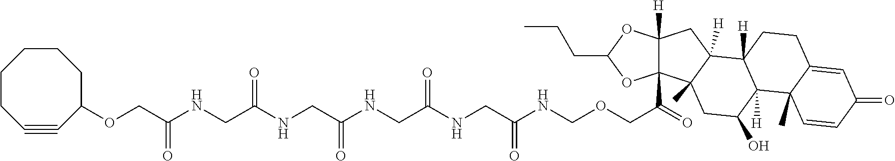 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CNC(=O)CNC(=O)CNC(=O)COC2C#CCCCCC2)O1]

Following the General Procedure for peptide linker-payloads, from L-12e and PII-1, linker-payload L13-PII-1 (14 mg, 16% yield) was obtained as a white solid after purification by prep-HPLC (5-95% acetonitrile in aq. ammonia (10 mM)). ESI m/z: 422.3 (M-M_{bud}+H)⁺, 874.5 (M+Na)⁺. ¹H NMR (500 MHz, DMSO_{d6}) δ 8.68-8.63 (m, 1H), 8.22-8.14 (m, 3H), 7.84-7.82 (m, 1H), 7.33-7.30 (m, 1H), 6.18-6.16 (m, 1H), 5.92 (s, 1H), 5.18-5.16 (m, 0.5H), 5.04-5.02 (m, 0.5H), 4.73-4.45 (m, 5H), 4.33-4.14 (m, 3H), 3.95-3.71 (m, 10H), 2.36-1.69 (m, 12H), 1.62-1.50 (m, 6H), 1.44-1.23 (m, 8H), 1.01-0.81 (m, 8H) ppm.

### 4-{2-Azatricyclo[10.4.0.0^{4,9}]hexadeca-1(12),4(9),5,7,13,15-hexaen-10-yn-2-yl}-N-{[({[(1S)-1-({[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)-2-phenylethyl]carbamoyl}methyl)carbamoyl]methyl}-4-oxobutanamide (L14-PII-1)

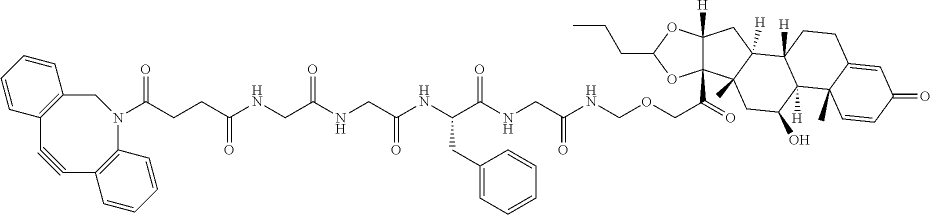 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)[C@H](Cc2ccccc2)NC(=O)CNC(=O)CNC(=O)CCC(=O)N2Cc3ccccc3C#Cc3ccccc32)O1]

Following the General Procedure for peptide linker-payloads, from L-12a and PII-1, linker-payload L14-PII-1 (0.22 g, 64% yield) was obtained as a white solid after purification by prep-HPLC (5-95% acetonitrile in aq. ammonium bicarbonate (10 mM)). ESI m/z: 606.0 (M-M_{NHCH2Bud})⁺, 635.0 (M-M_{Bud}+H)⁺. ¹H NMR (400 MHz, DMSO_{d6}) δ 8.63-8.53 (m, 1H), 8.33-8.28 (m, 1H), 8.20-7.97 (m, 3H), 7.70-7.63 (m, 1H), 7.61-7.57 (m, 1H), 7.51-7.43 (m, 3H), 7.39-7.33 (m, 2H), 7.32-7.30 (m, 1H), 7.29-7.26 (m, 1H), 7.25-7.23 (m, 4H), 7.20-7.16 (m, 1H), 6.19-6.11 (m, 1H), 5.92 (s, 1H), 5.16 (t, J=4.8 Hz, 0.5H), 5.03 (d, J=5.2 Hz, 1H), 4.99 (s, 0.5H), 4.73-4.72 (m, 2H), 4.64-4.56 (m, 3H), 4.51-4.46 (m, 2H), 4.29 (s, 1H), 4.19 (t, J=18.5 Hz, 1H), 3.79-3.68 (m, 3H), 3.62-3.57 (m, 3H), 3.57-3.52 (m, 1H), 3.07 (dd, J=13.7, 4.0 Hz, 1H), 2.83-2.77 (m, 1H), 2.71-2.60 (m, 1H), 2.36-2.24 (m, 2H), 2.13-2.04 (m, 2H), 2.02-1.93 (m, 1H), 1.84-1.81 (m, 0.5H), 1.80-1.73 (m, 2H), 1.72-1.68 (m, 0.5H), 1.63-1.57 (m, 1H), 1.57-1.47 (m, 3H), 1.44-1.39 (m, 1H), 1.38-1.37 (m, 3H), 1.34-1.23 (m, 2H), 1.01-0.89 (m, 2H), 0.88-0.80 (m, 6H) ppm.

### 1-(4-{2-Azatricyclo[10.4.0.0^{4,9}]hexadeca-1(12),4(9),5,7,13,15-hexaen-10-yn-2-yl}-4-oxobutanamido)-N-{[({[(1S)-1-({[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)-2-phenylethyl]carbamoyl}methyl)carbamoyl]methyl}-3,6,9,12-tetraoxapentadecan-15-amide

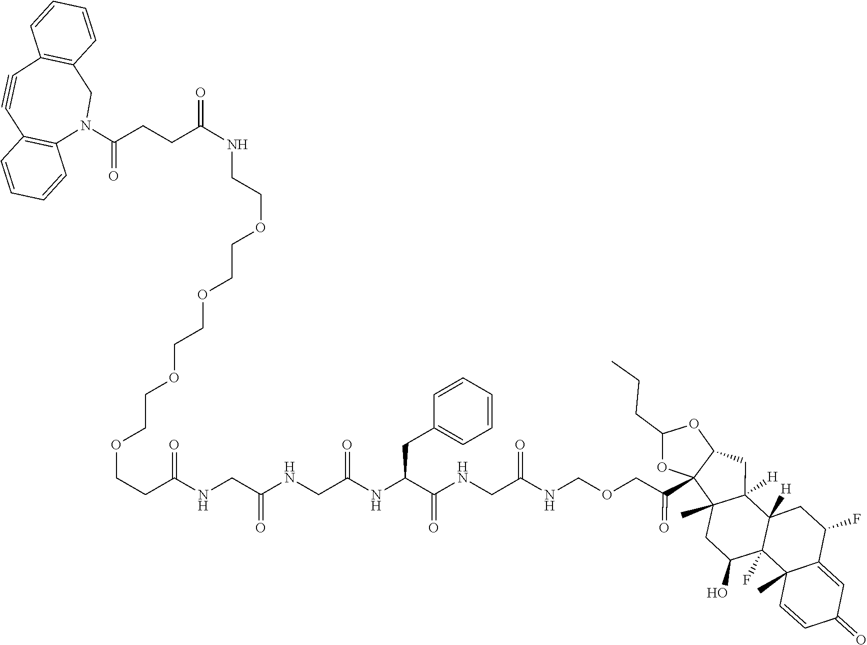 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)[C@H](Cc2ccccc2)NC(=O)CNC(=O)CNC(=O)CCOCCOCCOCCOCCNC(=O)CCC(=O)N2Cc3ccccc3C#Cc3ccccc32)O1]

To a solution of compound L-12f (60 mg, 74 μmol) in DCM (6 mL) were added HOSu (19 mg, 0.16 mmol), and EDCI (31 mg, 0.16 mmol), and the mixture was stirred at RT for an hour. The reaction mixture was diluted with DCM (40 mL) and washed with water (20 mL) and brine (20 mL). The organic solution was dried over anhydrous sodium sulfate and concentrated in vacuo. The residue was then dissolved in dry DMF (2 mL). To the solution were added DIPEA (45 mg, 0.35 mmol) and PIII-1 (48 mg, 88 μmol), and the mixture was stirred at RT for 2 hours, which was monitored by LCMS. The reaction mixture was directly purified by prep-HPLC to give L3-PIII-1 (5 mg, 4% yield) as a white solid. ESI m/z: 882.3 (M-Bud)⁺. ¹H NMR (400 MHz, DMSO_{d6}) δ 8.65-8.56 (m, 1H), 8.32-8.30 (m, 1H), 8.20-7.97 (m, 3H), 7.79-7.72 (m, 1H), 7.71-7.66 (m, 1H), 7.65-7.61 (m, 1H), 7.53-7.46 (m, 3H), 7.42-7.28 (m, 3H), 7.27-7.20 (m, 5H), 7.19-7.14 (m, 1H), 6.31-6.25 (m, 1H), 6.10 (s, 1H), 5.72-5.48 (m, 2H), 5.49-5.48 (m, 1H), 5.09-4.98 (m, 1H), 4.68-4.48 (m, 4H), 4.30-4.17 (m, 2H), 3.78-3.65 (m, 5H), 3.64-3.56 (m, 4H), 3.54-3.39 (m, 12H), 3.31-3.27 (m, 2H), 3.11-3.01 (m, 3H), 2.84-2.77 (m, 1H), 2.71-2.56 (m, 2H), 2.42-2.36 (m, 2H), 2.29-2.17 (m, 2H), 2.08-1.95 (m, 3H), 1.83-1.67 (m, 2H), 1.62-1.24 (m, 10H), 0.91-0.75 (m, 6H) ppm.

### 2-(Cyclooct-2-yn-1-yloxy)-N-[({[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)methyl]acetamide (L9-PIII-1)

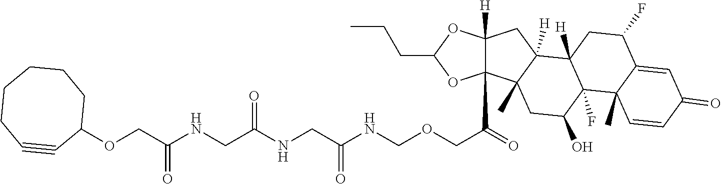 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)CNC(=O)COC2C#CCCCCC2)O1]

Following the General Procedure for peptide linker-payloads, from L-12g and PIII-1, linker-payload L9-PIII-1 (13 mg, 31% yield) was obtained as a white solid after purification by prep-HPLC (5-95% acetonitrile in aq. ammonium bicarbonate (10 mM)). ESI m/z: 774.3 (M+H)⁺, 308.1 (M-M111+H)⁺. ¹H NMR (400 MHz, DMSO_{d6}) δ 8.66 (t, J=6.7 Hz, 1H), 8.27 (t, J=5.8 Hz, 1H), 7.86 (t, J=5.6 Hz, 1H), 7.27 (dd, J=10.3, 0.8 Hz, 1H), 6.30 (dd, J=10.2, 1.8 Hz, 1H), 6.11 (s, 1H), 5.73-5.53 (m, 1H), 5.47 (d, J=2.6 Hz, 1H), 4.78-4.73 (m, 1H), 4.65 (t, J=4.1 Hz, 1H), 4.62-4.56 (m, 2H), 4.53-4.45 (m, 1H), 4.35-4.29 (m, 1H), 4.27-4.16 (m, 2H), 3.97-3.69 (m, 6H), 2.69-2.54 (m, 1H), 2.30-2.14 (m, 3H), 2.13-1.81 (m, 5H), 1.80-1.68 (m, 3H), 1.61-1.51 (m, 5H), 1.49 (s, 3H), 1.45-1.29 (m, 4H), 1.24 (s, 1H), 0.86 (t, J=7.4 Hz, 3H), 0.81 (s, 3H) ppm.

### (2S)-2-[(2R)-2-[(2S)-2-[2-(Cyclooct-2-yn-1-yloxy)acetamido]propanamido]propanamido]-N-({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)propanamide (L12-PIII-5)

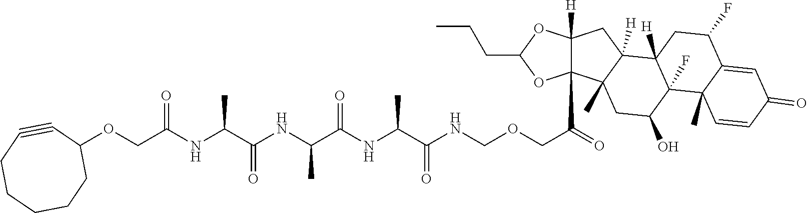

Following the General Procedure for peptide linker-payloads, from L-12d and PIII-5, linker-payload L12-PIII-5 (3.5 mg, 21% yield) was obtained as a white solid after purification by prep-HPLC (5-95% acetonitrile in aq. ammonium bicarbonate (10 mM)). ESI m/z: 895.5 (M+Na)⁺, 407.3 (M-M_{III}+H)⁺. H NMR (400 MHz, methanol_{d4}) δ 7.40-7.32 (m, 1H), 6.37-6.34 (m, 1H), 6.32 (s, 1H), 5.64-5.47 (m, 1H), 5.37-5.35 (m, 1H), 4.80-4.73 (m, 2H), 4.69-4.66 (m, 1H), 4.57-4.50 (m, 1H), 4.41-4.25 (m, 6H), 4.10-3.90 (m, 2H), 2.74-2.57 (m, 1H), 2.37-2.29 (m, 1H), 2.26-2.21 (m, 5H), 2.09-2.01 (m, 2H), 1.98-1.76 (m, 4H), 1.72-1.60 (m, 6H), 1.59 (s, 3H), 1.46-1.38 (m, 10H), 1.35-1.33 (m, 1H), 0.97-0.90 (m, 7H) ppm.

### 2-(Cyclooct-2-yn-1-yloxy)-N-{[({[({[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)methyl]carbamoyl}methyl)carbamoyl]methyl}acetamide (L13-PIII-1)

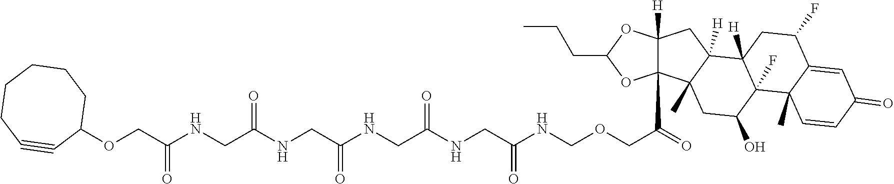

Following the General Procedure for peptide linker-payloads, from L-12e and PIII-1, linker-payload L13-PIII-1 (8 mg, 17% yield) was obtained as a white solid after purification by prep-HPLC (5-95% acetonitrile in aq. ammonium bicarbonate (10 mM)). ESI m/z: 888.4 (M+H)⁺, 422.1 (M-M111+H)⁺. ¹H NMR (400 MHz, DMSO_{d6}) δ 8.73-8.63 (m, 1H), 8.24-8.17 (m, 2H), 7.85 (t, J=5.2 Hz, 1H), 7.27 (d, J=9.6 Hz, 1H), 6.29 (dd, J=10.4, 1.6 Hz, 1H), 6.11 (s, 1H), 5.74-5.47 (m, 2H), 4.77-4.73 (m, 1H), 4.66-4.54 (m, 3H), 4.53-4.44 (m, 1H), 4.35-4.29 (m, 1H), 4.28-4.15 (m, 2H), 3.97-3.90 (m, 1H), 3.84-3.69 (m, 8H), 3.30 (s, 1H), 2.69-2.54 (m, 1H), 2.35-2.14 (m, 3H), 2.12-1.91 (m, 4H), 1.90-1.66 (m, 4H), 1.61-1.51 (m, 5H), 1.49 (s, 3H), 1.45-1.20 (m, 5H), 0.96-0.92 (m, 1H), 0.89-0.79 (m, 6H) ppm.

### 4-{2-Azatricyclo[10.4.0.0^{4,9}]hexadeca-1(12),4(9),5,7,13,15-hexaen-10-yn-2-yl}-N-{[({[(1S)-1-({[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)-2-phenylethyl]carbamoyl}methyl)carbamoyl]methyl}-4-oxobutanamide (L4-PIII-1)

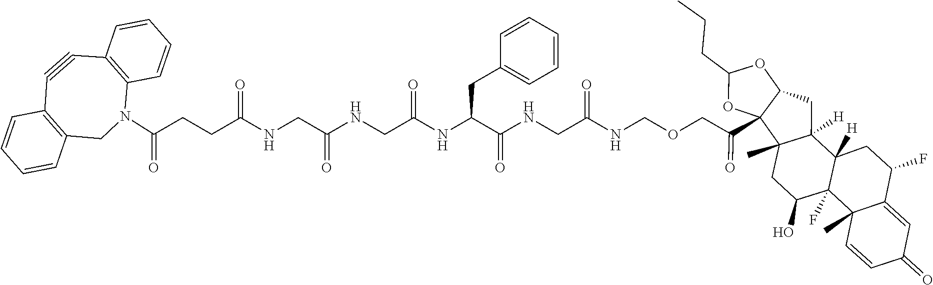 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)[C@H](Cc2ccccc2)NC(=O)CNC(=O)CNC(=O)CCC(=O)N2Cc3ccccc3C#Cc3ccccc32)O1]

Following the General Procedure for peptide linker-payloads, from L-12a and PIII-1, linker-payload L4-PIII-1 (23 mg, 25% yield) was obtained as a white solid after purification by prep-HPLC (5-95% acetonitrile in aq. ammonium bicarbonate (10 mM)). ESI m/z: 1124.5 (M+Na)⁺. ¹H NMR (400 MHz, DMSO_{d6}) δ 8.65-8.56 (m, 1H), 8.32-8.30 (m, 1H), 7.97-8.20 (m, 3H), 7.72-7.62 (m, 1H), 7.60-7.56 (m, 1H), 7.52-7.43 (m, 3H), 7.40-7.28 (m, 4H), 7.27-7.20 (m, 5H), 7.19-7.14 (m, 1H), 6.31-6.25 (m, 1H), 6.10 (s, 1H), 5.72-5.52 (m, 1H), 5.49-5.48 (m, 1H), 5.05-4.98 (m, 1H), 4.68-4.48 (m, 4H), 4.74 (s, 1H), 4.62-4.44 (m, 4H), 4.30-4.17 (m, 2H), 3.78-3.65 (m, 3H), 3.05-3.01 (m, 1H), 2.83-2.72 (m, 1H), 2.71-2.56 (m, 2H), 2.34-2.24 (m, 2H), 2.12-1.95 (m, 3H), 1.83-1.67 (m, 2H), 1.65-1.51 (m, 4H), 1.47 (s, 3H), 1.46-1.22 (m, 3H), 0.86-0.78 (m, 6H) ppm.

### (1S,4aS,10aR)-N-[(1S,4aS,10aR)-6-[2-({2-[(2S)-2-{2-[2-(4-{2-Azatricyclo[10.4.0.0^{4,9}]hexadeca-1(12),4(9),5,7,13,15-hexaen-10-yn-2-yl}-4-oxobutanamido)acetamido]acetamido}-3-phenylpropanamido]acetamido}methoxy)acetamido]-1,4a-dimethyl-1,2,3,4,4a,9,10,10a-octahydrophenanthrene-1-carbonyl]-6-hydroxy-1,4a-dimethyl-1,2,3,4,4a,9,10,10a-octahydrophenanthrene-1-carboxamide (L14-PIV)

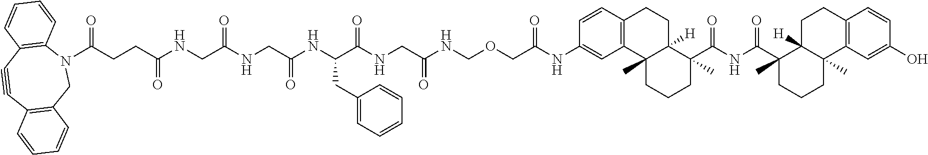 [SMILES: C[C@]1(C(=O)NC(=O)[C@@]2(C)CCC[C@]3(C)c4cc(NC(=O)COCNC(=O)CNC(=O)[C@H](Cc5ccccc5)NC(=O)CNC(=O)CNC(=O)CCC(=O)N5Cc6ccccc6C#Cc6ccccc65)ccc4CC[C@@H]23)CCC[C@]2(C)c3cc(O)ccc3CC[C@@H]12]

Following the General Procedure for peptide linker-payloads, from L-12a and PIV, linker-payload L14-PIV (8.4 mg, 46% yield) was obtained as a white solid after purification by prep-HPLC (5-95% acetonitrile in aq. ammonium bicarbonate (10 mM)). ESI m/z: 635 (M-M_{IV}+H)⁺. ¹H NMR (400 MHz, DMSO_{d6}) δ 9.53 (s, 1H), 8.99 (s, 1H), 8.70 (d, J=5.2 Hz, 1H), 8.36 (d, J=5.0 Hz, 1H), 8.20-7.96 (m, 4H), 7.69-7.62 (m, 1H), 7.61-7.56 (m, 2H), 7.51-7.39 (m, 4H), 7.38-7.32 (m, 2H), 7.31-7.20 (m, 5H), 7.19-7.13 (m, 1H), 6.95 (d, J=8.7 Hz, 1H), 6.82 (d, J=8.2 Hz, 1H), 6.63 (d, J=2.2 Hz, 1H), 6.50 (dd, J=8.2 and 2.2 Hz, 1H), 4.99 (dd, J=13.9 and 1.9 Hz, 1H), 4.64 (d, J=6.6 Hz, 2H), 4.51-4.43 (m, 1H), 4.00 (s, 2H), 3.80-3.66 (m, 3H), 3.62-3.56 (m, 3H), 3.08-2.99 (m, 1H), 2.91-2.59 (m, 7H), 2.34-2.23 (m, 3H), 2.20-2.10 (m, 4H), 2.08-2.00 (m, 1H), 1.95-1.72 (m, 5H), 1.67-1.51 (m, 4H), 1.27 (s, 3H), 1.27 (s, 3H), 1.25-1.22 (m, 2H), 1.17-1.08 (m, 2H), 0.99 (s, 3H), 0.98 (s, 3H) ppm.

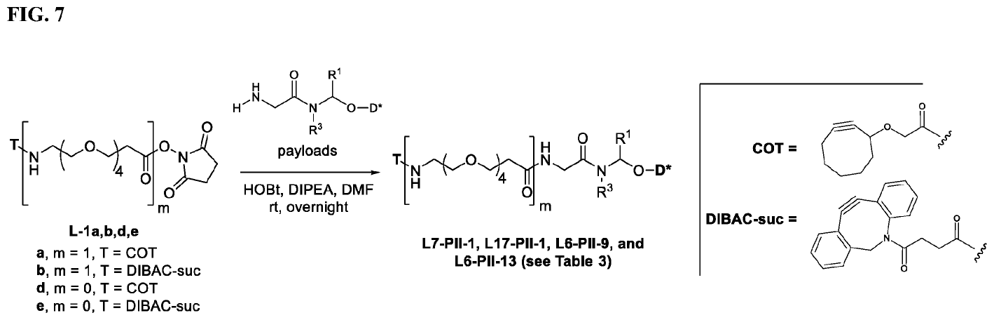

### Synthesis of Linker-Payloads L15-PII-1, L16-PII-1, and L5-PIII-1 (FIG. 6)

Linker-payload L15-PII-1, L16-PII-1, and L5-PIII-1 was synthesized according to FIG. 6 and the following procedures.

### 2-Amino-N-({[({[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)methyl]carbamoyl}methyl)acetamide (L-14a)

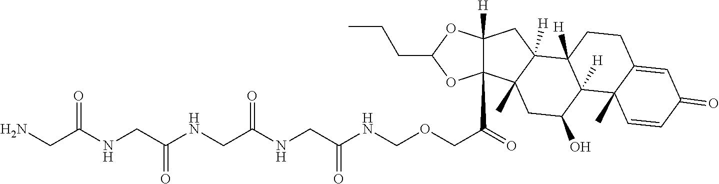

To a mixture of Fmoc-Gly-Gly-Gly-OH (L-11g, 41 mg, 0.10 mmol) in DMF (4.0 mL) were added HATU (38 mg, 0.10 mmol) and DIPEA (39 mg, 0.30 mmol), and the mixture was stirred at room temperature for 15 minutes before the addition of compound PII-1 (50 mg, 97 μmol). The resulting mixture was stirred at room temperature for an hour, which was monitored by LCMS. The reaction mixture was directly purified by reversed phase flash chromatography (0-100% acetonitrile in aq. ammonium bicarbonate (10 mM)) to give compound L-13a (36 mg, ESI m/z: 932.1 (M+Na)⁺) as a white solid, which was then dissolved into DMF (2 mL). To the solution was added piperidine (17 mg, 0.20 mmol), and the mixture was stirred at room temperature for 2 hours until Fmoc was totally removed, as monitored by LCMS. The resulting mixture was directly purified by prep-HPLC (5-95% acetonitrile in aq. ammonium bicarbonate (10 mM)) to give compound L-14a (26 mg, 39% yield) as a white solid. ESI m/z: 688.2 (M+H)⁺.

### (2S)-2-[2-(Cyclooct-2-yn-1-yloxy)acetamido]-3-hydroxy-N-{[({[({[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)methyl]carbamoyl}methyl)carbamoyl]methyl}propanamide (L15-PII-1)

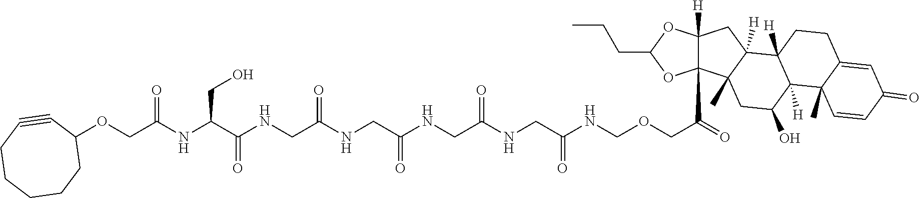 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CNC(=O)CNC(=O)CNC(=O)[C@H](CO)NC(=O)COC2C#CCCCCC2)O1]

To a solution of compound L-12h (50 mg, 0.19 mmol) in DMF (4.0 mL) were added HOSu (40 mg, 0.38 mmol) and EDCI (70 mg, 0.38 mmol), and the mixture was stirred at room temperature for 12 hours until L-12h was totally consumed. The reaction mixture was directly purified by reversed phase flash chromatography (0-100% acetonitrile in water) to give the active ester. The active ester (9.0 mg, crude) was added into a mixture of compound L-14a (15 mg, 22 μmol) and DIPEA (9.0 mg, 70 μmol) in DMF (2 mL), and the reaction mixture was stirred at room temperature for 30 minutes, which was monitored by LCMS. The resulting mixture was directly purified by prep-HPLC (5-95% acetonitrile in aq. ammonium bicarbonate (10 mM)) to give linker-payload L15-PII-1 (11 mg, 14% yield from L-14a) as a white solid. ESI m/z: 509.1 (M-M_{Bud}+H)⁺; 961.4 (M+Na)⁺. ¹H NMR (400 MHz, DMSO_{d6}) δ 8.80-8.65 (m, 1H), 8.41-8.06 (m, 3H), 7.37-7.27 (m, 1H), 6.17 (d, J=9.6 Hz, 1H), 5.92 (s, 1H), 5.20-5.01 (m, 1H), 4.84-4.43 (m, 5H), 4.37-4.26 (m, 2H), 4.25-4.14 (m, 1H), 3.97-3.80 (m, 1H), 3.79-3.55 (m, 8H), 3.24-3.16 (m, 1H), 3.02-2.90 (m, 1H), 2.43-2.38 (m, 1H), 2.34-2.27 (m, 2H), 2.24-2.05 (m, 3H), 2.03-1.93 (m, 2H), 1.86-1.67 (m, 4H), 1.63-1.47 (m, 5H), 1.45-1.20 (m, 8H), 0.98-0.90 (m, 5H), 0.89-0.78 (m, 6H) ppm.

### (2S)-6-Amino-2-[2-(cyclooct-2-yn-1-yloxy)acetamido]-N-{[({[({[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)methyl]carbamoyl}methyl)carbamoyl]methyl}hexanamide (L16-PII-1)

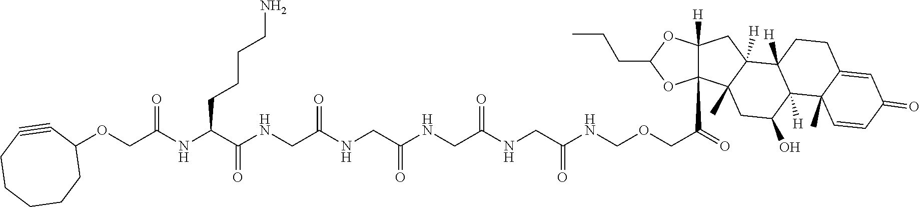 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CNC(=O)CNC(=O)CNC(=O)[C@H](CCCCN)NC(=O)COC2C#CCCCCC2)O1]

To a solution of L-12i (25 mg, 47 μmol) in DMF (2.0 ml) were added HOSu (11 mg, 94 μmol) and EDCI (18 mg, 94 μmol), and the reaction mixture was stirred at room temperature for 2 hours. The resulting mixture was directly purified by reversed phase flash chromatography (0-100% acetonitrile in water) to give the active ester (17 mg). The active ester (12 mg, 18 μmol) was added into a solution of compound L-14a (12 mg, 17 μmol) in DMF (2.0 mL) before the addition of DIPEA (6.6 mg, 51 μmol). The reaction mixture was then stirred at room temperature for an hour, which was monitored by LCMS. The resulting mixture was separated by reversed phase flash chromatography (0-100% acetonitrile in aq. ammonium bicarbonate (10 mM)) to give Fmoc-L16-PII-1 (11 mg) as a white solid, which was dissolved in DMF (2.0 mL). To the solution was added piperidine (4.3 mg, 51 μmol), and the mixture was stirred at room temperature for an hour until Fmoc was totally removed, as monitored by LCMS. The resulting mixture was directly purified by reversed phase flash chromatography (0-100% acetonitrile in aq. ammonium bicarbonate (10 mM)) to give linker-payload L16-PII-1 (12 mg, 76% yield from L-14a) as a white solid. ESI m/z: 490.9 (M/2+H)⁺, 980.7 (M+H)⁺. ¹H NMR (400 MHz, DMSO_{d6}) δ 8.80-8.73 (m, 1H), 8.49-8.41 (m, 2H), 8.33-8.28 (m, 1H), 8.27-8.21 (m, 1H), 7.74 (dd, J=14.0, 7.9 Hz, 1H), 7.32 (dd, J=10.1, 2.7 Hz, 1H), 6.19-6.16 (m, 1H), 5.93 (s, 1H), 5.18-5.16 (m, 0.5H), 5.03 (d, J=7.4 Hz, 0.5H), 4.85 (s, 1H), 4.72 (d, J=4.2 Hz, 0.5H), 4.65-4.53 (m, 3H), 4.51-4.44 (m, 1H), 4.38-4.28 (m, 3H), 4.24 (s, 0.5H), 4.19-4.15 (m, 1H), 3.95-3.92 (m, 1H), 3.85-3.81 (m, 1H), 3.76-3.71 (m, 6H), 2.76-2.68 (m, 2H), 2.35-2.25 (m, 2H), 2.25-2.15 (m, 2H), 2.15-1.83 (m, 7H), 1.82-1.64 (m, 6H), 1.64-1.46 (m, 8H), 1.45-1.23 (m, 9H), 1.17-1.06 (m, 1H), 1.02-0.91 (m, 2H), 0.90-0.75 (m, 6H) ppm.

### (2S)-2-Amino-N-[({[({[({[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)methyl]carbamoyl}methyl)carbamoyl]methyl}carbamoyl)methyl]-3-hydroxypropanamide (L-14b)

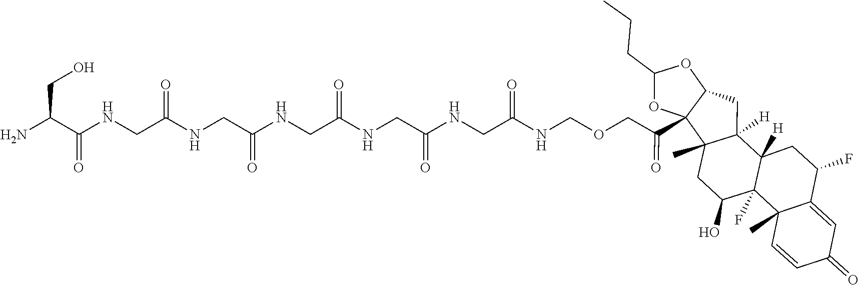 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)CNC(=O)CNC(=O)CNC(=O)CNC(=O)[C@@H](N)CO)O1]

To a solution of payload PIII-1 (0.20 g, 0.36 mmol) and peptide Fmoc-Ser-Gly-Gly-Gly-Gly L-11h (0.20 g, 0.36 mmol) in DMF (4 mL) were added HATU (0.21 g, 0.55 mmol) and DIPEA (0.14 g, 1.1 mmol), and the mixture was stirred at RT overnight, which was monitored by LCMS. The reaction mixture was purified by prep-HPLC to give L-13b (0.16 g, ESI m/z: 624 (M-M_{III}+H)⁺), which was dissolved in DMF (2 mL). To the solution was added piperidine (42 mg, 0.50 mmol) and the mixture was stirred at RT for an hour until Fmoc was totally removed according to LCMS. The reaction mixture was purified by prep-HPLC to give compound L-14b (98 mg, 77% yield) as a white solid. ESI m/z: 868 (M+1)⁺.

### 4-{2-Azatricyclo[10.4.0.0^{4,9}]hexadeca-1(12),4(9),5,7,13,15-hexaen-10-yn-2-yl}-N-[(1S)-1-{[({[({[({[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)methyl]carbamoyl}methyl)carbamoyl]methyl}carbamoyl)methyl]carbamoyl}-2-hydroxyethyl]-4-oxobutanamide (L5-PIII-1)

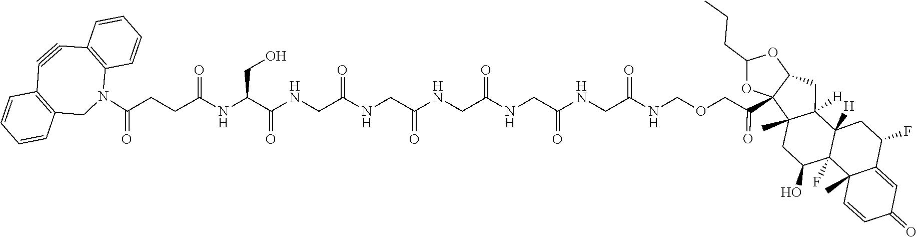 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)CNC(=O)CNC(=O)CNC(=O)CNC(=O)[C@H](CO)NC(=O)CCC(=O)N2Cc3ccccc3C#Cc3ccccc32)O1]

To a solution of compound L-14b (0.11 g, 0.13 mmol) in DMF (4 mL) were added compound L-1e (39 mg, 0.13 mmol), HATU (72 mg, 0.19 mmol) and DIPEA (49 mg, 0.38 mmol), and the mixture was stirred at RT for 3 hours, which was monitored by LCMS. The resulting mixture was directly purified by prep-HPLC to give linker-payload L5-PIII-1 (35 mg, 24% yield) as a white solid. ESI m/z: 1177 (M+23)⁺. ¹H NMR (400 MHz, DMSO_{d6}) δ 8.66 (t, J=6.4 Hz, 1H), 8.24-7.93 (m, 6H), 7.71-7.66 (m, 1H), 7.61 (t, J=6.0 Hz, 1H), 7.54-7.43 (m, 3H), 7.41-7.24 (m, 4H), 6.30 (dd, J=10 and 2.0 Hz, 1H), 6.11 (s, 1H), 5.74-5.46 (m, 2H), 5.08-4.97 (m, 1H), 4.94-4.84 (m, 1H), 4.78-4.73 (m, 1H), 4.67-4.57 (m, 3H), 4.54-4.46 (m, 1H), 4.39-4.11 (m, 3H), 3.83-3.67 (m, 9H), 3.66-3.47 (m, 3H), 2.70-2.57 (m, 1H), 2.39-2.22 (m, 2H), 2.17-1.95 (m, 3H), 1.84-1.67 (m, 2H), 1.63-1.51 (m, 4H), 1.481 (s, 3H), 1.44-1.20 (m, 4H), 0.95 (d, J=6.4 Hz, 1H), 0.86-0.78 (m, 6H) ppm.

### Synthesis of Linker-Payloads L7-PII-1, L17-PII1, L6-PII-9, and L6-PII-13

Linker-payloads L6-PII-9 and L7-PII-1 were synthesized according to FIG. 7 and the following procedures.

### General Procedure for pH-Sensitive Linker-Payloads

To a mixture of compound L1-a,b,d,e (1.0 equiv.) in DMF (25 mM) were added payload PII-1, PII-9, or PII-13 (1.0 equiv.) and DIPEA (3.0 equiv.), and then the reaction mixture was stirred at RT for 2 hours, which was monitored by LCMS. The resulting mixture was directly purified by prep-HPLC to give linker-payload L7-PII-1, L17-PII1, L6-PII-9, or L6-PII-13 (19-51% yield) as a white solid.

### 1-[2-(Cyclooct-2-yn-1-yloxy)acetamido]-N-{[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}-3,6,9,12-tetraoxapentadecan-15-amide (L17-PII-1)

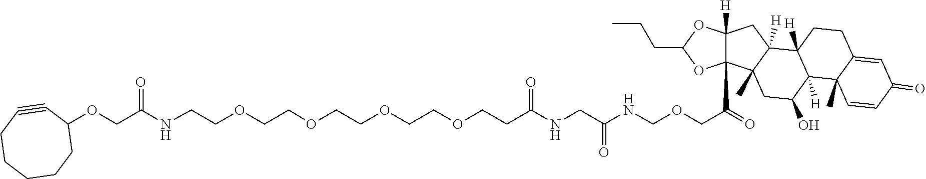 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CCOCCOCCOCCOCCNC(=O)COC2C#CCCCCC2)O1]

Following the General Procedure for pH-sensitive linker-payloads, from L-la and PII-1, the linker-payload L17-PII-1 (30 mg, 22% yield) was obtained as a white solid. ESI m/z: 498.3 (M-M_{Bud}+H)⁺, 950.6 (M+Na)⁺. ¹H NMR (400 MHz, DMSO_{d6}) δ 8.73-8.59 (m, 1H), 8.27-8.15 (m, 1H), 7.60 (t, J=6.0 Hz, 1H), 7.31 (dd, J=10.0, 2.4 Hz, 1H), 6.21-6.11 (m, 1H), 5.92 (s, 1H), 5.20-5.00 (m, 1H), 4.80-4.56 (m, 4H), 4.54-4.43 (m, 1H), 4.34-4.10 (m, 3H), 3.90-3.83 (m, 1H), 3.79-3.66 (m, 3H), 3.61 (t, J=6.4 Hz, 2H), 3.54-3.46 (m, 12H), 3.42 (t, J=6.0 Hz, 2H), 3.28-3.19 (m, 2H), 2.41 (t, J=6.4 Hz, 2H), 2.34-2.15 (m, 3H), 2.14-2.01 (m, 2H), 2.00-1.83 (m, 3H), 1.81-1.72 (m, 4H), 1.64-1.46 (m, 6H), 1.45-1.20 (m, 8H), 1.14-0.90 (m, 2H), 0.89-0.76 (m, 6H) ppm.

### 1-(4-{2-Azatricyclo[10.4.0.0^{4,9}]hexadeca-1(12),4(9),5,7,13,15-hexaen-10-yn-2-yl}-4-oxobutanamido)-N-[2-(2-{2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}pyrrolidin-1-yl)-2-oxoethyl]-3,6,9,12-tetraoxapentadecan-15-amide (L6-PII-9)

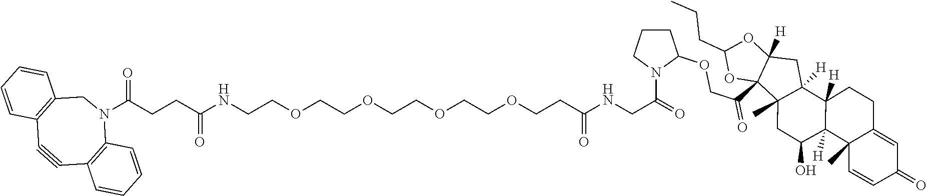 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CNC(=O)CCOCCOCCOCCOCCNC(=O)CCC(=O)N2Cc3ccccc3C#Cc3ccccc32)O1]

Following the General Procedure for pH-sensitive linker-payloads, from L-1b and PII-9, the linker-payload L6-PII-9 (30 mg, 51% yield) was obtained as a white solid. ESI m/z: 661.5 (M-M_{Bud}+H)⁺. ¹H NMR (400 MHz, DMSO_{d6}) δ 8.11-8.02 (m, 1H), 7.77 (t, J=5.2 Hz, 1H), 7.69-7.67 (m, 1H), 7.63-7.61 (m, 1H), 7.52-7.43 (m, 3H), 7.40-7.27 (m, 4H), 6.18-6.15 (m, 1H), 5.92 (s, 1H), 5.44-5.30 (m, 1H), 5.18-5.00 (m, 2H), 4.85-4.48 (m, 3H), 4.33-4.17 (m, 2H), 4.00-3.83 (m, 1H), 3.63-3.53 (m, 3H), 3.48-3.45 (m, 13H), 3.31-3.20 (m, 2H), 3.12-3.03 (m, 2H), 2.62-2.54 (m, 1H), 2.45-2.37 (m, 2H), 2.32-2.20 (m, 2H), 2.10-1.89 (m, 6H), 1.86-1.67 (m, 6H), 1.60-1.42 (m, 4H), 1.38-1.23 (m, 7H), 1.03-0.92 (m, 2H), 0.87-0.81 (m, 6H) ppm.

### 1-(4-{2-Azatricyclo[10.4.0.0^{4,9}]hexadeca-1(12),4(9),5,7,13,15-hexaen-10-yn-2-yl}-4-oxobutanamido)-N-[2-(2-{2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}1 icosa-14,17-dien-8-yl]-2-oxoethoxy}azetidin-1-yl)-2-oxoethyl]-3,6,9,12-tetraoxapentadecan-15-amide (L6-PII-13)

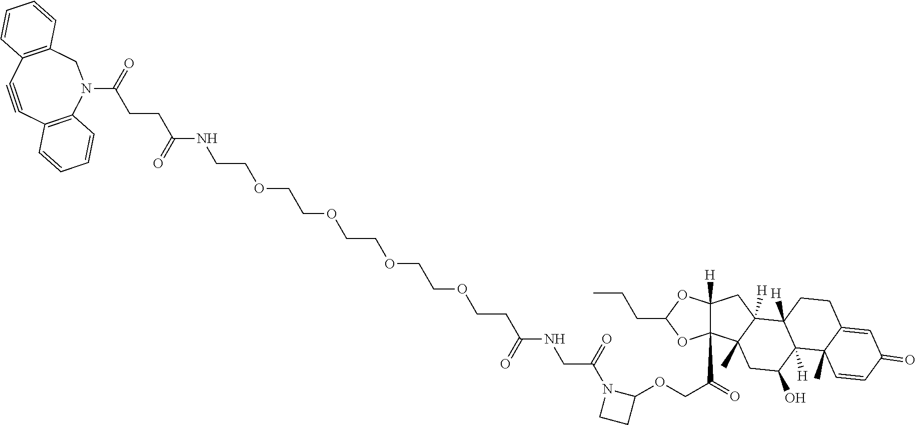 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCN2C(=O)CNC(=O)CCOCCOCCOCCOCCNC(=O)CCC(=O)N2Cc3ccccc3C#Cc3ccccc32)O1]

Following the General Procedure for pH-sensitive linker-payloads, from L-1b and PII-13, the linker-payload L6-PII-13 (20 mg, 19% yield) was obtained as a white solid. ESI m/z: 539.1 (M/2+H)⁺. ¹H NMR (400 MHz, DMSO_{d6}) δ 8.20 (s, 1H), 7.90-7.25 (m, 9H), 6.25-6.20 (m, 1H), 5.95 (s, 1H), 5.20 (s, 1H), 5.10-5.00 (m, 1H), 4.80-4.50 (m, 4H), 4.25-4.20 (m, 3H), 3.70-3.60 (m, 3H), 3.40-3.30 (m, 9H), 3.20-3.15 (m, 3H), 3.20-3.00 (m, 3H), 2.55-2.50 (m, 1H), 2.40-2.20 (m, 4H), 2.00-1.80 (m, 3H), 1.80-1.75 (m, 5H), 1.75-1.50 (m, 4H), 1.45-1.25 (m, 4H), 1.20-1.05 (m, 3H), 0.85-0.75 (m, 8H) ppm.

### 4-{2-Azatricyclo[10.4.0.0^{4,9}]hexadeca-1(12),4(9),5,7,13,15-hexaen-10-yn-2-yl}-N-{[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}-4-oxobutanamide (L7-PII-1)

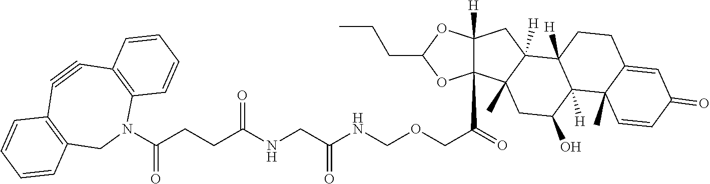 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CCC(=O)N2Cc3ccccc3C#Cc3ccccc32)O1]

Following the General Procedure for pH-sensitive linker-payloads, from L-1e and PII-1, the linker-payload L7-PII-1 (30 mg, 50% yield) was obtained as a white solid. ESI m/z: 804 (M+1)⁺. ¹H NMR (400 MHz, DMSO_{d6}) δ 8.62-8.53 (m, 1H), 8.23-8.11 (m, 1H), 7.69-7.61 (m, 2H), 7.54-7.44 (m, 3H), 7.43-7.26 (m, 4H), 6.20-6.13 (m, 1H), 5.92 (s, 1H), 5.20-5.00 (m, 2H), 4.76-4.40 (m, 5H), 4.32-4.10 (m, 2H), 3.69-3.53 (m, 3H), 2.70-2.60 (m, 1H), 2.35-2.24 (m, 2H), 2.14-1.93 (m, 3H), 1.80-1.70 (m, 3H), 1.62-1.46 (m, 4H), 1.42-1.20 (m, 7H), 1.14-0.90 (m, 2H), 0.87-0.75 (m, 6H) ppm.

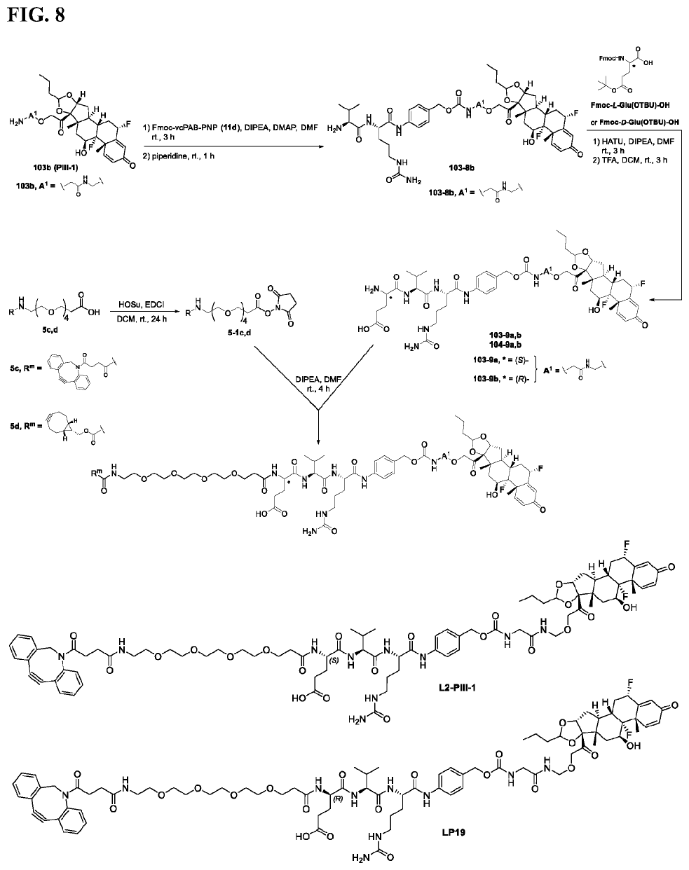

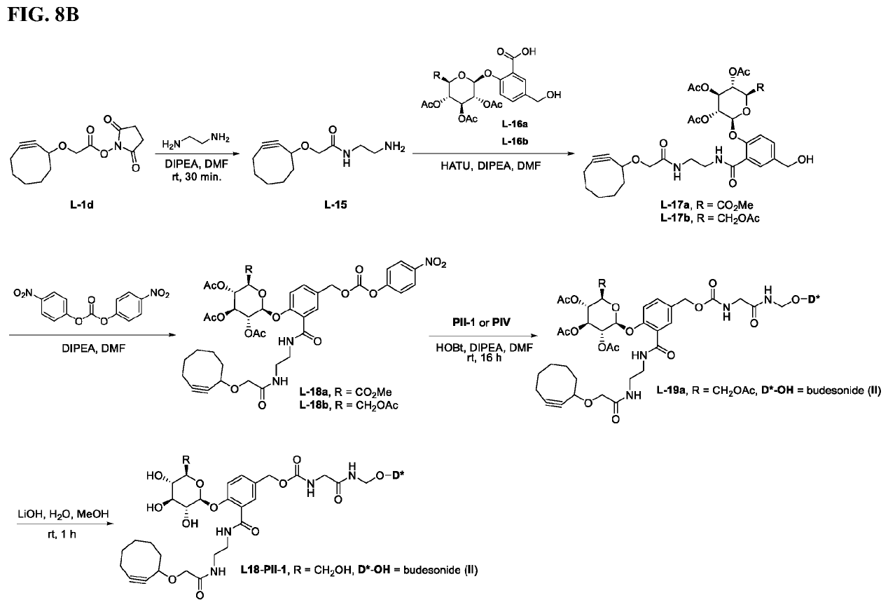

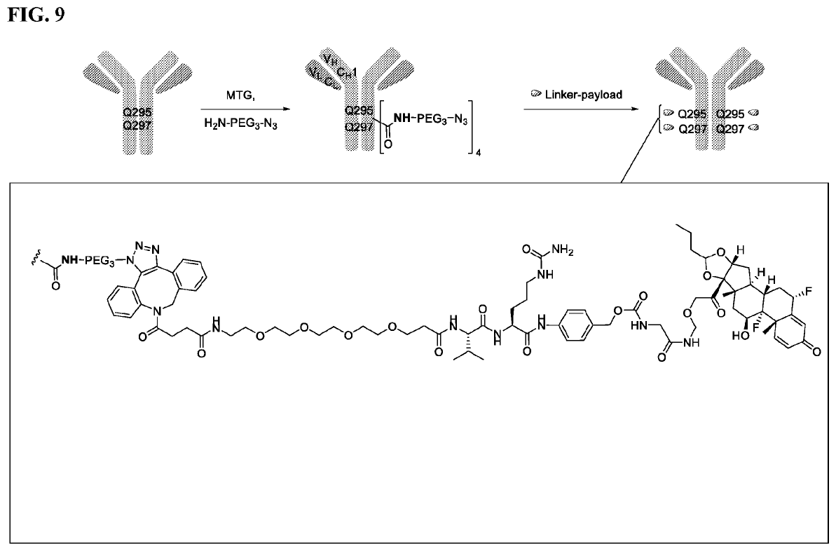

### Synthesis of Chiral Linker-Payloads Including (R)-Glu and (S)-Glu Within L2-PIII-1 (FIG. 8)

### {4-[(2S)-2-[(2S)-2-Amino-3-methylbutanamido]-5-(carbamoylamino)pentanamido]phenyl}methyl N-{[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamate (103-8b)

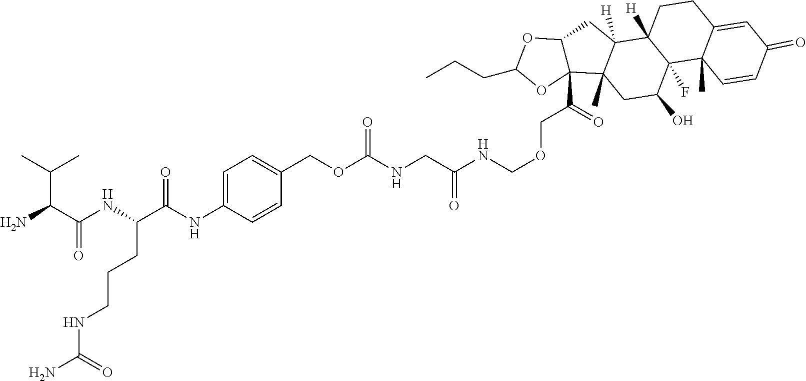 [SMILES: CCCC1O[C@@H]2C[C@H]3[C@@H]4CCC5=CC(=O)C=C[C@]5(C)[C@@]4(F)[C@@H](O)C[C@]3(C)[C@]2(C(=O)COCNC(=O)CNC(=O)OCc2ccc(NC(=O)[C@H](CCCNC(N)=O)NC(=O)[C@@H](N)C(C)C)cc2)O1]

To a solution of crude compound 103b in DMF were added Fmoc-vcPAB-PNP (11d), DMAP, and DIPEA (50 mg, 0.39 mmol) at RT. The mixture was stirred at RT for 3 hours until most of starting materials were consumed, which was monitored by LCMS. To the reaction mixture was then added piperidine. After the reaction was stirred at room temperature for an hour, Fmoc was totally removed according to LCMS. The reaction mixture was directly purified by prep-HPLC (method B) to give compound 103-8b (28 mg, 20% yield) was obtained as a white solid. ESI m/z: 480 (M/2+H)⁺.

### (4S)-4-Amino-4-{[(1S)-1-{[(1S)-4-(carbamoylamino)-1-[(4-{[({[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)oxy]methyl}phenyl)carbamoyl]butyl]carbamoyl}-2-methylpropyl]carbamoyl}butanoic acid (103-9a)

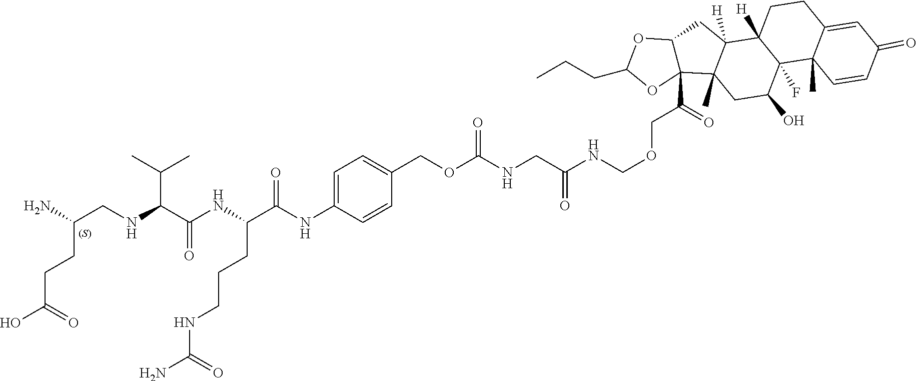

To a solution of compound Boc-L-Glu(OTBU)-OH (0.15 g, 0.50 mmol) in DMF (5 mL) were added HATU (0.19 g, 0.50 mmol) and DIPEA (0.13 g, 1.0 mmol). The reaction mixture was stirred at RT for 10 minutes, before compound 103-8b (0.48 g, 0.50 mmol) was added to the reaction mixture. The reaction mixture was then stirred at RT for 3 hours until 103-8b was totally consumed according to LCMS. The mixture was extracted with EtOAc, and the combined organic solution was washed with water, dried over anhydrous sodium sulfate and concentrated in vacuo. The residue was dissolved into DCM (10 mL). To the solution was added TFA (2 mL), and the reaction mixture was stirred at RT for 3 hours, which was monitored by LCMS. The reaction mixture was concentrated, and the residue was directly purified by prep-HPLC (method B) to give compound 103-9a (0.41 g, 75% yield) as a white solid. ESI m/z: 536.8 (M/2+H)⁺.

### (4R)-4-Amino-4-{[(1S)-1-{[(1S)-4-(carbamoylamino)-1-[(4-{[({[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)oxy]methyl}phenyl) carbamoyl]butyl]carbamoyl}-2-methylpropyl]carbamoyl}butanoic acid (103-9b)

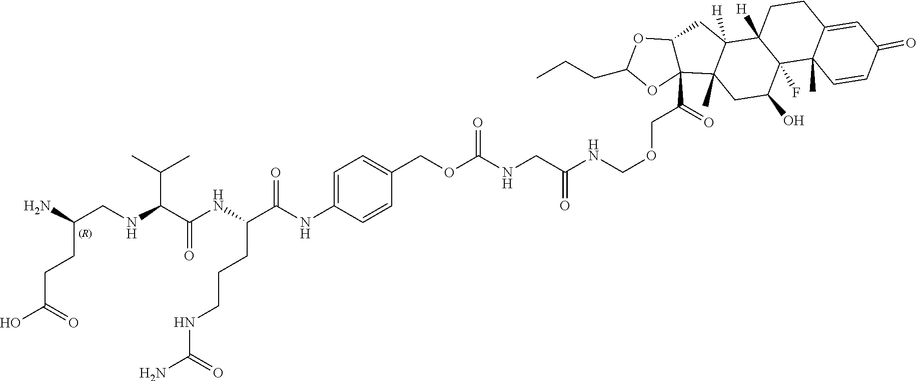

Following the similar procedure as 103-9a except substituting Boc-D-Glu(OTBU)-OH for Boc-L-Glu(OTBU)-OH, compound 103-9b (0.40 g, 74% yield) as a white solid. ESI m/z: 536.8 (M/2+H)⁺.

### 2,5-Dioxopyrrolidin-1-yl 1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-oate (5-1c)

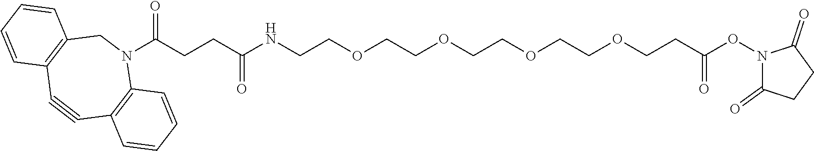 [SMILES: O=C(CCC(=O)N1Cc2ccccc2C#Cc2ccccc21)NCCOCCOCCOCCOCCC(=O)ON1C(=O)CCC1=O]

To a solution of commercially available compound 5c (160 mg, 0.290 mmol) in DCM (20 mL) were added HOSu (73.3 mg, 0.637 mmol) and EDCI (122 mg, 0.637 mmol), and the mixture was stirred at RT for 24 hours, which was monitored by LCMS. The reaction mixture was diluted with DCM (50 mL) and the organic layer was washed with water (50 mL) and brine, dried with anhydrous Na₂SO₄, and concentrated in vacuo to give compound 5-1c (159 mg, 84% yield) as colorless oil, which was used without purification. ESI m/z: 650 (M+H)⁺.

### 2,5-Dioxopyrrolidin-1-yl 1-[({endo-bicyclo[6.1.0]non-4-yn-9-ylmethoxy}carbonyl)amino]-3,6,9,12-tetraoxapentadecan-15-oate (5-1d)

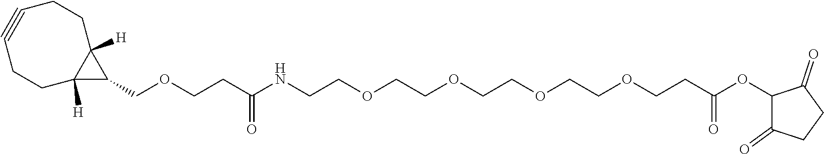 [SMILES: O=C(CCOC[C@@H]1[C@@H]2CCC#CCC[C@@H]21)NCCOCCOCCOCCOCCC(=O)OC1C(=O)CCC1=O]

Following a similar procedure as 5-1c, except substituting 5d for 5c, compound 5-1d (150 mg, 54% yield) was obtained as colorless oil, which was used without further purification. ESI m/z: 539 (M+H)⁺.

### (4S)-4-[i-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-4-{[(1S)-1-{[(1S)-4-(carbamoylamino)-1-[(4-{[({[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)oxy]methyl}phenyl)carbamoyl]butyl]carbamoyl}-2-methylpropyl]carbamoyl}butanoic acid (L2-PIII-1) pop-1141.01

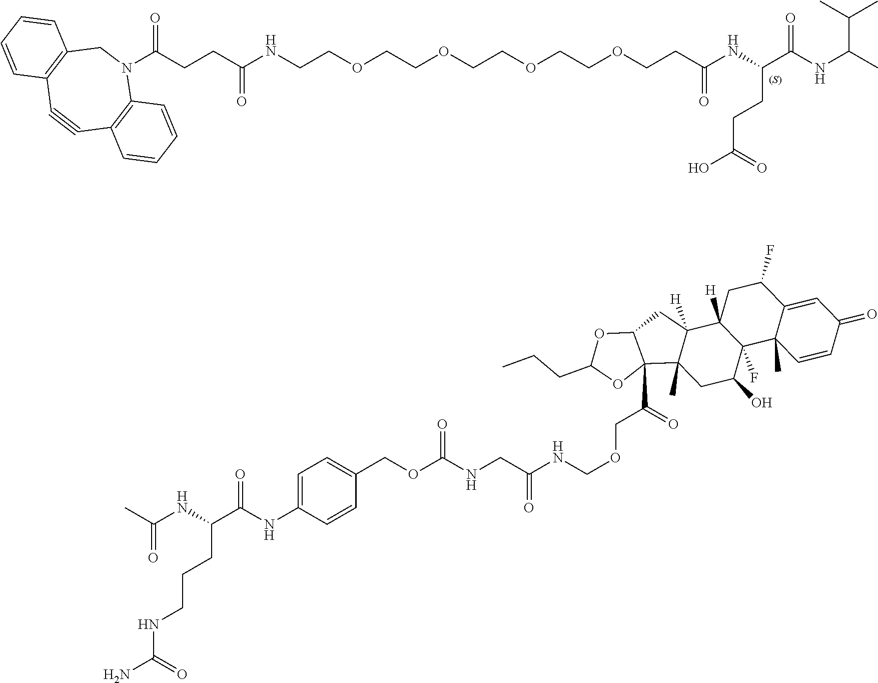

To a solution of compound 103-9a (57 mg, 53 μmol) in DMF (1 mL) were added compound 5-1c (36 mg, 56 μmol) and DIPEA (27 mg, 0.21 mmol). The reaction mixture was stirred at RT for 4 hours, which was monitored by LCMS. The resulting mixture was then directly purified by prep-HPLC (method B) to give compound L2-PIII-1 (12 mg, 15% yield) as a white solid. ESI m/z: 811.4 (M/2+H)⁺.

### (4R)-4-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-4-{[(1S)-1-{[(1S)-4-(carbamoylamino)-1-[(4-{[({[({2-[(1S,2S,4R,8S,9S,11S,12R,13S,19S)-12,19-difluoro-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)oxy]methyl}phenyl)carbamoyl]butyl]carbamoyl}-2-methylpropyl]carbamoyl}butanoic acid (LP19)

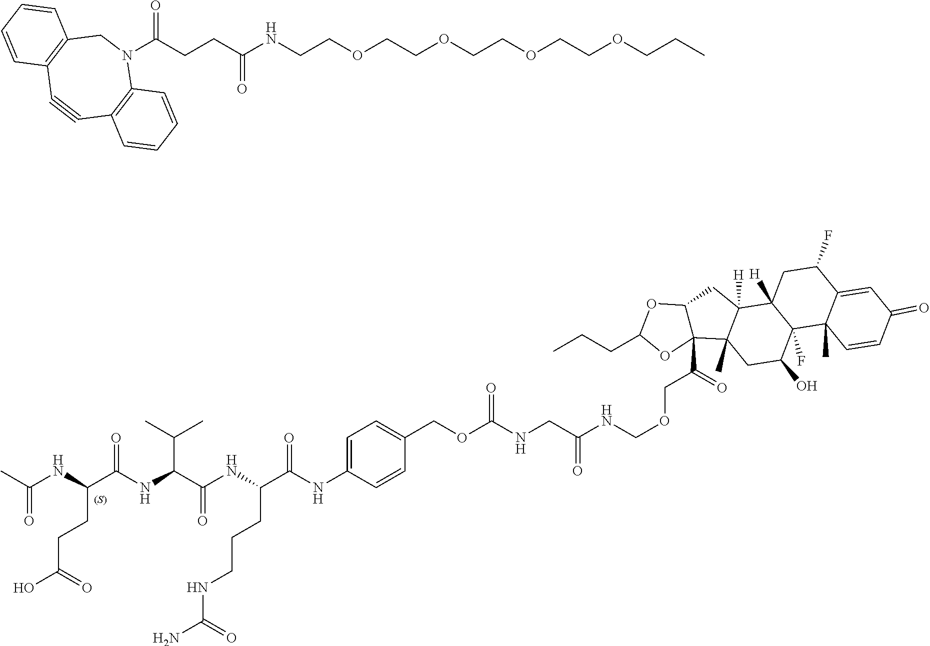

Following a similar procedure as L2-PIII-1, except substituting 103-9b for 103-9a, compound LP19 (14 mg, 17% yield) as a white solid. ESI m/z: 811.4 (M/2+H)⁺.

### Synthesis of Glucose Linker-Payload L18-PII-1 (FIG. 8B)

### N-(2-Aminoethyl)-2-(cyclooct-2-yn-1-yloxy)acetamide (L-15)

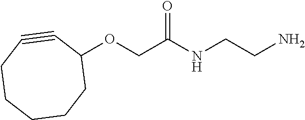 [SMILES: NCCNC(=O)COC1C#CCCCCC1]

To a solution of ethylenediamine (0.71 g, 12 mmol) in DMF (2.0 mL) were added DIPEA (0.30 g, 2.4 mmol) and a solution of compound L-1d (0.33 g, 1.2 mmol) in DMF (3.0 mL) slowly, and the mixture was stirred at room temperature for 30 min, which was monitored by LCMS. The resulting mixture was purified by reversed phase flash chromatography (0-100% acetonitrile in aq. ammonium bicarbonate (0.8 mM)) to give compound L-15 (0.18 g, 68% yield) as colorless oil. ESI m/z: 225.2 (M+H)⁺. ¹H NMR (400 MHz, DMSO_{d6}) δ 7.74-7.63 (m, 1H), 4.28 (t, J=5.8 Hz, 1H), 3.88-3.73 (m, 2H), 3.11-3.00 (m, 4H), 2.58 (t, J=6.4 Hz, 2H), 2.27-2.06 (m, 3H), 1.94-1.71 (m, 4H), 1.66-1.54 (m, 2H), 1.45-1.33 (m, 1H) ppm.

### Methyl (2S,3S,4S,5R,6S)-3,4,5-tris(acetyloxy)-6-[2-({2-[2-(cyclooct-2-yn-1-yloxy)acetamido]ethyl}carbamoyl)-4-(hydroxymethyl)phenoxy]oxane-2-carboxylate (L-17a)

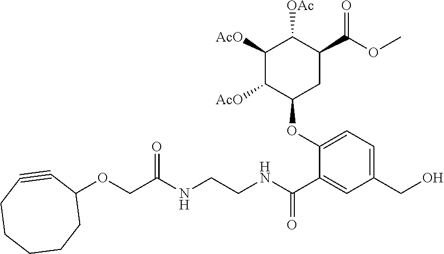 [SMILES: COC(=O)[C@H]1C[C@@H](Oc2ccc(CO)cc2C(=O)NCCNC(=O)COC2C#CCCCCC2)[C@H](OC(C)=O)[C@@H](OC(C)=O)[C@@H]1OC(C)=O]

To a mixture of compound L-16a (synthesized according to WO 2018/182341 A1) (0.11 g, 0.23 mmol) and HATU (96 mg, 0.25 mmol) in dry DMF (4 mL) were added compound L-15 (51 mg, 0.23 mmol) and DIPEA (89 mg, 0.69 mmol), and the reaction mixture was stirred at room temperature for 2 hours until L-16a was totally consumed, as monitored by LCMS. The resulting mixture was directly purified by reversed phase flash chromatography (0-100% acetonitrile in aq. TFA (0.01%)) to give compound L-17a (0.14 g, 90% yield) as a white solid. ESI m/z: 691.4 (M+H)⁺. ¹H NMR (400 MHz, CDCl₃) δ 8.06-8.04 (m, 1H), 7.64-7.59 (m, 1H), 7.50-7.47 (m, 1H), 7.22-7.18 (m, 1H), 7.01-6.98 (m, 1H), 5.44-5.28 (m, 5H), 4.68 (s, 2H), 4.30-4.21 (m, 2H), 4.10-4.06 (m, 1H), 3.93-3.88 (m, 1H), 3.75 (s, 3H), 3.67-3.48 (m, 2H), 2.21-2.07 (m, 15H), 1.93-1.79 (m, 3H), 1.70-1.38 (m, 3H) ppm.

### [(2R,3R,4S,5R,6S)-3,4,5-Tris(acetyloxy)-6-[2-({2-[2-(cyclooct-2-yn-1-yloxy)acetamido]ethyl}carbamoyl)-4-(hydroxymethyl)phenoxy]oxan-2-yl]methyl acetate

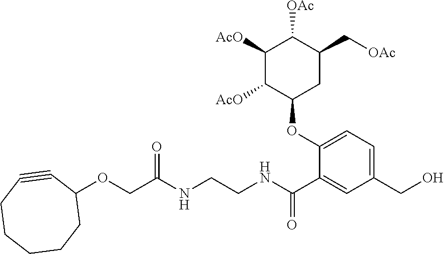 [SMILES: CC(=O)OC[C@H]1C[C@@H](Oc2ccc(CO)cc2C(=O)NCCNC(=O)COC2C#CCCCCC2)[C@H](OC(C)=O)[C@@H](OC(C)=O)[C@@H]1OC(C)=O]

Following a similar procedure as L-17a, except substituting L-16b for L-16a, compound L-17b (0.10 g, 80% yield) was obtained as a white solid. ESI m/z: 705.3 (M+H)⁺.

### Methyl (2S,3S,4S,5R,6S)-3,4,5-tris(acetyloxy)-6-[2-({2-[2-(cyclooct-2-yn-1-yloxy)acetamido]ethyl}carbamoyl)-4-{[(4-nitrophenoxycarbonyl)oxy]methyl}phenoxy]oxane-2-carboxylate (L-18a)

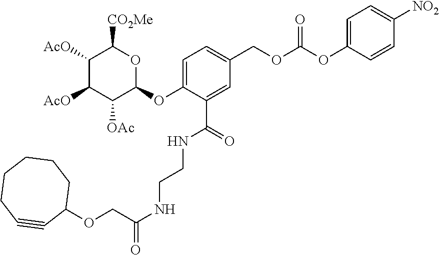 [SMILES: COC(=O)[C@H]1O[C@@H](Oc2ccc(COC(=O)Oc3ccc([N+](=O)[O-])cc3)cc2C(=O)NCCNC(=O)COC2C#CCCCCC2)[C@H](OC(C)=O)[C@@H](OC(C)=O)[C@@H]1OC(C)=O]

To a solution of compound L-17a (0.14 g, 0.20 mmol) in DMF (2.0 mL) were added bis(4-nitrophenyl) carbonate (55 mg, 0.18 mmol) and DIPEA (26 mg, 0.20 mmol) at 0° C. under nitrogen. The reaction mixture was stirred at 0° C. for 30 min, and then at room temperature for 3 hours. The reaction mixture was diluted with water (10 mL) and extracted with ethyl acetate (20 mL×3). The combined organic solution was washed with brine (10 mL), dried over anhydrous sodium sulfate, and concentrated in vacuo. The residue was purified by flash chromatography (40-60% ethyl acetate in petroleum ether) to give compound L-18a (85 mg, 49% yield) as colorless oil. ESI m/z: 856.0 (M+H)⁺.

### [(2R,3R,4S,5R,6S)-3,4,5-Tris(acetyloxy)-6-[2-({2-[2-(cyclooct-2-yn-1-yloxy)acetamido]ethyl}carbamoyl)-4-{[(4-nitrophenoxycarbonyl)oxy]methyl}phenoxy]oxan-2-yl]methyl acetate (L-18b)

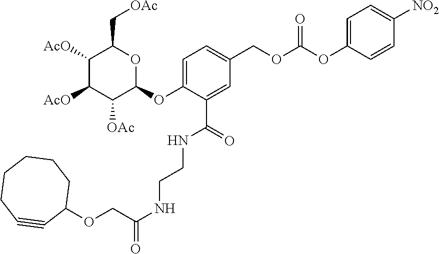 [SMILES: CC(=O)OC[C@H]1O[C@@H](Oc2ccc(COC(=O)Oc3ccc([N+](=O)[O-])cc3)cc2C(=O)NCCNC(=O)COC2C#CCCCCC2)[C@H](OC(C)=O)[C@@H](OC(C)=O)[C@@H]1OC(C)=O]

Following a similar procedure as L-18a, except substituting L-17b for L-17a, compound L-18b (62 mg, 50% yield) was obtained as a white solid. ESI m/z: 870.3 (M+H)⁺.

### [(2R,3R,4S,5R,6S)-3,4,5-Tris(acetyloxy)-6-[2-({2-[2-(cyclooct-2-yn-1-yloxy)acetamido]ethyl}carbamoyl)-4-{[({[({2-[(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamoyl)oxy]methyl}phenoxy]oxan-2-yl]methyl acetate (L-19a)

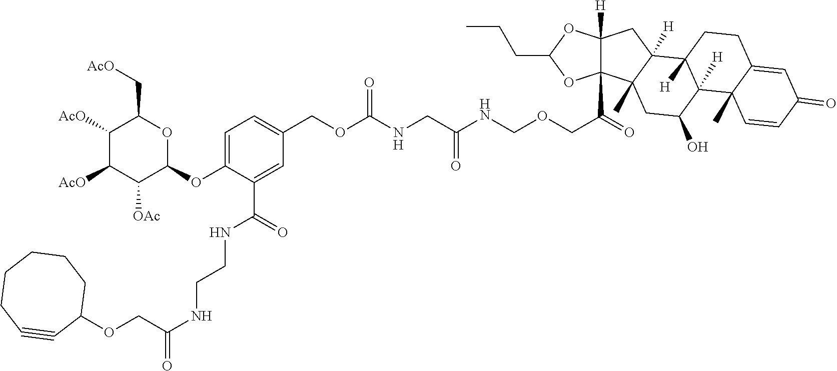

To a solution of compound L-18b (59 mg, 68 μmol) in DMF (4.0 mL) were added payload PII-1 (35 mg, 68 μmol), HOBt (9.1 mg, 68 μmol), and DIPEA (26 mg, 0.20 mmol). The reaction mixture was stirred at room temperature for 16 hours. The reaction mixture was directly purified by reversed phase flash chromatography (0-100% acetonitrile in aq. ammonium bicarbonate (10 mM)) to give compound L-19a (25 mg, 30% yield) as a white solid. ESI m/z: 817.3 (M-M_{Bud}+H)⁺.

### [3-({2-[2-(Cyclooct-2-yn-1-yloxy)acetamido]ethyl}carbamoyl)-4-{[(2S,3R,4S,5S,6R)-3,4,5-trihydroxy-6-(hydroxymethyl)oxan-2-yl]oxy}phenyl]methyl N-{[({2-1(1S,2S,4R,8S,9S,11S,12S,13R)-11-hydroxy-9,13-dimethyl-16-oxo-6-propyl-5,7-dioxapentacyclo[10.8.0.0^{2,9}.0^{4,8}.0^{13,18}]icosa-14,17-dien-8-yl]-2-oxoethoxy}methyl)carbamoyl]methyl}carbamate (L18-PII-1)

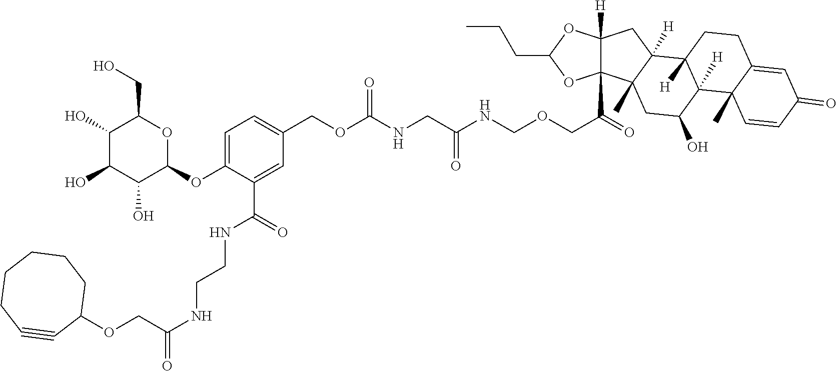

To a mixture of compound L-19a (20 mg, 16 μmol) in methanol (2 mL) was added aq. lithium hydroxide (72 mM, 2 mL), and the mixture was stirred at 15° C. for an hour, which was monitored by LCMS. The reaction mixture was acidified by aq. hydrochloride (1 N) to pH 3 to 4, and was then purified by prep-HPLC (5-95% acetonitrile in aq. formic acid (0.1%)) to give linker-payload L18-PII-1 (10 mg, 45% yield) as a white solid. ESI m/z: 1101.6 (M+Na)⁺. ¹H NMR (400 MHz, DMSO_{d6}) δ 8.85-8.78 (m, 1H), 8.53-8.40 (m, 3H), 7.85 (s, 1H), 7.81-7.74 (m, 1H), 7.64-7.53 (m, 1H), 7.47-7.43 (m, 1H), 7.36-7.30 (m, 2H), 6.16 (d, J=10.0 Hz, 1H), 5.92 (s, 1H), 5.19-5.00 (m, 3H), 4.90 (d, J=6.8 Hz, 1H), 4.81-4.71 (m, 2H), 4.64-4.46 (m, 4H), 4.33-4.14 (m, 3H), 3.90-3.73 (m, 3H), 3.61 (d, J=5.6 Hz, 2H), 3.55-3.50 (m, 2H), 3.29-3.16 (m, 9H), 2.38-1.82 (m, 10H), 1.76-1.48 (m, 10H), 1.38-1.24 (m, 7H), 1.00-0.81 (m, 6H) ppm.

### Example 1

This Example demonstrates specific procedures for site-specific conjugation of an alkyne-containing linker-payload to an antibody.

In this example, the site-specific antibody-drug conjugates can be produced in two steps. In the first step, an azido linking moiety, such as azide-PEG₃-amine is enzymatically attached to the antibody having a glutamine (e.g., Q-tag) via microbial transglutaminase (MTG) (e.g., MTG EC 2.3.2.13, Zedira, Darmstadt, Germany; see WO 2017/147542, which is incorporated herein in its entirety). The site-specific antibody-drug conjugates having a DAR of two can be synthesized by attaching the linking moieties at the Q295 residues of an antibody having an N297D mutation. Site-specific antibody-drug conjugates having a DAR of four can be synthesized by attaching the linking moieties to antibodies having N297Q mutations. The Q295 and Q297 glutamines react with the linking moieties via MTG to provide a DAR of four. The second step employs the attachment of a linker-payload to the azido-functionalized antibody via a [2+3] cycloaddition, for example, a 1,3-dipolar cycloaddition between the azides and the cyclooctynes (aka copper-free click chemistry). See, Baskin, J. M.; Prescher, J. A.; Laughlin, S. T.; Agard, N. J.; Chang, P. V.; Miller, I. A.; Lo, A.; Codelli, J. A.; Bertozzi, C. R. PNAS 2007, 104 (43), 16793-7, which is incorporated herein by reference in its entirety for all purposes. Where the reactive group (RG) is a DIBAC moiety, the conjugation is carried out with an azido-functionalized antibody via a [2+3] cycloaddition. This process provides the site-specific and stoichiometric conjugates.

### Synthesis of a 2DAR Antibody-Drug Conjugate

### Step 1: Preparation of an Azido-Functionalized Antibody

Aglycosylated human antibody IgG (IgG1, IgG4, etc.) or a human IgG1 isotype with N297Q mutation (EU numbering), in PBS (pH 6.5-8.0) is mixed with ≥200 molar equivalents of azido-dPEG₃-amine (MW=218.26 g/mol). The resulting solution is mixed with MTG (EC 2.3.2.13 from Zedira, Darmstadt, Germany, or Modernist Pantry [L #210115A]—ACTIVA TI contains Maltodextrin from Ajinomoto, Japan) (25 U/mL; 5U MTG per mg of antibody) resulting in a final concentration of the antibody at 0.5-5 mg/mL, and the solution is then incubated at 37° C. for 4-24 h while gently shaking. The reaction is monitored by ESI-MS. Upon reaction completion, the excess amine and MTG are removed by SEC or protein A column chromatography, to generate the azido-functionalized antibody. This product is characterized by SDS-PAGE and ESI-MS. The azido-dPEG₃-amine added to two sites of the antibody resulting in a 204 Da increase for the 2DAR antibody-PEG₃-azide conjugate.

### Step 2: Reaction of Azido-Functionalized Antibodies with Linker-Payloads Via Click Chemistry

The site-specific antibody drug conjugates with a human IgG (IgG1, IgG4, etc.) are prepared by a [2+3]click reaction between azido-functionalized antibodies and an alkyne-containing linker-payload. The detailed conjugation procedure is as follows. mAb-PEG₃-N₃ (1-3 mg/mL) is incubated in an aqueous medium (e.g., PBS, PBS containing 5% glycerol, HBS) with ≥6 molar equivalents of an LP dissolved in a suitable organic solvent, such as DMSO, DMF, or DMA (i.e., the reaction mixture contains 5-20% organic solvent, v/v) at 24° C. to 37° C. for over 6 h. The progress of the reaction is monitored by ESI-MS and the absence of mAb-PEG₃-N₃ indicates the completion of the conjugation. The excess amount of the LP and organic solvent are removed by SEC via elution with PBS, or via protein A column chromatography via elution with acidic buffer followed by neutralization with Tris (pH 8.0). The final product is concentrated by ultra-centrifugation and characterized by UV, SEC, SDS-PAGE, and ESI-MS.

### Synthesis of a 4DAR Antibody-Drug Conjugate

### Step 1: Preparation of an Azido-Functionalized Antibody

Aglycosylated antibody with a human IgG1 isotype in BupH™ (pH 7.6-7.8) is mixed with ≥200 molar equivalents of azido-dPEG₃-amine (MW 218.26 g/mol). The resulting solution is then mixed with transglutaminase (25 U/mL; 5U MTG per mg of antibody) resulting in a final concentration of the antibody at 0.5-3 mg/mL, and the solution is then incubated at 37° C. for 4-24 hours while gently shaking. The reaction is monitored by SDS-PAGE or ESI-MS. Upon reaction completion, the excess amine and MTG can be removed by Size Exclusion Chromatography (SEC) to generate the azido-functionalized antibody. This product can be analyzed via SDS-PAGE and ESI-MS. The azido-dPEG₃-amine adds to two sites, Q295 and Q297, of the antibody resulting in an 804 Da increase for the 4DAR aglycosylated antibody-PEG₃-azide conjugate.

### Step 2: Reaction of Azido-Functionalized Antibodies with Linker-Payloads Via Click Chemistry

mAb-PEG₃-N₃ (1-3 mg/mL) is incubated in an aqueous medium (e.g., PBS, PBS containing 5% glycerol, HBS) with ≥6 molar equivalents of an linker-payload dissolved in a suitable organic solvent, such as DMSO, DMF, or DMA (i.e., the reaction mixture contains 5-20% organic solvent, v/v) at 24° C. to 37° C. for over 6 h. The progress of the reaction can be monitored by ESI-MS and the absence of mAb-PEG₃-N₃ indicates completion of the conjugation. The excess amount of the linker-payload and organic solvent can be removed by SEC via elution with PBS, or via protein A column chromatography via elution with acidic buffer followed by neutralization with Tris (pH8.0). The purified conjugates can be analyzed by SEC, SDS-PAGE, and ESI-MS.

### Example 2

This example demonstrates a method for making non-site-specific conjugations of a drug to an antibody using a thiol-maleimide Michael addition reaction. Conjugation through antibody cysteines is performed in two steps using similar methods as described in Mol Pharm. 2015 Jun. 1; 12(6):1863-71, which is incorporated by reference herein in its entirety.

A monoclonal antibody (mAb, 10 mg/mL in 50 mM HEPES, 150 mM NaCl) at pH 7.5 is reduced with 1 mM dithiothreitol (0.006 mg per mg of antibody) or TCEP (2.5 molar equivalents to antibody) at 37° C. for 30 minutes. The concentration of the antibody can be calculated based on the absorbance at 280 nm on a Nanodrop (ThermoFisher Scientific) and using an extinction coefficient of the antibody. After gel filtration (G-25, pH 4.5 sodium acetate), the linker-payload compound in DMSO (10 mg/mL) is added to the reduced antibody, and the mixture is adjusted to pH 7.0 with 1 M HEPES (pH 7.4). The reaction is allowed to progress for 3-14 hours. The resulting conjugate can be purified by SEC. The DAR (UV) values are determined using the measured absorbances of the ncADC and the extinction coefficients of the antibody and linker-payload compound.

### Example 3

### Characterization of ADCs

SDS-PAGE can be used to analyze the integrity and purity of the ADCs.

In one method, SDS-PAGE conditions include non-reduced and reduced samples (2-4 μg) along with BenchMark Pre-Stained Protein Ladder (Invitrogen, cat #10748-010; L #1671922) are loaded per lane in (1.0 mm×10 well) Novex 4-20% Tris-Glycine Gel and is run at 180 V, 300 mA, for 80 minutes. A non-reduced sample is prepared using Novex Tris-Glycine SDS buffer (2×) (Invitrogen, Cat #LC2676) and the reduced sample is prepared with SDS sample buffer (2×) containing 10% 2-mecaptoethanol.

Molecular weights of the antibodies and ncADCs on SDS-PAGE are determined under non-reducing and reducing conditions. The mass shifts may not be obvious under non-reducing conditions due to relatively small percentages of mass changes. However, the masses of the heavy chains are increased from the naked antibodies to the azido-functionalized antibodies, and further for the ncADC conjugate. Cross-linked material may or may not be detected.

ADCs are analyzed for purity by Size Exclusion Chromatography (SEC).

To determine the purity of antibody-drug conjugates, size exclusion chromatography is performed. Analytical SEC experiments are run using a Waters 600 instrument, on a Superdex 200 (1.0×30 cm) HR column, at flow rate of 0.80 mL/min using PBS pH 7.4, and monitored at λ280 nm using a Waters 2998 PDA. An analytic sample is composed of 200 μL PBS (pH 7.4) with 30-100 μL of test sample. Preparative SEC purifications are performed using an AKTA instrument from GE Healthcare, on Superdex 200 PG (2.6×60 cm) column, at a flow rate 2 mL/min eluting with PBSg at pH 7.4, and monitored at λ280 nm.

Antibody and ADC are analyzed by intact mass analysis via LC-ESI-MS, as described further below.

Measurement of intact mass for the ADC samples by LC-ESI-MS can be performed to determine drug-payload distribution profiles and to calculate the average DAR. Each testing sample (20-50 ng, 5 μL) can be loaded onto an Acquity UPLC Protein BEH C4 column (1-OK psi, 300 Å, 1.7 μm, 75 μm×100 mm; Cat No. 186003810). After 3 min. desalting, the protein is eluted and mass spectra can be acquired by a Waters Synapt G2-Si mass spectrometer.

### Example 4

### General Procedure for Quenching Linker-Payloads

To conduct model studies on the linker-payloads described herein, e.g., pH stability tests, capB assays, chemical stability tests, and a plasma stability assays, “quenched linker-payloads” were synthesized as surrogates. To a solution of linker-payloads (2 mg) in acetonitrile and water (v/v=1, 0.4 mL) were added DIPEA (5 equiv.) and azide (CD-N₃ or taurine-PEG4-azide; 3 equiv.) successively. The reaction mixture was stirred at RT for 20 hours, and monitored by LCMS. The reaction mixture was directly purified by prep-HPLC to give quenched linker-payloads. For example, L1-PII-1, L1-PII-6, L1-PII-7, L1-PII-8, and L1-PII-9 were treated with Taurine-PEG₄-N₃ to give the corresponding quenched linker-payloads (Q1L1-PII-#); and L1-PII-1, L6-PII-1, and L7-PII-1 were treated with CD-N₃ to give the corresponding quenched linker-payloads (Q2L1-PII-1, Q2L6-PII-1, and Q2 L7-PII-1). All quenched linker-payloads are listed in Table 3d and/or Table 6 below.

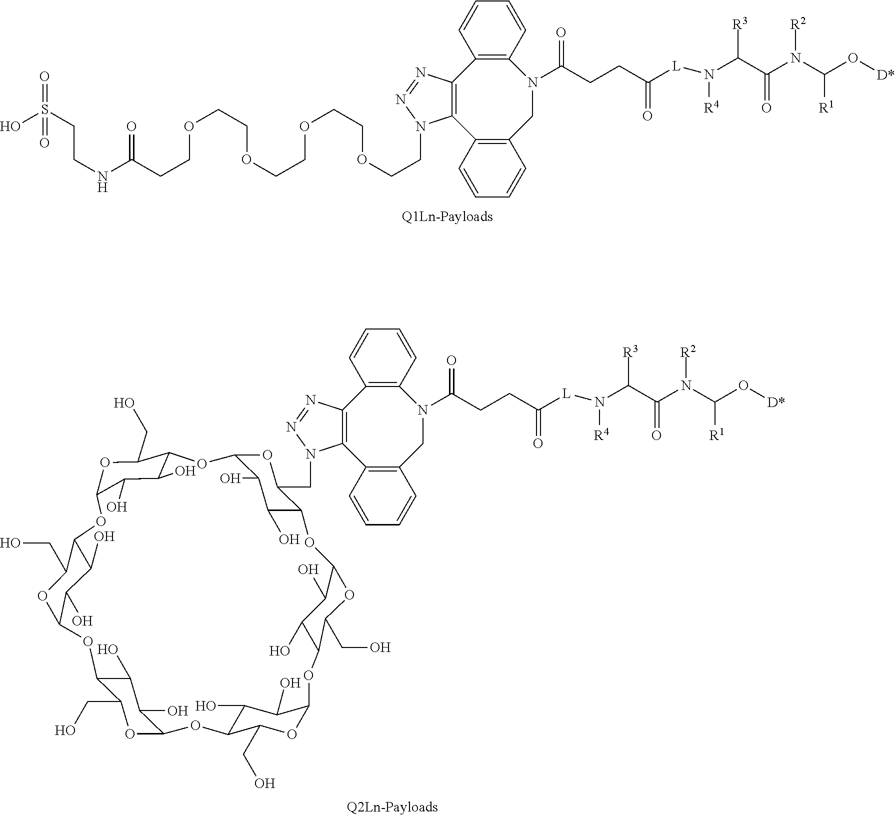

TABLE 6

| QLP# | Structures |
|---|---|
| Q1L1- PI | 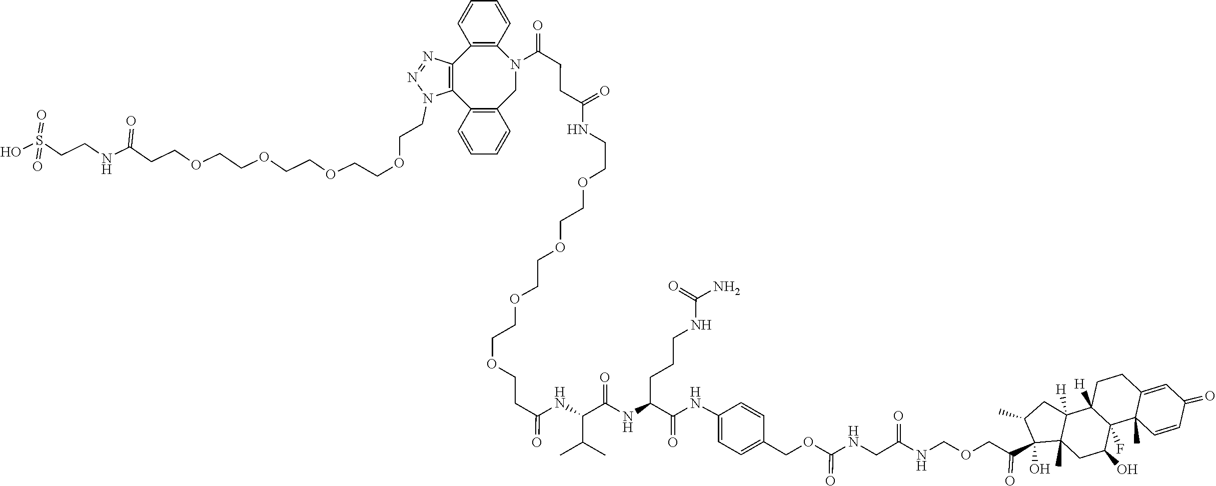 [SMILES: CC(C)[C@H](NC(=O)CCOCCOCCOCCOCCNC(=O)CCC(=O)N1Cc2ccccc2-c2c(nnn2CCOCCOCCOCCOCCC(=O)NCCS(=O)(=O)O)-c2ccccc21)C(=O)N[C@@H](CCCNC(N)=O)C(=O)Nc1ccc(COC(=O)NCC(=O)NCOCC(=O)[C@@]2(O)[C@H](C)C[C@H]3[C@@H]4CCC5=CC(=O)C=C[C@]5(C)[C@@]4(F)[C@@H](O)C[C@@]32C)cc1] |
| | 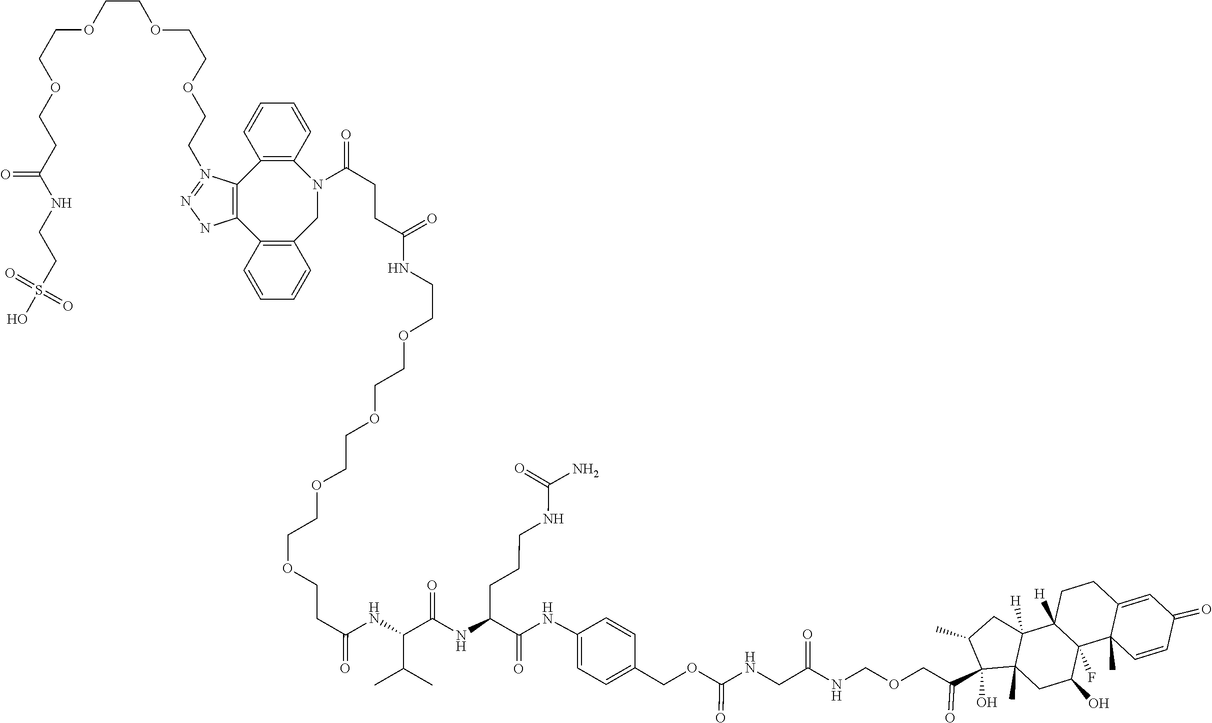 |
| Q1L1- PII-1 | 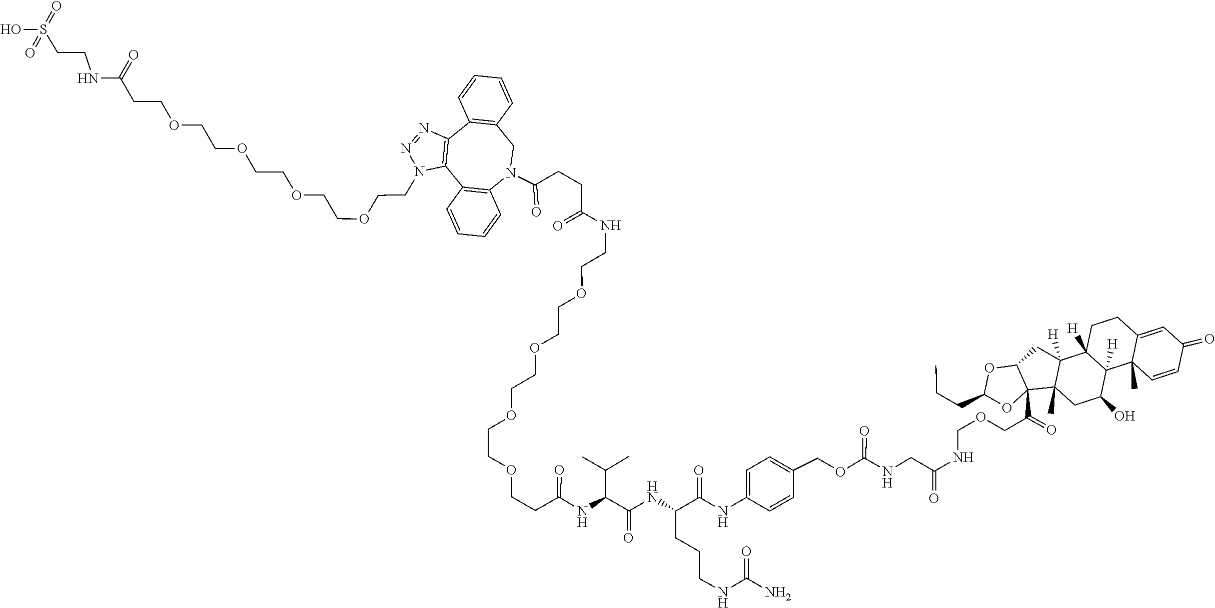 |
| | 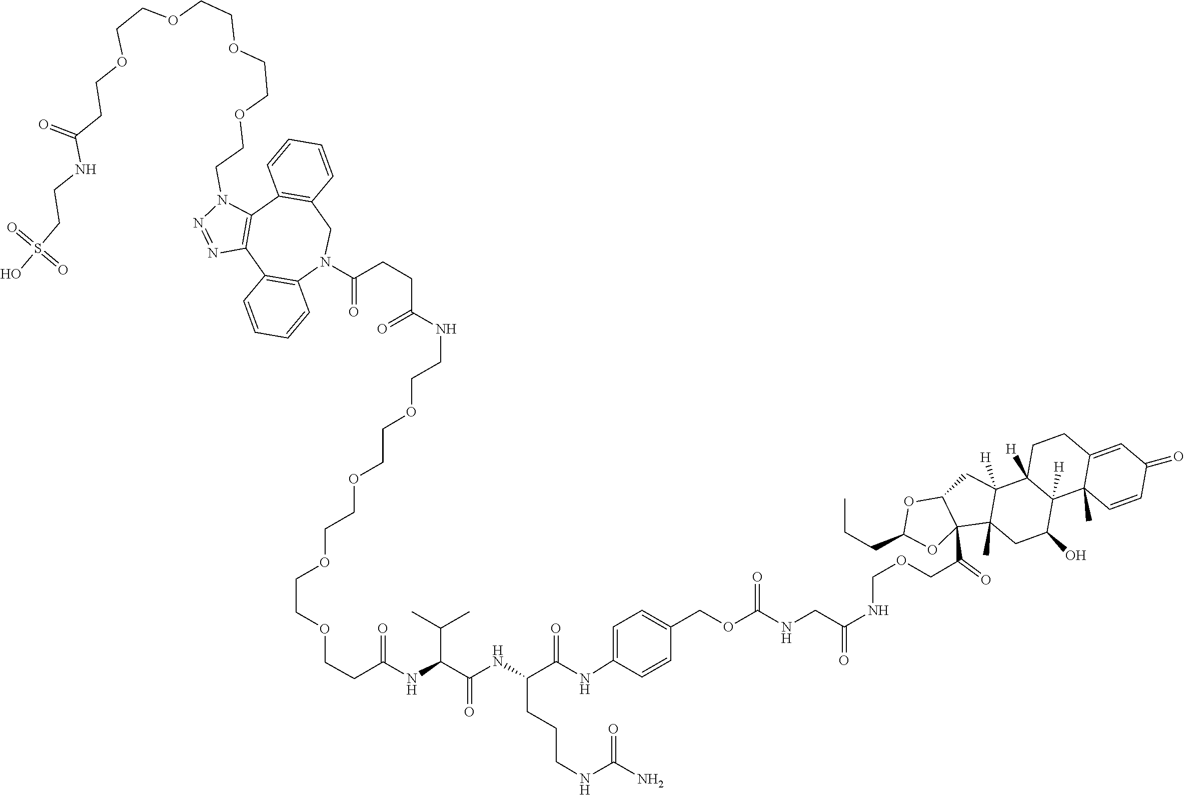 |
| Q1L1- PII-2 | 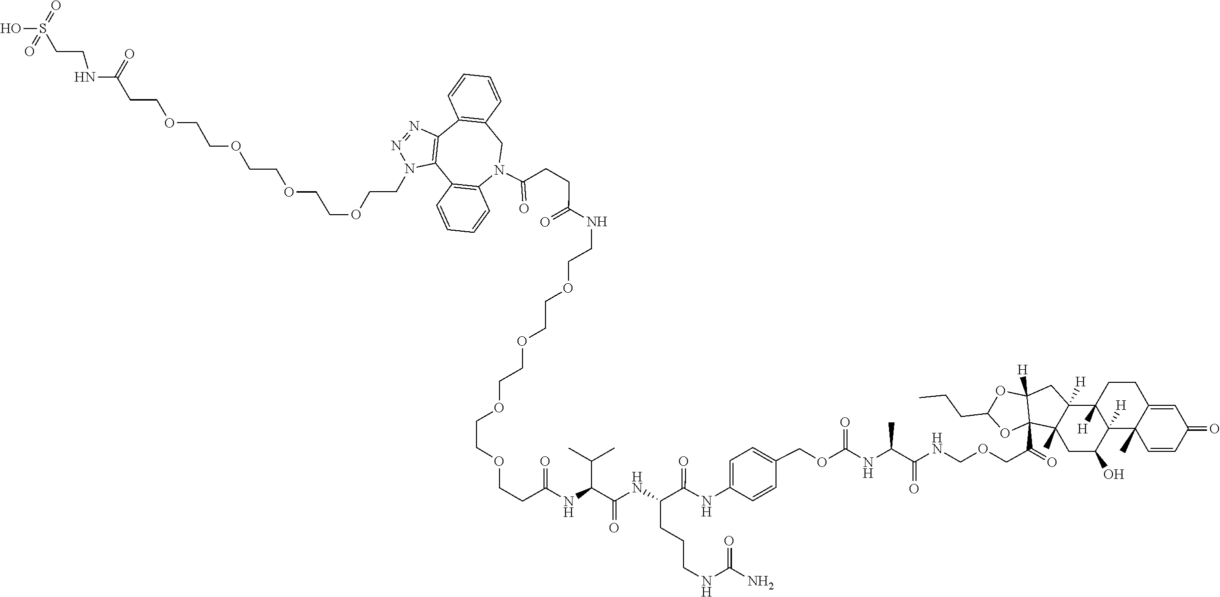 |
| | 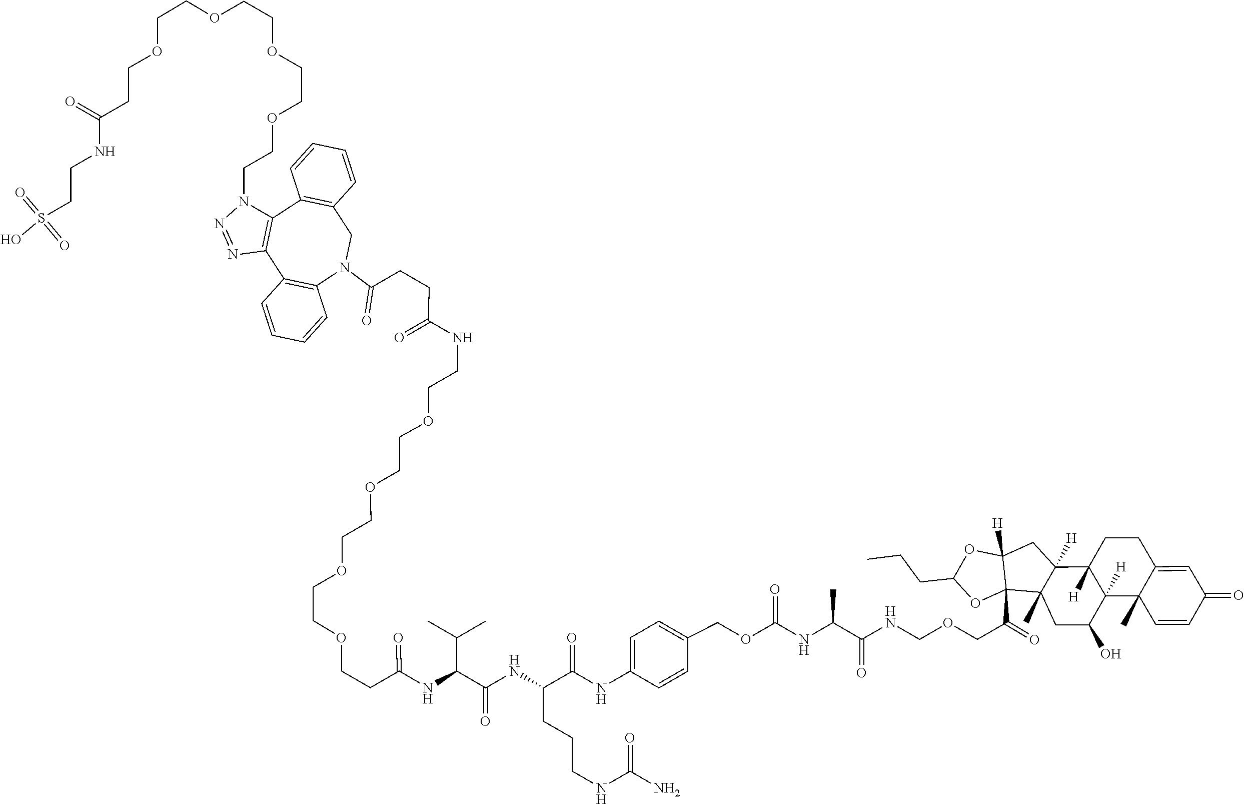 |
| Q1L1- PII-3 | 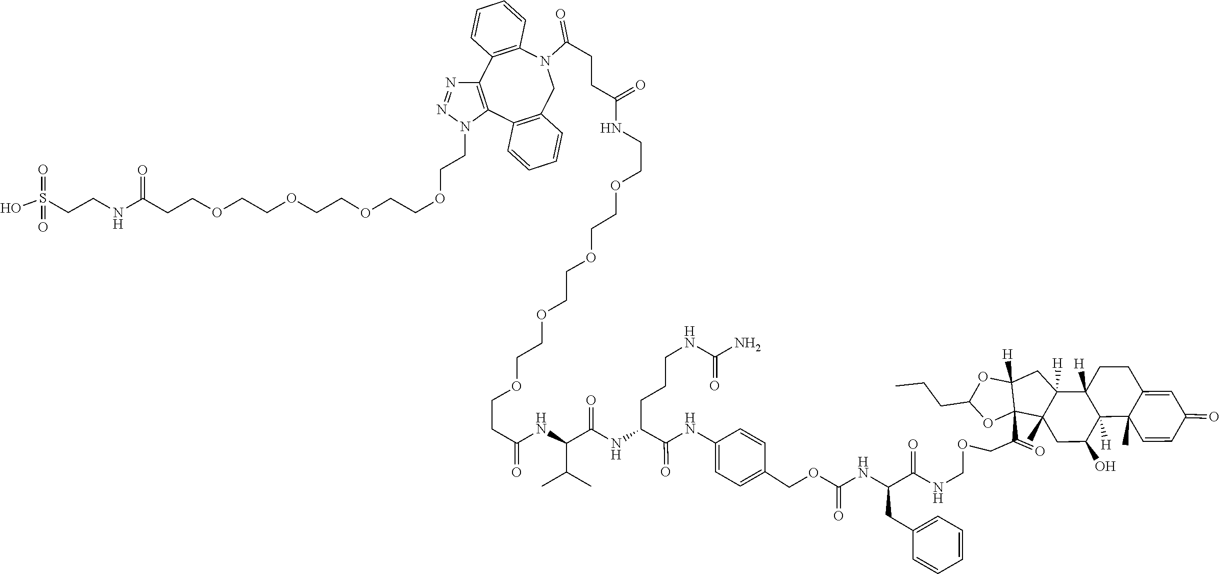 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)[C@@H](Cc2ccccc2)NC(=O)OCc2ccc(NC(=O)[C@@H](CCCNC(N)=O)NC(=O)[C@H](NC(=O)CCOCCOCCOCCOCCNC(=O)CCC(=O)N3Cc4ccccc4-c4c(nnn4CCOCCOCCOCCOCCC(=O)NCCS(=O)(=O)O)-c4ccccc43)C(C)C)cc2)O1] |
| | 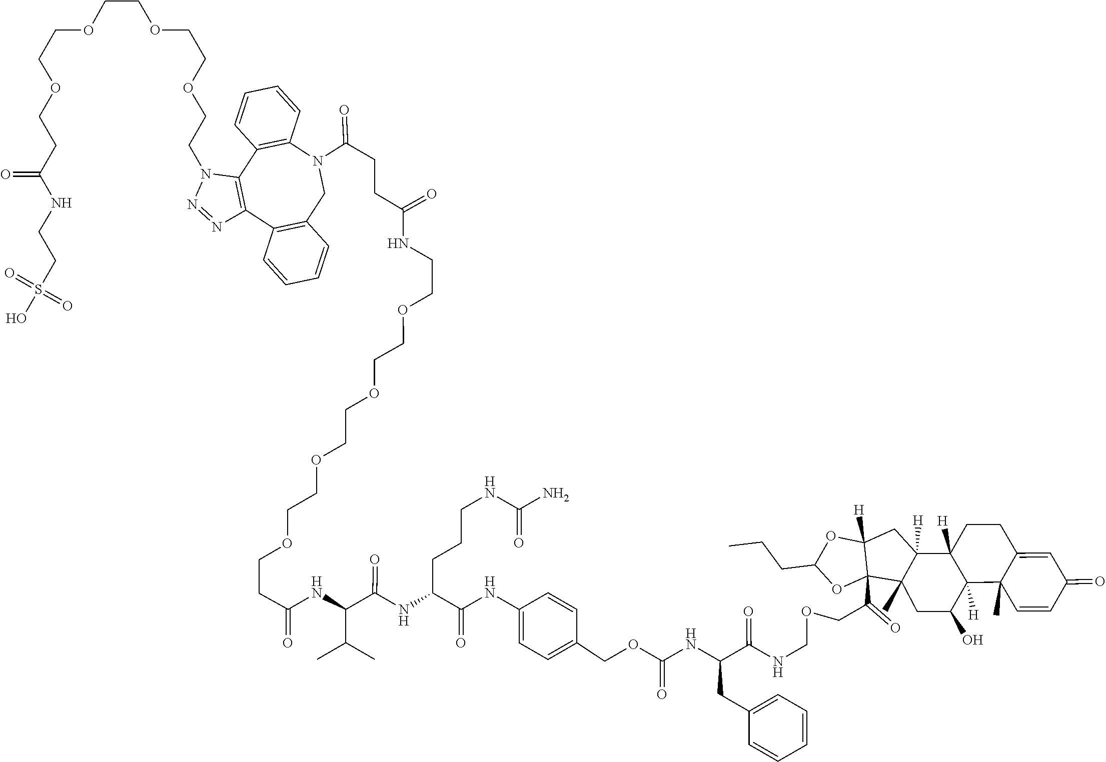 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)[C@@H](Cc2ccccc2)NC(=O)OCc2ccc(NC(=O)[C@@H](CCCNC(N)=O)NC(=O)[C@H](NC(=O)CCOCCOCCOCCOCCNC(=O)CCC(=O)N3Cc4ccccc4-c4nnn(CCOCCOCCOCCOCCC(=O)NCCS(=O)(=O)O)c4-c4ccccc43)C(C)C)cc2)O1] |
| Q1L1- PII-4 | 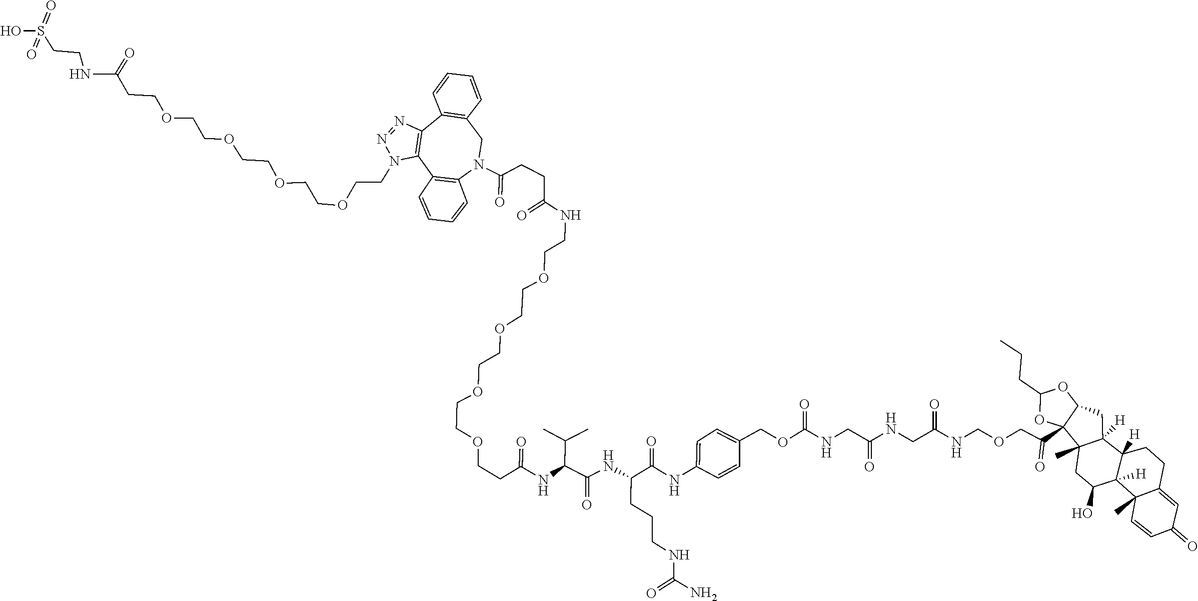 |
| | 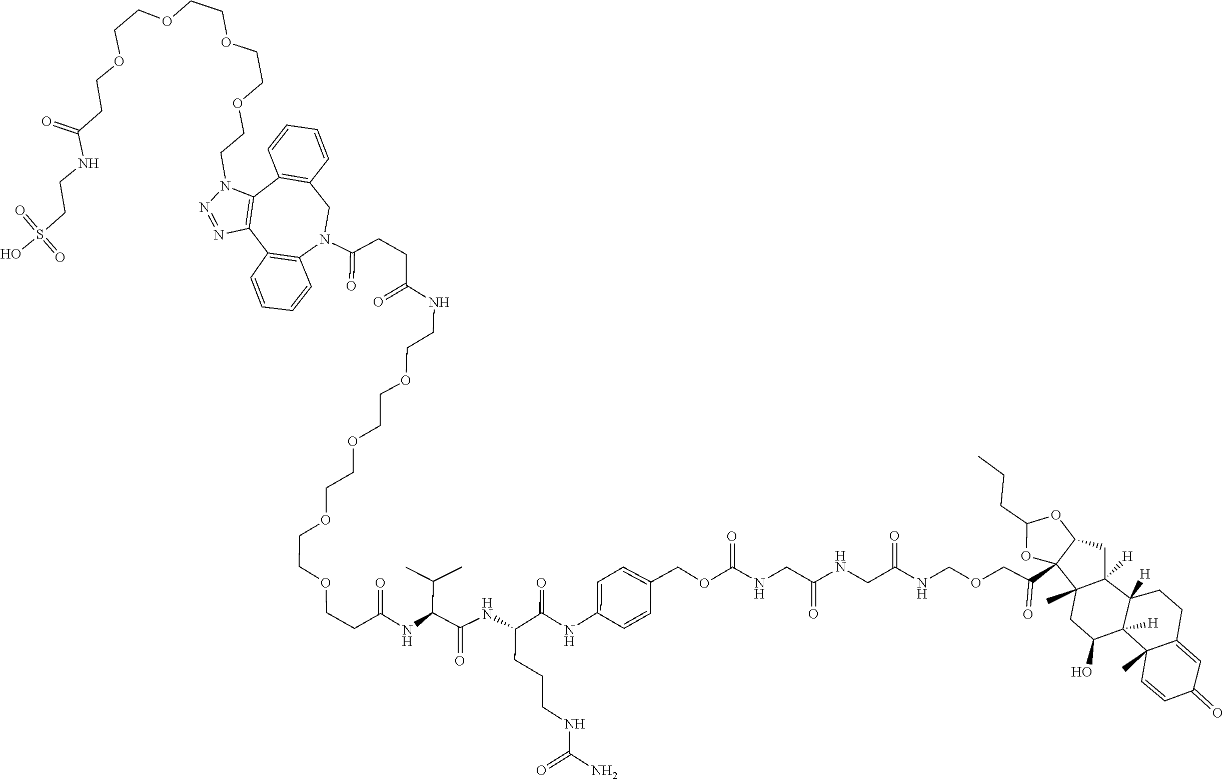 |
| Q1L1- PII-5 | 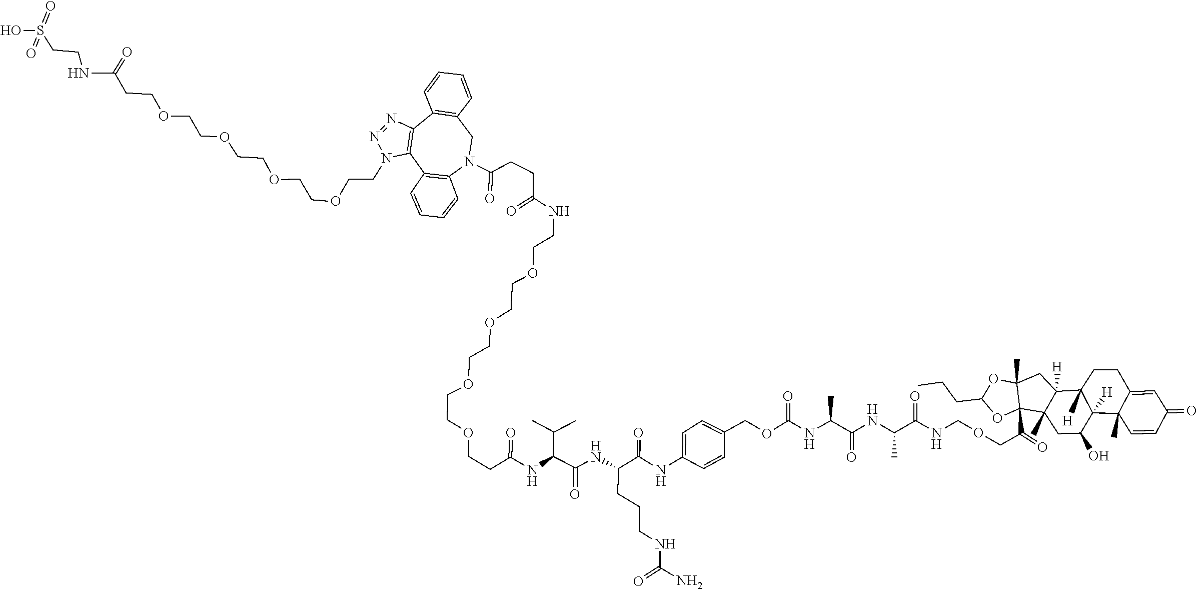 |
| | 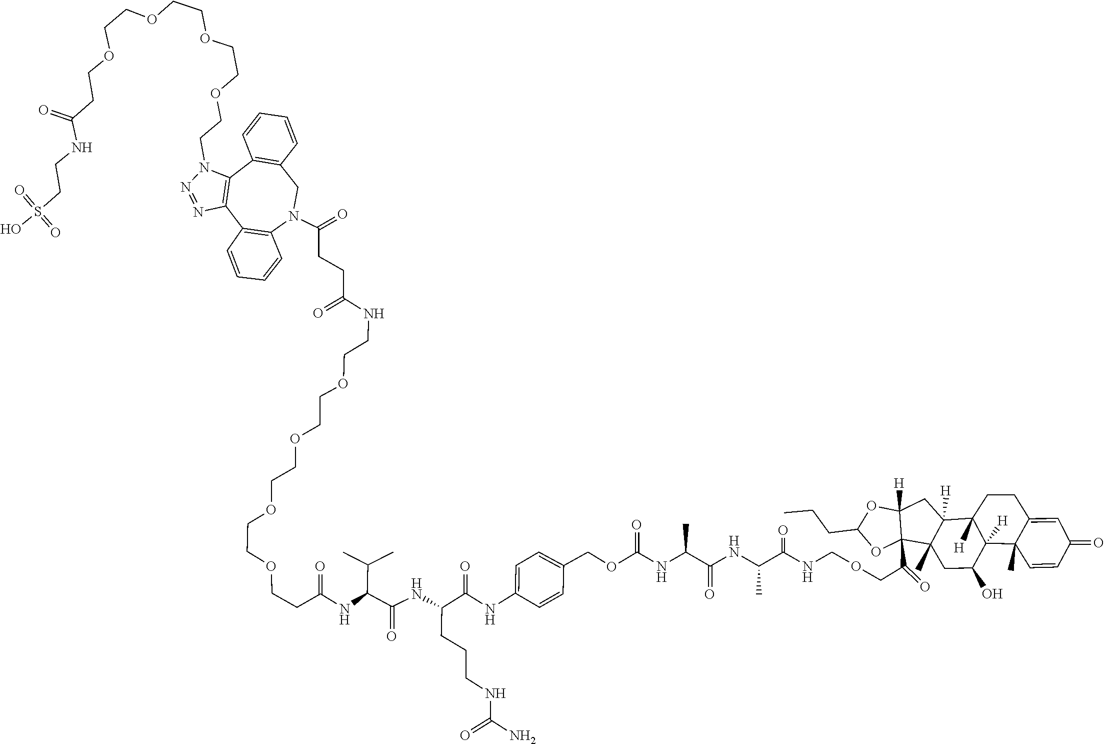 |
| Q1L1- PII-6 | 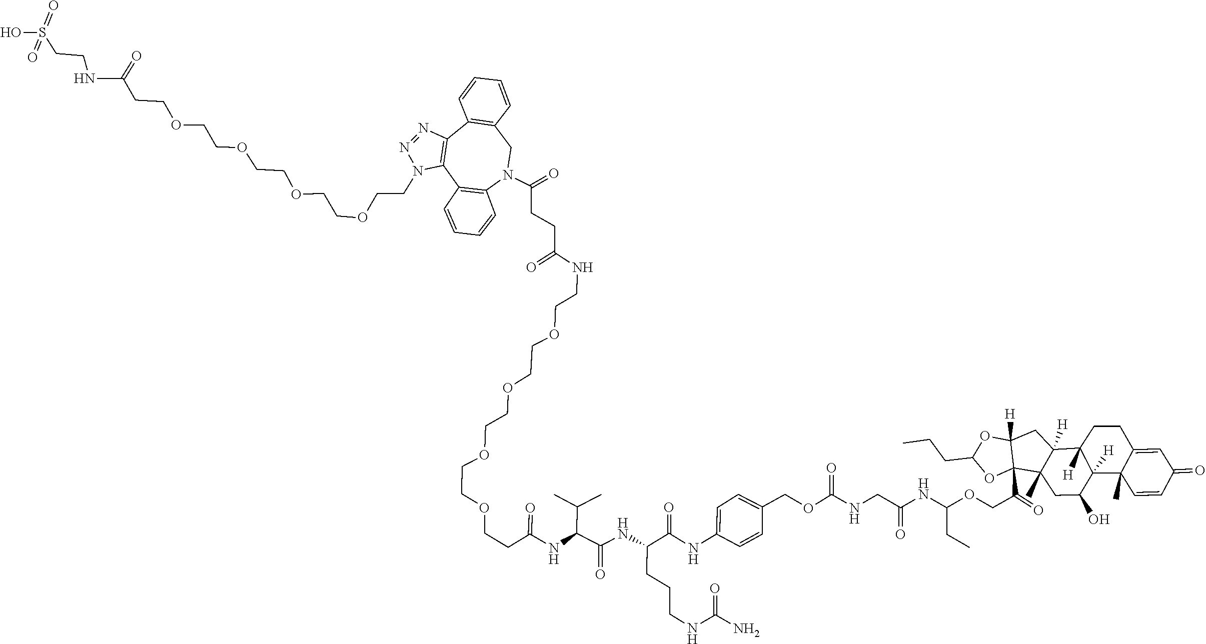 |
| | 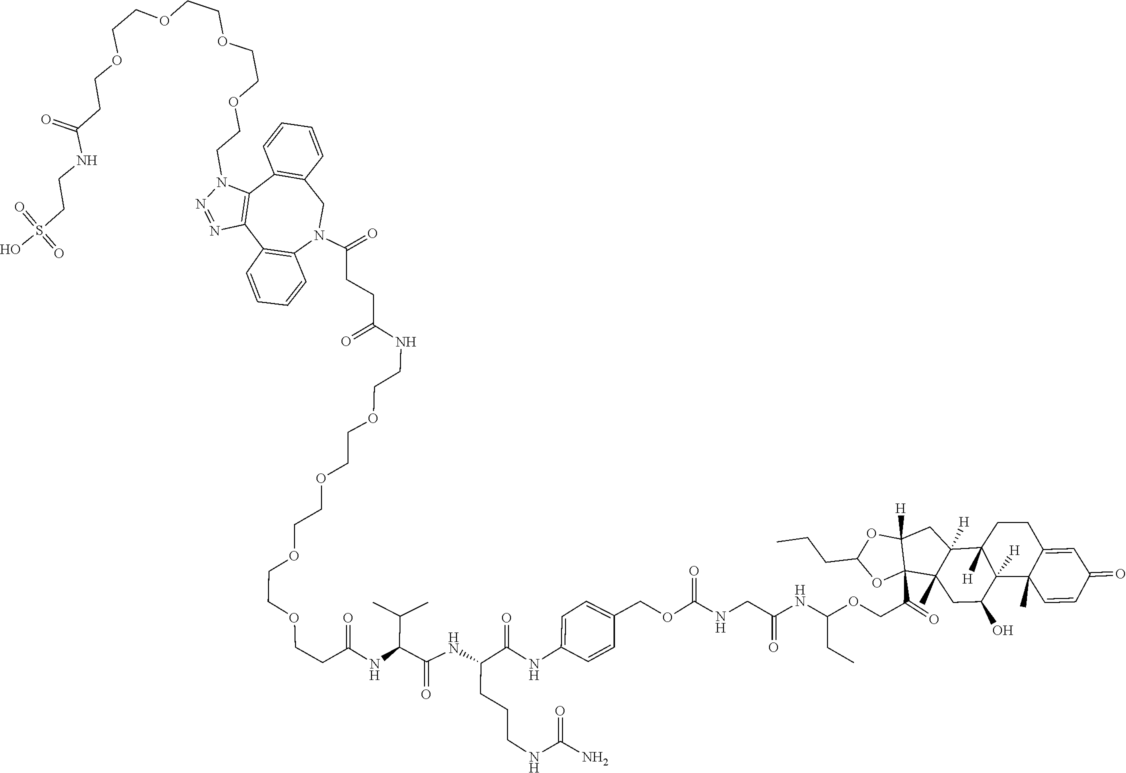 |
| Q1L1- PII-7 | 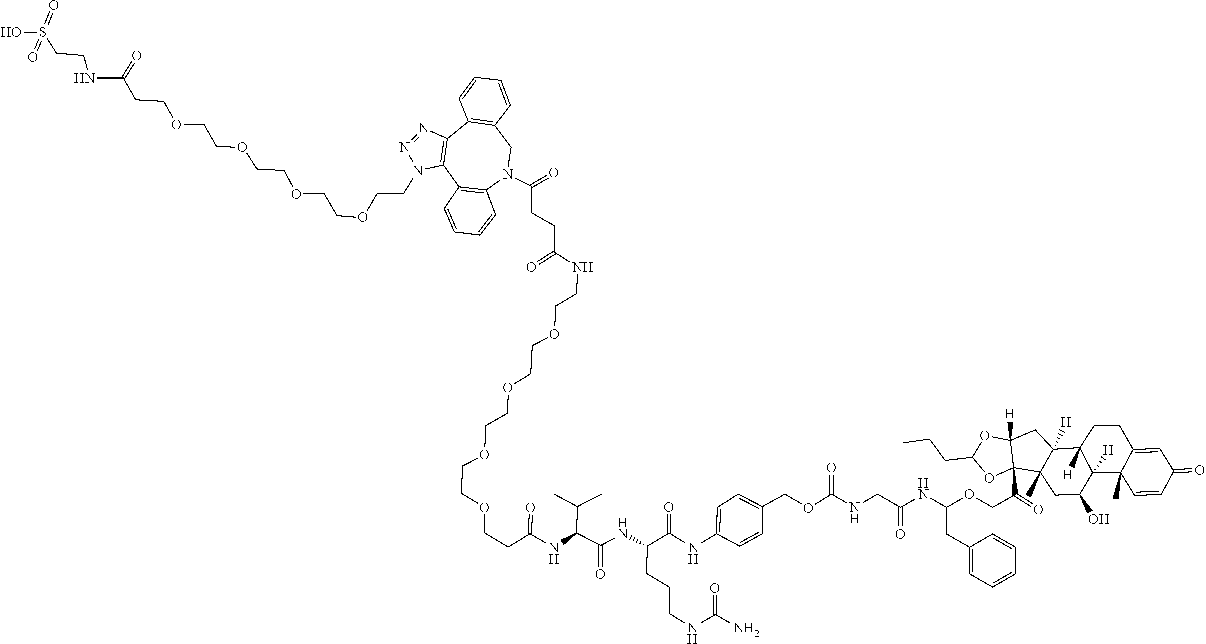 |
| | 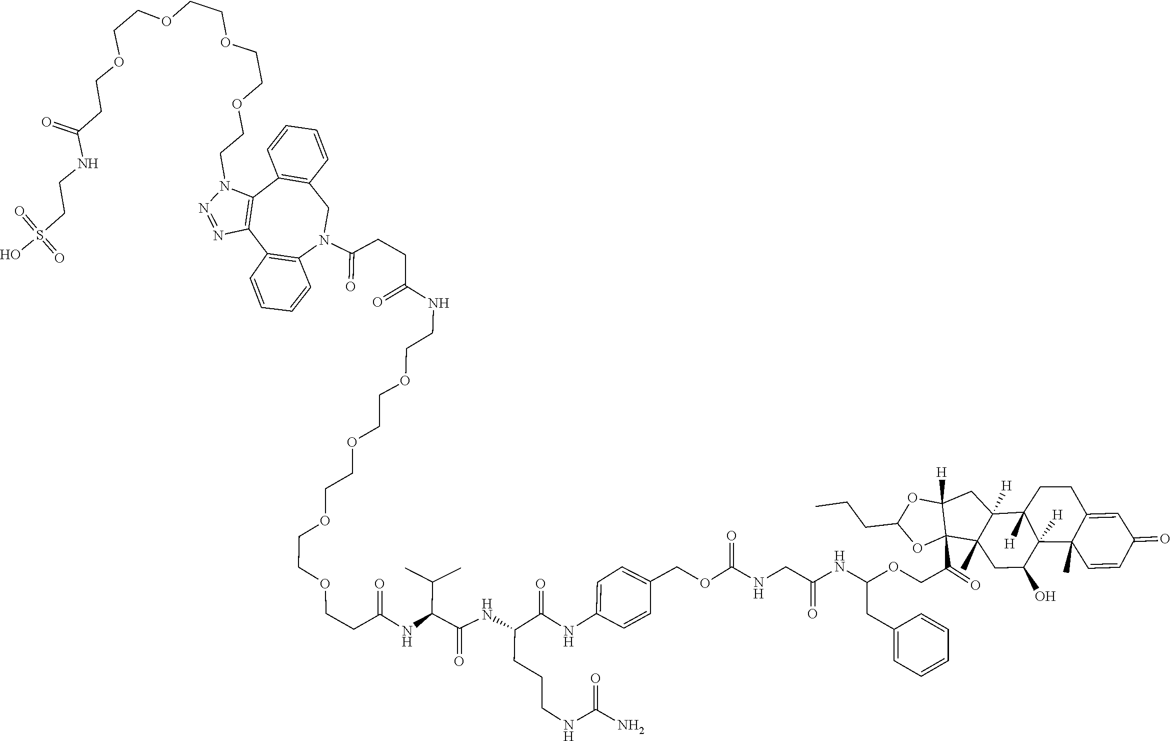 |
| Q1L1- PII-9 | 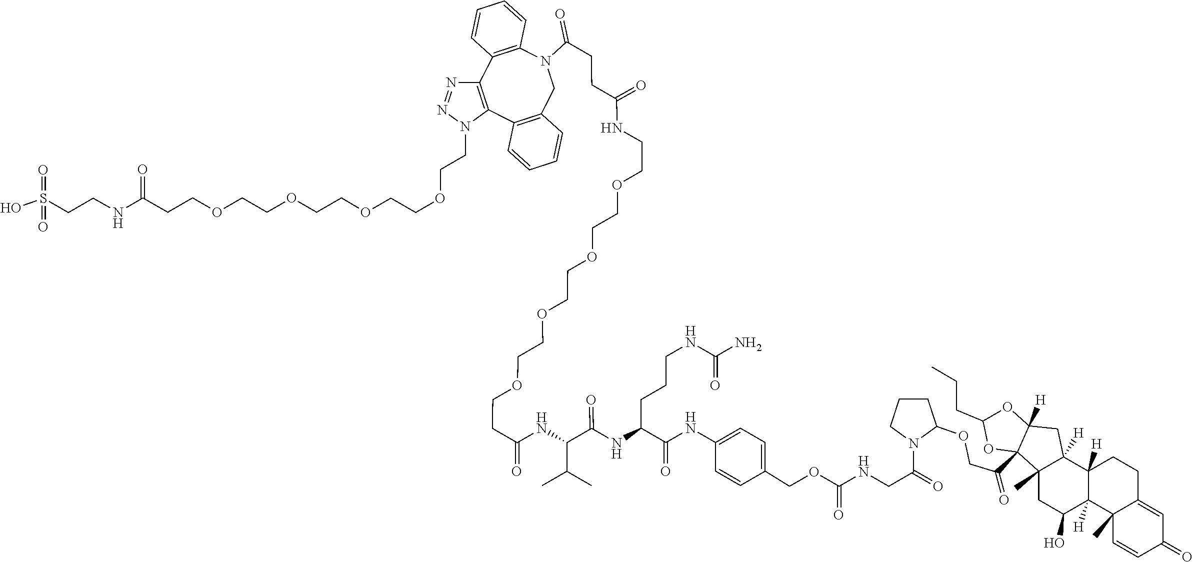 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4-c4c(nnn4CCOCCOCCOCCOCCC(=O)NCCS(=O)(=O)O)-c4ccccc43)C(C)C)cc2)O1] |
| | 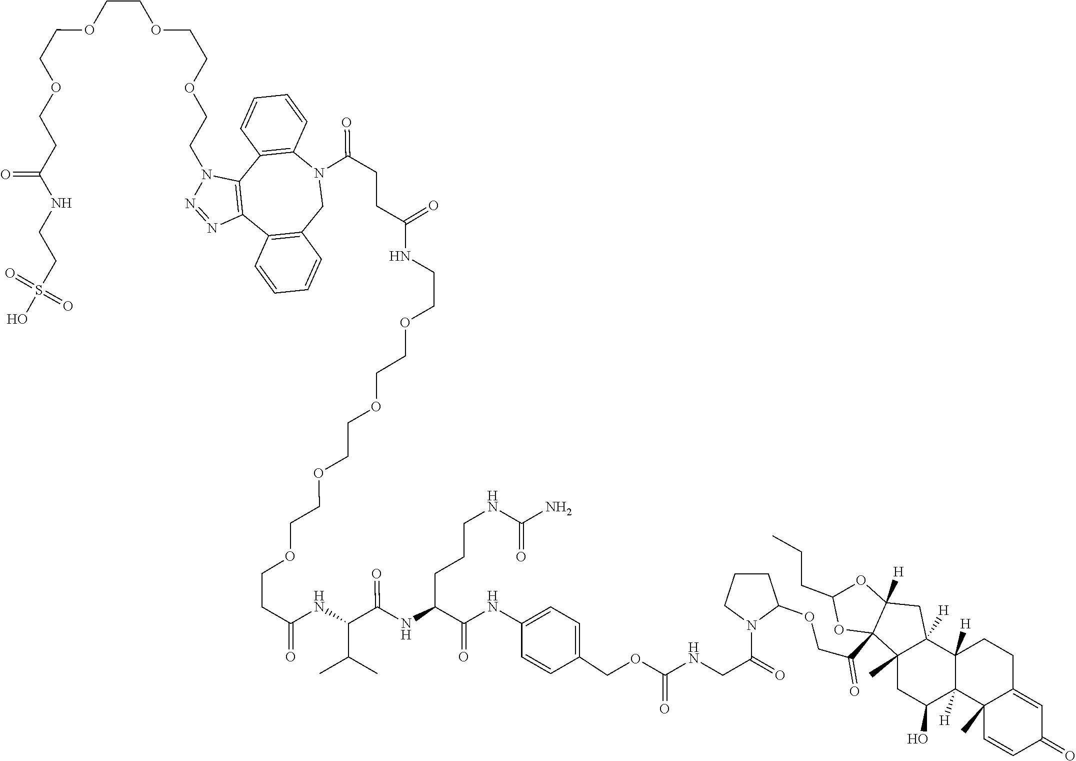 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4-c4nnn(CCOCCOCCOCCOCCC(=O)NCCS(=O)(=O)O)c4-c4ccccc43)C(C)C)cc2)O1] |
| Q1L1- PII-8 | 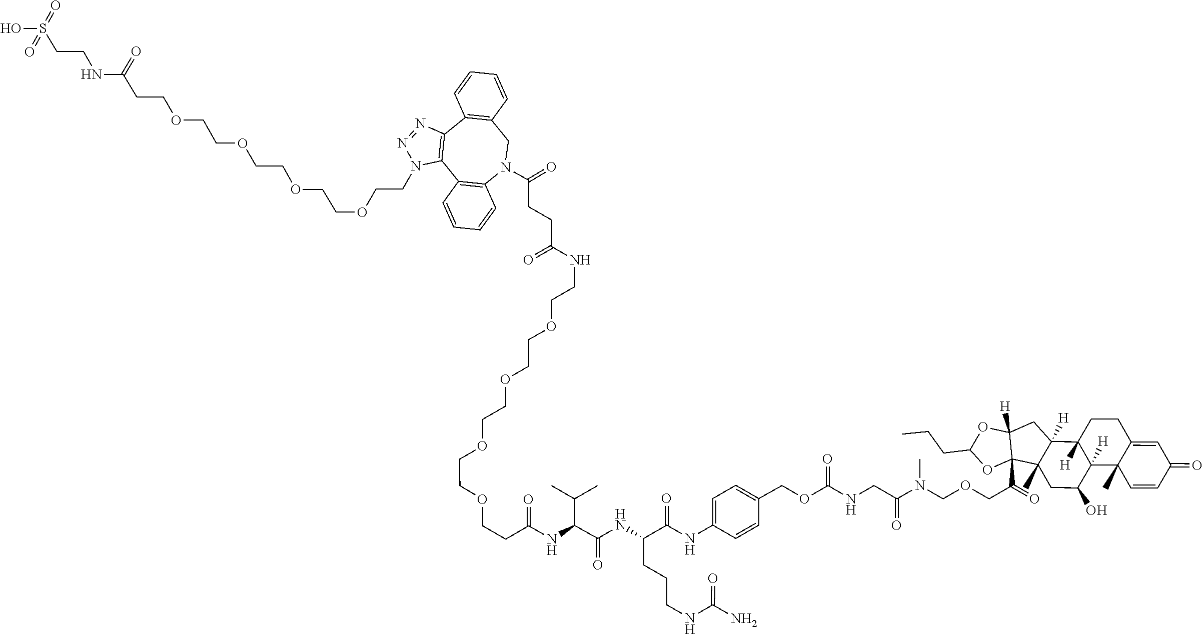 |
| | 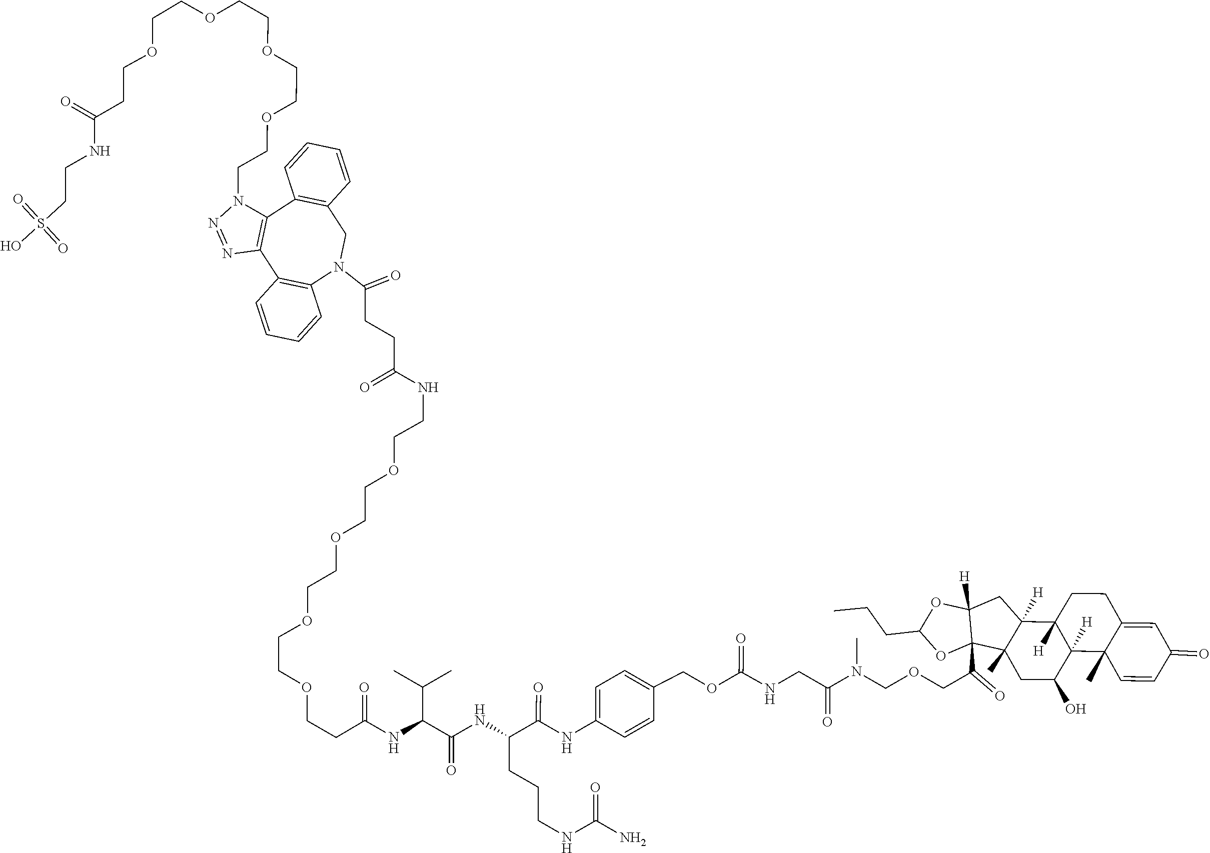 |
| Q1L1- PII-11 | 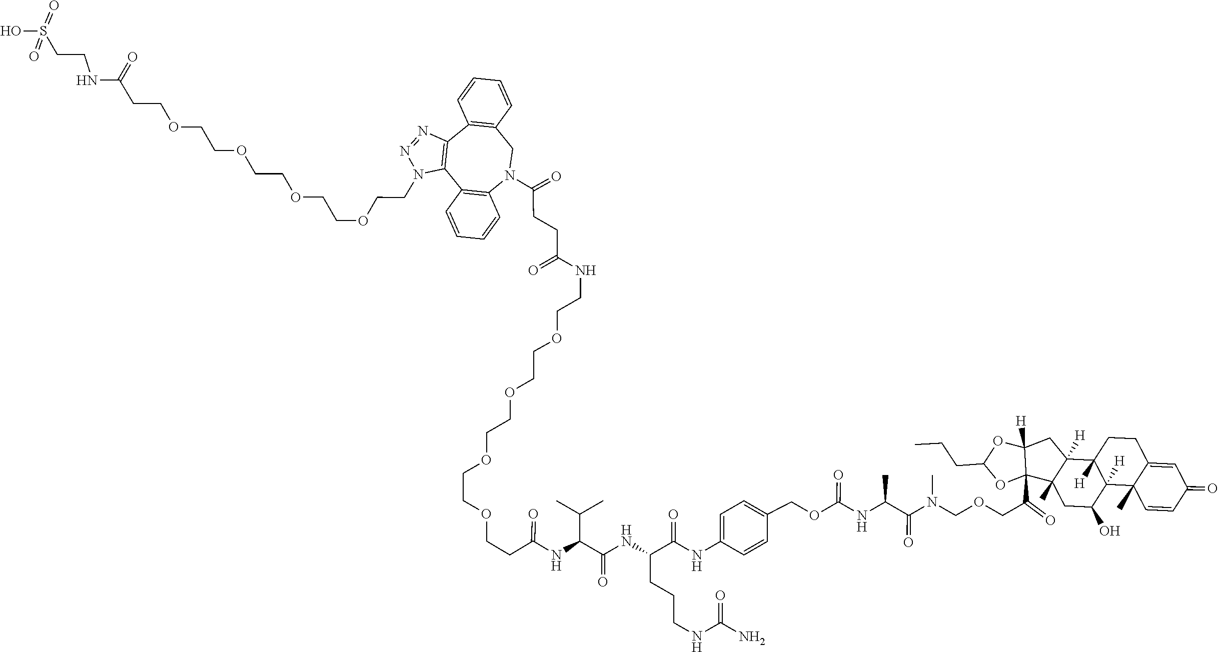 |
| | 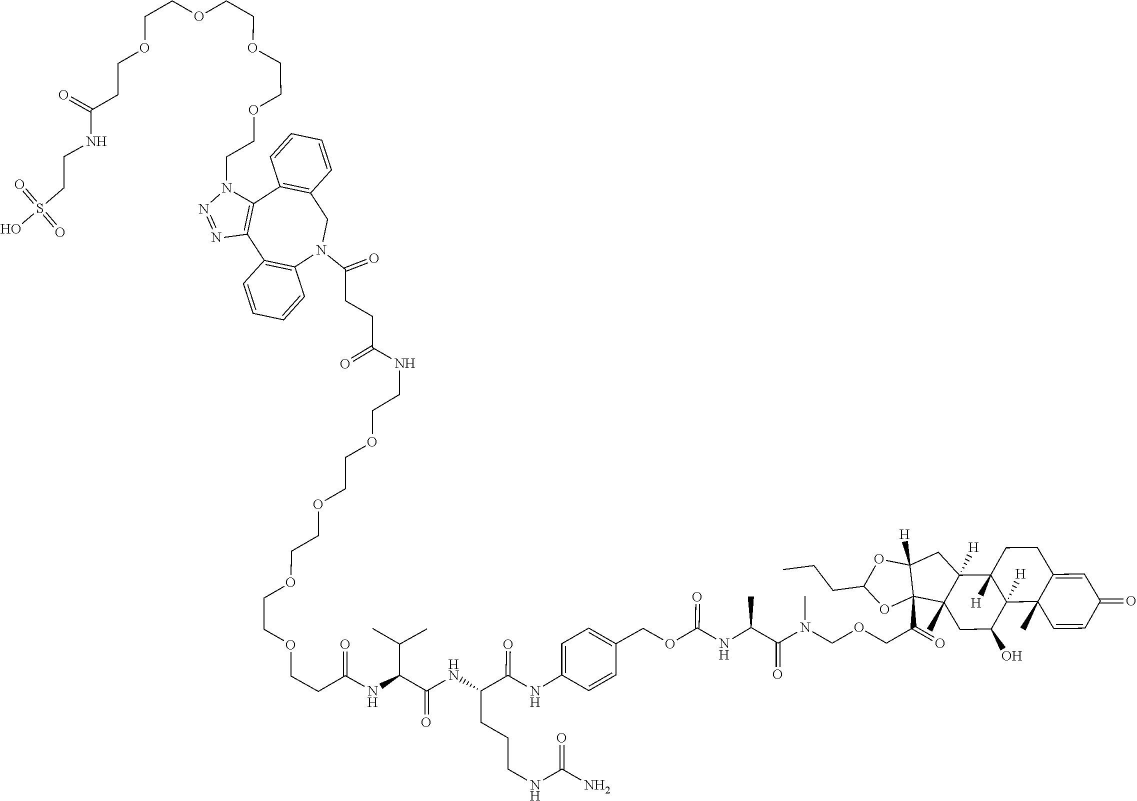 |
| Q1L1- PII-12 | 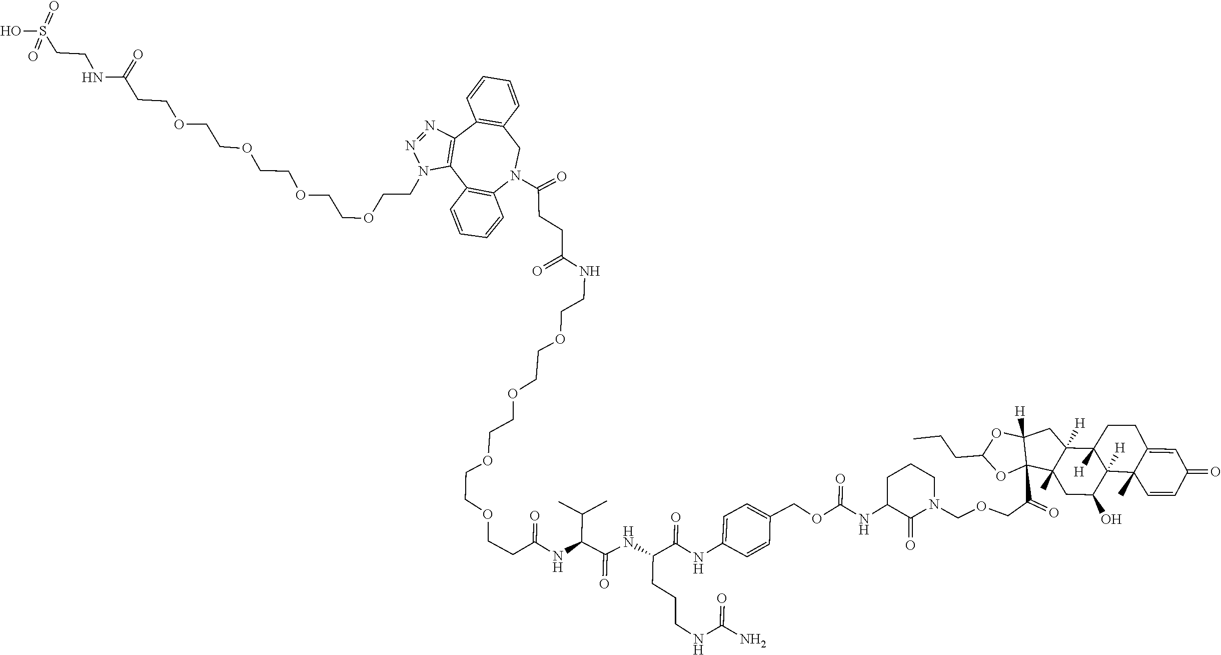 |
| | 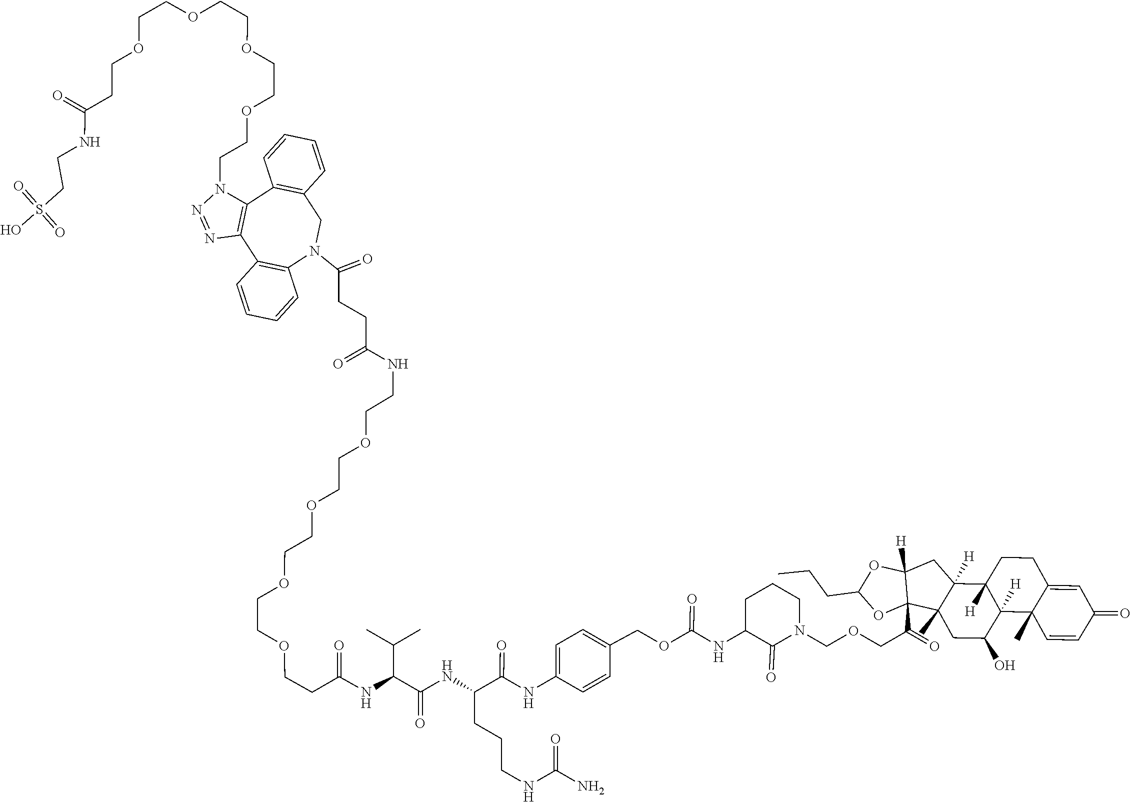 |
| Q1L6- PII-9 | 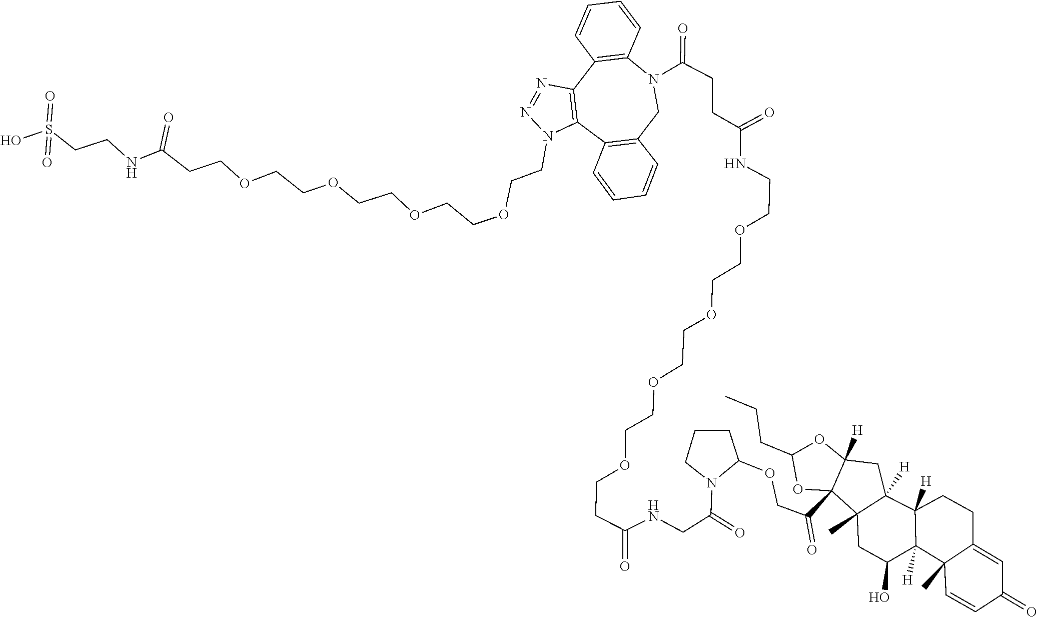 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CNC(=O)CCOCCOCCOCCOCCNC(=O)CCC(=O)N2Cc3ccccc3-c3c(nnn3CCOCCOCCOCCOCCC(=O)NCCS(=O)(=O)O)-c3ccccc32)O1] |
| | 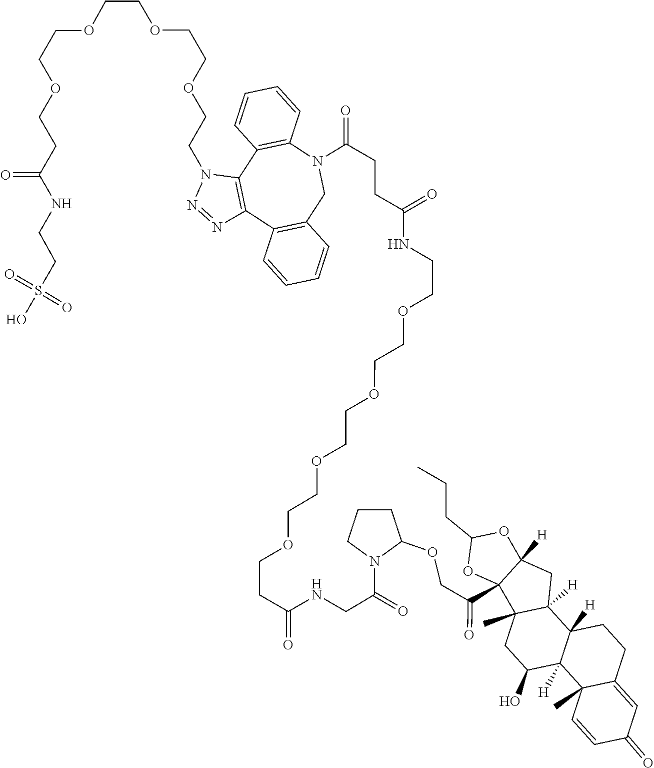 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CNC(=O)CCOCCOCCOCCOCCNC(=O)CCC(=O)N2Cc3ccccc3-c3nnn(CCOCCOCCOCCOCCC(=O)NCCS(=O)(=O)O)c3-c3ccccc32)O1] |
| Q2L6- PII-9 | 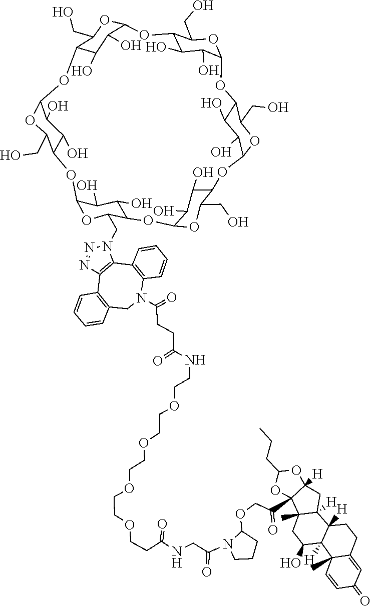 |
| | 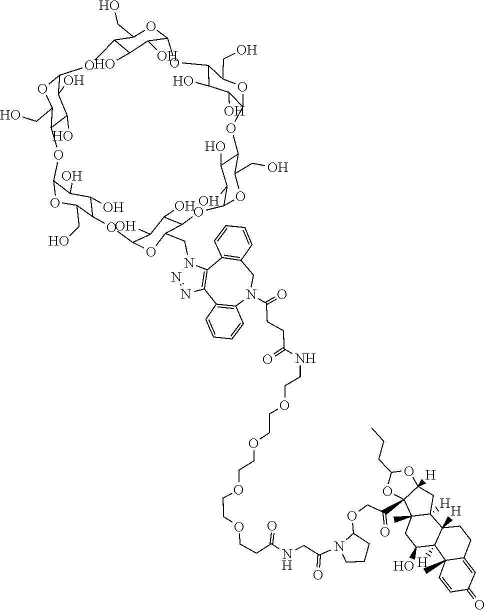 |
| Q1L7- PII-1 | 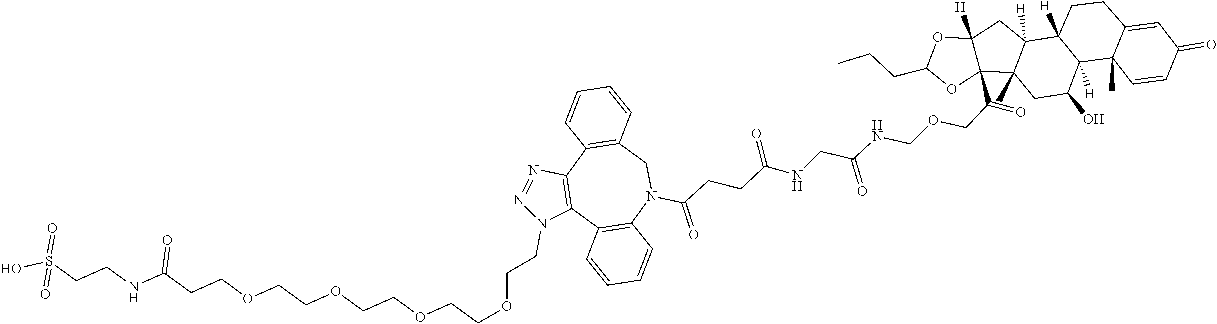 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CCC(=O)N2Cc3ccccc3-c3nnn(CCOCCOCCOCCOCCC(=O)NCCS(=O)(=O)O)c3-c3ccccc32)O1] |
| | 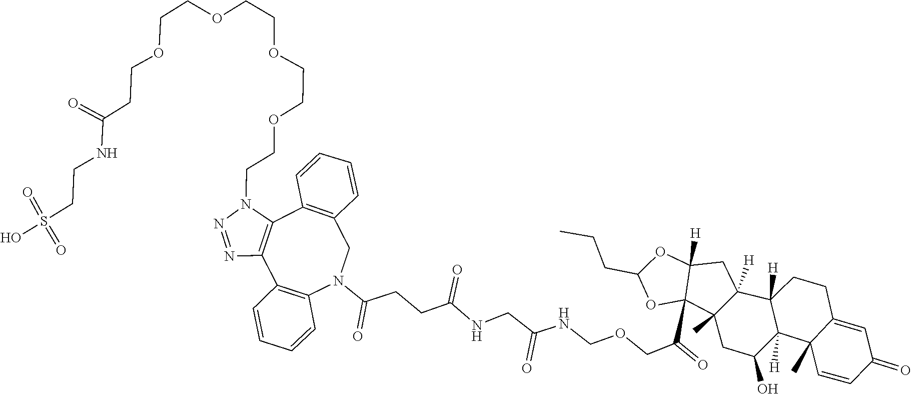 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CCC(=O)N2Cc3ccccc3-c3c(nnn3CCOCCOCCOCCOCCC(=O)NCCS(=O)(=O)O)-c3ccccc32)O1] |
| Q2L7- PII-1 | 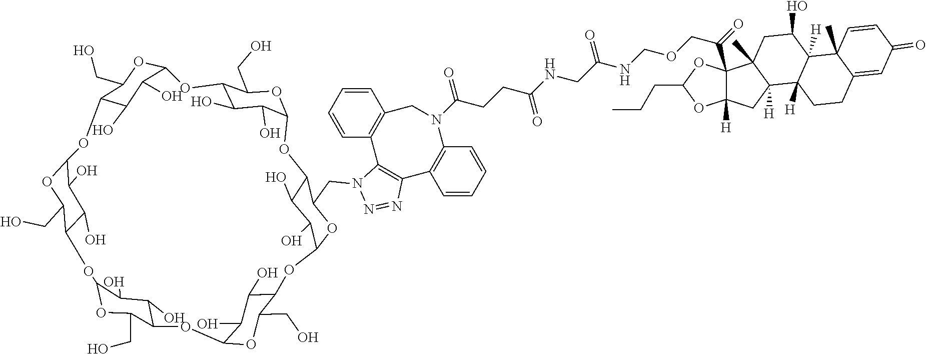 |
| | 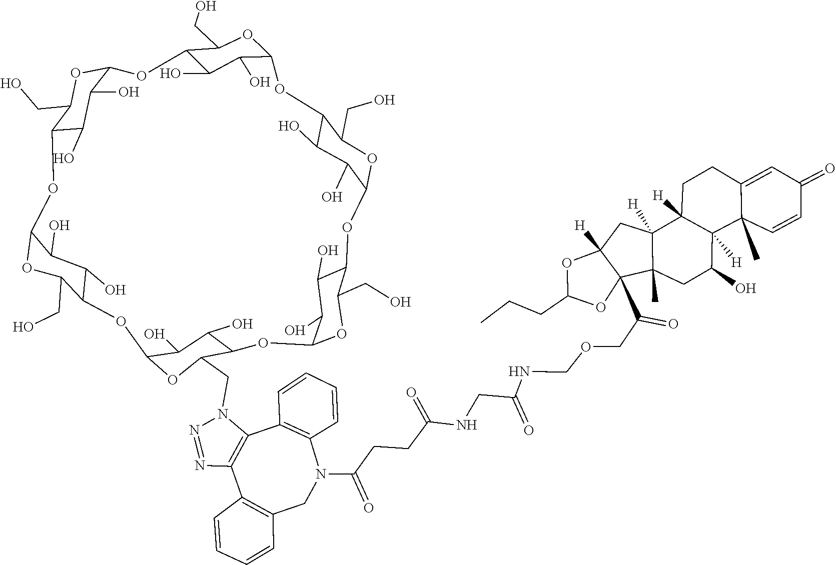 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CCC(=O)N2Cc3ccccc3-c3nnn(CC4OC5OC6C(CO)OC(OC7C(CO)OC(OC8C(CO)OC(OC9C(CO)OC(OC%10C(CO)OC(OC4C(O)C5O)C(O)C%10O)C(O)C9O)C(O)C8O)C(O)C7O)C(O)C6O)c3-c3ccccc32)O1] |
| Q1L15- PII-1 | 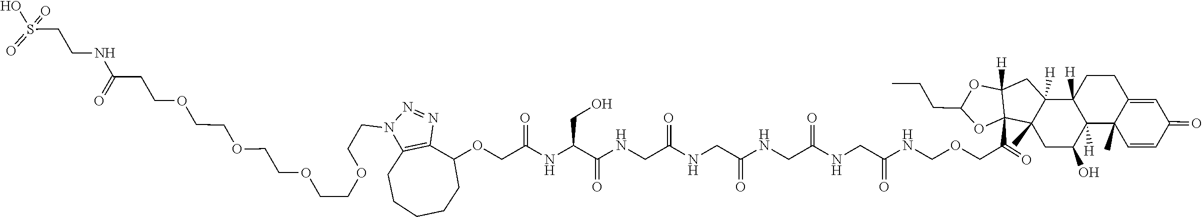 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CNC(=O)CNC(=O)CNC(=O)[C@H](CO)NC(=O)COC2CCCCCc3c2nnn3CCOCCOCCOCCOCCC(=O)NCCS(=O)(=O)O)O1] |
| | 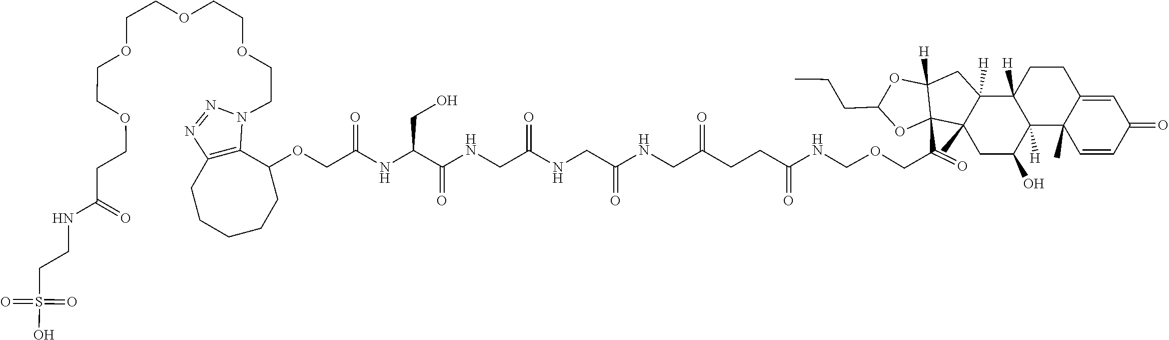 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CCC(=O)CNC(=O)CNC(=O)CNC(=O)[C@H](CO)NC(=O)COC2CCCCCc3nnn(CCOCCOCCOCCOCCC(=O)NCCS(=O)(=O)O)c32)O1] |
| Q1L17- PII-1 | 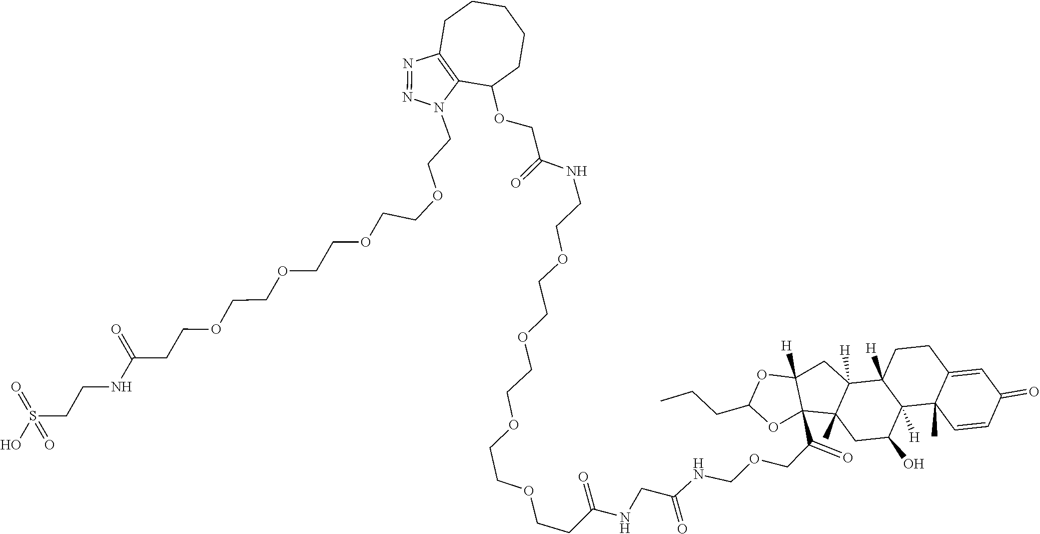 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CCOCCOCCOCCOCCNC(=O)COC2CCCCCc3nnn(CCOCCOCCOCCOCCC(=O)NCCS(=O)(=O)O)c32)O1] |
| | 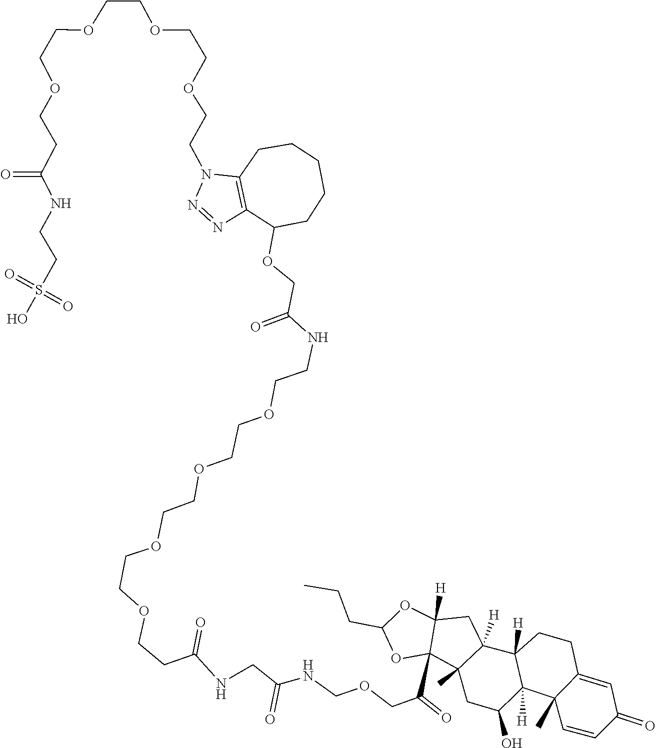 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CCOCCOCCOCCOCCNC(=O)COC2CCCCCc3c2nnn3CCOCCOCCOCCOCCC(=O)NCCS(=O)(=O)O)O1] |
| Q1L6- PII-13 | 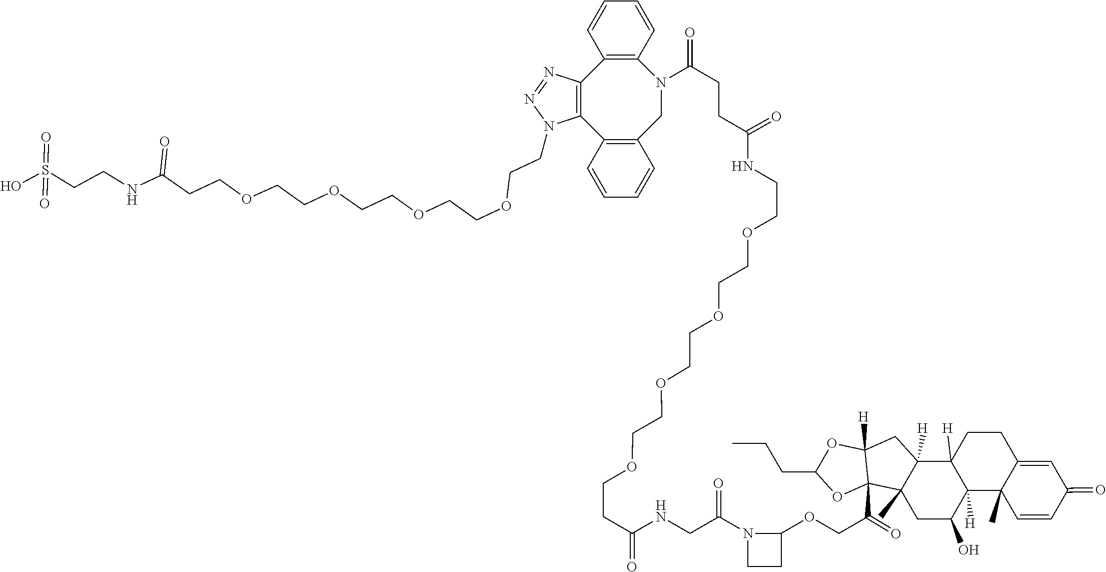 [SMILES: CCCC1O[C@@H]2C[C@H]3C4CCC5=CC(=O)C=C[C@]5(C)[C@H]4[C@@H](O)C[C@]3(C)[C@]2(C(=O)COC2CCN2C(=O)CNC(=O)CCOCCOCCOCCOCCNC(=O)CCC(=O)N2Cc3ccccc3-c3c(nnn3CCOCCOCCOCCOCCC(=O)NCCS(=O)(=O)O)-c3ccccc32)O1] |
| | 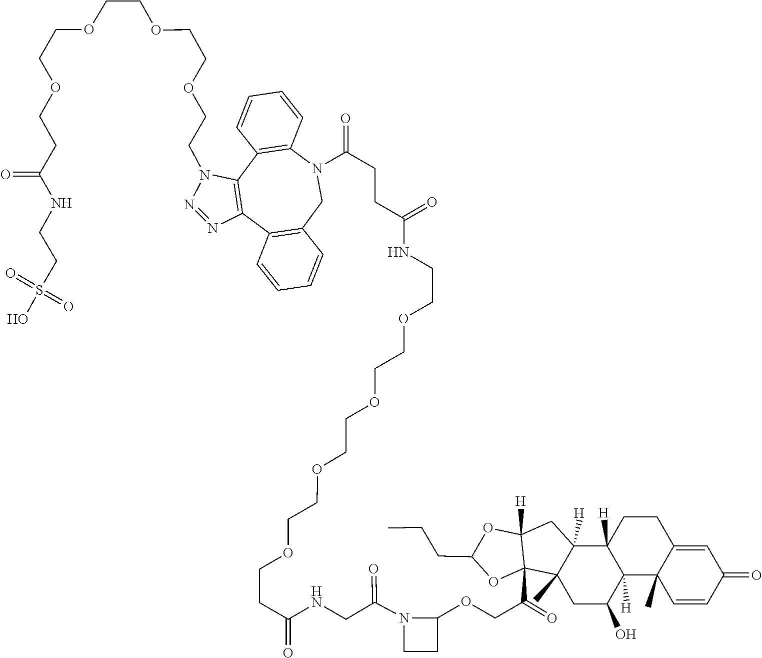 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCN2C(=O)CNC(=O)CCOCCOCCOCCOCCNC(=O)CCC(=O)N2Cc3ccccc3-c3nnn(CCOCCOCCOCCOCCC(=O)NCCS(=O)(=O)O)c3-c3ccccc32)O1] |
| Q1L13- PIII-1 | 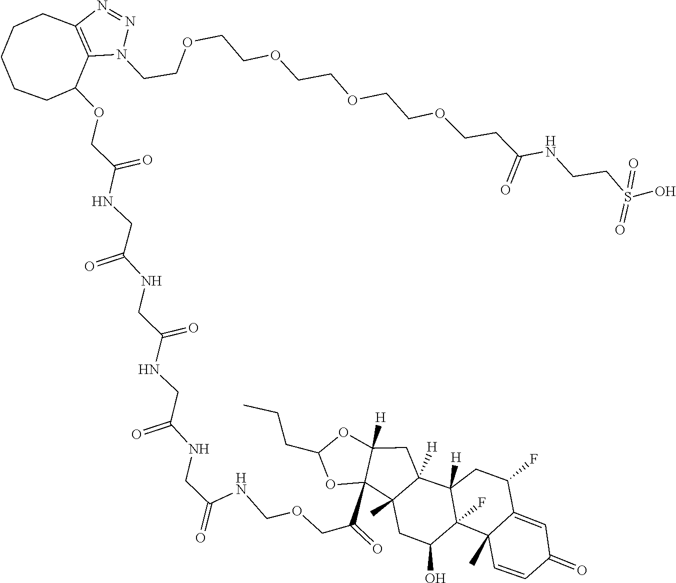 |
| | 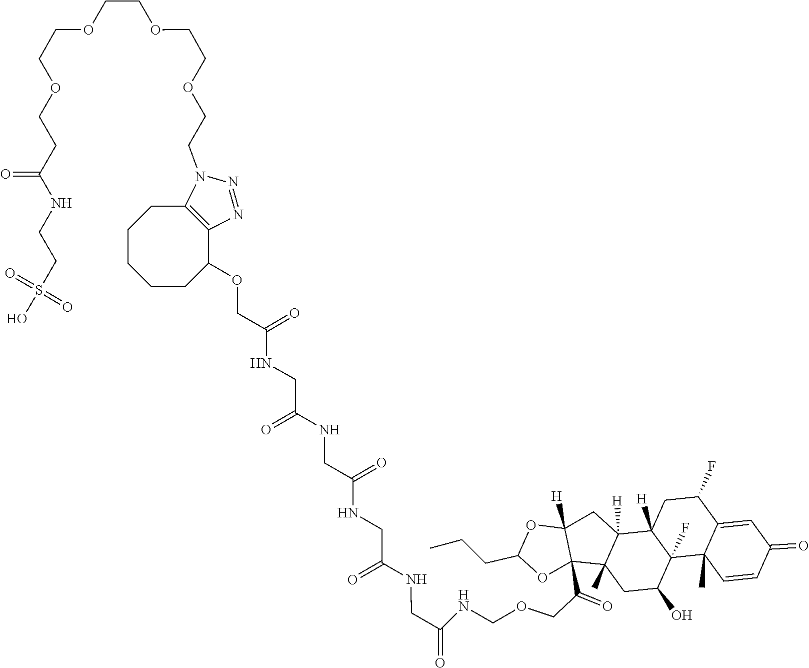 |
| Q1L1- PIV | 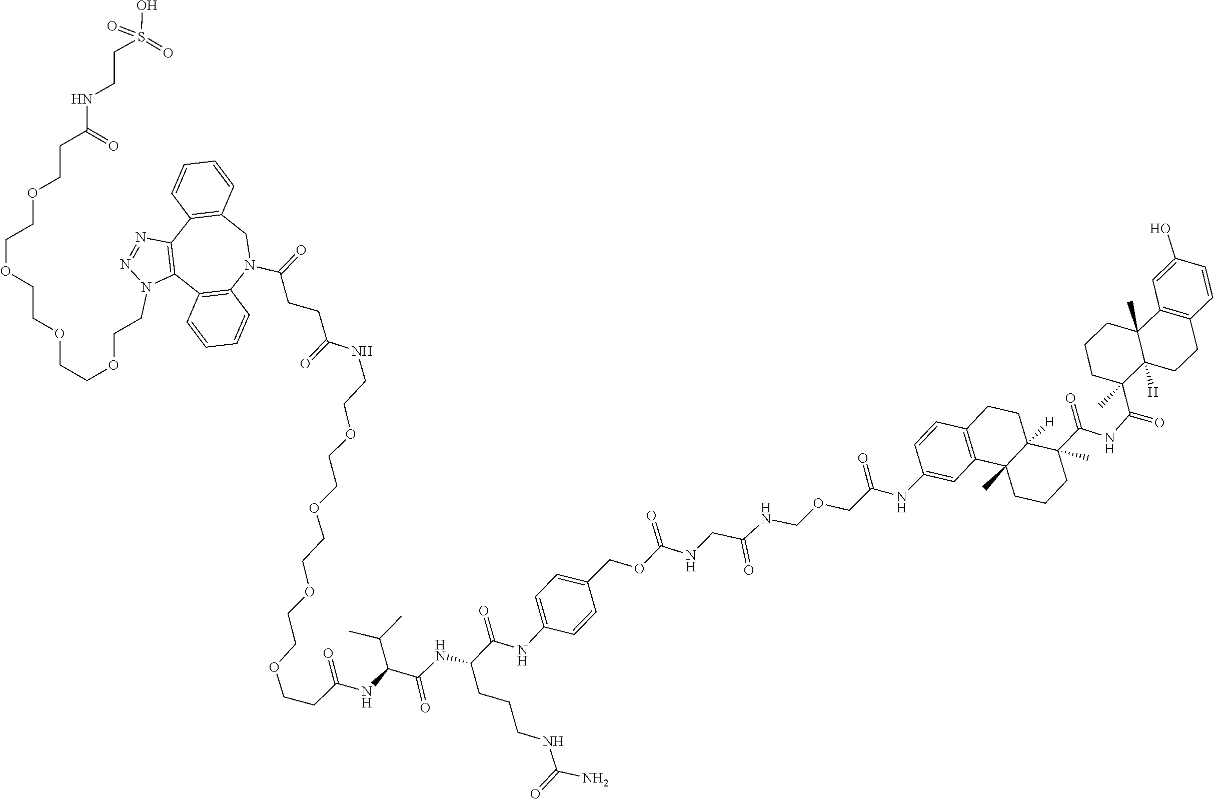 [SMILES: CC(C)[C@H](NC(=O)CCOCCOCCOCCOCCNC(=O)CCC(=O)N1Cc2ccccc2-c2nnn(CCOCCOCCOCCOCCC(=O)NCCS(=O)(=O)O)c2-c2ccccc21)C(=O)N[C@@H](CCCNC(N)=O)C(=O)Nc1ccc(COC(=O)NCC(=O)NCOCC(=O)Nc2ccc3c(c2)[C@@]2(C)CCC[C@](C)(C(=O)NC(=O)[C@@]4(C)CCC[C@]5(C)c6cc(O)ccc6CC[C@@H]45)[C@@H]2CC3)cc1] |
| | 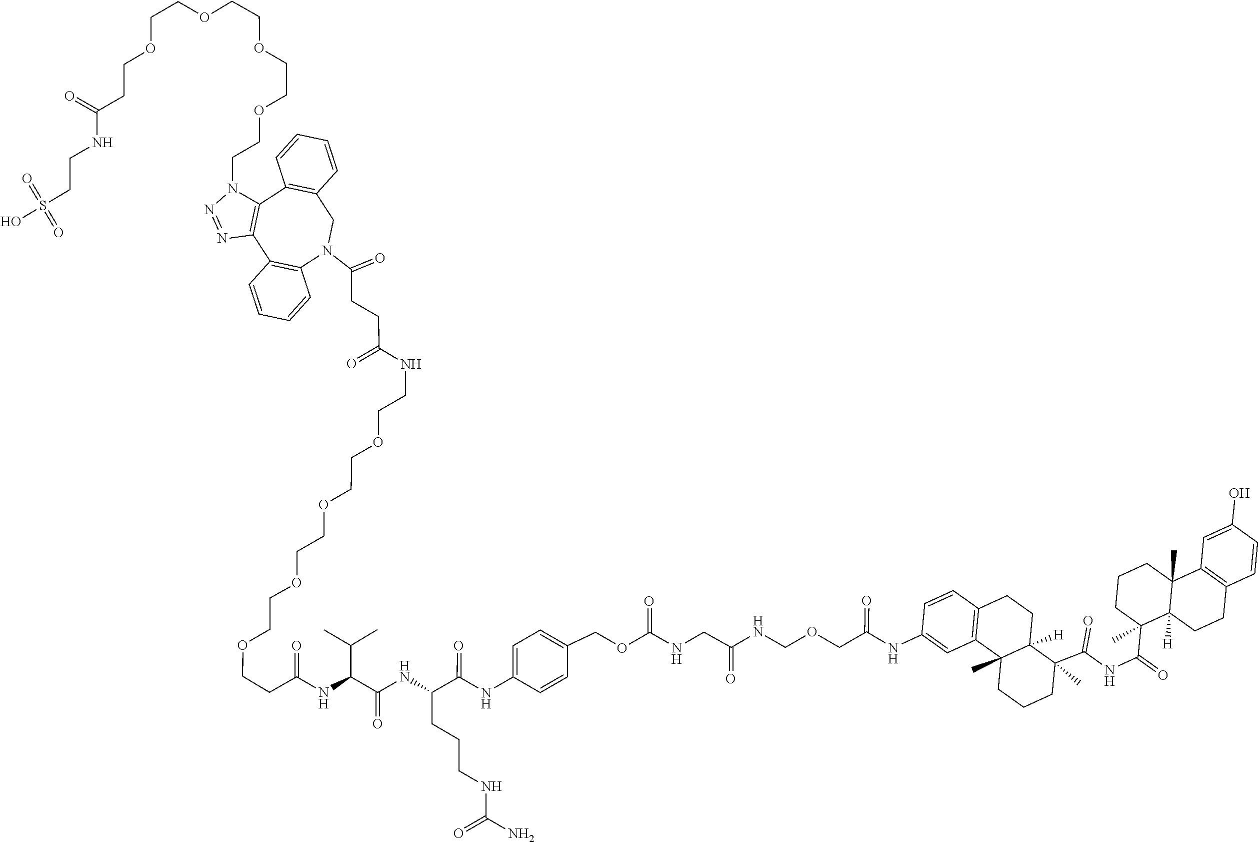 [SMILES: CC(C)[C@H](NC(=O)CCOCCOCCOCCOCCNC(=O)CCC(=O)N1Cc2ccccc2-c2c(nnn2CCOCCOCCOCCOCCC(=O)NCCS(=O)(=O)O)-c2ccccc21)C(=O)N[C@@H](CCCNC(N)=O)C(=O)Nc1ccc(COC(=O)NCC(=O)NCOCC(=O)Nc2ccc3c(c2)[C@@]2(C)CCC[C@](C)(C(=O)NC(=O)[C@@]4(C)CCC[C@]5(C)c6cc(O)ccc6CC[C@@H]45)[C@@H]2CC3)cc1] |

TABLE 6a

| Quenched | | | | | Solubility |
|---|---|---|---|---|---|
| linker-payloads | Azides | Linker-payloads | HPLC Rₜ (min) | MS m/z | (mg/mL) |
| Q1L1-PI | taurine- | Taurine-PEG4- | 1.74 (B) | 712.8 | >1 |
| | PEG4-azide | TDIBAC-PEG₄- | [LCMS] | [(M − Bud)/2 + H] | |
| | | vcPAB-G-NHCH₂- | | | |
| | | Dex | | | |
| Q1L1-PII-1 | taurine- | Taurine-PEG4- | 6.76 | 619.0 | |
| | PEG4-azide | TDIBAC-PEG₄- | | (M/3 + H) | |
| | | vcPAB-G-NHCH₂- | | | |
| | | Bud | | | |
| Q2L1-PII-1 | CD-N₃ | DIBAC-suc-PEG4- | 6.76 (B) | 819.0 | >1 |
| | | vcPAB-PII-1 | | (M/3 + H) | |
| Q1L1-PII-2 | taurine- | Taurine-PEG4- | 6.83 | 623.8 | |
| | PEG4-azide | TDIBAC-PEG₄- | | (M/3 + H), | |
| | | vcPAB-A-NHCH₂- | | 720.0 | |
| | | Bud | | [(M − M_{Bud})/2 + H] | |
| Q1L1-PII-3 | taurine- | Taurine-PEG4- | 7.21 | 757.8 | |
| | PEG4-azide | TDIBAC-PEG₄- | | [(M − M_{Bud})/2 + H] | |
| | | vcPAB-F-NHCH₂- | | | |
| | | Bud | | | |
| Q1L1-PII-4 | taurine- | Taurine-PEG4- | 6.65 | 741.5 | |
| | PEG4-azide | TDIBAC-PEG₄- | | [(M − M_{Bud})/2 + H] | |
| | | vcPAB-GG- | | | |
| | | NHCH₂-Bud | | | |
| Q1L1-PII-5 | taurine- | Taurine-PEG4- | 6.82 | 755.5 | |
| | PEG4-azide | TDIBAC-PEG₄- | | [(M − M_{Bud})/2 + H] | |
| | | vcPAB-AA- | | | |
| | | NHCH₂-Bud | | | |
| Q1L1-PII-6 | taurine- | Taurine-PEG4- | 6.99, 7.05 | 726.7 | >1 |
| | PEG4-azide | TDIBAC-PEG₄- | (B) | [(M − Bud)/2 + H] | |
| | | vcPAB-G- | | | |
| | | NHCHEt-Bud | | | |
| Q1L1-PII-7 | taurine- | Taurine-PEG4- | 8.21, 8.27 | 648.0 | >1 |
| | PEG4-azide | TDIBAC-PEG₄- | (B) | (M/3 + H), | |
| | | vcPAB-G- | | 758.0 | |
| | | NHCHBn-Bud | | [(M − Bud)/2 + H] | |
| Q1L1-PII-8 | taurine- | Taurine-PEG4- | 6.92 (B) | 935.0 | >1 |
| | PEG4-azide | TDIBAC-PEG₄- | | (M/2 + H), | |
| | | vcPAB-G- | | 720.0 | |
| | | NMeCH₂-Bud | | [(M − Bud)/2 + H] | |
| Q1L1-PII-11 | taurine- | Taurine-PEG4- | 6.94 | 726.4 | |
| | PEG4-azide | TDIBAC-PEG₄- | | [(M − M_{Bud})/2 + H] | |
| | | vcPAB-A- | | | |
| | | NMeCH₂-Bud | | | |
| Q1L1-PII-12 | taurine- | Taurine-PEG4- | 1.76 | 947.8 | |
| | PEG4-azide | TDIBAC-PEG₄- | (LCMS) | (M/2 + H) | |
| | | vcPAB-amino- | | | |
| | | piperidinone-CH₂- | | | |
| | | Bud | | | |
| Q1L1-PII-9 | taurine- | Taurine-PEG4- | 7.03 (B) | 732.8 | >1 |
| | PEG4-azide | TDIBAC-PEG4- | | [(M − Bud)/2 + H] | |
| | | vcPAB-G- | | | |
| | | pyrrolidine-Bud | | | |
| Q1L7-PII-1 | taurine- | Taurine-PEG4- | 6.74 | 901.5 | |
| | PEG4-azide | TDIBAC-G- | | (M/2 + H) | |
| | | NHCH₂-Bud | | | |
| Q1L15-PII-1 | taurine- | Taurine-PEG4- | 1.68 | 907.1 | |
| | PEG4-azide | TCOT-SGGGG- | (LCMS) | (M − M_{Bud})/2 + H] | |
| | | NHCH₂-Bud | | | |
| Q1L17-PII-1 | taurine- | Taurine-PEG4- | 6.55 | 896.2 | |
| | PEG4-azide | TCOT-PEG₄-G- | | (M − M_{Bud} + H) | |
| | | NHCH₂-Bud | | | |
| Q1L6-PII-9 | taurine- | Taurine-PEG4- | 7.04 | 1060.4 | |
| | PEG4-azide | TDIBAC-PEG₄-G- | | (M − M_{Bud} + H) | |
| | | Pyrrolidine-Bud | | | |
| Q2L6-PII-9 | CD-N₃ | DIBAC-suc-PEG4- | 6.94 (B) | 1067.6 | >1 |
| | | PII-9 | | (M/2 + Na), | |
| Q1L6-PII-13 | taurine- | Taurine-PEG4- | 7.97 | 738.5 | |
| | PEG4-azide | TDIBAC-PEG₄-G- | | (M/2 + H) | |
| | | Azetidine-Bud | | | |
| Q2L7-PII-1 | CD-N₃ | DIBAC-suc-PII-1 | 6.74 (B) | 901.7 | >1 |
| | | | | (M/2 + H) | |
| Q1L13-PIII-1 | taurine- | Taurine-PEG4- | 6.49 | 820.2 | |
| | PEG4-azide | TCOT-GGGG- | | (M − M_{III} + H) | |
| | | NHCH₂-III | | | |
| Q1L1-PIV | taurine- | Taurine-PEG4- | 6.88 | 712.9 | |
| | PEG4-azide | TDIBAC-PEG4- | | [(M − M_{IV})/2 + H] | |
| | | vcPAB-G-NHCH₂-IV | | | |

### Synthesis of Q2L1-PII-1

To a solution of L1-PII-1 (5 mg, 3.4 μmol) in DMF (1.0 mL) was added compound CD-N₃ (7.0 mg, 6.8 μmol). The reaction mixture was stirred at RT overnight, which was monitored by LCMS. The reaction mixture was directly purified by prep-HPLC to give compound Q2L1-PII-1 (5 mg, 60% yield) as a white solid. ESI m/z: 819.0 (M/3+H)⁺.

### pH Stability of Linker-Payloads in Buffers

As the solubility of the linker-payloads were poor (<0.02 mg/mL aq. DMSO (20%)), the pH stability of these compounds was not tested. The quenched linker-payloads were water soluble and used for the pH stability test.

### Procedures to Test pH Stability of Quenched Linker-Payloads

To a solution of compound (payloads or quenched-linker-payloads) in DMSO (0.2-1.0 mg/mL) was added corresponding PBS buffer (V(buffer)/V(DMSO)=5) to give test samples (pH 3, 4, 5, 6, 7.4, and 8.5, or other special pH). The test samples were incubated at RT for at least 3 days and monitored by LCMS (i.e., at 1 hr, 2 hr, 4 hr, 8 hr, and 1 day, 2 day, 3 day, 4 day, 5 day, 6 day, 7 day, 8 day, 9 day, and 21 day time points). Initial concentrations of the samples were 0.2 mg/mL (DMSO/buffer, v/v=1/5). Percentages of the remaining compounds at the corresponding times were reported; all compounds were stable at pH 7.4 and pH 8.5 at Day 7 with no Budesonide released, and other impurities were less than 3% (data not shown in Table 7). The payloads and quenched linker-payloads were mostly stable at pH >5.0, 6.0, 7.4 and 8.5 after 3 days. In more acidic conditions, such as pH 1.0, the payloads or linker-payloads were easily cleaved to release D*-OH. Some quenched linker-payloads were cleaved to release Budesonide at pH 4.0 and pH 3.0 after 24 hours. Results are shown in Tables 7, 7a, 7b, 7c, and 7d.

TABLE 7
pH Stability of Quenched Linker-Payloads
for Releasing Parent Payloads

| Cpd# | Time | | DMSO | pH 3.0 | pH 4.0 | pH 5.0 | pH 7.4 |
|---|---|---|---|---|---|---|---|
| Q1L1-PI | 1 | hr | 100 | 100 | 100 | 100 | 100 |
| | 4 | hr | 100 | 100 | 100 | 100 | 100 |
| | 1 | Day | 100 | 100 | 100 | 100 | 100 |
| | 2 | Day | 100 | 100 | 100 | 100 | 100 |
| | 3 | Day | 100 | 96 | 100 | 100 | 100 |
| | 4 | Day | 100 | 95 | 100 | 100 | 100 |
| | 7 | Day | 100 | 91 | 100 | 100 | 100 |
| Q1L1-PII-1 | 1 | hr | 100 | 100 | 100 | 100 | 100 |
| | 4 | hr | 100 | 100 | 100 | 100 | 100 |
| | 1 | Day | 100 | 100 | 100 | 100 | 100 |
| | 2 | Day | 100 | 100 | 100 | 100 | 100 |
| | 3 | Day | 100 | 100 | 100 | 100 | 100 |
| | 4 | Day | 100 | 100 | 100 | 100 | 100 |
| | 7 | Day | 100 | 100 | 100 | 100 | 100 |
| Q1L1-PII-6 | 1 | hr | 100 | 73 | 97 | 100 | 100 |
| | 4 | hr | 100 | 54 | 93 | 100 | 100 |
| | 1 | Day | 100 | 14 | 84 | 100 | 100 |
| | 2 | Day | 100 | 5 | 61 | 98 | 100 |
| | 3 | Day | 100 | 3 | 46 | 97 | 100 |
| | 4 | Day | 100 | | 37 | 92 | 100 |
| | 7 | Day | 100 | | 37 | 90 | 100 |
| Q1L1-PII-7 | 1 | hr | 100 | 98 | 100 | 100 | 100 |
| | 4 | hr | 100 | 93 | 100 | 100 | 100 |
| | 1 | Day | 100 | 76 | 100 | 100 | 100 |
| | 2 | Day | 100 | 59 | 94 | 100 | 100 |
| | 3 | Day | 100 | 45 | 91 | 100 | 100 |
| | 4 | Day | 100 | 35 | 88 | 100 | 100 |
| | 7 | Day | 100 | 19 | 82 | 100 | 100 |
| Q1L1-PII-8 | 1 | hr | 100 | 100 | 100 | 100 | 100 |
| | 4 | hr | 100 | 100 | 100 | 100 | 100 |
| | 1 | Day | 100 | 93 | 100 | 100% | 100 |
| | 2 | Day | 100 | 89 | 99 | 100 | 100 |
| | 3 | Day | 100 | 84 | 99 | 100 | 100 |
| | 4 | Day | 100 | 79 | 98 | 100 | 100 |
| | 7 | Day | 100 | 65 | 96 | 100 | 100 |
| Q1L1-PII-9 | 1 | hr | 100 | 32 | 83 | 95 | 100 |
| | 4 | hr | 100 | 7 | 70 | 95 | 100 |
| | 1 | Day | 100 | 0 | 29 | 92 | 100 |
| | 2 | Day | 100 | | 9 | 85 | 100 |
| | 3 | Day | 100 | | 0 | 83 | 100 |
| | 4 | Day | 100 | | | 78 | 100 |
| | 7 | Day | 100 | | | 70 | 100 |
| Q2L1-PII-1 | 1 | hr | 100 | 100 | 100 | 100 | 100 |
| | 4 | hr | 100 | 100 | 100 | 100 | 100 |
| | 1 | Day | 100 | 100 | 100 | 100 | 100 |
| | 2 | Day | 100 | 100 | 100 | 100 | 100 |
| | 3 | Day | 100 | 96 | 100 | 100 | 100 |
| | 4 | Day | 100 | 95 | 100 | 100 | 100 |
| | 7 | Day | 92 | 91 | 100 | 100 | 100 |
| Q2L6-PII-9 | 1 | hr | | | | | 100 |
| | 4 | hr | 100 | 8 | 71 | 100 | 100 |
| | 1 | Day | 100 | 0 | 25 | 92 | 100 |
| | 2 | Day | 100 | | 8 | 86 | 100 |
| | 3 | Day | 100 | | 2 | 82 | 100 |
| | 4 | Day | 100 | | 0 | 69 | 100 |
| | 7 | Day | 92 | | | 66 | 100 |
| Q2L7-PII-1 | 1 | hr | 97 | 100 | 100 | 100 | 100 |
| | 4 | hr | 97 | 100 | 100 | 100 | 100 |
| | 1 | Day | 97 | 100 | 100 | 100 | 100 |
| | 2 | Day | 97 | 100 | 100 | 100 | 100 |
| | 3 | Day | 97 | 96 | 100 | 100 | 100 |
| | 4 | Day | 97 | 95 | 100 | 100 | 100 |
| | 7 | Day | 97 | 91 | 100 | 100 | 100 |

TABLE 7a
Chemical Stability of Quenched vcPAB-Linkers

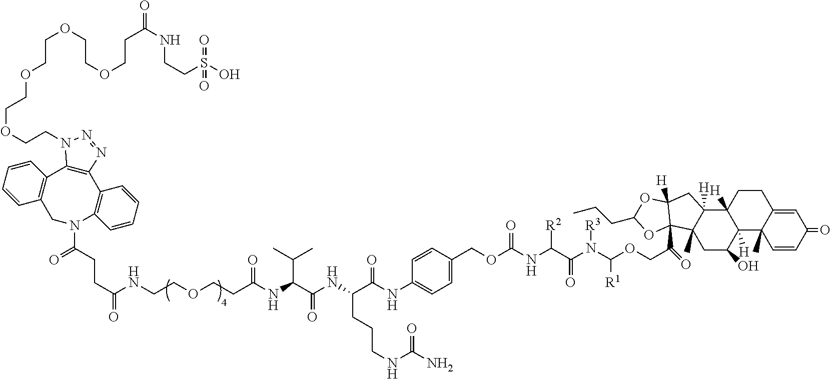

| Structures | | | Linker-payloads | Time | pH 4.0 | | pH 5.0 | | pH 7.4 | |
|---|---|---|---|---|---|---|---|---|---|---|
| R² | R³ | R¹ | Cpd# | (day) | LP* | P** | LP* | P** | LP* | P** |
| H | H | H | Q1L1-PII-1 | 1 | 100 | 0 | 100 | 0 | 100 | 0 |
| | | | | 2 | 100 | 0 | 100 | 0 | 100 | 0 |
| | | | | 7 | 100 | 0 | 100 | 0 | 100 | 0 |
| H | H | Et | Q1L1-PII-6 | 1 | 64 | 15 | 100 | 0 | 100 | 0 |
| | | | | 2 | 46 | 38 | 98 | 2 | 100 | 0 |
| | | | | 7 | 21 | 79 | 97 | 6 | 100 | 0 |
| H | H | Bn | Q1L1-PII-7 | 1 | 100 | 0 | 100 | 0 | 100 | 0 |
| | | | | 2 | 94 | 5 | 100 | 0 | 100 | 0 |
| | | | | 7 | 82 | 17 | 100 | 0 | 100 | 0 |
| H | Me | H | Q1L1-PII-8 | 1 | 100 | 0 | 100 | 0 | 100 | 0 |
| | | | | 2 | 99 | 0 | 100 | 0 | 100 | 0 |
| | | | | 7 | 96 | 3 | 100 | 0 | 100 | 0 |
| Me | Me | H | Q1L1-PII-11 | 1 | 100 | 0 | 100 | 0 | 100 | 0 |
| | | | | 2 | 100 | 0 | 100 | 0 | 100 | 0 |
| | | | | 7 | 100 | 0 | 100 | 0 | 100 | 0 |
| 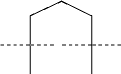 | | H | Q1L1-PII-12 | 1 | 100 | 0 | 100 | 0 | 100 | 0 |
| | | | | 2 | 100 | 0 | 100 | 0 | 100 | 0 |
| | | | | 7 | 100 | 0 | 100 | 0 | 100 | 0 |
| H | 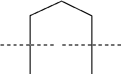 | | Q1L1-PII-9 | 1 | 70 | 26 | 95 | 4 | 100 | 0 |
| | | | | 2 | 29 | 67 | 92 | 7 | 100 | 0 |
| | | | | 7 | 0 | 97 | 70 | 27 | 100 | 0 |

*LP: percentage of linker-payloads remaining after the corresponding time.
**P: percentage of budesonide released from the linker-payloads after the incubation time.

TABLE 7b
Chemical Stability of Quenched Linkers

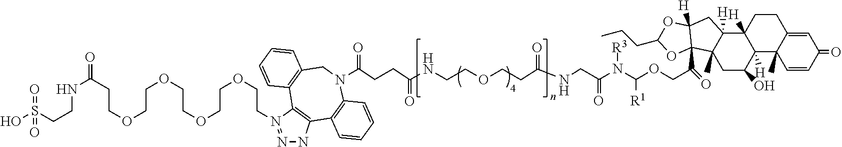

| | | | Linker- | | | | | | | | | |
|---|---|---|---|---|---|---|---|---|---|---|---|---|
| Structures | | | payloads | Time | pH 4.0 | | pH 5.0 | | pH 6.0 | | pH 7.4 | |
| n | R³ | R¹ | Cpd# | (day) | LP* | P** | LP* | P** | LP* | P** | LP* | P** |
| 0 | H | H | Q1L7-PII-11 | 1 | 100 | 0 | 100 | 0 | 100 | 0 | 100 | 0 |
| | | | | 2 | 100 | 0 | 100 | 0 | 100 | 0 | 100 | 0 |
| | | | | 7 | 100 | 0 | 100 | 0 | 100 | 0 | 100 | 0 |
| 1 | 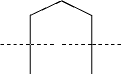 | | Q1L6-PII-9 | 1 | 30 | 69 | 94 | 5 | 100 | 0 | 100 | 0 |
| | | | | 2 | 10 | 89 | 89 | 10 | 100 | 0 | 100 | 0 |
| | | | | 7 | 0 | 99 | 71 | 28 | 92 | 8 | 100 | 0 |
| 1 | 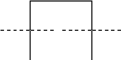 | | Q1L6-PII-13 | 1 | 100 | 0 | 100 | 0 | 100 | 0 | 100 | 0 |
| | | | | 2 | 100 | 0 | 100 | 0 | 100 | 0 | 100 | 0 |
| | | | | 7 | 100 | 0 | 100 | 0 | 100 | 0 | 100 | 0 |

*LP: percentage of linker-payloads remaining after the corresponding time.
**P: percentage of budesonide released from the linker-payloads after the incubation time.

TABLE 7c
Chemical Stability of Linker-budesonide

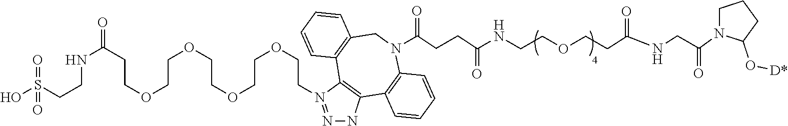

| Structures | Linker-payloads | Time | pH 4.0 | | pH 5.0 | | pH 6.0 | | pH 7.4 | |
|---|---|---|---|---|---|---|---|---|---|---|
| D*—OH | Cpd# | (day) | LP* | P** | LP* | P** | LP* | P** | LP* | P** |
| Budesonide | Q1L6-PII-9 | 1 | 30 | 69.0 | 94.0 | 5.0 | 100 | 0 | 100 | 0 |
| | | 2 | 10 | 89.0 | 89.0 | 10 | 100 | 0 | 100 | 0 |
| | | 7 | 0 | 99.0 | 71.0 | 28 | 92 | 8 | 100 | 0 |

*LP: percentage of linker-payloads remaining after the corresponding time.
**P: percentage of budesonide released from the linker-payloads after the incubation time.

TABLE 7d
Chemical Stability of Glucose Linker-budesonide

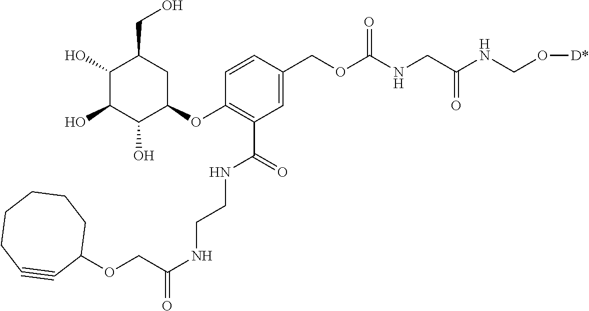

| | Linker- | | | | | | | | | |
|---|---|---|---|---|---|---|---|---|---|---|
| Structures | payloads | Time | pH 4.0 | | pH 5.0 | | pH 6.0 | | pH 7.4 | |
| D*—OH | Cpd# | (day) | LP* | P** | LP* | P** | LP* | P** | LP* | P** |
| Budesonide | L18-PII-1 | 1 | 100 | 0 | 100 | 0 | 100 | 0 | 100 | 0 |
| | | 2 | 100 | 0 | 100 | 0 | 100 | 0 | 100 | 0 |
| | | 7 | 100 | 0 | 100 | 0 | 100 | 0 | 100 | 0 |

*LP: percentage of linker-payloads remaining after the corresponding time.
**P: percentage of budesonide released from the linker-payloads after the incubation time.

### Example 4a

### Metabolic Stability With Human Liver Microsomes

The metabolic stability of payload PIII-1 incubated with human liver microsomes was evaluated and the results were shown in the Table 7e below.

TABLE 7e

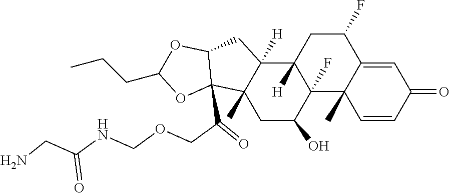

| | | | Percent Remaining (%) | | | | | | |
|---|---|---|---|---|---|---|---|---|---|
| Test | | | 0 | 5 | 15 | 30 | 45 | T_{1/2} | Clᵢₙₜ |
| Article | Species | | min | min | min | min | min | (minute) | (mL/min/kg) |
| ketanserin | human | Mean | 100.00 | 81.45 | 67.51 | 41.90 | 36.47 | 31.50 | 55.18 |
| | | RSD of | 0.09 | 0.09 | 0.01 | 0.05 | 0.07 | | |
| | | Area | | | | | | | |
| | | Ratio | | | | | | | |
| III | human | Mean | 100.00 | 16.15 | 0.75 | 0.76 | 0.14 | 2.14 | 810.85 |
| | | RSD of | 0.05 | 0.08 | 0.16 | 0.17 | 1.41 | | |
| | | Area | | | | | | | |
| | | Ratio | | | | | | | |
| PIII-1 | human | Mean | 100.00 | 59.35 | 23.58 | 6.83 | 2.68 | 7.80 | 222.98 |
| | | RSD of | 0.02 | 0.00 | 0.01 | 0.16 | 0.28 | | |
| | | Area | | | | | | | |
| | | Ratio | | | | | | | |
| | | | Area ratio | | | | | | |
| Test Article | Species | | 0 min | 5 min | 15 min | 30 min | 45 min | | |
| III from P11I-1 | human | Mean | BQL | 0.001 | 0.002 | 0.001 | 0.001 | | |
| | | RSD of Area | N/A | 0.01 | 0.03 | 0.04 | 0.11 | | |
| | | Ratio | | | | | | | |

### Example 5

### Antibodies Conjugated to Steroid Payloads via Hemiaminal Ether Linkers Described Herein are Active in a GR Luciferase Reporter Assay in HEK293/GRLuc/MSR1 Cells

Linker-payloads were generated and conjugated to an anti-macrophage scavenger receptor 1 (MSR1) antibody (H1H21234N-N297Q; HCVR SEQ ID NO:19, LCVR SEQ ID NO:27, N297Q) or a non-binding isotype control antibody (Isotype Control-N297Q) to produce the non-cytotoxic antibody drug conjugates (ncADCs) listed in Table 8. Specifically, a site-specific azido-functionalized N297Q mutated aglycosylated antibody (e.g. targeting MSR1 and non-binding control) conjugate was prepared by incubating the azido-functionalized glutaminyl-modified antibody in an aqueous medium (dissolved 1 mg/mL in PBS, PBS containing 5% glycerol, HBS) with ≥6 molar equivalent of linker-payload (dissolved in a suitable organic solvent, such as DMSO, DMF or DMA, to make the reaction mixture contain 10-20% organic solvent, v/v) at 24° C. to 37° C. for over 6 hours. The progress of the reaction was monitored by ESI-MS. Absence of azido-functionalized antibody indicated completion of the conjugation. The excess linker-payload and organic solvent were removed by SEC (Waters, Superdex 200 HR, 1.0×30 cm, GE Healthcare, flow rate 0.7 mL/min) eluting with pH 7.4 PBS. The purified conjugate was analyzed by UV-Vis, SEC, and ESI-MS. All ESI-MS and DAR values of all antibodies (Ab), azido-functionalized antibody (Ab-N₃), and Antibody GC-Steroid conjugates (Ab-LP) are summarized in Table 8a.

TABLE 8
GR Reporter Activity by ncADCs

| | | | Cell lines | | | |
|---|---|---|---|---|---|---|
| | | | HEK293/GRLuc | | HEK293/GRLuc/MSR1 | |
| Test Article | LP | Payload | EC₅₀ | S/N | EC₅₀ | S/N |
| H1H21234N- | L1-PII-1 | II | >1.00e−07 | 5.4 | 6.09e−010 | 127.7 |
| N297Q-L1-PII-1 | | | | | | |
| H1H21234N- | L1-PIII-1 | III | 5.06e−08 | 16.4 | 9.73e−011 | 143.2 |
| N297Q-L1-PIII-1 | | | | | | |
| H1H21234N- | L2-PIII-1 | PIII-1 | 5.27e−08 | 6.0 | 1.84e−010 | 96.4 |
| N297Q-L2-PIII-1 | | | | | | |
| H1H21234N- | L3-PIII-1 | III | 4.76e−08 | 17.5 | 8.54e−011 | 268.8 |
| N297Q-L3-PIII-1 | | | | | | |
| H1H21234N- | L4-PIII-1 | III | 5.14e−08 | 25.5 | 1.17e−010 | 147.8 |
| N297Q-L4-PIII-1 | | | | | | |
| H1H21234N- | L5-PIII-1 | III | 4.17e−08 | 13.2 | 1.38e−010 | 125.3 |
| N297Q-L5-PIII-1 | | | | | | |
| Isotype Control- | L1-PII-1 | II | >1.00e−07 | 1.1 | >1.00e−07 | 3.8 |
| N297Q-L1-PII-1 | | | | | | |
| Isotype Control- | L1-PIII-1 | III | >1.00e−07 | 17.4 | 5.02e−08 | 148.4 |
| N297Q-L1-PIII-1 | | | | | | |
| Isotype Control- | L2-PIII-1 | PIII-1 | >1.00e−07 | 13.4 | 5.73e−08 | 137.3 |
| N297Q-L2-PIII-1 | | | | | | |
| Isotype Control- | L3-PIII-1 | III | 6.44e−08 | 10.1 | 5.19e−08 | 89.3 |
| N297Q-L3-PIII-1 | | | | | | |
| Isotype Control- | L4-PIII-1 | III | >1.00e−07 | 8.0 | 6.39e−08 | 62.3 |
| N297Q-L4-PIII-1 | | | | | | |
| Isotype Control- | L5-PIII-1 | III | 5.94e−08 | 16.0 | 5.23e−08 | 130.2 |
| N297Q-L5-PIII-1 | | | | | | |
| H1H21234N-N297Q | — | — | >1.00e−07 | 1.1 | >1.00e−07 | 1.0 |

TABLE 8a
List of site-specific Steroid-Antibody
Conjugates ESI-MS and DAR

| LP | | Ab, Ab-N₃, or Ab-LP | | |
|---|---|---|---|---|
| LP# | M.W. (Da) | Name | MW (Da) | DAR |
| | | Anti-MSR1 Ab | 145777 | |
| PEG₃-N₃ | 218.26 | Anti-MSR1 Ab-N₃ | 146587 | 4 |
| L1-PII-1 | 1456.71 | Anti-MSR1 Ab-L1-PII-1 | 152411 | 4 |
| L1-PIII-1 | 1492.69 | Anti-MSR1 Ab-L1-PIII-1 | 152564 | 4 |
| L2-PIII-1 | 1621.81 | Anti-MSR1 Ab-L2-PIII-1 | 153070 | 4 |
| L3-PIII-1 | 1348.52 | Anti-MSR1 Ab L3-PIII-1 | 151989 | 4 |
| L4-PIII-1 | 1101.22 | Anti-MSR1 Ab-L4-PIII-1 | 151000 | 4 |
| L5-PIII-1 | 1155.23 | Anti-MSR1 Ab-L5-PIII-1 | 151216 | 4 |
| | | Isotype Control Ab | 145441 | |
| PEG₃-N₃ | 218.26 | Isotype control | 146235 | 4 |
| | | Ab-PEG₃-N₃ | | |
| L1-PII-1 | 1456.71 | Isotype Control Ab-L1- | 152072 | 4 |
| | | PII-1 | | |
| L1-PIII-1 | 1492.69 | Isotype Control Ab-L1- | 152230 | 4 |
| | | PIII-1 | | |
| L2-PIII-1 | 1621.81 | Isotype Control Ab L2- | 152733 | 4 |
| | | PIII-1 | | |
| L3-PIII-1 | 1348.52 | Isotype Control Ab-L3- | 151646 | 4 |
| | | PIII-1 | | |
| L4-PIII-1 | 1101.22 | Isotype Control Ab L4- | 150658 | 4 |
| | | PIII-1 | | |
| L5-PIII-1 | 1155.23 | Isotype Control Ab L5- | 150880 | 4 |
| | | PIII-1 | | |

Activity of anti-MSR1 ncADCs were tested using an MSR1 ADC steroid responsive luciferase reporter assay. To generate the assay cell line, HEK293 cells were co-transfected with the pBind-GR (Promega, Cat #E158A) and pGL4.35 [Luc2P/9xGAL4UAS/Hygro] (Promega, Cat #E137A) vectors. The pBind-GR vector expresses a fusion protein consisting of the GR ligand binding and the yeast Gal4DNA-binding domain, which binds to the Gal4 upstream activation sequence (UAS) in the luciferase expression vector, that can induce luciferase expression following GR agonist binding. The cells were selected for at least 2 weeks in G418+hygromycin and the resulting cell line is referred to as HEK293/GRLuc. HEK293/GRLuc cells were then transduced with a human MSR1 lentiviral expression vector and 48 hours later cells were sorted for MSR1 positive cells. The resulting cell line is referred to as HEK293/GRLuc/MSR1.

For the assay, cells were seeded into 96 well plates at 20,000 cells/well in assay media (DME high glucose supplemented with 10% FBS, 100 units/mL Penicillin, 100 ug/mL Streptomycin, and 53 ug/mL glutamine) one day prior to assay. Three-fold serial dilutions of ncADCs in assay buffer were added to the cells for 48 hours. The last well in the plate served as blank control containing only the assay media and was plotted as a continuation of the 3-fold serial dilution. Luciferase activity was determined using the One-Glo™ reagent (Promega, Cat #E6130). Relative light units (RLUs) were measured on an Envision luminometer (PerkinElmer) and EC₅₀ values were determined using a four-parameter logistic equation over a 10-point dose response curve (GraphPad Prism). The signal to noise (S/N) was determined by taking the ratio of the highest RLU on the dose response curve to the RLU in the untreated wells.

As shown in Table 8, the anti-MSR1 (H1H21234N-N297Q) ncADCs EC₅₀ values ranged from 85 pM to 609 pM and S/N values ranged from 96.4 to 268.8 in the HEK293/GRLuc/MSR1 cells. All isotype control ncADCs EC₅₀ values were greater than 50 nM and the S/N values ranged from 3.8 to 148.4 in the HEK293/GRLuc/MSR1 cell line. All anti-MSR1 ncADC EC₅₀ values were greater than 41 nM in the HEK293/GRLuc cells with S/N values ranging from 5.4 to 25.5. The unconjugated anti-MSR1 did not have activity in any of the tested cell lines.

### Example 6

### Evaluation of Activity of Compound III and PIII-1 in a GR Luciferase Reporter Assay in HEK293/GRLuc Cells and HEK293/GRLuc/MSR1

To test the activity of GR agonist payloads disclosed herein, a cell-based GR responsive luciferase reporter assay was developed as described above.

For the assay, cells were seeded into 96 well plates at 20,000 cells/well in assay media (DME high glucose supplemented with 10% FBS, 100 units/ml Penicillin, 100 ug/ml Streptomycin, and 53 ug/ml glutamine) one day prior to assay. Three-fold serial dilutions of free payloads were prepared in 100% DMSO, transferred to fresh assay media, and added to the cells for 48 hours. The final DMSO concentration was held constant at 0.2%, and the free payload final concentrations ranged from 100 nM to 0.015 nM. The last well in the plate served as blank control containing only the assay media and 0.2% DMSO (untreated well) and was plotted as a continuation of the 3-fold serial dilution. Luciferase activity was determined using the One-Glo™ reagent (Promega, Cat #E6130). Relative light units (RLUs) were measured on an Envision luminometer (PerkinElmer) and EC₅₀ values were determined using a four-parameter logistic equation over a 10-point dose response curve (GraphPad Prism). The signal to noise (S/N) was determined by taking the ratio of the highest RLU on the dose response curve to the RLU in the untreated wells. As shown in Table 8b, payload EC₅₀ values ranged from 54.5 pM nM to 272 pM and S/N values ranged from 138.1 to 179.3 in the HEK293/GRLuc/MSR1 cells. Payload EC₅₀ values ranged from 19.7 pM nM to 213 pM and S/N values ranged from 24.6 to 34.4 in the HEK293/GRLuc cell line.

TABLE 8b
GR Reporter Activity by GR Agonist

| Cell lines | |
|---|---|
| HEK293/GRLuc | HEK293/GRLuc/MSR1 |

| Cpd. No. | EC₅₀ | S/N | EC₅₀ | S/N |
|---|---|---|---|---|
| II | 2.13e−010 | 24.6 | 2.72e−010 | 138.1 |
| III | 1.97e−011 | 34.4 | 2.42e−011 | 156.7 |
| PIII-1 | 4.65e−011 | 32.1 | 5.45e−011 | 179.3 |

### Example 7

### Evaluation of Payload Release

To confirm the release of payloads linked to antibodies via hemiaminal ether linkers, a lysosomal cleavage assay was performed. For the assay, the ncADCs were added to freshly prepared lysosome working solution containing 1× catabolic buffer (Xenotech Cat #K5200) and 0.125 mg/mL human liver lysosome proteins (Xenotech Cat #H0610.L). 200 μL of resulting mixture with 1 μM payload-equivalent ncADC was incubated at 37° C. with gentle shaking over a 24-hour period. A 20 μL aliquot was taken at 0, 0.5, 1.0, 2.0, 4.0, 8.0 and 24 hours and immediately transferred to a plate containing 80 μL cold acetonitrile to deactivate the lysosomal enzymes and precipitate the proteins. After centrifugation at 2,000 rpm for 5 minutes, aliquots of supernatant were diluted with an equal volume of water for LC-MS analysis to determine the amount of precursor and payload released from ncADC.

As shown in Table 9, the ncADC steroid conjugate with the linker vcPAB-gly-NHCH₂ (H1H21234N-N297Q-L1-PIII-1) released 164 nM steroid III at the 24-hour time point. The ncADC steroid conjugate with the linker GGFG-NHCH₂ (H1H21234N-N297Q-L3-PIII-1) released 345 nM steroid III at the 24-hour time point.

Without being bound by any particular theory, under certain conditions, payloads having a hemiaminal ether linker residue (“payload precursor” or “prodrug”) are initially released, followed by release of the ultimate payload. As shown in Table 10, the ncADC steroid conjugate with the linker vcPAB-gly-NHCH₂ (H1H21234N-N297Q-L1-PIII-1) released 725 nM of biologically active steroid precursor PIII-1 at the 24-hour time point. The ncADC steroid conjugate with the linker GGFG-NHCH2 (H1H21234N-N297Q-L3-PIII-1) released 4.3 nM of biologically active steroid precursor PIII-1 at the 24-hour time point.

TABLE 9
Payload Release

| | | Payload | Linker- | Released Payload Concentration (nM) | | | | | | |
|---|---|---|---|---|---|---|---|---|---|---|
| ncADC | Payload | Type | Type | 0 | 0.5 | 1.0 | 2.0 | 4.0 | 8.0 | 24 |
| H1H21234N- | III | Steroid | vcPAB- | 0 | 0.0 | 0.0 | 3.6 | 24.1 | 80.0 | 164.1 |
| N297QL1- | | | gly- | | | | | | | |
| PIII-1 | | | NHCH₂ | | | | | | | |
| H1H21234N- | III | Steroid | GGFG- | 10.1 | 30.5 | 32.9 | 44.9 | 101 | 175 | 345 |
| N297Q-L3- | | | NHCH₂ | | | | | | | |
| PIII-1 | | | | | | | | | | |

TABLE 10
Payload Precursor Release

| | | Payload | Linker- | Released Payload Precursor Concentration (nM) | | | | | | |
|---|---|---|---|---|---|---|---|---|---|---|
| ncADC | Payload | Type | Type | 0 | 0.5 | 1.0 | 2.0 | 4.0 | 8.0 | 24 |
| H1H21234N- | III | Steroid | vcPAB- | 0 | 126 | 256 | 296 | 374 | 615 | 725 |
| N297Q L1- | | | gly- | | | | | | | |
| PIII-1 | | | NHCH₂ | | | | | | | |
| H1H21234N- | III | Steroid | GGFG-NHCH₂ | 0 | 0 | 0 | 0 | 0 | 1.5 | 4.3 |
| N297Q-L3- | | | | | | | | | | |
| PIII-1 | | | | | | | | | | |

### Example 8

Antibody-drug conjugates having an anti-macrophage scavenger receptor 1 (MSR1) antibody (H1H21234N-N297Q) or a non-binding isotype control antibody (Isotype Control) were conjugated to the linker-payloads listed in Table 11 and evaluated following the procedure described in Example 5. The H1H21234N-N297Q provides a four DAR ADC. As shown in Table 11, the anti-MSR1 (H1H21234N-N297Q) ADCs EC₅₀ values ranged from 636 pM to 3.96 nM and signal to noise (S/N) values ranged from 120.1 to 280.3 in the HEK293/GRLuc/MSR1 cells. All isotype control EC₅₀ values were >35 nM and the S/N values ranged from 1.9 to 153.5 in the HEK293/GRLuc/MSR1 cell line. All ADC EC₅₀ values were >100 nM in the HEK293/GRLuc cells with S/N values ranging from 1.0 to 16.8. The unconjugated anti-MSR1 antibody did not have activity in any of the tested cell lines. The free payload, budesonide, had activity in both cell lines with EC₅₀ values of 710 pM and 1.19 nM and S/N of 23.3 and 135.4 in the HEK293/GRLuc and HEK293/GRLuc/MSR1 cell lines, respectively.

TABLE 11
GR Reporter Activity by ADCs

| | | Cell lines | | | |
|---|---|---|---|---|---|
| | | HEK293/GRLuc | | HEK293/GRLuc/MSR1 | |
| Test Article | Payload | EC₅₀ | S/N | EC₅₀ | S/N |
| H1H21234N-N297Q-L1-PII-1 | II | >1.0E−07 | 4.5 | 8.40E−10 | 222.1 |
| H1H21234N-N297Q-L14-PII-1 | II | >1.0E−07 | 16.8 | 6.36E−10 | 120.1 |
| H1H21234N-N297Q-L6-PII-9 | II | >1.0E−07 | 15.8 | 3.96E−09 | 149.9 |
| H1H21234N-N297Q-L13-PII-1 | II | >1.0E−07 | 3.1 | 2.33E−09 | 225.6 |
| H1H21234N-N297Q-L8-PII-1 | II | >1.0E−07 | 1.4 | 8.99E−10 | 280.3 |
| Isotype Control-L1-PII-1 | II | >1.0E−07 | 1.4 | >1.0E−07 | 2.1 |
| Isotype Control-L14-PII-l | II | >1.0E−07 | 2.3 | 3.52E−08 | 153.5 |
| Isotype Control-L6-PII-9 | II | >1.0E−07 | 12.3 | >1.0E−07 | 47.7 |
| Isotype Control-L13-PII-1 | II | >1.0E−07 | 1.0 | >1.0E−07 | 1.9 |
| Isotype Control-L8-PII-1 | II | >1.0E−07 | 1.1 | >1.0E−07 | 2.0 |
| II | II | 7.10E−10 | 23.3 | 1.19E−09 | 135.4 |
| H1H21234N-N297Q | NA | >1.0E−07 | 1.3 | >1.0E−07 | 1.4 |

1. A compound having the following structure 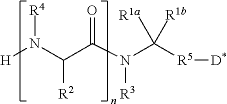
or a pharmaceutically acceptable salt thereof, wherein
R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 3-, 4-, 5-, 6-, 7-, or 8-membered heterocyclyl;
R² is hydrogen, alkylene, heteroalkylene, or an amino acid side chain, wherein when R² is alkylene or heteroalkylene, the alkylene or heteroalkylene is further bonded to R³ to form a 4-, 5, or 6-membered heterocyclyl;
R³ is hydrogen, alkyl, alkylene, or heteroalkylene, wherein when R³ is alkylene or heteroalkylene, the alkylene or heteroalkylene is further bonded to R^{1a} or R² to form the 3-, 4-, 5-, 6-, 7-, or 8-membered heterocyclyl;
R⁴ is hydrogen or alkyl;
R⁵ is oxygen, NR⁶, or sulfur;
R⁶ is hydrogen or alkyl;
D* is acyl, or a residue of a biologically active compound comprising hydroxyl, amino, or thiol; and
n is zero, one, two, three, four, five, or six.

2. The compound of claim 1, having the following structure 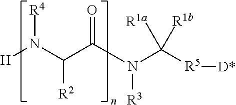
or a pharmaceutically acceptable salt thereof, wherein
R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl;
R² is hydrogen, alkylene, heteroalkylene, or an amino acid side chain, wherein when R² is alkylene or heteroalkylene, the alkylene or heteroalkylene is further bonded to R³ to form a 4-, 5, or 6-membered heterocyclyl;
R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl;
R⁴ is hydrogen or alkyl;
R⁵ is oxygen, NR⁶, or sulfur;
R⁶ is hydrogen or alkyl;
D* is acyl, or a residue of a biologically active compound comprising hydroxyl, amino, or thiol; and
n is zero, one, two, three, four, or five.

3. The compound of claim 2 having the following structure 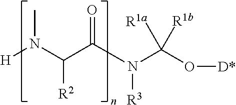
or a pharmaceutically acceptable salt thereof, wherein
R^{1a} and R^{1b} are, independently, hydrogen, alkyl, alkoxy, alkenyl, alkynyl, aryl, arylalkyl, heteroaryl, or alkylene, wherein when R^{1a} is alkylene, the alkylene is further bonded to R³ to form a 4-, 5-, or 6-membered heterocyclyl;
R² is hydrogen or an amino acid side chain;
R³ is hydrogen, alkyl, or alkylene, wherein when R³ is alkylene, the alkylene is further bonded to R^{1a} to form the 4-, 5-, or 6-membered heterocyclyl;
D* is acyl, or a residue of a biologically active compound comprising hydroxyl; and
n is zero, one, two, three, four, or five.

4. The compound of claim 2, wherein the compound has the following structure

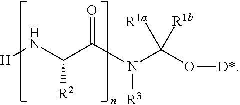

5. The compound of any preceding claim, wherein R^{1a} and R^{1b} are each hydrogen.

6. The compound of any preceding claim, wherein n is two.

7. The compound of any preceding claim, wherein n is two and R² is hydrogen or methyl.

8. The compound of any preceding claim, wherein n is two, R² is hydrogen or methyl, R³ is hydrogen, and D* is a residue of a biologically active compound comprising hydroxyl.

9. The compound of claim 8, wherein the compound is selected from the group consisting of: 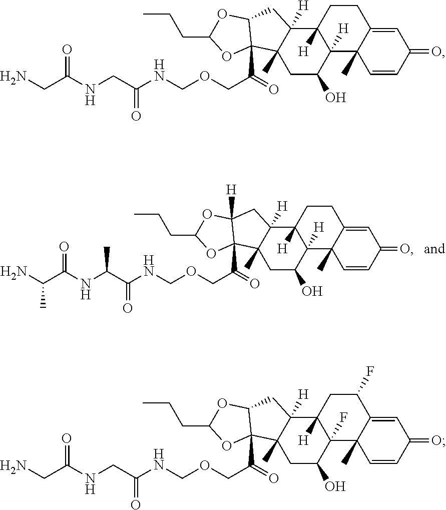
or
a pharmaceutically acceptable salt thereof.

10. The compound of claim 5, wherein n is one.

11. The compound of claim 10, wherein R² is hydrogen, methyl, or —CH₂Ph.

12. The compound of claim 11, wherein R³ is hydrogen.

13. The compound of claim 12, wherein D* is a residue of a biologically active compound comprising hydroxyl.

14. The compound of claim 13, wherein the compound is selected from the group consisting of: 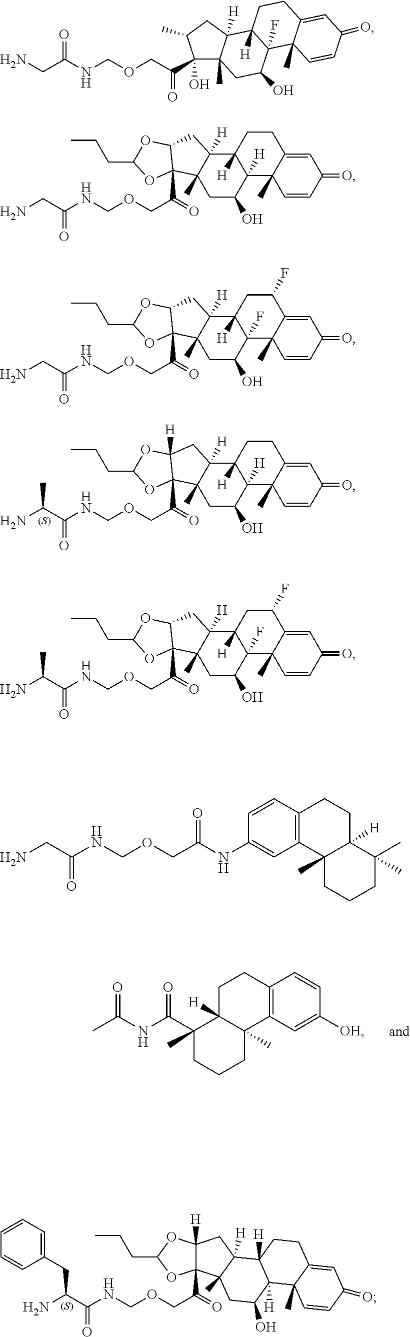
or
a pharmaceutically acceptable salt thereof.

15. The compound of claim 10, wherein R² is hydrogen or methyl.

16. The compound of claim 15, wherein R³ is alkyl.

17. The compound of claim 16, wherein D* is a residue of a biologically active compound comprising hydroxyl.

18. The compound of claim 17, wherein the compound is selected from the group consisting of 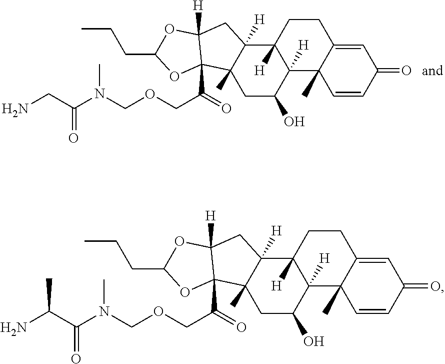
or a pharmaceutically acceptable salt thereof.

19. The compound of claim 2, wherein R^{1a} is alkyl or arylalkyl, and R^{1b} is hydrogen.

20. The compound of claim 19, wherein n is one.

21. The compound of claim 20, wherein R² is hydrogen.

22. The compound of claim 21, wherein R³ is hydrogen.

23. The compound of claim 22, wherein D* is a residue of a biologically active compound comprising hydroxyl.

24. The compound of claim 23, wherein the compound is selected from the group consisting of 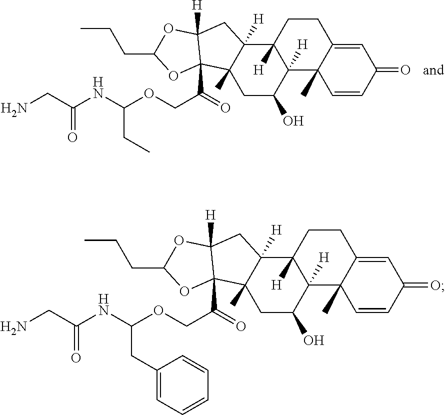
or a pharmaceutically acceptable salt thereof.

25. The compound of claim 2, wherein R^{1a} is alkylene, where the alkylene is further bonded to R³ to form a 4-, 5-, or 6-membered heterocyclyl;
R^{1b} is hydrogen; and
R³ is alkylene further bonded to R^{1a} to form the 4-, 5-, or 6-membered heterocyclyl.

26. The compound of claim 25, wherein n is one.

27. The compound of claim 26, wherein R² is hydrogen.

28. The compound of claim 27, wherein D* is a residue of a biologically active compound comprising hydroxyl.

29. The compound of claim 28, wherein the compound is selected from the group consisting of 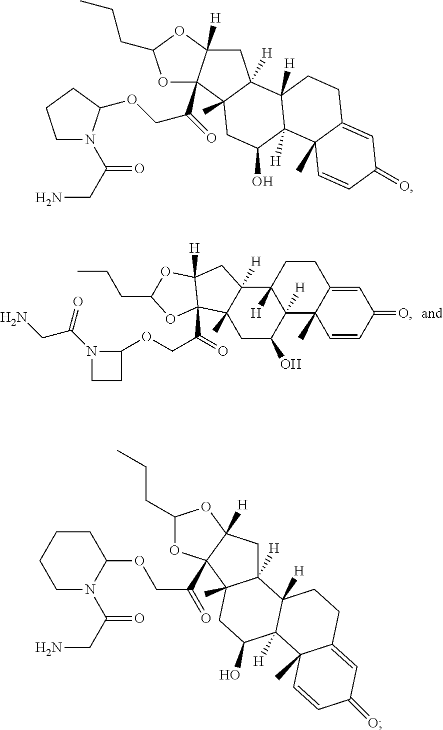
or a pharmaceutically acceptable salt thereof.

30. The compound of claim 10, wherein the compound is

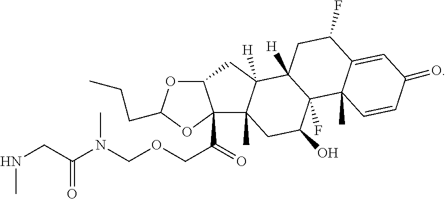 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCN(C)C(=O)CNC)O1]

31. The compound of claim 5, wherein the compound is selected from the group consisting of

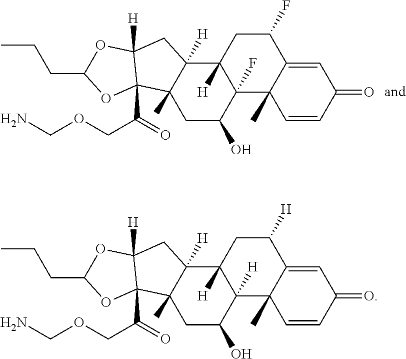

32. The compound of claim 10, wherein R² is alkylene, wherein the alkylene is further bonded to R³ to form a 6-membered heterocyclyl; and
R³ is alkylene, wherein the alkylene is further bonded to R² to form the 6-membered heterocyclyl.

33. The compound of claim 32, wherein D* is a residue of a biologically active compound comprising hydroxyl.

34. The compounds of claim 33, wherein the compound is 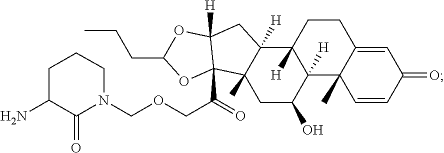
or a pharmaceutically acceptable salt thereof.

35. A compound having the following structure 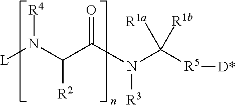
or a pharmaceutically acceptable salt thereof, wherein
L is a linker comprising a moiety reactive with an antibody or an antigen binding fragment thereof;
R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or hetereoalkylene is further bonded to R³ to form a 3-, 4-, 5-, 6-, 7-, or 8-membered heterocyclyl;
R² is hydrogen, alkylene, heteroalkylene, or an amino acid side chain, wherein when R² is alkylene or heteroalkylene, the alkylene or heteroalkylene is further bonded to R³ to form a 4-, 5, or 6-membered heterocyclyl;
R³ is hydrogen, alkyl, alkylene, or heteroalkylene, wherein when R³ is alkylene or heteroalkylene, the alkylene or heteroalkylene is further bonded to R^{1a} or R² to form the 3-, 4-, 5-, 6-, 7-, or 8-membered heterocyclyl;
R⁴ is hydrogen or alkyl;
R⁵ is oxygen, NR⁶, or sulfur;
R⁶ is hydrogen or alkyl;
D* is acyl, or a residue of a biologically active compound comprising hydroxyl, amino, or thiol; and
n is zero, one, two, three, four, five, or six.

36. The compound of claim 35, wherein when D* is a residue of a biologically active compound comprising hydroxyl, amino, or thiol, then the biologically active compound or residue thereof is an anti-inflammatory biologically active compound or residue thereof.

37. The compound of claim 36, wherein the anti-inflammatory biologically active compound is a steroid or a residue thereof.

38. The compound of claim 36, wherein the anti-inflammatory biologically active compound is an LXR agonist or a residue thereof.

39. The compound of claim 35, having the following structure 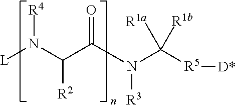
or a pharmaceutically acceptable salt thereof, wherein
L is a linker comprising a moiety reactive with an antibody or an antigen binding fragment thereof;
R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or hetereoalkylene is further bonded to R³ to form a 4-, 5-, or 6-membered heterocyclyl;
R² is hydrogen, alkylene, heteroalkylene, or an amino acid side chain, wherein when R² is alkylene or heteroalkylene, the alkylene or heteroalkylene is further bonded to R³ to form a 4-, 5, or 6-membered heterocyclyl;
R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl;
R⁴ is hydrogen or alkyl;
R⁵ is oxygen, NR⁶, or sulfur;
R⁶ is hydrogen or alkyl;
D* is acyl, or a residue of a biologically active compound comprising hydroxyl, amino, or thiol; and
n is zero, one, two, three, four, five, or six.

40. The compound of claim 39 having the following structure 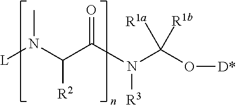
or a pharmaceutically acceptable salt thereof, wherein
L is a linker comprising a moiety reactive with an antibody or an antigen binding fragment thereof;
R^{1a} and R^{1b} are, independently, hydrogen, alkyl, alkoxy, alkenyl, alkynyl, aryl, arylalkyl, heteroaryl, or alkylene, wherein when R^{1a} is alkylene, the alkylene is further bonded to R³ to form a 4-, 5-, or 6-membered heterocyclyl;
R² is hydrogen or an amino acid side chain;
R³ is hydrogen, alkyl, or alkylene, wherein when R³ is alkylene, the alkylene is further bonded to R^{1a} to form the 4-, 5-, or 6-membered heterocyclyl;
D* is acyl, or a residue of a biologically active compound comprising hydroxyl; and
n is zero, one, two, three, four, five, or six.

41. The compound of claim 39 having the following structure 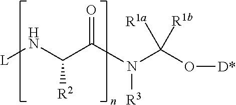
or a pharmaceutically acceptable salt thereof, wherein
L is a linker comprising a moiety reactive with an antibody or an antigen binding fragment thereof;
R^{1a} and R^{1b} are, independently, hydrogen, alkyl, alkoxy, alkenyl, alkynyl, aryl, arylalkyl, heteroaryl, or alkylene, wherein when R^{1a} is alkylene, the alkylene is further bonded to R³ to form a 4-, 5-, or 6-membered heterocyclyl;
R² is hydrogen or an amino acid side chain;
R³ is hydrogen, alkyl, or alkylene, wherein when R³ is alkylene, the alkylene is further bonded to R^{1a} to form the 4-, 5-, or 6-membered heterocyclyl;
D* is acyl, or a residue of a biologically active compound comprising hydroxyl; and
n is zero, one, two, three, four, five, or six.

42. The compound of any one of claims 35-41, wherein the linker further comprises

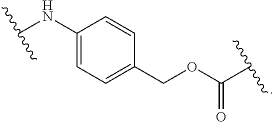 [SMILES: *Nc1ccc(COC(*)=O)cc1]

43. The compound of claim 39, wherein the compound is 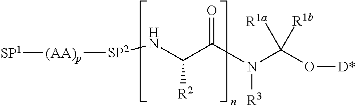
or a pharmaceutically acceptable salt thereof, wherein
SP¹ and SP², when present, are spacer groups wherein SP¹ further comprises a moiety reactive with an antibody or an antigen binding fragment thereof;
each AA is an amino acid; and
p is an integer from 1 to 10.

44. The compound of claim 43, wherein SP¹ comprises a reactive group that comprises an alkyne.

45. The compound of claim 44, wherein the alkyne is capable of participating in a 1,3-cycloaddition reaction.

46. The compound of claim 35, wherein the alkyne is capable of participating in a 1,3-cycloaddition reaction with an azide to form regioisomeric 1,2,3-triazolyl moieties, wherein the azide comprises an azido-functionalized binding agent.

47. The compound of claim 43, wherein SP² comprises

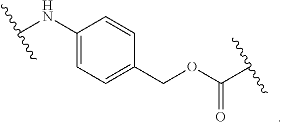 [SMILES: *Nc1ccc(COC(*)=O)cc1]

48. The compound of claim 43, wherein SP² comprises 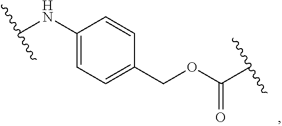 [SMILES: *Nc1ccc(COC(*)=O)cc1]
and R² is hydrogen or alkyl.

49. The compound of claim 46, wherein the alkyne is capable of participating in a 1,3-cycloaddition reaction with an azide to form regioisomeric 1,2,3-triazolyl moieties, the azide comprises an azido-functionalized binding agent, and SP² comprises

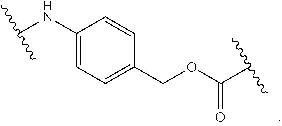 [SMILES: *Nc1ccc(COC(*)=O)cc1]

50. The compound of claim 46, wherein the alkyne is capable of participating in a 1,3-cycloaddition reaction with an azide to form regioisomeric 1,2,3-triazolyl moieties, the azide comprises an azido-functionalized binding agent, SP² comprises 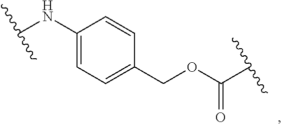 [SMILES: *Nc1ccc(COC(*)=O)cc1]
and R² is hydrogen or alkyl.

51. The compound of claim 43, wherein
R^{1a} and R^{1b} are hydrogen;
R² is hydrogen or methyl;
R³ is hydrogen; and
n is two.

52. The compound of claim 51, wherein D* is a residue of a biologically active compound comprising hydroxyl.

53. The compound of claim 52, selected from the group consisting of 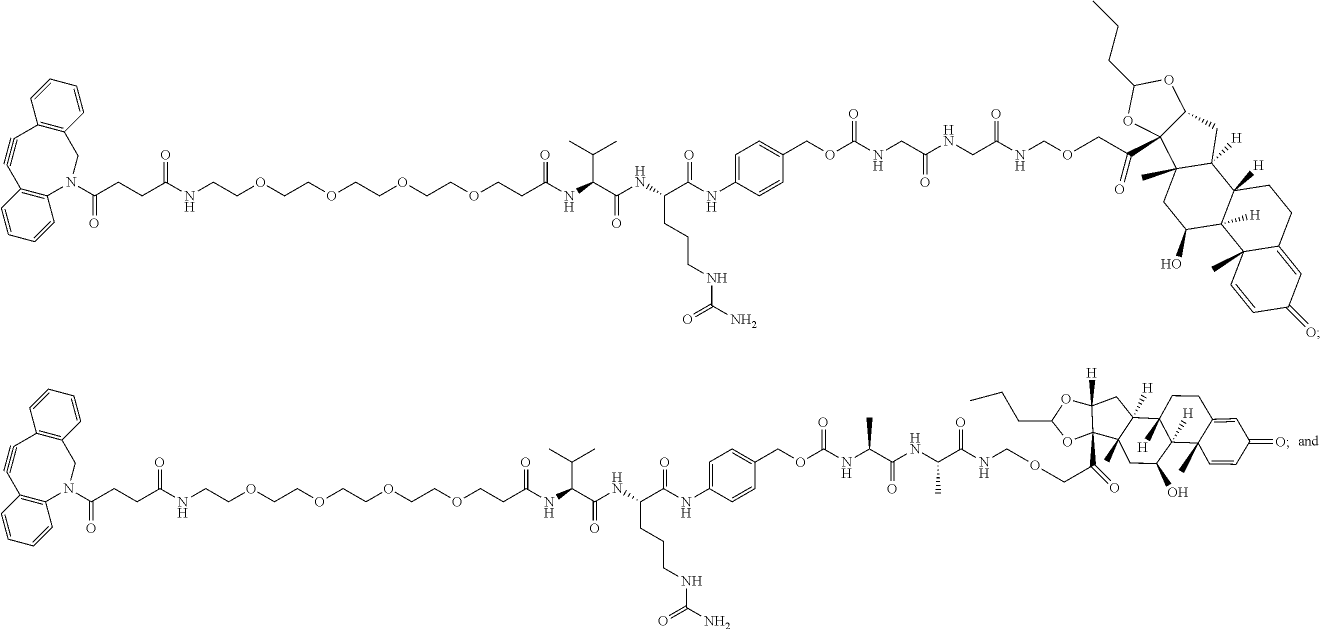 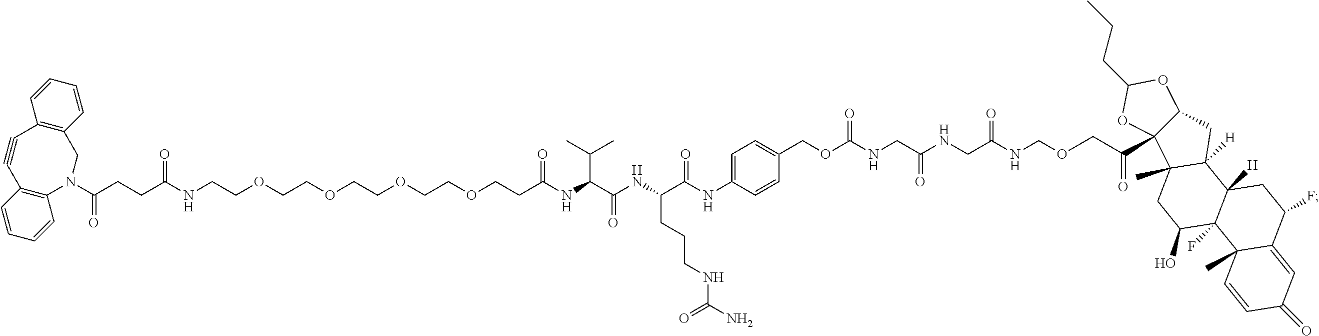 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)CNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)O1]
or a pharmaceutically acceptable salt thereof.

54. The compound of claim 43, wherein
R^{1a} and R^{1b} are hydrogen;
R² is hydrogen, methyl, or —CH₂Ph;
R³ is hydrogen or alkyl; and
n is one.

55. The compound of claim 54, wherein D* is a residue of a biologically active compound comprising hydroxyl.

56. The compound of claim 55, selected from the group consisting of 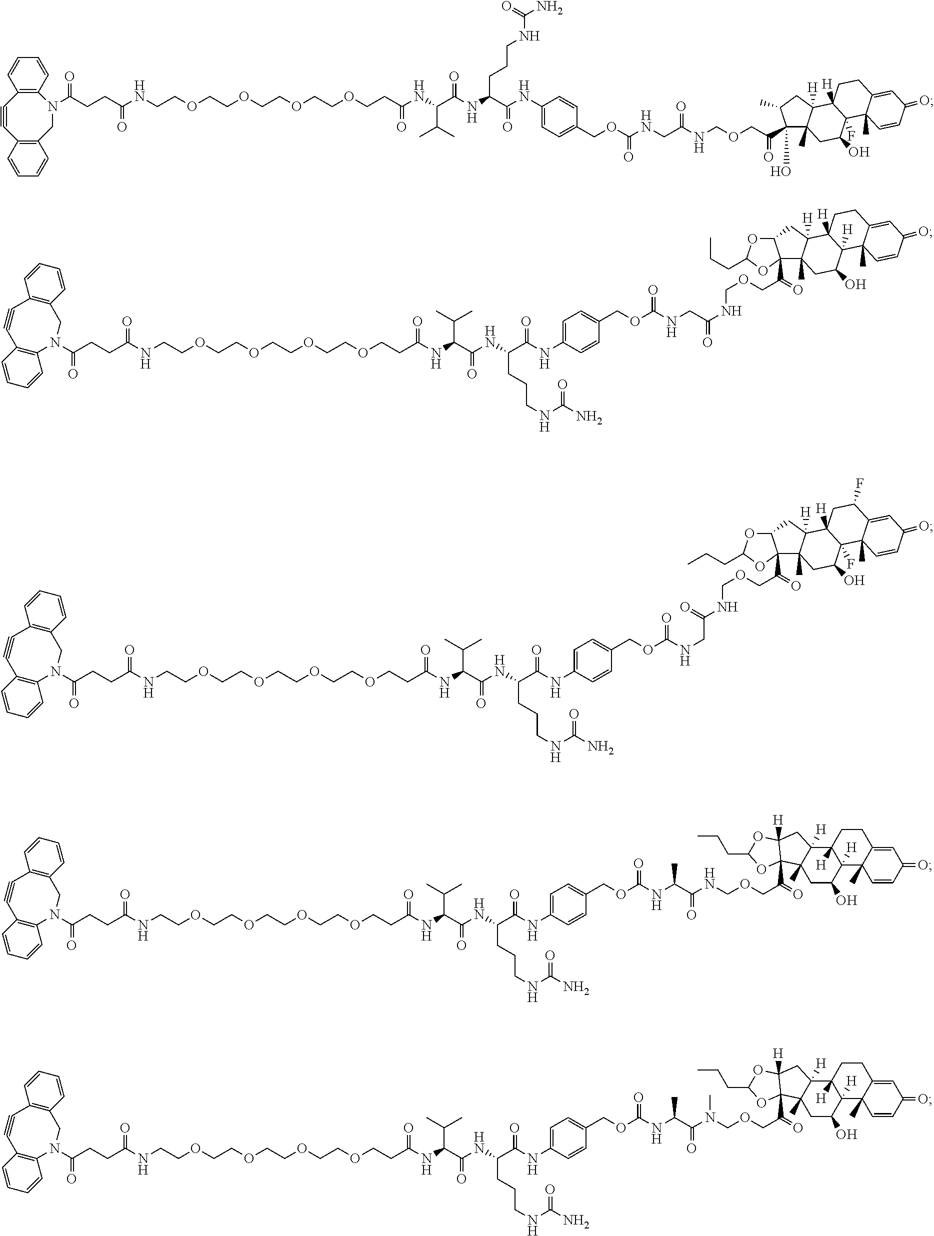 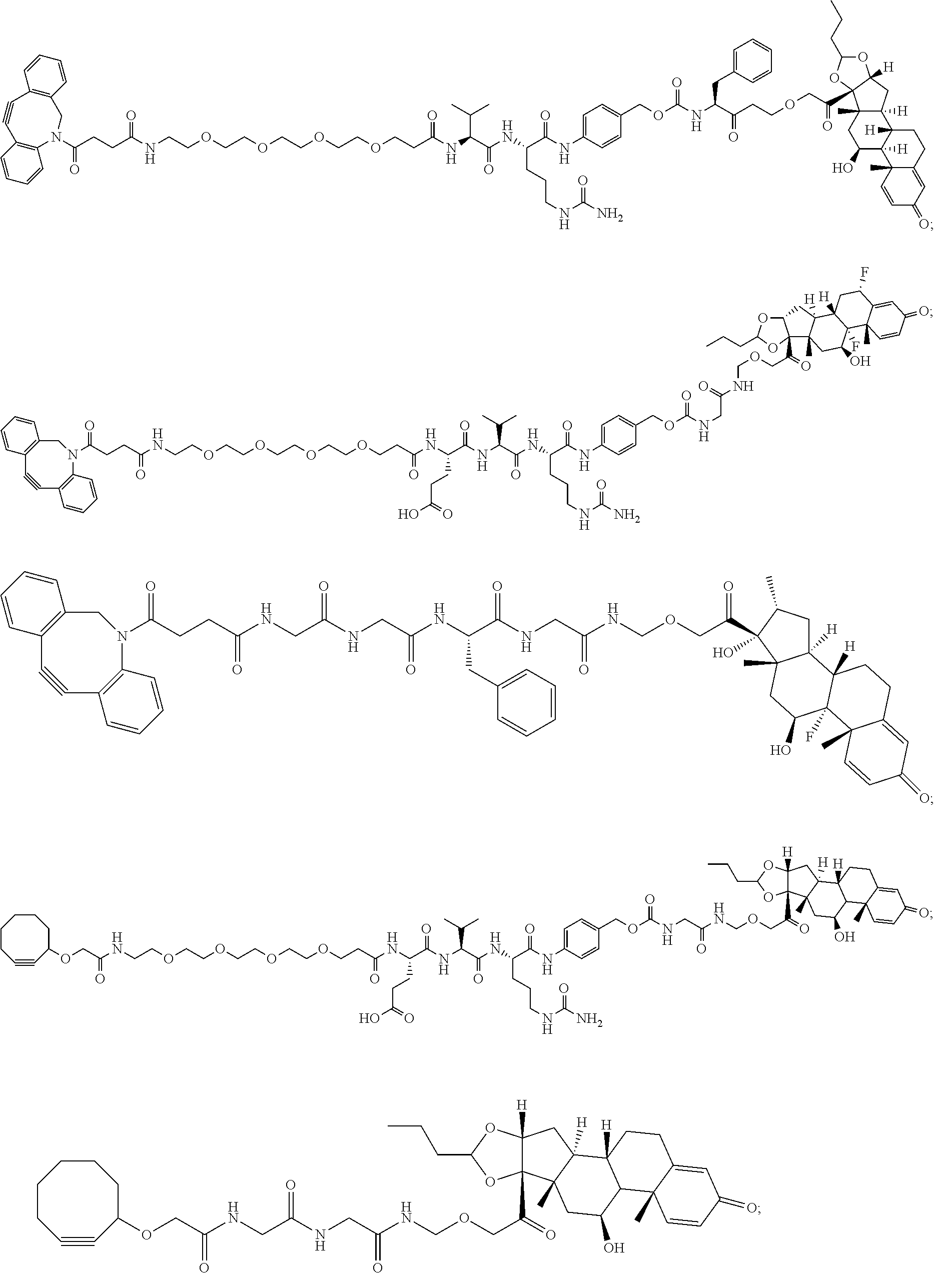 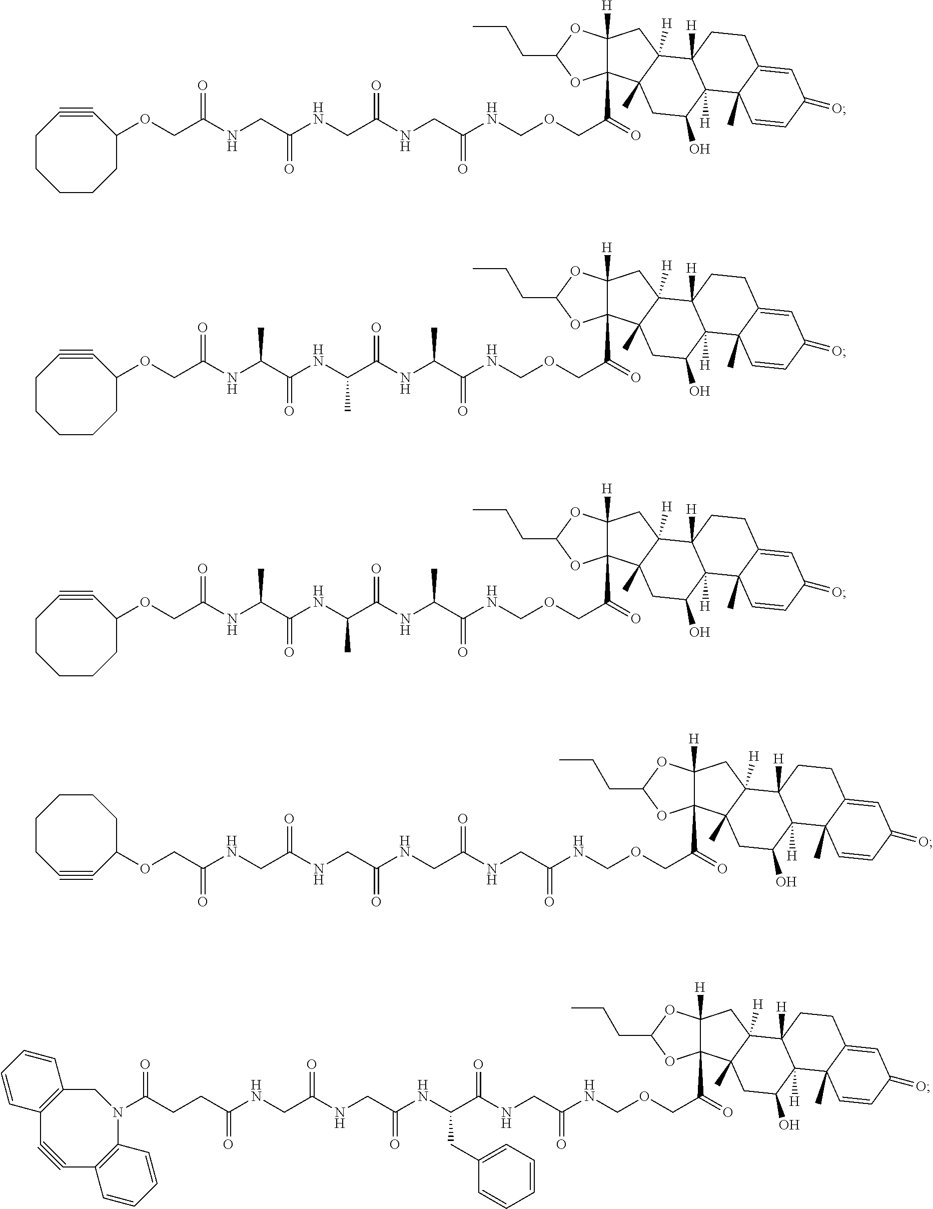 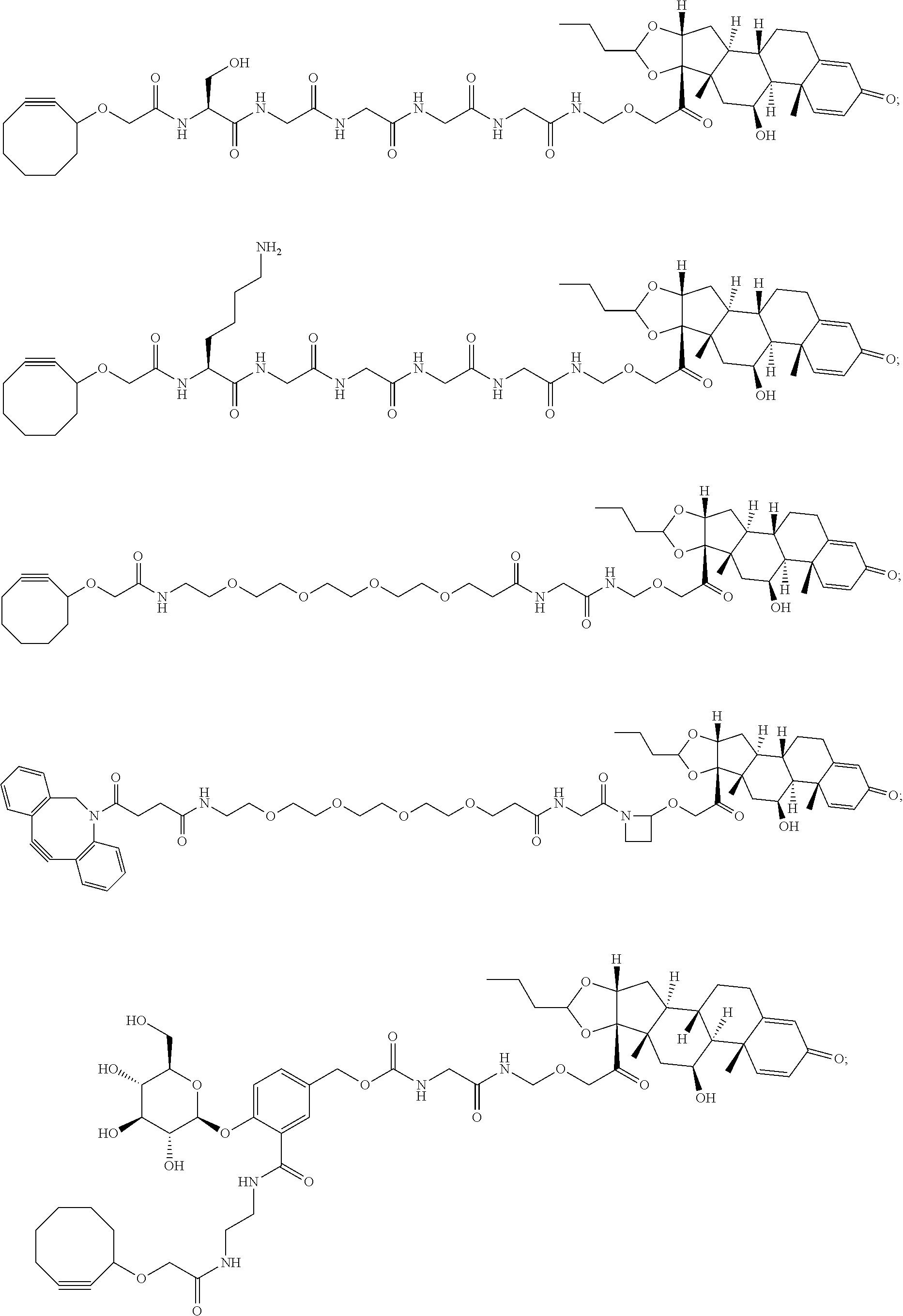 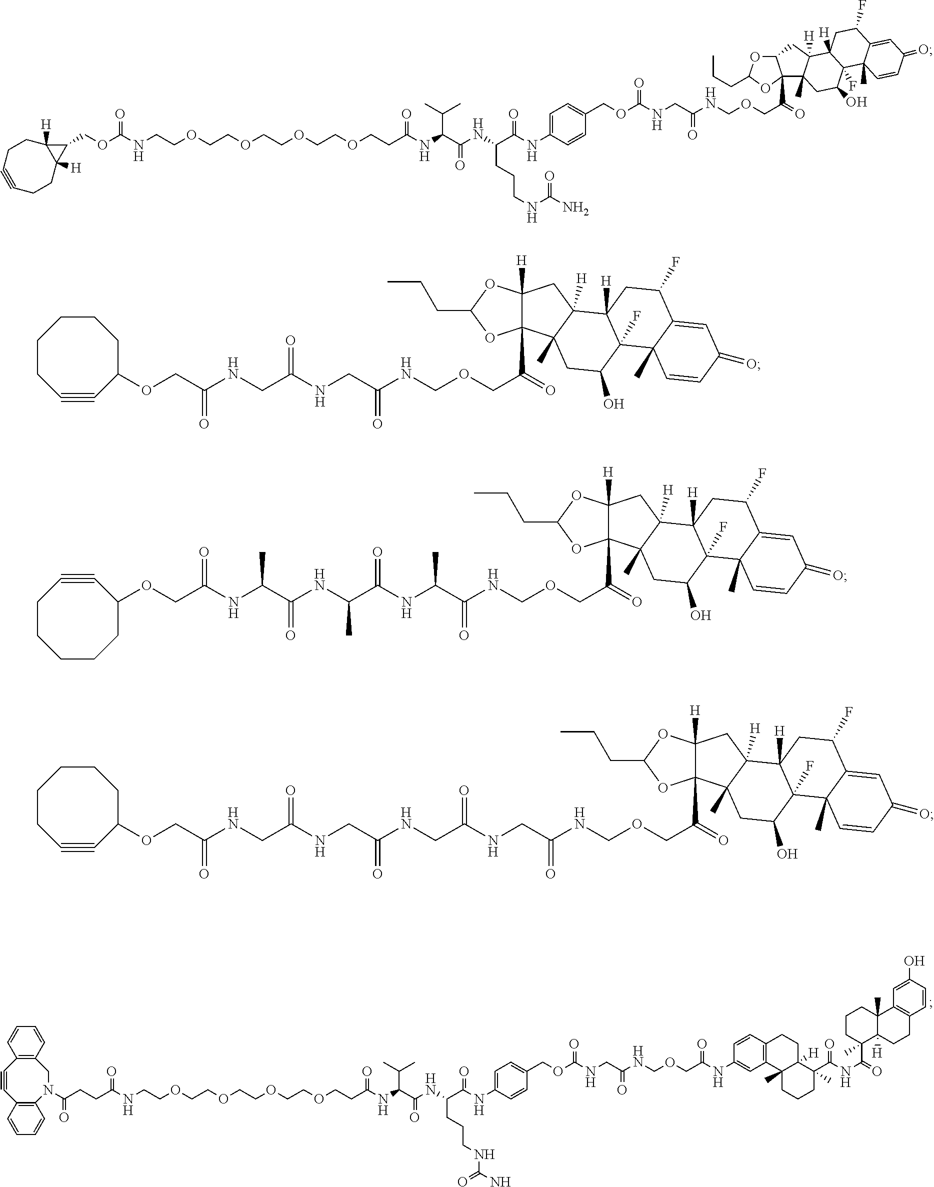 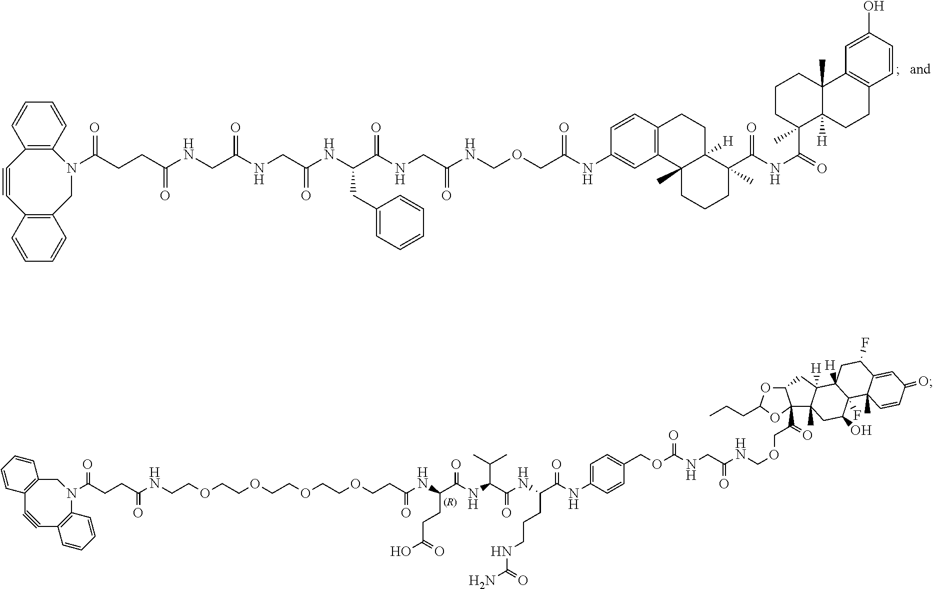
or a pharmaceutically acceptable salt thereof.

57. The compound of claim 43, wherein
R^{1a} and R^{1b} are hydrogen;
R² is hydrogen;
R³ is alkyl; and
n is one.

58. The compound of claim 57, wherein D* is a residue of a biologically active compound comprising hydroxyl.

59. The compound of claim 58 having a structure 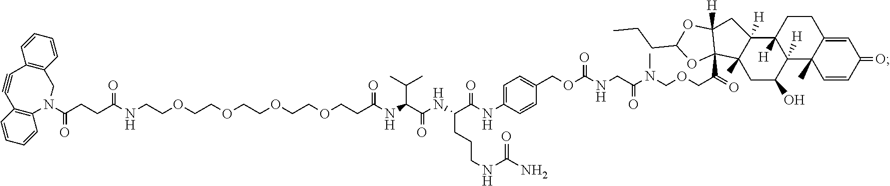
or a pharmaceutically acceptable salt thereof.

60. The compound of claim 43, wherein
R^{1a} is alkyl or arylalkyl;
R^{1b} is hydrogen;
R² is hydrogen;
R³ is hydrogen; and
n is one.

61. The compound of claim 60, wherein D* is a residue of a biologically active compound comprising hydroxyl.

62. The compound of claim 61, selected from the group consisting of 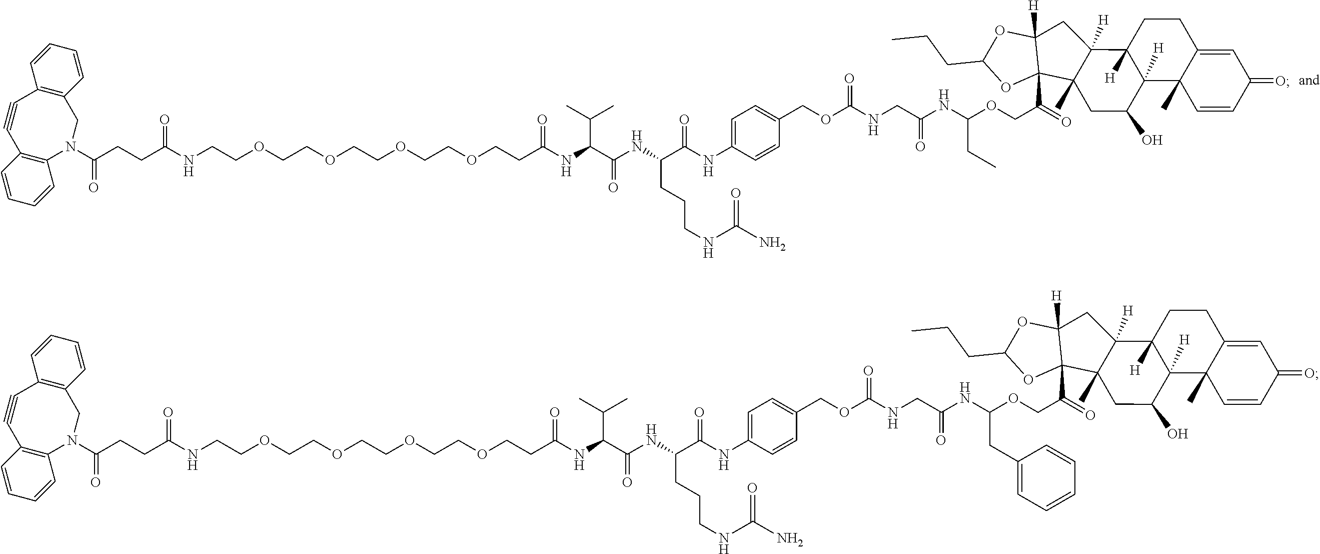
or
a pharmaceutically acceptable salt thereof.

63. The compound of claim 43, wherein
R^{1a} is alkylene, further bonded to R³ to form a 4-, 5-, or 6-membered heterocyclyl;
R^{1b} is hydrogen;
R² is hydrogen;
R³ is alkylene further bonded to R^{1a} to form the 4-, 5-, or 6-membered heterocyclyl; and
n is one.

64. The compound of claim 63, wherein D* is a residue of a biologically active compound comprising hydroxyl.

65. The compound of claim 64, selected from the group consisting of 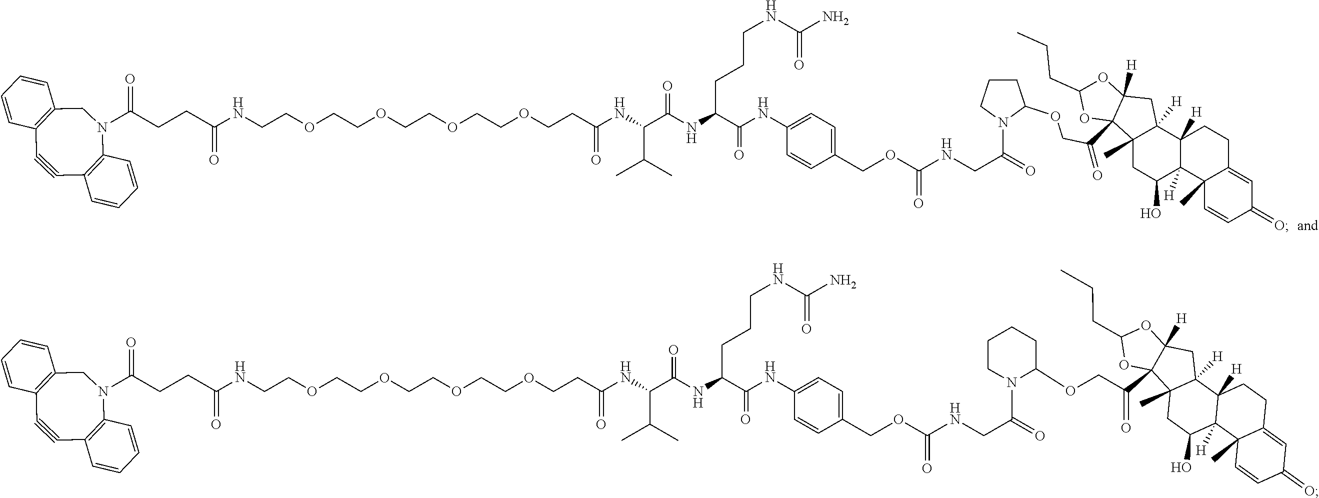 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)O1 ; CCCC1O[C@@H]2C[C@H]3[C@@H]4CCC5=CC(=O)C=C[C@]5(C)[C@H]4[C@@H](O)C[C@]3(C)[C@]2(C(=O)COC2CCCCN2C(=O)CNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)O1]
or
a pharmaceutically acceptable salt thereof.

66. The compound of claim 64, having the structure 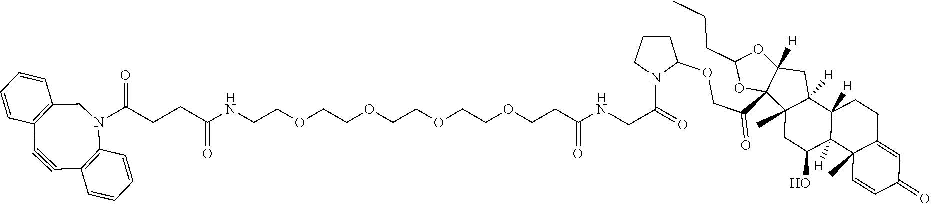 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CNC(=O)CCOCCOCCOCCOCCNC(=O)CCC(=O)N2Cc3ccccc3C#Cc3ccccc32)O1]
or a pharmaceutically acceptable salt thereof.

67. The compound of claim 39, wherein
R^{1a} and R^{1b} are hydrogen;
R² is hydrogen;
R³ is hydrogen; and
n is one.

68. The compound of claim 67, wherein D* is a residue of a biologically active compound comprising hydroxyl.

69. The compound of claim 68, having the following structure 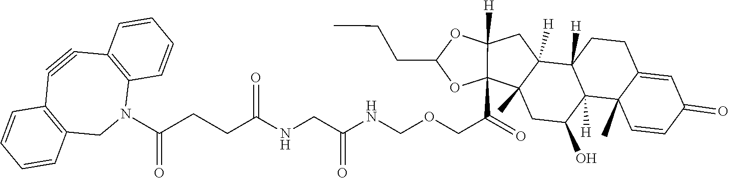 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COCNC(=O)CNC(=O)CCC(=O)N2Cc3ccccc3C#Cc3ccccc32)O1]
or
a pharmaceutically acceptable salt thereof.

70. The compound of claim 39, wherein
R^{1a} and R^{1b} are hydrogen;
R² is hydrogen or —CH₂Ph;
R³ is hydrogen; and
n is four.

71. The compound of claim 70, wherein D* is a residue of a biologically active compound comprising hydroxyl.

72. The compound of claim 71, selected from the group consisting of 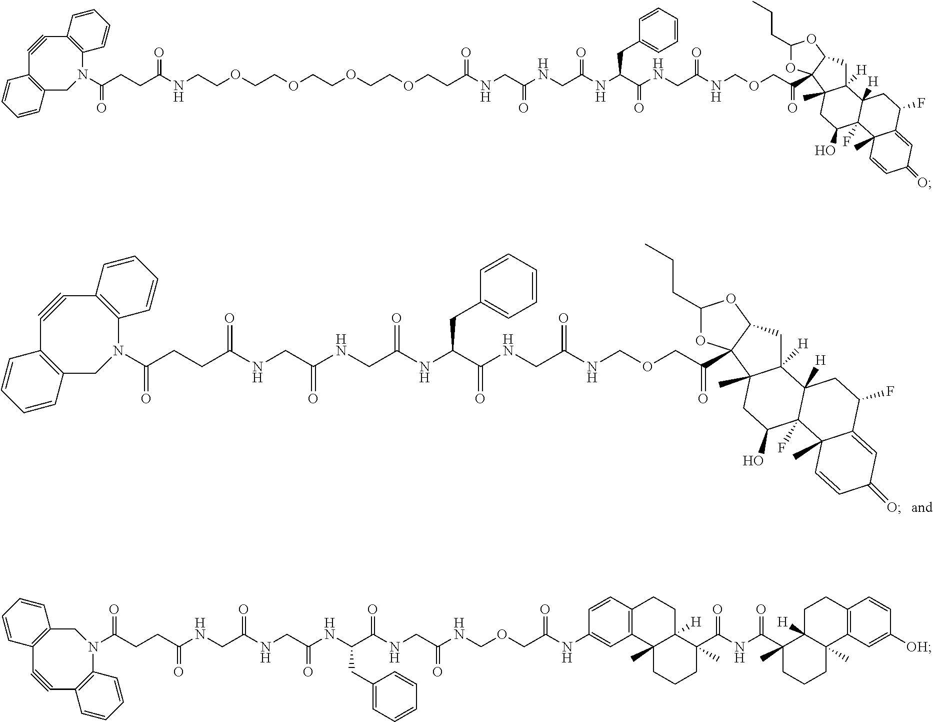
or a pharmaceutically acceptable salt thereof.

73. The compound of claim 41, wherein
R^{1a} and R^{1b} are hydrogen;
R³ is hydrogen; and
n is zero.

74. The compound of claim 73, wherein D* is a residue of a biologically active compound comprising hydroxyl.

75. The compound of claim 74, selected from the group consisting of 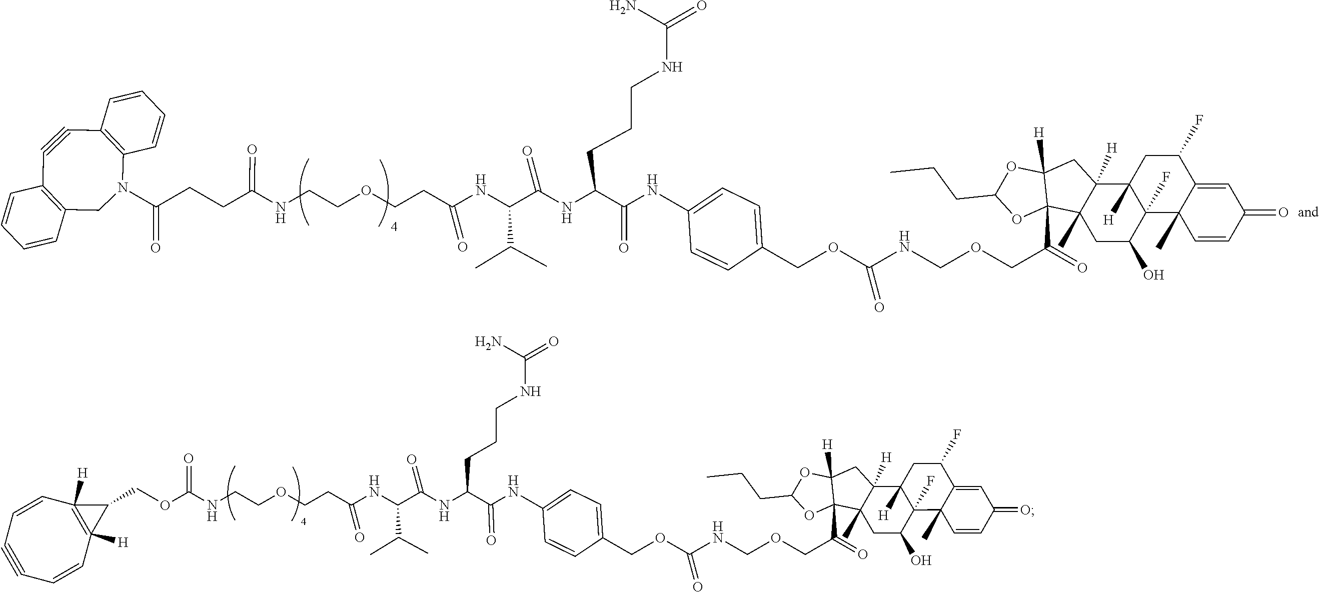
or a pharmaceutically acceptable salt thereof.

76. The compound of claim 57, wherein
R^{1a} and R^{1b} are hydrogen;
R² is hydrogen;
R³ is alkyl;
R⁴ is alkyl; and
n is one.

77. The compound of claim 76, wherein D* is a residue of a biologically active compound comprising hydroxyl.

78. The compound of claim 77, having the following structure 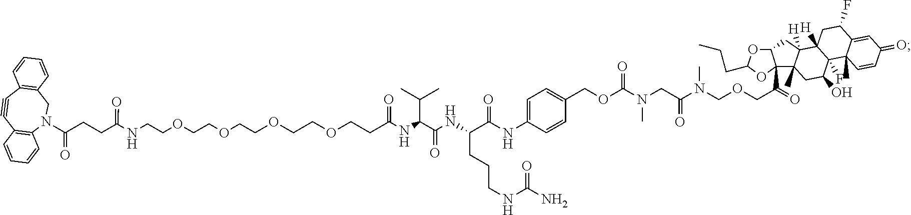 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCN(C)C(=O)CN(C)C(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)O1]
or a pharmaceutically acceptable salt thereof.

79. The compound of claim 39, wherein
R^{1a} and R^{1b} are hydrogen;
R² is hydrogen or —CH₂OH;
R³ is hydrogen; and
n is six.

80. The compound of claim 79, wherein D* is a residue of a biologically active compound comprising hydroxyl.

81. The compound of claim 80, having the following structure 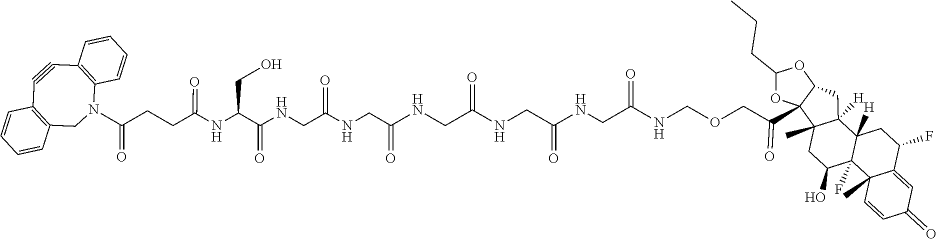 [SMILES: CCCC1O[C@@H]2C[C@H]3[C@@H]4C[C@H](F)C5=CC(=O)C=C[C@]5(C)[C@@]4(F)[C@@H](O)C[C@]3(C)[C@]2(C(=O)COCNC(=O)CNC(=O)CNC(=O)CNC(=O)CNC(=O)CNC(=O)[C@H](CO)NC(=O)CCC(=O)N2Cc3ccccc3C#Cc3ccccc32)O1]
or a pharmaceutically acceptable salt thereof.

82. The compound of claim 43, wherein
R^{1a} and R^{1b} are hydrogen;
R² is alkylene, wherein the alkylene is further bonded to R³ to form a 6-membered heterocyclyl;
R³ is alkylene, wherein the alkylene is further bonded to R² to form the 6-membered heterocyclyl; and
n is one.

83. The compound of claim 82, wherein D* is a residue of a biologically active compound comprising hydroxyl.

84. The compound of claim 83, wherein the compound is 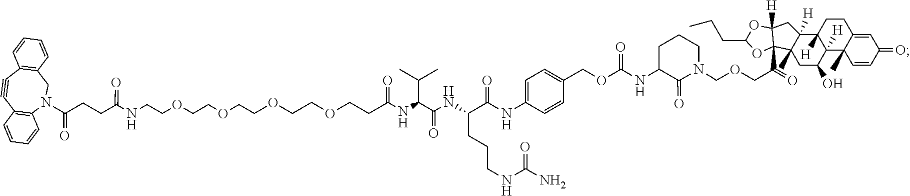
or a pharmaceutically acceptable salt thereof.

85. The compound of claim 53, wherein the compound is 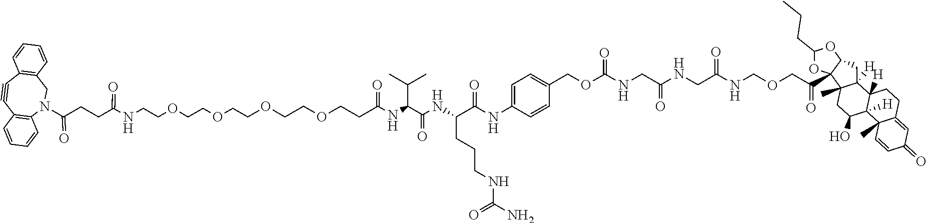
or a pharmaceutically acceptable salt thereof.

86. The compound of claim 53, wherein the compound is 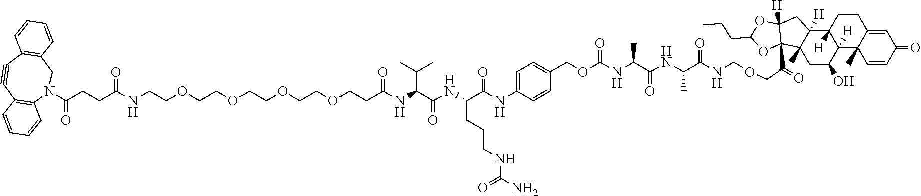
or a pharmaceutically acceptable salt thereof.

87. The compound of claim 65, wherein the compound is 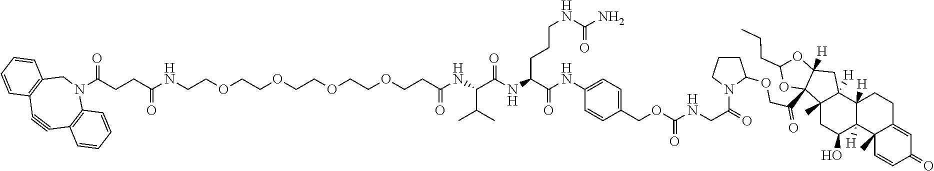 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCCN2C(=O)CNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)O1]
or a pharmaceutically acceptable salt thereof.

88. The compound of claim 56, wherein the compound is 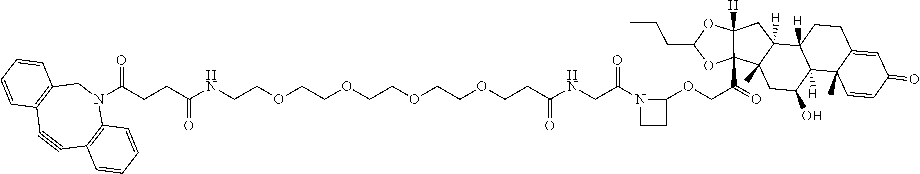 [SMILES: CCCC1O[C@@H]2C[C@H]3[C@@H]4CCC5=CC(=O)C=C[C@]5(C)[C@H]4[C@@H](O)C[C@]3(C)[C@]2(C(=O)COC2CCN2C(=O)CNC(=O)CCOCCOCCOCCOCCNC(=O)CCC(=O)N2Cc3ccccc3C#Cc3ccccc32)O1]
or a pharmaceutically acceptable salt thereof.

89. The compound of claim 56, wherein the compound is 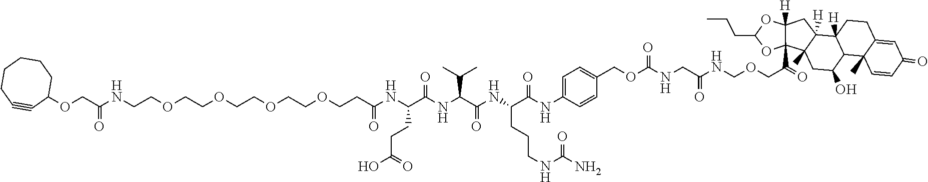 [SMILES: CCCC1O[C@@H]2C[C@H]3[C@@H]4CCC5=CC(=O)C=C[C@]5(C)C4[C@@H](O)C[C@]3(C)[C@]2(C(=O)COCNC(=O)CNC(=O)OCc2ccc(NC(=O)[C@H](CCCNC(N)=O)NC(=O)[C@@H](NC(=O)[C@H](CCC(=O)O)NC(=O)CCOCCOCCOCCOCCNC(=O)COC3C#CCCCCC3)C(C)C)cc2)O1]
or a pharmaceutically acceptable salt thereof.

90. A compound having the following structure 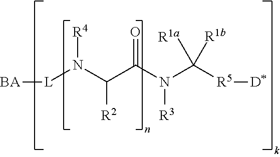
wherein
L is a linker;
BA is a binding agent;
R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 3-, 4-, 5-, 6-, 7-, or 8-membered heterocyclyl;
R² is hydrogen, alkylene, heteroalkylene, or an amino acid side chain, wherein when R² is alkylene or heteroalkylene, the alkylene or heteroalkylene is further bonded to R³ to form a 4-, 5, or 6-membered heterocyclyl;
R³ is hydrogen, alkyl, alkylene, or heteroalkylene, wherein when R³ is alkylene or heteroalkylene, the alkylene or heteroalkylene is further bonded to R^{1a} or R² to form the 3-, 4-, 5-, 6-, 7-, or 8-membered heterocyclyl;
R⁴ is hydrogen or alkyl;
R⁵ is oxygen, NR⁶, or sulfur;
R⁶ is hydrogen or alkyl;
D* is a residue of a biologically active compound comprising hydroxyl, amino, or thiol;
n is zero, one, two, three, four, five, or six; and
k is an integer from one to thirty.

91. The compound of claim 90, wherein D* is a residue of an anti-inflammatory biologically active compound comprising hydroxyl, amino, or thiol.

92. The compound of claim 91, wherein the anti-inflammatory biologically active compound is a steroid or a residue thereof.

93. The compound of claim 91, wherein the anti-inflammatory biologically active compound is an LXR agonist or a residue thereof.

94. A compound having the following structure 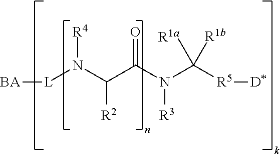
wherein
L is a linker;
BA is a binding agent;
R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 3-, 4-, 5-, 6-, 7-, or 8-membered heterocyclyl;
R² is hydrogen, alkylene, heteroalkylene, or an amino acid side chain, wherein when R² is alkylene or heteroalkylene, the alkylene or heteroalkylene is further bonded to R³ to form a 4-, 5, or 6-membered heterocyclyl;
R³ is hydrogen, alkyl, alkylene, or heteroalkylene, wherein when R³ is alkylene or heteroalkylene, the alkylene or heteroalkylene is further bonded to R^{1a} or R² to form the 3-, 4-, 5-, 6-, 7-, or 8-membered heterocyclyl;
R⁴ is hydrogen or alkyl;
R⁵ is oxygen, NR⁶, or sulfur;
R⁶ is hydrogen or alkyl;
D* is a residue of an anti-inflammatory biologically active compound comprising hydroxyl, amino, or thiol;
n is zero, one, two, three, four, five, or six; and
k is an integer from one to thirty.

95. The compound of claim 94, wherein D* is a residue of a steroid comprising hydroxyl, amino, or thiol.

96. A compound having the following structure 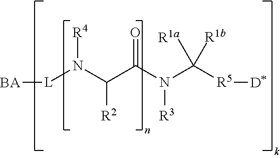
wherein
L is a linker comprising 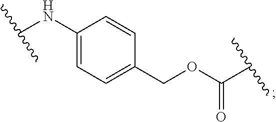 [SMILES: *Nc1ccc(COC(*)=O)cc1]
BA is a binding agent;
R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 3-, 4-, 5-, 6-, 7-, or 8-membered heterocyclyl;
R² is hydrogen, alkylene, heteroalkylene, or an amino acid side chain, wherein when R² is alkylene or heteroalkylene, the alkylene or heteroalkylene is further bonded to R³ to form a 4-, 5, or 6-membered heterocyclyl;
R³ is hydrogen, alkyl, alkylene, or heteroalkylene, wherein when R³ is alkylene or heteroalkylene, the alkylene or heteroalkylene is further bonded to R^{1a} or R² to form the 3-, 4-, 5-, 6-, 7-, or 8-membered heterocyclyl;
R⁴ is hydrogen or alkyl;
R⁵ is oxygen, NR⁶, or sulfur;
R⁶ is hydrogen or alkyl;
D* is a residue of a biologically active compound comprising hydroxyl, amino, or thiol;
n is zero, one, two, three, four, five, or six; and
k is an integer from one to thirty.

97. The compound of claim 96, wherein L is a linker comprising 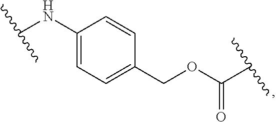 [SMILES: *Nc1ccc(COC(*)=O)cc1]
and R² is hydrogen.

98. A compound having the following structure 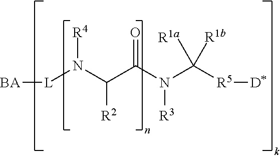
wherein
L is a linker;
BA is a binding agent;
R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 3-, 4-, 5-, 6-, 7-, or 8-membered heterocyclyl;
R² is hydrogen, alkylene, heteroalkylene, or an amino acid side chain, wherein when R² is alkylene or heteroalkylene, the alkylene or heteroalkylene is further bonded to R³ to form a 4-, 5, or 6-membered heterocyclyl;
R³ is hydrogen, alkyl, alkylene, or heteroalkylene, wherein when R³ is alkylene or heteroalkylene, the alkylene or heteroalkylene is further bonded to R^{1a} or R² to form the 3-, 4-, 5-, 6-, 7-, or 8-membered heterocyclyl;
R⁴ is hydrogen or alkyl;
R⁵ is oxygen, NR⁶, or sulfur;
R⁶ is hydrogen or alkyl;
D* is a residue of a biologically active compound comprising hydroxyl, amino, or thiol;
n is zero, one, two, three, four, five, or six;
wherein conjugation of L to BA is selected from the group consisting of a click chemistry residue, an amide residue, and a residue comprising two cysteine residues of a single BA that are chemically bonded to L; and
k is an integer from one to thirty.

99. The compound of claim 90, having the following structure 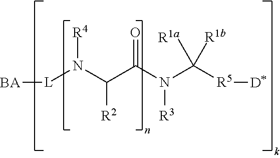
wherein
L is a linker;
BA is a binding agent;
R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl;
R² is hydrogen, alkylene, heteroalkylene, or an amino acid side chain, wherein when R² is alkylene or heteroalkylene, the alkylene or heteroalkylene is further bonded to R³ to form a 4-, 5, or 6-membered heterocyclyl;
R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl;
R⁴ is hydrogen or alkyl;
R⁵ is oxygen, NR⁶, or sulfur;
R⁶ is hydrogen or alkyl;
D* is a residue of a biologically active compound comprising hydroxyl, amino, or thiol;
n is zero, one, two, three, four, five, or six; and
k is an integer from one to thirty.

100. The compound of claim 99 having the structure 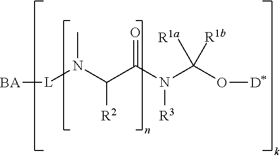
wherein
L is a linker;
BA is a binding agent;
R^{1a} and R^{1b} are, independently, hydrogen, alkyl, alkoxy, alkenyl, alkynyl, aryl, arylalkyl, heteroaryl, or alkylene, wherein when R^{1a} is alkylene, the alkylene is further bonded to R³ to form a 4-, 5-, or 6-membered heterocyclyl;
R² is hydrogen or an amino acid side chain;
R³ is hydrogen, alkyl, or alkylene, wherein when R³ is alkylene, the alkylene is further bonded to R^{1a} to form the 4-, 5-, or 6-membered heterocyclyl;
D* is a residue of a biologically active compound comprising hydroxyl;
n is zero, one, two, three, four, five, or six; and
k is an integer from one to thirty.

101. The compound of claim 99 having the following structure 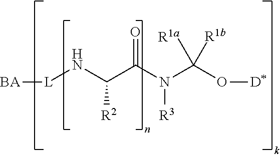
wherein
L is a linker;
BA is a binding agent;
R^{1a} and R^{1b} are, independently, hydrogen, alkyl, alkoxy, alkenyl, alkynyl, aryl, arylalkyl, heteroaryl, or alkylene, wherein when R^{1a} is alkylene, the alkylene is further bonded to R³ to form a 4-, 5-, or 6-membered heterocyclyl;
R² is hydrogen or an amino acid side chain;
R³ is hydrogen, alkyl, or alkylene, wherein when R³ is alkylene, the alkylene is further bonded to R^{1a} to form the 4-, 5-, or 6-membered heterocyclyl;
D* is a residue of a biologically active compound comprising hydroxyl;
n is zero, one, two, three, four, five, or six; and
k is an integer from one to thirty.

102. The compound of any one of claims 90-101, wherein the linker comprises

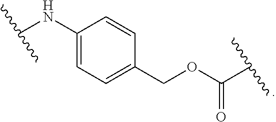 [SMILES: *Nc1ccc(COC(*)=O)cc1]

103. The compound of claim 99, wherein the compound is 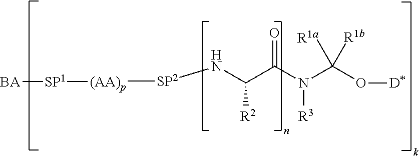
wherein
SP¹ and SP², when present, are spacer groups wherein SP¹ further comprises a moiety reactive with an antibody or an antigen binding fragment thereof;
each AA is an amino acid; and
p is an integer from 1 to 10.

104. The compound of claim 103, wherein SP¹ comprises a reactive group that comprises an alkyne.

105. The compound of claim 104, wherein the alkyne is capable of participating in a 1,3-cycloaddition reaction.

106. The compound of claim 105, wherein the alkyne is capable of participating in a 1,3-cycloaddition reaction with the binding agent, wherein the binding agent is an azido-functionalized binding agent.

107. The compound of claim 103, wherein SP² comprises

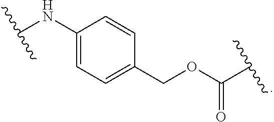 [SMILES: *Nc1ccc(COC(*)=O)cc1]

108. The compound of claim 103, wherein SP² comprises 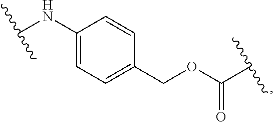 [SMILES: *Nc1ccc(COC(*)=O)cc1]
and R² is hydrogen or alkyl.

109. The compound of claim 106, wherein the alkyne is capable of participating in a 1,3-cycloaddition reaction with the binding agent, the binding agent is an azido-functionalized binding agent, and SP² comprises

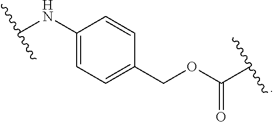 [SMILES: *Nc1ccc(COC(*)=O)cc1]

110. The compound of claim 106, wherein the alkyne is capable of participating in a 1,3-cycloaddition reaction with the binding agent, the binding agent is an azido-functionalized binding agent, SP² comprises 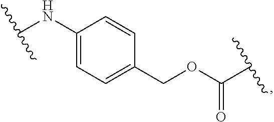 [SMILES: *Nc1ccc(COC(*)=O)cc1]
and R² is hydrogen or alkyl.

111. The compound of claim 103, wherein the binding agent is an antibody modified with a primary amine compound according to the Formula H₂N-LL-X, wherein LL is a divalent linker selected from the group consisting of
a divalent polyethylene glycol (PEG) group;
—(CH₂)ₙ—;
—(CH₂CH₂O)ₙ—(CH₂)ₚ—;
—(CH₂)ₙ—N(H)C(O)—(CH₂)ₘ—;
—(CH₂CH₂O)ₙ—N(H)C(O)—(CH₂CH₂O)ₘ—(CH₂)ₚ—;
—(CH₂)ₙ—C(O)N(H)—(CH₂)ₘ—;
—(CH₂CH₂O)ₙ—C(O)N(H)—(CH₂CH₂O)ₘ—(CH₂)ₚ—;
—(CH₂)ₙ—N(H)C(O)—(CH₂CH₂O)ₘ—(CH₂)ₚ—;
—(CH₂CH₂O)ₙ—N(H)C(O)—(CH₂)ₘ—;
—(CH₂)ₙ—C(O)N(H)—(CH₂CH₂O)ₘ—(CH₂)ₚ—; and
—(CH₂CH₂O)ₙ—C(O)N(H)—(CH₂)ₘ—,
wherein
n is an integer selected from 1 to 12;
m is an integer selected from 0 to 12;
p is an integer selected from 0 to 2; and
X is selected from the group consisting of —SH, —N₃, —C≡CH, —C(O)H, tetrazole,

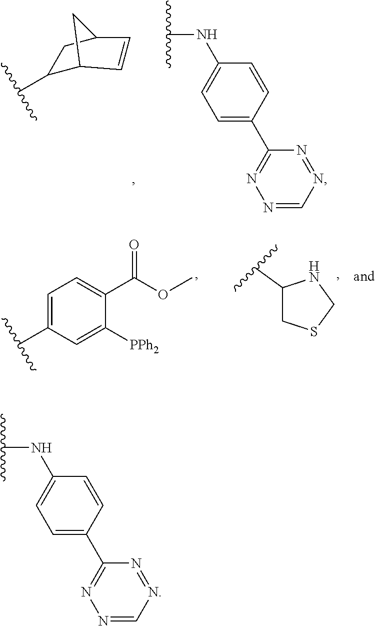

112. The compound of claim 111, wherein the binding agent is an antibody modified with a primary amine having the following structure

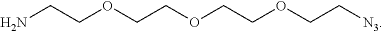 [SMILES: [N-]=[N+]=NCCOCCOCCOCCN]

113. The compound of claim 112, wherein the compound is selected from the group consisting of 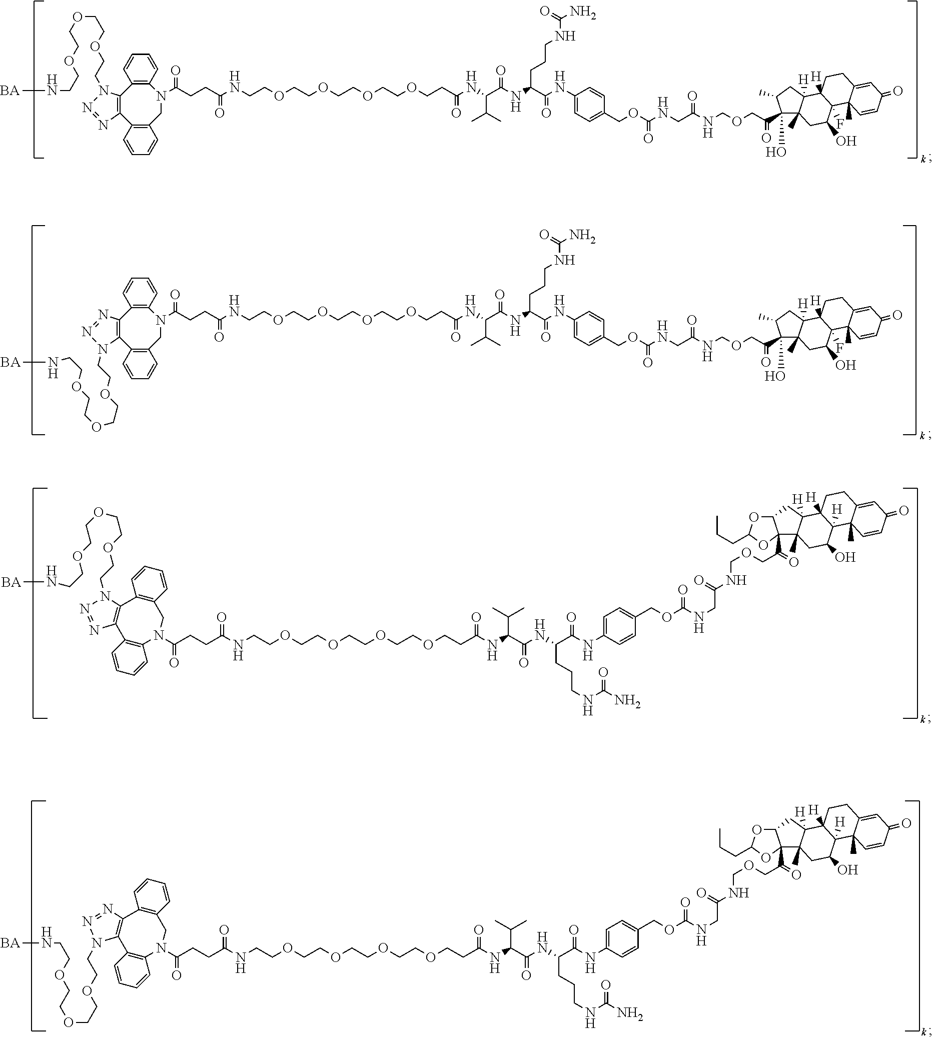 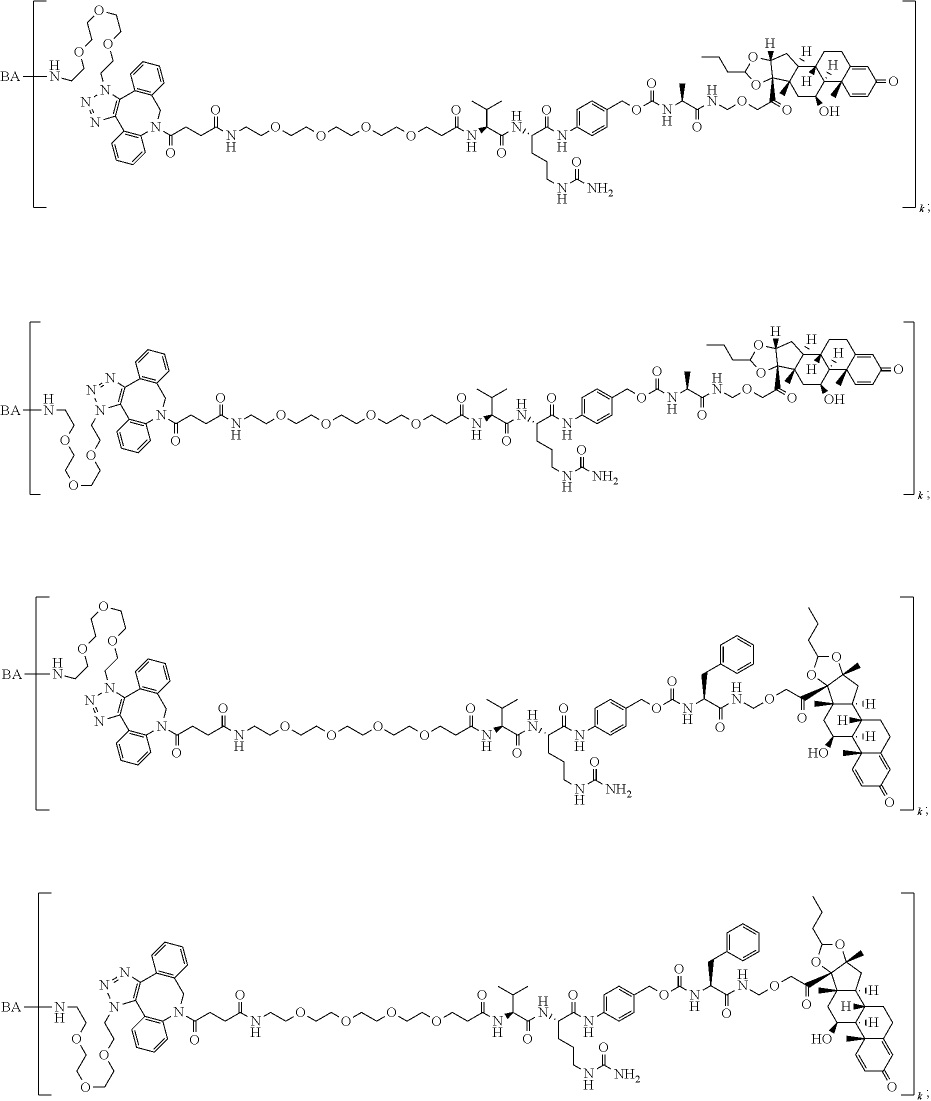 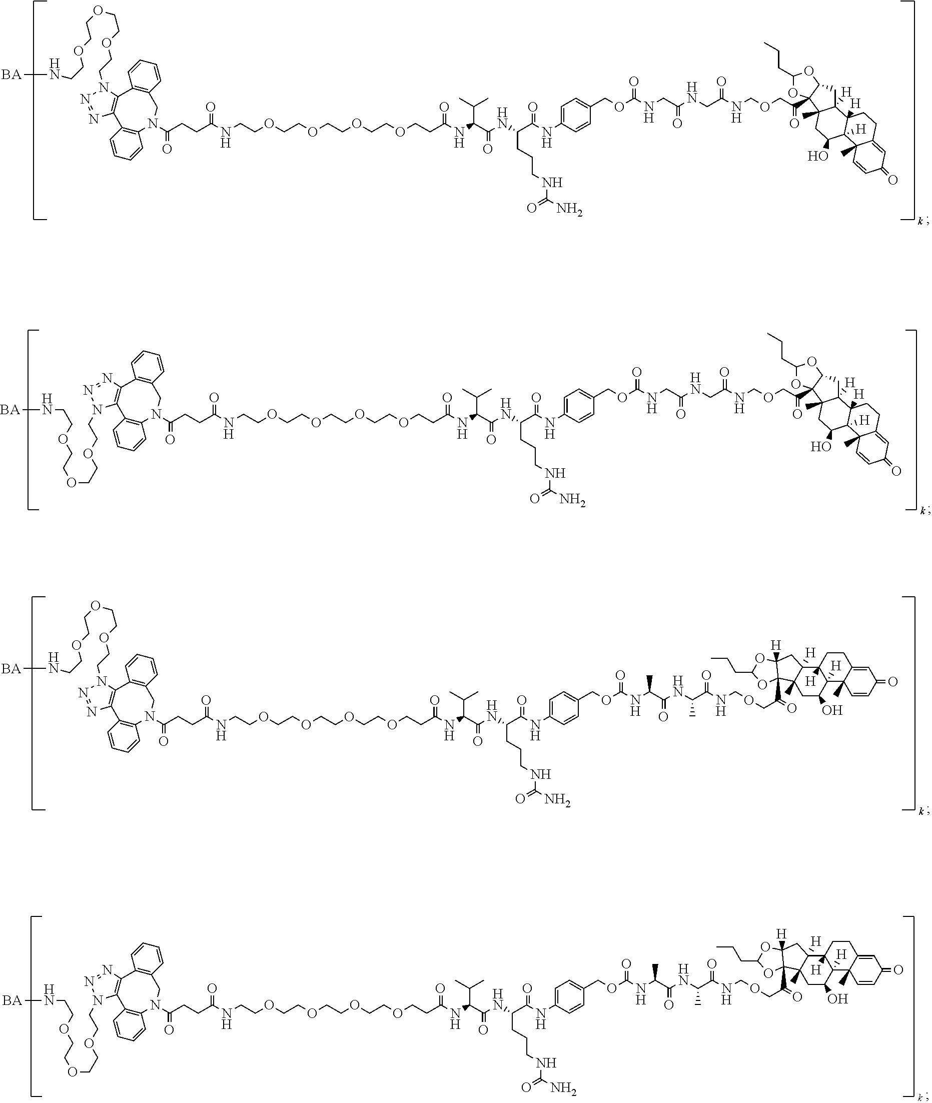 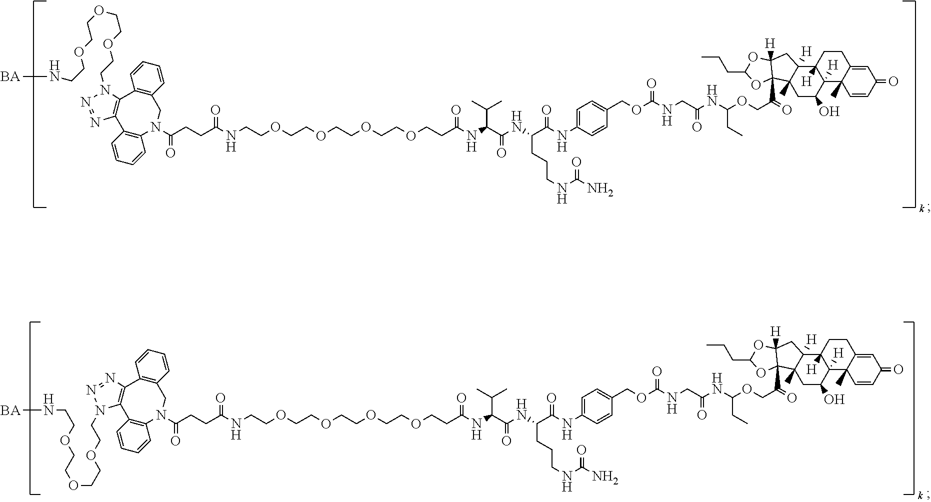 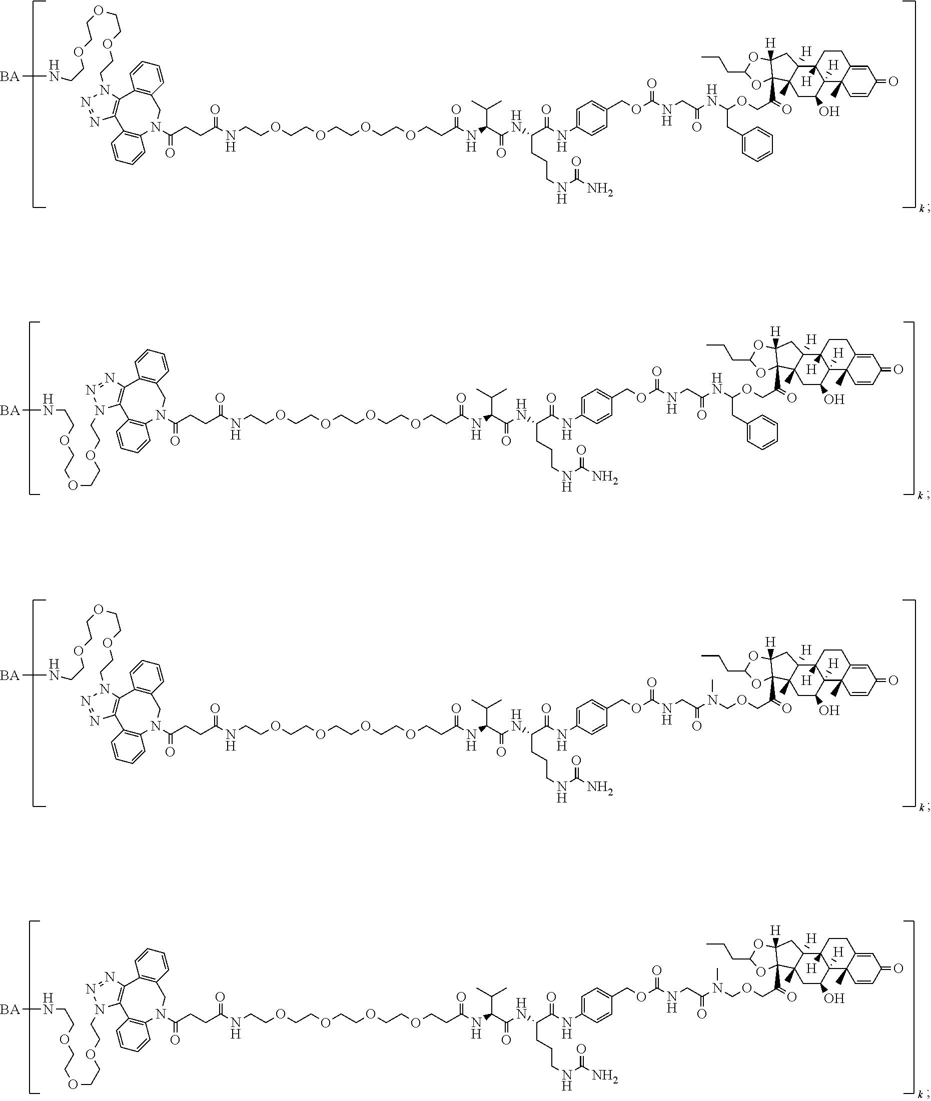 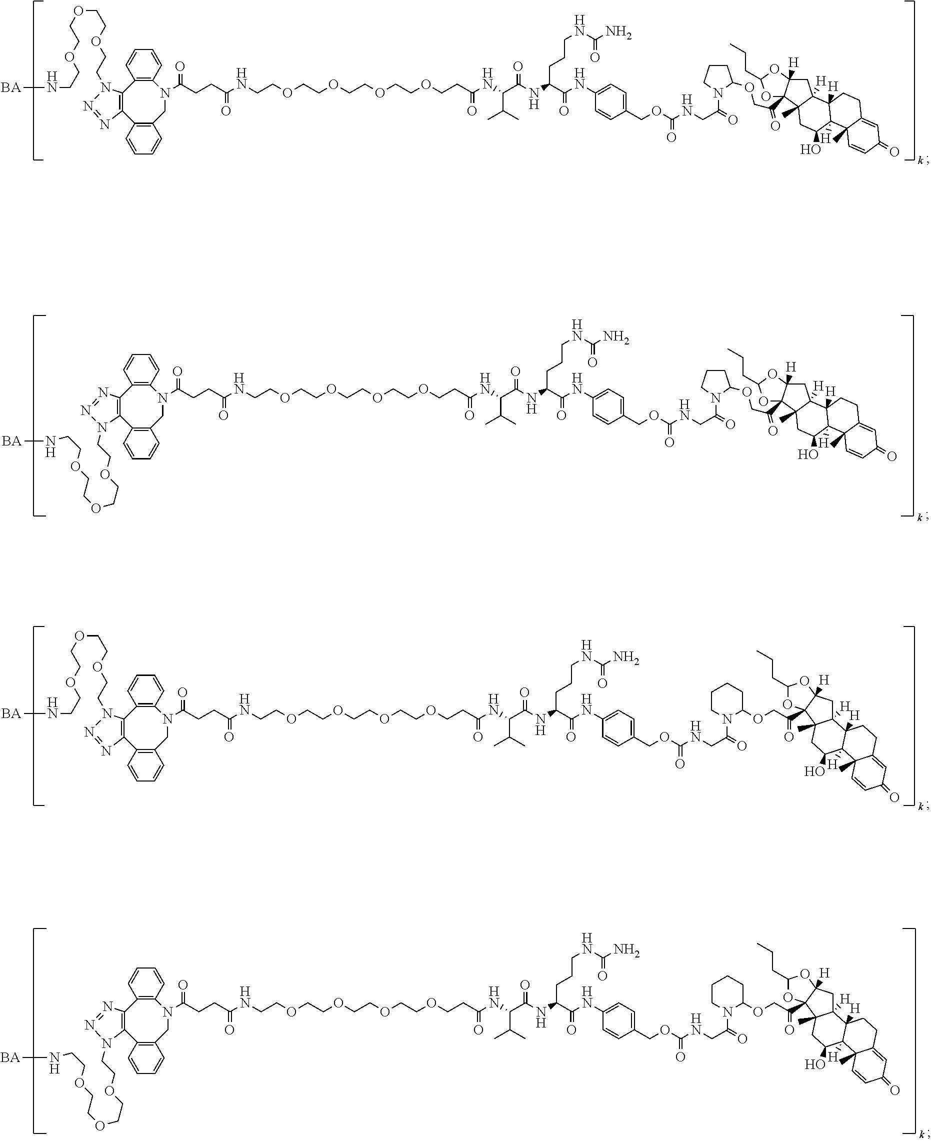 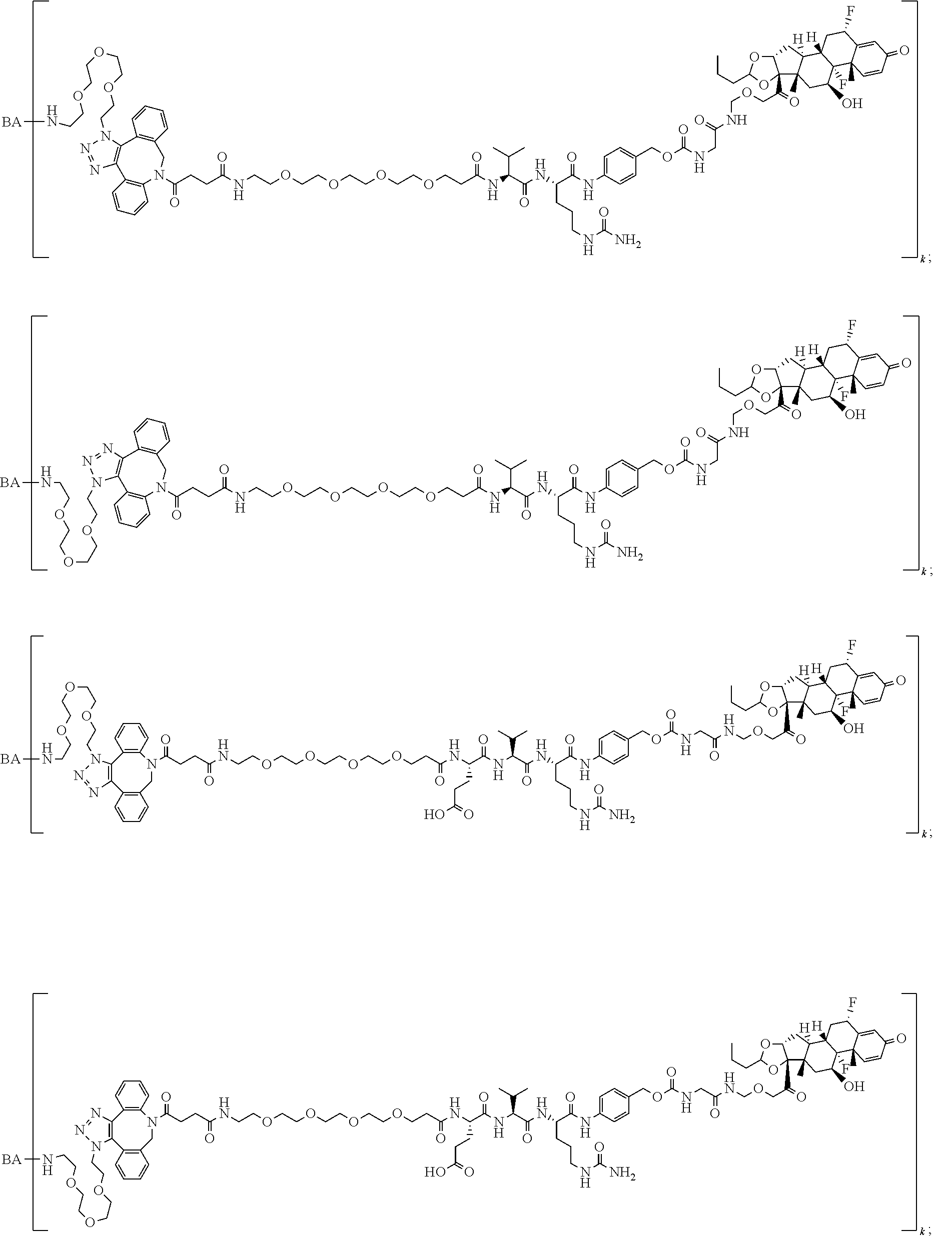 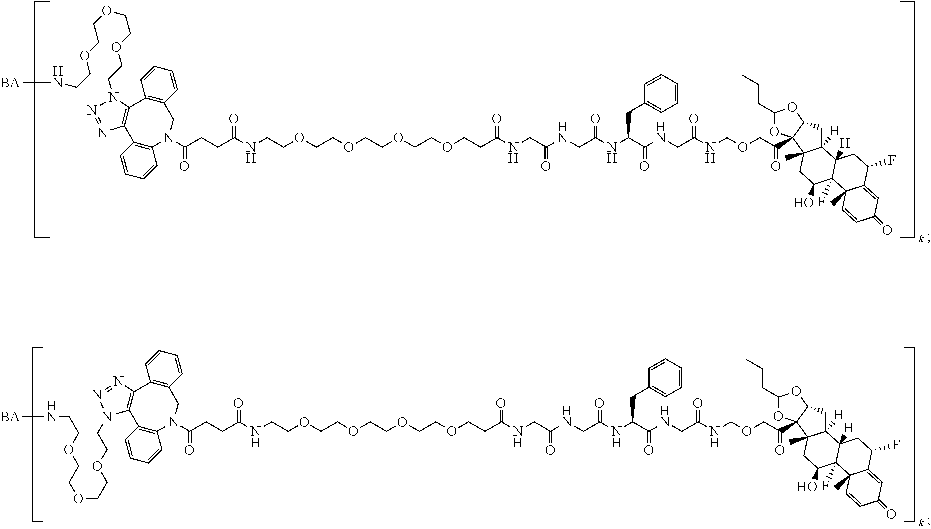 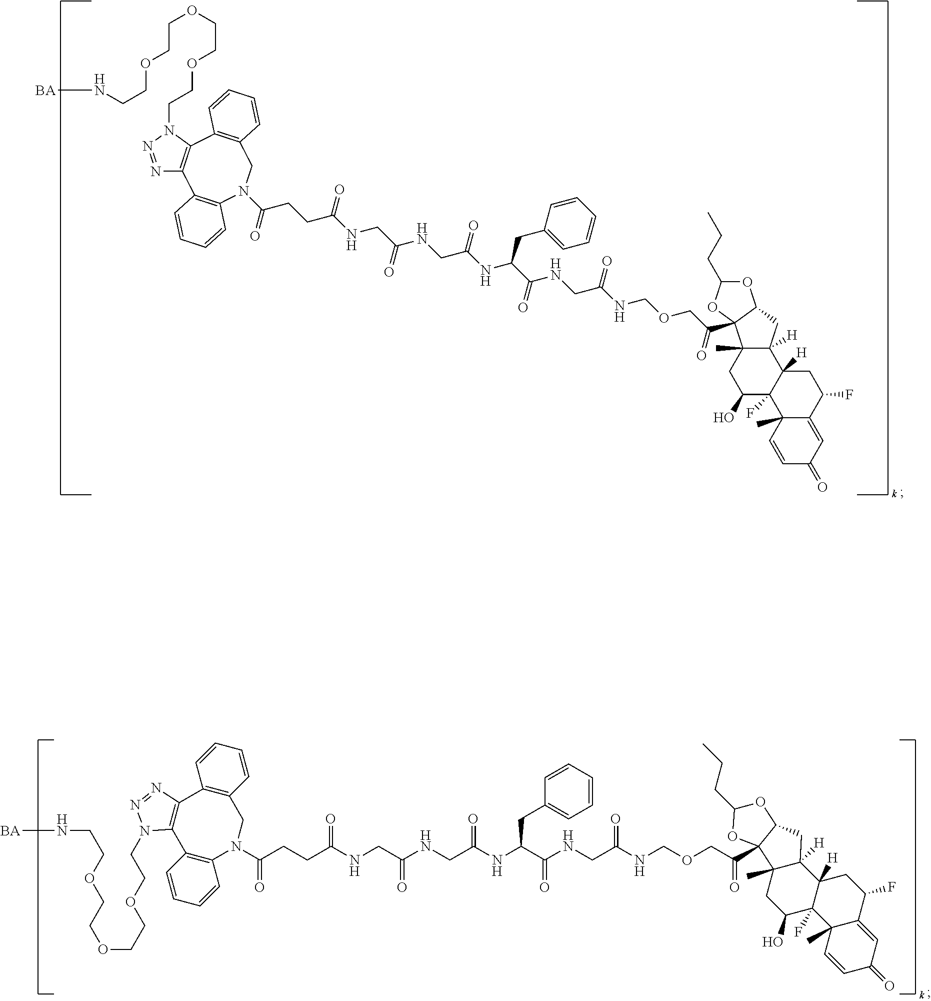 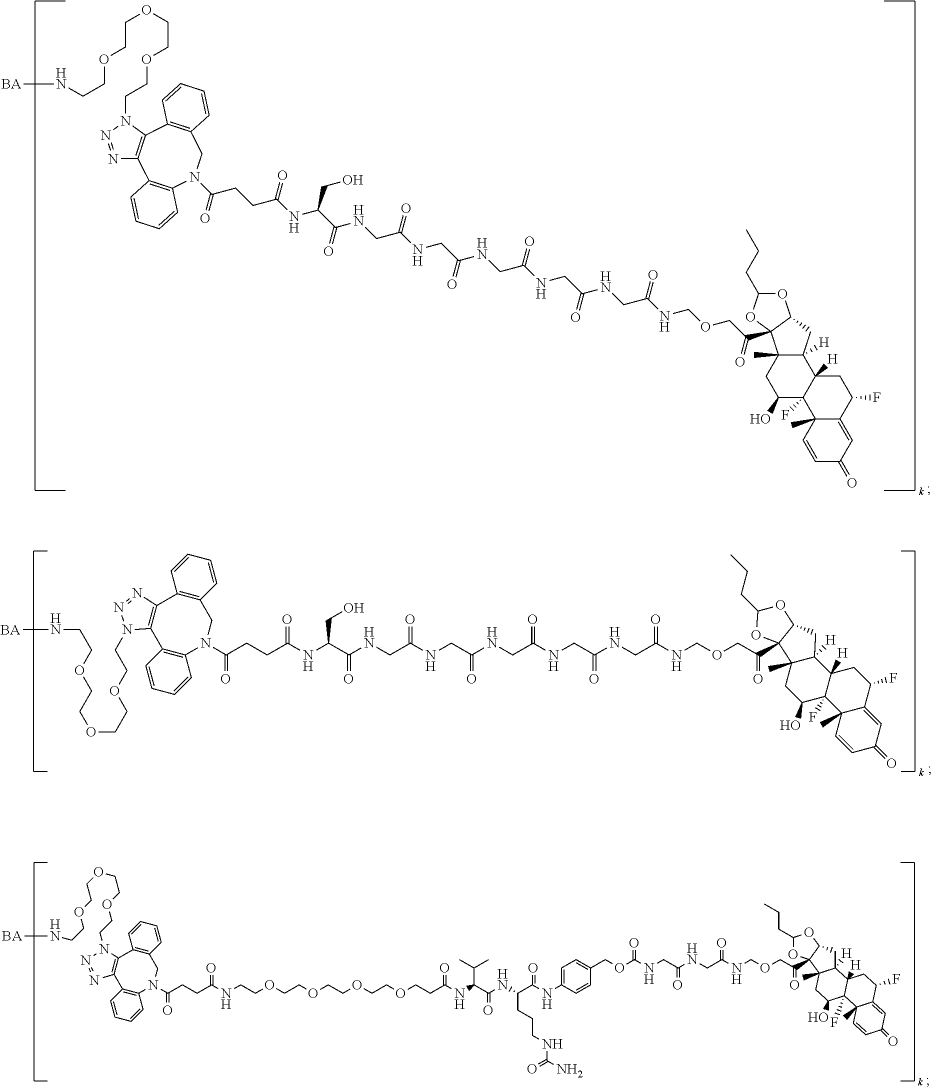 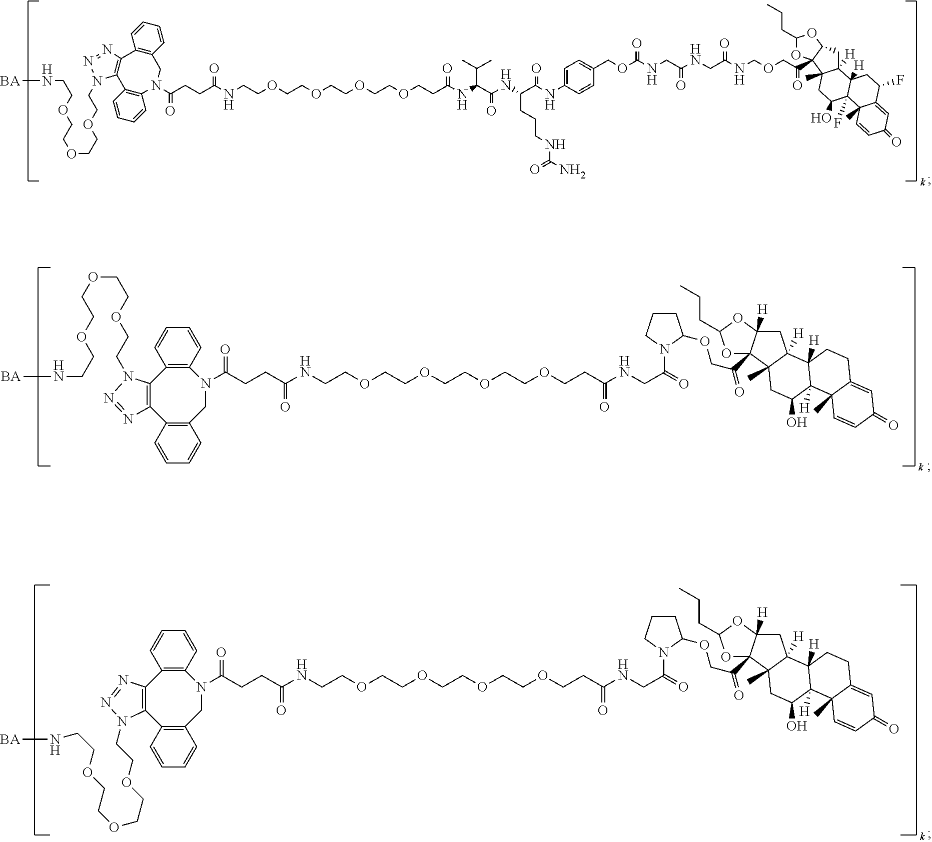 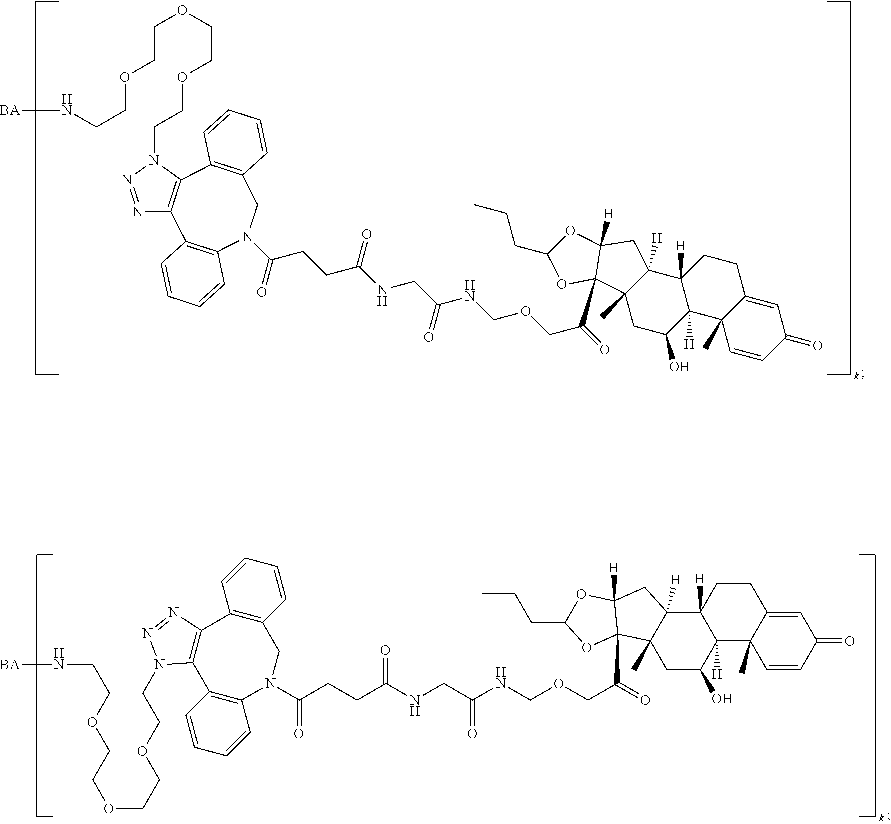 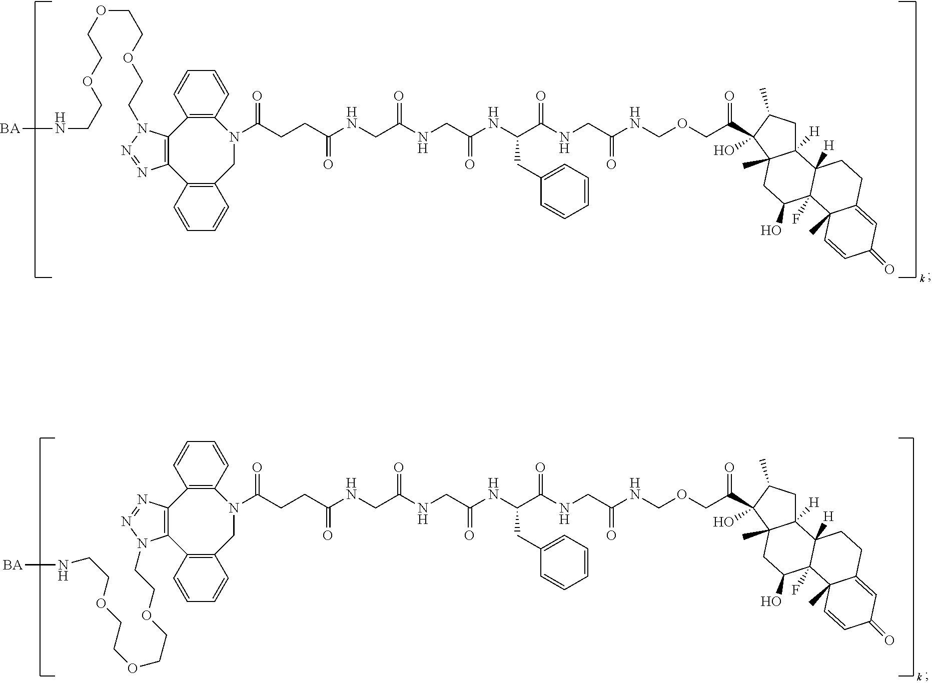 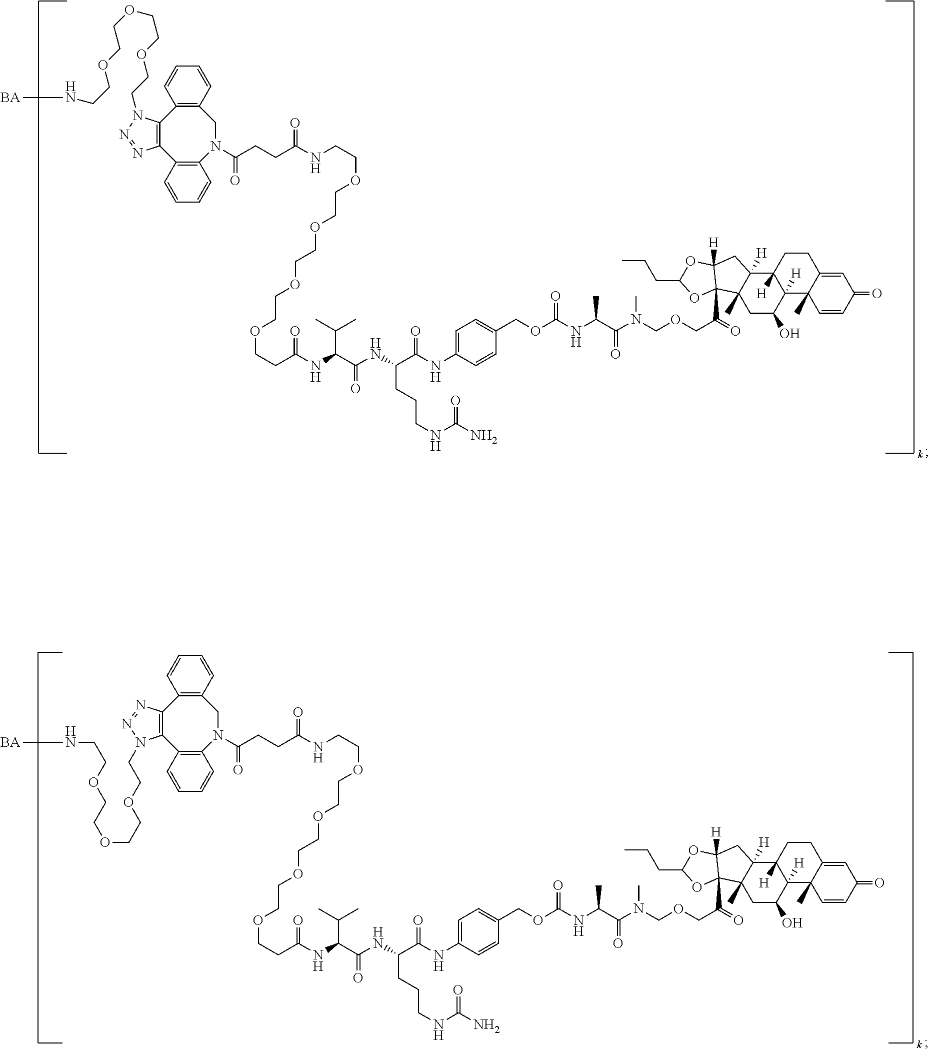 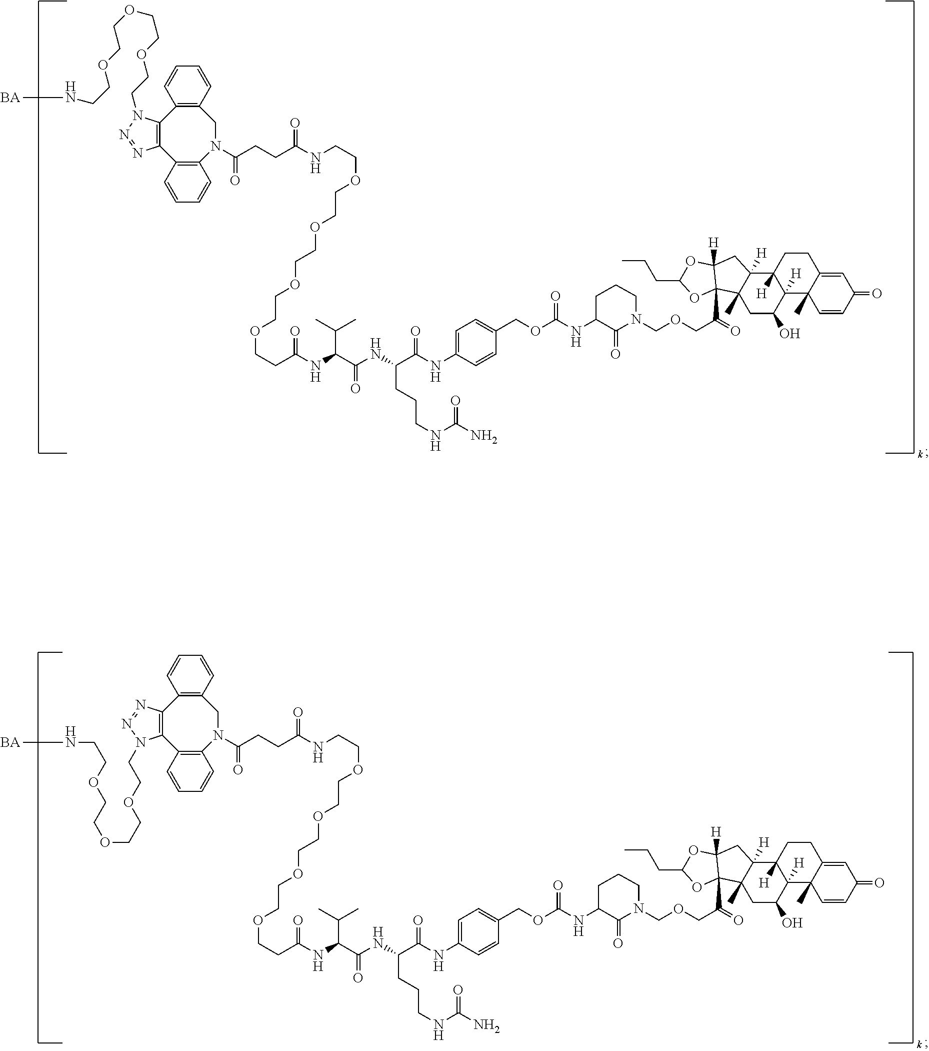 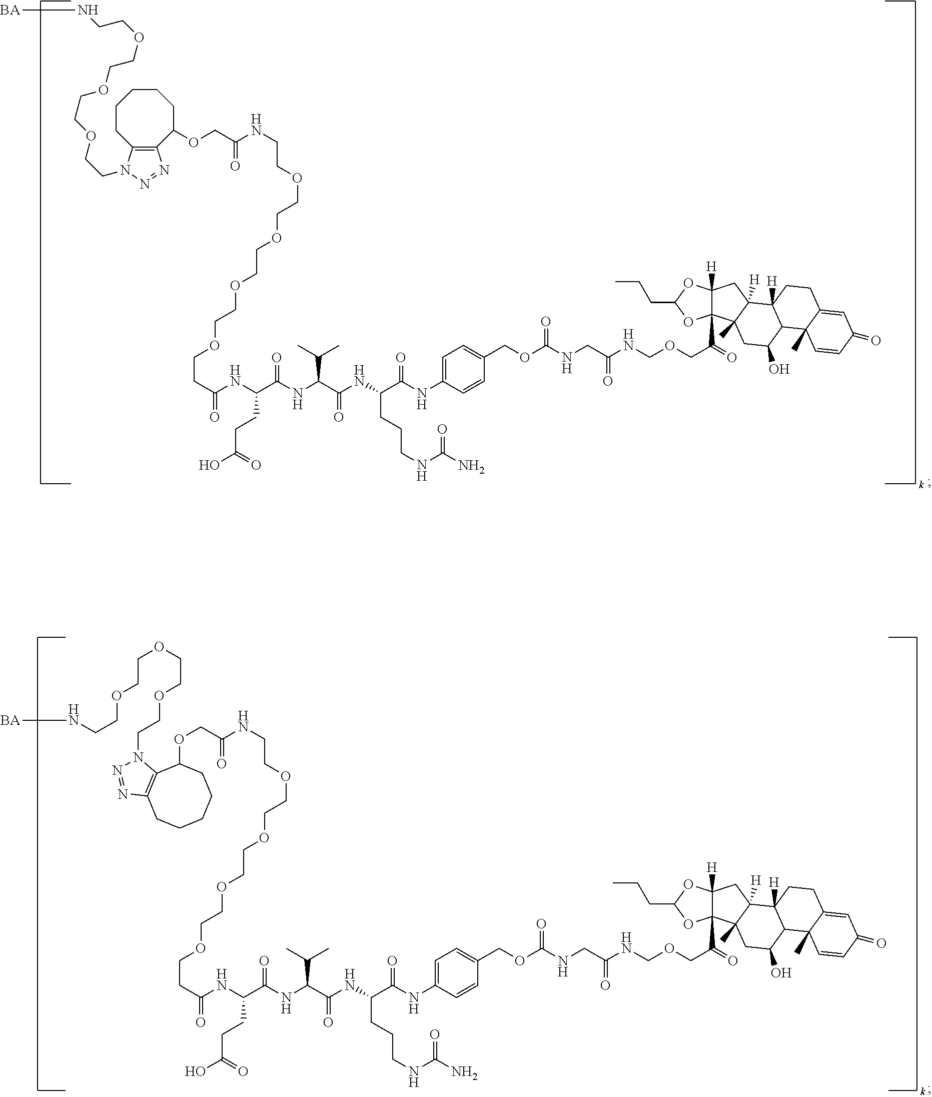 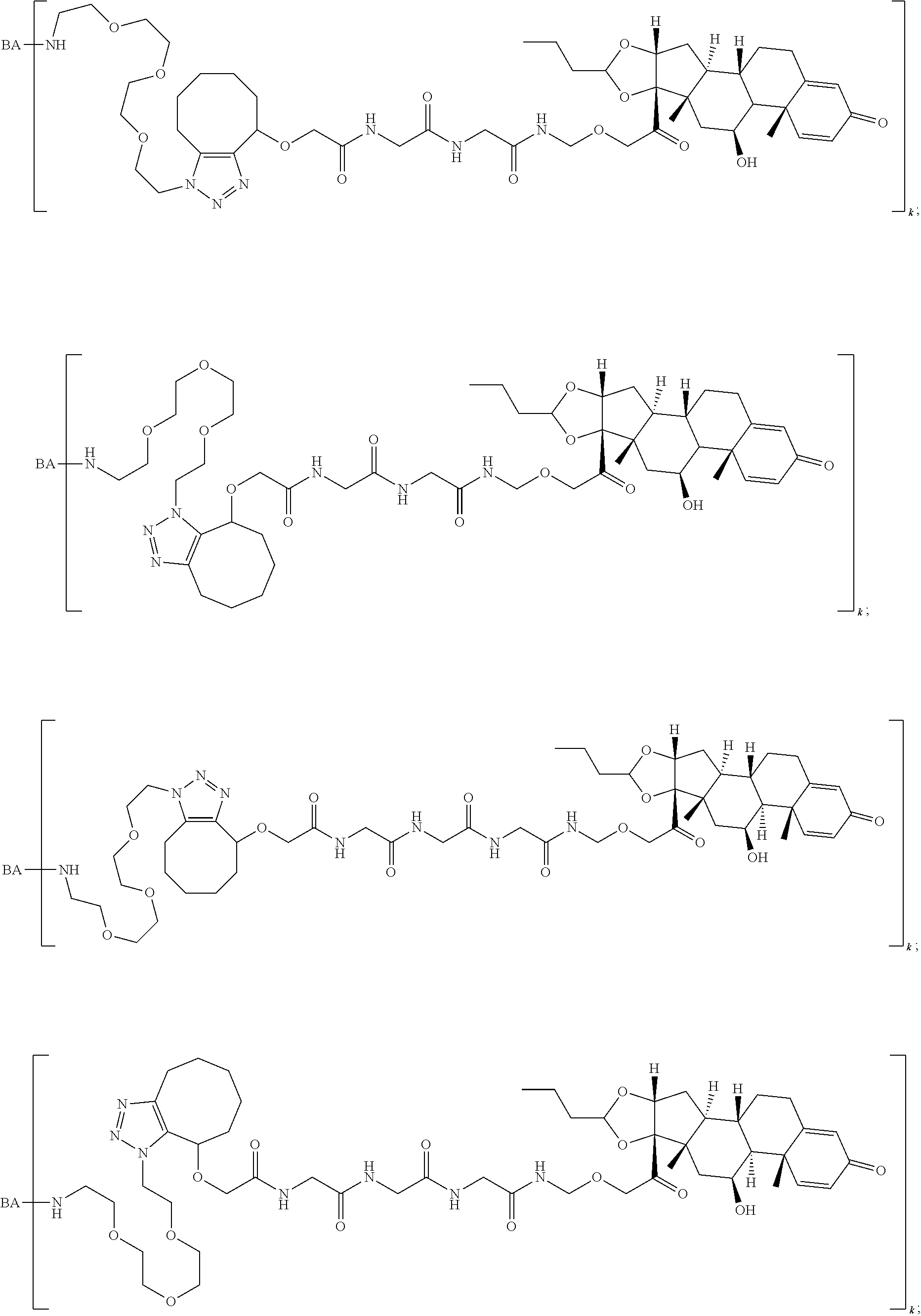 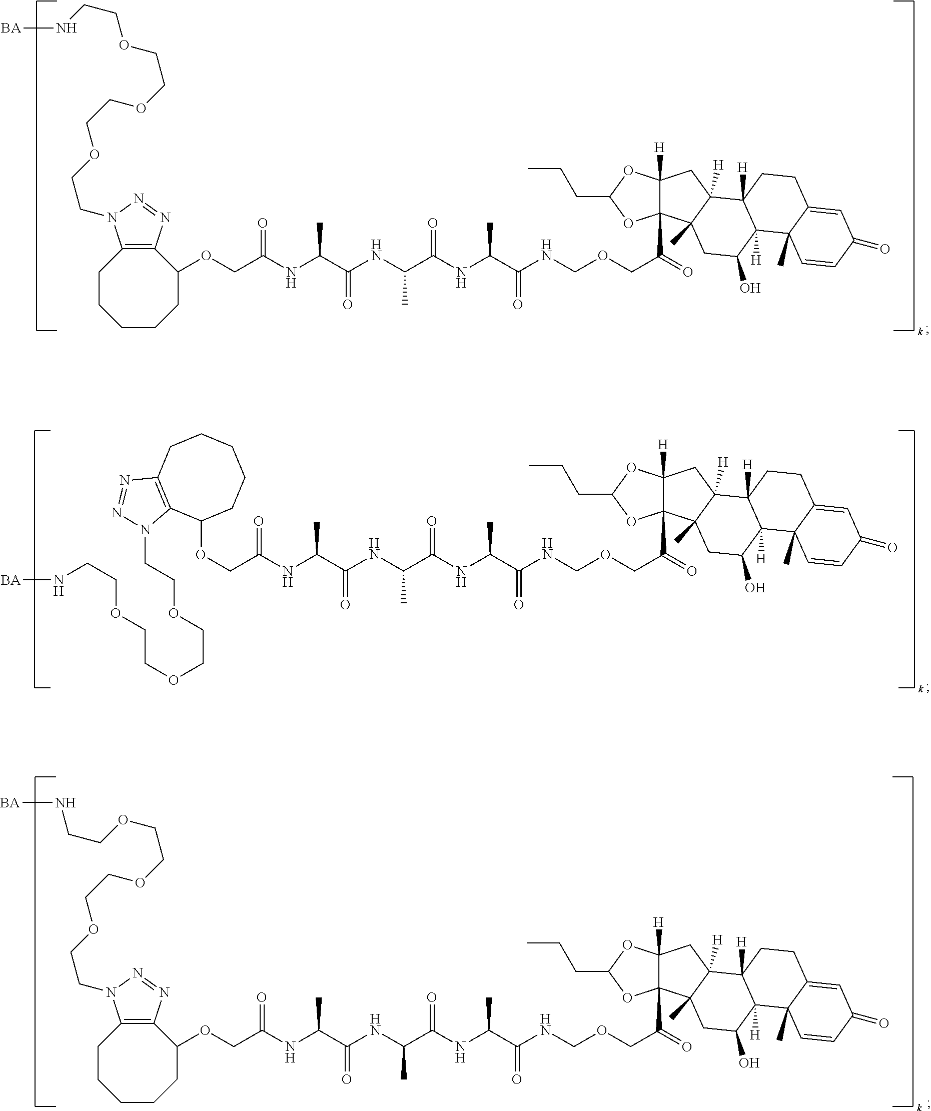 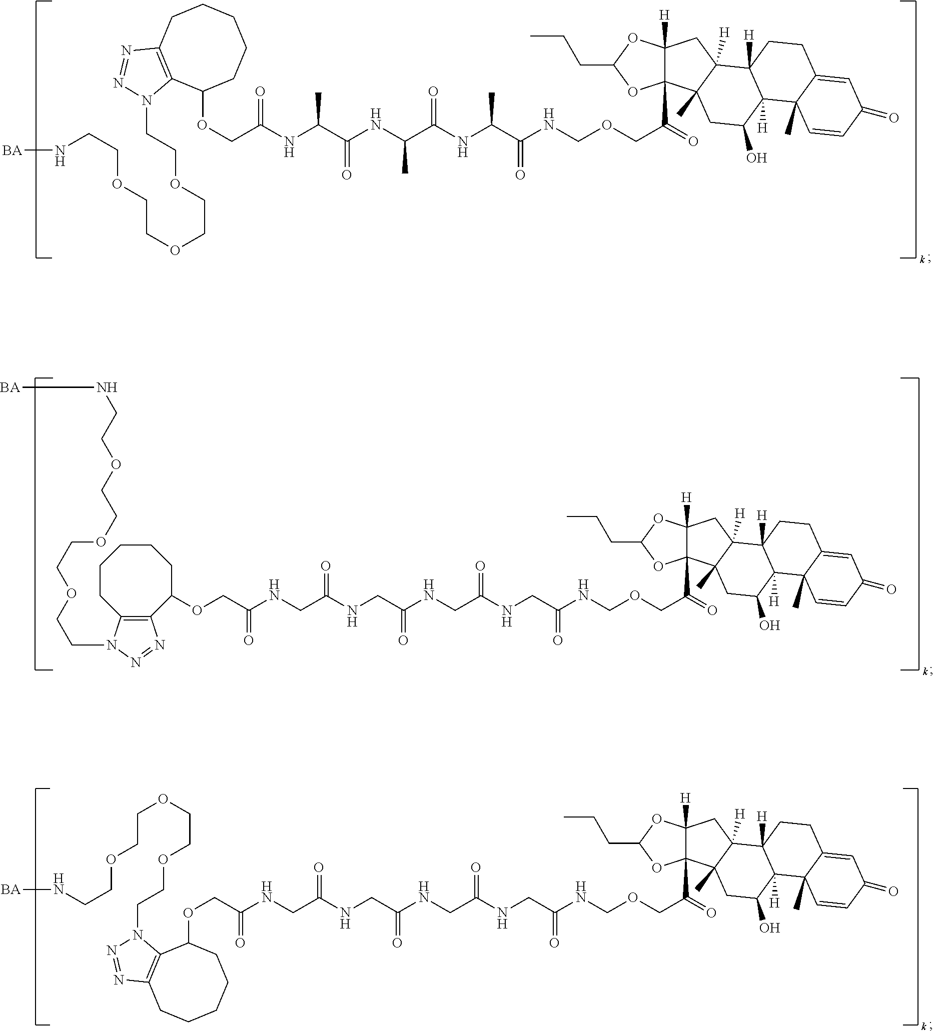 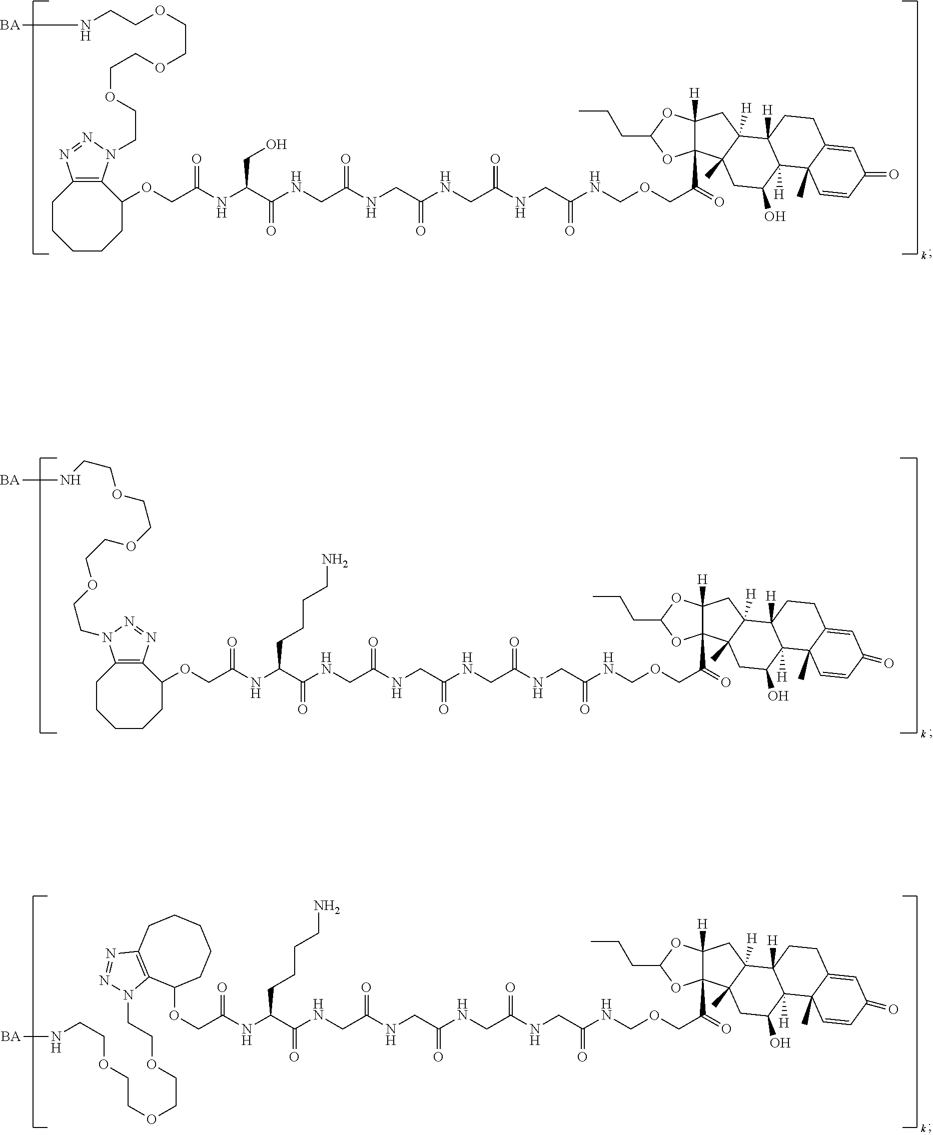 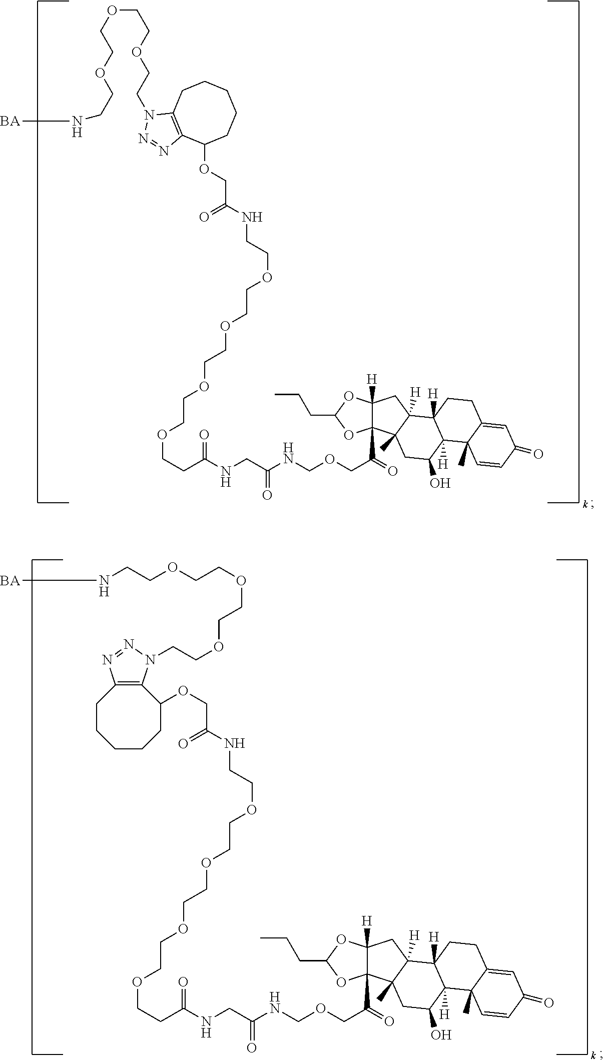 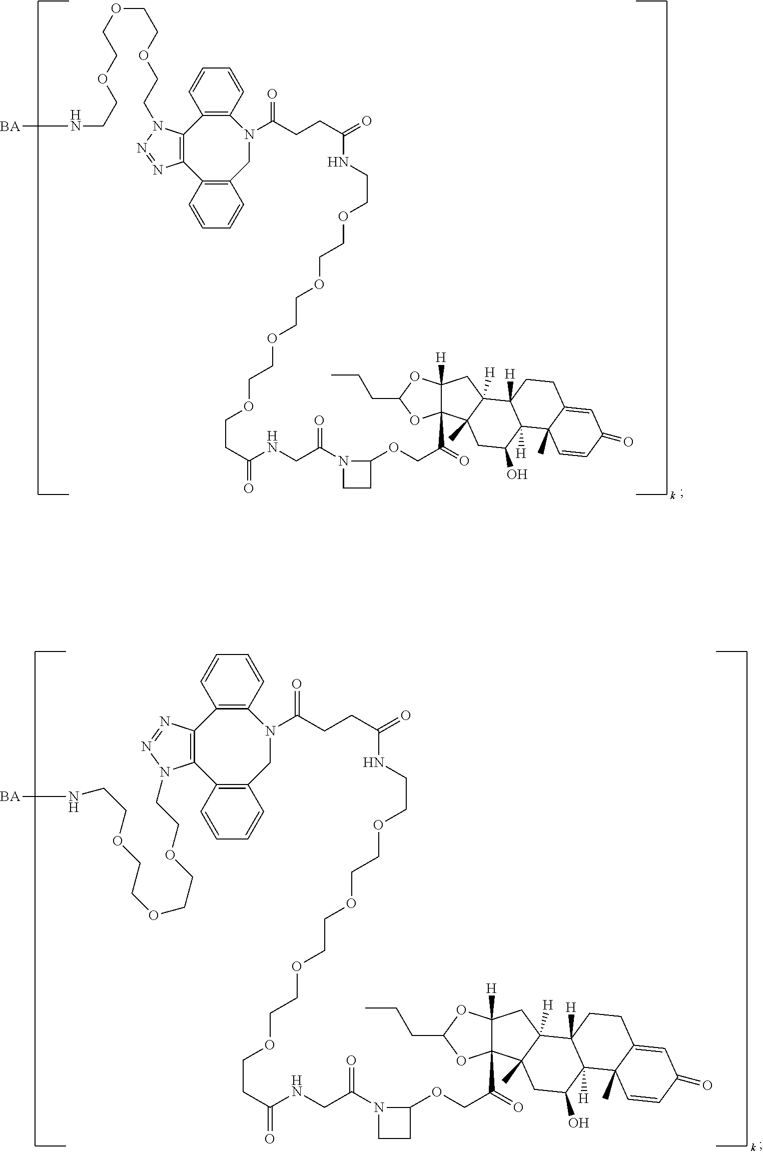 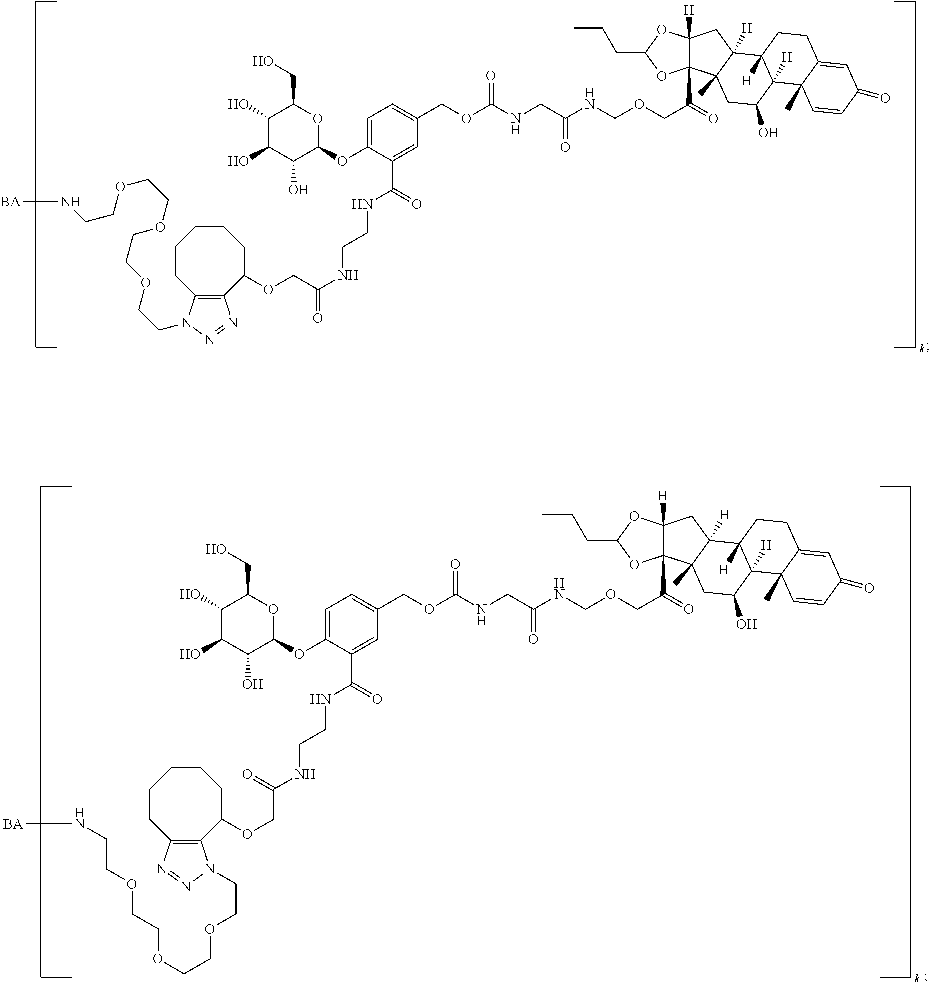 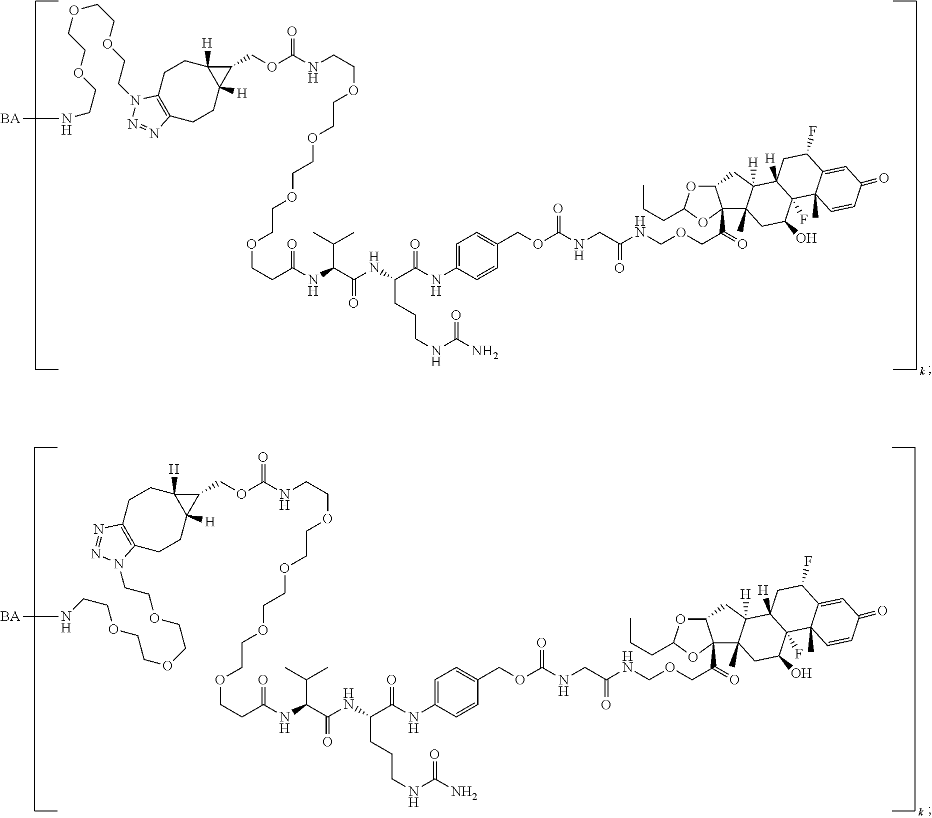 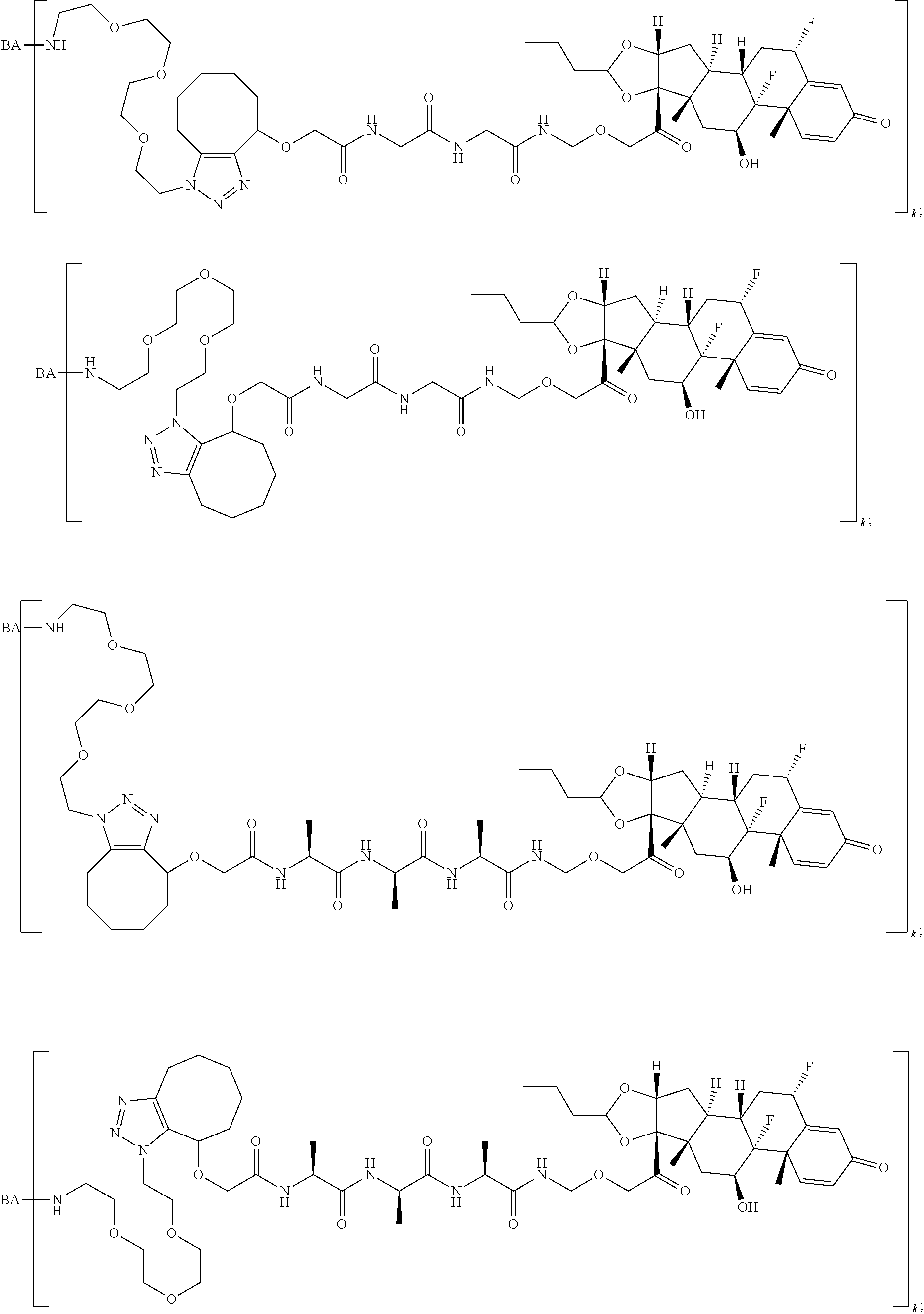 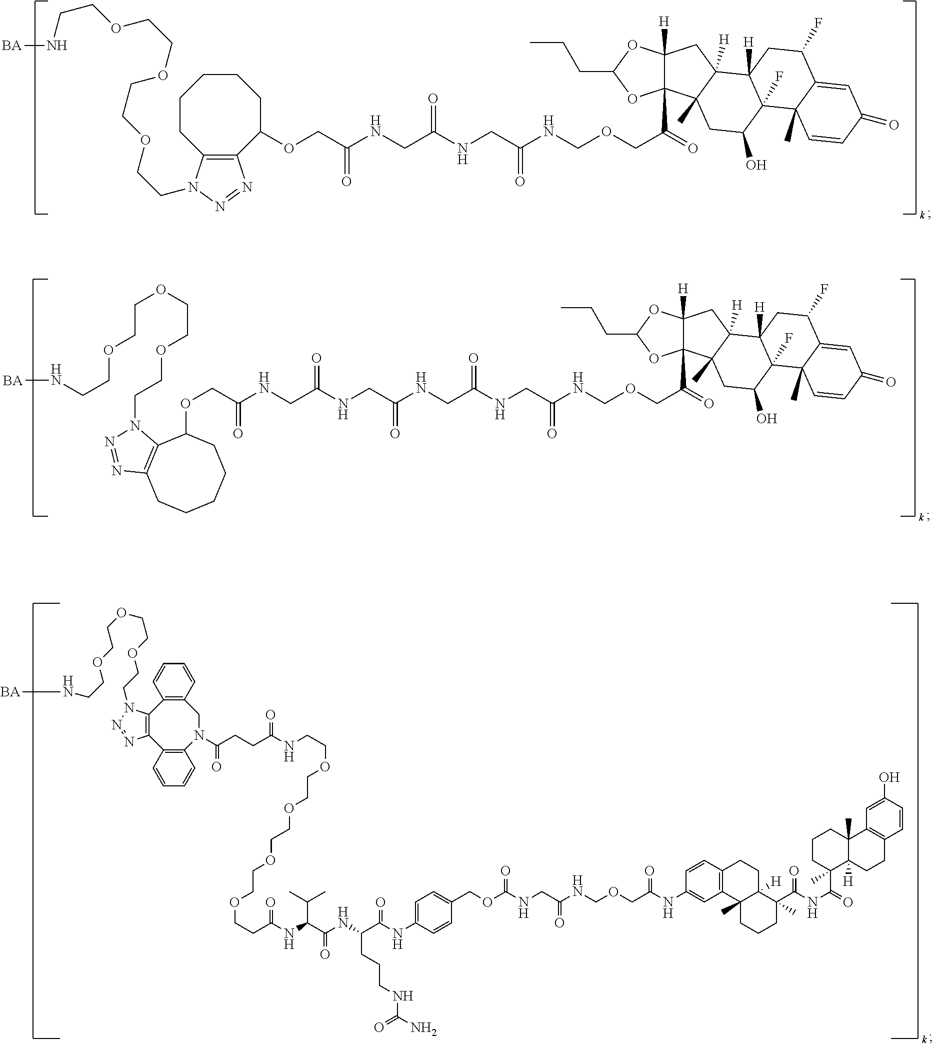 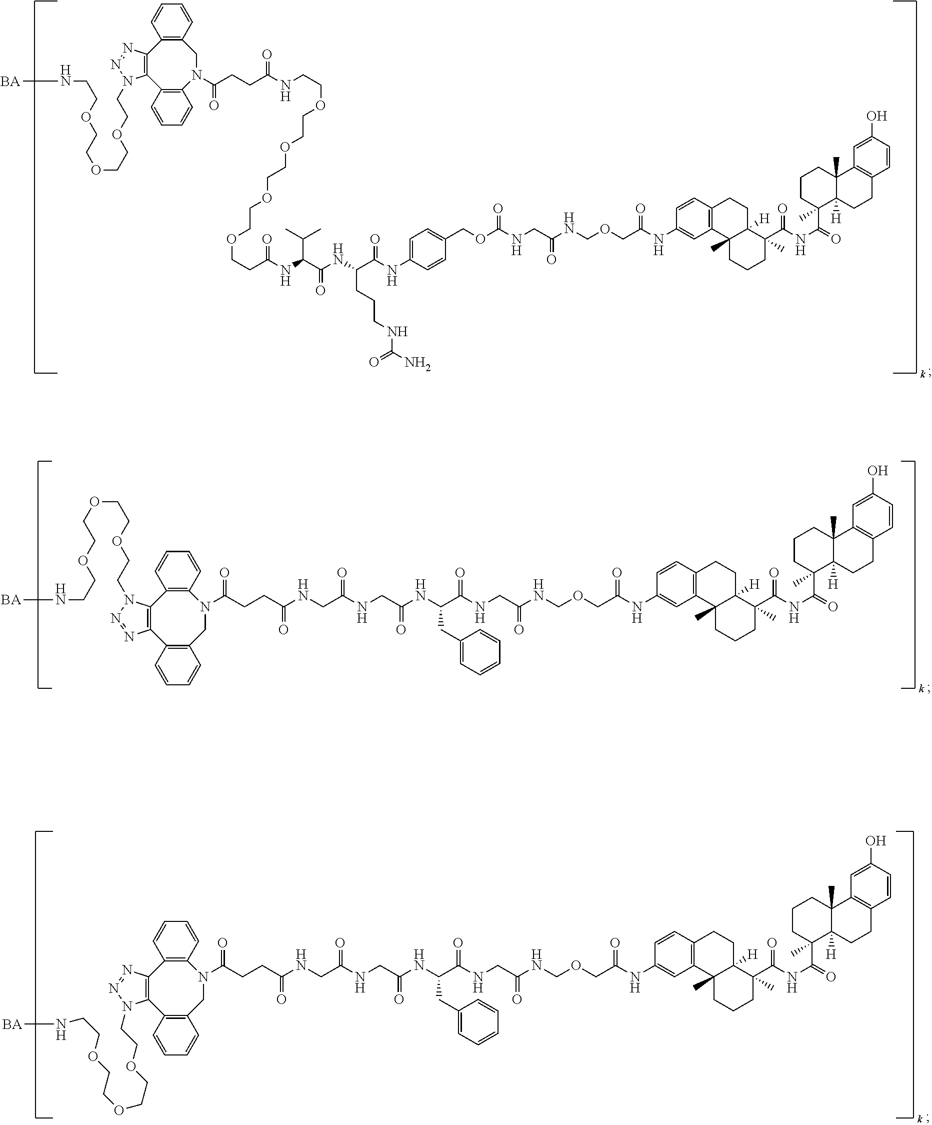
wherein k is 1, 2, 3, or 4.

114. The compound of claim 90, wherein the compound is selected from the group consisting of 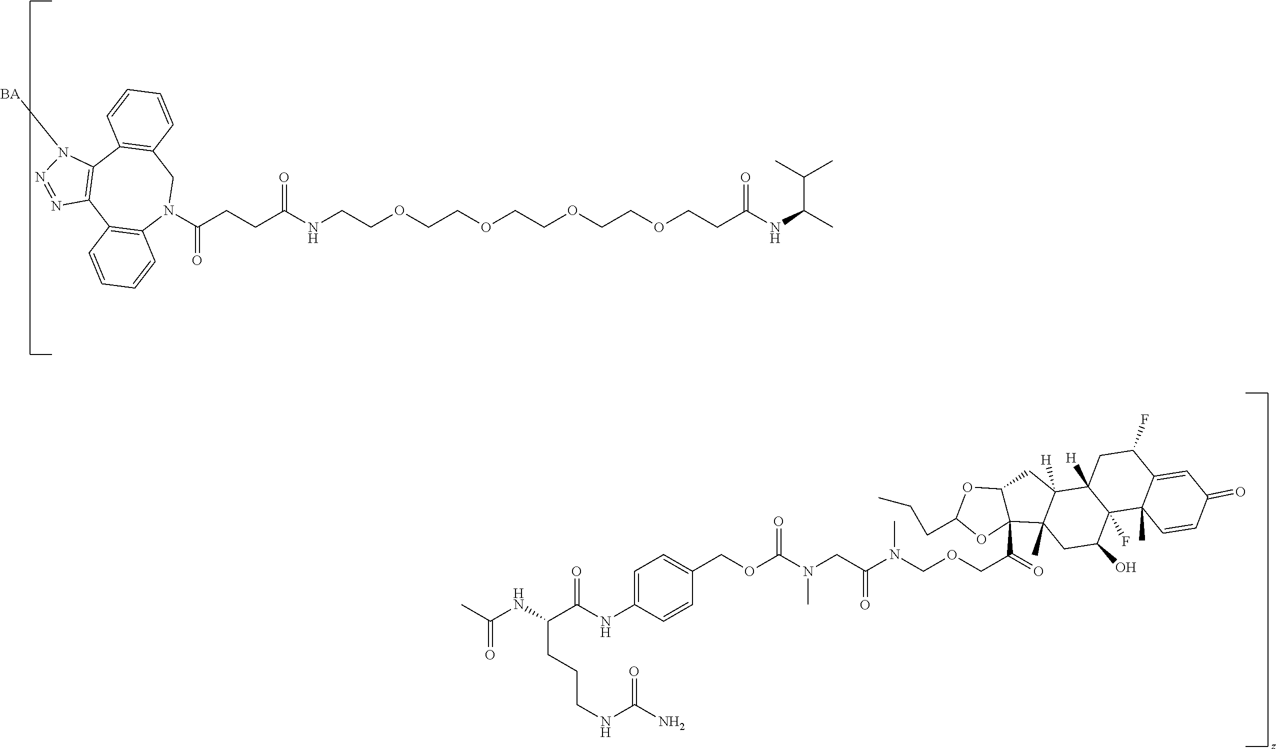 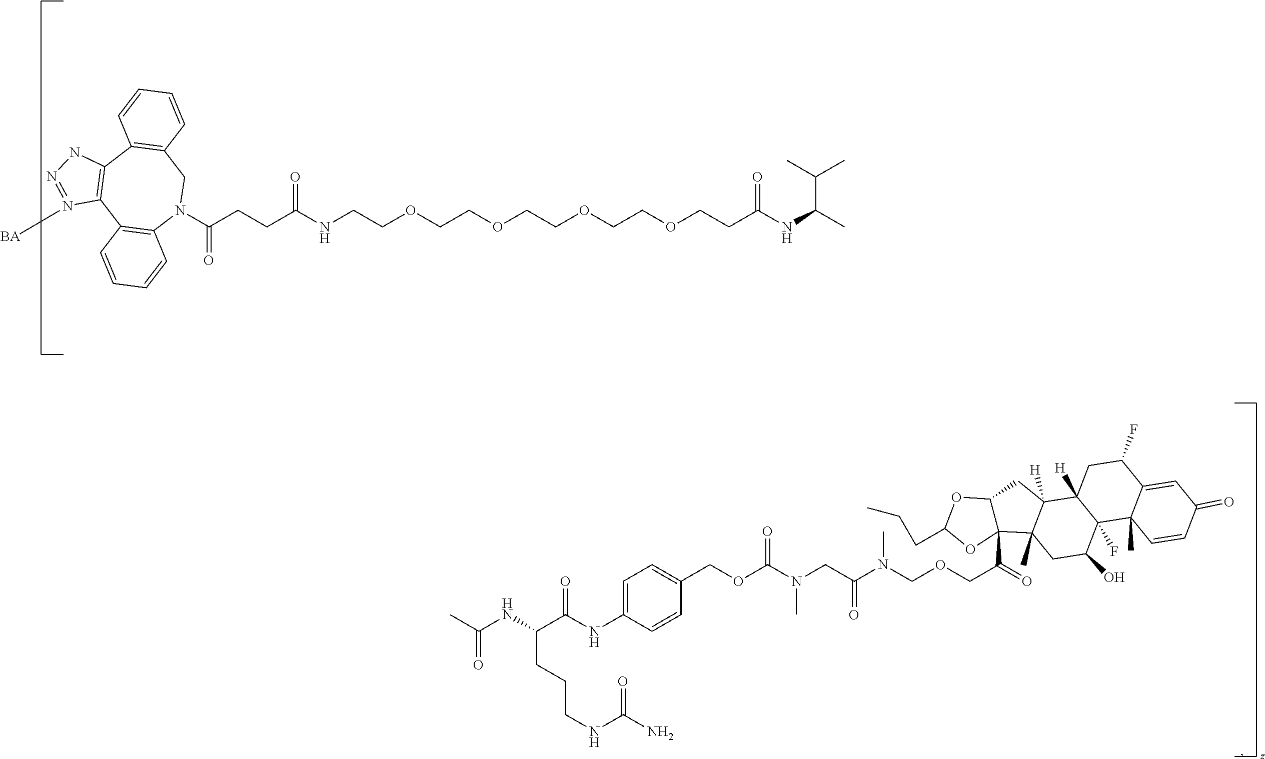 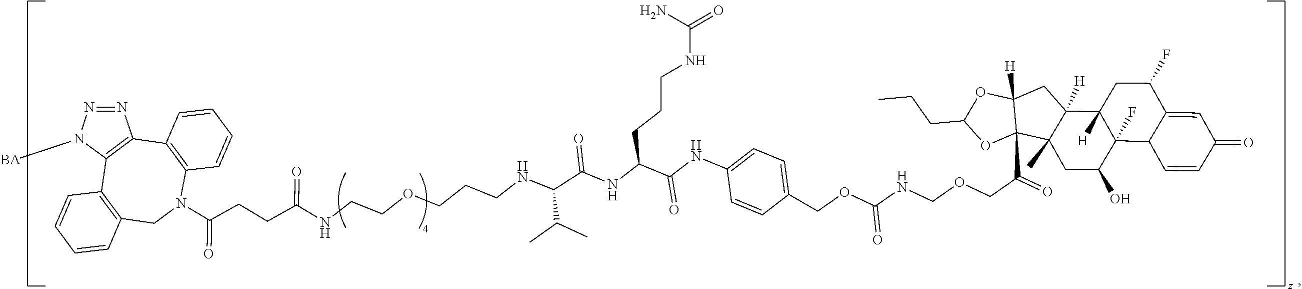 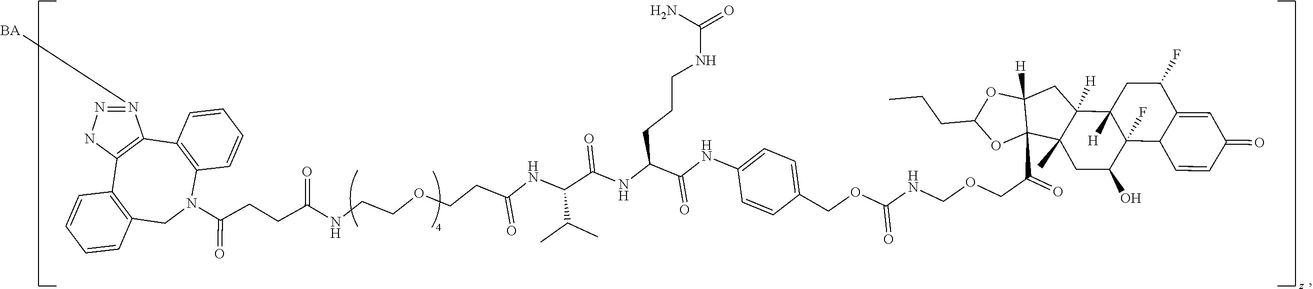 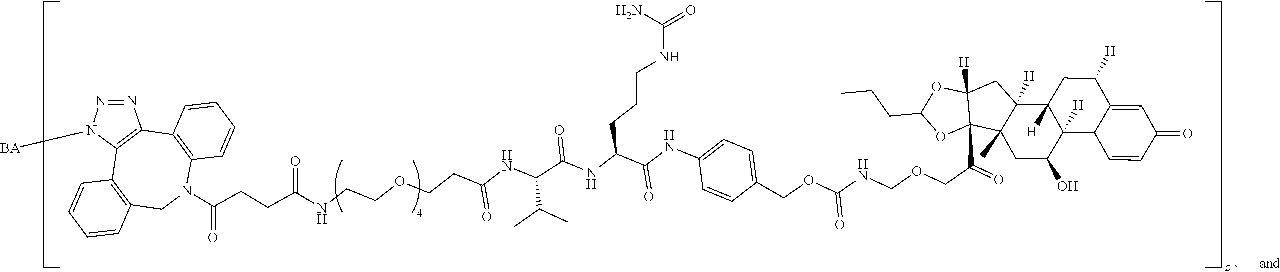 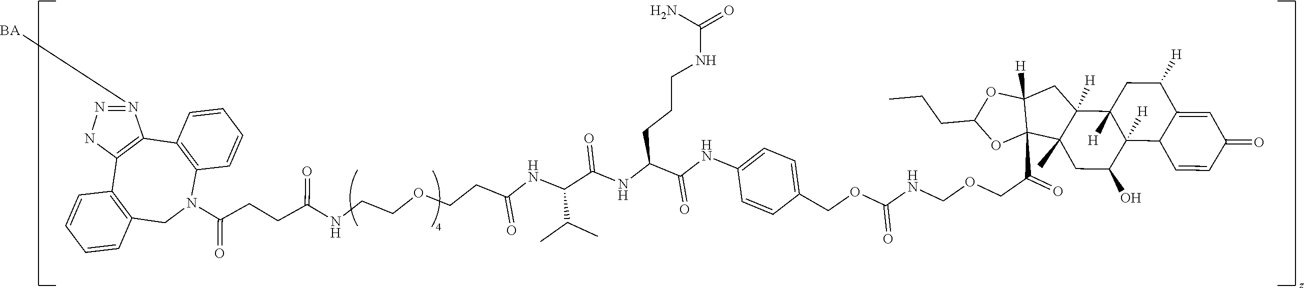 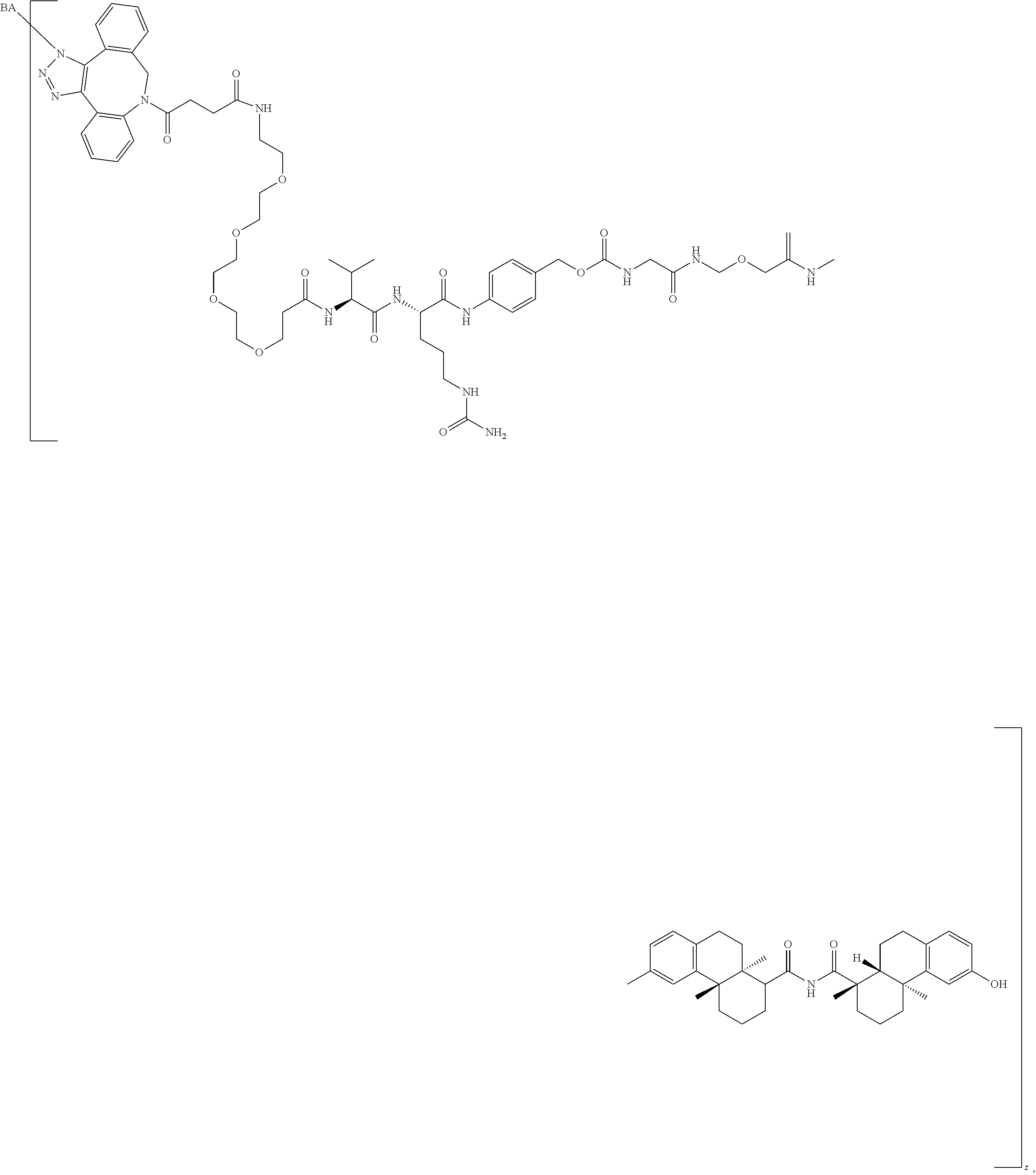 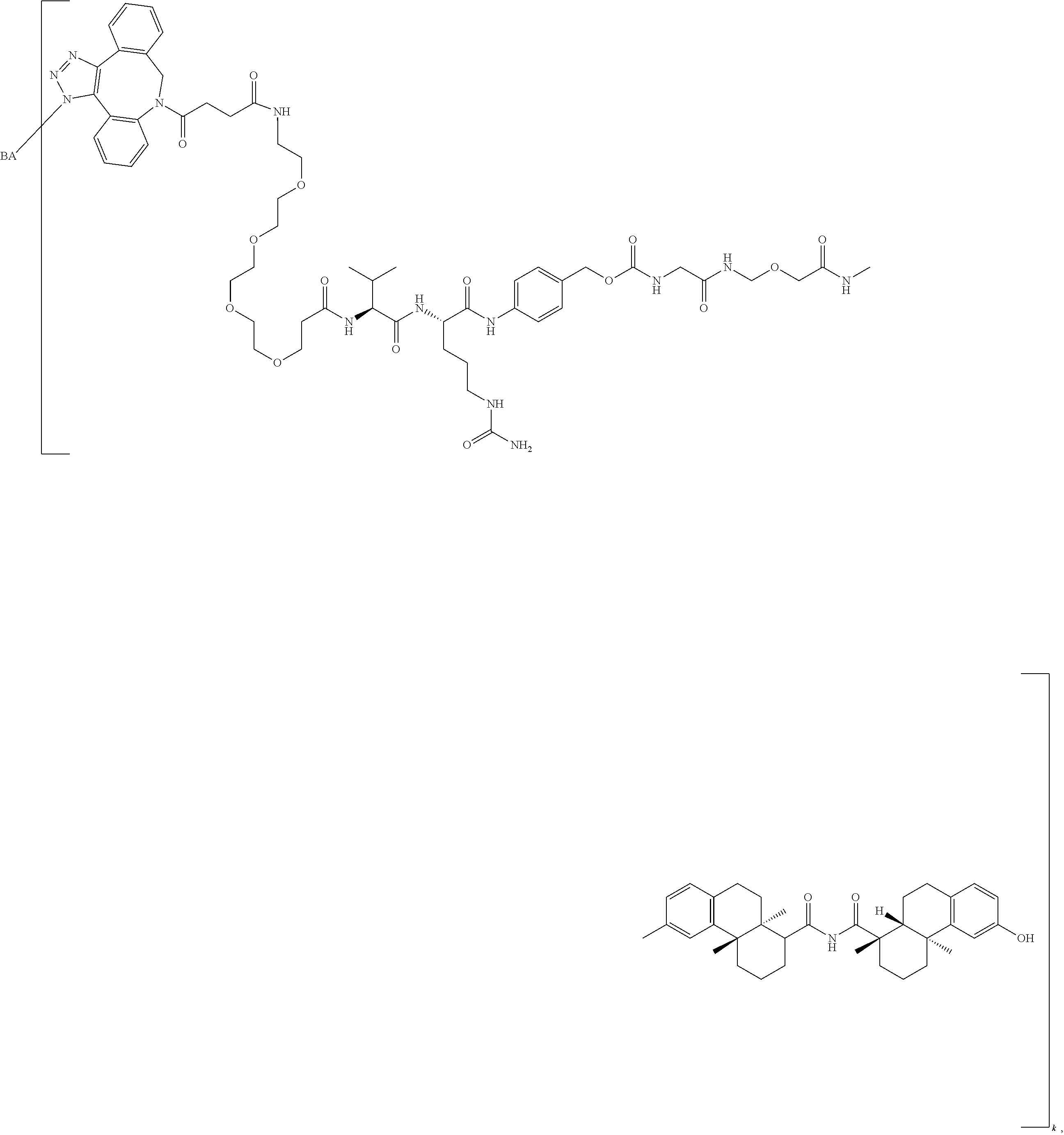 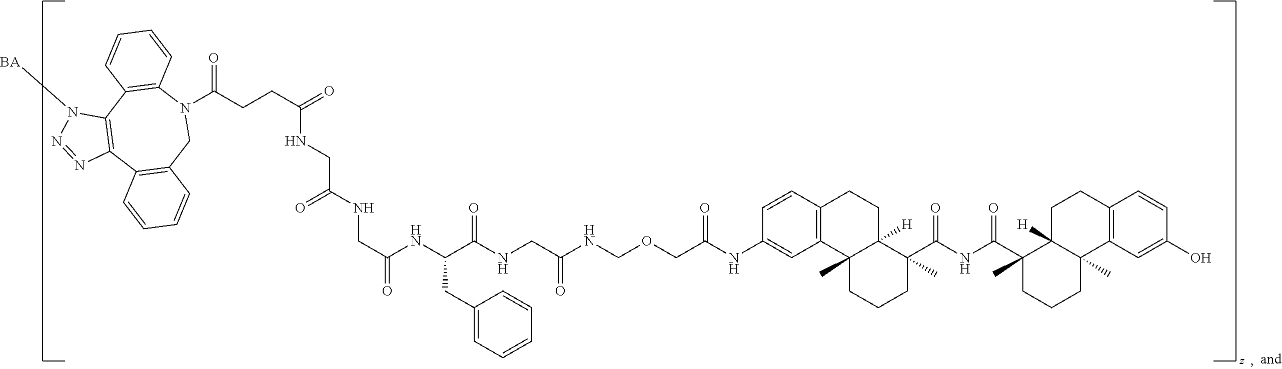 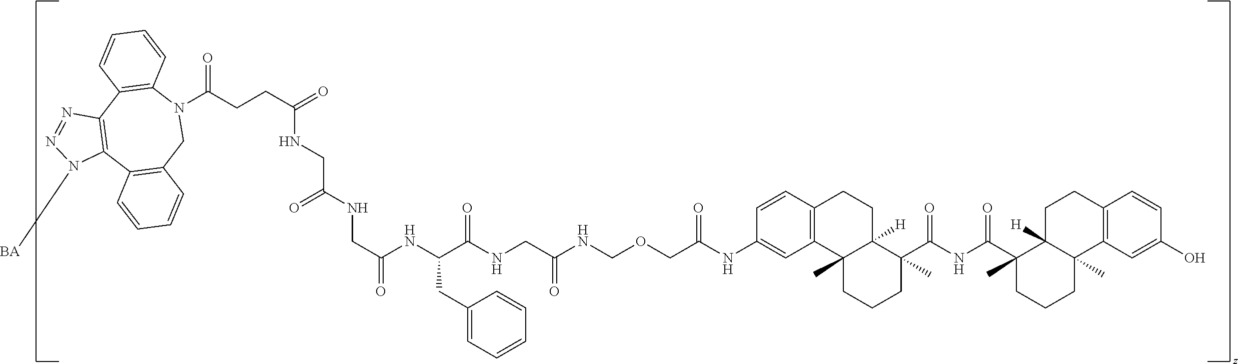
wherein z is 1, 2, 3, or 4.

115. An antibody-drug conjugate comprising an antibody, or an antigen binding fragment thereof, where said antibody or antigen binding fragment thereof is conjugated to a compound of claim 2 or 39.

116. The antibody-drug conjugate of claim 115, wherein the conjugated compound is selected from the group consisting of

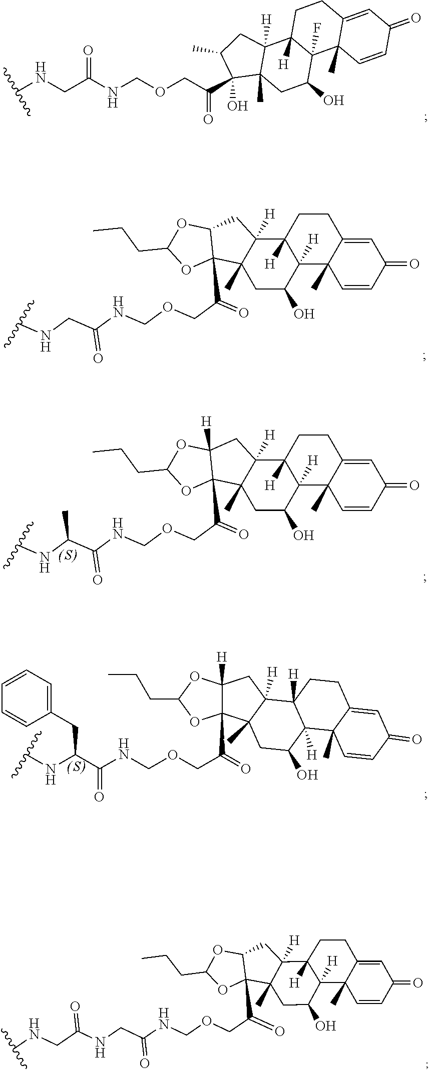

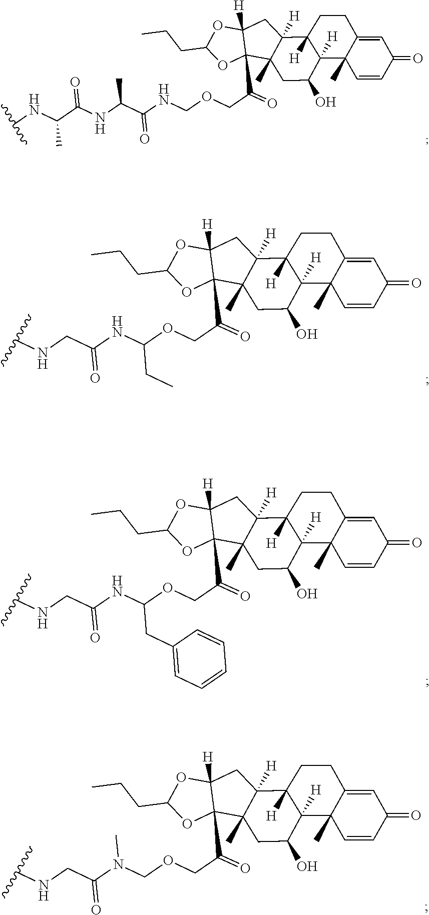

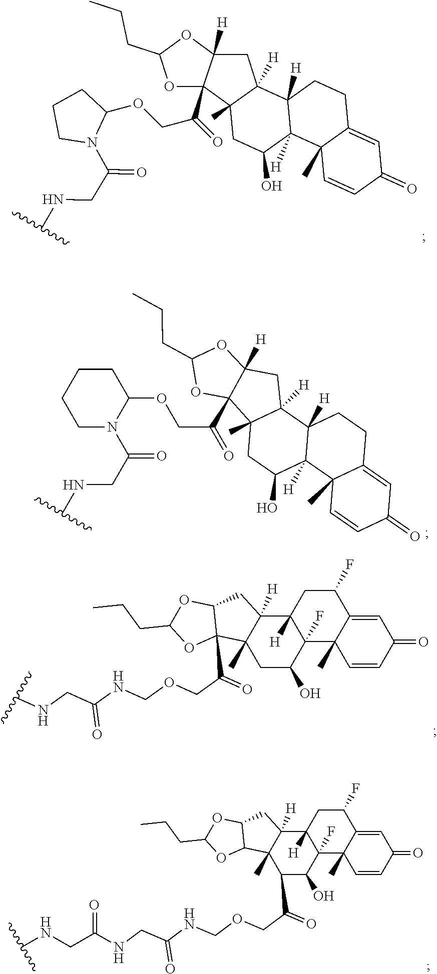

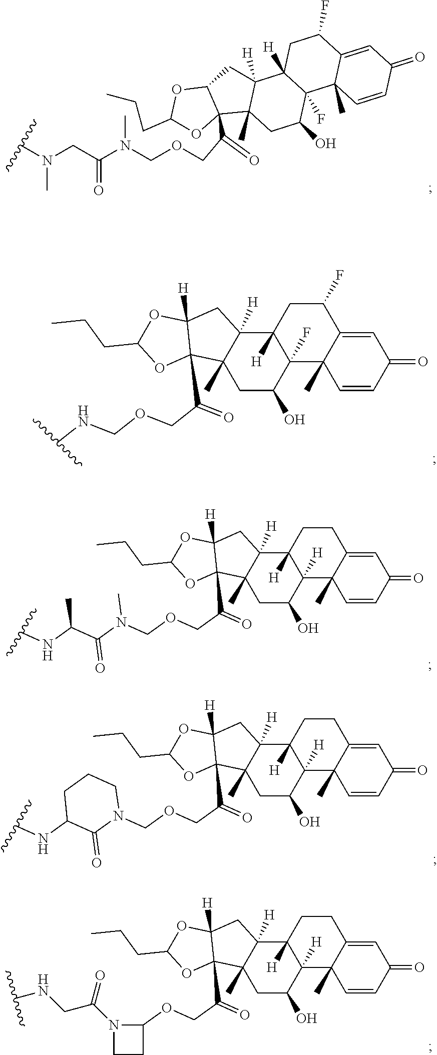

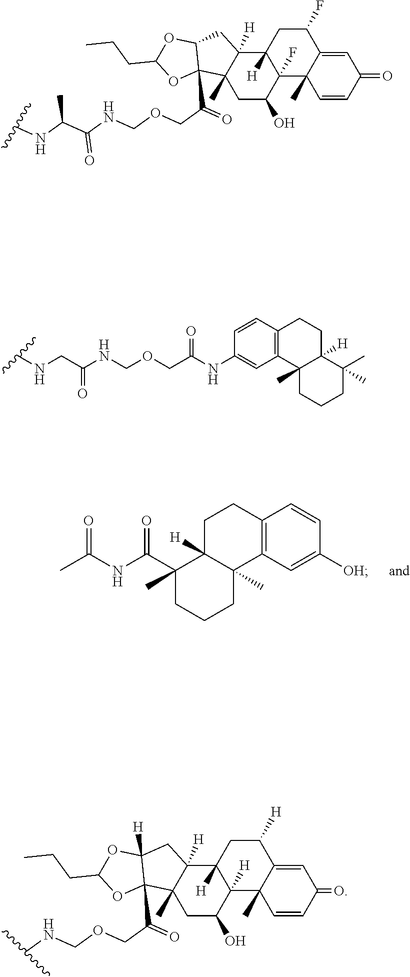

117. The compound of claim 99, wherein BA is an antibody or antigen-binding fragment thereof.

118. The compound of claim 117, wherein BA is a transglutaminase-modified antibody or antigen-binding fragment thereof comprising at least one glutamine residue used for conjugation.

119. The compound of claim 117, wherein BA is a transglutaminase-modified antibody or antigen-binding fragment thereof comprising at least two glutamine residues used for conjugation.

120. The compound of claim 117, wherein BA is a transglutaminase-modified antibody or antigen-binding fragment thereof comprising at least four glutamine residues used for conjugation.

121. The compound of claim 120, wherein BA is a transglutaminase-modified antibody or antigen-binding fragment thereof, wherein conjugation is at two Q295 residues; and k is 2.

122. The compound of claim 120, wherein BA is a transglutaminase-modified antibody or antigen-binding fragment thereof, wherein conjugation is at two Q295 residues and two N297Q residues; and k is 4.

123. A pharmaceutical composition comprising the compound of any preceding claim, and a pharmaceutically acceptable excipient, carrier, or diluent.

124. A method for treating a disease, disorder, or condition associated with glucocorticoid receptor signaling in a subject comprising administering to the subject an effective amount of a compound or pharmaceutical composition of any preceding claim.

125. The method of claim 124, wherein the disease, disorder, or condition, is an inflammatory disease, disorder, or condition.

126. The method of claim 124 or 125, wherein side effects associated with administration of the unconjugated steroid payload of said compound are reduced.

127. A method for treating dyslipidemia, a metabolic disease, inflammation, or a neurodegenerative disease in a subject comprising administering to the subject an effective amount of a compound or pharmaceutical composition of any preceding claim.

128. A method for treating dyslipidemia in a subject comprising administering to the subject an effective amount of a compound or pharmaceutical composition of any preceding claim.

129. A method for treating a metabolic disease in a subject comprising administering to the subject an effective amount of a compound or pharmaceutical composition of any preceding claim.

130. A method for treating inflammation in a subject comprising administering to the subject an effective amount of a compound or pharmaceutical composition of any preceding claim.

131. A method for treating a neurodegenerative disease in a subject comprising administering to the subject an effective amount of a compound or pharmaceutical composition of any preceding claim.

132. A method of preparing an antibody-drug conjugate comprising contacting a binding agent with a compound of claim 39.

133. The compound of claim 2, having the following structure 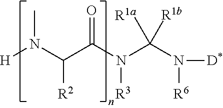
or a pharmaceutically acceptable salt thereof.

134. The compound of claim 2, having the following structure 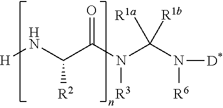
or a pharmaceutically acceptable salt thereof.

135. The compound of claim 2, having the following structure 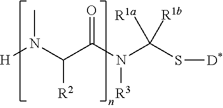
or a pharmaceutically acceptable salt thereof.

136. The compound of claim 2, having the following structure 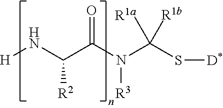
or a pharmaceutically acceptable salt thereof.

137. The compound of claim 39, having the following structure 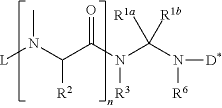
or a pharmaceutically acceptable salt thereof.

138. The compound of claim 39, having the following structure 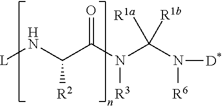
or a pharmaceutically acceptable salt thereof.

139. The compound of claim 39, having the following structure 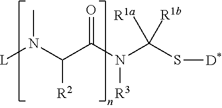
or a pharmaceutically acceptable salt thereof.

140. The compound of claim 39, having the following structure 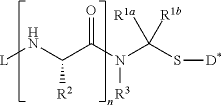
or a pharmaceutically acceptable salt thereof.

141. The compound of claim 99, having the following structure

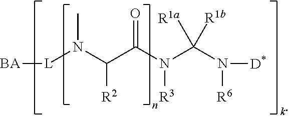

142. The compound of claim 99, having the following structure

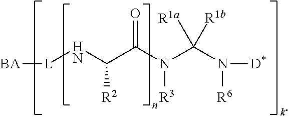

143. The compound of claim 99, having the following structure

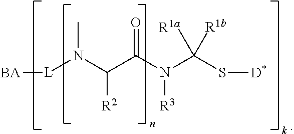

144. The compound of claim 99, having the following structure [chemical structure]


SEQUENCE LISTING (33 sequences)

SEQ ID NO: 1 - protein, 115 aa, Artificial Sequence
  Synthetic H1H7814N, HCVR
QVQLVESGGGVVQPGRSLRLSCVASGFTISSYGMNWVRQAPGKGLEWVAVISYDGGNKYSVDSVKGRFTISRDNSKNTLYLQMNSLRAEDSAVYYCARGRYFDLWGRGTLVTVSS

SEQ ID NO: 2 - protein, 8 aa, Artificial Sequence
  Synthetic H1H7814N, HCDR1
GFTISSYG

SEQ ID NO: 3 - protein, 8 aa, Artificial Sequence
  Synthetic H1H7814N, HCDR2
ISYDGGNK

SEQ ID NO: 4 - protein, 8 aa, Artificial Sequence
  Synthetic H1H7814N, HCDR3
ARGRYFDL

SEQ ID NO: 5 - protein, 107 aa, Artificial Sequence
  Synthetic H1H7814N, LCVR
DIQMTQSPSTLSASVGDRVTITCRASQSISSWLAWYQQKPGRAPNLLISKASSLKSGVPSRFSGSGSGTEFTLTVSSLQPDDFATYYCQQYYSYSYTFGQGTKLEIK

SEQ ID NO: 6 - protein, 6 aa, Artificial Sequence
  Synthetic H1H7814N, LCDR1
QSISSW

SEQ ID NO: 7 - protein, 3 aa, Artificial Sequence
  Synthetic H1H7814N, LCDR2
KAS

SEQ ID NO: 8 - protein, 9 aa, Artificial Sequence
  Synthetic H1H7814N, LCDR3
QQYYSYSYT

SEQ ID NO: 9 - protein, 115 aa, Artificial Sequence
  Synthetic H1H6958N2, HCVR
QVQLVESGGGVVQPGRSLRLSCGASGFTFRNYGMQWVRQGPGKGLEWVTLISFDGNDKYYADSVKGRFTISRDNSKNTLFLQMNSLRTEDTAVYYCARGGDFDYWGQGTLVTVSS

SEQ ID NO: 10 - protein, 8 aa, Artificial Sequence
  Synthetic H1H6958N2, HCDR1
GFTFRNYG

SEQ ID NO: 11 - protein, 8 aa, Artificial Sequence
  Synthetic H1H6958N2, HCDR2
ISFDGNDK

SEQ ID NO: 12 - protein, 8 aa, Artificial Sequence
  Synthetic H1H6958N2, HCDR3
ARGGDFDY

SEQ ID NO: 13 - protein, 108 aa, Artificial Sequence
  Synthetic H1H6958N2, LCVR
DIQMTQSPSSLSASVGDRVTITCRASQDIRKDLGWYQQKPGKAPKRLIYAASSLHSGVPSRFSGSGSGTEFTLTISSLQPEDFATYYCLQHNSYPMYTFGQGTKLEIK

SEQ ID NO: 14 - protein, 6 aa, Artificial Sequence
  Synthetic H1H6958N2, LCDR1
QDIRKD

SEQ ID NO: 15 - protein, 3 aa, Artificial Sequence
  Synthetic H1H6958N2, LCDR2
AAS

SEQ ID NO: 16 - protein, 10 aa, Artificial Sequence
  Synthetic H1H6958N2, LCDR3
LQHNSYPMYT

SEQ ID NO: 17 - protein, 267 aa, Artificial Sequence
  Synthetic hPRLR ecto-MMH,
MHRPRRRGTRPPPLALLAALLLAARGADAQLPPGKPEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEDRKPYLWIKWSPPTLIDLKTGWFTLLYEIRLKPEKAAEWEIHFAGQQTEFKILSLHPGQKYLVQVRCKPDHGYWSAWSPATFIQIPSDFTMNDEQKLISEEDLGGEQKLISEEDLHHHHHH

SEQ ID NO: 18 - DNA, 357 bp, Artificial Sequence
  Synthetic H1H21234N, HCVR
caggtgcagctgcaggagtcgggcccaggactggtgaagccttcggagaccctgtccctcacctgcactgtcactggtggctccatcagtaggaactactggagttggatccggcagcccccagggaagggactggaatggattggatatatctattacagtgggagtatcgactacaatccctccctcaagagtcgagtcaccatatcagtagacacgtccaagaaccagttctccctgaagctgagttctatgaccgctgcggacacggccgtatactactgtgcgagagatcggtggaactggaaatacggtatggacgtctggggccaagggaccacggtcatcgtctcgtca

SEQ ID NO: 19 - protein, 119 aa, Artificial Sequence
  Synthetic H1H21234N, HCVR
QVQLQESGPGLVKPSETLSLTCTVTGGSISRNYWSWIRQPPGKGLEWIGYIYYSGSIDYNPSLKSRVTISVDTSKNQFSLKLSSMTAADTAVYYCARDRWNWKYGMDVWGQGTTVIVSS

SEQ ID NO: 20 - DNA, 24 bp, Artificial Sequence
  Synthetic H1H21234N, HCDR1
ggtggctccatcagtaggaactac

SEQ ID NO: 21 - protein, 8 aa, Artificial Sequence
  Synthetic H1H21234N, HCDR1
GGSISRNY

SEQ ID NO: 22 - DNA, 21 bp, Artificial Sequence
  Synthetic H1H21234N, HCDR2
atctattacagtgggagtatc

SEQ ID NO: 23 - protein, 7 aa, Artificial Sequence
  Synthetic H1H21234N, HCDR2
IYYSGSI

SEQ ID NO: 24 - DNA, 39 bp, Artificial Sequence
  Synthetic H1H21234N, HCDR3
gcgagagatcggtggaactggaaatacggtatggacgtc

SEQ ID NO: 25 - protein, 13 aa, Artificial Sequence
  Synthetic H1H21234N, HCDR3
ARDRWNWKYGMDV

SEQ ID NO: 26 - DNA, 327 bp, Artificial Sequence
  Synthetic H1H21234N, LCVR
gaaattgtgttgacgcagtctccaggcaccctgtctttgtctccaggggaaagagccaccctctcctgcagggccagtcagactgttagaaacaactacttagcctggtaccaccagaaacctggccaggctcccaggctcctcatctatggtgcatccagcagggccactggcatcccagacaggttcagtggcagtgggtctgggacagacttcactctcaccatcagcagactggagcctgaagattttacagtgtattactgtcaccagtatggtaactcaccttggacgttcggccaagggaccaaaatggaaatcaaacga

SEQ ID NO: 27 - protein, 109 aa, Artificial Sequence
  Synthetic H1H21234N, LCVR
EIVLTQSPGTLSLSPGERATLSCRASQTVRNNYLAWYHQKPGQAPRLLIYGASSRATGIPDRFSGSGSGTDFTLTISRLEPEDFTVYYCHQYGNSPWTFGQGTKMEIKR

SEQ ID NO: 28 - DNA, 21 bp, Artificial Sequence
  Synthetic H1H21234N, LCDR1
cagactgttagaaacaactac

SEQ ID NO: 29 - protein, 7 aa, Artificial Sequence
  Synthetic H1H21234N, LCDR1
QTVRNNY

SEQ ID NO: 30 - DNA, 9 bp, Artificial Sequence
  Synthetic H1H21234N, LCDR2
ggtgcatcc

SEQ ID NO: 31 - protein, 3 aa, Artificial Sequence
  Synthetic H1H21234N, LCDR2
GAS

SEQ ID NO: 32 - DNA, 27 bp, Artificial Sequence
  Synthetic H1H21234N, LCDR3
caccagtatggtaactcaccttggacg

SEQ ID NO: 33 - protein, 9 aa, Artificial Sequence
  Synthetic H1H21234N, LCDR3
HQYGNSPWT